SEQUENCE LISTING (277 sequences)

SEQ ID NO: 1 - DNA, 6159 bp, Homo sapiens
atgttgaagttcaaatatggagcgcggaatcctttggatgctggtgctgctgaacccattgccagccgggcctccaggctgaatctgttcttccaggggaaaccaccctttatgactcaacagcagatgtctcctctttcccgagaagggatattagatgccctctttgttctctttgaagaatgcagtcagcctgctctgatgaagattaagcacgtgagcaactttgtcccggaagtgtattccgacaccatagctgagttacaggagctccagccttcggcaaaggacttcgaagtcagaagtcttgtaggttgtggtcactttgctgaagtgcaggtggtaagagagaaagcaaccggggacatctatgctatgaaagtgatgaagaagaaggctttattggcccaggagcaggtttcattttttgaggaagagcggaacatattatctcgaagcacaagcccgtggatcccccaattacagtatgcctttcaggacaaaaatcacctttatctggtcatggaatatcagcctggaggggacttgctgtcacttttgaatagatatgaggaccagttagatgaaaacctgatacagttttacctagctgagctgattttggctgttcacagcgttcatctgatgggatacgtgcatcgagacatcaagcctgagaacattctcgttgaccgcacaggacacatcaagctggtggattttggatctgccgcgaaaatgaattcaaacaagatggtgaatgccaaactcccgattgggaccccagattacatggctcctgaagtgctgactgtgatgaacggggatggaaaaggcacctacggcctggactgtgactggtggtcagtgggcgtgattgcctatgagatgatttatgggagatcccccttcgcagagggaacctctgccagaaccttcaataacattatgaatttccagcggtttttgaaatttccagatgaccccaaagtgagcagtgactttcttgatctgattcaaagcttgttgtgcggccagaaagagagactgaagtttgaaggtctttgctgccatcctttcttctctaaaattgactggaacaacattcgtaactctcctccccccttcgttcccaccctcaagtctgacgatgacacctccaattttgatgaaccagagaagaattcgtgggtttcatcctctccgtgccagctgagcccctcaggcttctcgggtgaagaactgccgtttgtggggttttcgtacagcaaggcactggggattcttggtagatctgagtctgttgtgtcgggtctggactcccctgccaagactagctccatggaaaagaaacttctcatcaaaagcaaagagctacaagactctcaggacaagtgtcacaagatggagcaggaaatgacccggttacatcggagagtgtcagaggtggaggctgtgcttagtcagaaggaggtggagctgaaggcctctgagactcagagatccctcctggagcaggaccttgctacctacatcacagaatgcagtagcttaaagcgaagtttggagcaagcacggatggaggtgtcccaggaggatgacaaagcactgcagcttctccatgatatcagagagcagagccggaagctccaagaaatcaaagagcaggagtaccaggctcaagtggaagaaatgaggttgatgatgaatcagttggaagaggatcttgtctcagcaagaagacggagtgatctctacgaatctgagctgagagagtctcggcttgctgctgaagaattcaagcggaaagcgacagaatgtcagcataaactgttgaaggctaaggatcaagggaagcctgaagtgggagaatatgcgaaactggagaagatcaatgctgagcagcagctcaaaattcaggagctccaagagaaactggagaaggctgtaaaagccagcacggaggccaccgagctgctgcagaatatccgccaggcaaaggagcgagccgagagggagctggagaagctgcagaaccgagaggattcttctgaaggcatcagaaagaagctggtggaagctgaggaacgccgccattctctggagaacaaggtaaagagactagagaccatggagcgtagagaaaacagactgaaggatgacatccagacaaaatcccaacagatccagcagatggctgataaaattctggagctcgaagagaaacatcgggaggcccaagtctcagcccagcacctagaagtgcacctgaaacagaaagagcagcactatgaggaaaagattaaagtgttggacaatcagataaagaaagacctggctgacaaggagacactggagaacatgatgcagagacacgaggaggaggcccatgagaagggcaaaattctcagcgaacagaaggcgatgatcaatgctatggattccaagatcagatccctggaacagaggattgtggaactgtctgaagccaataaacttgcagcaaatagcagtctttttacccaaaggaacatgaaggcccaagaagagatgatttctgaactcaggcaacagaaattttacctggagacacaggctgggaagttggaggcccagaaccgaaaactggaggagcagctggagaagatcagccaccaagaccacagtgacaagaatcggctgctggaactggagacaagattgcgggaggtcagtctagagcacgaggagcagaaactggagctcaagcgccagctcacagagctacagctctccctgcaggagcgcgagtcacagttgacagccctgcaggctgcacgggcggccctggagagccagcttcgccaggcgaagacagagctggaagagaccacagcagaagctgaagaggagatccaggcactcacggcacatagagatgaaatccagcgcaaatttgatgctcttcgtaacagctgtactgtaatcacagacctggaggagcagctaaaccagctgaccgaggacaacgctgaactcaacaaccaaaacttctacttgtccaaacaactcgatgaggcttctggcgccaacgacgagattgtacaactgcgaagtgaagtggaccatctccgccgggagatcacggaacgagagatgcagcttaccagccagaagcaaacgatggaggctctgaagaccacgtgcaccatgctggaggaacaggtcatggatttggaggccctaaacgatgagctgctagaaaaagagcggcagtgggaggcctggaggagcgtcctgggtgatgagaaatcccagtttgagtgtcgggttcgagagctgcagagaatgctggacaccgagaaacagagcagggcgagagccgatcagcggatcaccgagtctcgccaggtggtggagctggcagtgaaggagcacaaggctgagattctcgctctgcagcaggctctcaaagagcagaagctgaaggccgagagcctctctgacaagctcaatgacctggagaagaagcatgctatgcttgaaatgaatgcccgaagcttacagcagaagctggagactgaacgagagctcaaacagaggcttctggaagagcaagccaaattacagcagcagatggacctgcagaaaaatcacattttccgtctgactcaaggactgcaagaagctctagatcgggctgatctactgaagacagaaagaagtgacttggagtatcagctggaaaacattcaggttctctattctcatgaaaaggtgaaaatggaaggcactatttctcaacaaaccaaactcattgattttctgcaagccaaaatggaccaacctgctaaaaagaaaaaggttcctctgcagtacaatgagctgaagctggccctggagaaggagaaagctcgctgtgcagagctagaggaagcccttcagaagacccgcatcgagctccggtccgcccgggaggaagctgcccaccgcaaagcaacggaccacccacacccatccacgccagccaccgcgaggcagcagatcgccatgtccgccatcgtgcggtcgccagagcaccagcccagtgccatgagcctgctggccccgccatccagccgcagaaaggagtcttcaactccagaggaatttagtcggcgtcttaaggaacgcatgcaccacaatattcctcaccgattcaacgtaggactgaacatgcgagccacaaagtgtgctgtgtgtctggataccgtgcactttggacgccaggcatccaaatgtctcgaatgtcaggtgatgtgtcaccccaagtgctccacgtgcttgccagccacctgcggcttgcctgctgaatatgccacacacttcaccgaggccttctgccgtgacaaaatgaactccccaggtctccagaccaaggagcccagcagcagcttgcacctggaagggtggatgaaggtgcccaggaataacaaacgaggacagcaaggctgggacaggaagtacattgtcctggagggatcaaaagtcctcatttatgacaatgaagccagagaagctggacagaggccggtggaagaatttgagctgtgccttcccgacggggatgtatctattcatggtgccgttggtgcttccgaactcgcaaatacagccaaagcagatgtcccatacatactgaagatggaatctcacccgcacaccacctgctggcccgggagaaccctctacttgctagctcccagcttccctgacaaacagcgctgggtcaccgccttagaatcagttgtcgcaggtgggagagtttctagggaaaaagcagaagctgatgctaaactgcttggaaactccctgctgaaactggaaggtgatgaccgtctagacatgaactgcacgctgcccttcagtgaccaggtggtgttggtgggcaccgaggaagggctctacgccctgaatgtcttgaaaaactccctaacccatgtcccaggaattggagcagtcttccaaatttatattatcaaggacctggagaagctactcatgatagcaggagaagagcgggcactgtgtcttgtggacgtgaagaaagtgaaacagtccctggcccagtcccacctgcctgcccagcccgacatctcacccaacatttttgaagctgtcaagggctgccacttgtttggggcaggcaagattgagaacgggctctgcatctgtgcagccatgcccagcaaagtcgtcattctccgctacaacgaaaacctcagcaaatactgcatccggaaagagatagagacctcagagccctgcagctgtatccacttcaccaattacagtatcctcattggaaccaataaattctacgaaatcgacatgaagcagtacacgctcgaggaattcctggataagaatgaccattccttggcacctgctgtgtttgccgcctcttccaacagcttccctgtctcaatcgtgcaggtgaacagcgcagggcagcgagaggagtacttgctgtgtttccacgaatttggagtgttcgtggattcttacggaagacgtagccgcacagacgatctcaagtggagtcgcttacctttggcctttgcctacagagaaccctatctgtttgtgacccacttcaactcactcgaagtaattgagatccaggcacgctcctcagcagggacccctgcccgagcgtacctggacatcccgaacccgcgctacctgggccctgccatttcctcaggagcgatttacttggcgtcctcataccaggataaattaagggtcatttgctgcaagggaaacctcgtgaaggagtccggcactgaacaccaccggggcccgtccacctcccgcagcagccccaacaagcgaggcccacccacgtacaacgagcacatcaccaagcgcgtggcctccagcccagcgccgcccgaaggccccagccacccgcgagagccaagcacaccccaccgctaccgcgaggggcggaccgagctgcgcagggacaagtctcctggccgccccctggagcgagagaagtcccccggccggatgctcagcacgcggagagagcggtcccccgggaggctgtttgaagacagcagcaggggccggctgcctgcgggagccgtgaggaccccgctgtcccaggtgaacaaggtgaggcagcattcc

SEQ ID NO: 2 - DNA, 1662 bp, Homo sapiens
atggtggacatgggggccctggacaacctgatcgccaacaccgcctacctgcaggcccggaagccctcggactgcgacagcaaagagctgcagcggcggcggcgtagcctggccctgcccgggctgcagggctgcgcggagctccgccagaagctgtccctgaacttccacagcctgtgtgagcagcagcccatcggtcgccgcctcttccgtgacttcctagccacagtgcccacgttccgcaaggcggcaaccttcctagaggacgtgcagaactgggagctggccgaggagggacccaccaaagacagcgcgctgcaggggctggtggccacttgtgcgagtgcccctgccccggggaacccgcaacccttcctcagccaggccgtggccaccaagtgccaagcagccaccactgaggaagagcgagtggctgcagtgacgctggccaaggctgaggccatggctttcttgcaagagcagccctttaaggatttcgtgaccagcgccttctacgacaagtttctgcagtggaaactcttcgagatgcaaccagtgtcagacaagtacttcactgagttcagagtgctggggaaaggtggttttggggaggtatgtgccgtccaggtgaaaaacactgggaagatgtatgcctgtaagaaactggacaagaagcggctgaagaagaaaggtggcgagaagatggctctcttggaaaaggaaatcttggagaaggtcagcagccctttcattgtctctctggcctatgcctttgagagcaagacccatctctgccttgtcatgagcctgatgaatgggggagacctcaagttccacatctacaacgtgggcacgcgtggcctggacatgagccgggtgatcttttactcggcccagatagcctgtgggatgctgcacctccatgaactcggcatcgtctatcgggacatgaagcctgagaatgggcttctggatgacctcggcaactgcaggttatctgacctggggctggccgtggagatgaagggtggcaagcccatcacccagagggctggaaccaatggttacatggctcctgagatcctaatggaaaaggtaagttattcctatcctgtggactggtttgccatgggatgcagcatttatgaaatggttgctggacgaacaccattcaaagattacaaggaaaaggtcagtaaagaggatctgaagcaaagaactctgcaagacgaggtcaaattccagcatgataacttcacagaggaagcaaaagatatttgcaggctcttcttggctaagaaaccagagcaacgcttaagaagcagagaaaagtctgatgatcccaggaaacatcatttctttaaaacgatcaactttcctcgcctggaagctggcctaattgaacccccatttgtgccagacccttcagtggtttatgccaaagacatcgctgaaattgatgatttctctgaggttcggggggtggaatttgatgacaaagataagcagttcttcaaaaactttgcgacaggtgctgttcctatagcatggcaggaagaaattatagaaacgggactgtttgaggaactgaatgaccccaacagacctacgggttgtgaggagggtaattcatccaagtctggcgtgtgtttgttattgtaa

SEQ ID NO: 3 - DNA, 7785 bp, Homo sapiens
atgcagacaaaacacttcctggtggtgactcatgttactccaaagcatctccacccaagcacctgttcagctgtgacatcttacacgcagtgttctgtccatgcgtgctcacctcctgccccctcaggggcagccagtcctccctcaagttgccgaacaagcaaccggaaaagcttaataggcaatgggcagtcaccagcattgcctcgaccacactcacctctctctgctcatgcagagtctctttttgagccatacatgagaattgtttgcaacttgctcaccttgcgcttcagctatatcagttccactcccctcaatcttgcaactcgttcattagatgagatactaaattggacggtggaaatagccctcaagatagtccaagaaatttctcccccagtgcctcagcccatttttcatttgcacggagatgcaatccttcaccttcctgggctccttcatccctctcacatgcatagcttgtcctcctctgcccaggaaaaacaccgagaaaacaaaatggcaccttcagccaagtttctgaatattcaggataaactcggtttatggtctgaatgtcctagatatttgttcagttttcctcagagacctaactatatcctaaagtggattaaagagaagttgcatcagttaccataccaaccaacaccagacgagttacacttcttatcaaaacatttctgtaccaccgaaagcatcgccactgagaacagatgcaggaacacgccgatgcgcccccgttcccgaagtctgagacattgtatttctaaacctattaatttagaagagaaggcacagttgtcgtgttctccagttagactggctacagctcagatggaagaacgtctaaaggaaattatcaccagctactctcctgacaacgttctacccttagcagatggagtgcttagtttcactcaccaccagattattgaactggctcgagattgcttggataaatcccaccagggcctcatcacctcacgatacttccttgaattacagcacaaattagataagttgctacaggaggctcatgatcgttcagaaagtggagaattggcatttattaaacaactagttcgaaagatcctaattgttattgcccgccctgctcggttattagagtgcctggaatttgatccggaagaattttactacctattggaagcagcagaaggccatgccaaagaaggacagggtattaaaaccgacattcccaggtacatcattagccaactgggactcaataaggatcccttggaagaaatggctcatttgggaaactacgatagtgggacagcagaaacaccagaaacagatgaatcagtgagtagctctaatgcctccctgaaacttcgaaggaaacctcgggaaagtgattttgaaacgattaaattgattagcaatggagcctatggggcagtctactttgttcggcataaagaatcccggcagaggtttgccatgaagaagattaataaacagaacctcatccttcgaaaccagatccagcaggcctttgtggagcgggatatcctgacttttgcagaaaacccctttgttgtcagcatgtattgctcctttgaaacaaggcgccacttgtgcatggtcatggaatatgtggaagggggagactgtgctactttaatgaaaaacatgggtcctctccctgttgatatggccagaatgtactttgctgagacggtcttggccttggaatatttacataattatggaattgtacacagggatttgaaaccagacaacttgttggttacctccatggggcacataaagctgacagattttggattatctaaggtgggactaatgagcatgactaccaacctttacgagggtcatattgagaaggatgctagagagttcctggataaacaggtctgtggcacacctgaatacattgcaccagaagtgattctgaggcagggttatggaaagccggtggactggtgggccatggggattatcctctatgaatttctggttggatgcgtgccattctttggggatactccagaggagctatttggacaagtcatcagtgatgagatcaactggcctgagaaggatgaggcacccccacctgatgcccaggatctgattaccttactcctcaggcagaatcccctggagaggctgggaacaggtggtgcatatgaagtcaaacagcatcgattcttccgttctttagactggaacagtttgctgagacagaaggcagaatttattccccaactggaatctgaggatgacacaagttattttgatactcggtctgagaagtatcatcatatggaaacggaggaagaagatgacacaaatgatgaagactttaatgtggaaataaggcagttttcttcatgttcacacaggttttcaaaagttttcagcagtatagatcgaatcactcagaattcagcagaagagaaggaagactctgtggacaaaaccaaaagcaccaccttgccatccacagaaacactgagctggagttcagaatattctgaaatgcaacagctatcaacatccaactcttcagatactgaaagcaacagacataaactcagttctggcctacttcccaaactggctatttcaacagagggagagcaagatgaagctgcctcctgccctggagacccccatgaggagccaggaaagccagcccttcctcctgaagagtgtgcccaggaggagcctgaggtcaccaccccagccagcaccatcagcagctccaccctgtcagttggcagtttttcagagcacttggatcagataaatggacgaagcgagtgtgtggacagtacagataattcctcaaagccatccagtgaacccgcttctcacatggctcggcagcgattagaaagcacagaaaaaaagaaaatctcggggaaagtcacaaagtccctctctgccagtgctctttccctcatgatcccaggagatatgtttgctgtttcccctctgggaagtccaatgtctccccattccctgtcctcggacccttcttcttcacgagattcctctcccagccgagattcctcagcagcttctgccagtccacatcagccgattgtgatccacagttcggggaagaactacggctttaccatccgagccatccgggtgtatgtgggagacagtgacatctatacagtgcaccatatcgtctggaatgtagaagaaggaagtccggcatgccaggcaggactgaaggctggagatcttatcactcacatcaatggagaaccagtgcatggacttgtccacacagaagttatagaactcctactgaagagtgggaataaggtgtcaatcactactaccccatttgaaaacacatcaatcaaaactggaccagccaggagaaacagctataagagccggatggtgaggcggagcaagaaatccaagaagaaagaaagtctcgaaaggaggagatctcttttcaaaaagctagccaagcagccttctcctttactccacaccagccgaagtttctcctgcttgaacagatccctgtcatcgggtgagagcctcccaggttcccccactcatagcttgtctccccggtctccaacaccaagctaccgctccacccctgacttcccatctggtactaattcctcccagagcagctcccctagttctagtgcccccaattccccagcagggtccgggcacatccggcccagcactctccacggtcttgcacccaaactcggcgggcagcggtaccggtccggaaggcgaaagtccgccggcaacatcccactgtccccgctggcccggacgccctctccaaccccgcaacccacctccccgcagcggtcaccatcccctcttctgggacactcactgggcaattccaagatcgcgcaagcctttcccagcaagatgcactccccgcccaccatcgtcagacacatcgtgaggcccaagagtgcggagccccccaggtccccgctgctcaagcgcgtgcagtccgaggagaagctgtcgccctcttacggcagtgacaagaagcacctgtgctcccgcaagcacagcctggaggtgacccaagaggaggtgcagcgggagcagtcccagcgggaggcgccgctgcagagcctggatgagaacgtgtgcgacgtgccgccgctcagccgcgcccggccagtggagcaaggctgcctgaaacgcccagtctcccggaaggtgggccgccaggagtctgtggacgacctggaccgcgacaagctgaaggccaaggtggtggtgaagaaagcagacggcttcccagagaaacaggaatcccaccagaaatcccatggacccgggagtgatttggaaaactttgctctgtttaagctggaagagagagagaagaaagtctatccgaaggctgtggaaaggtcaagtacttttgaaaacaaagcgtctatgcaggaggcgccaccgctgggcagcctgctgaaggatgctcttcacaagcaggccagcgtgcgcgccagcgagggtgcgatgtcggatggcccggtgcctgcggagcaccgccagggtggcggggacttcagacgggcccccgctcctggcaccctccaggatggtctctgccactccctcgacaggggcatctctgggaagggggaaggcacggagaagtcctcccaggccaaggagcttctccgatgtgaaaagttagacagcaagctggccaacatcgattacctccgaaagaaaatgtcacttgaggacaaagaggacaacctctgccctgtgctgaagcccaagatgacagctggctcccacgaatgcctgccagggaacccagtccgacccacgggtgggcagcaggagcccccgccggcttctgagagccgagcttttgtcagcagcacccatgcagctcagatgagtgccgtctcttttgttcccctcaaggccttaacaggccgggtggacagtggaacggagaagcctggcttggttgctcctgagtcccctgttaggaagagcccctccgagtataagctggaaggtaggtctgtctcatgcctggagccgatcgagggcactctggacattgctctcctgtccggacctcaggcctccaagacagaactgccttccccagagtctgcacagagccccagcccaagtggtgacgtgagggcctctgtgccaccagttctccccagcagcagtgggaaaaagaacgataccaccagtgcaagagagctttctccttccagcttaaagatgaataaatcctacctgctggagccttggttcctgccccccagccgaggtctccagaattcaccagcagtttccctgcctgacccagagttcaagagggacaggaaaggtccccatcctactgccaggagccctggaacagtcatggaaagcaatccccaacagagagagggcagctcccctaaacaccaagaccacaccactgaccccaagcttctgacctgcctggggcagaacctccacagccctgacctggccaggccacgctgcccgctcccacctgaagcttccccctcaagggagaagccaggcctgagggaatcgtctgaaagaggccctcccacagccagaagcgagcgctctgctgcgagggctgacacatgcagagagccctccatggaactgtgctttccagaaactgcgaaaaccagtgacaactccaaaaatctcctctctgtgggaaggacccacccagatttctatacacagacccaggccatggagaaagcatgggcgccgggtgggaaaacgaaccacaaagatggcccaggtgaggcgaggcccccgcccagagacaactcctctctgcactcagctggaattccctgtgagaaggagctgggcaaggtgaggcgtggcgtggaacccaagcccgaagcgcttcttgccaggcggtctctgcagccacctggaattgagagtgagaagagtgaaaagctctccagtttcccatctttgcagaaagatggtgccaaggaacctgaaaggaaggagcagcctctacaaaggcatcccagcagcatccctccgccccctctgacggccaaagacctgtccagcccggctgccaggcagcattgcagttccccaagccacgcttctggcagagagccgggggccaagcccagcactgcagagcccagctcgagcccccaggaccctcccaagcctgttgctgcgcacagtgaaagcagcagccacaagccccggcctggccctgacccgggccctccaaagactaagcaccccgaccggtccctctcctctcagaaaccaagtgtcggggccacaaagggcaaagagcctgccactcagtccctcggtggctctagcagagaggggaagggccacagtaagagtgggccggatgtgtttcctgctaccccaggctcccagaacaaagccagcgatgggattggccagggagaaggtgggccctctgtcccactgcacactgacagggctcctctagacgccaagccacaacccaccagtggtgggcggcccctggaggtgctggagaagcctgtgcatttgccaaggccgggacacccagggcctagtgagccagcggaccagaaactgtccgctgttggtgaaaagcaaaccctgtctccaaagcaccccaaaccatccactgtgaaagattgccccaccctgtgcaaacagacagacaacagacagacagacaaaagcccgagtcagccggccgccaacaccgacagaagggcggaagggaagaaatgcactgaagcactttatgctccagcagagggcgacaagctcgaggccggcctttcctttgtgcatagcgagaaccggttgaaaggcgcggagcggccagccgcgggggtggggaagggcttccctgaggccagagggaaagggcccggtccccagaagccaccgacggaggcagacaagcccaatggcatgaaacggtccccctcagccactgggcagagttctttccgatccacggccctcccggaaaagtctctgagctgctcctccagcttccctgaaaccagggccggagttagagaggcctctgcagccagcagcgacacctcttctgccaaggccgccgggggcatgctggagcttccagcccccagcaacagggaccataggaaggctcagcctgccggggagggccgaacccacatgacaaagagtgactccctgccctccttccgggtctccaccctgcctctggagtcacaccaccccgacccaaacaccatgggcggggccagccaccgggacagggctctctcggtgactgccaccgtaggggaaaccaaagggaaggaccctgccccagcccagcctcccccagctaggaaacagaacgtgggcagagacgtgaccaagccatccccagccccaaacactgaccgccccatctctctttctaatgagaaggactttgtggtacggcagaggcgggggaaagagagtttgcgtagcagccctcacaaaaaggccttgtaacggggagggcccaggggcaggactgtggagacccgtcctgaacgggcgactgtgtcttgactacctttcaaaaccagcactgtgtgggaatgtccgccaggcagagctcggagcctcattgagacaggggagagagaaagacaaagaggggaccttcttccagatgccttcccagttgtaaccggtaaaactgttaccagatagtgtttgtac

SEQ ID NO: 4 - DNA, 981 bp, Homo sapiens
atgggagcgaacacttcaagaaaaccaccagtgtttgatgaaaatgaagatgtcaactttgaccactttgaaattttgcgagccattgggaaaggcagttttgggaaggtctgcattgtacagaagaatgataccaagaagatgtacgcaatgaagtacatgaataaacaaaagtgcgtggagcgcaatgaagtgagaaatgtcttcaaggaactccagatcatgcagggtctggagcaccctttcctggttaatttgtggtattccttccaagatgaggaagacatgttcatggtggtggacctcctgctgggtggagacctgcgttatcacctgcaacagaacgtccacttcaaggaagaaacagtgaagctcttcatctgtgagctggtcatggccctggactacctgcagaaccagcgcatcattcacagggatatgaagcctgacaatattttacttgacgaacatgggcacgtgcacatcacagatttcaacattgctgcgatgctgcccagggagacacagattaccaccatggctggcaccaagccttacatggcacctgagatgttcagctccagaaaaggagcaggctattcctttgctgttgactggtggtccctgggagtgacggcatatgaactgctgagaggccggagaccgtatcatattcgctccagtacttccagcaaggaaattgtacacacgtttgagacgactgttgtaacttacccttctgcctggtcacaggaaatggtgtcacttcttaaaaaggtaagaaggaagactgcatgtccaaacgaagtaacaaaaggaagcaggctctctggcttaagtttagaagttagtatacaatattggggacaccaactgtcttccttgcagaaaggcaggctgaattgtgatcctacctttgaacttgaggaaatgattttggagtccaaacctctacataagaaaaaaaagcgtctggcaaag

SEQ ID NO: 5 - DNA, 2260 bp, Homo sapiens
atctttgggctgcttgggcgaggcgacccttgggtcggcgttgcgggtgaggtgggcaggtaggtgggcggatggccgcggttctccgacaggcgcgggcggcggggtctcccacggcgtccgaagccccctccccgcacccccggcctccagcgttgaggcgggggagtgaggagatgccgacccagagggacagcagcaccatgtcccacagggttgcaggcggctgcagtggggaccattcccaccaggtctgggtgaaagcctactaccgcggggatatcatgataacacattttgaaccttctgtctcctttgagggcctttacaatgtggttcgagacatgtgttcttttgacaacgaacagctcttcaccatgaaatggatagacgaggaaggagacccgtgtacagtatcatctcagttggagttagaagaagcctttacactttatgagctaaacaaggactctgaactcttgattcatgtgttcccttgtgtaccagaacgtcccgggatgccttgtccaagagaagataaatccatctaccgtagaggtgcacgccactggagaaagctttattgtgccaatggacacacttttcaagccaagtgcttcaacaggcgtgctgactgtgccatctgcacagacagaatatggggacttggatgccaaggatataagtgtatcaactgcaaactcttggttcataagaagtgccataaactcctcacaattgaatgtgggcggcattctttgccactggaaccaatgatgcccatggatcagtcatccatgcattctgaccatgcacagacagtaattccatataatccttcaagtcatgagagtttggatcaagttggtgaagaaaaagaggcaatgaacaccagagaaagtggcaaagcttcatccagtctaggtcttcaggattttgatttgctccaggtaataggaagaggaagttatgccaaagtactgttggttcgattaaaaaaaacagatcgtatttatgcaatgaaagttgtgaaaaaagagcttgttaatgatgatgaggatattgattgggtgcagacagagaaacatgtgtttgagcaggcatccaatcatcctttccttgttgggctgcagtcttgctttcagacagaaagcagattgttctttgttatagagtatgtaaatggaggagacctaatgtttcatatgcagcgacaaagaaaacttcctgaagaacatgccagattttactctgcagaaatcagtctagcattcaattatcttcatgagagagggataatttatagagatttgaaactggacaatgtattactggactctgaaggccacattaaactcactgactacggcatgtgtaaggaaggattacggccaggagatacaaccagcactttctgtggtactcctaattacattgctcctgaaattttaagaggaggagattatggtttcagtgttgactggtgggctcttggagtgctaatgtttgagacgatggcaggaaggactccatttgatactgttgggagctccgataaccctgaccagaacacagaggattctctcttccaagttattgtggaaaaacaaattcacataccacgttctctgtctggaaaagctgcaagtgttctgaagagttttcttaacaaggaccctaagaaacgactgggttgtcatcctcaaacaggatttgctgatattcagggacacccgttcttccaaaatgttgattgggatatgatggagcaaaaacaggtggtatctccctttaaaccaagtatttctggggaatttggtttggacaactttgattctcagtttactaatgaacctgtctggctcactccagatgtcgatgacattgtgagaaagattgatcagtctgaatttgaaggttttgagtatatcaatcctcttttgatgtctgcagaagaatgtgtctgatcctcatttttcaaccatgtattctactcatgttgccatttaatgcatggataaacttgctgccagcctggatacaattaaccattttatatttgccacctacaaaaaaacacccagtatcttctctcgtagactacatgaatcaattattacatcttttttactatgaaaaataaattaatactagcttccagacaatcatgtcaaaatttagttgaactggtttttcagtttttaaaaggcctacagatgagtaatgaagttatcttttttgtttaaaaaaaaaacaaaga

SEQ ID NO: 6 - DNA, 1716 bp, Homo sapiens
ccttcccagttctgctcccacagagcaccacggattgtgattgtcccagctccgtccccctttctcagacccgtcaatcctaagagccaggcctggtacccatgggtctccttcctcaccaaggccagcccgcgccccgcagtcccatggcacctgccagacaggacacggagcctggcttgtggaggttcacgcctgcaggacggggacgaactagatccaaatccaagaccccagcacccagcggagggtccagaactggaaactgtgtttcctaagcaggaagagatgcccggcaaacagtctgaggaagggccggcggaggcaggggcttcggaggacagcgaggaggagggtctgggcggcctgacattagaggagctccagcagggccaggaggctgcccgcgcgctggaggacatgatgacgctgagtgctcagaccctggtccgagccgaggtggacgagctctacgaggaagtgcgtcccctgggccagggtcgctatggccgcgtccttctggtcacccatcgtcagaaaggcacacccctggcactgaagcagctcccgaaaccccgcacgtccctccgtggcttcctgtacgagttctgtgtggggctctcgctgggcgcgcactcagccatcgtgacggcctacggcattggcatcgagtcggcacactcctacagcttcctgacggagcccgtcctgcacggggacctcatggccttcatccagcccaaggtgggcctcccgcagcccgcggtgcaccgctgcgccgcccagctggcctccgccctggagtacatccacgcccgcggcctggtgtaccgggacctgaagccggagaacgtcctggtgtgcgacccggcctgccggcgcttcaagctgaccgacttcggccacacgaggcctcgcgggacgctgctgcgcctggccgggccgcccatcccctacacggcccccgagctctgcgcgcccccgccgctccccgagggcctgcccattcagcccgccctggacgcctgggcgctgggcgtcctgctcttctgcctcctcacgggctacttcccctgggaccggcccctggccgaggccgaccccttctacgaggacttcctcatctggcaggcgtcgggccagccccgggaccgccctcagccctggttcggcctggcccccgcggccgacgcgcttctgcgggggctgctggaccctcacccccgaaggaggagcgctgtgatcgccatcagggagcacctggggcgcccctggaggcagcgggagggcgaggcggaggcagtgggagcggtggaagaggaggctgggcaggcgcctgctgtggccaccgaggtgttgcctggagcggggctgacagcaggtgtgctcctgtggagtgaccccaaggagccttcgaatccaggttcaagccatagagttggggcctctgacctggacaatccggggatgtcctgccgtgctcagcgggaggaggggcttgggggcacgggggtgtgggagcagaccgtccatctatcaggacccccagacgttatccgcaccccgccacccccccgcccaagtaactggaccctgttggcctttgcaggactgcgggggcgccagccttcatcaccttccacgacctctaagccgagagaggaggaggaggaggaaatggtttcctgcctgaggctgcaggacccagaatcagag

SEQ ID NO: 7 - DNA, 1119 bp, Homo sapiens
atgcacccccactcggggctgggggctcctggcctgctcccacagacaggggcaggcggggcctctgtggcagtgacccctaacctctccaggacacaaaaacaggtggcccgagtccgggaggacacagccacagccctccaacggctggtggagctgacgaccagcagggtgaccccggtgaggagccttcgggaccagtaccacctcatccggaagctgggctccggctcctacggccgcgtgctcctcgcccagcctcaccaggggggtccagctgtggctctgaagctcctgcgtcgggatttggtcctgagaagcaccttcctgagggagttctgtgtgggccgctgcgtctctgcacacccaggcctgctgcagaccctggcaggacccctacagaccccccgctattttgccttcgcccaggagtacgcgccctgtggggacctcagcgggatgctgcaggaaaggggcctcccagaactgctggtgaagcgggtggtggcccagttggcaggagctctggacttcctccacagccgggggctggtccacgcagatgtcaaacctgacaacgtgctggtcttcgacccagtctgcagccgtgtggccctgggagacctgggtctgacccggccagagggcagcccgacccccgccccaccagtgcctctgcccacggcaccgcctgagctctgtctcctgctaccgcccgacaccctgcctctgcggccagccgtggactcctggggcctgggggtgcttctcttctgtgctgccactgcctgtttcccttgggacgtggcactggcccccaaccctgagttcgaggccttcgctggctgggtgaccaccaagcctcagccacctcagccaccaccaccctgggaccagtttgcgcccccagccctggccttgctccaggggcttctggacctggatcccgagactaggagccccccactggctgtcctggacttcctgggggacgactgggggttgcaagggaacagagagggacctggggttttggggagcgccgtgtcctatgaggacagggaggagggaggctcaagcctggaggagtggacagatgagggtgatgacagcaaaagt

SEQ ID NO: 8 - DNA, 1074 bp, Homo sapiens
atggcccgggagaacggcgagagcagctcctcctggaaaaagcaagctgaagacatcaagaagatcttcgagttcaaagagaccctcggaaccggggccttttccgaagtggttttagctgaagagaaggcaactggcaagctctttgctgtgaagtgtatccctaagaaggcgctgaagggcaaggaaagcagcatagagaatgagatagccgtcctgagaaagattaagcatgaaaatattgttgccctggaagacatttatgaaagcccaaatcacctgtacttggtcatgcagctggtgtccggtggagagctgtttgaccggatagtggagaaggggttttatacagagaaggatgccagcactctgatccgccaagtcttggacgccgtgtactatctccacagaatgggcatcgtccacagagacctcaagcccgaaaatctcttgtactacagtcaagatgaggagtccaaaataatgatcagtgactttggattgtcaaaaatggagggcaaaggagatgtgatgtccactgcctgtggaactccaggctatgtcgctcctgaagtcctcgcccagaaaccttacagcaaagccgttgactgctggtccatcggagtgattgcctacatcttgctctgcggctaccctcctttttatgatgaaaatgactccaagctctttgagcagatcctcaaggcggaatatgagtttgactctccctactgggatgacatctccgactctgcaaaagacttcattcggaacctgatggagaaggacccgaataaaagatacacgtgtgagcaggcagctcggcacccatggatcgctggtgacacagccctcaacaaaaacatccacgagtccgtcagcgcccagatccggaaaaactttgccaagagcaaatggagacaagcatttaatgccacggccgtcgtgagacatatgagaaaactacacctcggcagcagcctggacagttcaaatgcaagtgtttcgagcagcctcagtttggccagccaaaaagactgtgcgtatgtagcaaaaccagaatccctcagctga

SEQ ID NO: 9 - DNA, 1077 bp, Homo sapiens
atgcgagccacccctctggctgctcctgcgggttccctgtccaggaagaagcggttggagttggatgacaacttagataccgagcgtcccgtccagaaacgagctcgaagtgggccccagcccagactgcccccctgcctgttgcccctgagcccacctactgctccagatcgtgcaactgctgtggccactgcctcccgtcttgggccctatgtcctcctggagcccgaggagggcgggcgggcctaccaggccctgcactgccctacaggcactgagtatacctgcaaggtgtaccccgtccaggaagccctggccgtgctggagccctatgcgcggctgcccccgcacaagcatgtggctcggcccactgaggtcctggctggtacccagctcctctacgcctttttcactcggacccatggggacatgcacagcctggtgcgaagccgccaccgtatccctgagcctgaggctgccgtgctcttccgccagatggccaccgccctggcgcactgtcaccagcacggtctggtcctgcgtgatctcaagctgtgtcgctttgtcttcgctgaccgtgagaggaagaagctggtgctggagaacctggaggactcctgcgtgctgactgggccagatgattccctgtgggacaagcacgcgtgcccagcctacgtgggacctgagatactcagctcacgggcctcatactcgggcaaggcagccgatgtctggagcctgggcgtggcgctcttcaccatgctggccggccactaccccttccaggactcggagcctgtcctgctcttcggcaagatccgccgcggggcctacgccttgcctgcaggcctctcggcccctgcccgctgtctggttcgctgcctccttcgtcgggagccagctgaacggctcacagccacaggcatcctcctgcacccctggctgcgacaggacccgatgcccttagccccaacccgatcccatctctgggaggctgcccaggtggtccctgatggactggggctggacgaagccagggaagaggagggagacagagaagtggttctgtatggctag

SEQ ID NO: 10 - DNA, 1542 bp, Homo sapiens
atggaggggggtccagctgtctgctgccaggatcctcgggcagagctggtagaacgggtggcagccatcgatgtgactcacttggaggaggcagatggtggcccagagcctactagaaacggtgtggaccccccaccacgggccagagctgcctctgtgatccctggcagtacttcaagactgctcccagcccggcctagcctctcagccaggaagctttccctacaggagcggccagcaggaagctatctggaggcgcaggctgggccttatgccacggggcctgccagccacatctccccccgggcctggcggaggcccaccatcgagtcccaccacgtggccatctcagatgcagaggactgcgtgcagctgaaccagtacaagctgcagagtgagattggcaaggtggggctgactgatgcctatctgcagggtgcctacggtgtggtgaggctggcctacaacgaaagtgaagacagacactatgcaatgaaagtcctttccaaaaagaagttactgaagcagtatggctttccacgtcgccctcccccgagagggtcccaggctgcccagggaggaccagccaagcagctgctgcccctggagcgggtgtaccaggagattgccatcctgaagaagctggaccacgtgaatgtggtcaaactgatcgaggtcctggatgacccagctgaggacaacctctatttggttgacctcctgagaaaggggcccgtcatggaagtgccctgtgacaagcccttctcggaggagcaagctcgcctctacctgcgggacgtcatcctgggcctcgagtacttgcactgccagaagatcgtccacagggacatcaagccatccaacctgctcctgggggatgatgggcacgtgaagatcgccgactttggcgtcagcaaccagtttgaggggaacgacgctcagctgtccagcacggcgggaaccccagcattcatggcccccgaggccatttctgattccggccagagcttcagtgggaaggccttggatgtatgggccactggcgtcacgttgtactgctttgtctatgggaagtgcccattcatcgacgatttcatcctggccctccacaggaagatcaagaatgagcccgtggtgtttcctgaggagccagaaatcagcgaggagctcaaggacctgatcctgaagatgttagacaagaatcccgagacgagaattggggtgccagacatcaagttgcacccttgggtgaccaagaacggggaggagccccttccttcggaggaggagcactgcagcgtggtggaggtgacagaggaggaggttaagaactcagtcaggctcatccccagctggaccacggtgatcctggtgaagtccatgctgaggaagcgttcctttgggaacccgtttgagccccaagcacggagggaagagcgatccatgtctgctccaggaaacctactggtgaaagaagggtttggtgaagggggcaagagcccagagctccccggcgtccaggaagacgaggctgcatcctga

SEQ ID NO: 11 - DNA, 1032 bp, Homo sapiens
atgctgctgctgaagaaacacacggaggacatcagcagcgtctacgagatccgcgagaggctcggctcgggtgccttctccgaggtggtgctggcccaggagcggggctccgcacacctcgtggccctcaagtgcatccccaagaaggccctccggggcaaggaggccctggtggagaacgagatcgcagtgctccgtaggatcagtcaccccaacatcgtcgctctggaggatgtccacgagagcccttcccacctctacctggccatggaactggtgacgggtggcgagctgtttgaccgcatcatggagcgcggctcctacacagagaaggatgccagccatctggtgggtcaggtccttggcgccgtctcctacctgcacagcctggggatcgtgcaccgggacctcaagcccgaaaacctcctgtatgccacgccctttgaggactcgaagatcatggtctctgactttggactctccaaaatccaggctgggaacatgctaggcaccgcctgtgggacccctggatatgtggccccagagctcttggagcagaaaccctacgggaaggccgtagatgtgtgggccctgggcgtcatctcctacatcctgctgtgtgggtacccccccttctacgacgagagcgaccctgagctcttcagccagatcctgagggccagctatgagtttgactctcctttctggggtgacatctcagaatcagccaaagacttcatccggcaccttctggagcgagacccccagaagaggttcacctgccaacaggccttgcggcacctttgggtctctggggacacagccttcgacagggacatcttaggctctgtcagtgagcagatccggaagaactttgctcggacacactggaagcgagccttcaatgccacctcgttcctgcgccacatccggaagctggggcagatcccagagggcgagggggcctctgagcagggcatggcccgccacagccactcaggcctccgtgctggccagccccccaagtggtga

SEQ ID NO: 12 - DNA, 1500 bp, Homo sapiens
atggcttcgaccaccacctgcaccaggttcacggacgagtatcagcttttcgaggagcttggaaagggggcattctcagtggtgagaagatgtatgaaaattcctactggacaaggatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagcagccaagagtttgttgaagaaaccagatggagtaaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagccaccctgtattccaaatgggaaagaaaacttctcaggaggcacctctttgtggcaaaacatctga

SEQ ID NO: 13 - DNA, 1311 bp, Homo sapiens
atgactgcagtgtatatgaatggaggtggcctggtgaacccccactatgcccggtgggatcggcgcgacagtgtagaaagtggctgtcagaccgagagtagcaaggagggtgaggagggacagccccgccagctgacgcccttcgagaaactgacacaggacatgtcccaggatgagaaggtggtgagggagatcacgctggggaaacggataggcttctaccgaattcgaggggaaatcggaagtggaaacttctcccaagtgaagcttgggattcactccctaaccaaagaaaaggtggccattaagatcctggacaagaccaagttagaccagaaaacccagaggctactatcccgagaaatctccagcatggaaaagctgcaccatcccaacatcatccgcctttacgaagtggtggagaccctatccaagctgcacttggtgatggagtatgcagggggtggggagctcttcggaaaaattagcactgaggggaagctctctgaaccagaaagcaagctcatcttctcccagattgtgtctgccgtgaagcacatgcatgaaaaccaaattattcatagagatctgaaagcagaaaatgtattctataccagtaatacttgtgtgaaggtgggcgattttggattcagcacagtaagcaaaaaaggtgaaatgctgaacactttctgtgggtctcctccctacgctgcgcctgaactcttccgggacgagcactacatcggcatttacgtggatatctgggccttgggggtgcttttgtacttcatggtgactggcaccatgccatttcgggcagaaaccgtggccaaactaaaaaagagcatcctcgagggcacatacagtgtaccgccgcacgtgtcagagccctgccaccgactcatccgaggagtccttcagcagatccccacggagaggtacggaatcgactgcatcatgaatgatgaatggatgcaaggggtgccataccctacacctttggaacctttccaactggatcccaaacatttgtcggaaaccagcactctcaaggaagaagaaaatgaggtcaaaagcactttagaacatttgggcattacagaagagcatattcgaaataaccaagggagagatgctcgcagctcaatcacaggggtctatagaattattttacatagagtccaaaggaagaaggctttggaaagtgtcccagtcatgatgctaccagaccctaaagaaagagacctcaaaaaagggtcccgtgtctacagagggataagacacacatccaaattttgctcgattttataa

SEQ ID NO: 14 - DNA, 225 bp, Homo sapiens
gaagaggaggaggcccagaccatgtttgggcaaattctgttagcaatgtagtactgcctctataaaaacgtcgcacatgcagacgtaagcttacaaatcatcctactccctgaagagcgcaatataaaaatcgtggactttggctttagcatgacattcggggaaaggagattcggaaaccctttttgtggcatgtacccctatgtggccacagaactcttcctg

SEQ ID NO: 15 - DNA, 117 bp, Homo sapiens
ttcaggcagattctgtcaggcctgcagtactgccgcttaaagaacatcacacatccagagctaaagccacaaacccatctagtcatagaggaaggcaatataaaaataacggatttt

SEQ ID NO: 16 - DNA, 252 bp, Homo sapiens
aatatcttctttgacaaagaacgtaacatcaaaatagccaactatgggtttagttcaacatttgcaaagagagacaacactgaaaagggaacgtttgatgggactctcccttatgcaggcccagaactcttcttgggccatgggtaccaatgccctgccatcaacgtttggagccttggcatcatcgtatgcaaaatggtggctggggccctgccattttatgagcagaactgtaaggatatactgaaaaaa

SEQ ID NO: 17 - DNA, 2385 bp, Homo sapiens
atggtggcggggttaactttggggaagggcccggagtccccggatggtgatgtcagcgtgccggagagaaaggacgaggtggcggggggaggcggagaggaggaggaggccgaagagagagggcgccacgcccaatatgtgggcccctatcggctggagaagacgctgggcaaaggacagacagggctggttaaactcggggtccactgcatcacgggtcagaaggtcgccatcaagatcgtgaaccgggagaagctgtcggagtcggtgctgatgaaggtggagcgggagatcgccatcctgaagctcatcgaacacccacatgtcctcaagctccacgacgtctacgagaacaagaaatatttgtacctggttctggagcacgtctcggggggtgagctattcgactacctggtaaagaaggggagactgacgcccaaggaggcccgaaagttcttccgccagattgtgtctgcgctggacttctgccacagctactccatctgccacagagacctaaagcccgagaacctgcttttggatgagaaaaacaacatccgcattgcagacttcggcatggcgtccctgcaggtgggggacagcctcctggagaccagctgcgggtccccccattatgcgtgtccagaggtgattaagggggaaaaatatgatggccgccgggcagacatgtggagctgtggagtcatcctcttcgccctgctcgtgggggctctgccctttgatgacgacaacctccgccagctgctggagaaggtgaaacggggcgtcttccacatgccccacttcattcctccagattgccagagcctcctgaggggaatgatcgaagtggagcccgaaaaaaggctcagtctggagcaaattcagaaacatccttggtacctaggcgggaaacacgagccagacccgtgcctggagccagcccctggccgccgggtagccatgcggagcctgccatccaacggagagctggaccccgacgtcctagagagcatggcatcactgggctgcttcagggaccgcgagaggctgcatcgcgagctgcgcagtgaggaggagaaccaagaaaagatgatatattatctgcttttggatcggaaggagcggtatcccagctgtgaggaccaggacctgcctccccggaatgatgttgaccccccccggaagcgtgtggattctcccatgctgagccgtcacgggaagcggcgaccagagcggaagtccatggaagtcctgagcatcaccgatgccgggggtggtggctcccctgtacccacccgacgggccttggagatggcccagcacagccagagatcccgtagcgtcagtggagcctccacgggtctgtcctccagccctctaagcagcccaaggagtccggtcttttccttttcaccggagccgggggctggagatgaggctcgaggcgggggctccccgacttccaaaacgcagacgctgccttctcggggccccaggggtgggggcgccggggagcagcccccgccccccagtgcccgctccacacccctgcccggccccccaggctccccgcgctcctctggcgggacccccttgcactcgcctctgcacacgccccgggccagtcccaccgggaccccggggacaacaccaccccccagccccggcggtggcgtcgggggagccgcctggaggagtcgtctcaactccatccgcaacagcttcctgggctcccctcgctttcaccggcgcaagatgcaggtccctaccgctgaggagatgtccagcttgacgccagagtcctccccggagctggcaaaacgctcctggttcgggaacttcatctccttggacaaagaagaacaaatattcctcgtgctaaaggacaaacctctcagcagcatcaaagcagacatcgtccatgcctttctgtcgatccccagcctgagtcacagtgtgctgtcacagaccagcttcagggccgagtacaaggccagtggcggcccctccgtcttccaaaagcccgtccgcttccaggtggacatcagctcctctgagggtccagagccctccccgcgacgggacggcagcggaggtggtggcatctactccgtcaccttcactctcatctcgggtcccagccgtcggttcaagcgagtggtggagaccatccaggcacagctcctgagcactcatgaccagccctccgtgcaggccctggcagacgagaagaacggggcccagacccggcctgctggtgccccaccccgaagcctgcagcccccacccggccgcccagacccagagctgagcagctctccccgccgaggcccccccaaggacaagaagctcctggccaccaacgggacccctctgccctga

SEQ ID NO: 18 - DNA, 2361 bp, Homo sapiens
atggttatcatgtcggagttcagcgcggaccccgcgggccagggtcagggccagcagaagcccctccgggtgggtttttacgacatcgagcggaccctgggcaaaggcaacttcgcggtggtgaagctggcgcggcatcgagtcaccaaaacgcaggttgcaataaaaataattgataaaacacgattagattcaagcaatttggagaaaatctatcgtgaggttcagctgatgaagcttctgaaccatccacacatcataaagctttaccaggttatggaaacaaaggacatgctttacatcgtcactgaatttgctaaaaatggagaaatgtttgattatttgacttccaacgggcacctgagtgagaacgaggcgcggaagaagttctggcaaatcctgtcggccgtggagtactgtcacgaccatcacatcgtccaccgggacctcaagaccgagaacctcctgctggatggcaacatggacatcaagctggcaggcacggaggattttggatttgggaatttctacaagtcaggagagcctctgtccacgtggtgtgggagccccccgtatgccgccccggaagtctttgaggggaaggagtatgaaggcccccagctggacatctggagcctgggcgtggtgctgtacgtcctggtctgcggttctctccccttcgatgggcctaacctgccgacgctgagacagcgggtgctggagggccgcttccgcatccccttcttcatgtctcaagactgtgagagcctgatccgccgcatgctggtggtggaccccgccaggcgcatcaccatcgcccagatccggcagcaccggtggatgcgggctgagccctgcttgccgggacccgcctgccccgccttctccgcacacagctacacctccaacctgggcgactacgatgagcaggcgctgggtatcatgcagaccctgggcgtggaccggcagaggacggtggagtcactgcaaaacagcagctataaccactttgctgccatttattacctcctccttgagcggctcaaggagtatcggaatgcccagtgcgcccgccccgggcctgccaggcagccgcggcctcggagctcggacctcagtggtttggaggtgcctcaggaaggtctttccaccgaccctttccgacctgccttgctgtgcccgcagccgcagaccttggtgcagtccgtcctccaggccgagatggactgtgagctccagagctcgctgcagtggcccttgttcttcccggtggatgccagctgcagcggagtgttccggccccggcccgtgtccccaagcagcctgctggacacagccatcagtgaggaggccaggcaggggccgggcctagaggaggagcaggacacgcaggagtccctgcccagcagcacgggccggaggcacaccctggccgaggtctccacccgcctctccccactcaccgcgccatgtatagtcgtctccccctccaccacggcaagtcctgcagagggaaccagctctgacagttgtctgaccttctctgcgagcaaaagccccgcggggctcagtggcaccccggccactcaggggctgctgggcgcctgctccccggtcaggctggcctcgcccttcctggggtcgcagtccgccaccccagtgctgcaggctcaggggggcttgggaggagctgttctgctccctgtcagcttccaggagggacggcgggcgtcggacacctcactgactcaagggctgaaggcctttcggcagcagctgaggaagaccacgcggaccaaagggtttctgggactgaacaaaatcaaggggctggctcgccaggtgtgccaggcccccgccagccgggccagcaggggcggcctgagccccttccacgcccctgcacagagcccaggcctgcacggcggcgcagccggcagccgggagggctggagcctgctggaggaggtgctagagcagcagaggctgctccagttacagcaccacccggccgctgcacccggctgctcccaggccccccagccggcccctgccccgtttgtgatcgccccctgtgatggccctggggctgccccgctccccagcaccctcctcacgtcggggctcccgctgctgccgcccccactcctgcagaccggcgcgtccccggtggcctcagcggcgcagctcctggacacacacctgcacattggcaccggccccaccgccctccccgctgtgcccccaccacgcctggccaggctggccccaggttgtgagcccctggggctgctgcagggggactgtgagatggaggacctgatgccctgctccctaggcacgtttgtcctggtgcagtga

SEQ ID NO: 19 - DNA, 789 bp, Homo sapiens
gtgcctctgtatttcaggaagctctcccatgatctgcaaatgactttcccttacactaagaaagttggggcttatttggttggcaaaatgatcaacaagggtccttttgccaaagtcatagaagggctgggtctccccatgggtgaaaaggcaaccatcaaaggcattagcaagaagaaagccaagcaagactcctatgttctaaagaatataaagtgtgaatcttggatccagtaaattgtcaaataccctaatgcttcccagctacaaaccctggagactaagaactcttattatgtgatgatggagctatgcctcagtggcaacctcatgaacaggatctgggatcaaaagaaactaagtgaaagggaagtgaagatgatcttcaggcagatgttgtccgcaatggagcatgtgcactggcatggaatggtacacaaggattatgcattttttttcttctcaggcaggaaaagccaccaaagcccccaagtttatgaaacccttgacattaatactgagaagttggagggtctttctcaggagcagttaagtactcagggtggaagtcctgcctatgcggcccctgaactgcttgctcatcagaaatatggccccaaagtggatgtctggttcatgggtaagtttctgatgctgactggcaccttaccgttcacattggagatattcagtattgagcaactctatcagaagatggtgattggtgcaatcagcaatgtcctacctgaaatctcctttggtaatttactgtggtctctaagtttggta

SEQ ID NO: 20 - DNA, 2298 bp, Homo sapiens
atggcaggatttaagcgagggtatgatggaaagattgctggattatatgatctggataaaaccttgggtcgaggccattttgcctgtggtacttgctcgaggcatgtctttacgggtgaaaaggtggcagtaaaagttattgacaagacaaaactggacactctagctactggtcatcttttccaggaagtgagatgcatgaaactagtgcagcatcctaacatcgtccgcctttatgaagttattgacacccagaccaaactatatcttattctagaacttggggatggaggagatatgtttgattatataatgaaacatgaggagggtcttaatgaagacttggccaagaagtattttgctcagatagttcatgctatatcttattgccataaactccatgtggttcacagagacttaaaaccagagaatgtagtcttctttgaaaaacaaggtcttgtaaagttgacagactttgggttcagcaacaaatttcaaccagggaagaagctcactacaagctgtggatctcttgcatattccgctccagaaattctgcttggtgatgagtatgatgcacctgcagtagatatttggagtctgggagtgatccttttcatgttggtgtgtgggcagccgccctttcaagaagccaatgacagtgaaacactgacaatgatcatggattgcaaatatacagtaccatcccatgtgtctaaagagtgtaaagacctaatcacacggatgctacagagagatcccaagagaagggcttctttagaagagattgaaaatcatccttggcttcagggagtggacccttcaccagctacaaagtataacattccccttgtgtcatacaaaaatctctcggaagaggagcacaacagcatcattcagcgcatggtgcttggggacatagcggatcgagacgccattgtagaagccctggaaaccaacaggtataaccatatcacagccacatacttcctgctggctgaaaggatcctgagagaaaagcaagagaaagaaatacagaccagatctgcaagcccgagcaatatcaaggcccagtttaggcagtcatggccaaccaaaattgatgtaccccaggaccttgaggatgacctcacggccactcctttgtcccacgcgactgtccctcagtctcctgctcgggctgctgacagtgtcctcaatggccacaggagcaaaggcctgtgtgactcagctaagaaagatgacctccctgagttggctggaccagcactctctacggtgccacccgcaagcttaaaacccacagccagtgggcggaagtgtctgttcagggtggaagaagatgaagaggaagatgaggaggacaagaaacccatgtccctctcaacacaagtggttttgcgccggaagccatctgtaaccatccgcctgacatccaggaagagtgcgcccgtcctcaaccagatctttgaggaaggggaatctgacgatgagtttgacatggatgagaatctgcctcccaagttgagcaggttaaagatgaatatagcttctccaggtacagttcacaaacgctaccaccggaggaaaagtcagggccggggctccagctgcagtagttcggagaccagtgatgatgattctgaaagccggcggcggctcgataaagatagcgggttcacctactcctggcaccgacgggatagcagcgaggggccccctggcagtgagggggatggcgggggccagagcaagccgagcaatgccagtggaggggtggacaaggccagccccagtgagaacaatgctggtgggggcagtccctccagcggctcgggtggcaaccccaccaatacatcgggtaccacacgccgctgtgccggccccagcaactccatgcagctggcctctcgcagtgctggggagctcgttgagagcctcaaactcatgagcctctgcctcggctcccagcttcatgggagcaccaagtacattattgatccacagaatggcttgtcattttccagtgtgaaagtccaagagaaatctacgtggaaaatgtgcattagctccacagggaatgcagggcaggtccctgcagtgggcggcataaagtttttctctgaccacatggcagataccaccactgaattggaacggataaagagcaagaacctgaaaaataacgtgctgcagctacctctgtgcgaaaagaccatctctgtgaacatccagcggaaccctaaggaggggctgctgtgcgcatccagcccagccagctgttgccatgtcatctga

SEQ ID NO: 21 - DNA, 1836 bp, Homo sapiens
atggcgacagaaaatggagcagttgagctgggaattcagaacccatcaacagggaaggaaaggagggtggatcctgatgggtgtctcacctcctctgcagacaaggcacctaaaggtcccacaggtgaaagacccctggctgcagggaaagaccctggccccccagacccaaagaaagctccggatccacccaccctgaagaaagatgccaaagcccctgcctcagagaaaggggatggtaccctggcccaaccctcaactagcagccaaggccccaaaggagagggtgacaggggcggggggcccgcggagggcagtgctgggcccccggcagccctgccccagcagactgcgacacctgagaccagcgtcaagaagcccaaggctgagcagggagcctcaggcagccaggatcctggaaagcccagggtgggcaagaaggcagcagagggccaagcagcagccaggaggggctcacctgcctttctgcatagccccagctgtcctgccatcatctccagttctgagaagctgctggccaagaagcccccaagcgaggcatcagagctcacctttgaaggggtgcccatgacccacagccccacggatcccaggccagccaaggcagaagaaggaaagaacatcctggcagagagccagaaggaagtgggagagaaaaccccaggccaggctggccaggctaagatgcaaggggacacctcgagggggattgagttccaggctgttccctcagagaaatccgaggtggggcaggccctctgtctcacagccagggaggaggactgcttccagattttggatgattgcccgccacctccggcccccttccctcaccgcatggtggagctgaggaccgggaatgtcagcagtgaattcagtatgaactccaaggaggcgctcggaggtggcaagtttggggcagtctgtacctgcatggagaaagccacaggcctcaagctggcagccaaggtcatcaagaaacagactcccaaagacaaggaaatggtgttgctggagattgaggtcatgaaccagctgaaccaccgcaatctgatccagctgtatgcagccatcgagactccgcatgagatcgtcctgttcatggagtacatcgagggcggagagctcttcgagaggattgtggatgaggactaccatctgaccgaggtggacaccatggtgtttgtcaggcagatctgtgacgggatcctcttcatgcacaagatgagggttttgcacctggacctcaagccagagaacatcctgtgtgtcaacaccaccgggcatttggtgaagatcattgactttggcctggcacggaggtataaccccaacgagaagctgaaggtgaactttgggaccccagagttcctgtcacctgaggtggtgaattatgaccaaatctccgataagacagacatgtggagtatgggggtgatcacctacatgctgctgagcggcctctcccccttcctgggagatgatgacacagagaccctaaacaacgttctatctggcaactggtactttgatgaagagacctttgaggccgtatcagacgaggccaaagactttgtctccaacctcatcgtcaaggaccagagggcccggatgaacgctgcccagtgtctcgcccatccctggctcaacaacctggcggagaaagccaaacgctgtaaccgacgccttaagtcccagatcttgcttaagaaatacctcatgaagaggcgctggaagaaaaacttcattgctgtcagcgctgccaaccgcttcaagaagatcagcagctcgggggcactgatggctctgggggtc

SEQ ID NO: 22 - DNA, 1014 bp, Homo sapiens
atgacaaacaacagcggctccaaagccgaactcgttgtgggagggaaatacaaactggtgcggaagatcgggtctggctcctttggagacgtttatctgggcatcaccaccaccaacggcgaggacgtagcagtgaagctggaatctcagaaggtcaagcacccccagttgctgtatgagagcaaactctacacgattcttcaaggtggggttggcatcccccacatgcactggtatggtcaggaaaaagacaacaatgtgctagtcatggaccttctgggacccagcctcgaagacctctttaatttctgttcaagaaggttcaccatgaaaactgtacttatgttagccgaccagatgatcagcagaattgaatacgtgcatacaaagaattttctacaccgagacattaaaccagataacttcctgatgggtactgggcgtcactgtaataagttgttccttattgattttggtttggccaaaaagtacagagacaacaggaccaggcaacacataccgtacagagaagataaacacctcattggcactgtccgatatgccagcatcaatgcacatcttggtattgagcagagccgccgagatgacatggaatccttaggctacgttttcatgtattttaatagaaccagcctgccgtggcaaggactaagggctatgacaaaaaaacaaaaatatgaaaagattagtgagaagaagatgtccacccctgttgaagttttatgtaaggggtttcctgcagaattcgccatgtacttgaactactgtcgtgggctgcgctttgaggaagtcccagattacatgtatctgaggcagctattccgcattcttttcaggaccctgaaccaccaatatgactacacatttgattggacgatgttaaagcagaaagcagcacagcaggcagcctcttccagtgggcagggtcagcaggcccaaacccagacaggcaagcaaactgaaaaaaacaagaataatgtgaaagataactaa

SEQ ID NO: 23 - DNA, 1200 bp, Homo sapiens
  modified_base (1)..(1200): “N” represents a, t, c, g, other or unknown
atgagtgggggaggagagcagccggatatcctgagtgttggaatcctagtgaaagaaagatggaaagtgttgagaaagattgggggtgggggctttggagaaatttacgatgccttggacatgctcaccagggaaaatgttgcactgaaggtggaatcagctcaannncaaaaacaagttctgaaaatggaagttgctgttttgaaaaagctgcaaggtggctttgttcaaattaggatttatatacaaggcaggcacattgcattatattttgttgccatgtcacagtttggtcggaatctggcagatcttcgccgtagccagtcccgaggcacattcaccattagtaccactctccggctgggtagacagattttggagtctattgaaagcattcattctgtgggattcttgcatcgagacatcaaaccgtcgaacttcgctatgggtcgctttcctagtacatgtaggaaatgttacatgcttgattttggcttggctcgacaatttaccaattcctgtggtgacgtcagaccacctcgagctgtggcaggttttcgagggacagttcgttatgcatcaatcaacgcacatcggaacagggaaatgggaagacatgatgacctttggtccttattctacatgttggtggagtttgtggttggtcagctgccctggagaaaaataaaggacaaggagcaagtaggctctattaaggagagatatgaccacaggctcatgttgaaacatctccctccagaattcagcatctttctagaccatatctcttctttggattattttacaaaaccagactaccagcttcttacatccgtgtttgacaatagcatcaagacttttggagtaattgagagtgacccttttgactgggagaagactggaaatgatggctccctaacaaccaccactacttctaccacccctcagttgcacactcgcttgacccctgctgcaattggaattgccaatgctactcccatccctggagacttgcttcgagaaaatacagatgaggtatttccagatgaacagcttagcgatggagaaaatggcatccctgttggtgtgtcaccagataaattgcctggatctctgggacacccccgtccccaggagaaggatgtttgggaagagatggatgccaacaaaaacaagataaagcttggaatttgtaaggta

SEQ ID NO: 24 - DNA, 1773 bp, Homo sapiens
atggagatgtatgaaacccttggaaaagtgggagagggaagttacggaacagtcatgaaatgtaaacataagaatactgggcagatagtggccattaagatattttatgagagaccagaacaatctgtcaacaaaattgcgatgagagaaataaagtttctaaagcaatttcatcacgaaaacctggtcaatctgattgaagtttttagacagaaaaagaaaattcatttggtatttgaatttattgaccacacagtattagatgagttacaacattattgtcatggactagagagtaagcgacttagaaaatacctcttccagatccttcgagcaattgactatcttcacagtaataatatcattcatcgagatataaaacctgagaatattttagtatcccagtcaggaattactaagctctgtgattttggttttgcacgaacactagcagctcctggggacatttatacggactatgtggccacacgctggtatagagctcccgaattagtattaaaagatacttcttatggaaaacctgtggatatctgggctttgggctgtatgatcattgagatggccactggaaatccctatcttcctagtagttctgatttggatttactccataaaattgttttgaaagtgggcaatttgtcacctcacttgcagaatatcttttccaagagccccatttttgctggggtagttcttcctcaagttcaacaccccaaaaatgcaagaaaaaaatatccaaagcttaatggattgttggcagatatagttcatgcttgtttacaaattgatcctgctgacaggatatcatctagtgatcttttgcatcatgagtattttactagagatggatttattgaaaaattcatgccagaactgaaagctaaattactgcaggaagcaaaagtcaattcattaataaagccaaaagagagttctaaagaaaatgaactcaggaaagatgaaagaaaaacagtttataccaatacactgctaagtagttcagttttgggaaaggaaatagaaaaagagaaaaagcccaaggagatcaaagtcagagttattaaagtcaaaggaggaagaggagatatctcagaaccaaaaaagaaagagtatgaaggtggacttggtcaacaggatgcaaatgaaaatgttcatcctatgtctccagatacaaaacttgtaaccattgaaccaccaaaccctatcaatcccagcactaactgtaatggcttgaaagaaaatccacattgcggaggttctgtgacaatgccacccatcaatctaactaacagtaatttgatggctgcaaatctcagttcaaatctctttcaccccagtgtgaggttaactgaaagagcaaaaaagagacgcacttcttcacaatctattggacaagttatgcctaatagcaggcaagaggatccaggtcctattcaaagccaaatggagaagggtatatttaatgagcgaacaggtcacagtgaccaaatggcaaatgagaacaaaaggaagctgaatttttccagatctgacaggaaagaattccattttccagaattgcctgtcacaatacagtcaaaagatacaaaaggaatggaagttaaacagataaaaatgctgaagagggagtcaaagaaaacagagtcatctaagataccaactttacttaacgtggatcaaaatcaagaaaaacaagagggtggagatggccattgcgaggggaagaatttgaagagaaacaggttttttttc

SEQ ID NO: 25 - DNA, 1083 bp, Homo sapiens
atggaaaagtatgaaaaattagctaagactggagaagggtcttatggggttgtattcaaatgcagaaacaaaacctctggacaagtagtagctgttaaaaaatttgtggaatctgaagatgatcctgttgttaagaaaatagcactaagagaaatacgtatgttgaagcaattaaaacatccaaatcttgtgaacctcatcgaggtgttcaggagaaaaaggaaaatgcatttagtttttgaatactgtgatcatacacttttaaatgagctggaaagaaacccaaatggagttgctgatggagtgatcaaaagcgtattatggcaaacacttcaagctcttaatttctgtcatatacataactgtattcacagagatataaaacctgaaaatattctaataactaagcaaggaataatcaagatttgtgacttcgggtttgcacaaattctgagtaattcacatgttgggagaattgacttacctgatctgattgatgcatttgcagttccaggagatgcctacaccgattatgtagctacgagatggtaccgagctcctgaacttcttgtgggagatactcagtatggttcttcagtcgatatatgggctattggttgtgtttttgcagagctcctgacaggccagccactgtggcctggaaaatcagatgtggaccaactttatctgataatcagaacactaggaaaattaatcccaagacatcaatcaatctttaaaagtaacgggtttttccatggcatcagtatacctgagccagaagacatggaaactcttgaggaaaagttctcagatgttcatcctgtggctctgaacttcatgaaggggtgtctgaagatgaatccagatgacagattaacctgttcccaactcctggagagctcctactttgattcttttcaagaggcccaaattaaaagaaaagcacgtaatgaaggaagaaacagaagacgccaacagaatcaactgttgcctctcataccaggaagccacatctcccccacacctgatggaagaaaacaagtcctccagttaaaatttgatcaccttccaaacatttag

SEQ ID NO: 26 - DNA, 1677 bp, Homo sapiens
atgtgcaccgtagtggaccctcgcattgtccggagatacctactcaggcggcagctcgggcagggggcctatggcattgtgtggaaggcagtggaccggaggactggtgaggtcgtggccatcaagaaaatctttgatgcttttagggataagacagatgcccaggacatgggcttccttcttgctccacccacccacacacctgtgtttctgtctcttcagagaacattccgggaaatcacgctcctccaggagtttggggaccatcccaacatcatcagcctccttgacgtgatccgggcagagaacgacagggacatttacctggtgtttgagtttatggacactgacctgaacgcagtcatccggaagggcggcctgctgcaggacgtccacgtgcgctccatcttctaccagctcctgcgggccacccggttcctccactcggggcacgttgtgcaccgggaccagaagccgtccaatgtgctcctggatgccaactgcacagtgaagctgtgtgactttggcctggcccgctccctgggcgacctccctgaggggcctgaggaccaggccgtgacagagtacgtggccacacgctggtaccgagcaccggaggtgctgctctcttcgcaccgatacacccttggggtggacatgtggagtctgggctgtatcctgggggagatgctgcgggggagacccctgttccccggcacgtccaccctccaccagctggagctgatcctggagaccatcccaccgccatctgaggaggacctcctggctctcggctcaggctgccgtgcctctgtgctgcaccagctggggtcccggccacgacagacgctggatgccctcctaccgccagacacctccccagaggccttggacctccttaggcgactcctggtgttcgccccggacaagcggttaagcgcgacccaggcactgcagcacccctacgtgcagaggttccactgccccagcgacgagtgggcacgagaggcagatgtgcggccccgggcacacgaaggggtccagctctctgtgcctgagtaccgcagccgcgtctatcagatgatcctggagtgtggaggcagcagcggcacctcgagagagaagggcccggagggtgtctccccaagccaggcacacctgcacaaacccagagccgaccctcagctgccttctaggacacctgtgcagggtcccagacccaggccccagagcagcccaggccatgaccctgccgagcacgccaagaacgttcccaggcagaactccgctcccctgctccaaactgctctcctagggaatggggaaaggccccctggggcgaaggaagcgccccccttgacactctcgctggtgaagccaagcgggaggggagctgcgccctccctgacctcccaggctgcggctcaggtggccaaccaggccctgatccggggtgactggaaccggggcggtggggtgagggtggccagcgtacaacaggtccctccccggcttcctccggaggcccggcccggccggaggatgttcagcacctctgccttgcagggtgcccaggggggtgccagggctttgcttggaggctactcccaagcctacgggactgtctgccactcggcactgggccacctgcccctgctggaggggcaccatgtgtga

SEQ ID NO: 27 - DNA, 2256 bp, Homo sapiens
atggctgccatattgggagacaccatcatggtggctaaaggccttgtcaagctgacccaggcggccgtggaaacccacctgcagcacttgggcatcggaggggagctgatcatggcagccagggccctgcagtccacggctgtggagcagattggcatgttcttggggaaggtgcagggtcaggataaacatgaagaatattttgctgagaacttcggcggcccagaaggggagttccacttctcagtcccgcatgcagccggagcctccacagacttctcttcagcctccgctcccgaccagtcagcgcccccatccctgggtcatgcccacagcgagggcccagctcctgcctacgtggccagtggaccctttagagaagccgggttccccggccaggcctcctcccctctgggcagggccaacgggaggctctttgcaaaccccagagactcattctctgccatgggctttcagcgaaggttcttccaccaggaccaatcccctgttgggggcctcacagccgaggacattgagaaggcccggcaggctaaggctcgccccgagaacaagcagcacaaacagacgctcagcgagcatgcccgggagcggaaggtgcctgtgacgaggattggccggctggccaacttcggaggtctggccgtgggcctgggcttcggggcactggcagaggtcgccaagaagagcctgcgctccgaggacccctcagggaagaaggccgtgctgggttccagtcctttcctgtccgaggccaatgcagagcggatcgtgcgcacgctctgcaaggtgcgtggtgcggcactcaagctgggccagatgctgagcatccaggatgatgcctttatcaacccccacccggcgagtgctctactcacgcccgcacgacagcctgtgaggcaggggctgcccagactgctggggacgtggagggtgcactgttgcccaggtctggccgtgggcctgggcttcggggcactggcagaggtcgccaagaagagcctgcgctccgaggacccctcagggaagaaggccgtgctgggttccagtcctttcctgtccgaggccaatgcagagcggatcgtgcgcacgctctgcaaggtgcgtggtgcggcactcaagctgggccagatgctgagcatccaggatgatgcctttatcaacccccacctggctaagatcttcgagcgggtgcggcagagcgcggacttcatgccactgaagcagatgatgaaaactctcaacaacgacctgggccccaactggcgggacaagttggaatacttcgaggagcggcccttcgccgccgcatccattgggcaggtgcacttggcccgaatgaagggcggccgcgaggtggccatgaagatccagtaccctggcgtggcccagagcatcaacagtgatgtcaacaacctcatggccgtgttgaacatgagcaacatgcttccagaaggcctgttccccgagcacctgatcgacgtgctgaggcgggagctggccctggagtgtgactaccagcgagaggccgcctgtgcccgcaagttcagggacctgctgaagggccaccccttcttctatgtgcctgagattgtggatgagctctgcagcccacatgtgctgaccacagagctggtgtctggcttccccctggaccaggccgaagggctcagccaggagattcggaacgagatctgctacaacatcctggttctgtgcctgagggagctgttcgagttccacttcatgcaaacagaccccaactggtccaacttcttctatgacccccagcagcacaaggtggctcttttggattttggggcaacgcgggaatatgacagatccttcaccgacctctacattcagatcatcagggctgctgccgacagggacagggagactgtgcgggcgaaatccatagagatgaagttcctcaccggctacgaggtcaaggtcatggaagacgcccacttggatgccatcctcatcctgggggaggccttcgcctctgatgagccttttgattttggcactcagagcaccaccgagaagatccacaacctgattcccgtcatgctgaggcaccgtctcgtccccccacccgaggaaacctactccctgcacaggaagatggggggctccttcctcatctgctccaagctgaaggcccgcttcccctgcaaggccatgttcgaggaggcctacagcaactactgcaagaggcaggcccagcagtag

SEQ ID NO: 28 - DNA, 1881 bp, Homo sapiens
atggtggcgccctggcgcgtctccgtcagggtttgcctgtcgcacctgaggtgcttcgagctcagacagggactcagcctcctgaggccctccgagtgccctcgcgatgccaggctctgctggcttctgctgggcactttgcccaaggtcgtctccctgtgcggggacgtgggtgagggggcccctgacgttctgagtcggcgaagggtccgctgcagcggggcggctggcgcggggcccgcggagagcctcccccgagcgggacctctgggcggcgtcttcctgcatctccgcctctggcttcgcgccggcgctctgttggtgaaattcttccccctcctactcctctaccccctcacctacctggctcccagcgtctccaccctctggctccacctgcttctgaaagccaccgagacctcaggcccaacctacatcaaactgggccagtgggccagcacccggcgcgatctgttttcggaggctttctgtgcccaattttccaagctgcatgtccgagtgacgccccacccgtggactcacactgagcgcttccttcggcaggcttttggggatgactgggggagcatcctctcttttgagaaccgggaacctgtgggctcaggctgcgtggcccaggtgtacaaagcatacgccaacactgccttcctggagactgacagcgtccagagacttggcagggcctcctgtctgccgcccttctcacatactggggcagtcggtgggctgagagagctctttggataccttggaaatggccggaaacctccagaaaatctcgcagaccagtcgtttctagaaaggctgctcctccctaaagctgacctggttggatcaaatgcaggggtgtctcgggctcaggtccctggccaccaacctgaggccaccaacctcatctccgtggcagtgaaagtgttgcaccctggcctgctcgctcaggtgcatatggacctgctgctgatgaagattggcagccgagtcctgggagttttgccaggcatcaagtggcttagcttgcctgagattgtggaggaatttgagaagctgatggtccaacagattgacctgcgttacgaagctcagaatctagaacacttccaggtcaacttccggaatgtgaaagccgtcaagttccccacccctctgcgcccctttgtcaccagagaagtcttggtggaaacgtatgaagagagtgtgcctgtgtccagttaccagcaggcaggaattcccgtggacttgaaaaggaagattgcacggctggggatcaacatgctcctgaagatgatatttgtggataactttgtccatgcagaccttcaccctggaaacatcctggttcagggtgccaacggcctgtcctcgagtcaggaggcgcagctgcagcaggcggacatctgtgacactctggtggtggccgtgccatcttccctctgcccgctgcgactggtgctgctggatgctggcattgtggcggagctgcaggcccctgacctgaggaatttccgggcagttttcatggctgtggtgatggggcagggccagagagtggctgagctgatcctgcatcatgcccgggccagcgagtgcagggacgtggaggggttcaaaaccgagatggccatgctggtgacccaggccaggaagaacaccatcaccctggagaagcttcatgtgtccagccttctctctagtgtctttaagttgctgatgactcacaaggtaaagcttgagagcaactttgcctccattgtgtttgccatcatggtgttggaggggcttggccgctcactggaccccaaactggacatcctggaggcagcgaggcccttcctcctcacgggcccagtgtgccccccgtga

SEQ ID NO: 29 - DNA, 1560 bp, Homo sapiens
atggatctggtaggagtggcatcgcctgagcccgggacggcagcggcctggggacccagcaagtgtccatgggctattcctcaaaatacaatatcttgttctttggctgatgtaatgagtgaacagctggccaaagaattgcagttagaagaagaagctgccgtttttcctgaagttgctgttgctgaaggaccatttattactggagaaaacattgatacttccagtgaccttatgctggctcagatgctacagatggaatatgacagagaatatgatgcacagcttaggcgtgaagaaaaaaaattcaatggagatagcaaagtttccatttcctttgaaaattatcgaaaagtgcatccttatgaagacagcgatagctctgaagatgaggttgactggcaggatactcgtgatgatccctacagaccagcaaaaccggttcccactcctaaaaagggctttattggaaaaggaaaagatatcaccaccaaacatgatgaagtagtatgtgggagaaagaacacagcaagaatggaaaattttgcacctgagtttcaggtaggagatggaattggaatggatttaaaactatcaaaccatgttttcaatgctttaaaacaacatgcctactcagaagaacgtcgaagtgcccgcctacatgagaaaaaggagcattctacagcagaaaaagcagttgatcctaagacacgtttacttatgtataaaatggtcaactctggaatgttggagacaatcactggctgtattagtacaggaaaggagtctgttgtctttcatgcatatggagggagcatggaggatgaaaaggaagatagtaaagttatacctacagaatgtgccatcaaggtatttaaaacaacccttaatgaatttaagaatcgtgacaaatatattaaagatgatttcaggtttaaagatcgcttcagtaaactaaatccacgtaagatccaccgcatgtgggcagaaaaagaaatgcacaatctcgcaagaatgcagagagctggaattccttgtccaacagttgtactactgaagaaacacattttagttatgtcttttattggccatgatcaagttccagcccctaaattaaaagaagtaaagctcaatagtgaagaaatgaaagaagcctactatcaaactcttcatttgatgcggcagttatatcatgaatgtacgcttgtccatgctgacctcagtgagtataacatgctgtggcatgctggaaaggtctggttgatcgatgtcagtcagtcagtagaacctacccaccctcacggcctggagttcttgttccgggactgcaggaatgtctcgcagtttttccagaaaggaggagtcaaggaagcccttagtgaacgagaactcttcaatgctgtttcaggcttaaacatcacagcagataatgaagctgattttttagctgagatagaagctttggagaaaatgaatgaagatcacgttcagaagaatggaaggaaagctgcttcatttttgaaagatgatggagacccaccactactatatgatgaatag

SEQ ID NO: 30 - DNA, 2397 bp, Homo sapiens
atggcggcttcgctcccgggacctgggagccggcttttccgcacatatggggctgcggacggcaggagacagcggcggccgggccgggaagccgcgcagtggttcccgccgcaggaccggaggcgtttcttcaacagcagcggcagcagcgacgccagcatcggcgacccctcgcagtccgacgatcctgacgatcccgacgaccccgacttccccggcagcccggtgaggcggcggcggaggtgtcccggcggccgagtgcccaaggaccggcccagcctgaccgtgaccccaaagcgctggaagctgcgagctcgcccaagcctaaccgtgaccccaagacgcctggggctgcgagctcggcccccgcagaagtgcagcacaccctgcggcccgctccgacttccgcccttccccagccgcgactccggccgcctcagcccggacctcagcgtgtgcggccagcccagggacggcgacgagctgggcatcagtgcctccctgttcagctctctggcctcgccctgccccgggtccccaacgccaagggacagtgtcatctcgatcggcacctccgcctgtctggttgcagcctcagccgtcccgagcgacctccacctcccagaagtctccctggaccgagcatctctcccctgctcccaggaggaagcgacaggaggagccaaggacaccaggatggtccaccaaacccgcgccagcctcaggtcagttctctttggccttatgaactcaggaacccctgaggattctgagtttcgggcagatgggaagaatatgagagagtcctgctgtaaaaggaaactggtggtgggaaatggaccagagggtccaggtctgtcaagcacaggcaagaggagggccacaggccaggactcttgtcaagagagagggcttcaagaggccgtccggagagagcatcaggaggccagtgttcccaagggccgcattgtgccaaggggaacagacaggctggagagaactagatcaagccggaagagcaaacatcaggaggcaacggaaacctctctcctccattcccaccgctttaaaaagggccaaaagctgggaaaagattcgttccccacccaggacctgactcctttacagaatgcctgcttttggaccaaaaccagggcttccttcagtttccacaagaagaaaattgtgactgatgtgtcagaggtctgcagcatctataccactgccacttctctctctggatccctcctatcagaatgttcaaaccggcctgtcatgaacagaacaagtggtgctccgtcctcttggcactcctcctctatgtatttgctaagccccttaaacactctaagtatttcaaacaaaaaggcatctgatgctgaaaaggtttatggggaatgcagtcagaagggtcctgtcccctttagccattgccttcccacagaaaaactgcaacgctgtgagaagattggggaaggggtgtttggcgaagtgtttcaaacaattgctgatcacacacccgtagccataaaaatcattgctattgaaggaccagatttagtcaatggatcccatcagaaaacctttgaggaaatcctgccagagatcatcatctccaaagagttgagcctcttatccggtgaagtgtgcaaccgcacagaaggctttatcgggctgaactcagtgcactgtgtccagggatcttaccctcccttgctcctcaaagcctgggatcactataattcaaccaaaggctctgcaaatgaccggcctgatttttttaaagacgaccagctcttcattgtgctggaatttgagtttggagggattgacttagagcaaatgcgaaccaagttgtcttccttggctactgcaaagagcattctacaccagctcacagcctccctcgcagtggcagaggcatcactgcgctttgagcaccgagacttacactgggggaacgtgctcttaaagaaaaccagcctcaaaaaactccactacaccctcaatgggaagagcagcactatccccagctgtgggttgcaagtgagcatcattgactacaccctgtcgcgcttggaacgggatgggattgtggttttctgtgacgtttccatggatgaggacctgtttaccggtgacggtgactaccagtttgacatctacaggctcatgaagaaggagaataacaaccgctggggtgaatatcacccttatagtaatgtgctctggttacattacctgacagacaagatgctgaaacaaatgaccttcaagactaaatgtaacactcctgccatgaagcaaattaagagaaaaatccaggagttccacaggacaatgctgaacttcagctctgccactgacttgctctgccagcacagtctgtttaagtaa

SEQ ID NO: 31 - DNA, 4542 bp, Homo sapiens
atgtcccagactgaggccgacctggccctgcggcccccgcctcctcttggcaccgcggggcagccccgcctcgggccccctcctcgccgagcgcgccgcttctccgggaaggctgagccccggccgcgctcttctcgtctcagccgccgtagctcagtcgacttggggctgctgagctcttggtccctgccagcctcacccgctccggacccccccgatcctccggactccgctggtcctggccccgcgaggagcccaccgcctagctccaaagaaccccccgagggcacgtggaccgagggagcccctgtgaaggctgcggaagactccgcgcgtcccgagctcccggactctgcagtgggcccggggtccagggagccgctaagggtccctgaagctgtggccctagagcggcggcgggagcaggaagaaaaggaggacatggagacccaggctgtggcaacgtcccccgatggccgatacctcaagtttgacatcgagattggacgtggctccttcaagacggtgtatcgagggctagacaccgacaccacagtggaggtggcctggtgtgagctgcagactcggaaactgtctagagctgagcggcagcgcttctcagaggaggtggagatgctcaaggggctgcagcaccccaacatcgtccgcttctatgattcgtggaagtcggtgctgaggggccaggtttgcatcgtgctggtcaccgaactcatgacctcgggcacgctcaagacgtacctgaggcggttccgggagatgaagccgcgggtccttcagcgctggagccgccaaatcctgcggggacttcatttcctacactcccgggttcctcccatcctgcaccgggatctcaagtgcgacaatgtctttatcacgggacctactggctctgtcaaaatcggggacctgggcctggccacgctcaagcgcgcctcctttgccaagagtgtcatcgggaccccggaattcatggcccccgagatgtacgaggaaaagtacgatgaggccgtggacgtgtacgcgttcggcatgtgcatgctggagatggccacctctgagtacccgtactccgagtgccagaatgccgcgcaaatctaccgcaaggtcacttcgggcagaaagccgaacagcttccacaaggtgaagatacccgaggtgaaggagatcattgaaggctgcatccgcacggataagaacgagaggttcaccatccaggacctcctggcccacgccttcttccgcgaggagcgcggtgtgcacgtggaactagcggaggaggacgacggcgagaagccgggcctcaagctctggctgcgcatggaggacgcgcggcgcggggggcgcccacgggacaaccaggccatcgagttcctgttccagctgggccgggacgcggccgaggaggtggcacaggagatggtggctctgggcttggtctgtgaagccgattaccagccagtggcccgtgcagtacgtgaacgggttgctgccatccagcgaaagcgtgagaagctgcgtaaagcaagggaattggaggcactcccaccagagccaggacctccaccagcaactgtgcccatggcccccggtccccccagtgtcttcccccctgagcctgaggagccagaggcagaccagcaccagcccttccttttccgccacgccagctactcatctaccacttcggattgcgagactgatggctacctcagctcctccggcttcctggatgcctcagaccctgcccttcagccccctgggggggtgccatccagcctggctgagtcccatctctgcctgccctcggcttttgccctatccattccacgttctggccctggaagtgacttttcccccggggacagctatgcctcagatgcagcttcaggccttagcgatgtgggagaagggatgggacaaatgaggagacccccagggaggaatctccggcgcagaccccgatcccggctgcgggtcactagtgtctcagaccagaatgacagagtggttgagtgccagctacagacccataacagcaagatggtgaccttccgatttgatctggatggggacagcccggaagagattgcagctgccatggtatataacgagttcattctgccttcggagcgagatggatttctcagacggattcgggagattatccagcgagtggagaccctgttgaagagagacactggccccatggaggctgctgaagacaccctaagcccccaggaggagccagcaccattacctgccctgcccgtccccctcccagacccatccaatgaagagctccagagcagcacctccctggagcacaggagctggacagccttctccacctcctcatcttctcctggaactcctttgtctcctggaaacccattttcccctggaacccccatttccccaggtcccatcttccccatcacttctcccccatgtcatcccagcccctccccattctcccccatttcttcccaggtctcctcaaatccctctccacaccccaccagctctccacttccattctcctccagcacacccgagtttccggtcccactctctcagtgtccctggagttctctccccacgacttctccacctacgttctctcccacttgttctcaggtcactcttagttcccctttctttcctccgtgcccctccacttcttccttcccctccaccacagcagcccctctcctttctctggctagtgccttctcactggctgtgatgactgtggcccagtccctgctgtccccctcacctgggctcctttcccagtctcctccagcccctcctagtcccctccctagcctgccccttccccctcccgttgctcctggtggccaggaaagcccttcaccccacacagctgaggtggagagtgaggcctcaccacctcctgctcggcccctcccaggggaagccaggctggcgcccatctctgaagagggaaagccgcagcttgttgggcgtttccaagtgacttcatccaaggaaccggctgagcctcttcccttgcagccaacatcccccactctctctggttctccaaaaccttcaacccctcagctcacttcagagagctcagatacagaggacagtgctggaggcgggccagagaccagggaagctctggctgagagcgaccgtgcagctgagggtctgggggctggagttgaggaggaaggagatgatgggaaggaaccccaagttgggggcagcccccaacccctgagccatcccagcccagtgtggatgaactactcctacagcagcctgtgtttgagcagcgaggagtcagaaagcagtggggaagatgaggagttctgggctgagctgcagagtcttcggcagaagcacttgtcagaggtggaaacactacagacactacagaaaaaagaaattgaagatttgtacagccggctggggaagcagcccccaccgggtattgtggccccagctgctatgctgtccagccgccagcgccgcctctccaagggcagcttccccacctcccgccgcaacagcctacagcgctctgagcccccaggccctggcatcatgcgaaggaactctctgagtggcagcagcaccggctcccaggagcagcgggcaagcaagggggtgacattcgccggggatgttggcaggatgtttggagttgtcgccacagagacgattgaagacgccctgcttcacttggcccagcagaatgagcaagcagtgagggaggcttcggggcggctgggccgcttcagggagccccagatcgtggagtttgtttttctcctgtctgaacaatggtgtctggagaaatctgtgagctaccaggctgtagaaatcctagaaaggtttatggtaaaacaggcagagaacatctgcaggcaagccacaatccagccaagagataataagagagagtctcagaattggagggctctgaaacagcagcttgtcaacaagtttactctccgtcttgtgtcatgtgttcagctggccagcaaactttccttccgaaacaaaataatcagcaacattccagtcttgaatttcctccaggctctaggctatctacacactaaagaagaactgctggaatcagagcttgatgttttgaagtccttgaacttccgaattaatctgcccactcccctggcatatgtggagacgctcctagaggttttaggatacaatggctgtttggttccagccatgaggctgcatgcaacctgcctgacactgctcgacctggtctatcttctgcatgaacccatatatgagagcctgttgagggcttcaattgagaactccactcccagtcagctgcaaggggaaaagtttacttcagtgaaggaagacttcatgctgttggcagtaggaatcattgcagcaagtgctttcatccaaaaccatgagtgttggagccaggtatgcaccactgagcaggaccagcatgagagagttaaggtggacgagcatgccctttttgtcatatcagcctga

SEQ ID NO: 32 - DNA, 1065 bp, Homo sapiens
tcggagactgactgctacgacatcatcgaggtcttgggcaaggggaccttcggggaggtagccaagggctggcggcggagcacgggcgagatggtggccatcaagatcctcaagaatgacgcctaccgcaaccgcatcatcaagaacgagctgaagctgctgcactgcatgcgaggcctagaccctgaagaggcccacgtcatccgcttccttgagttcttccatgacgccctcaagttctacctggtctttgagctgctggagcaaaaccttttcgagttccagaaggagaacaacttcgcgcccctccccgcccgccacatccgtacagtcaccctgcaggtgctcacagccctggcccggctcaaggagctggctatcatccacgctgatctcaagcctgagaacatcatgctggtggaccagacccgctgccccttcagggtcaaggtgattgacttcggatccgccagcattttcagcgaggtgcgctacgtgaaggagccatacatccagtcgcgcttctaccgggcccctgagatcctgctggggctgcccttctgcgagaaggtggacgtgtggtccctgggctgcgtcatggctgagctgcacctgggctggcctctctaccccggcaacaacgagtacgaccaggtgcgctacatctgcgaaacccagggcctgcccaagccacacctgttgcacgccgcctgcaaggcccaccacttcttcaagcgcaacccccaccctgacgctgccaacccctggcagctcaagtcctcggctgactacctggccgagacgaaggtgcgcccattggagcgccgcaagtatatgctcaagtcgttggaccagattgagacagtgaatggtggcagtgtggccagtcggctaaccttccctgaccgggaggcgctggcggagcacgccgacctcaagagcatggtggagctgatcaagcgcatgctgacctgggagtcacacgaacgcatcagccccagtgctgccctgcgccaccccttcgtgtccatgcagcagctgcgcagtgcccacgagaccacccac

SEQ ID NO: 33 - DNA, 2112 bp, Homo sapiens
atgaacacccctctgcagtctgcacatacacaccttaccctggtcgagttcatagcctacttggtgtctgactccccctttcctgctgcttcaaccttgtctctgcccacgtgcaactttgatccctgccccactaaactgggtgctggcacaatgcctcccgcagcggcgggcggtcagcgccggcctcatgcagctcctcccgccgcctatccgcaccggaacaaagtaagggccgcggaggctcgtacttcacgagcagtcaggcgcgagtacgaggcaggcgacgaagcttctggtcgacggggtcgtggggggagaatgaaaagcgtgcagaatgcaagagctgaccggggaatgtgggtcagagagaggcgggctgtggtgcgcggacaagaaaatacagttgtagtttttgcagcagctgcatccccggaatccgacttcctccaaatcgtgcttcgtgatattgccagagaaatggtaccagaatacaaatctgatcttcagcccaggactcaaatggatgctaaaaagccaaggaaatgtgatttgactcccttcctggttttgaaagcaagaaagaaacaaaagttcacctctgcgaagcacctctgggtacagcgcaaaggcaaaggtgtggaggatcatgctgattggcagggccaaatctgggtgggtgtggagggaaaatcagcagaagggcaaggccttaatctaacaggtgcatttaacagaagtccagaaaacaaagtatctccccaaggccctgcactgaatgcattagaaattgactccagctatctggacccccaggctcttgcccttcctcaaggatggcagggcctcctctttccacctctactagggacagagctggttggaagtaaactttcggttcagatccagaagccaccttccaatatcaagaactccagaatgacccaagtctttcataagaacaccagtgttacttcactcccctttgtggacaccaaggggaagaagaatacggtaagcttcccacacattagcaagaaagtcctgctgaagtcatccctgctgtatcaggtgagtgcagacggactggaccctgagaaggcaggtgaaagacctggttttagtccaaagccagttttagaaaggcccaggatagtggggaaaagcacagtagctgctgaagaggagaatcaagctcacaatcagatgccggcctcagagctcaaggcttcagaaatacctttccaccctagcattaaaacccaggatcccaaggcagaggagaagtcaccaaagaagcaaaaggtgactctgacagcggcagaggccctaaagctttttaagaaccagctgtctccatatgaacaaagtgaaatcctgggctacgcggagctgtggttcctgggtcttgaagccaagaagctcgacacggctcctgagaaatttagcaagacgagttttgatgatgagcatggcttctatctgaaggtcctgcatgatcacattgcctaccgctatgaagttctggagacaatcgggaaggggtcctttggacaggtggccaagtgcttggatcacaaaaacaatgagctggtggccctgaaaatcatcaggaacaagaagaggtttcaccagcaggccctgatggagctgaagatcctggaagctctcagaaagaaggacaaagacaacacctacaatgtggtgcatatgaaggactttttctactttcgcaatcacttctgcatcacctttgagctcctgggaatcaacttgtatgagttgatgaagaataacaactttcaaggcttcagtctgtccatagttcggcgcttcactctctctgttttgaagtgcttgcagatgctttcggtagagaaaatcattcactgtgatctcaagcccgaaaatatagtgctataccaaaagggccaagcctctgttaaagtcattgactttggatcaagctgttatgaacaccagaaaggtcacctgccttctaggctgcgctcagctgtcaccactaccagtatgattttgaacaaggtgcttcgcttctctgagcccaagttgcctcatctacaatttgagaaagttgtatccaaatactag

SEQ ID NO: 34 - DNA, 1260 bp, Homo sapiens
atggcgggatccggctgcgcctggggcgcggagccgccgcgttttctggaggccttcgggcggctgtggcaggtacagagccgtctgggtagcggctcctccgcctcggtgtatcgggttcgctgctgcggcaaccctggctcgccccccggcgccctcaagcagttcttgccgccaggaaccaccggggctgcggcctctgccgccgagtatggtttccgcaaagagagggcggcgctggaacagttgcagggtcacagaaacatcgtgactttgtatggagtgtttacaatccacttttctccaaatgtgccatcacgctgtctgttgcttgaactcctggatgtcagtgtttcggaattgctcttatattccagtcaccagggttgttccatgtggatgatacagcattgtgcccgagatgttttggaggcccttgcttttcttcatcatgagggctatgtccatgcggacctcaaaccacgtaacatattgtggagtgcagagaatgaatgttttaaactcattgactttggacttagcttcaaagaaggcaatcaggatgtaaagtatattcagacagacgggtatcgggctccagaagcagaattgcaaaattgcttggcccaggctggcctgcagagtgatacagaatgtacctcagctgttgatctgtggagcctaggaatcattttactggaaatgttctcaggaatgaaactgaaacatacagtcagatctcaggaatggaaggcaaacagttctgctattattgatcacatatttgccagtaaagcagtggtgaatgccgcaattccagcctatcacctaagagaccttatcaaaagcatgcttcatgatgatccaagcagaagaattcctgctgaaatggcattgtgcagcccattctttagcattccttttgcccctcatattgaagatctggtcatgcttcccactccagtgctaagactgctgaatgtgctggatgatgattatcttgagaatgaagaggaatatgaagatgttgtagaagatgtaaaagaggagtgtcaaaaatatggaccagtggtatctctacttgttccaaagggaaatcctggcagaggacaagtctttgttgagtatgcaaatgctggtgattccaaagctgcgcagaaattactgactggaaggatgtttgatgggaagtttgttgtggctacattctacccgctgagtgcctacaagaggggatatctgtatcaaaccttgctttaa

SEQ ID NO: 35 - DNA, 1986 bp, Homo sapiens
atgaagaagttctctcggatgcccaagtcggagggcggcagcggcggcggagcggcgggtggcggggctggcggggccggggccggggccggctgcggctccggcggctcgtccgtgggggtccgggtgttcgcggtcggccgccaccaggtcaccctggaagagtcgctggccgaaggtggattctccacagttttcctcgtgcgtactcacggtggaatccgatgtgcattgaagcgaatgtatgtcaataacatgccagacctcaatgtttgtaaaagggaaattacaattatgaaagagctatctggtcacaaaaatattgtgggctatttggactgtgctgttaattcaattagtgataatgtatgggaagtccttatcttaatggaatattgtcgagctggacaggtagtgaatcaaatgaataagaagctacagacgggttttacagaaccagaagtgttacagatattctgtgatacctgtgaagctgttgcaaggttgcatcagtgtaagactccaataattcaccgggatctgaaggtagaaaatattttgttgaatgatggtgggaactatgtactttgtgactttggcagtgccactaataaatttcttaatcctcaaaaagatggagttaatgtagtagaagaagaaattaaaaagtatacaactctgtcatacagagcccctgaaatgatcaacctttatggagggaaacccatcaccaccaaggctgatatctgggcactgggatgtctactctataaactttgtttcttcactcttccttttggtgagagtcaggttgctatctgtgatggcaacttcaccatcccagacaattctcgttactcccgtaacatacattgcttaataaggttcatgcttgaaccagatccggaacatagacctgatatatttcaagtgtcatattttgcatttaaatttgccaaaaaggattgtccagtctccaacatcaataagtgttgtaaacaattactgagacacggagcactgttaactgaaattcttctattccttcagctcttcctgaaccgaatgactgctagtgaagcagctgctaggaaaagccaaataaaagccagaataacagataccattggaccaacagaaacctcaattgcaccaagacaaagaccaaaggccaactctgctactactgccactcccagtgtgctgaccattcaaagttcagcaacacctgttaaagtccttgctcctggtgaattcggtaaccatagaccaaaaggggcactaagacctggaaatggccctgaaattttattgggtcagggacctcctcagcagccgccacagcagcatagagtactccagcaactacagcagggagattggagattacagcaactccatttacagcatcgtcatcctcaccagcagcagcagcagcagcagcagcagcagcagcaccaccaccaccaccaccaccacctacttcaagatgcttatatgcagcagtatcaacatgcaacacagcagcaacagatgcttcaacaacaatttttaatgcattcggtatatcaaccacaaccttctgcatcacagtatcctacaatgatgccgcagtatcagcaggctttctttcaacagcagatgctagctcaacatcagccgtctcaacaacaggcatcacctgaatatcttacctcccctcaagagttctcaccagccttagtttcctacacttcatcacttccagctcaggttggaaccataatggactcctcctatagtgccaataggtcagttgctgataaagaggccattgcaaatttcacaaatcagaagaacatcagcaatccacctgatatgtcagggtggaatccttttggagaggataatttctctaagttaacagaagaggaactattggacagagaatttgaccttctaagatcaagtaagggacacttgaaggcttattttgcttcacagtaa

SEQ ID NO: 36 - DNA, 4848 bp, Homo sapiens
atgtttccattaattggaaaaacaatcatctttgataactttcctgatccttctgatacatgggaaatcactgagacaattggcaaaggaacttatgggaaagtttttaaagtattgaataagaaaaatggccaaaaagcagcagtcaaaattcttgatccaattcacgatattgacgaagagattgaagcagaatataacatcttaaaagcactttctgaccaccctaatgtggtcagattctatgggatatactttaagaaggataaagtaaatggagacaagctgtggttggttcttgagctctgcagtggaggatcagtgactgaccttgtgaaaggatttctgaagaggggtgaaagaatgagtgagcctctaattgcctatattttacatgaagcactaatgggacttcaacatttgcataacaacaaaactatccacagagatgtgaaaggcaataacattctattgaccacggaaggtggagtgaaactagtagattttggtgtgtctgcacagctcaccagtacccggcaccgtcggaacacatccgtaggaacaccgttttggatggctcctgaggtgattgcatgtgaacagcaattggataccacttatgacgccagatgtgacacttggtccctgggtatcacggccattgagctgggtgatggagatcctccactagctgaccttcatcccatgagagcactcttcaaaataccaaggaatccacccccaaaactaaggcagcctgagctatggtcagcagaattcaatgacttcataagcaagtgcttgactaaagattatgaaaagcgtccaacagtgtcagaacttttacagcataaattcattactcaaattgagggcaaagatgtgatgctacaaaaacaactaacggaattcattggcatccatcaatgcatgggaggcacagaaaaggccagacgtgaacgtattcacacgaagaaagggaacttcaaccgacctctaatatccaatctgaaggatgtagatgatttagcaaccctagaaattttggatgagaatacagtctcagagcaacttgagaagtgttattccagagatcagatctacgtctatgtgggagacatactcattgctcttaacccttttcagagtctgggtctttactccacaaagcattccaaactatatattggatcaaagagaactgccagtcctcctcacatttttgcaatggctgacttaggatatcaatctataataacatataattcagatcagtgcattgttatttctggagaaagtggtgctggaaagactgaaaatgctcatcttttagttcagcagctgacagtgcttggaaaggctaataacagaaccttgcaagagaagattttacaagtgaacaatttggtagaagcctttggcaatgcctgcactattataaatgacaattctagcagatttggaaaatacttagaaatgaaattcacctcttctggagcggtagtgggagcacagatttctgaatatctcctggaaaaatcccgagttatccaccaagctattggagaaaaaaattttcatattttttactacatttatgctggtttggctgaaaagaagaaactagcccattacaaactgcctgaaaataagcctcccaggtacctacaaaatgaccacctcagaacagtacaagacatcatgaataatagtttctataaatcccagtatgaattaattgagcaatgtttcaaagtcataggttttacaatggagcaacttggtagtatatacagcatactcgctgcaatcttgaatgttggcaacattgaattttcttctgtggcaactgaacaccagattgacaagagccacatttctaatcatacagccctggagaactgtgtttctttgctttgcattcgggcagatgagctacaagaagctctcacctcccactgtgtggtcactagaggagaaacaattatacgacccaatactgtagaaaaagctaccgatgtcagggatgccatggctaaaactttatatggacgtctctttagttggatagtcaattgcattaacagtttgttgaagcatgactcatcaccaagtgggaatggtgatgagctgagcattggcattcttgatatatttggttttgaaaatttcaaaaaaaattccttcgagcagctgtgcattaacattgcaaatgaacaaattcagtattattataatcaacatgtgtttgcatgggaacagaatgaatacctaaatgaagatgtggatgctagagttattgaatatgaggataactggcccctcttagatatgtttctgcaaaagccaatgggtttactttccctacttgatgaagaaagtagatttcccaaggccactgaccagactcttgtagaaaaatttgaaggtaacctgaaatcacaatacttctggagacccaaaagaatggaacttagttttggaattcaccattatgcaggaaaggtcctctataatgcaagtggattcttagccaaaaacaagacactagttcctactgacattgtgctacttttgaggtcatccgacaacagtgtaattaggcaactagtcaaccaccctctgaccaaaacaggtaatctgccacattctaaactaaaaatgttaaactatcaaatgaggacttcagaaaaattaatcaacctggcaaagggcgacactggagaagccacacgtcatgccagagagacaaccaacatgaaaacacaaacggttgcatcatattttagatattccctgatggatttgttgtctaaaatggtggtgggccaacctcattttgtccgttgcatcaaaccaaatagtgagcgtcaggcaagaaaatatgacaaagagaaagttctgctacagcttcggtacacaggaattctggaaacagcaagaattcgaagactaggattctcccatcggatactttttgctaactttataaagcggtactaccttctctgctacaagtcgagcgaggagccccgcatgagccctgacacctgtgccaccattttggaaaaagctggtctcgataactgggctcttggaaaaacaaaagtgttccttaagtattatcacgtggagcagttaaatctaatgcgaaaggaagctattgacaagcttattttgattcaagcttgtgtcagagcattcttgtgttcaagaagataccaaaaaatacaggagaaaaggaaagaaagcgctataataatacagtcagctgcaagaggacaccttgtcgggaaacaaagaaaagaaattgttgacatgaaaaacacagcagtaacaaccattcaaacttctgatcaggaattcgactacaagaaaaactttgaaaatacaagggaatctttcgtgaagaaacaagcagaaaatgcaatctctgctaatgaaagattcatttcagctccaaataataaaggaagtgtatctgtagtgaagacttccactttcaaacctgaagaggaaaccaccaatgctgtggagagtaacaacagagtgtatcagactccaaaaaaaatgaataatgtgtatgaggaagaggttaagcaagaattttaccttgtagggccagaagtaagccccaaacagaagtttgtcaaagacctggaagagaacagcaatctaaggaaagtggagaaagaggaagctatgatccagagttactatcagaggtacacagaggagaggaattgtgaagagtcaaaagcagcatatctagaaaggaaggccatatcagaaaggccaagctacccagtgccttggttagctgaaaatgagacttcctttaaaaaaactttggaacctacacttagccaaaggtcaatttatcaaaatgcaaacagcatggaaaaagaaaagaagacatctgtagttacccagagtgcaccgatatgcagccaggaggaaggcagaggccgtctgaggcatgagacagtcaaagagaggcaagttgaaccagtgacacaggcccaggaggaagaagataaagcagcggtattcattcagagcaaataccggggttacaagagaaggcagcagttgaggaaggacaagatgtcttcttttaagcatcagaagattgtcacaacaccaacagaagtagcaagaaacactcataatttgtattcctatcccacaaaacatgaggaaatcaataacatcaagaagaaggataacaaagactcgaaagcaacttcagaaagagaagcatgtggtttggcaattttttcaaaacagatatcaaagttatctgaagaatatttcattctgcagaaaaaattgaatgaaatgattttgtcacagcaactgaagtcactttatctgggtgtctcgcaccataagccaattaatagacgagtttcttctcagcagtgcctctcaggtgtctgtaaaggagaggagccaaaaatattgagacccccaagacgaccccggaaacccaaaacattaaataaccctgaagactccacatactattatctacttcataagtcaatccaagaagaaaaacgaagaccaaggaaagacagtcagggaaaattattagatttggaagatttctattataaggaatttttgcccagtcgttctggaccaaaggaacatagccctagtttaagagaacgaagaccacagcaagaactccagaatcaatgtattaaggctaatgaaaggtgctgggcggcggagagccccgagaaggaggaggagagagagccagcagccaacccctacgacttcaggaggctcctgcgcaaaacctcccagcgccggcgcctcgtccagcagtcctaa

SEQ ID NO: 37 - DNA, 204 bp, Homo sapiens
tttaaagttggaaatctgcctggtaagggatcatttgctggtgtctgcaaagttgagtccatacacactggtttggaaatttcagtccagatgatagttaagaaagcagtaagaatacagagagtccacaatgagatgaaaatgcactgccagttgaaacatcctcctacactggagctttataaatattttaaagacaaggat

SEQ ID NO: 38 - DNA, 2838 bp, Homo sapiens
atgacgaaaagcgaggagcagcagcctctgagtttgcaaaaagccttacagcagtgcgaactggtccaaaacatgatagacttgagcatctccaacctggaagggcttaggaccaaatgtgctacctccaacgacctcacacaaaaagaaatccggaccctggagagcaagctggtgaagtacttcagccggcagctgtcctgcaaaaagaaggtagccttgcaggagcgcaacgcggagctggacggcttcccccagctacggcactggttccgaatcgtcgatgtgcgcaaggaggtcctggaggaaatctcccccggccagctgagcctggaggacctcttggagatgacggatgaacaggtgtgcgagactgtggagaaatacggagccaaccgggaggagtgtgcccgcctcaacgcctccctctcctgcctcaggaatgtccacatgtcaggaggcaacctttccaaacaagactggaccatccagtggcccaccacagagacggggaaggagaacaatcccgtgtgccccccggagcccaccccgtggatccgcacccatctctcccagagccccagggtcccgtccaagtgcgtccagcactattgtcacaccagccccactcccggggcccctgtgtacacccacgtggacaggcttaccgtggacgcctacccgggcttgtgcccgcccccgccactggagtcgggccaccgttccctgcccccatcgccccggcagcggcacgcggtccgcaccccgccgcgcacccccaacatcgtcaccaccgtgaccccgccgggcacgccgcccatgaggaagaagaacaagctgaagcccccggggaccccaccgccctcctcccgaaaactgatacacttgatcccgggattcaccgcgctgcatcggagcaaatcccacgagttccagctggggcaccgcgtggacgaggcccacacgcccaaagccaagaagaagagcaaacccttgaacctcaagatccacagcagcgtaggcagctgcgagaacatcccctctcagcagcgctccccgctgctgtccgagcgctccctccgctccttctttgtgggacacgcacctttcctgccttccacccctcctgttcacactgaggccaacttctctgcaaacacactgtcagtgccacgctggtccccgcagatccctcgcagagatcttggcaactccatcaagcacaggttttccaccaagtactggatgtctcagacgtgcacagtctgtgggaaagggatgctttttggcctcaagtgtaaaaactgcaagttaaagtgccacaacaaatgcaccaaagaagccccaccctgtcatcttctgatcatccaccgaggagatccagcaaggttagtccggacagagtccgttccgtgtgacatcaacaaccctctacggaagccacctcgctattcagacctgcacatcagtcagacgctccccaaaaccaacaaaatcaacaaggaccacatccctgtcccttaccagccagactccagcagcaacccctcctccacgacgtcctccacgccctcctcgccagcaccccccctccctcctagtgccacgccgccttctcccctacacccttccccacagtgcacacggcagcagaagaacttcaacctgccagcatcccactactacaaatacaagcagcagttcatcttcccagatgtggtgccggtgccggagacgccgacccgggcgccccaggtcatcctgcatccggtgacctcgaatccaatcttggaaggaaatccattacttcaaattgaagtggagccaacgtcggagaatgaagaggtccatgatgaggccgaagagtcagaggatgacttcgaggagatgaacctgtccctcctctcggcccggagcttcccacgcaaggccagccagaccagcatcttccttcaggagtgggacatcccctttgagcagctggagatcggcgagctcattggaaagggccgctttgggcaagtgtaccacggccgctggcatggcgaggtggccatccggctgattgacattgagagggacaacgaggaccagctcaaggccttcaagcgggaggtgatggcctacaggcagacacggcatgagaacgtggtgcttttcatgggtgcctgcatgagcccgcctcacctggccatcatcaccagcctctgtaagggacggacgctctattccgttgtgagggatgccaaaatcgttttggatgtcaacaaaaccaggcagattgctcaagaaattgtgaagggcatgggctacctccacgccaagggaatcctacacaaggacctcaagtcaaagaacgtcttctatgacaacggcaaagtggtcatcacggactttggactcttcagcatttctggggtgctgcaggctggcaggcgggaggacaaactgcgcatccagaatggctggctatgccacctggcaccagagatcatccgccagctgtcccccgacacagaggaggataagctccccttctccaagcactctgacgtctttgcccttggcacaatctggtatgaactccacgccagggaatggcctttcaagacccaaccagcagaggcaataatctggcaaatgggcacaggcatgaaacccaacctcagccagattggcatgggaaaagaaatctcggacattcttctcttctgctgggcctttgaacaagaagagagacctaccttcaccaagctcatggacatgctggagaaactgccaaagcgaaaccgtcgcctgtctcaccctggacatttctggaagtctgcagagctgtga

SEQ ID NO: 39 - DNA, 2499 bp, Homo sapiens
atggagggcgacggcgggaccccatgggccctggcgctgctgcgcaccttcgacgcgggcgagttcacgggctgggagaaggtgggctcgggcggcttcgggcaggtgtacaaggtgcgccatgtccactggaagacctggctggccatcaagtgctcgcccagcctgcacgtcgacgacagggagcgcatggagcttttggaagaagccaagaagatggagatggccaagtttcgctacatcctgcctgtgtatggcatctgccgcgaacctgtcggcctggtcatggagtacatggagacgggctccctggaaaagctgctggcctcggagccattgccatgggatctccggttccgcatcatccacgagacggcggtgggcatgaacttcctgcactgcatggccccgccactcctgcacctggacctcaagcccgcgaacatcctgctggatgcccactaccacgtcaagatttctgattttggtctggccaagtgcaacgggctgtcccactcgcatgacctcagcatggatggcctgtttggcacaatcgcctacctccctccagagcgcatcagggagaagagccggctcttcgacaccaagcacgatgtatacagctttgcgatcgtcatctggggcgtgctcacacagaagaagccgtttgcagatgagaagaacatcctgcacatcatggtgaaggtggtgaagggccaccgccccgagctgccgcccgtgtgcagagcccggccgcgcgcctgcagccacctgatacgcctcatgcagcggtgctggcagggggatccgcgagttaggcccaccttccaaggaaacgggctgaatggggagctcatccgccaggtgctggcagctctgctccctgtgactggcaggtggcgctcccccggggaaggcttccgccttgagtctgaagtcatcatccgagtgacatgtcccctttcttccccacaagaaattacttctgaaaccgaggacctgtgtgaaaagcctgatgacgaagtgaaagaaactgctcatgatctggacgtgaaaagccccccggagcccaggagcgaggtggtgcctgcgaggctcaagcgggcctctgcccccaccttcgataacgactacagcctctccgagcttctctcacagctggactctggagtttcccaggctgtcgagggccccgaggagctcagccgcagctcctctgagtccaagctgccatcgtccggcagtgggaagaggctctcgggggtgtcctcggtggactccgccttctcttccagaggatcactgtcgctgtcctttgagcgggaaccttcaaccagcgatctgggcaccacagacgtccagaagaagaagcttgtggatgccatcgtgtccggggacaccagcaaactgatgaagatcctgcagccgcaggacgtggacctggcactggacagcggtgccagcctgctgcacctggcggtggaggccgggcaagaggagtgcgccaagtggctgctgctcaacaatgccaaccccaacctgagcaaccgtaggggctccaccccgttgcacatggccgtggagaggagggtgcggggtgtcgtggagctcctgctggcacggaagatcagtgtcaacgccaaggatgaggaccagtggacagccctccactttgcagcccagaacggggatgagtctagcacacggctgctgttggagaagaacgcctcggtcaacgaggtggactttgagggccggacgcccatgcacgtggcctgccagcacgggcaggagaatatcgtgcgcatcctgctgcgccgaggcgtggacgtgagcctgcagggcaaggatgcctggctgccactgcactacgctgcctggcagggccacctgcccatcgtcaagctgctggccaagcagccgggggtgagtgtgaacgcccagacgctggatgggaggacgccattgcacctggccgcacagcgcgggcactaccgcgtggcccgcatcctcatcgacctgtgctccgacgtcaacgtctgcagcctgctggcacagacacccctgcacgtggccgcggagacggggcacacgagcactgccaggctgctcctgcatcggggcgctggcaaggaggccgtgacctcagacggctacaccgctctgcacctggctgcccgcaacggacacctggccactgtcaagctgcttgtcgaggagaaggccgatgtgctggcccggggacccctgaaccagacggcgctgcacctggctgccgcccacgggcactcggaggtggtggaggagttggtcagcgccgatgtcattgacctgttcgacgagcaggggctcagcgcgctgcacctggccgcccagggccggcacgcacagacggtggagactctgctcaggcatggggcccacatcaacctgcagagcctcaagttccagggcggccatggccccgccgccacactcctgcggcgaagcaagacctag

SEQ ID NO: 40 - DNA, 1104 bp, Homo sapiens
atggatgacgctgctgtcctcaagcgacgaggctacctcctggggataaatttaggagagggctcctatgcaaaagtaaaatctgcttactctgagcgcctgaagttcaatgtggcgatcaagatcatcgaccgcaagaaggcccccgcagacttcttggagaaattccttccccgggaaattgagattctggccatgttaaaccactgctccatcattaagacctacgagatctttgagacatcacatggcaaggtctacatcgtcatggagctcgcagtccagggcgacctcctcgagttaatcaaaacccggggagccctgcatgaggacgaagctcgcaagaagttccaccagctttccttggccatcaagtactgccacgacctggacgtcgtccaccgggacctcaagtgtgacaaccttctccttgacaaggacttcaacatcaagctgtccgacttcagcttctccaagcgctgcctgcgggatgacagtggtcgaatggccttaagcaagaccttctgtgggtcaccagcgtatgcggccccagaggtgctgcagggcattccctaccagcccaaggtgtacgacatctggagcctaggcgtgatcctctacatcatggtctgcggctccatgccctacgacgactccaacatcaagaagatgctgcgtatccagaaggagcaccgcgtcaacttcccacgctccaagcacctgacaggcgagtgcaaggacctcatctaccacatgctgcagcccgacgtcaaccggcggctccacatcgacgagatcctcagccactgctggatgcagcccaaggcacggggatctccctctgtggccatcaacaaggagggggagagttcccggggaactgaacccttgtggacccccgaacctggctctgacaagaagtctgccaccaagctggagcctgagggagaggcacagccccaggcacagcctgagacaaaacccgaggggacagcaatgcaaatgtccaggcagtcggagatcctgggtttccccagcaagccgtcgactatggagacagaggaagggcccccccaacagcctccagagacgcgggcccagtga

SEQ ID NO: 41 - DNA, 93 bp, Homo sapiens
cccagggaggctgtctccattgtgtcctcctgctgggtgaggcccccggccaccagactccgagcacttaagattctgacccatcccgcattt

SEQ ID NO: 42 - DNA, 1053 bp, Homo sapiens
atggaaaagcagccccagaacagcaggagaggcctcgccccccgagaggtgccgccagctgttgggctgctgctgatcatggccctgatgaatactctactctacctctgcctcgaccacttcttcatcgctcctcgacaatccactgtggaccccacacactgtccctatggtcacttcaggataggacagatgaaaaactgctcaccttggctgtcctgcgaggagctgagaacagaagtgagacagctgaagcgtgttggggaaggagctgtaaagagagtctttctgtctgagtggaaggagcacaaagttgcactctcacagctcaccagcctggagatgaaagatgatttcctccatggactgcagatgctgaaatctctccaaggcacacatgttgtcacgctgcttggctattgtgaggatgacaacactatgcttactgaatatcaccctctaggttccttgagtaacctggaagaaacactaaacctttcaaagtaccaaaatgtgaacacgtggcagcacaggctggagctggccatggactatgtcagcatcattaattacctgcaccacagccctgtgggcacacgggtcatgtgcgactccaacgacctgccgaagacactgtcccagtatctgctaacaagcaacttcagcattttggcaaatgacttggacgccttacccctggtgaaccacagctccgggatgctggtgaagtgcggccacagggagctgcatggggatttcgtggctccagagcaactgtggccctatggagaggacgtgcctttccacgatgatctcatgccctcatatgatgagaagattgacatttggaagatcccagacatctccagtttccttctggggcacattgaagggagtgatatggtccgattccatttgtttgatattcacaaagcatgcaagagccagactccctcagaaagacccactgcccaggacgttctggagacctaccagaaggtcttggatacacttagagatgccatgatgtctcaggcaagagagatgctgtga

SEQ ID NO: 43 - DNA, 1419 bp, Homo sapiens
atggataatattgatgaaatcctagagaagactgagtcaagtgtctgcaaagagctggagatagctctggttgatcaaggtgatgcagacaaggagataatttcaaatacatatagtcaagtactgcaaaagattcattcagaggaaaggctcattgccacagtacaagctaagtacaaggacagtattgagtttaaaaagcagcttattgaatatttaaagaagattcccagtgtggatcacttgctatccattaagaagacattgaaaagcttaaaagctctactcagatggaaattggttgaaaagagtaatttggaagagtcagatgatcctgatggctctcaaattgagaaaataaaagaagaaataactcagctgcgcaataatgtctttcaggaaatttatcatgagagagaggaatatgagatgctaactagtttggcacagaaatggttccctgagctgcctctgcttcatcctgaaataggattactcaaatacatgaactctggtggtctccttacaatgagcttggaacgagatcttcttgatgctgagcccatgaaggaacttagcagcaagcgtcctttggtacgttctgaggttaatgggcagataattctgttaaagggctattctgtggatgttgacacagaagccaaggtgattgagagagcagccacctaccatagagcttggagagaagctgaaggagactcagggttactgccattgatattcctgtttttatgtaagtctgatcctatggcttatctgatggtcccatactaccctagggcaaacctgaatgctgttcaagccaacatgcctttaaattcagaagaaactttaaaggtcatgaaaggtgttgcccagggtctgcatacattgcataaggctgacataattcatggatcacttcatcagaacaatgtatttgctttaaaccgtgaacaaggaattgttggagattttgacttcaccaaatctgtgagtcagcgagcctcggtgaacatgatggttggtgacttgagtttgatgtcacctgagttgaaaatgggaaaacctgcttctccaggttcagacttatatgcttatggctgcctcttattatggctttctgttcaaaatcaggagtttgagataaataaagatggaatccccaaagtggatcagtttcatctggatgataaagtcaaatccctcctctgtagcttgatatgttatagaagttcaatgactgctgaacaagttttaaatgctgaatgtttcttgatgccaaaggagcaatcagttccaaacccagaaaaagatactgaatacaccctatataaaaaggaagaagaaataaagacggagaacttggataaatgtatggagaagacaagaaatggtgaagccaactttgattgttaa

SEQ ID NO: 44 - DNA, 1275 bp, Homo sapiens
atgagcgtgggctgcccagagcctgagccgccccgctccctgacctgctgtgggccggggactgcccctgggcctggtgccggtgtgccccttctcactgaagacatgcaggccctgactctccgcacactggccgccagcgacgtcaccaagcactacgaactagtccgggagctgggcaaaggcacctatgggaaggttgacctggtggtctacaagggcacaggcacaaaaatggcactgaagtttgtgaacaagagcaaaaccaagctgaagaacttcctacgggaggtgagcatcaccaacagcctctcctccagccccttcatcatcaaggtctttgacgtggtctttgagacagaggactgctacgtctttgcccaggagtacgcacctgctggggacctgtttgacatcatccctccccaggtggggctccctgaggacacggtgaagcgctgtgtgcagcagctgggcctggcgctggacttcatgcacgggcggcagctggtgcaccgcgacatcaagcccgagaacgtgctgctgttcgaccgcgagtgccgccgcgtaaagctggccgacttcggcatgacgcgccgcgtgggctgccgcgtcaagcgcgtgagcggcaccatcccttacacggcgcctgaggtgtgccaggcgggccgcgccgacgggctggcggtggacacgggcgtggacgtgtgggccttcggcgtgctcatcttctgcgtgctcaccggcaacttcccgtgggaggcggcgtcgggcgccgacgccttcttcgaggagttcgtgcgctggcagcggggccgcctgccggggctgccttcgcagtggcgccgcttcaccgagcccgcgctgcgcatgttccagcgcttactggccctggagcccgagcgccgcggcccagccaaggaggtgttccgcttcctcaagcacgagctcacgtccgagctgcgccgccggccctcgcaccgcgcgcgcaagccccccggggaccgcccgcccgccgccgggccactgcgcctcgaggcgcctgggccgctcaagcggacggtgctgaccgagagcggcggcggctcccggcccgcgccccccgccgtcgggtcggtgcccttgcccgtgccggtgccggtgccagtgcccgtgccggtgcctgtgcccgagcccggcctagctccccaggggccccccggccggaccgacggccgcgcggacaagagcaaagggcaggtggtgctggccacggccatcgagatctgcgtctga

SEQ ID NO: 45 - DNA, 1947 bp, Homo sapiens
atggataagtacgatgtgattaaggccatcgggcaaggtgccttcgggaaagcatacttagctaaagggaaatcagatagcaagcactgtgtcataaaagagatcaattttgaaaagatgcccatacaagaaaaagaagcttcaaagaaagaagtgattcttctggaaaagatgaaacatcccaacattgtagccttcttcaattcatttcaagagaatggcaggctgtttattgtaatggaatattgtgatggaggggatctcatgaaaaggatcaatagacaacggggtgtgttatttagtgaagatcagatcctcggttggtttgtacagatttctctaggactaaaacatattcatgacaggaagatattacacagggacataaaagctcagaacatttttcttagcaagaacggaatggtggcaaagcttggggactttggtatagcaagagtcctgaataattccatggaacttgctcgaacttgtattggaacaccttactacctgtccccagagatctgtcagaataaaccctacaacaataaaacggatatttggtctcttggctgtgtcttatatgagctctgcacacttaaacatccttttgagggtaacaacttacagcagctggttctgaagatttgtcaagcacattttgccccaatatctccggggttttctcgtgagctccattccttgatatctcagctctttcaagtatctcctcgagaccgaccatccataaattccattttgaaaaggccctttttagagaatcttattcccaaatatttgactcctgaggtaagttttgaggtgactgtttggattttggcagagattttgggttgcaggtccttgacacgtgtgttcggttttaggtgtgagccactgcacccagcctgtattatggtttttaaaaacatcccctcttgttttcttcagataaaaatgatagaaagacccaaaattgctgctgtctgtggacattatgattattattatgctcaacttgatatgctgaggaggagagcccacaaaccaagttatcaccctattcctcaagaaaatactggagttgaggattacggtcaggaaacgaggcatggtccatccccaagtcaatggcctgctgagtaccttcagagaaaatttgaagctcaacaatataagttgaaagtggagaagcaattgggtcttcgtccatcttctgccgagccaaattacaaccagagacaagagctaagaagtaatggagaagagcctagattccaggagctgccatttaggaaaaacgaaatgaaggaacaggaatattggaagcagttagaggaaatacgccaacagtaccacaatgacatgaaagaaattagaaagaagatggggagagaaccagaggacattgaaaaagacttgaaacaaatgaggcttcagaacacaaaggaaagtaaaaatccagaacagaaatataaagctaagaagggggtaaaatttgaaattaatttagacaaatgtatttctgatgaaaacatcctccaagaggaagaggcaatggatataccaaatgaaactttgacctttgaggatggcatgaagtttaaggaatatgaatgtgtaaaggagcatggagattatacagacaaagcatttgaaaaacttcactgcccagaagcagggttttccacgcagactgtagctgctgtgggaaacaggaggcagtgggatggaggagcgcctcagactctgctgcagatgatggcagtggccgacatcacctccacctgccccacggggcctgacaatggccaagttattgtgattgaaggcattccaggaaacaggaaacagtggcggcatgaagctccaggaactttaatgagtgttttggcagcagcacatctaacgagtagctcattttctgccgatgaagaatttggtatg

SEQ ID NO: 46 - DNA, 1938 bp, Homo sapiens
atgctgaaattccaagaggcagctaagtgtgtgagtggatcaacagccatttccacttatccaaagaccttgattgcaagaagatacgtgcttcaacaaaaacttggcagtggaagttttggaactgtctatctggtttcagacaagaaagccaaacgaggagaggaattaaaggtacttaaggaaatatctgttggagaactaaatccaaatgaaactgtacaggccaatttggaagcccaactcctctccaagctggaccacccagccattgtcaagttccatgcaagttttgtggagcaagataatttctgcattatcacggagtactgtgagggccgagatctggacgataaaattcaggaatataaacaagctggaaaaatctttccagaaaatcaaataatagaatggtttatccagctgctgctgggagttgactacatgcatgagaggaggatacttcatcgagacttaaagtcaaagaatgtatttctgaaaaataatctccttaaaattggagattttggagtttctcgacttctaatgggatcctgtgacctggccacaactttaactggaactccccattatatgagtcctgaggctctgaaacaccaaggctatgacacaaagtcggacatctggtcactggcatgcattttgtatgagatgtgctgcatgaatcatgcattcgctggctccaatttcttatccattgttttaaaaattgttgaaggtgacacaccttctctccctgagagatatccaaaagaactaaatgccatcatggaaagcatgttgaacaagaatccttcattaagaccatctgctatcgaaattttaaaaatcccttaccttgatgagcagctacagaacctaatgtgtagatattcagaaatgactctggaagacaaaaatttggattgtcagaaggaggctgctcatataattaatgccatgcaaaaaaggatccacctgcagactctgagggcactgtcagaagtacagaaaatgacgccaagagaaaggatgcggctgaggaagctccaggcggctgatgagaaagccaggaagctgaaaaagattgtggaagaaaaatatgaagaaaatagcaaacgaatgcaagaattgagatctcggaactttcagcagctgagtgttgatgtactccatgaaaaaacacatttaaaaggaatggaagaaaaggaggagcaacctgagggaagactttcttgttcaccccaggacgaggatgaagagaggtggcaaggcagggaagaggaatctgatgaaccaactttagagaacctgcctgagtctcagcctattccttccatggacctccacgaacttgaatcaattgtagaggatgccacatctgaccttggataccatgagatcccagaagacccacttgtggctgaagagtactacgctgatgcatttgattcctattgtgtagagagtgatgaggaggaagaagaaatagcgttagaaagaccagagaaagaaatcaggaatgagggatcccagcctgcttacagaacaaaccaacaggacagtgatatcgaagcgttggccaggtgtttggaaaatgtcctgggttgcacttctctagacacaaagaccatcaccaccatggctgaagacatgtccccaggaccaccaattttcaacagtgtgatggccaggaccaagatgaaacgcatgagggaatcagccatgcagaagctggggacagaagtatttgaagaggtctataattacctcaagagagcaaggcatcagaatgctagcgaagcagagatccgcgagtgtttggaaaaagtggtgcctcaagccagcgactgttttgaagtggaccagctcctgtactttgaagagcagttgctgatcacgatgggaaaagaacctactctccagaaccatctctag

SEQ ID NO: 47 - DNA, 1338 bp, Homo sapiens
  modified_base (1)..(1338): “N” represents a, t, c, g, other or unknown
atgccgtcccgggctgagaactatgaagtgttgtacaccattggcacaggctcctgtggccgctgccagaagatccagaggaagagtgacggcaagatactagtttggaaagaacttcattatggctccatgacagaagctgagaaacagatgcttgtttctgaagtgaatttgctttgtgaactgaaaaatccaaacatcgttcattactatgatcgtattattgaccggaccaacacaacactgtacattgtaatggaatattgtgaagaaggagacctggctagtgtaattacaaagggaaccaaggaaaggcaatacttagatgaagagtttgttcttcgagtgacaactcagttgactctggccctgaagtnatgccacagacgaagtgatggtgatcatactgtagtgcgtcgggatctgaaaccagccagtgttttcctggatggcaagcaaaacgtcaagcttggagatttggggctagccagaatattaaaccacgacacgagttttgcaaaaacatttgttggcacaccttattacatgtctcctgaacaaacgaatcacatgtcctacaatgagaaaccagatatctggtcattgggctgcttgccgtatgagtcacgtgcattaatgcctccatttacagcttttagccagaaagaactcgctgggaaaatcagagaaggcaaattcaggcgaattctataccgttactctgatgaattgaatgaaattattatgaggatgttaaaggattaccatcgaccttctgttgaagaaattctcgagaaccctttaatagcagatttggttgcagaagagcaaagaagaaatcttgagagaagagggcgacaattaggagagccagaaaaattgccggattccagccctgtattgagtgagctgaaactaaaggaaattcagttagaggagcaagagcgagctctcaaagcaggagaagaaaggttggagcagaaagaacaggagctttgtgtttgtgagagactagcagaggacagactggctataccagaaaatctgttgaagaattacagcttgctaaaggaacagaagttcctgtctctggcaagtagtccagaacttcttaatcttccatcctcagtaattaagaagaaagttcatttcagtggggaaagtaaagagaatgtcatgaggagtgagaatcctgagagtcagctcacatctaagtccaagtgcaaggacctgaagnnnnnntgcttcatgcttcatgctgcccagctgcgggctcaagccctgtcagatattgagaaaaattatcaactaaaaagcagacagatcctgggcatgcgc

SEQ ID NO: 48 - DNA, 1829 bp, Homo sapiens
  modifed_base (1)..(1829): “N” represents a, t, c, g, other or unknown
taataatagtacctacctcatgtcatcgttttaaggattaaatgaattaacacatttaaaatataagaagtttgaaaagctcttggactccggctgcagcgtcagcctccctcgggcctcggcagcggcggcggctcgctcgcctcagccccagcgcccctcggctaccctcggaccaggcccgcagcgccgcccgccctcggccgccccgacgccggcctgggccgcggtcgcagccccgggctcgcgtaggcgccgaacgctcccggcccgcccactacgggcccaggctagaggcgccgccgccaccggcccgcggagcccggatgctggcccggaggaagccgatgctgccggcgctcaccatcaaccctaccatcgccgagggcccgtccccaaccagcgagggcgcctccgaggcaaacctggtggacctgcagaagaagctggaggagctggaacttgacgagcagcagaagcggctggaagcctttctcacccagaaagccaaggtcggcgaactcaaagacgatgacttcgaaaggacctcagagctggacgcgggcaacggcggggtggtcaccaaagtccagcacagaccctcgggcctcatcatggccaggaagctgatccaccttgagatcaagccggccatccggaaccagatcatccgcgagcaccaggtcctgcacgagtgcaactcaccgtacatcgtgggcttctacggggccttctactgtgacagggagatcagcatctgcatggagcacatggatggcggctccctggaccaggggctgaaagaggccaagaggattcccgaggacatcctggggaaagtcagcattgcggttctccggggcttggcgtacctccgagagaagcaccagatcatgcaccgaaatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggtcatctggagcatggacctgtccctggtggagctggccatcgaaaggtaccccatccccccgcccgacgccaaggagctggaggccatctttggccagcccgtggtcgacagggaagaaggagagcctcacagcatctcctcttggccagggtcccccgggcgccccaacagcggttacgggatggacagcctgcccgccatggccatcttcgaactgctggactatattgtgaaagagccgcctcctaagctgcccaacggtgtgttcacccccgacttccaggagtttgtcaataaatgcctcatcaaaaacccaacggagcgggcggacctaaagatgctcacaaaccacgccttcatcaagcggtccgaggtgaaagaagcggattttgcctgcnnnttgtgtaaaaccctggggctgaaccagcccggcacacccacgcgcaccgccgtgtgaccggcacacccacgcgcaccgccgtgtaacagtggccaggctccccgcgtcccgctggtgacctgcccaccgtccctgtccacgccccgcccttccagctgaggacaggctggcgcctccacccaccctcctgcctcacccctgcggagagcaccgtggcggggcgacaactgcgcacgcaggaacgggggtctcctctcctgctggtccctgccggcgtgcctctagggacgggcgacgctgctgtgtgtggtctcagagcctctgcttccttaggttacaaaacaaaacagggagagaaaa

SEQ ID NO: 49 - DNA, 834 bp, Homo sapiens
aatagtttaaagtctgaagaacctatcctatggaccaagggtgagattcttggaaagggagcctacggcacagtatactgtggtctcactagtcaaggacagctaatagctgtaaaacaggtggctttggatacctctaataaattagctgctgaaaaggaataccggaaactacaggaagaagtagatttgctcaaagcactgaaacatgtcaacattgtggcctatttggggacatgcttgcaagagaacactgtgagcattttcatggagtttgttcctggtggctcaatctctagtattataaaccgttttgggccattgcctgagatggtgttctgtaaatatacgaaacaaatacttcaaggtgttgcttatctccatgagaactgtgtggtacatcgcgatatcaaaggaaataatgttatgctcatgccaactggaataataaagctgattgactttggctgtgccaggcgtttggcctgggcaggtttaaatggcacccacagtgacatgcttaagtccatgcatgggactccatattggatggccccagaagtcatcaatgagtctggctatggacggaaatcagatatctggagcattggttgtactgtgtttgagatggctacagggaagcctccactggcttccatggacaggatggccgccatgttttacatcggagcacaccgagggctgatgcctcctttaccagaccacttctcagaaaatgcagcagactttgtgcgcatgtgcctgaccagggaccagcatgagcgaccttctgctctccagctcctgaagcactccttcttggagagaagtcac

SEQ ID NO: 50 - DNA, 84 bp, Homo sapiens
gatctaacccctcagaatatcctgcttgacaagagtgtctttgtatacctgggggtgtttggccttgccagagagtttatgcta

SEQ ID NO: 51 - DNA, 1044 bp, Homo sapiens
agagactgccctcccgttattgccccaccaccggatcatatgaaatcaatttacacacgggctgtaattgatcctgttcttgcaccagttggtgattcaaatgttgatggtggtgccaagtctttagacaaacagaaaaagaagactaagatgacagatgaagagattatggagaaactaagaactattgtgagcataggacccgtaagaaaaaaatatacaagatatgaaaaaattggacaaggggcttctggtacagttttcactgctaccgacgttgcactgggacagaaggttgctatcaaacaaattaatttacagaaacagccaaagaaggaattgatcattaatgagattctggtaatgaaagaattaaaaaatcccaacatagttaacttcttggacagttacctggtaggagatgaattgtttgtggtcgtggaataccttgctagggggtcactcactgatgtggtaacagaaacctgcatggatgaagcacagattgctgctgtatgcagagagagtttacaggcattggagtttttacatgctaatcaagtgatccacagagacatcaaaagtgacagtgtacttttgggaatggaaggatcagttaagctcactgactttggtttctgtgcccagatcacccctgagcagagcaaacgcagtaccgtggtcagaacgccacactggatggcaccagaagtggttacacggaaggcttatggccctaaagtcagtgtatggtctctgggtatcatggctactgagatggtagaaggagagcctccatacctcaatgaaaatcccttgagggccttgtgcctaatagcaactaatggaatcccagaacttcagaatccagagacactttccccaatatttcgggatttcttaaatcgatgtttggaaacagatgtggaaaaaaggggttcagccaaagaattattacagcatcttttcctgaaactagccaaactgttatctagcttgacaccactgatcatggcagctaaagaagcaatgaagagtaaccgt

SEQ ID NO: 52 - DNA, 3318 bp, Homo sapiens
atgatgcttggacttgaatcacttccagatcccacagacacctgggaaattatagagaccattggtaaaggcacctatggcaaagtctacaaggtaactaacaagagagatgggagcctggctgcagtgaaaattctggatccagtcagtgatatggatgaagaaattgaggcagaatacaacattttgcagttccttcctaatcatcccaatgttgtaaagttttatgggatgttttacaaagcggatcactgtgtagggggacagctgtggctggtcctggagctgtgtaatgggggctcagtcactgagcttgtcaaaggtctactcagatgtggccagcggttggatgaagcaatgatctcatacatcttgtacggggccctcttgggccttcagcatttgcacaacaaccgaatcatccaccgtgatgtgaaggggaataacattcttctgacaacagaaggaggagttaagctcgttgactttggtgtttcagctcaactcaccagtacacgtctgcggagaaacacatctgttggcaccccgttctggatggcccctgaggtcattgcctgtgagcagcagtatgactcttcctatgacgctcgctgtgacgtctggtccttggggatcacagctattgaactgggggatggagaccctcccctctttgacatgcatcctgtgaaaacactctttaagattccaagaaatcctccacctactttacttcatccagaaaaatggtgtgaagaattcaaccactttatttcacagtgtcttattaaggattttgaaaggcgaccttccgtcacacatctccttgaccacccatttattaaaggagtacatggaaaagttctgtttctgcaaaaacagctggccaaggttctccaagaccagaagcatcaaaatcctgttgctaaaaccaggcatgagaggatgcataccagaagaccttatcatgtggaagatgctgaaaaatactgccttgaggatgatttggtcaacctagaggttctggatgaggatacaattatccatcagttgcagaaacgttatgcagacttgctaatttacacatatgttggagacatcttaattgccttaaaccccttccagaatctaagcatatactctccacagttttccagactttatcatggggtgaaacgcgcctccaatcccccccacatatttgcatcagcagatgctgcttaccagtgcatggttactctcagcaaagaccagtgcattgtcatcagcggagagagtggctctgggaagacagaaagcgcccacctgattgttcagcatttgactttcttgggaaaggccaataatcagaccttgagagagaaaattctacaagtcaactccctggtggaagcctttgggaactcatgcactgccatcaatgacaactcgagccgttttggaaaatatctggaaatgatgtttacaccaactggagttgtgatgggggcaagaatctctgaatatctcctggaaaaatccagagttataaaacaggcagcgagagagaaaaattttcatatattttactatatttatgctggtcttcatcaccaaaagaagctttctgatttcagacttcctgaggaaaaacctcctaggtacatagctgatgaaactggaagggtgatgcacgacataacttccaaggagtcttacagaagacaattcgaagcaattcagcattgcttcaggattatagggttcacggacaaagaggtgcactcagtgtacagaattttggctgggattttgaatattgggaacattgagttcgcagctatttcctctcaacatcagactgataaaagtgaggtgcccaatgctgaagctttgcaaaatgctgcctctgttctgtgcattagccctgaagagctccaggaggccctcacctcccactgtgtggtcacccggggcgagaccatcatccgtgccaacactgtagacagggctgcggacgttcgagacgccatgtccaaagccctgtatgggaggctcttcagctggattgtgaatcgcattaatacactcctgcagccagacgaaaacatatggcaagttcctcggagagcagagggaggtggaatgaatgtggggatcttggatatctttggattcgagaattttcagagaaattcatttgagcagctctgcataaacatcgccaatgagcaaatccagtactatttcaatcagcatgtttttgctcttgagcagatggaatatcagaatgaaggcattgatgctatacccgtggaatatgaggacaaccgcccgctcctggacatgttcctccagaaacccctgggactgcttgcacttttggatgaggaaagtcggtttccccaagcaactgaccagaccctggttgataaatttgaagataatctacgatgcaaatacttctggaggcccaaaggagtggaactgtgctttggcattcagcattatgctggaaaggtattatatgatgcttctggggttcttgagaaaaatagagacactctccctgccgatgtggttgtggtcctgagaacgtcagaaaacaagcttcttcagcagctcttctcaatccctctgaccaaaacaggtacttgggaaccctctgatagccctgctcttaaagcttttgcaagaccaggtaatttggcccagacaagagctaggataacagtggcctcaagttctttgcctccacatttcagtgctgggaaagccaaggtggacactctggaggtgatacggcatccggaagaaaccaccaacatgaagaggcaaactgtggcttcttacttccggtattctctgatggacctgctctccaaaatggtggttggacagccccactttgtgcgctgcattaaacccaatgatgaccgagaggccctgcagttctctcgagagagggtgctggcccagctccgctccacagggattctggagacagtcagcatccgccgccagggctattcccaccgcatcctttttgaagaatttgtgaaaaggtattattacttggcattcacagcacatcaaacacctcttgctagcaaagagagctgtgtggctatcttggaaaagtccagattagatcactgggtgctgggaaaaacaaaggtttttctcaaatattaccatgttgagcaattaaatttgctgcttcgagaagtcataggcagagtggttgtgctgcaggcatataccaaggggtggcttggagccaggagatacaaaaaggtcagagagaagagagagaagggagccattgccatccagtca

SEQ ID NO: 53 - DNA, 3112 bp, Homo sapiens
gggggcattgctcagcggtgctaggctggcgcggcttgagccgccgccggactgacagctcggtctgcggaccatggagacctgcgccggtccacacccgctgcgcctcttcctctgccggatgcagctctgtctcgcgctgcttttgggaccctggcggcctgggaccgccgaggaagttatcctcctggattccaaagcctcccaggccgagctgggctggactgcactgccaagtaatgggtgggaggagatcagcggcgtggatgaacacgaccgtcccatccgcacgtaccaagtgtgcaatgtgctggagcccaaccaggacaactggctgcagactggctggataagccgtggccgcgggcagcgcatcttcgtggaactgcagttcacactccgtgactgcagcagcatccctggcgccgcgggtacctgcaaggagaccttcaacgtctactacctggaaactgaggccgacctgggccgtgggcgtccccgcctaggcggcagccggccccgcaaaatcgacacgatcgcggcggacgagagcttcacgcagggcgacctgggtgagcgcaagatgaagctgaacacagaggtgcgcgagatcggaccgctcagccggcggggtttccacctggcctttcaggacgtgggcgcatgcgtggcgcttgtctcggtgcgcgtctactacaagcagtgccgcgccaccgtgcggggcctggccacgttcccagccaccgcagccgagagcgccttctccacactggtggaagtggccggaacgtgcgtggcgcactcggaaggggagcctggcagccccccacgcatgcactgcggcgccgacggcgagtggctggtgcctgtgggccgctgcagctgcagcgcgggattccaggagcgtggtgacatctgcgaagcctgtcccccagggttttacaaggtgtccccgcggcgaagggtctgctcaccgtgcccagagcacagccgggccctggaaaacgcctccaccttctgcgtgtgccaggacagctatgcgcgctcacccaccgacccgccctcggcttcctgcacccgtgggccgccgtcggcgccgcgggacctgcagtacagcctgagccgctcgccgctggtgctgcgactgcgctggctgccgccggccgactcgggaggccgctcggacgtcacctactcgctgctgtgcctgcgctgcggccgcgagggcccggcgggcgcctgcgagccgtgcgggccgcgcgtggccttcctaccgcgccaggcagggctgcgggagcgagccgccacgctgctgcacctgcggcccggggcgcgctacaccgtgcgcgtggccgtgctcaacggcgtctcgggcccggcggccgccctggttccggttggcgctgtttcaattaaccctggtacggttggccctgttcctgttgccggggttatccgcgaccgagtggaaccccagagcgtgtccctgtcgtggcgggagcccatccctgccggagcccctggggccaatgacacggagtacgagatccgatactacgagaaggtgcagagtgagcagacttactccatggtgaagacaggggcgcccacagtcaccgtcaccaacctgaagccggctacccgctacgtctttcagatccgggccgcttccccggggccatcctgggaggcccagagttttaaccccagcattgaagtacagaccctgggggaggctgcctcagggtccagggaccagagccccgccattgtcgtcaccgtagtgaccatctcggccctcctcgtcctgggctccgtgatgagtgtgctggccatttggaggaggccctgcagctatggcaaaggaggaggggatgcccatgatgaagaggagctgtatttccacttcaaagtcccaacacgtcgcacattcctggacccccagagctgtggggacctgctgcaggctgtgcatctgttcgccaaggaactggatgcgaaaagcgtcacgctggagaggagccttggaggagggcggtttggggagctgtgctgtggctgcttgcagctccccggtcgccaggagctgctcgtagccgtgcacatgctgagggacagcgcctccgactcacagaggctcggcttcctggccgaggccctcacgctgggccagtttgaccatagccacatcgtgcggctggagggcgttgttacccgaggaagcaccttgatgattgtcaccgagtacatgagccatggggccctggacggcttcctcaggcggcacgaggggcagctggtggctgggcaactgatggggttgctgcctgggctggcatcagccatgaagtatctgtcagagatgggctacgttcaccggggcctggcagctcgccatgtgctggtcagcagcgaccttgtctgcaagatctctggcttcgggcggggcccccgggaccgatcagaggctgtctacaccactatgagtggccggagcccagcgctatgggccgctcccgagacacttcagtttggccacttcagctctgccagtgacgtgtggagcttcggcatcatcatgtgggaggtgatggcctttggggagcggccttactgggacatgtctggccaagacgtgatcaaggctgtggaggatggcttccggctgccaccccccaggaactgtcctaaccttctgcaccgactaatgctcgactgctggcagaaggacccaggtgagcggcccaggttctcccagatccacagcatcctgagcaagatggtgcaggacccagagccccccaagtgtgccctgactacctgtcccaggcctcccaccccactagcggaccgtgccttctccaccttcccctcctttggctctgtgggcgcgtggctggaggccctggacctgtgccgctacaaggacagcttcgcggctgctggctatgggagcctggaggccgtggccgagatgactgcccagagggacctggtgagcctaggcatctctttggctgaacatcgagaggccctcctcagcgggatcagcgccctgcaggcacgagtgctccagctgcagggccagggggtgcaggtgtgagtggacccc

SEQ ID NO: 54 - DNA, 2730 bp, Homo sapiens
atgcagcccctggactttagttcagggggaagtgaccccaacatcagcctctcagaaaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacaggatgttagcagcatttttggcctttatattcgagacattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacccgcctagaaatattatgttgaataatgatgagttggaatttgaacaagctccagagtttctcctagactgttttgtgtgtattcacttatatccatcaagtgactacatttcaaggcactatatgagaaccataaatattgtacaaacaggatttgctaaatgtcggtggagagtaacagtccacggggctgatcatggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaaggtaa

SEQ ID NO: 55 - DNA, 3495 bp, Homo sapiens
atggcaggggccggaggccagcaccaccctccgggcgccgctggaggagcggccgccggagccggcgccgcggtcacctccgccgctgcctcggcggggccgggagaggattcgtctgacagcgaagcggagcaagagggaccccagaaactgatccgcaaagtgtctacctcggggcagatccggaccaagaccagtattaaagagggacagctattgaagcaaaccagttctttccaaaggtggaaaaagcgatacttcaaacttcgaggccgcaccctttactatgcaaaggactcaaagtctctgatatttgatgaagttgacctctcagatgctagtgtagctgaagcaagcacgaaaaatgctaacaacagcttcacgatcatcactccattcagaaggctaatgctgtgtgctgagaacagaaaggagatggaggattggatcagctcactgaagtctgtacagaccagagaaccctacgaggtggcccagtttaatgtggaacatttctcagggatgcacaactggtacgcctgctcccacgcccgacccaccttctgtaacgtgtgcagagagagtctttctggagtcacctcccatggcctgtcctgcgaagtgtgtaaattcaaggctcacaaaagatgtgcagtgagagcaacaaataactgtaaatggactaccctggcctccatcgggaaggacattatagaagatgaagatggcgtcgcgatgcctcaccagtggcttgagggcaacctgcctgtaagtgccaagtgtgctgtctgcgacaaaacatgtggcagtgttctccgtctacaggattggaaatgcctttggtgtaagacaatggtacacactgcctgcaaagatttataccatccaatatgtccacttggtcaatgtaaagtatctatcatacctccaattgcactaaacagcaccgattccgatggtttctgtagagcaacattttcgttctgtgttagtcctctattggtttttgtcaattctaagagtggagataatcagggagtaaagttcctccgtcgctttaaacagttgctaaatccggctcaggtgtttgatttaatgaatggaggtcctcatttaggtttaagattatttcagaagtttgacaatttccggattcttgtatgtggaggcgatggaagtgtaggttgggttttgtcagaaatcgataagctcaacttgaataaacagtgtcagctgggagtgttgcctttgggtacaggaaatgaccttgcccgagttcttggctggggaggttcatatgacgatgacacccagcttcctcagatcctagagaaactggaacgagccagtaccaaaatgttggacaggtggagtataatgacatatgaactcaaattgccaccaaaagcttccctacttccaggacctccagaagcatctgaagaattttatatgacgatttatgaagactcagttgcaacgcatcttacaaaaatcctcaattctgatgaacatgcagtggtcatatcttctgccaagacgctatgtgaaactgtaaaggacttcgttgccaaagtagaaaagacgtatgacaaaaccttggaaaatgccgttgtagctgatgccgtggccagtaaatgttcagtcctaaacgagaagctcgaacaactgctgcaggctttgcacacagattcccaggctgcgcctgttctccctggcctcagccctctcattgtggaagaagatgctgtggaatcgtccagtgaagagtccctgggtgaaagcaaagagcagcttggggatgacgttacaaaaccttcctcccagaaagccgtcaaaccaagggaaatcatgttgcgggcaaatagtttaaagaaagcagtgaggcaagtcattgaggaagccggaaaagttatggatgacccgacagttcacccctgtgaaccagctaatcagtcctctgattatgacagcacagaaacagatgaatctaaggaggaagctaaagatgatggtgccaaagaatcaataactgttaaaactgcacctcggtctccagatgcccgggcaagttatggccattcccaaactgattctgtccctggtccagctgtggcagccagcaaagaaaacctccctgtgctcaataccagaataatctgcccaggtttaagagcaggactggctgcctcaattgctgggagttcgattatcaacaaaatgttactggcaaacattgatccttttggtgccacgccgtttattgaccctgatctagattccgtagatggatattcagaaaaatgtgtcatgaacaattactttgggattggattagatgcaaaaatttcattagaatttaataataaaagagaggagcaccctgaaaaatgcaggagccgaactaaaaacttgatgtggtatggagtccttggaacccgggagttattacagagatcgtacaagaatttagaacaaagggttcaacttgagtgtgatgggcagtatattcctcttcccagcttgcaaggcatagccgtgttgaacattcccagctatgctggaggcactaacttttggggtggaactaaagaggatgatatatttgctgcaccatcctttgatgacaagatcctggaagttgtagcaatatttgatagcatgcaaatggcagtttcaagggtcattaaactgcagcatcatcgaatagcccagtgccgtacagtgaaaatcactatatttggtgacgaaggagtcccagtgcaagtggatggtgaagcgtgggttcagcctccagggattatcaaaattgtgcacaaaaacagagcacaaatgctaacaagggacagagcctttgagagcactctgaaatcttgggaagataagcagaagtgtgattctggtaaaccagttctccgaacccatttgtacatccatcacgccattgacttggcaacagaagaggtgtcgcagatgcagctatgctcccaggctgcagaggagctcattactaggatatgtgacgcagccacaattcactgtcttttggagcaagaactggcccatgctgtgaatgcctgctcccatgccctgaataaagccaacccaaggtgcccggagagtcttacaagagacactgccactgaaatagccatcaatgtgaaggcgctgtataatgaaacagaatctttgctagttggcagggttcctttgcagctggaatcgccacatgaagagcgagtatccaatgccttacactctgtggaggtggaattacagaaactgacagagattccttggctttattatatcttacacccaaatgaagatgaggaacctcctatggattgcaccaaaaggaacaacagaagcaccgtatttcgaatagtgccaaagtttaaaaaggaaaaggttcagaagcagaagacaagttcacagcctggatctggggataccgaaagtgggtcatgtgaagcgaattctccagggaattaa

SEQ ID NO: 56 - DNA, 2652 bp, Homo sapiens
atggctctcaaaccatgtcttgaggccccaattgagtctgggctctgttcagggattgaatgtcatgctgagcacccaacactagttctgatgctctttgccagtgtgctggtgacttgccttgaggctgctaaactcaccgtaggcttccagaccccctggaacatctcccatccattcagcatgcaaaggctgggtgcaggcctccagattgccatggacaaggtcaactcagagctagtggaccttggaaatttcaccacttacaccaactcagcctgcagcaccaaggagtctcttgctattttcaacaaccaggtccagaacgaacagatttctgccctgtttggaccagcatgcccagaagcagctgaggacattggtgatgtgctccaggaaagtctccagtacctgggctggaaacacattgggatgtttggaggctactctggggcttcctcctgggatggagtggatgaactgtggagggttgtagagaatgaactcaaatcccattttatcatcactgccagcatgagatacaccaacaatagtctagtccttcttcaagagcatctttggaggatatcatcaattgccagggttatcatcttaacgtgcagctcagaggatgcaaaaattattcttctggctgcagcaaacctgggactcaacactggagaatttgttttcatcattttgcagcagttggaggtgggtaccgttgaccactcaatggctctggtagtaagaggaccgtttttggaaggaagtactgacaaatcagaaaatgatgcacctccaaaaggtgtatgggtcactgcttctcattgccctcagctcctacagaaaaggccgtggagacgaaggcttttggaaacaagtctaccaaacaccgaggaggtcgcccttccgcagcaccatctcctgggaggagcaggaggatgccaggacctagacccaggatggagtggtcagagatgtggagctaggacaggtcagggccagcaggtgagcccttactctgcctaccttcacgacgccgtcctgctctatgctgagaccgtgaagcaggtggtaaaggctggaggcgacttccaggatgggtggcagctggtcagcgctctgaagggttccagtcagaccacagtgcagggaatcacaggccctgtgtttgtggatgcccagggagaaaggcacatggattactctgtctatgccctgcagaagtctgaaaatgggccccttttactttcttttcttcattatgacagttatcaaagcgtgactgctgtgcttctgacattgatgatcctcattcctgtcttgggagctgccatcataggtctgattttaaggatgcagaggcaaaacaaagacatctggtggcaaatcaattttgatgatatcaccattcttccccagaacaagccatcccagagagccacacctgtgtcaaaaggcatcaacagtaactcatctagtgtgatgatttctgtggacctcagctcttttgtcaagagccagcagtgggaagagctcttctatgccgcagtagggctttatcagggaaaccatgtggccatccgttacgttggtgaccaagcagaagcctgggttaggaagccgattgtgctacaggaaatacagctgcttgctgcctatacttttaccaccagaagcgacaaagccagtgacctgggagtcagtccgggcatgctgttcctccacaggagccccctgggctcccacagcaacctgaaaccttccaactgcctgatggatggtcggctgcagtaccctcactccatgcggacatcccattcttttgcagagctctactggactgccccagagctgctgcagttcccggagatgccctggtcgggtaccccgcaaggagatgtttacagcttcgccattctgatgagggagctgatctaccattgggaccacgggccttttgatgacctccacgaggcaccggatgaaatcatcaaccaaatcaaagaccctgcagcagcagtcccactgcaaccttccctgcccgaggagaagggcaatgaaaagatcgtggccatggtgagggtgtgttgggatgaatctctggagaaaagacccagtttctcttccatcaagaaaactttacgagaggccagtcccagaggtcatgtgagcatactggccagtgtgatgagcaagctggaagtgtatgccaattacctggaggaagtggtgcaagagaggaccagccagctgactgcagagaagaggaaggtggagaagcttctgtccaccaaagtgcccagcttcactggagaacaactactagccggaaggtccgtggaaccagaacatttcgaatctgtgacaatctttttatctgatattgttggattcacaaagctgtgttctctcagctcccctttgcaagtcgtcaagctcctcaatgacgtgtacagtttattcgatcacatcatcacaacttatgatgtttataagggcaaaggagagcaaacaactttctggttgaaagataaagaaggcttcactcttccactcccgaatttactgaggaaaaagccaaagtcccagagatattgtgagctaatcagcttgcaaggagataggagctcatatctgacagcatct

SEQ ID NO: 57 - DNA, 144 bp, Homo sapiens
agagaattagatcatccaaacatctgcagattcactggaggctgtattgcactgccagatgtggttattgtgatggaatactgcccaaagggaagcttgatggatgttctgctcaatgataacatctctttcaactggggattt

SEQ ID NO: 58 - protein, 2053 aa, Homo sapiens
MLKFKYGARNPLDAGAAEPIASRASRLNLFFQGKPPFMTQQQMSPLSREGILDALFVLFEECSQPALMKIKHVSNFVPEVYSDTIAELQELQPSAKDFEVRSLVGCGHFAEVQVVREKATGDIYAMKVMKKKALLAQEQVSFFEEERNILSRSTSPWIPQLQYAFQDKNHLYLVMEYQPGGDLLSLLNRYEDQLDENLIQFYLAELILAVHSVHLMGYVHRDIKPENILVDRTGHIKLVDFGSAAKMNSNKMVNAKLPIGTPDYMAPEVLTVMNGDGKGTYGLDCDWWSVGVIAYEMIYGRSPFAEGTSARTFNNIMNFQRFLKFPDDPKVSSDFLDLIQSLLCGQKERLKFEGLCCHPFFSKIDWNNIRNSPPPFVPTLKSDDDTSNFDEPEKNSWVSSSPCQLSPSGFSGEELPFVGFSYSKALGILGRSESVVSGLDSPAKTSSMEKKLLIKSKELQDSQDKCHKMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKATECQHKLLKAKDQGKPEVGEYAKLEKINAEQQLKIQELQEKLEKAVKASTEATELLQNIRQAKERAERELEKLQNREDSSEGIRKKLVEAEERRHSLENKVKRLETMERRENRLKDDIQTKSQQIQQMADKILELEEKHREAQVSAQHLEVHLKQKEQHYEEKIKVLDNQIKKDLADKETLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKNRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVMDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVRQHS

SEQ ID NO: 59 - protein, 553 aa, Homo sapiens
MVDMGALDNLIANTAYLQARKPSDCDSKELQRRRRSLALPGLQGCAELRQKLSLNFHSLCEQQPIGRRLFRDFLATVPTFRKAATFLEDVQNWELAEEGPTKDSALQGLVATCASAPAPGNPQPFLSQAVATKCQAATTEEERVAAVTLAKAEAMAFLQEQPFKDFVTSAFYDKFLQWKLFEMQPVSDKYFTEFRVLGKGGFGEVCAVQVKNTGKMYACKKLDKKRLKKKGGEKMALLEKEILEKVSSPFIVSLAYAFESKTHLCLVMSLMNGGDLKFHIYNVGTRGLDMSRVIFYSAQIACGMLHLHELGIVYRDMKPENGLLDDLGNCRLSDLGLAVEMKGGKPITQRAGTNGYMAPEILMEKVSYSYPVDWFAMGCSIYEMVAGRTPFKDYKEKVSKEDLKQRTLQDEVKFQHDNFTEEAKDICRLFLAKKPEQRLRSREKSDDPRKHHFFKTINFPRLEAGLIEPPFVPDPSVVYAKDIAEIDDFSEVRGVEFDDKDKQFFKNFATGAVPIAWQEEIIETGLFEELNDPNRPTGCEEGNSSKSGVCLLL

SEQ ID NO: 60 - protein, 2523 aa, Homo sapiens
MQTKHFLVVTHVTPKHLHPSTCSAVTSYTQCSVHACSPPAPSGAASPPSSCRTSNRKSLIGNGQSPALPRPHSPLSAHAESLFEPYMRIVCNLLTLRFSYISSTPLNLATRSLDEILNWTVEIALKIVQEISPPVPQPIFHLHGDAILHLPGLLHPSHMHSLSSSAQEKHRENKMAPSAKFLNIQDKLGLWSECPRYLFSFPQRPNYILKWIKEKLHQLPYQPTPDELHFLSKHFCTTESIATENRCRNTPMRPRSRSLRHCISKPINLEEKAQLSCSPVRLATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSVGSFSEHLDQINGRSECVDSTDNSSKPSSEPASHMARQRLESTEKKKISGKVTKSLSASALSLMIPGDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGPVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLEPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 61 - protein, 327 aa, Homo sapiens
MGANTSRKPPVFDENEDVNFDHFEILRAIGKGSFGKVCIVQKNDTKKMYAMKYMNKQKCVERNEVRNVFKELQIMQGLEHPFLVNLWYSFQDEEDMFMVVDLLLGGDLRYHLQQNVHFKEETVKLFICELVMALDYLQNQRIIHRDMKPDNILLDEHGHVHITDFNIAAMLPRETQITTMAGTKPYMAPEMFSSRKGAGYSFAVDWWSLGVTAYELLRGRRPYHIRSSTSSKEIVHTFETTVVTYPSAWSQEMVSLLKKVRRKTACPNEVTKGSRLSGLSLEVSIQYWGHQLSSLQKGRLNCDPTFELEEMILESKPLHKKKKRLAK

SEQ ID NO: 62 - protein, 631 aa, Homo sapiens [sequence omitted]

SEQ ID NO: 63 - protein, 572 aa, Homo sapiens [sequence omitted]

SEQ ID NO: 64 - protein, 373 aa, Homo sapiens
MHPHSGLGAPGLLPQTGAGGASVAVTPNLSRTQKQVARVREDTATALQRLVELTTSRVTPVRSLRDQYHLIRKLGSGSYGRVLLAQPHQGGPAVALKLLRRDLVLRSTFLREFCVGRCVSAHPGLLQTLAGPLQTPRYFAFAQEYAPCGDLSGMLQERGLPELLVKRVVAQLAGALDFLHSRGLVHADVKPDNVLVFDPVCSRVALGDLGLTRPEGSPTPAPPVPLPTAPPELCLLLPPDTLPLRPAVDSWGLGVLLFCAATACFPWDVALAPNPEFEAFAGWVTTKPQPPQPPPPWDQFAPPALALLQGLLDLDPETRSPPLAVLDFLGDDWGLQGNREGPGVLGSAVSYEDREEGGSSLEEWTDEGDDSKS

SEQ ID NO: 65 - protein, 357 aa, Homo sapiens
MARENGESSSSWKKQAEDIKKIFEFKETLGTGAFSEVVLAEEKATGKLFAVKCIPKKALKGKESSIENEIAVLRKIKHENIVALEDIYESPNHLYLVMQLVSGGELFDRIVEKGFYTEKDASTLIRQVLDAVYYLHRMGIVHRDLKPENLLYYSQDEESKIMISDFGLSKMEGKGDVMSTACGTPGYVAPEVLAQKPYSKAVDCWSIGVIAYILLCGYPPFYDENDSKLFEQILKAEYEFDSPYWDDISDSAKDFIRNLMEKDPNKRYTCEQAARHPWIAGDTALNKNIHESVSAQIRKNFAKSKWRQAFNATAVVRHMRKLHLGSSLDSSNASVSSSLSLASQKDCAYVAKPESLS

SEQ ID NO: 66 - protein, 358 aa, Homo sapiens
MRATPLAAPAGSLSRKKRLELDDNLDTERPVQKRARSGPQPRLPPCLLPLSPPTAPDRATAVATASRLGPYVLLEPEEGGRAYQALHCPTGTEYTCKVYPVQEALAVLEPYARLPPHKHVARPTEVLAGTQLLYAFFTRTHGDMHSLVRSRHRIPEPEAAVLFRQMATALAHCHQHGLVLRDLKLCRFVFADRERKKLVLENLEDSCVLTGPDDSLWDKHACPAYVGPEILSSRASYSGKAADVWSLGVALFTMLAGHYPFQDSEPVLLFGKIRRGAYALPAGLSAPARCLVRCLLRREPAERLTATGILLHPWLRQDPMPLAPTRSHLWEAAQVVPDGLGLDEAREEEGDREVVLYG

SEQ ID NO: 67 - protein, 513 aa, Homo sapiens
MEGGPAVCCQDPRAELVERVAAIDVTHLEEADGGPEPTRNGVDPPPRARAASVIPGSTSRLLPARPSLSARKLSLQERPAGSYLEAQAGPYATGPASHISPRAWRRPTIESHHVAISDAEDCVQLNQYKLQSEIGKVGLTDAYLQGAYGVVRLAYNESEDRHYAMKVLSKKKLLKQYGFPRRPPPRGSQAAQGGPAKQLLPLERVYQEIAILKKLDHVNVVKLIEVLDDPAEDNLYLVDLLRKGPVMEVPCDKPFSEEQARLYLRDVILGLEYLHCQKIVHRDIKPSNLLLGDDGHVKIADFGVSNQFEGNDAQLSSTAGTPAFMAPEAISDSGQSFSGKALDVWATGVTLYCFVYGKCPFIDDFILALHRKIKNEPVVFPEEPEISEELKDLILKMLDKNPETRIGVPDIKLHPWVTKNGEEPLPSEEEHCSVVEVTEEEVKNSVRLIPSWTTVILVKSMLRKRSFGNPFEPQARREERSMSAPGNLLVKEGFGEGGKSPELPGVQEDEAAS

SEQ ID NO: 68 - protein, 343 aa, Homo sapiens
MLLLKKHTEDISSVYEIRERLGSGAFSEVVLAQERGSAHLVALKCIPKKALRGKEALVENEIAVLRRISHPNIVALEDVHESPSHLYLAMELVTGGELFDRIMERGSYTEKDASHLVGQVLGAVSYLHSLGIVHRDLKPENLLYATPFEDSKIMVSDFGLSKIQAGNMLGTACGTPGYVAPELLEQKPYGKAVDVWALGVISYILLCGYPPFYDESDPELFSQILRASYEFDSPFWGDISESAKDFIRHLLERDPQKRFTCQQALRHLWVSGDTAFDRDILGSVSEQIRKNFARTHWKRAFNATSFLRHIRKLGQIPEGEGASEQGMARHSHSGLRAGQPPKW

SEQ ID NO: 69 - protein, 499 aa, Homo sapiens [sequence omitted]

SEQ ID NO: 70 - protein, 436 aa, Homo sapiens
MTAVYMNGGGLVNPHYARWDRRDSVESGCQTESSKEGEEGQPRQLTPFEKLTQDMSQDEKVVREITLGKRIGFYRIRGEIGSGNFSQVKLGIHSLTKEKVAIKILDKTKLDQKTQRLLSREISSMEKLHHPNIIRLYEVVETLSKLHLVMEYAGGGELFGKISTEGKLSEPESKLIFSQIVSAVKHMHENQIIHRDLKAENVFYTSNTCVKVGDFGFSTVSKKGEMLNTFCGSPPYAAPELFRDEHYIGIYVDIWALGVLLYFMVTGTMPFRAETVAKLKKSILEGTYSVPPHVSEPCHRLIRGVLQQIPTERYGIDCIMNDEWMQGVPYPTPLEPFQLDPKHLSETSTLKEEENEVKSTLEHLGITEEHIRNNQGRDARSSITGVYRIILHRVQRKKALESVPVMMLPDPKERDLKKGSRVYRGIRHTSKFCSIL

SEQ ID NO: 71 - protein, 75 aa, Homo sapiens
  modified-base (17): any amino acid
EEEEAQTMFGQILLAMXYCLYKNVAHADVSLQIILLPEERNIKIVDFGFSMTFGERRFGNPFCGMYPYVATELFL

SEQ ID NO: 72 - protein, 39 aa, Homo sapiens
FRQILSGLQYCRLKNITHPELKPQTHLVIEEGNIKITDF

SEQ ID NO: 73 - protein, 84 aa, Homo sapiens
NIFFDKERNIKIANYGFSSTFAKRDNTEKGTFDGTLPYAGPELFLGHGYQCPAINVWSLGIIVCKMVAGALPFYEQNCKDILKK

SEQ ID NO: 74 - protein, 794 aa, Homo sapiens
MVAGLTLGKGPESPDGDVSVPERKDEVAGGGGEEEEAEERGRHAQYVGPYRLEKTLGKGQTGLVKLGVHCITGQKVAIKIVNREKLSESVLMKVEREIAILKLIEHPHVLKLHDVYENKKYLYLVLEHVSGGELFDYLVKKGRLTPKEARKFFRQIVSALDFCHSYSICHRDLKPENLLLDEKNNIRIADFGMASLQVGDSLLETSCGSPHYACPEVIKGEKYDGRRADMWSCGVILFALLVGALPFDDDNLRQLLEKVKRGVFHMPHFIPPDCQSLLRGMIEVEPEKRLSLEQIQKHPWYLGGKHEPDPCLEPAPGRRVAMRSLPSNGELDPDVLESMASLGCFRDRERLHRELRSEEENQEKMIYYLLLDRKERYPSCEDQDLPPRNDVDPPRKRVDSPMLSRHGKRRPERKSMEVLSITDAGGGGSPVPTRRALEMAQHSQRSRSVSGASTGLSSSPLSSPRSPVFSFSPEPGAGDEARGGGSPTSKTQTLPSRGPRGGGAGEQPPPPSARSTPLPGPPGSPRSSGGTPLHSPLHTPRASPTGTPGTTPPPSPGGGVGGAAWRSRLNSIRNSFLGSPRFHRRKMQVPTAEEMSSLTPESSPELAKRSWFGNFISLDKEEQIFLVLKDKPLSSIKADIVHAFLSIPSLSHSVLSQTSFRAEYKASGGPSVFQKPVRFQVDISSSEGPEPSPRRDGSGGGGIYSVTFTLISGPSRRFKRVVETIQAQLLSTHDQPSVQALADEKNGAQTRPAGAPPRSLQPPPGRPDPELSSSPRRGPPKDKKLLATNGTPLP

SEQ ID NO: 75 - protein, 786 aa, Homo sapiens
MVIMSEFSADPAGQGQGQQKPLRVGFYDIERTLGKGNFAVVKLARHRVTKTQVAIKIIDKTRLDSSNLEKIYREVQLMKLLNHPHIIKLYQVMETKDMLYIVTEFAKNGEMFDYLTSNGHLSENEARKKFWQILSAVEYCHDHHIVHRDLKTENLLLDGNMDIKLAGTEDFGFGNFYKSGEPLSTWCGSPPYAAPEVFEGKEYEGPQLDIWSLGVVLYVLVCGSLPFDGPNLPTLRQRVLEGRFRIPFFMSQDCESLIRRMLVVDPARRITIAQIRQHRWMRAEPCLPGPACPAFSAHSYTSNLGDYDEQALGIMQTLGVDRQRTVESLQNSSYNHFAAIYYLLLERLKEYRNAQCARPGPARQPRPRSSDLSGLEVPQEGLSTDPFRPALLCPQPQTLVQSVLQAEMDCELQSSLQWPLFFPVDASCSGVFRPRPVSPSSLLDTAISEEARQGPGLEEEQDTQESLPSSTGRRHTLAEVSTRLSPLTAPCIVVSPSTTASPAEGTSSDSCLTFSASKSPAGLSGTPATQGLLGACSPVRLASPFLGSQSATPVLQAQGGLGGAVLLPVSFQEGRRASDTSLTQGLKAFRQQLRKTTRTKGFLGLNKIKGLARQVCQAPASRASRGGLSPFHAPAQSPGLHGGAAGSREGWSLLEEVLEQQRLLQLQHHPAAAPGCSQAPQPAPAPFVIAPCDGPGAAPLPSTLLTSGLPLLPPPLLQTGASPVASAAQLLDTHLHIGTGPTALPAVPPPRLARLAPGCEPLGLLQGDCEMEDLMPCSLGTFVLVQ

SEQ ID NO: 76 - protein, 262 aa, Homo sapiens [sequence omitted]

SEQ ID NO: 77 - protein, 765 aa, Homo sapiens
MAGFKRGYDGKIAGLYDLDKTLGRGHFACGTCSRHVFTGEKVAVKVIDKTKLDTLATGHLFQEVRCMKLVQHPNIVRLYEVIDTQTKLYLILELGDGGDMFDYIMKHEEGLNEDLAKKYFAQIVHAISYCHKLHVVHRDLKPENVVFFEKQGLVKLTDFGFSNKFQPGKKLTTSCGSLAYSAPEILLGDEYDAPAVDIWSLGVILFMLVCGQPPFQEANDSETLTMIMDCKYTVPSHVSKECKDLITRMLQRDPKRRASLEEIENHPWLQGVDPSPATKYNIPLVSYKNLSEEEHNSIIQRMVLGDIADRDAIVEALETNRYNHITATYFLLAERILREKQEKEIQTRSASPSNIKAQFRQSWPTKIDVPQDLEDDLTATPLSHATVPQSPARAADSVLNGHRSKGLCDSAKKDDLPELAGPALSTVPPASLKPTASGRKCLFRVEEDEEEDEEDKKPMSLSTQVVLRRKPSVTIRLTSRKSAPVLNQIFEEGESDDEFDMDENLPPKLSRLKMNIASPGTVHKRYHRRKSQGRGSSCSSSETSDDDSESRRRLDKDSGFTYSWHRRDSSEGPPGSEGDGGGQSKPSNASGGVDKASPSENNAGGGSPSSGSGGNPTNTSGTTRRCAGPSNSMQLASRSAGELVESLKLMSLCLGSQLHGSTKYIIDPQNGLSFSSVKVQEKSTWKMCISSTGNAGQVPAVGGIKFFSDHMADTTTELERIKSKNLKNNVLQLPLCEKTISVNIQRNPKEGLLCASSPASCCHVI

SEQ ID NO: 78 - protein, 612 aa, Homo sapiens
MATENGAVELGIQNPSTGKERRVDPDGCLTSSADKAPKGPTGERPLAAGKDPGPPDPKKAPDPPTLKKDAKAPASEKGDGTLAQPSTSSQGPKGEGDRGGGPAEGSAGPPAALPQQTATPETSVKKPKAEQGASGSQDPGKPRVGKKAAEGQAAARRGSPAFLHSPSCPAIISSSEKLLAKKPPSEASELTFEGVPMTHSPTDPRPAKAEEGKNILAESQKEVGEKTPGQAGQAKMQGDTSRGIEFQAVPSEKSEVGQALCLTAREEDCFQILDDCPPPPAPFPHRMVELRTGNVSSEFSMNSKEALGGGKFGAVCTCMEKATGLKLAAKVIKKQTPKDKEMVLLEIEVMNQLNHRNLIQLYAAIETPHEIVLFMEYIEGGELFERIVDEDYHLTEVDTMVFVRQICDGILFMHKMRVLHLDLKPENILCVNTTGHLVKIIDFGLARRYNPNEKLKVNFGTPEFLSPEVVNYDQISDKTDMWSMGVITYMLLSGLSPFLGDDDTETLNNVLSGNWYFDEETFEAVSDEAKDFVSNLIVKDQRARMNAAQCLAHPWLNNLAEKAKRCNRRLKSQILLKKYLMKRRWKKNFIAVSAANRFKKISSSGALMALGV

SEQ ID NO: 79 - protein, 337 aa, Homo sapiens
MTNNSGSKAELVVGGKYKLVRKIGSGSFGDVYLGITTTNGEDVAVKLESQKVKHPQLLYESKLYTILQGGVGIPHMHWYGQEKDNNVLVMDLLGPSLEDLFNFCSRRFTMKTVLMLADQMISRIEYVHTKNFLHRDIKPDNFLMGTGRHCNKLFLIDFGLAKKYRDNRTRQHIPYREDKHLIGTVRYASINAHLGIEQSRRDDMESLGYVFMYFNRTSLPWQGLRAMTKKQKYEKISEKKMSTPVEVLCKGFPAEFAMYLNYCRGLRFEEVPDYMYLRQLFRILFRTLNHQYDYTFDWTMLKQKAAQQAASSSGQGQQAQTQTGKQTEKNKNNVKDN

SEQ ID NO: 80 - protein, 400 aa, Homo sapiens
MSGGGEQPDILSVGILVKERWKVLRKIGGGGFGEIYDALDMLTRENVALKVESAQQPKQVLKMEVAVLKKLQGGFVQIRIYIQGRHIALYFVAMSQFGRNLADLRRSQSRGTFTISTTLRLGRQILESIESIHSVGFLHRDIKPSNFAMGRFPSTCRKCYMLDFGLARQFTNSCGDVRPPRAVAGFRGTVRYASINAHRNREMGRHDDLWSLFYMLVEFVVGQLPWRKIKDKEQVGSIKERYDHRLMLKHLPPEFSIFLDHISSLDYFTKPDYQLLTSVFDNSIKTFGVIESDPFDWEKTGNDGSLTTTTTSTTPQLHTRLTPAAIGIANATPIPGDLLRENTDEVFPDEQLSDGENGIPVGVSPDKLPGSLGHPRPQEKDVWEEMDANKNKIKLGICKV

SEQ ID NO: 81 - protein, 591 aa, Homo sapiens
MEMYETLGKVGEGSYGTVMKCKHKNTGQIVAIKIFYERPEQSVNKIAMREIKFLKQFHHENLVNLIEVFRQKKKIHLVFEFIDHTVLDELQHYCHGLESKRLRKYLFQILRAIDYLHSNNIIHRDIKPENILVSQSGITKLCDFGFARTLAAPGDIYTDYVATRWYRAPELVLKDTSYGKPVDIWALGCMIIEMATGNPYLPSSSDLDLLHKIVLKVGNLSPHLQNIFSKSPIFAGVVLPQVQHPKNARKKYPKLNGLLADIVHACLQIDPADRISSSDLLHHEYFTRDGFIEKFMPELKAKLLQEAKVNSLIKPKESSKENELRKDERKTVYTNTLLSSSVLGKEIEKEKKPKEIKVRVIKVKGGRGDISEPKKKEYEGGLGQQDANENVHPMSPDTKLVTIEPPNPINPSTNCNGLKENPHCGGSVTMPPINLTNSNLMAANLSSNLFHPSVRLTERAKKRRTSSQSIGQVMPNSRQEDPGPIQSQMEKGIFNERTGHSDQMANENKRKLNFSRSDRKEFHFPELPVTIQSKDTKGMEVKQIKMLKRESKKTESSKIPTLLNVDQNQEKQEGGDGHCEGKNLKRNRFFF

SEQ ID NO: 82 - protein, 360 aa, Homo sapiens
MEKYEKLAKTGEGSYGVVFKCRNKTSGQVVAVKKFVESEDDPVVKKIALREIRMLKQLKHPNLVNLIEVFRRKRKMHLVFEYCDHTLLNELERNPNGVADGVIKSVLWQTLQALNFCHIHNCIHRDIKPENILITKQGIIKICDFGFAQILSNSHVGRIDLPDLIDAFAVPGDAYTDYVATRWYRAPELLVGDTQYGSSVDIWAIGCVFAELLTGQPLWPGKSDVDQLYLIIRTLGKLIPRHQSIFKSNGFFHGISIPEPEDMETLEEKFSDVHPVALNFMKGCLKMNPDDRLTCSQLLESSYFDSFQEAQIKRKARNEGRNRRRQQNQLLPLIPGSHISPTPDGRKQVLQLKFDHLPNI

SEQ ID NO: 83 - protein, 558 aa, Homo sapiens
MCTVVDPRIVRRYLLRRQLGQGAYGIVWKAVDRRTGEVVAIKKIFDAFRDKTDAQDMGFLLAPPTHTPVFLSLQRTFREITLLQEFGDHPNIISLLDVIRAENDRDIYLVFEFMDTDLNAVIRKGGLLQDVHVRSIFYQLLRATRFLHSGHVVHRDQKPSNVLLDANCTVKLCDFGLARSLGDLPEGPEDQAVTEYVATRWYRAPEVLLSSHRYTLGVDMWSLGCILGEMLRGRPLFPGTSTLHQLELILETIPPPSEEDLLALGSGCRASVLHQLGSRPRQTLDALLPPDTSPEALDLLRRLLVFAPDKRLSATQALQHPYVQRFHCPSDEWAREADVRPRAHEGVQLSVPEYRSRVYQMILECGGSSGTSREKGPEGVSPSQAHLHKPRADPQLPSRTPVQGPRPRPQSSPGHDPAEHAKNVPRQNSAPLLQTALLGNGERPPGAKEAPPLTLSLVKPSGRGAAPSLTSQAAAQVANQALIRGDWNRGGGVRVASVQQVPPRLPPEARPGRRMFSTSALQGAQGGARALLGGYSQAYGTVCHSALGHLPLLEGHHV

SEQ ID NO: 84 - protein, 751 aa, Homo sapiens
MAAILGDTIMVAKGLVKLTQAAVETHLQHLGIGGELIMAARALQSTAVEQIGMFLGKVQGQDKHEEYFAENFGGPEGEFHFSVPHAAGASTDFSSASAPDQSAPPSLGHAHSEGPAPAYVASGPFREAGFPGQASSPLGRANGRLFANPRDSFSAMGFQRRFFHQDQSPVGGLTAEDIEKARQAKARPENKQHKQTLSEHARERKVPVTRIGRLANFGGLAVGLGFGALAEVAKKSLRSEDPSGKKAVLGSSPFLSEANAERIVRTLCKVRGAALKLGQMLSIQDDAFINPHPASALLTPARQPVRQGLPRLLGTWRVHCCPGLAVGLGFGALAEVAKKSLRSEDPSGKKAVLGSSPFLSEANAERIVRTLCKVRGAALKLGQMLSIQDDAFINPHLAKIFERVRQSADFMPLKQMMKTLNNDLGPNWRDKLEYFEERPFAAASIGQVHLARMKGGREVAMKIQYPGVAQSINSDVNNLMAVLNMSNMLPEGLFPEHLIDVLRRELALECDYQREAACARKFRDLLKGHPFFYVPEIVDELCSPHVLTTELVSGFPLDQAEGLSQEIRNEICYNILVLCLRELFEFHFMQTDPNWSNFFYDPQQHKVALLDFGATREYDRSFTDLYIQIIRAAADRDRETVRAKSIEMKFLTGYEVKVMEDAHLDAILILGEAFASDEPFDFGTQSTTEKIHNLIPVMLRHRLVPPPEETYSLHRKMGGSFLICSKLKARFPCKAMFEEAYSNYCKRQAQQ

SEQ ID NO: 85 - protein, 626 aa, Homo sapiens
MVAPWRVSVRVCLSHLRCFELRQGLSLLRPSECPRDARLCWLLLGTLPKVVSLCGDVGEGAPDVLSRRRVRCSGAAGAGPAESLPRAGPLGGVFLHLRLWLRAGALLVKFFPLLLLYPLTYLAPSVSTLWLHLLLKATETSGPTYIKLGQWASTRRDLFSEAFCAQFSKLHVRVTPHPWTHTERFLRQAFGDDWGSILSFENREPVGSGCVAQVYKAYANTAFLETDSVQRLGRASCLPPFSHTGAVGGLRELFGYLGNGRKPPENLADQSFLERLLLPKADLVGSNAGVSRAQVPGHQPEATNLISVAVKVLHPGLLAQVHMDLLLMKIGSRVLGVLPGIKWLSLPEIVEEFEKLMVQQIDLRYEAQNLEHFQVNFRNVKAVKFPTPLRPFVTREVLVETYEESVPVSSYQQAGIPVDLKRKIARLGINMLLKMIFVDNFVHADLHPGNILVQGANGLSSSQEAQLQQADICDTLVVAVPSSLCPLRLVLLDAGIVAELQAPDLRNFRAVFMAVVMGQGQRVAELILHHARASECRDVEGFKTEMAMLVTQARKNTITLEKLHVSSLLSSVFKLLMTHKVKLESNFASIVFAIMVLEGLGRSLDPKLDILEAARPFLLTGPVCPP

SEQ ID NO: 86 - protein, 519 aa, Homo sapiens [sequence omitted]

SEQ ID NO: 87 - protein, 798 aa, Homo sapiens
MAASLPGPGSRLFRTYGAADGRRQRRPGREAAQWFPPQDRRRFFNSSGSSDASIGDPSQSDDPDDPDDPDFPGSPVRRRRRCPGGRVPKDRPSLTVTPKRWKLRARPSLTVTPRRLGLRARPPQKCSTPCGPLRLPPFPSRDSGRLSPDLSVCGQPRDGDELGISASLFSSLASPCPGSPTPRDSVISIGTSACLVAASAVPSDLHLPEVSLDRASLPCSQEEATGGAKDTRMVHQTRASLRSVLFGLMNSGTPEDSEFRADGKNMRESCCKRKLVVGNGPEGPGLSSTGKRRATGQDSCQERGLQEAVRREHQEASVPKGRIVPRGTDRLERTRSSRKSKHQEATETSLLHSHRFKKGQKLGKDSFPTQDLTPLQNACFWTKTRASFSFHKKKIVTDVSEVCSIYTTATSLSGSLLSECSNRPVMNRTSGAPSSWHSSSMYLLSPLNTLSISNKKASDAEKVYGECSQKGPVPFSHCLPTEKLQRCEKIGEGVFGEVFQTIADHTPVAIKIIAIEGPDLVNGSHQKTFEEILPEIIISKELSLLSGEVCNRTEGFIGLNSVHCVQGSYPPLLLKAWDHYNSTKGSANDRPDFFKDDQLFIVLEFEFGGIDLEQMRTKLSSLATAKSILHQLTASLAVAEASLRFEHRDLHWGNVLLKKTSLKKLHYTLNGKSSTIPSCGLQVSIIDYTLSRLERDGIVVFCDVSMDEDLFTGDGDYQFDIYRLMKKENNNRWGEYHPYSNVLWLHYLTDKMLKQMTFKTKCNTPAMKQIKRKIQEFHRTMLNFSSATDLLCQHSLFK

SEQ ID NO: 88 - protein, 1513 aa, Homo sapiens [sequence omitted]

SEQ ID NO: 89 - protein, 355 aa, Homo sapiens
SETDCYDIIEVLGKGTFGEVAKGWRRSTGEMVAIKILKNDAYRNRIIKNELKLLHCMRGLDPEEAHVIRFLEFFHDALKFYLVFELLEQNLFEFQKENNFAPLPARHIRTVTLQVLTALARLKELAIIHADLKPENIMLVDQTRCPFRVKVIDFGSASIFSEVRYVKEPYIQSRFYRAPEILLGLPFCEKVDVWSLGCVMAELHLGWPLYPGNNEYDQVRYICETQGLPKPHLLHAACKAHHFFKRNPHPDAANPWQLKSSADYLAETKVRPLERRKYMLKSLDQIETVNGGSVASRLTFPDREALAEHADLKSMVELIKRMLTWESHERISPSAALRHPFVSMQQLRSAHETTH

SEQ ID NO: 90 - protein, 703 aa, Homo sapiens
MNTPLQSAHTHLTLVEFIAYLVSDSPFPAASTLSLPTCNFDPCPTKLGAGTMPPAAAGGQRRPHAAPPAAYPHRNKVRAAEARTSRAVRREYEAGDEASGRRGRGGRMKSVQNARADRGMWVRERRAVVRGQENTVVVFAAAASPESDFLQIVLRDIAREMVPEYKSDLQPRTQMDAKKPRKCDLTPFLVLKARKKQKFTSAKHLWVQRKGKGVEDHADWQGQIWVGVEGKSAEGQGLNLTGAFNRSPENKVSPQGPALNALEIDSSYLDPQALALPQGWQGLLFPPLLGTELVGSKLSVQIQKPPSNIKNSRMTQVFHKNTSVTSLPFVDTKGKKNTVSFPHISKKVLLKSSLLYQVSADGLDPEKAGERPGFSPKPVLERPRIVGKSTVAAEEENQAHNQMPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKGHLPSRLRSAVTTTSMILNKVLRFSEPKLPHLQFEKVVSKY

SEQ ID NO: 91 - protein, 419 aa, Homo sapiens
MAGSGCAWGAEPPRFLEAFGRLWQVQSRLGSGSSASVYRVRCCGNPGSPPGALKQFLPPGTTGAAASAAEYGFRKERAALEQLQGHRNIVTLYGVFTIHFSPNVPSRCLLLELLDVSVSELLLYSSHQGCSMWMIQHCARDVLEALAFLHHEGYVHADLKPRNILWSAENECFKLIDFGLSFKEGNQDVKYIQTDGYRAPEAELQNCLAQAGLQSDTECTSAVDLWSLGIILLEMFSGMKLKHTVRSQEWKANSSAIIDHIFASKAVVNAAIPAYHLRDLIKSMLHDDPSRRIPAEMALCSPFFSIPFAPHIEDLVMLPTPVLRLLNVLDDDYLENEEEYEDVVEDVKEECQKYGPVVSLLVPKGNPGRGQVFVEYANAGDSKAAQKLLTGRMFDGKFVVATFYPLSAYKRGYLYQTLL

SEQ ID NO: 92 - protein, 661 aa, Homo sapiens
MKKFSRMPKSEGGSGGGAAGGGAGGAGAGAGCGSGGSSVGVRVFAVGRHQVTLEESLAEGGFSTVFLVRTHGGIRCALKRMYVNNMPDLNVCKREITIMKELSGHKNIVGYLDCAVNSISDNVWEVLILMEYCRAGQVVNQMNKKLQTGFTEPEVLQIFCDTCEAVARLHQCKTPIIHRDLKVENILLNDGGNYVLCDFGSATNKFLNPQKDGVNVVEEEIKKYTTLSYRAPEMINLYGGKPITTKADIWALGCLLYKLCFFTLPFGESQVAICDGNFTIPDNSRYSRNIHCLIRFMLEPDPEHRPDIFQVSYFAFKFAKKDCPVSNINKCCKQLLRHGALLTEILLFLQLFLNRMTASEAAARKSQIKARITDTIGPTETSIAPRQRPKANSATTATPSVLTIQSSATPVKVLAPGEFGNHRPKGALRPGNGPEILLGQGPPQQPPQQHRVLQQLQQGDWRLQQLHLQHRHPHQQQQQQQQQQQHHHHHHHHLLQDAYMQQYQHATQQQQMLQQQFLMHSVYQPQPSASQYPTMMPQYQQAFFQQQMLAQHQPSQQQASPEYLTSPQEFSPALVSYTSSLPAQVGTIMDSSYSANRSVADKEAIANFTNQKNISNPPDMSGWNPFGEDNFSKLTEEELLDREFDLLRSSKGHLKAYFASQ

SEQ ID NO: 93 - protein, 1615 aa, Homo sapiens
MFPLIGKTIIFDNFPDPSDTWEITETIGKGTYGKVFKVLNKKNGQKAAVKILDPIHDIDEEIEAEYNILKALSDHPNVVRFYGIYFKKDKVNGDKLWLVLELCSGGSVTDLVKGFLKRGERMSEPLIAYILHEALMGLQHLHNNKTIHRDVKGNNILLTTEGGVKLVDFGVSAQLTSTRHRRNTSVGTPFWMAPEVIACEQQLDTTYDARCDTWSLGITAIELGDGDPPLADLHPMRALFKIPRNPPPKLRQPELWSAEFNDFISKCLTKDYEKRPTVSELLQHKFITQIEGKDVMLQKQLTEFIGIHQCMGGTEKARRERIHTKKGNFNRPLISNLKDVDDLATLEILDENTVSEQLEKCYSRDQIYVYVGDILIALNPFQSLGLYSTKHSKLYIGSKRTASPPHIFAMADLGYQSIITYNSDQCIVISGESGAGKTENAHLLVQQLTVLGKANNRTLQEKILQVNNLVEAFGNACTIINDNSSRFGKYLEMKFTSSGAVVGAQISEYLLEKSRVIHQAIGEKNFHIFYYIYAGLAEKKKLAHYKLPENKPPRYLQNDHLRTVQDIMNNSFYKSQYELIEQCFKVIGFTMEQLGSIYSILAAILNVGNIEFSSVATEHQIDKSHISNHTALENCVSLLCIRADELQEALTSHCVVTRGETIIRPNTVEKATDVRDAMAKTLYGRLFSWIVNCINSLLKHDSSPSGNGDELSIGILDIFGFENFKKNSFEQLCINIANEQIQYYYNQHVFAWEQNEYLNEDVDARVIEYEDNWPLLDMFLQKPMGLLSLLDEESRFPKATDQTLVEKFEGNLKSQYFWRPKRMELSFGIHHYAGKVLYNASGFLAKNKTLVPTDIVLLLRSSDNSVIRQLVNHPLTKTGNLPHSKLKMLNYQMRTSEKLINLAKGDTGEATRHARETTNMKTQTVASYFRYSLMDLLSKMVVGQPHFVRCIKPNSERQARKYDKEKVLLQLRYTGILETARIRRLGFSHRILFANFIKRYYLLCYKSSEEPRMSPDTCATILEKAGLDNWALGKTKVFLKYYHVEQLNLMRKEAIDKLILIQACVRAFLCSRRYQKIQEKRKESAIIIQSAARGHLVGKQRKEIVDMKNTAVTTIQTSDQEFDYKKNFENTRESFVKKQAENAISANERFISAPNNKGSVSVVKTSTFKPEEETTNAVESNNRVYQTPKKMNNVYEEEVKQEFYLVGPEVSPKQKFVKDLEENSNLRKVEKEEAMIQSYYQRYTEERNCEESKAAYLERKAISERPSYPVPWLAENETSFKKTLEPTLSQRSIYQNANSMEKEKKTSVVTQSAPICSQEEGRGRLRHETVKERQVEPVTQAQEEEDKAAVFIQSKYRGYKRRQQLRKDKMSSFKHQKIVTTPTEVARNTHNLYSYPTKHEEINNIKKKDNKDSKATSEREACGLAIFSKQISKLSEEYFILQKKLNEMILSQQLKSLYLGVSHHKPINRRVSSQQCLSGVCKGEEPKILRPPRRPRKPKTLNNPEDSTYYYLLHKSIQEEKRRPRKDSQGKLLDLEDFYYKEFLPSRSGPKEHSPSLRERRPQQELQNQCIKANERCWAAESPEKEEEREPAANPYDFRRLLRKTSQRRRLVQQS

SEQ ID NO: 94 - protein, 68 aa, Homo sapiens
FKVGNLPGKGSFAGVCKVESIHTGLEISVQMIVKKAVRIQRVHNEMKMHCQLKHPPTLELYKYFKDKD

SEQ ID NO: 95 - protein, 945 aa, Homo sapiens
MTKSEEQQPLSLQKALQQCELVQNMIDLSISNLEGLRTKCATSNDLTQKEIRTLESKLVKYFSRQLSCKKKVALQERNAELDGFPQLRHWFRIVDVRKEVLEEISPGQLSLEDLLEMTDEQVCETVEKYGANREECARLNASLSCLRNVHMSGGNLSKQDWTIQWPTTETGKENNPVCPPEPTPWIRTHLSQSPRVPSKCVQHYCHTSPTPGAPVYTHVDRLTVDAYPGLCPPPPLESGHRSLPPSPRQRHAVRTPPRTPNIVTTVTPPGTPPMRKKNKLKPPGTPPPSSRKLIHLIPGFTALHRSKSHEFQLGHRVDEAHTPKAKKKSKPLNLKIHSSVGSCENIPSQQRSPLLSERSLRSFFVGHAPFLPSTPPVHTEANFSANTLSVPRWSPQIPRRDLGNSIKHRFSTKYWMSQTCTVCGKGMLFGLKCKNCKLKCHNKCTKEAPPCHLLIIHRGDPARLVRTESVPCDINNPLRKPPRYSDLHISQTLPKTNKINKDHIPVPYQPDSSSNPSSTTSSTPSSPAPPLPPSATPPSPLHPSPQCTRQQKNFNLPASHYYKYKQQFIFPDVVPVPETPTRAPQVILHPVTSNPILEGNPLLQIEVEPTSENEEVHDEAEESEDDFEEMNLSLLSARSFPRKASQTSIFLQEWDIPFEQLEIGELIGKGRFGQVYHGRWHGEVAIRLIDIERDNEDQLKAFKREVMAYRQTRHENVVLFMGACMSPPHLAIITSLCKGRTLYSVVRDAKIVLDVNKTRQIAQEIVKGMGYLHAKGILHKDLKSKNVFYDNGKVVITDFGLFSISGVLQAGRREDKLRIQNGWLCHLAPEIIRQLSPDTEEDKLPFSKHSDVFALGTIWYELHAREWPFKTQPAEAIIWQMGTGMKPNLSQIGMGKEISDILLFCWAFEQEERPTFTKLMDMLEKLPKRNRRLSHPGHFWKSAEL

SEQ ID NO: 96 - protein, 832 aa, Homo sapiens [sequence omitted]

SEQ ID NO: 97 - protein, 367 aa, Homo sapiens
MDDAAVLKRRGYLLGINLGEGSYAKVKSAYSERLKFNVAIKIIDRKKAPADFLEKFLPREIEILAMLNHCSIIKTYEIFETSHGKVYIVMELAVQGDLLELIKTRGALHEDEARKKFHQLSLAIKYCHDLDVVHRDLKCDNLLLDKDFNIKLSDFSFSKRCLRDDSGRMALSKTFCGSPAYAAPEVLQGIPYQPKVYDIWSLGVILYIMVCGSMPYDDSNIKKMLRIQKEHRVNFPRSKHLTGECKDLIYHMLQPDVNRRLHIDEILSHCWMQPKARGSPSVAINKEGESSRGTEPLWTPEPGSDKKSATKLEPEGEAQPQAQPETKPEGTAMQMSRQSEILGFPSKPSTMETEEGPPQQPPETRAQ

SEQ ID NO: 98 - protein, 31 aa, Homo sapiens
PREAVSIVSSCWVRPPATRLRALKILTHPAF

SEQ ID NO: 99 - protein, 350 aa, Homo sapiens
MEKQPQNSRRGLAPREVPPAVGLLLIMALMNTLLYLCLDHFFIAPRQSTVDPTHCPYGHFRIGQMKNCSPWLSCEELRTEVRQLKRVGEGAVKRVFLSEWKEHKVALSQLTSLEMKDDFLHGLQMLKSLQGTHVVTLLGYCEDDNTMLTEYHPLGSLSNLEETLNLSKYQNVNTWQHRLELAMDYVSIINYLHHSPVGTRVMCDSNDLPKTLSQYLLTSNFSILANDLDALPLVNHSSGMLVKCGHRELHGDFVAPEQLWPYGEDVPFHDDLMPSYDEKIDIWKIPDISSFLLGHIEGSDMVRFHLFDIHKACKSQTPSERPTAQDVLETYQKVLDTLRDAMMSQAREML

SEQ ID NO: 100 - protein, 472 aa, Homo sapiens
MDNIDEILEKTESSVCKELEIALVDQGDADKEIISNTYSQVLQKIHSEERLIATVQAKYKDSIEFKKQLIEYLKKIPSVDHLLSIKKTLKSLKALLRWKLVEKSNLEESDDPDGSQIEKIKEEITQLRNNVFQEIYHEREEYEMLTSLAQKWFPELPLLHPEIGLLKYMNSGGLLTMSLERDLLDAEPMKELSSKRPLVRSEVNGQIILLKGYSVDVDTEAKVIERAATYHRAWREAEGDSGLLPLIFLFLCKSDPMAYLMVPYYPRANLNAVQANMPLNSEETLKVMKGVAQGLHTLHKADIIHGSLHQNNVFALNREQGIVGDFDFTKSVSQRASVNMMVGDLSLMSPELKMGKPASPGSDLYAYGCLLLWLSVQNQEFEINKDGIPKVDQFHLDDKVKSLLCSLICYRSSMTAEQVLNAECFLMPKEQSVPNPEKDTEYTLYKKEEEIKTENLDKCMEKTRNGEANFDC

SEQ ID NO: 101 - protein, 424 aa, Homo sapiens
MSVGCPEPEPPRSLTCCGPGTAPGPGAGVPLLTEDMQALTLRTLAASDVTKHYELVRELGKGTYGKVDLVVYKGTGTKMALKFVNKSKTKLKNFLREVSITNSLSSSPFIIKVFDVVFETEDCYVFAQEYAPAGDLFDIIPPQVGLPEDTVKRCVQQLGLALDFMHGRQLVHRDIKPENVLLFDRECRRVKLADFGMTRRVGCRVKRVSGTIPYTAPEVCQAGRADGLAVDTGVDVWAFGVLIFCVLTGNFPWEAASGADAFFEEFVRWQRGRLPGLPSQWRRFTEPALRMFQRLLALEPERRGPAKEVFRFLKHELTSELRRRPSHRARKPPGDRPPAAGPLRLEAPGPLKRTVLTESGGGSRPAPPAVGSVPLPVPVPVPVPVPVPVPEPGLAPQGPPGRTDGRADKSKGQVVLATAIEICV

SEQ ID NO: 102 - protein, 649 aa, Homo sapiens
MDKYDVIKAIGQGAFGKAYLAKGKSDSKHCVIKEINFEKMPIQEKEASKKEVILLEKMKHPNIVAFFNSFQENGRLFIVMEYCDGGDLMKRINRQRGVLFSEDQILGWFVQISLGLKHIHDRKILHRDIKAQNIFLSKNGMVAKLGDFGIARVLNNSMELARTCIGTPYYLSPEICQNKPYNNKTDIWSLGCVLYELCTLKHPFEGNNLQQLVLKICQAHFAPISPGFSRELHSLISQLFQVSPRDRPSINSILKRPFLENLIPKYLTPEVSFEVTVWILAEILGCRSLTRVFGFRCEPLHPACIMVFKNIPSCFLQIKMIERPKIAAVCGHYDYYYAQLDMLRRRAHKPSYHPIPQENTGVEDYGQETRHGPSPSQWPAEYLQRKFEAQQYKLKVEKQLGLRPSSAEPNYNQRQELRSNGEEPRFQELPFRKNEMKEQEYWKQLEEIRQQYHNDMKEIRKKMGREPEDIEKDLKQMRLQNTKESKNPEQKYKAKKGVKFEINLDKCISDENILQEEEAMDIPNETLTFEDGMKFKEYECVKEHGDYTDKAFEKLHCPEAGFSTQTVAAVGNRRQWDGGAPQTLLQMMAVADITSTCPTGPDNGQVIVIEGIPGNRKQWRHEAPGTLMSVLAAAHLTSSSFSADEEFGM

SEQ ID NO: 103 - protein, 645 aa, Homo sapiens
MLKFQEAAKCVSGSTAISTYPKTLIARRYVLQQKLGSGSFGTVYLVSDKKAKRGEELKVLKEISVGELNPNETVQANLEAQLLSKLDHPAIVKFHASFVEQDNFCIITEYCEGRDLDDKIQEYKQAGKIFPENQIIEWFIQLLLGVDYMHERRILHRDLKSKNVFLKNNLLKIGDFGVSRLLMGSCDLATTLTGTPHYMSPEALKHQGYDTKSDIWSLACILYEMCCMNHAFAGSNFLSIVLKIVEGDTPSLPERYPKELNAIMESMLNKNPSLRPSAIEILKIPYLDEQLQNLMCRYSEMTLEDKNLDCQKEAAHIINAMQKRIHLQTLRALSEVQKMTPRERMRLRKLQAADEKARKLKKIVEEKYEENSKRMQELRSRNFQQLSVDVLHEKTHLKGMEEKEEQPEGRLSCSPQDEDEERWQGREEESDEPTLENLPESQPIPSMDLHELESIVEDATSDLGYHEIPEDPLVAEEYYADAFDSYCVESDEEEEEIALERPEKEIRNEGSQPAYRTNQQDSDIEALARCLENVLGCTSLDTKTITTMAEDMSPGPPIFNSVMARTKMKRMRESAMQKLGTEVFEEVYNYLKRARHQNASEAEIRECLEKVVPQASDCFEVDQLLYFEEQLLITMGKEPTLQNHL

SEQ ID NO: 104 - protein, 446 aa, Homo sapiens
  MOD_RES (1)..(446): “Xaa” represents any, other or unknown amino acid
MPSRAENYEVLYTIGTGSCGRCQKIQRKSDGKILVWKELHYGSMTEAEKQMLVSEVNLLCELKNPNIVHYYDRIIDRTNTTLYIVMEYCEEGDLASVITKGTKERQYLDEEFVLRVTTQLTLALKXCHRRSDGDHTVVRRDLKPASVFLDGKQNVKLGDLGLARILNHDTSFAKTFVGTPYYMSPEQTNHMSYNEKPDIWSLGCLPYESRALMPPFTAFSQKELAGKIREGKFRRILYRYSDELNEIIMRMLKDYHRPSVEEILENPLIADLVAEEQRRNLERRGRQLGEPEKLPDSSPVLSELKLKEIQLEEQERALKAGEERLEQKEQELCVCERLAEDRLAIPENLLKNYSLLKEQKFLSLASSPELLNLPSSVIKKKVHFSGESKENVMRSENPESQLTSKSKCKDLKXXCFMLHAAQLRAQALSDIEKNYQLKSRQILGMR

SEQ ID NO: 105 - protein, 399 aa, Homo sapiens
  MOD_RES (381): any, other or unknown amino acid
MLARRKPMLPALTINPTIAEGPSPTSEGASEANLVDLQKKLEELELDEQQKRLEAFLTQKAKVGELKDDDFERTSELDAGNGGVVTKVQHRPSGLIMARKLIHLEIKPAIRNQIIREHQVLHECNSPYIVGFYGAFYCDREISICMEHMDGGSLDQGLKEAKRIPEDILGKVSIAVLRGLAYLREKHQIMHRNVKPSNILVNSRGEIKLCDFGVSGQLIDSMANSFVGTRSYMAPERLQGTHYSVQSVIWSMDLSLVELAIERYPIPPPDAKELEAIFGQPVVDREEGEPHSISSWPGSPGRPNSGYGMDSLPAMAIFELLDYIVKEPPPKLPNGVFTPDFQEFVNKCLIKNPTERADLKMLTNHAFIKRSEVKEADFACXLCKTLGLNQPGTPTRTAV

SEQ ID NO: 106 - protein, 278 aa, Homo sapiens
NSLKSEEPILWTKGEILGKGAYGTVYCGLTSQGQLIAVKQVALDTSNKLAAEKEYRKLQEEVDLLKALKHVNIVAYLGTCLQENTVSIFMEFVPGGSISSIINRFGPLPEMVFCKYTKQILQGVAYLHENCVVHRDIKGNNVMLMPTGIIKLIDFGCARRLAWAGLNGTHSDMLKSMHGTPYWMAPEVINESGYGRKSDIWSIGCTVFEMATGKPPLASMDRMAAMFYIGAHRGLMPPLPDHFSENAADFVRMCLTRDQHERPSALQLLKHSFLERSH

SEQ ID NO: 107 - protein, 28 aa, Homo sapiens
DLTPQNILLDKSVFVYLGVFGLAREFML

SEQ ID NO: 108 - protein, 348 aa, Homo sapiens
RDCPPVIAPPPDHMKSIYTRAVIDPVLAPVGDSNVDGGAKSLDKQKKKTKMTDEEIMEKLRTIVSIGPVRKKYTRYEKIGQGASGTVFTATDVALGQKVAIKQINLQKQPKKELIINEILVMKELKNPNIVNFLDSYLVGDELFVVVEYLARGSLTDVVTETCMDEAQIAAVCRESLQALEFLHANQVIHRDIKSDSVLLGMEGSVKLTDFGFCAQITPEQSKRSTVVRTPHWMAPEVVTRKAYGPKVSVWSLGIMATEMVEGEPPYLNENPLRALCLIATNGIPELQNPETLSPIFRDFLNRCLETDVEKRGSAKELLQHLFLKLAKLLSSLTPLIMAAKEAMKSNR

SEQ ID NO: 109 - protein, 1106 aa, Homo sapiens
MMLGLESLPDPTDTWEIIETIGKGTYGKVYKVTNKRDGSLAAVKILDPVSDMDEEIEAEYNILQFLPNHPNVVKFYGMFYKADHCVGGQLWLVLELCNGGSVTELVKGLLRCGQRLDEAMISYILYGALLGLQHLHNNRIIHRDVKGNNILLTTEGGVKLVDFGVSAQLTSTRLRRNTSVGTPFWMAPEVIACEQQYDSSYDARCDVWSLGITAIELGDGDPPLFDMHPVKTLFKIPRNPPPTLLHPEKWCEEFNHFISQCLIKDFERRPSVTHLLDHPFIKGVHGKVLFLQKQLAKVLQDQKHQNPVAKTRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENIWQVPRRAEGGGMNVGILDIFGFENFQRNSFEQLCINIANEQIQYYFNQHVFALEQMEYQNEGIDAIPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGTWEPSDSPALKAFARPGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKKVREKREKGAIAIQS

SEQ ID NO: 110 - protein, 1009 aa, Homo sapiens
METCAGPHPLRLFLCRMQLCLALLLGPWRPGTAEEVILLDSKASQAELGWTALPSNGWEEISGVDEHDRPIRTYQVCNVLEPNQDNWLQTGWISRGRGQRIFVELQFTLRDCSSIPGAAGTCKETFNVYYLETEADLGRGRPRLGGSRPRKIDTIAADESFTQGDLGERKMKLNTEVREIGPLSRRGFHLAFQDVGACVALVSVRVYYKQCRATVRGLATFPATAAESAFSTLVEVAGTCVAHSEGEPGSPPRMHCGADGEWLVPVGRCSCSAGFQERGDICEACPPGFYKVSPRRRVCSPCPEHSRALENASTFCVCQDSYARSPTDPPSASCTRGPPSAPRDLQYSLSRSPLVLRLRWLPPADSGGRSDVTYSLLCLRCGREGPAGACEPCGPRVAFLPRQAGLRERAATLLHLRPGARYTVRVAVLNGVSGPAAALVPVGAVSINPGTVGPVPVAGVIRDRVEPQSVSLSWREPIPAGAPGANDTEYEIRYYEKVQSEQTYSMVKTGAPTVTVTNLKPATRYVFQIRAASPGPSWEAQSFNPSIEVQTLGEAASGSRDQSPAIVVTVVTISALLVLGSVMSVLAIWRRPCSYGKGGGDAHDEEELYFHFKVPTRRTFLDPQSCGDLLQAVHLFAKELDAKSVTLERSLGGGRFGELCCGCLQLPGRQELLVAVHMLRDSASDSQRLGFLAEALTLGQFDHSHIVRLEGVVTRGSTLMIVTEYMSHGALDGFLRRHEGQLVAGQLMGLLPGLASAMKYLSEMGYVHRGLAARHVLVSSDLVCKISGFGRGPRDRSEAVYTTMSGRSPALWAAPETLQFGHFSSASDVWSFGIIMWEVMAFGERPYWDMSGQDVIKAVEDGFRLPPPRNCPNLLHRLMLDCWQKDPGERPRFSQIHSILSKMVQDPEPPKCALTTCPRPPTPLADRAFSTFPSFGSVGAWLEALDLCRYKDSFAAAGYGSLEAVAEMTAQRDLVSLGISLAEHREALLSGISALQARVLQLQGQGVQV

SEQ ID NO: 111 - protein, 909 aa, Homo sapiens
MQPLDFSSGGSDPNISLSEKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQDVSSIFGLYIRDILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRGCILLGQVVDHIDSLMEEWFPGLLEIDICGEGETLLKKWALYSFNDGEEHQKILLDDLMKKAEEGDLLVNPDQPRLTIPISQIAPDLILADPPRNIMLNNDELEFEQAPEFLLDCFVCIHLYPSSDYISRHYMRTINIVQTGFAKCRWRVTVHGADHGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLR

SEQ ID NO: 112 - protein, 1164 aa, Homo sapiens
MAGAGGQHHPPGAAGGAAAGAGAAVTSAAASAGPGEDSSDSEAEQEGPQKLIRKVSTSGQIRTKTSIKEGQLLKQTSSFQRWKKRYFKLRGRTLYYAKDSKSLIFDEVDLSDASVAEASTKNANNSFTIITPFRRLMLCAENRKEMEDWISSLKSVQTREPYEVAQFNVEHFSGMHNWYACSHARPTFCNVCRESLSGVTSHGLSCEVCKFKAHKRCAVRATNNCKWTTLASIGKDIIEDEDGVAMPHQWLEGNLPVSAKCAVCDKTCGSVLRLQDWKCLWCKTMVHTACKDLYHPICPLGQCKVSIIPPIALNSTDSDGFCRATFSFCVSPLLVFVNSKSGDNQGVKFLRRFKQLLNPAQVFDLMNGGPHLGLRLFQKFDNFRILVCGGDGSVGWVLSEIDKLNLNKQCQLGVLPLGTGNDLARVLGWGGSYDDDTQLPQILEKLERASTKMLDRWSIMTYELKLPPKASLLPGPPEASEEFYMTIYEDSVATHLTKILNSDEHAVVISSAKTLCETVKDFVAKVEKTYDKTLENAVVADAVASKCSVLNEKLEQLLQALHTDSQAAPVLPGLSPLIVEEDAVESSSEESLGESKEQLGDDVTKPSSQKAVKPREIMLRANSLKKAVRQVIEEAGKVMDDPTVHPCEPANQSSDYDSTETDESKEEAKDDGAKESITVKTAPRSPDARASYGHSQTDSVPGPAVAASKENLPVLNTRIICPGLRAGLAASIAGSSIINKMLLANIDPFGATPFIDPDLDSVDGYSEKCVMNNYFGIGLDAKISLEFNNKREEHPEKCRSRTKNLMWYGVLGTRELLQRSYKNLEQRVQLECDGQYIPLPSLQGIAVLNIPSYAGGTNFWGGTKEDDIFAAPSFDDKILEVVAIFDSMQMAVSRVIKLQHHRIAQCRTVKITIFGDEGVPVQVDGEAWVQPPGIIKIVHKNRAQMLTRDRAFESTLKSWEDKQKCDSGKPVLRTHLYIHHAIDLATEEVSQMQLCSQAAEELITRICDAATIHCLLEQELAHAVNACSHALNKANPRCPESLTRDTATEIAINVKALYNETESLLVGRVPLQLESPHEERVSNALHSVEVELQKLTEIPWLYYILHPNEDEEPPMDCTKRNNRSTVFRIVPKFKKEKVQKQKTSSQPGSGDTESGSCEANSPGN

SEQ ID NO: 113 - protein, 884 aa, Homo sapiens
MALKPCLEAPIESGLCSGIECHAEHPTLVLMLFASVLVTCLEAAKLTVGFQTPWNISHPFSMQRLGAGLQIAMDKVNSELVDLGNFTTYTNSACSTKESLAIFNNQVQNEQISALFGPACPEAAEDIGDVLQESLQYLGWKHIGMFGGYSGASSWDGVDELWRVVENELKSHFIITASMRYTNNSLVLLQEHLWRISSIARVIILTCSSEDAKIILLAAANLGLNTGEFVFIILQQLEVGTVDHSMALVVRGPFLEGSTDKSENDAPPKGVWVTASHCPQLLQKRPWRRRLLETSLPNTEEVALPQHHLLGGAGGCQDLDPGWSGQRCGARTGQGQQVSPYSAYLHDAVLLYAETVKQVVKAGGDFQDGWQLVSALKGSSQTTVQGITGPVFVDAQGERHMDYSVYALQKSENGPLLLSFLHYDSYQSVTAVLLTLMILIPVLGAAIIGLILRMQRQNKDIWWQINFDDITILPQNKPSQRATPVSKGINSNSSSVMISVDLSSFVKSQQWEELFYAAVGLYQGNHVAIRYVGDQAEAWVRKPIVLQEIQLLAAYTFTTRSDKASDLGVSPGMLFLHRSPLGSHSNLKPSNCLMDGRLQYPHSMRTSHSFAELYWTAPELLQFPEMPWSGTPQGDVYSFAILMRELIYHWDHGPFDDLHEAPDEIINQIKDPAAAVPLQPSLPEEKGNEKIVAMVRVCWDESLEKRPSFSSIKKTLREASPRGHVSILASVMSKLEVYANYLEEVVQERTSQLTAEKRKVEKLLSTKVPSFTGEQLLAGRSVEPEHFESVTIFLSDIVGFTKLCSLSSPLQVVKLLNDVYSLFDHIITTYDVYKGKGEQTTFWLKDKEGFTLPLPNLLRKKPKSQRYCELISLQGDRSSYLTAS

SEQ ID NO: 114 - protein, 48 aa, Homo sapiens
RELDHPNICRFTGGCIALPDVVIVMEYCPKGSLMDVLLNDNISFNWGF

SEQ ID NO: 115 - protein, 7 aa, Artificial Sequence
  Description of Artificial Sequence Signature sequence
HGDXXXN

SEQ ID NO: 116 - protein, 5 aa, Artificial Sequence
  Description of Artificial Sequence Motif
HRDLK

SEQ ID NO: 117 - protein, 48 aa, Homo sapiens
TLAAPGDIYTDYVATRWYRAPELVLKDTSYGKPVDIWALGCMIIEMAT

SEQ ID NO: 118 - protein, 60 aa, Artificial Sequence
  Description of Artificial Sequence Synthetic peptide
TLAAPGDIYTDYVATRWYRAPELVLKDTSYGKYVYFGILSAFPRPVDIWALGCMIMRWPL

SEQ ID NO: 119 - protein, 19 aa, Artificial Sequence
  Description of Artificial Sequence Synthetic peptide
DMGFLLAPPTHTPVFLSLQ

SEQ ID NO: 120 - protein, 60 aa, Homo sapiens
RPPQKCSTPCGPLRLPPFPSRDSGRLSPDLSVCGQPRDGDELGISASLFSSLASPCPGSP

SEQ ID NO: 121 - protein, 59 aa, Artificial Sequence
  Description of Artificial Sequence Synthetic peptide
RPPQKCSTPAARSDFRPSPAAIRPLSPDLSVCGQPRDGDELGISASLFSSLASPCPGSP

SEQ ID NO: 122 - protein, 45 aa, Artificial Sequence
  Description of Artificial Sequence Synthetic peptide
HRLGECWQKMRRRQQGAAGGNFPVGGSFPEDVSPHQDSGYAPSPR

SEQ ID NO: 123 - protein, 72 aa, Homo sapiens
RPELPPVCRARPRACSHLIRLMQRCWQGDPRVRPTFQEITSETEDLCEKPDDEVKETAHDLDVKSPPEPRSE

SEQ ID NO: 124 - protein, 120 aa, Artificial Sequence
  Description of Artificial Sequence Synthetic peptide
RPELPPVCRARPRACSHLIRLMQRCWQGDPRVRPTFQGNGLNGELIRQVLAALLPVTGRWRSPGEGFRLESEVIIRVTCPLSSPQEITSETEDLCEKPDDEVKETAHDLDVKSPPEPRSE

SEQ ID NO: 125 - protein, 105 aa, Artificial Sequence
  Description of Artificial Sequence Synthetic peptide
SLACILYEMCCMNHAFAGSNFLSIVLKIVEGDTPSLPERYPKELNAIMESMLNKNPSLRPSAIEILKIPYLDEQLQNLMCRYSEMTLEDKNLDCQKEAAHIINAM

SEQ ID NO: 126 - DNA, 69 bp, Artificial Sequence
  Description of Artificial Sequence insert sequence
ccctatgctgaacagagggactacaaaggcaaatcagatgcagttcctgataaagagctaatagtctgg

SEQ ID NO: 127 - protein, 23 aa, Artificial Sequence
  Description of Artificial Sequence Synthetic peptide
PYAEQRDYKGKSDAVPDKELIVW

SEQ ID NO: 128 - protein, 33 aa, Artificial Sequence
  Description of Artificial Sequence Synthetic peptide
KHQNPVAKTRPYAEQRDYKGKSDAVPDKELIVW

SEQ ID NO: 129 - DNA, 24 bp, Artificial Sequence
  Description of Artificial Sequence Synthetic primer
gacgtttatctgggcatcaccacc

SEQ ID NO: 130 - DNA, 24 bp, Artificial Sequence
  Description of Artificial Sequence Synthetic primer
tcggacagtgccaatgaggtgttt

SEQ ID NO: 131 - DNA, 24 bp, Artificial Sequence
  Description of Artificial Sequence Synthetic primer
gtgattgacttcggatccgccagc

SEQ ID NO: 132 - DNA, 27 bp, Artificial Sequence
  Description of Artificial Sequence Synthetic primer
cttttcccttaccttcgtctcggccag

SEQ ID NO: 133 - DNA, 24 bp, Artificial Sequence
  Description of Artificial Sequence Synthetic primer
ccgggacctgggagccggcttttc

SEQ ID NO: 134 - DNA, 24 bp, Artificial Sequence
  Description of Artificial Sequence Synthetic primer
gctcgggacggctgaggctgcaac

SEQ ID NO: 135 - DNA, 24 bp, Artificial Sequence
  Description of Artificial Sequence Synthetic primer
aagaagctggtgctggagaagctg

SEQ ID NO: 136 - DNA, 24 bp, Artificial Sequence
  Description of Artificial Sequence Synthetic primer
aaccacttctctgtctccctcctc

SEQ ID NO: 137 - DNA, 24 bp, Artificial Sequence
  Description of Artificial Sequence Synthetic primer
tccgacaccataactgagttacag

SEQ ID NO: 138 - DNA, 21 bp, Artificial Sequence
  Description of Artificial Sequence Synthetic primer
ctgctcctgggccaataaagc

SEQ ID NO: 139 - DNA, 24 bp, Artificial Sequence
  Description of Artificial Sequence Synthetic primer
cctgatgggtgtctcacctcctct

SEQ ID NO: 140 - DNA, 24 bp, Artificial Sequence
  Description of Artificial Sequence Synthetic primer
ggagatgatggcaggacagctggg

SEQ ID NO: 141 - DNA, 21 bp, Artificial Sequence
  Description of Artificial Sequence Synthetic primer
ttggagggctcagaagaggac

SEQ ID NO: 142 - DNA, 24 bp, Artificial Sequence
  Description of Artificial Sequence Synthetic primer
aacaagtccctcatctccaggtga

SEQ ID NO: 143 - DNA, 57 bp, Artificial Sequence
  Description of Artificial Sequence Synthetic primer
aagcagtggtaacaacgcagagtacttttttttttttttttttttttttttttttvn

SEQ ID NO: 144 - DNA, 30 bp, Artificial Sequence
  Description of Artificial Sequence Synthetic primer
aagcagtggtaacaacgcagagtacgcggg

SEQ ID NO: 145 - DNA, 30 bp, Artificial Sequence
  Description of Artificial Sequence Synthetic primer
aagtggcaacagagataacgcgtacgcggg

SEQ ID NO: 146 - DNA, 23 bp, Artificial Sequence
  Description of Artificial Sequence Synthetic primer
aagcagtggtaacaacgcagagt

SEQ ID NO: 147 - DNA, 23 bp, Artificial Sequence
  Description of Artificial Sequence Synthetic primer
aagtggcaacagagataacgcgt

SEQ ID NO: 148 - DNA, 23 bp, Artificial Sequence
  Description of Artificial Sequence Synthetic primer
aagcagtggtaacaacgcagagt

SEQ ID NO: 149 - DNA, 25 bp, Artificial Sequence
  Description of Artificial Sequence Synthetic primer
ggacatgatgacgctgagtgctcag

SEQ ID NO: 150 - DNA, 25 bp, Artificial Sequence
  Description of Artificial Sequence Synthetic primer
gtgctccctgatggcgatcacagcg

SEQ ID NO: 151 - DNA, 24 bp, Artificial Sequence
  Description of Artificial Sequence Synthetic primer
ctcctgtcttcttgacctcagggc

SEQ ID NO: 152 - DNA, 22 bp, Artificial Sequence
  Description of Artificial Sequence Synthetic primer
caggtccagaagcccctggagc

SEQ ID NO: 153 - DNA, 24 bp, Artificial Sequence
  Description of Artificial Sequence Synthetic primer
ccgaaaatctcttgtactacagtc

SEQ ID NO: 154 - DNA, 24 bp, Artificial Sequence
  Description of Artificial Sequence Synthetic primer
catagcctggagttccacaggcag

SEQ ID NO: 155 - DNA, 25 bp, Artificial Sequence
  Description of Artificial Sequence Synthetic primer
gagcctactagaaacggtgtggacc

SEQ ID NO: 156 - DNA, 25 bp, Artificial Sequence
  Description of Artificial Sequence Synthetic primer
cttcaccaaacccttctttcaccag

SEQ ID NO: 157 - DNA, 25 bp, Artificial Sequence
  Description of Artificial Sequence Synthetic primer
aaaggcccagaggggacagacgtgg

SEQ ID NO: 158 - DNA, 25 bp, Artificial Sequence
  Description of Artificial Sequence Synthetic primer
cctcaattgttgtatttgaactctc

SEQ ID NO: 159 - DNA, 25 bp, Artificial Sequence
  Description of Artificial Sequence Synthetic primer
gaaaaggtggccattaagatcctgg

SEQ ID NO: 160 - DNA, 25 bp, Artificial Sequence
  Description of Artificial Sequence Synthetic primer
catccattcatcattcatgatgcag

SEQ ID NO: 161 - DNA, 24 bp, Artificial Sequence
  Description of Artificial Sequence Synthetic primer
gaagaggaggaggcccagaccatg

SEQ ID NO: 162 - DNA, 24 bp, Artificial Sequence
  Description of Artificial Sequence Synthetic primer
ggaagagttctgtggccacatagg

SEQ ID NO: 163 - DNA, 25 bp, Artificial Sequence
  Description of Artificial Sequence Synthetic primer
cttctttgacaaagaacgtaacatc

SEQ ID NO: 164 - DNA, 24 bp, Artificial Sequence
  Description of Artificial Sequence Synthetic primer
cagtatatccttacagttctgctc

SEQ ID NO: 165 - DNA, 25 bp, Artificial Sequence
  Description of Artificial Sequence Synthetic primer
caacagcggctccaaagccgaactc

SEQ ID NO: 166 - DNA, 25 bp, Artificial Sequence
  Description of Artificial Sequence Synthetic primer
tgcctgtctgggtttgggcctgctg

SEQ ID NO: 167 - DNA, 24 bp, Artificial Sequence
  Description of Artificial Sequence Synthetic primer
gtcccgaggcacattcaccattag

SEQ ID NO: 168 - DNA, 24 bp, Artificial Sequence
  Description of Artificial Sequence Synthetic primer
gatgtctcgatgcaagaatcccac

SEQ ID NO: 169 - DNA, 25 bp, Artificial Sequence
  Description of Artificial Sequence Synthetic primer
gagatgtatgaaacccttggaaaag

SEQ ID NO: 170 - DNA, 25 bp, Artificial Sequence
  Description of Artificial Sequence Synthetic primer
catacagacactatgaacacgattc

SEQ ID NO: 171 - DNA, 25 bp, Artificial Sequence
  Description of Artificial Sequence Synthetic primer
gcctatggcattgtgtggaaggcag

SEQ ID NO: 172 - DNA, 25 bp, Artificial Sequence
  Description of Artificial Sequence Synthetic primer
cagcaggggcaggtggcccagtgcc

SEQ ID NO: 173 - DNA, 25 bp, Artificial Sequence
  Description of Artificial Sequence Synthetic primer
gtttcggtttcaccgcagcctcgcc

SEQ ID NO: 174 - DNA, 25 bp, Artificial Sequence
  Description of Artificial Sequence Synthetic primer
tggagaaggaaagaatgggagaaac

SEQ ID NO: 175 - DNA, 25 bp, Artificial Sequence
  Description of Artificial Sequence Synthetic primer
gaaaaggaggacatggagacccagg

SEQ ID NO: 176 - DNA, 25 bp, Artificial Sequence
  Description of Artificial Sequence Synthetic primer
ctttacgcagcttctcacgctttcg

SEQ ID NO: 177 - DNA, 25 bp, Artificial Sequence
  Description of Artificial Sequence Synthetic primer
cctaaagctttttaagaaccagctg

SEQ ID NO: 178 - DNA, 25 bp, Artificial Sequence
  Description of Artificial Sequence Synthetic primer
ctgatgaatccaagcatgcttgagg

SEQ ID NO: 179 - DNA, 25 bp, Artificial Sequence
  Description of Artificial Sequence Synthetic primer
gattttggtctggccaagtgcaacg

SEQ ID NO: 180 - DNA, 25 bp, Artificial Sequence
  Description of Artificial Sequence Synthetic primer
gtatacatcgtgcttggtgtcgaag

SEQ ID NO: 181 - DNA, 25 bp, Artificial Sequence
  Description of Artificial Sequence Synthetic primer
agggctcctatgcaaaagtaaaatc

SEQ ID NO: 182 - DNA, 22 bp, Artificial Sequence
  Description of Artificial Sequence Synthetic primer
ccagcagtggctgaggatctcg

SEQ ID NO: 183 - DNA, 25 bp, Artificial Sequence
  Description of Artificial Sequence Synthetic primer
ggaacgagatcttcttgatgctgag

SEQ ID NO: 184 - DNA, 25 bp, Artificial Sequence
  Description of Artificial Sequence Synthetic primer
ggaagggctgctgcgaagctttctg

SEQ ID NO: 185 - DNA, 25 bp, Artificial Sequence
  Description of Artificial Sequence Synthetic primer
gaatggcaggctgtttattgtaatg

SEQ ID NO: 186 - DNA, 25 bp, Artificial Sequence
  Description of Artificial Sequence Synthetic primer
gggaataagattctctaaaaagggc

SEQ ID NO: 187 - DNA, 25 bp, Artificial Sequence
  Description of Artificial Sequence Synthetic primer
cagctaagtgtgtgagtggatcaac

SEQ ID NO: 188 - DNA, 25 bp, Artificial Sequence
  Description of Artificial Sequence Synthetic primer
ccatcgtgatcagcaactgctcttc

SEQ ID NO: 189 - DNA, 25 bp, Artificial Sequence
  Description of Artificial Sequence Synthetic primer
ctgagaactatgaagtgttgtacac

SEQ ID NO: 190 - DNA, 25 bp, Artificial Sequence
  Description of Artificial Sequence Synthetic primer
ccaggatctgtctgctttttagttg

SEQ ID NO: 191 - DNA, 25 bp, Artificial Sequence
  Description of Artificial Sequence Synthetic primer
gccggcgctcaccatcaaccctacc

SEQ ID NO: 192 - DNA, 25 bp, Artificial Sequence
  Description of Artificial Sequence Synthetic primer
ctcactgagcatctttaggtccgcc

SEQ ID NO: 193 - DNA, 24 bp, Artificial Sequence
  Description of Artificial Sequence Synthetic primer
cctatcctatggaccaagggtgag

SEQ ID NO: 194 - DNA, 24 bp, Artificial Sequence
  Description of Artificial Sequence Synthetic primer
gtgctccgatgtaaaacatggcgg

SEQ ID NO: 195 - DNA, 23 bp, Artificial Sequence
  Description of Artificial Sequence Synthetic primer
gatgatgaagagactgccctccc

SEQ ID NO: 196 - DNA, 25 bp, Artificial Sequence
  Description of Artificial Sequence Synthetic primer
cattgcttctttagctgccatgatc

SEQ ID NO: 197 - DNA, 22 bp, Artificial Sequence
  Description of Artificial Sequence Synthetic primer
gatgccatgcagggccttcagc

SEQ ID NO: 198 - DNA, 24 bp, Artificial Sequence
  Description of Artificial Sequence Synthetic primer
caaagtcaacgagcttaactcctc

SEQ ID NO: 199 - DNA, 24 bp, Artificial Sequence
  Description of Artificial Sequence Synthetic oligonucleotide
ccgccacgccagctactcatctac

SEQ ID NO: 200 - DNA, 24 bp, Artificial Sequence
  Description of Artificial Sequence Synthetic oligonucleotide
tctcttcaacagggtctccactcg

SEQ ID NO: 201 - DNA, 18 bp, Artificial Sequence
  Description of Artificial Sequence Synthetic oligonucleotide
ttctcccagatccacagc

SEQ ID NO: 202 - DNA, 21 bp, Artificial Sequence
  Description of Artificial Sequence Synthetic oligonucleotide
accggagtcctgccttggaag

SEQ ID NO: 203 - DNA, 20 bp, Homo sapiens
gggttcgagagctgcagagr

SEQ ID NO: 204 - DNA, 19 bp, Homo sapiens
agagaccacagcagaagcy

SEQ ID NO: 205 - DNA, 16 bp, Homo sapiens
gctggaagagaccacr

SEQ ID NO: 206 - DNA, 18 bp, Homo sapiens
catgaagcctgagaatgk

SEQ ID NO: 207 - DNA, 21 bp, Homo sapiens
agaaaccagagcaacgcttar

SEQ ID NO: 208 - DNA, 25 bp, Homo sapiens
ccctccaaagactaagcaccccgam

SEQ ID NO: 209 - DNA, 27 bp, Homo sapiens
cacagtgaaagcagcagccacaagccm

SEQ ID NO: 210 - DNA, 17 bp, Homo sapiens
agccattgggaaaggcr

SEQ ID NO: 211 - DNA, 17 bp, Homo sapiens
ctggttcggcctggccs

SEQ ID NO: 212 - DNA, 14 bp, Homo sapiens
tgcaggacggggay

SEQ ID NO: 213 - DNA, 21 bp, Homo sapiens
aacgtgctggtcttcgacccr

SEQ ID NO: 214 - DNA, 13 bp, Homo sapiens
ccgcgtgctccty

SEQ ID NO: 215 - DNA, 19 bp, Homo sapiens
aaccttacagcaaagccgy

SEQ ID NO: 216 - DNA, 23 bp, Homo sapiens
atcagtgactttggattgtcaam

SEQ ID NO: 217 - DNA, 24 bp, Homo sapiens
tccagatcgtgcaactgctgtggs

SEQ ID NO: 218 - DNA, 20 bp, Homo sapiens
tggccgtgctggagccctay

SEQ ID NO: 219 - DNA, 14 bp, Homo sapiens
gtcgccctcccccr

SEQ ID NO: 220 - DNA, 16 bp, Homo sapiens
tggtgtttcctgaggr

SEQ ID NO: 221 - DNA, 19 bp, Homo sapiens
ggctcggctcgggtgcctk

SEQ ID NO: 222 - DNA, 15 bp, Homo sapiens
atggcttcgaccacm

SEQ ID NO: 223 - DNA, 17 bp, Homo sapiens
ccgggatggaaagtggs

SEQ ID NO: 224 - DNA, 29 bp, Homo sapiens
tgaatggaggtggcctggtgaacccccay

SEQ ID NO: 225 - DNA, 28 bp, Homo sapiens
gcttcagggccgagtacaaggccagtgs

SEQ ID NO: 226 - DNA, 19 bp, Homo sapiens
caccccccagccccggcgs

SEQ ID NO: 227 - DNA, 17 bp, Homo sapiens
aggtgtgccaggccccy

SEQ ID NO: 228 - DNA, 22 bp, Homo sapiens
sctctgtatttcaggaagctct

SEQ ID NO: 229 - DNA, 29 bp, Homo sapiens
agccccagtgagaacaatgctggtggggs

SEQ ID NO: 230 - DNA, 21 bp, Homo sapiens
ttttggatgattgcccgccam

SEQ ID NO: 231 - DNA, 22 bp, Homo sapiens
ctaagtagttcagttttgggar

SEQ ID NO: 232 - DNA, 22 bp, Homo sapiens
tccacattgcggaggttctgtr

SEQ ID NO: 233 - DNA, 23 bp, Homo sapiens
gcaaatgaaaatgttcatcctay

SEQ ID NO: 234 - DNA, 30 bp, Homo sapiens
atcctggttctgtgcctgagggagctgtty

SEQ ID NO: 235 - DNA, 15 bp, Homo sapiens
cccctgacgttctgr

SEQ ID NO: 236 - DNA, 18 bp, Homo sapiens
aggccaccaacctcatcy

SEQ ID NO: 237 - DNA, 15 bp, Homo sapiens
cctcctcacgggccy

SEQ ID NO: 238 - DNA, 22 bp, Homo sapiens
aaactaaatccacgtaagatcm

SEQ ID NO: 239 - DNA, 12 bp, Homo sapiens
agcacaccctgy

SEQ ID NO: 240 - DNA, 16 bp, Homo sapiens
cagccgtcccgagcgr

SEQ ID NO: 241 - DNA, 16 bp, Homo sapiens
ttgtgccaaggggaay

SEQ ID NO: 242 - DNA, 20 bp, Homo sapiens
gcccccaacccctgagccak

SEQ ID NO: 243 - DNA, 20 bp, Homo sapiens
caaaataatcagcaacattm

SEQ ID NO: 244 - DNA, 20 bp, Homo sapiens
acttcacgagcagtcaggcr

SEQ ID NO: 245 - DNA, 16 bp, Homo sapiens
ctggatgtcagtgttw

SEQ ID NO: 246 - DNA, 24 bp, Homo sapiens
attatcacgtggagcagttaaaty

SEQ ID NO: 247 - DNA, 25 bp, Homo sapiens
gattcaagcttgtgtcagagcatty

SEQ ID NO: 248 - DNA, 14 bp, Homo sapiens
tccagtggcccacs

SEQ ID NO: 249 - DNA, 17 bp, Homo sapiens
acagtggtcgaatggcm

SEQ ID NO: 250 - DNA, 15 bp, Homo sapiens
gtcatggagctcgcr

SEQ ID NO: 251 - DNA, 30 bp, Homo sapiens
ctgctgatcatggccctgatgaatactctr

SEQ ID NO: 252 - DNA, 17 bp, Homo sapiens
cctgaggtgtgccaggy

SEQ ID NO: 253 - DNA, 24 bp, Homo sapiens
tgatgcatttgattcctattgtgw

SEQ ID NO: 254 - DNA, 17 bp, Homo sapiens
aagttcatttcagtggr

SEQ ID NO: 255 - DNA, 18 bp, Homo sapiens
tactgtagtgcgtcgggr

SEQ ID NO: 256 - DNA, 19 bp, Homo sapiens
tggaggccatctttggccr

SEQ ID NO: 257 - DNA, 21 bp, Homo sapiens
ggggtgagcggccagctcatm

SEQ ID NO: 258 - DNA, 20 bp, Homo sapiens
acctggtggacctgcagaak

SEQ ID NO: 259 - DNA, 17 bp, Homo sapiens
gcgccccaacagcggty

SEQ ID NO: 260 - DNA, 14 bp, Homo sapiens
gggatggacagcck

SEQ ID NO: 261 - DNA, 23 bp, Homo sapiens
tggtccctgccggcgtgcctctr

SEQ ID NO: 262 - DNA, 17 bp, Homo sapiens
cagttttcactgctacy

SEQ ID NO: 263 - DNA, 22 bp, Homo sapiens
tttctgcaaaaacagctggccr

SEQ ID NO: 264 - DNA, 20 bp, Homo sapiens
gtggctgggcaactgatggr

SEQ ID NO: 265 - DNA, 12 bp, Homo sapiens
gtagccgtgcay

SEQ ID NO: 266 - DNA, 27 bp, Homo sapiens
ggttgtgccccatggcctatggttgar

SEQ ID NO: 267 - DNA, 20 bp, Homo sapiens
gacttgattttggctgaccy

SEQ ID NO: 268 - DNA, 28 bp, Homo sapiens
gggaggttcatatgacgatgacacccar

SEQ ID NO: 269 - DNA, 20 bp, Artificial Sequence
  Description of Artificial Sequence Synthetic primer
ggagctgtcgtattccagtc

SEQ ID NO: 270 - DNA, 21 bp, Artificial Sequence
  Description of Artificial Sequence Synthetic primer
aacccctcaagacccgtttag

SEQ ID NO: 271 - protein, 19 aa, Artificial Sequence
  Description of Artificial Sequence  Synthetic peptide
EEEYEEYEEEYEEEYEEEY

SEQ ID NO: 272 - DNA, 26 bp, Homo sapiens
ccgcgaggcagcagatcgccatgtcy

SEQ ID NO: 273 - DNA, 22 bp, Homo sapiens
ctcttcattgtgctggaatttk

SEQ ID NO: 274 - DNA, 31 bp, Homo sapiens
attcagaatatctctaaaacatggaaaattk

SEQ ID NO: 275 - DNA, 25 bp, Homo sapiens
gattcaagcttgtgtcagagcatty

SEQ ID NO: 276 - DNA, 21 bp, Homo sapiens
ggggtgagcggccagctcatm

SEQ ID NO: 277 - DNA, 18 bp, Artificial Sequence
  Description of Artificial Sequence Synthetic primer
gaatcttggatccagtaa

(19) United States
(12) Patent Application Publication
(10) Pub. No.: US 2006/0216730 A1
(43) Pub. Date: Sep. 28, 2006

(54) Novel human protein kinases and protein kinase-like enzymes

(75) Inventors: Gregory D. Plowman, San Carlos, CA (US); David Whyte, Belmont, CA (US); Gerard Manning, South San Francisco, CA (US); Sucha Sudarsanam, South San Francisco, CA (US); Ricardo Martinez, Foster City, CA (US); Peter Flanagan, San Francisco, CA (US); Douglas Clary, South San Francisco, CA (US)

Correspondence Address: FOLEY AND LARDNER LLP;SUITE 500, 3000 K STREET NW, WASHINGTON, DC, 20007, US

(73) Assignee: SUGEN, INC.

(21) Appl. No.: 11/320,072

(22) Filed: Dec. 29, 2005

Related U.S. Application Data
(62) Division of application No. 10/130,978, filed on Oct. 29, 2002, now abandoned.
(60) Provisional application No. 60/168,997, filed on Dec. 3, 1999.
(60) Provisional application No. 60/174,185, filed on Dec. 27, 1999.
(60) Provisional application No. 60/178,078, filed on Jan. 25, 2000.
(60) Provisional application No. 60/179,364, filed on Jan. 31, 2000.
(60) Provisional application No. 60/183,173, filed on Feb. 17, 2000.
(60) Provisional application No. 60/187,150, filed on Mar. 6, 2000.
(60) Provisional application No. 60/190,162, filed on Mar. 17, 2000.
(60) Provisional application No. 60/193,404, filed on Mar. 29, 2000.
(60) Provisional application No. 60/195,953, filed on Apr. 10, 2000.

(51) Int. Cl. C12Q 1/68; G01N 33/53; C07H 21/04; C12P 21/06; C12N 9/12

(52) U.S. Cl. 435/6; 435/69.1; 435/194; 435/320.1; 435/325; 536/23.2; 530/388.26; 435/7.1

(57) ABSTRACT

The present invention relates to kinase polypeptides, nucleotide sequences encoding the kinase polypeptides, as well as various products and methods useful for the diagnosis and treatment of various kinase-related diseases and conditions. Through the use of a bioinformatics strategy, mammalian members of the of PTK's and STK's have been identified and their protein structure predicted.

The present invention is a divisional application of U.S. application Ser. No. 10/130,978, filed Oct. 29, 2002, which is a U.S. national stage application of PCT/US2000/32085, filed Nov. 22, 2000, which claims priority on provisional application Ser. Nos. 60/190,162; 60/174,185; 60/168,997; 60/179,364; 60/183,173; 60/178,078; 60/193,404; 60/195,953; and 60/187,150, all of which are hereby incorporated by reference in their entirety.

### FIELD OF THE INVENTION

The present invention relates to kinase polypeptides, nucleotide sequences encoding the kinase polypeptides, as well as various products and methods useful for the diagnosis and treatment of various kinase-related diseases and conditions.

### BACKGROUND OF THE INVENTION

The following description of the background of the invention is provided to aid in understanding the invention, but is not admitted to be or to describe prior art to the invention.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function.

Protein phosphorylation plays a pivotal role in cellular signal transduction. Among the biological functions controlled by this type of postranslational modification are: cell division, differentiation and death (apoptosis); cell motility and cytoskeletal structure; control of DNA replication, transcription, splicing and translation; protein translocation events from the endoplasmic reticulum and Golgi apparatus to the membrane and extracellular space; protein nuclear import and export; regulation of metabolic reactions, etc. Abnormal protein phosphorylation is widely recognized to be causally linked to the etiology of many diseases including cancer as well as immunologic, neuronal and metabolic disorders.

The following abbreviations are used for kinases throught this application:

ASK Apoptosis signal-regulating kinase

CaMK Ca2+/calmodulin-dependent protein kinase

CCRK Cell cycle-related kinase

CDK Cyclin-dependent kinase

CK Casein kinase

DAPK Death-associated protein kinase

DM myotonic dystrophy kinase

Dyrk dual-specificity-tyrosine phosphorylating-regulated kinase

GAK Cyclin G-associated kinase

GRK G-protein coupled receptor

GuC Guanylate cyclase

HIPK Homeodomain-interacting protein kinase

IRAK Interleukin-1 receptor-associated kinase

MAPK Mitogen activated protein kinase

MAST Microtubule-associated STK

MLCK Myosin-light chain kinase

MLK Mixed lineage kinase

NIMA NimA-related protein kinase

PKA cAMP-dependent protein kinase

RSK Ribosomal protein S6 kinase

RTK Receptor tyrosine kinase

SGK Serum and glucocorticoid-regulated kinase

STK serine threonine kinase

ULK UNC-51-like kinase

The best-characterized protein kinases in eukaryotes phosphorylate proteins on the hydroxyl substituent of serine, threonine and tyrosine residues, which are the most common phospho-acceptor amino acid residues. However, phosphorylation on histidine has also been observed in bacteria.

The presence of a phosphate moiety modulates protein function in multiple ways. A common mechanism includes changes in the catalytic properties (Vmax and Km) of an enzyme, leading to its activation or inactivation.

A second widely recognized mechanism involves promoting protein-protein interactions. An example of this is the tyrosine autophosphorylation of the ligand-activated EGF receptor tyrosine kinase. This event triggers the high-affinity binding to the phosphotyrosine residue on the receptor's C-terminal intracellular domain to the SH2 motif of the adaptor molecule Grb2. Grb2, in turn, binds through its SH3 motif to a second adaptor molecule, such as SHC. The formation of this ternary complex activates the signaling events that are responsible for the biological effects of EGF. Serine and threonine phosphorylation events also have been recently recognized to exert their biological function through protein-protein interaction events that are mediated by the high-affinity binding of phosphoserine and phosphothreonine to WW motifs present in a large variety of proteins (Lu, P. J. et al (1999) Science 283:1325-1328).

A third important outcome of protein phosphorylation is changes in the subcellular localization of the substrate. As an example, nuclear import and export events in a large diversity of proteins are regulated by protein phosphorylation (Drier E. A. et al (1999) Genes Dev 13: 556-568).

Protein kinases are one of the largest families of eukaryotic proteins with several hundred known members. These proteins share a 250-300 amino acid domain that can be subdivided into 12 distinct subdomains that comprise the common catalytic core structure. These conserved protein motifs have recently been exploited using PCR-based and bioinformatic strategies leading to a significant expansion of the known kinases. Multiple alignment of the sequences in the catalytic domain of protein kinases and subsequent parsimony analysis permits their segregation into sub-families of related kinases.

Kinases largely fall into two groups: those specific for phosphorylating serines and threonines, and those specific for phosphorylating tyrosines. Some kinases, referred to as “dual specificity” kinases, are able to phosphorylate on tyrosine as well as serine/threonine residues.

Protein kinases can also be characterized by their location within the cell. Some kinases are transmembrane receptor-type proteins capable of directly altering their catalytic activity in response to the external environment such as the binding of a ligand. Others are non-receptor-type proteins lacking any transmembrane domain. They can be found in a variety of cellular compartments from the inner surface of the cell membrane to the nucleus.

Many kinases are involved in regulatory cascades wherein their substrates may include other kinases whose activities are regulated by their phosphorylation state. Ultimately the activity of some downstream effector is modulated by phosphorylation resulting from activation of such a pathway. The conserved protein motifs of these kinases have recently been exploited using PCR-based cloning strategies leading to a significant expansion of the known kinases.

Multiple alignment of the sequences in the catalytic domain of protein kinases and subsequent parsimony analysis permits the segregation of related kinases into distinct branches of subfamilies including: tyrosine kinases (PTK's), dual-specificity kinases, and serine/threonine kinases (STK's). The latter subfamily includes cyclic-nucleotide-dependent kinases, calcium/calmodulin kinases, cyclin-dependent kinases (CDK's), MAP-kinases, serine-threonine kinase receptors, and several other less defined subfamilies.

The protein kinases may be classified into several major groups including AGC, CAMK, Casein kinase 1, CMGC, STE, tyrosine kinases, and atypical kinases (Plowman, G D et al., Proceedings of the National Academy of Sciences, USA, Vol. 96, Issue 24, 13603-13610, Nov. 23, 1999; see also www.kinase.com). In addition, there are a number of minor yet distinct families, including families related to worm- or fungal-specific kinases, and a family designated “other” to represent several smaller families. Within each group are several distinct families of more closely related kinases. In addition, an “atypical” family represents those protein kinases whose catalytic domain has little or no primary sequence homology to conventional kinases, including the A6 kinases and P13 kinases.

AGC Group

The AGC kinases are basic amino acid-directed enzymes that phosphorylate residues found proximal to Arg and Lys. Examples of this group are the G protein-coupled receptor kinases (GRKs), the cyclic nucleotide-dependent kinases (PKA, PKC, PKG), NDR or DBF2 kinases, ribosomal S6 kinases, AKT kinases, myotonic dystrophy kinases (DMPKs), MAPK interacting kinases (MNKs), MAST kinases, and Mo3C11.1_ce family originally identified only in nematodes.

GRKs regulate signaling from heterotrimeric guanine protein coupled receptors (GPCRs). Mutations in GPCRs cause a number of human diseases, including retinitis pigmentosa, stationary night blindness, color blindness, hyperfunctioning thyroid adenomas, familial precocious puberty, familial hypocalciuric hypercalcemia and neonatal severe hyperparathroidism (OMIM, http://www.ncbi.nlm.nih.gov/Omim/). The regulation of GPCRs by GRKs indirectly implicates GRKs in these diseases.

The cAMP-dependent protein kinases (PKA) consist of heterotetramers comprised of 2 catalytic (C) and 2 regulatory (R) subunits, in which the R subunits bind to the second messenger cAMP, leading to dissociation of the active C subunits from the complex. Many of these kinases respond to second messengers such as cAMP resulting in a wide range of cellular responses to hormones and neurotransmitters.

AKT is a mammalian proto-oncoprotein regulated by phosphatidylinositol 3-kinase (PI3-K), which appears to function as a cell survival signal to protect cells from apoptosis. Insulin receptor, RAS, PI3-K, and PDK1 all act as upstream activators of AKT, whereas the lipid phosphatase PTEN functions as a negative regulator of the PI3-K/AKT pathway. Downstream targets for AKT-mediated cell survival include the pro-apoptotic factors BAD and Caspase9, and transcription factors in the forkhead family, such as DAF-16 in the worm. AKT is also an essential mediator in insulin signaling, in part due to its use of GSK-3 as another downstream target.

The S6 kinases regulate a wide array of cellular processes involved in mitogenic response including protein synthesis, translation of specific mRNA species, and cell cycle progression from G1 to S phase. The gene has been localized to chromosomal region 17q23 and is amplified in breast cancer (Couch, et al., Cancer Res. April 1999;59(7):1408-11).

CAMK Group

The CAMK kinases are also basic amino acid-directed kinases. They include the Ca2+/calmodulin-regulated and AMP-dependent protein kinases (AMPK), myosin light chain kinases (MLCK), MAP kinase activating protein kinases (MAPKAPKs) checkpoint 2 kinases (CHK2), death-associated protein kinases (DAPKs), phosphorylase kinase (PHK), Rac and Rho-binding Trio kinases, a “unique” family of CAMKs, and the EMK-related protein kinases.

The EMK family of STKs are involved in the control of cell polarity, microtubule stability and cancer. One member of the EMK family, C-TAK1, has been reported to control entry into mitosis by activating Cdc25C which in turn dephosphorylates Cdc2. Also included in the EMK family is MAKV, which has been shown to be overexpressed in metastatic tumors (Dokl. Akad. Nauk 354 (4), 554-556 (1997)).

CMGC Group

The CMGC kinases are “proline-directed” enzymes phosphorylating residues that exist in a proline-rich context. They include the cyclin-dependent kinases (CDKs), mitogen-activated protein kinases (MAPKs), GSK3s, RCKs, and CLKs. Most CMGC kinases have larger-than-average kinase domains owing to the presence of insertions within subdomains X and XI.

CDK's play a pivotal role in the regulation of mitosis during cell division. The process of cell division occurs in four stages: S phase, the period during which chromosomes duplicate, G2, mitosis and G1 or interphase. During mitosis the duplicated chromosomes are evenly segregated allowing each daughter cell to receive a complete copy of the genome. A key mitotic regulator in all eukaryotic cells is the STK cdc2, a CDK regulated by cyclin B. However some CDK-like kinases, such as CDK5 are not cyclin associated nor are they cell cycle regulated.

MAPKs play a pivotal role in many cellular signaling pathways, including stress response and mitogenesis (Lewis, T. S., Shapiro, P. S., and Ahn, N. G. (1998) Adv. Cancer Res. 74, 49-139). MAP kinases can be activated by growth factors such as EGF, and cytokines such as TNF-alpha. In response to EGF, Ras becomes activated and recruits Raf1 to the membrane where Raf1 is activated by mechanisms that may involve phosphorylation and conformational changes (Morrison, D. K., and Cutler, R. E. (1997) Curr. Opin. Cell Biol. 9, 174-179). Active Raf1 phosphorylates MEK1 which in turn phosphorylates and activates the ERKs.

Tyrosine Protein Kinase Group

The tyrosine kinase group encompass both cytoplasmic (e.g. src) as well as transmembrane receptor tyrosine kinases (e.g. EGF receptor). These kinases play a pivotal role in the signal transduction processes that mediate cell proliferation, differentiation and apoptosis. One of the sequences, 17000030181412, is related to the human RET kinase. Mutations of the RET gene, encoding a receptor tyrosine kinase, have been associated with the inherited cancer syndromes MEN 2A and MEN 2B. They have also further been associated with both familial and sporadic medullary thyroid carcinomas. The kinase activity can be aberrantly activated by missense mutations affecting cysteine residues within the extracellular domain, leading to potent oncogenicity (Oncogene 1999 Aug. 26;18(34):4833-8).

STE Group

The STE family refers to the 3 classes of protein kinases that lie sequentially upstream of the MAPKs. This group includes STE7 (MEK or MAPKK) kinases, STE11 (MEKK or MAPKKK) kinases and STE20 (MEKKK) kinases. In humans, several protein kinase families that bear only distant homology with the STE11 family also operate at the level of MAPKKKs including RAF, MLK, TAK1, and COT. Since crosstalk takes place between protein kinases functioning at different levels of the MAPK cascade, the large number of STE family kinases could translate into an enormous potential for upstream signal specificity.

The prototype STE20 from baker's yeast is regulated by a hormone receptor, signaling to directly affect cell cycle progression through modulation of CDK activity. It also coordinately regulates changes in the cytoskeleton and in transcriptional programs in a bifurcating pathway. In a similar way, the homologous kinases in humans are likely to play a role in extracellular regulation of growth, cell adhesion and migration, and changes in transcriptional programs, all three of which have critical roles in tumorigenesis. Mammalian STE20-related protein kinases have been implicated in response to growth factors or cytokines, oxidative-, UV-, or irradiation-related stress pathways, inflammatory signals (e.g. TNFα), apoptotic stimuli (e.g. Fas), T and B cell costimulation, the control of cytoskeletal architecture, and cellular transformation. Typically the STE20-related kinases serve as upstream regulators of MAPK cascades. Examples include: HPK1, a protein-serine/threonine kinase (STK) that possesses a STE20-like kinase domain that activates a protein kinase pathway leading to the stress-activated protein kinase SAPK/JNK; PAK1, an STK with an upstream CDC42-binding domain that interacts with Rac and plays a role in cellular transformation through the Ras-MAPK pathway; and murine NIK, which interacts with upstream receptor tyrosine kinases and connects with downstream STE11 -family kinases.

NEK kinases are related to NIMA, which is required for entry into mitosis in the filamentous fungus A. nidulans. Mutations in the nimA gene cause the nim (never in mitosis) G2 arrest phenotype in this fungus (Fry, A. M. and Nigg, E. A. (1995) Current Biology 5: 1122-1125). Several observations suggest that higher eukaryotes may have a NIMA functional counterpart(s): (1) expression of a dominant-negative form of NIMA in HeLa cells causes a G2 arrest; (2)overexpression of NIMA causes chromatin condensation, not only in A. nidulans, but also in yeast, Xenopus oocytes and HeLa cells (Lu, K. P. and Hunter, T. (1995) Prog. Cell Cycle Res. 1, 187-205); (3) NIMA when expressed in mammalian cells interacts with pin 1, a prolyl-prolyl isomerase that functions in cell cycle regulation (Lu, K. P. et al. (1996) Nature 380, 544-547); (4) okadaic acid inhibitor studies suggests the presence of cdc2-independent mechanism to induce mitosis (Ghosh, S. et al.(1998) Exp. Cell Res. 242, 1-9) and (5) a NIMA-like kinase (fin1) exists in another eukaryote besides Aspergillus, Saccharomyces pombe (Krien, M. J. E. et al.(1998) J. Cell Sci. 111, 967-976). Four mammalian NIMA-like kinases have been identified. NEK1, NEK2, NEK3 and NRK2. Despite the similarity of the NIMA-related kinases to NIMA over the catalytic region, the mammalian kinases are structurally different to NIMA over the extracatalytic regions. In addition the mammalian kinases are unable to complement the nim phenotype in Aspergillus nimA mutants. These observations lead to the following three possibilities: 1) the mammalian NIMA homologue remains unidentified; 2) there is no NIMA homologue in higher eukaryotes; 3) the biological function of NIMA is carried out by multiple, related kinases in higher eukaryotes. The elucidation and biological characterization of additional mammalian NIMA- and NEK-related kinases should assist in elucidating this question.

Casein Kinase 1 Group

The CK1 family represents a distant branch of the protein kinase family. The hallmarks of protein kinase subdomains VIII and IX are difficult to identify. One or more forms are ubiquitously distributed in mammalian tissues and cell lines. CK1 kinases are found in cytoplasm, in nuclei, membrane-bound, and associated with the cytoskeleton. Splice variants differ in their subcellular distribution.

“Other” Group

Several families cluster within a group of unrelated kinases termed “Other” . Included are: CHK1; Elongation 2 factor kinases (EIFK); homologues of the yeast sterile family kinases (STE), which refers to 3 classes of kinases which lie sequentially upstream of the MAPKs; Calcium-calmodulin kinase kinases (CAMKK); dual-specific tyrosine kinases (DYRK); IkB kinases (IKK); Integrin receptor kinase (IRAK); endoribonuclease-associated kinases (IRE); Mixed lineage kinase (MLK); LIM-domain containing kinase (LIMK); MOS; PIM; Receptor interacting kinase (RIP); SR-protein specific kinase (SRPK); RAF; Serine-threonine kinase receptors (STKR); TAK1; Testis specific kinase (TSK); tousled-related kinase (TSL); UNC51-related kinase (UNC); VRK; WEE; mitotic kinases (BUB1, AURORA, PLK, and NIMA/NEK); several families that are close homologues to worm (C26C2.1, YQ09, ZC581.9, YFL033c, C24A1.3); Drosophila (SLOB), or yeast (YDOD_sp, YGR262_sc) kinases; and others that are “unique,” that is, those which do not cluster into any obvious family. Additional families are even less well defined and first were identified in lower eukaryotes such as yeast or worms (YNL020, YPL236, YQ09, YWY3, SCY1, C01H6.9, C26C2.1)

RIP2 is a serine-threonine kinase associated with the tumor necrosis factor (TNF) receptor complex and is implicated in the activation of NF-kappa B and cell death in mammalian cells. It has recently been demonstrated that RIP2 activates the MAPK pathway (Navas, et al., J Biol. Chem. 1999 Nov. 19;274(47):33684-33690). RIP2 activates AP-1 and serum response element regulated expression by inducing the activation of the Elk1 transcription factor. RIP2 directly phosphorylates and activates ERK2 in vivo and in vitro. RIP2 in turn is activated through its interaction with Ras-activated Raf1. These results highlight the integrated nature of kinase signaling pathway.

The tousled (TSL) kinase was first identified in the plant Arabidopsis thaliana. TSL encodes a serine/threonine kinase that is essential for proper flower development. Human tousled-like kinases (Tlks) are cell-cycle-regulated enzymes, displaying maximal activities during S phase. This regulated activity suggests that Tlk function is linked to ongoing DNA replication (Sillje, et al., EMBO J 1999 Oct. 15; 18(20):5691-5702).

Atypical Protein Kinase Group

There are several proteins with protein kinase activity that appear structurally unrelated to the eukaryotic protein kinases. These include; Dictyostelium myosin heavy chain kinase A (MHCKA), Physarum polycephalum actin-fragmin kinase, the human A6 PTK, human BCR, mitochondrial pyruvate dehydrogenase and branched chain fatty acid dehydrogenase kinase, and the prokaryotic “histidine” protein kinase family. The slime mold, worm, and human eEF-2 kinase homologues have all been demonstrated to have protein kinase activity, yet they bear little resemblance to conventional protein kinases except for the presence of a putative GxGxxG ATP-binding motif.

The so-called histidine kinases are abundant in prokaryotes, with more than 20 representatives in E. coli, and have also been identified in yeast, molds, and plants. In response to external stimuli, these kinases act as part of two-component systems to regulate DNA replication, cell division, and differentiation through phosphorylation of an aspartate in the target protein. To date, no “histidine” kinases have been identified in metazoans, although mitochondrial pyruvate dehydrogenase (PDK) and branched chain alpha-ketoacid dehydrogenase kinase (BCKD kinase), are related in sequence. PDK and BCKD kinase represent a unique family of atypical protein kinases involved in regulation of glycolysis, the citric acid cycle, and protein synthesis during protein malnutrition. Structurally they conserve only the C-terminal portion of “histidine” kinases including the G box regions. BCKD kinase phosphorylates the E1a subunit of the BCKD complex on Ser-293, proving it to be a functional protein kinase. Although no bona fide “histidine” kinase has yet been identified in humans, they do contain PDK.

Several other proteins contain protein kinase-like homology including: receptor guanylyl cyclases, diacylglycerol kinases, choline/ethanolamine kinases, and YLK1-related antibiotic resistance kinases. Each of these families contain short motifs that were recognized by our profile searches with low scoring E-values, but a priori would not be expected to function as protein kinases. Instead, the similarity could simply reflect the modular nature of protein evolution and the primal role of ATP binding in diverse phosphotransfer enzymes. However, two recent papers on a bacterial homologue of the YLK1 family suggests that the aminoglycoside phosphotransferases (APHs) are structurally and functionally related to protein kinases. There are over 40 APHs identified from bacteria that are resistant to aminoglycosides such as kanamycin, gentamycin, or amikacin. The crystal structure of one well characterized APH reveals that it shares greater than 40% structural identity with the 2 lobed structure of the catalytic domain of cAMP-dependent protein kinase (PKA), including an N-terminal lobe composed of a 5-stranded antiparallel beta sheet and the core of the C-terminal lobe including several invariant segments found in all protein kinases. APHs lack the GxGxxG normally present in the loop between beta strands 1 and 2 but contain 7 of the 12 strictly conserved residues present in most protein kinases, including the HGDxxxN (SEQ ID NO. 115) signature sequence in kinase subdomain VIB. Furthermore, APH also has been shown to exhibit protein-serine/threonine kinase activity, suggesting that other YLK-related molecules may indeed be functional protein kinases.

The eukaryotic lipid kinases (PI3Ks, PI4Ks, and PIPKs) also contain several short motifs similar to protein kinases, but otherwise share minimal primary sequence similarity. However, once again structural analysis of PIPKII-beta defines a conserved ATP-binding core that is strikingly similar to conventional protein kinases. Three residues are conserved among all of these enzymes including (relative to the PKA sequence) Lys-72 which binds the gamma-phosphate of ATP, Asp-166 which is part of the HRDLK (SEQ ID NO. 116) motif and Asp-184 from the conserved Mg⁺⁺ or Mn⁺⁺ binding DFG motif. The worm genome contains 12 phosphatidylinositol kinases, including 3 PI3-kinases, 2 PI4-kinases, 3 PIP5-kinases, and 4 PI3-kinase-related kinases. The latter group has 4 mammalian members (DNA-PK, FRAP/TOR, ATM, and ATR), which have been shown to participate in the maintenance of genomic integrity in response to DNA damage, and exhibit true protein kinase activity, raising the possibility that other PI-kinases may also act as protein kinases. Regardless of whether they have true protein kinase activity, PI3-kinases are tightly linked to protein kinase signaling, as evidenced by their involvement downstream of many growth factor receptors and as upstream activators of the cell survival response mediated by the AKT protein kinase.

### SUMMARY OF THE INVENTION

The present invention relates, in part, to human protein kinases and protein kinase-like enzymes identified from genomic sequencing.

Tyrosine and serine/threonine kinases (PTK's and STK's) have been identified and their protein sequence predicted as part of the instant invention. Mammalian members of these families were identified through the use of a bioinformatics strategy. The partial or complete sequences of these kinases are presented here, together with their classification, predicted or deduced protein structure.

One aspect of the invention features an identified, isolated, enriched, or purified nucleic acid molecule encoding a kinase polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114.

The term “identified” in reference to a nucleic acid is meant that a sequence was selected from a genomic, EST, or cDNA sequence database based on it being predicted to encode a portion of a previously unknown or novel protein kinase.

By “isolated,” in reference to nucleic acid, is meant a polymer of 10 (preferably 21, more preferably 39, most preferably 75) or more nucleotides conjugated to each other, including DNA and RNA that is isolated from a natural source or that is synthesized as the sense or complementary antisense strand. In certain embodiments of the invention, longer nucleic acids are preferred, for example those of 300, 600, 900, 1200, 1500, or more nucleotides and/or those having at least 50%, 60%, 75%, 80%, 85%, 90%, 95% or 99% identity to a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57.

The isolated nucleic acid of the present invention is unique in the sense that it is not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2- to 5-fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level this level should be at least 2- to 5-fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

By a “kinase polypeptide” is meant 32 (preferably 40, more preferably 45, most preferably 55) or more contiguous amino acids in a polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. In certain aspects, polypeptides of 100, 200, 300, 400, 450, 500, 550, 600, 700, 800, 900 or more amino acids are preferred. The kinase polypeptide can be encoded by a full-length nucleic acid sequence or any portion (e.g., a “fragment” as defined herein) of the full-length nucleic acid sequence, so long as a functional activity of the polypeptide is retained, including, for example, a catalytic domain, as defined herein, or a portion thereof. One of skill in the art would be able to select those catalytic domains, or portions thereof, which exhibit a kinase or kinase-like activity, e.g., catalytic activity, as defined herein. It is well known in the art that due to the degeneracy of the genetic code numerous different nucleic acid sequences can code for the same amino acid sequence. Equally, it is also well known in the art that conservative changes in amino acid can be made to arrive at a protein or polypeptide which retains the functionality of the original. Such substitutions may include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making amino acid exchanges which have only slight, if any, effects on the overall protein can be found in Bowie et al., Science, 1990, 247, 1306-1310, which is incorporated herein by reference in its entirety including any figures, tables, or drawings. In all cases, all permutations are intended to be covered by this disclosure.

The amino acid sequence of a kinase peptide of the invention will be substantially similar to a sequence having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, or the corresponding full-length amino acid sequence, or fragments thereof.

A sequence that is substantially similar to a sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:l l0, SEQ ID NO:111, SEQ ID NO:112, SEQ ID NO:113, and SEQ ID NO:114, will preferably have at least 90% identity (more preferably at least 95% and most preferably 99-100%) to the sequence.

By “identity” is meant a property of sequences that measures their similarity or relationship. Identity is measured by dividing the number of identical residues by the total number of residues and gaps and multiplying the product by 100. “Gaps” are spaces in an alignment that are the result of additions or deletions of amino acids. Thus, two copies of exactly the same sequence have 100% identity, but sequences that are less highly conserved, and have deletions, additions, or replacements, may have a lower degree of identity. Those skilled in the art will recognize that several computer programs are available for determining sequence identity using standard parameters, for example Gapped BLAST or PSI-BLAST (Altschul, et al. (1997) Nucleic Acids Res. 25:3389-3402), BLAST (Altschul, et al. (1990) J. Mol. Biol. 215:403-410), and Smith-Waterman (Smith, et al. (1981) J. Mol. Biol. 147:195-197). Preferably, the default settings of these programs will be employed, but those skilled in the art recognize whether these settings need to be changed and know how to make the changes.

“Similarity” is measured by dividing the number of identical residues plus the number of conservatively substituted residues (see Bowie, et al. Science, 1999), 247, 1306-1310, which is incorporated herein by reference in its entirety, including any drawings, figures, or tables) by the total number of residues and gaps and multiplying the product by 100.

In preferred embodiments, the invention features isolated, enriched, or purified nucleic acid molecules encoding a kinase polypeptide comprising a nucleotide sequence that: (a) encodes a polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114; (b) is the complement of the nucleotide sequence of (a); (c) hybridizes under highly stringent conditions to the nucleotide molecule of (a) and encodes a naturally occurring kinase polypeptide; (d) encodes a polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104,SEQ ID NO:105,SEQ ID NO:106,SEQ ID NO:107,SEQ ID NO:108,SEQ ID NO:109, SEQ ID NO:110, SEQ ID NO:111, SEQ ID NO:112, SEQ ID NO:113, and SEQ ID NO:114, except that it lacks one or more, but not all, of the domains selected from the group consisting of an N-terminal domain, a catalytic domain, a C-terminal catalytic domain, a C-terminal domain, a coiled-coil structure region, a proline-rich region, a spacer region, and a C-terminal tail; and (e) is the complement of the nucleotide sequence of (d).

The term “complement” refers to two nucleotides that can form multiple favorable interactions with one another. For example, adenine is complementary to thymine as they can form two hydrogen bonds. Similarly, guanine and cytosine are complementary since they can form three hydrogen bonds. A nucleotide sequence is the complement of another nucleotide sequence if all of the nucleotides of the first sequence are complementary to all of the nucleotides of the second sequence.

Various low or high stringency hybridization conditions may be used depending upon the specificity and selectivity desired. These conditions are well known to those skilled in the art. Under stringent hybridization conditions only highly complementary nucleic acid sequences hybridize. Preferably, such conditions prevent hybridization of nucleic acids having more than 1 or 2 mismatches out of 20 contiguous nucleotides, more preferably, such conditions prevent hybridization of nucleic acids having more than 1 or 2 mismatches out of 50 contiguous nucleotides, most preferably, such conditions prevent hybridization of nucleic acids having more than 1 or 2 mismatches out of 100 contiguous nucleotides. In some instances, the conditions may prevent hybridization of nucleic acids having more than 5 mismatches in the full-length sequence.

By stringent hybridization assay conditions is meant hybridization assay conditions at least as stringent as the following: hybridization in 50% formamide, 5×SSC, 50 mM NaH2PO4, pH 6.8, 0.5% SDS, 0.1 mg/mL sonicated salmon sperm DNA, and 5× Denhardt's solution at 42° C. overnight; washing with 2×SSC, 0.1% SDS at 45° C.; and washing with 0.2×SSC, 0.1% SDS at 45° C. Under some of the most stringent hybridization assay conditions, the second wash can be done with 0.1×SSC at a temperature up to 70° C. (Berger et al. (1987) Guide to Molecular Cloning Techniques pg 421, hereby incorporated by reference herein in its entirety including any figures, tables, or drawings.). However, other applications may require the use of conditions falling between these sets of conditions. Methods of determining the conditions required to achieve desired hybridizations are well known to those with ordinary skill in the art, and are based on several factors, including but not limited to, the sequences to be hybridized and the samples to be tested. Washing conditions of lower stringency frequently utilize a lower temperature during the washing steps, such as 65° C., 60° C., 55° C., 50° C., or 42° C.

The term “domain” refers to a region of a polypeptide which serves a particular function. For instance, N-terminal or C-terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

The term “N-terminal domain” refers to the extracatalytic region located between the initiator methionine and the catalytic domain of the protein kinase. The N-terminal domain can be identified following a Smith-Waterman alignment of the protein sequence against the non-redundant protein database to define the N-terminal boundary of the catalytic domain. Depending on its length, the N-terminal domain may or may not play a regulatory role in kinase function. An example of a protein kinase whose N-terminal domain has been shown to play a regulatory role is PAK65, which contains a CRIB motif used for Cdc42 and rac binding (Burbelo, P. D. et al. (1995) J. Biol. Chem. 270, 29071-29074).

The term “catalytic domain” refers to a region of the protein kinase that is typically 25-300 amino acids long and is responsible for carrying out the phosphate transfer reaction from a high-energy phosphate donor molecule such as ATP or GTP to itself (autophosphorylation) or to other proteins (exogenous phosphorylation). The catalytic domain of protein kinases is made up of 12 subdomains that contain highly conserved amino acid residues, and are responsible for proper polypeptide folding and for catalysis. The catalytic domain can be identified following a Smith-Waterman alignment of the protein sequence against the non-redundant protein database.

The term “catalytic activity”, as used herein, defines the rate at which a kinase catalytic domain phosphorylates a substrate. Catalytic activity can be measured, for example, by determining the amount of a substrate converted to a phosphorylated product as a function of time. Catalytic activity can be measured by methods of the invention by holding time constant and determining the concentration of a phosphorylated substrate after a fixed period of time. Phosphorylation of a substrate occurs at the active site of a protein kinase. The active site is normally a cavity in which the substrate binds to the protein kinase and is phosphorylated.

The term “substrate” as used herein refers to a molecule phosphorylated by a kinase of the invention. Kinases phosphorylate substrates on serine/threonine or tyrosine amino acids. The molecule may be another protein or a polypeptide.

The term “C-terminal domain” refers to the region located between the catalytic domain or the last (located closest to the C-terminus) functional domain and the carboxy-terminal amino acid residue of the protein kinase. By “functional” domain is meant any region of the polypeptide that may play a regulatory or catalytic role as predicted from amino acid sequence homology to other proteins or by the presence of amino acid sequences that may give rise to specific structural conformations (e.g. N-terminal domain). The C-terminal domain can be identified by using a Smith-Waterman alignment of the protein sequence against the non-redundant protein database to define the C-terminal boundary of the catalytic domain or of any functional C-terminal extracatalytic domain. Depending on its length and amino acid composition, the C-terminal domain may or may not play a regulatory role in kinase function. An example of a protein kinase whose C-terminal domain may play a regulatory role is PAK3 which contains a heterotrimeric G_{b} subunit-binding site near its C-terminus (Leeuw, T. et al. (1998) Nature, 391, 191-195). For the some of the kinases of the instant invention, the C-terminal domain may also comprise the catalytic domain (above).

The term “C-terminal tail” as used herein, refers to a C-terminal domain of a protein kinase, that by homology extends or protrudes past the C-terminal amino acid of its closest homolog. C-terminal tails can be identified by using a Smith-Waterman sequence alignment of the protein sequence against the non-redundant protein database, or by means of a multiple sequence alignment of homologous sequences using the DNAStar program Megalign. Depending on its length, a C-terminal tail may or may not play a regulatory role in kinase function.

The term “coiled-coil structure region” as used herein, refers to a polypeptide sequence that has a high probability of adopting a coiled-coil structure as predicted by computer algorithms such as COILS (Lupas, A. (1996) Meth. Enzymology 266:513-525). Coiled-coils are formed by two or three amphipathic α-helices in parallel. Coiled-coils can bind to coiled-coil domains of other polypeptides resulting in homo- or heterodimers (Lupas, A. (1991) Science 252:1162-1164). Coiled-coil-dependent oligomerization has been shown to be necessary for protein function including catalytic activity of serine/threonine kinases (Roe, J. et al. (1997) J. Biol. Chem. 272:5838-5845).

The term “proline-rich region” as used herein, refers to a region of a protein kinase whose proline content over a given amino acid length is higher than the average content of this amino acid found in proteins(i.e., >10%). Proline-rich regions are easily discernable by visual inspection of amino acid, sequences and quantitated by standard computer sequence analysis programs such as the DNAStar program EditSeq. Proline-rich regions have been demonstrated to participate in regulatory protein-protein interactions. Among these interactions, those that are most relevant to this invention involve the “PxxP” proline rich motif found in certain protein kinases (i.e., human PAK1) and the SH3 domain of the adaptor molecule Nck (Galisteo, M. L. et al. (1996) J. Biol. Chem. 271:20997-21000). Other regulatory interactions involving “PxxP” proline-rich motifs include the WW domain (Sudol, M. (1996) Prog. Biochys. Mol. Bio. 65:113-132).

The term “spacer region” as used herein, refers to a region of the protein kinase located between predicted functional domains. The spacer region has no detectable homology to any amino acid sequence in the database, and can be identified by using a Smith-Waterman alignment of the protein sequence against the non-redundant protein database to define the C- and N-terminal boundaries of the flanking functional domains. Spacer regions may or may not play a fundamental role in protein kinase function. Precedence for the regulatory role of spacer regions in kinase function is provided by the role of the src kinase spacer in inter-domain interactions (Xu, W. et al. (1997) Nature 385:595-602).

The term “insert” as used herein refers to a portion of a protein kinase that is absent from a close homolog. Inserts may or may not by the product alternative splicing of exons. Inserts can be identified by using a Smith-Waterman sequence alignment of the protein sequence against the non-redundant protein database, or by means of a multiple sequence alignment of homologous sequences using the DNAStar program Megalign. Inserts may play a functional role by presenting a new interface for protein-protein interactions, or by interfering with such interactions.

The term “signal transduction pathway” refers to the molecules that propagate an extracellular signal through the cell membrane to become an intracellular signal. This signal can then stimulate a cellular response. The polypeptide molecules involved in signal transduction processes are typically receptor and non-receptor protein tyrosine kinases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+ binding proteins, cAMP binding proteins, guanyl cyclases, adenylyl cyclases, NO generating proteins, nucleotide exchange factors, and transcription factors.

In other preferred embodiments, the invention features isolated, enriched, or purified nucleic acid molecules encoding kinase polypeptides, further comprising a vector or promoter effective to initiate transcription in a host cell. The invention also features recombinant nucleic acid, preferably in a cell or an organism. The recombinant nucleic acid may contain a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57, or a functional derivative thereof and a vector or a promoter effective to initiate transcription in a host cell. The recombinant nucleic acid can alternatively contain a transcriptional initiation region functional in a cell, a sequence complementary to an RNA sequence encoding a kinase polypeptide and a transcriptional termination region functional in a cell. Specific vectors and host cell combinations are discussed herein.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a kinase can be inserted into a vector by cutting the vector with restriction enzymes and ligating the two pieces together.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

In preferred embodiments, the isolated nucleic acid comprises, consists essentially of, or consists of a nucleic acid sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57, which encodes an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, a functional derivative thereof, or at least 35, 40, 45, 50, 60, 75, 100, 200, or 300 contiguous amino acids selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. The nucleic acid may be isolated from a natural source by cDNA cloning or by subtractive hybridization. The natural source may be mammalian, preferably human, preferably blood, semen or tissue, and the nucleic acid may be synthesized by the triester method or by using an automated DNA synthesizer.

The term “mammal” refers preferably to such organisms as mice, rats, rabbits, guinea pigs, sheep, and goats, more preferably to cats, dogs, monkeys, and apes, and most preferably to humans.

In yet other preferred embodiments, the nucleic acid is a conserved or unique region, for example those useful for: the design of hybridization probes to facilitate identification and cloning of additional polypeptides, the design of PCR probes to facilitate cloning of additional polypeptides, obtaining antibodies to polypeptide regions, and designing antisense oligonucleotides.

By “conserved nucleic acid regions”, are meant regions present on two or more nucleic acids encoding a kinase polypeptide, to which a particular nucleic acid sequence can hybridize under lower stringency conditions. Examples of lower stringency conditions suitable for screening for nucleic acid encoding kinase polypeptides are provided in Wahl et al. Meth. Enzym. 152:399-407 (1987) and in Wahl et al. Meth. Enzym. 152:415-423 (1987), which are hereby incorporated by reference herein in its entirety, including any drawings, figures, or tables. Preferably, conserved regions differ by no more than 5 out of 20 nucleotides, even more preferably 2 out of 20 nucleotides or most preferably 1 out of 20 nucleotides.

By “unique nucleic acid region” is meant a sequence present in a nucleic acid coding for a kinase polypeptide that is not present in a sequence coding for any other naturally occurring polypeptide. Such regions preferably encode 32 (preferably 40, more preferably 45, most preferably 55) or more contiguous amino acids, for example, an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. In particular, a unique nucleic acid region is preferably of mammalian origin.

Another aspect of the invention features a nucleic acid probe for the detection of nucleic acid encoding a kinase polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. in a sample. The nucleic acid probe contains a nucleotide base sequence that will hybridize to the sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57, or a functional derivative thereof.

In preferred embodiments, the nucleic acid probe hybridizes to nucleic acid encoding at least 12, 32, 75, 90, 105, 120, 150, 200, 250, 300 or 350 contiguous amino acids, wherein the nucleic acid sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57, or a functional derivative thereof.

Methods for using the probes include detecting the presence or amount of kinase RNA in a sample by contacting the sample with a nucleic acid probe under conditions such that hybridization occurs and detecting the presence or amount of the probe bound to kinase RNA. The nucleic acid duplex formed between the probe and a nucleic acid sequence coding for a kinase polypeptide may be used in the identification of the sequence of the nucleic acid detected (Nelson et al., in Nonisotopic DNA Probe Techniques, Academic Press, San Diego, Kricka, ed., p.275, 1992, hereby incorporated by reference herein in its entirety, including any drawings, figures, or tables). Kits for performing such methods may be constructed to include a container means having disposed therein a nucleic acid probe.

Methods for using the probes also include using these probes to find, for example, the full-length clone of each of the predicted kinases by techniques known to one skilled in the art. These clones will be useful for screening for small molecule compounds that inhibit the catalytic activity of the encoded kinase with potential utility in treating cancers, immune-related diseases and disorders, cardiovascular disease, brain or neuronal-associated diseases, and metabolic disorders. More specifically disorders including cancers of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, multiple sclerosis, and amyotrophic lateral sclerosis; viral or non-viral infections caused by HIV-1, HIV-2 or other viral- or prion-agents or fungal- or bacterial- organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

In another aspect, the invention describes a recombinant cell or tissue comprising a nucleic acid molecule encoding a kinase polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. In such cells, the nucleic acid may be under the control of the genomic regulatory elements, or may be under the control of exogenous regulatory elements including an exogenous promoter. By “exogenous” it is meant a promoter that is not normally coupled in vivo transcriptionally to the coding sequence for the kinase polypeptides.

The polypeptide is preferably a fragment of the protein encoded by an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. By “fragment,” is meant an amino acid sequence present in a kinase polypeptide. Preferably, such a sequence comprises at least 32, 45, 50, 60, 100, 200, or 300 contiguous amino acids of a sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109,SEQ ID NO:110,SEQ ID NO:111, SEQ ID NO:112,SEQ ID NO:113, and SEQ ID NO:114.

In another aspect, the invention features an isolated, enriched, or purified kinase polypeptide having the amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114.

By “isolated” in reference to a polypeptide is meant a polymer of 6 (preferably 12, more preferably 18, most preferably 25, 32, 40, or 50) or more amino acids conjugated to each other, including polypeptides that are isolated from a natural source or that are synthesized. In certain aspects longer polypeptides are preferred, such as those comprising 100, 200, 300, 400, 450, 500, 550, 600, 700, 800, 900 or more contiguous amino acids, including an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114.

The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term “significantly” here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

In preferred embodiments, the kinase polypeptide is a fragment of the protein encoded by an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. Preferably, the kinase polypeptide contains at least 32, 45, 50, 60, 100, 200, or 300 contiguous amino acids of a sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, or a functional derivative thereof.

In preferred embodiments, the kinase polypeptide-comprises an amino acid sequence having (a) an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114; and (b) an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, except that it lacks one or more of the domains selected from the group consisting of a C-terminal catalytic domain, an N-terminal domain, a catalytic domain, a C-terminal domain, a coiled-coil structure region, a proline-rich region, a spacer region, and a C-terminal tail.

The polypeptide can be isolated from a natural source by methods well-known in the art. The natural source may be mammalian, preferably human, preferably blood, semen or tissue, and the polypeptide may be synthesized using an automated polypeptide synthesizer.

In some embodiments the invention includes a recombinant kinase polypeptide having (a) an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. By “recombinant kinase polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

The polypeptides to be expressed in host cells may also be fusion proteins which include regions from heterologous proteins. Such regions may be included to allow, e.g., secretion, improved stability, or facilitated purification of the polypeptide. For example, a sequence encoding an appropriate signal peptide can be incorporated into expression vectors. A DNA sequence for a signal peptide (secretory leader) may be fused in-frame to the polynucleotide sequence so that the polypeptide is translated as a fusion protein comprising the signal peptide. A signal peptide that is functional in the intended host cell promotes extracellular secretion of the polypeptide. Preferably, the signal sequence will be cleaved from the polypeptide upon secretion of the polypeptide from the cell. Thus, preferred fusion proteins can be produced in which the N-terminus of a kinase polypeptide is fused to a carrier peptide.

In one embodiment, the polypeptide comprises a fusion protein which includes a heterologous region used to facilitate purification of the polypeptide. Many of the available peptides used for such a function allow selective binding of the fusion protein to a binding partner. A preferred binding partner includes one or more of the IgG binding domains of protein A are easily purified to homogeneity by affinity chromatography on, for example, IgG-coupled Sepharose. Alternatively, many vectors have the advantage of carrying a stretch of histidine residues that can be expressed at the N-terminal or C-terminal end of the target protein, and thus the protein of interest can be recovered by metal chelation chromatography. A nucleotide sequence encoding a recognition site for a proteolytic enzyme such as enterokinase, factor X procollagenase or thrombine may immediately precede the sequence for a kinase polypeptide to permit cleavage of the fusion protein to obtain the mature kinase polypeptide. Additional examples of fusion-protein binding partners include, but are not limited to, the yeast I-factor, the honeybee melatin leader in sf9 insect cells, 6-His tag, thioredoxin tag, hemaglutinin tag, GST tag, and OmpA signal sequence tag. As will be understood by one of skill in the art, the binding partner which recognizes and binds to the peptide may be any ion, molecule or compound including metal ions (e.g., metal affinity columns), antibodies, or fragments thereof, and any protein or peptide which binds the peptide, such as the FLAG tag.

In another aspect, the invention features an antibody (e.g., a monoclonal or polyclonal antibody) having specific binding affinity to a kinase polypeptide or a kinase polypeptide domain or fragment where the polypeptide is selected from the group having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. By “specific binding affinity” is meant that the antibody binds to the target kinase polypeptide with greater affinity than it binds to other polypeptides under specified conditions. Antibodies or antibody fragments are polypeptides that contain regions that can bind other polypeptides. The term “specific binding affinity” describes an antibody that binds to a kinase polypeptide with greater affinity than it binds to other polypeptides under specified conditions. Antibodies can be used to identify an endogenous source of kinase polypeptides, to monitor cell cycle regulation, and for immuno-localization of kinase polypeptides within the cell.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et al., Nature 256:495-497, 1975, and U.S. Pat. No. 4,376,110, both of which are hereby incorporated by reference herein in their entirety including any figures, tables, or drawings).

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

Antibodies or antibody fragments having specific binding affinity to a kinase polypeptide of the invention may be used in methods for detecting the presence and/or amount of kinase polypeptide in a sample by probing the sample with the antibody under conditions suitable for kinase-antibody immunocomplex formation and detecting the presence and/or amount of the antibody conjugated to the kinase polypeptide. Diagnostic kits for performing such methods may be constructed to include antibodies or antibody fragments specific for the kinase as well as a conjugate of a binding partner of the antibodies or the antibodies themselves.

An antibody or antibody fragment with specific binding affinity to a kinase polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a kinase polypeptide of the invention may be used in methods for detecting the presence and/or amount of kinase polypeptide in a sample by contacting the sample with the antibody under conditions such that an immunocomplex forms and detecting the presence and/or amount of the antibody conjugated to the kinase polypeptide. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

In another aspect, the invention features a hybridoma which produces an antibody having specific binding affinity to a kinase polypeptide or a kinase polypeptide domain, where the polypeptide is selected from the group having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. By “hybridoma” is meant an immortalized cell line that is capable of secreting an antibody, for example an antibody to a kinase of the invention. In preferred embodiments, the antibody to the kinase comprises a sequence of amino acids that is able to specifically bind a kinase polypeptide of the invention.

In another aspect, the present invention is also directed to kits comprising antibodies that bind to a polypeptide encoded by any of the nucleic acid molecules described above, and a negative control antibody.

The term “negative control antibody” refers to an antibody derived from similar source as the antibody having specific binding affinity, but where it displays no binding affinity to a polypeptide of the invention.

In another aspect, the invention features a kinase polypeptide binding agent able to bind to a kinase polypeptide selected from the group having (a) an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:1 10, SEQ ID NO:111, SEQ ID NO:112, SEQ ID NO:113, and SEQ ID NO:114. The binding agent is preferably a purified antibody that recognizes an epitope present on a kinase polypeptide of the invention. Other binding agents include molecules that bind to kinase polypeptides and analogous molecules that bind to a kinase polypeptide. Such binding agents may be identified by using assays that measure kinase binding partner activity, such as those that measure PDGFR activity.

The invention also features a method for screening for human cells containing a kinase polypeptide of the invention or an equivalent sequence. The method involves identifying the novel polypeptide in human cells using techniques that are routine and standard in the art, such as those described herein for identifying the kinases of the invention (e.g., cloning, Southern or Northern blot analysis, in situ hybridization, PCR amplification, etc.).

In another aspect, the invention features methods for identifying a substance that modulates kinase activity comprising the steps of: (a) contacting a kinase polypeptide selected from the group having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114 with a test substance; (b) measuring the activity of said polypeptide; and (c) determining whether said substance modulates the activity of said polypeptide. The skilled artisan will appreciate that the kinase polypeptides of the invention, including, for example, a portion of a full-length sequence such as a catalytic domain or a portion thereof, are useful for the identification of a substance which modulates kinase activity. Those kinase polypeptides having a functional activity (e.g., catalytic activity as defined herein) are useful for identifying a substance that modulates kinase activity.

The term “modulates” refers to the ability of a compound to alter the function of a kinase of the invention. A modulator preferably activates or inhibits the activity of a kinase of the invention depending on the concentration of the compound exposed to the kinase.

The term “modulates” also refers to altering the function of kinases of the invention by increasing or decreasing the probability that a complex forms between the kinase and a natural binding partner. A modulator preferably increases the probability that such a complex forms between the kinase and the natural binding partner, more preferably increases or decreases the probability that a complex forms between the kinase and the natural binding partner depending on the concentration of the compound exposed to the kinase, and most preferably decreases the probability that a complex forms between the kinase and the natural binding partner.

The term “activates” refers to increasing the cellular activity of the kinase. The term inhibit refers to decreasing the cellular activity of the kinase. Kinase activity is preferably the interaction with a natural binding partner.

The term “complex” refers to an assembly of at least two molecules bound to one another. Signal transduction complexes often contain at least two protein molecules bound to one another. For instance, a protein tyrosine receptor protein kinase, GRB2, SOS, RAF, and RAS assemble to form a signal transduction complex in response to a mitogenic ligand.

The term “natural binding partner” refers to polypeptides, lipids, small molecules, or nucleic acids that bind to kinases in cells. A change in the interaction between a kinase and a natural binding partner can manifest itself as an increased or decreased probability that the interaction forms, or an increased or decreased concentration of kinase/natural binding partner complex.

The term “contacting” as used herein refers to mixing a solution comprising the test compound with a liquid medium bathing the cells of the methods. The solution comprising the compound may also comprise another component, such as dimethyl sulfoxide (D[MSO), which facilitates the uptake of the test compound or compounds into the cells of the methods. The solution comprising the test compound may be added to the medium bathing the cells by utilizing a delivery apparatus, such as a pipette-based device or syringe-based device.

In another aspect, the invention features methods for identifying a substance that modulates kinase activity in a cell comprising the steps of: (a) expressing a kinase polypeptide in a cell, wherein said polypeptide is selected from the group having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114; (b) adding a test substance to said cell; and (c) monitoring a change in cell phenotype or the interaction between said polypeptide and a natural binding partner. The skilled artisan will appreciate that the kinase polypeptides of the invention, including, for example, a portion of a full-length sequence such as a catalytic domain or a portion thereof, are useful for the identification of a substance which modulates kinase activity. Those kinase polypeptides having a functional activity (e.g., catalytic activity as defined herein) are useful for identifying a substance that modulates kinase activity.

The term “expressing” as used herein refers to the production of kinases of the invention from a nucleic acid vector containing kinase genes within a cell. The nucleic acid vector is transfected into cells using well known techniques in the art as described herein.

Another aspect of the instant invention is directed to methods of identifying compounds that bind to kinase polypeptides of the present invention, comprising contacting the kinase polypeptides with a compound, and determining whether the compound binds the kinase polypeptides. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include, but are not limited to, compounds of extracellular, intracellular, biological or chemical origin.

The methods of the invention also embrace compounds that are attached to a label, such as a radiolabel (e.g., ¹²⁵I, ³⁵S, ³²P, ³³P, ³H), a fluorescence label, a chemiluminescent label, an enzymic label and an immunogenic label. The kinase polypeptides employed in such a test may either be free in solution, attached to a solid support, borne on a cell surface, located intracellularly or associated with a portion of a cell. One skilled in the art can, for example, measure the formation of complexes between a kinase polypeptide and the compound being tested. Alternatively, one skilled in the art can examine the diminution in complex formation between a kinase polypeptide and its substrate caused by the compound being tested.

Other assays can be used to examine enzymatic activity including, but not limited to, photometric, radiometric, HPLC, electrochemical, and the like, which are described in, for example, Enzyme Assays: A Practical Approach, eds. R. Eisenthal and M. J. Danson, 1992, Oxford University Press, which is incorporated herein by reference in its entirety.

Another aspect of the present invention is directed to methods of identifying compounds which modulate (i.e., increase or decrease) activity of a kinase polypeptide comprising contacting the kinase polypeptide with a compound, and determining whether the compound modifies activity of the kinase polypeptide. As described herein, the kinase polypeptides of the invention include a portion of a full-length sequence, such as a catalytic domain, as defined herein. In some instances, the kinase polypeptides of the invention comprise less than the entire catalytic domain, yet exhibit kinase or kinase-like activity. These compounds are also referred to as “modulators of protein kinases.” The activity in the presence of the test compound is measured to the activity in the absence of the test compound. Where the activity of a sample containing the test compound is higher than the activity in a sample lacking the test compound, the compound will have increased the activity. Similarly, where the activity of a sample containing the test compound is lower than the activity in the sample lacking the test compound, the compound will have inhibited the activity.

The present invention is particularly useful for screening compounds by using a kinase polypeptide in any of a variety of drug screening techniques. The compounds to be screened include, but are not limited to, extracellular, intracellular, biological or chemical origin. The kinase polypeptide employed in such a test may be in any form, preferably, free in solution, attached to a solid support, borne on a cell surface or located intracellularly. One skilled in the art can, for example, measure the formation of complexes between a kinase polypeptide and the compound being tested. Alternatively, one skilled in the art can examine the diminution in complex formation between a kinase polypeptide and its substrate caused by the compound being tested.

The activity of kinase polypeptides of the invention can be determined by, for example, examining the ability to bind or be activated by chemically synthesised peptide ligands. Alternatively, the activity of the kinase polypeptides can be assayed by examining their ability to bind metal ions such as calcium, hormones, chemokines, neuropeptides, neurotransmitters, nucleotides, lipids, odorants, and photons. Thus, modulators of the kinase polypeptide's activity may alter a kinase function, such as a binding property of a kinase or an activity such as signal transduction or membrane localization.

In various embodiments of the method, the assay may take the form of a yeast growth assay, an Aequorin assay, a Luciferase assay, a mitogenesis assay, a MAP Kinase activity assay, as well as other binding or function-based assays of kinase activity that are generally known in the art. In several of these embodiments, the invention includes any of the receptor and non-receptor protein tyrosine kinases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+binding proteins, cAMP binding proteins, guanyl cyclases, adenylyl cyclases, NO generating proteins, nucleotide exchange factors, and transcription factors. Biological activities of kinases according to the invention include, but are not limited to, the binding of a natural or a synthetic ligand, as well as any one of the functional activities of kinases known in the art. Non-limiting examples of kinase activities include transmembrane signaling of various forms, which may involve kinase binding interactions and/or the exertion of an influence over signal transduction.

The modulators of the invention exhibit a variety of chemical structures, which can be generally grouped into mimetics of natural kinase ligands, and peptide and non-peptide allosteric effectors of kinases. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries.

The use of cDNAs encoding kinases in drug discovery programs is well-known; assays capable of testing thousands of unknown compounds per day in high-throughput screens (HTSs) are thoroughly documented. The literature is replete with examples of the use of radiolabelled ligands in HTS binding assays for drug discovery (see Williams, Medicinal Research Reviews, 1991, 11, 147-184.; Sweetnam, et al., J. Natural Products, 1993, 56, 441-455 for review). Recombinant receptors are preferred for binding assay HTS because they allow for better specificity (higher relative purity), provide the ability to generate large amounts of receptor material, and can be used in a broad variety of formats (see Hodgson, Bio/Technology, 1992, 10, 973-980; each of which is incorporated herein by reference in its entirety).

A variety of heterologous systems is available for functional expression of recombinant receptors that are well known to those skilled in the art. Such systems include bacteria (Strosberg, et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Current Opinion in Biotechnology, 1997, 8, 629-634) and several mammalian cell lines (CHO, HEK293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

An expressed kinase can be used for HTS binding assays in conjunction with its defined ligand, in this case the corresponding peptide that activates it. The identified peptide is labeled with a suitable radioisotope, including, but not limited to, ¹²⁵I, ³H, ³⁵S or ³²P, by methods that are well known to those skilled in the art. Alternatively, the peptides may be labeled by well-known methods with a suitable fluorescent derivative (Baindur, et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11, 147-184.; Sweetnam, et al., J. Natural Products, 1993, 56, 441-455). Alternative methods include a scintillation proximity assay (SPA) or a FlashPlate format in which such separation is unnecessary (Nakayama, Cur. Opinion Drug Disc. Dev., 1998, 1, 85-91 Bossé, et al., J. Biomolecular Screening, 1998, 3, 285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Cur. Opinion Drug Disc. Dev., 1998, 1, 92-97).

The kinases and natural binding partners required for functional expression of heterologous kinase polypeptides can be native constituents of the host cell or can be introduced through well-known recombinant technology. The kinase polypeptides can be intact or chimeric. The kinase activation results in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca²⁺ concentration as measured by fluorescent dyes (Murphy, et al., Cur. Opinion Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80). Assays are also available for the measurement of common second but these are not generally preferred for HTS.

The invention contemplates a multitude of assays to screen and identify inhibitors of ligand binding to kinase polypeptides. In one example, the kinase polypeptide is immobilized and interaction with a binding partner is assessed in the presence and absence of a candidate modulator such as an inhibitor compound. In another example, interaction between the kinase polypeptide and its binding partner is assessed in a solution assay, both in the presence and absence of a candidate inhibitor compound. In either assay, an inhibitor is identified as a compound that decreases binding between the kinase polypeptide and its natural binding partner. Another contemplated assay involves a variation of the di-hybrid assay wherein an inhibitor of protein/protein interactions is identified by detection of a positive signal in a transformed or transfected host cell, as described in PCT publication number WO 95/20652, published Aug. 3, 1995 and is included by reference herein including any figures, tables, or drawings.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, while others are derived from natural products, and still others arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses both natural binding partners as described above as well as chimeric polypeptides, peptide modulators other than natural ligands, antibodies, antibody fragments, and modified compounds comprising antibody domains that are immunospecific for the expression product of the identified kinase gene.

Other assays may be used to identify specific peptide ligands of a kinase polypeptide, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a kinase gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

When the function of the kinase polypeptide gene product is unknown and no ligands are known to bind the gene product, the yeast two-hybrid assay can also be used to identify proteins that bind to the gene product. In an assay to identify proteins that bind to a kinase polypeptide, or fragment thereof, a fusion polynucleotide encoding both a kinase polypeptide (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No.5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

In preferred embodiments of the invention, methods of screening for compounds which modulate kinase activity comprise contacting test compounds with kinase polypeptides and assaying for the presence of a complex between the compound and the kinase polypeptide. In such assays, the ligand is typically labelled. After suitable incubation, free ligand is separated from that present in bound form, and the amount of free or uncomplexed label is a measure of the ability of the particular compound to bind to the kinase polypeptide.

In another embodiment of the invention, high throughput screening for compounds having suitable binding affinity to kinase polypeptides is employed. Briefly, large numbers of different small peptide test compounds are synthesised on a solid substrate. The peptide test compounds are contacted with the kinase polypeptide and washed. Bound kinase polypeptide is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with a kinase polypeptide. Radiolabeled competitive binding studies are described in A. H. Lin et al. Antimicrobial Agents and Chemotherapy, 1997, vol. 41, no. 10. pp. 2127-2131, the disclosure of which is incorporated herein by reference in its entirety.

In another aspect, the invention provides methods for treating a disease by administering to a patient in need of such treatment a substance that modulates the activity of a kinase polypeptide selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, as well as the full-length polypeptide thereof, or a portion of any of these sequences that retains functional activity, as described herein. Preferably the disease is selected from the group consisting of cancers, immune-elated diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial- organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

In preferred embodiments, the invention provides methods for treating or preventing a disease or disorder by administering to a patient in need of such treatment a substance that modulates the activity of a kinase polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, as well as the full-length polypeptide thereof, or a portion of any of these sequences that retains functional activity, as described herein. Preferably, the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV- 1, HIV-2 or other viral- or prion-agents or fungal- or bacterial- organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The invention also features methods of treating or preventing a disease or disorder by administering to a patient in need of such treatment a substance that modulates the activity of a kinase polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, as well as the full-length polypeptide thereof, or a portion of any of these sequences that retains functional activity, as described herein. Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial- organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The invention also features methods of treating or preventing a disease or disorder by administering to a patient in need of such treatment a substance that modulates the activity of a kinase polypeptide having an amino acid sequence selected from the group consisting those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, as well as the full-length polypeptide thereof, or a portion of any of these sequences that retains functional activity, as described herein. Preferably the disease is selected from the group consisting of immune-related diseases and disorders, cardiovascular disease, and cancer. More preferably these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial- organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection. Most preferably, the immune-related diseases and disorders are selected from the group consisting of rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplantation.

Substances useful for treatment of kinase-related disorders or diseases preferably show positive results in one or more in vitro assays for an activity corresponding to treatment of the disease or disorder in question (Examples of such assays are provided in the references in section VI, below; and in Example 7, herein). Examples of substances that can be screened for favorable activity are provided and referenced in section VI, below. The substances that modulate the activity of the kinases preferably include, but are not limited to, antisense oligonucleotides and inhibitors of protein kinases, as determined by methods and screens referenced in section VI and Example 7, below.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth, and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, or cell survival.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to neurodegenerative disorders, slow wound healing rates, and slow tissue grafting healing rates.

Abnormal cell survival conditions relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “aberration”, in conjunction with the function of a kinase in a signal transduction process, refers to a kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig, or goat, more preferably a monkey or ape, and most preferably a human.

In another aspect, the invention features methods for detection of a kinase polypeptide in a sample as a diagnostic tool for diseases or disorders, wherein the method comprises the steps of: (a) contacting the sample with a nucleic acid probe which hybridizes under hybridization assay conditions to a nucleic acid target region of a kinase polypeptide having an amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, arteriosclerosis, autoimmune disorders, organ transplantation, myocardial infarction, cardiomyopathies, stroke, renal failure, oxidative stress-related neurodegenerative disorders, and cancer.

The kinase “target region” is the nucleotide base sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57, or the corresponding full-length sequences, a functional derivative thereof, or a fragment thereof, to which the nucleic acid probe will specifically hybridize. Specific hybridization indicates that in the presence of other nucleic acids the probe only hybridizes detectably with the kinase of the invention's target region. Putative target regions can be identified by methods well known in the art consisting of alignment and comparison of the most closely related sequences in the database.

In preferred embodiments the nucleic acid probe hybridizes to a kinase target region encoding at least 6, 12, 75, 90, 105, 120, 150, 200, 250, 300 or 350 contiguous amino acids of a sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99,SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, or the corresponding full-length amino acid sequence, a portion of any of these sequences that retains functional activity, as described herein, or a functional derivative thereof. Hybridization conditions should be such that hybridization occurs only with the kinase genes in the presence of other nucleic acid molecules. Under stringent hybridization conditions only highly complementary nucleic acid sequences hybridize. Preferably, such conditions prevent hybridization of nucleic acids having more than 1 or 2 mismatches out of 20 contiguous nucleotides. Such conditions are defined supra.

The diseases for which detection of kinase genes in a sample could be diagnostic include diseases in which kinase nucleic acid (DNA and/or RNA) is amplified in comparison to normal cells. By “amplification” is meant increased numbers of kinase DNA or RNA in a cell compared with normal cells. In normal cells, kinases are typically found as single copy genes. In selected diseases, the chromosomal location of the kinase genes may be amplified, resulting in multiple copies of the gene, or amplification. Gene amplification can lead to amplification of kinase RNA, or kinase RNA can be amplified in the absence of kinase DNA amplification.

“Amplification” as it refers to RNA can be the detectable presence of kinase RNA in cells, since in some normal cells there is no basal expression of kinase RNA. In other normal cells, a basal level of expression of kinase exists, therefore in these cases amplification is the detection of at least 1-2-fold, and preferably more, kinase RNA, compared to the basal level.

The diseases that could be diagnosed by detection of kinase nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

The invention also features a method for detection of a kinase polypeptide in a sample as a diagnostic tool for a disease or disorder, wherein the method comprises: (a) comparing a nucleic acid target region encoding the kinase polypeptide in a sample, where the kinase polypeptide has an amino acid sequence selected from the group consisting those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, or one or more fragments thereof, with a control nucleic acid target region encoding the kinase polypeptide, or one or more fragments thereof; and (b) detecting differences in sequence or amount between the target region and the control target region, as an indication of the disease or disorder. Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial- organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The term “comparing” as used herein refers to identifying discrepancies between the nucleic acid target region isolated from a sample, and the control nucleic acid target region. The discrepancies can be in the nucleotide sequences, e.g. insertions, deletions, or point mutations, or in the amount of a given nucleotide sequence. Methods to determine these discrepancies in sequences are well-known to one of ordinary skill in the art. The “control” nucleic acid target region refers to the sequence or amount of the sequence found in normal cells, e.g. cells that are not diseased as discussed previously.

The summary of the invention described above is not limiting and other features and advantages of the invention will be apparent from the following detailed description of the invention, and from the claims.

### BRIEF DESCRIPTION OF THE FIGURES

FIGS. 1A-1EE shows the nucleotide sequences for human protein kinases oriented in a 5′ to 3′ direction (SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57).

FIGS. 2A-2L show the amino acid sequences for the human protein kinases encoded by SEQ ID No. 1-57 in the direction of translation (SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114). Some of the sequences encode predicted stop codons within the coding region, indicated by an ‘x.’

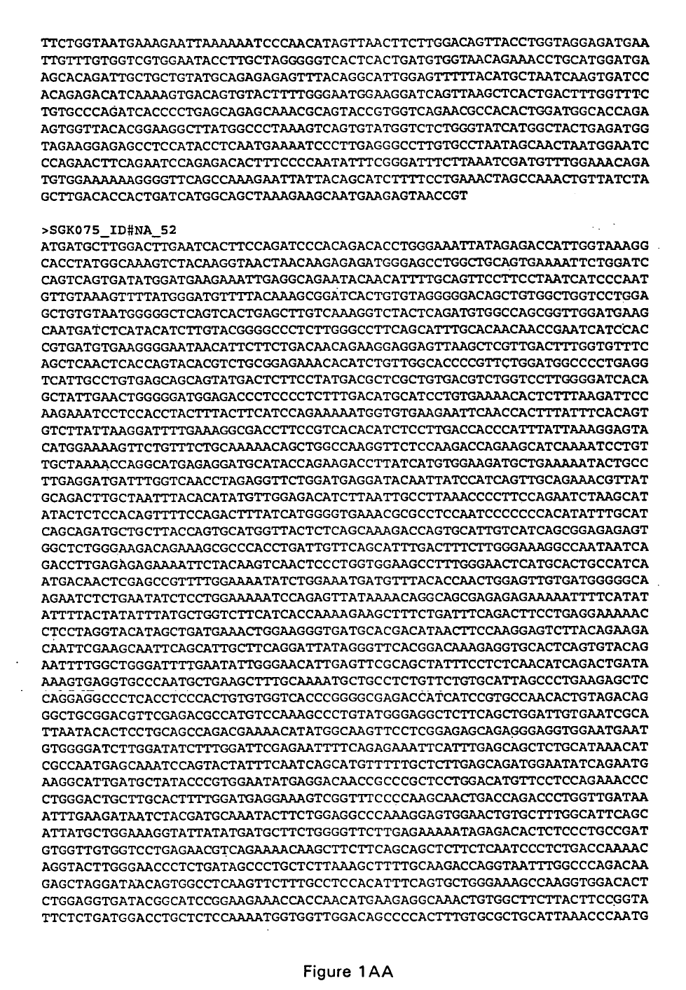

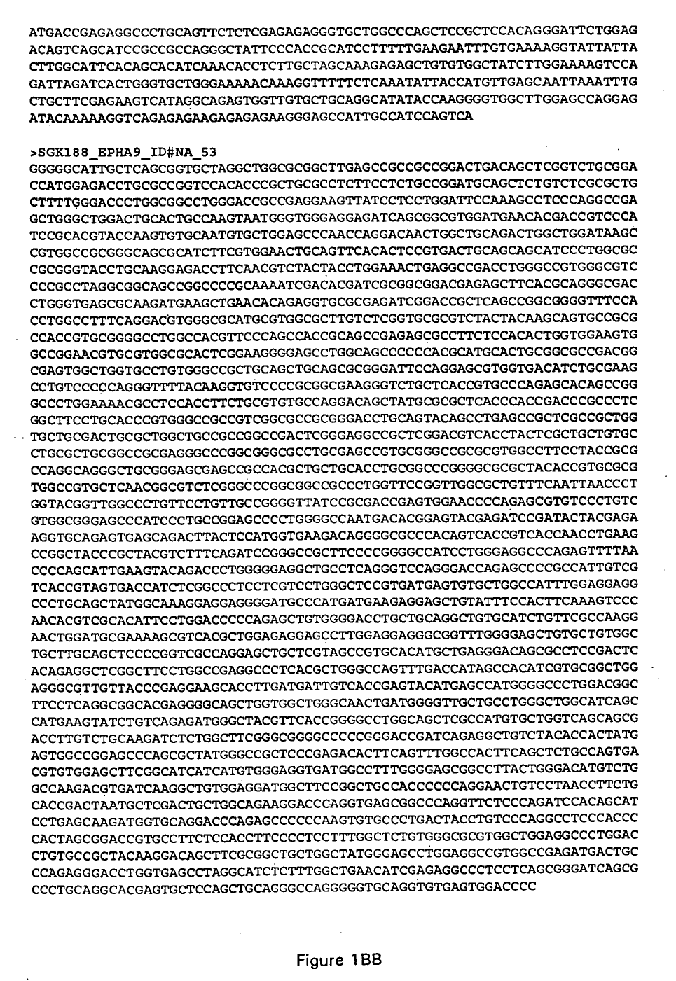

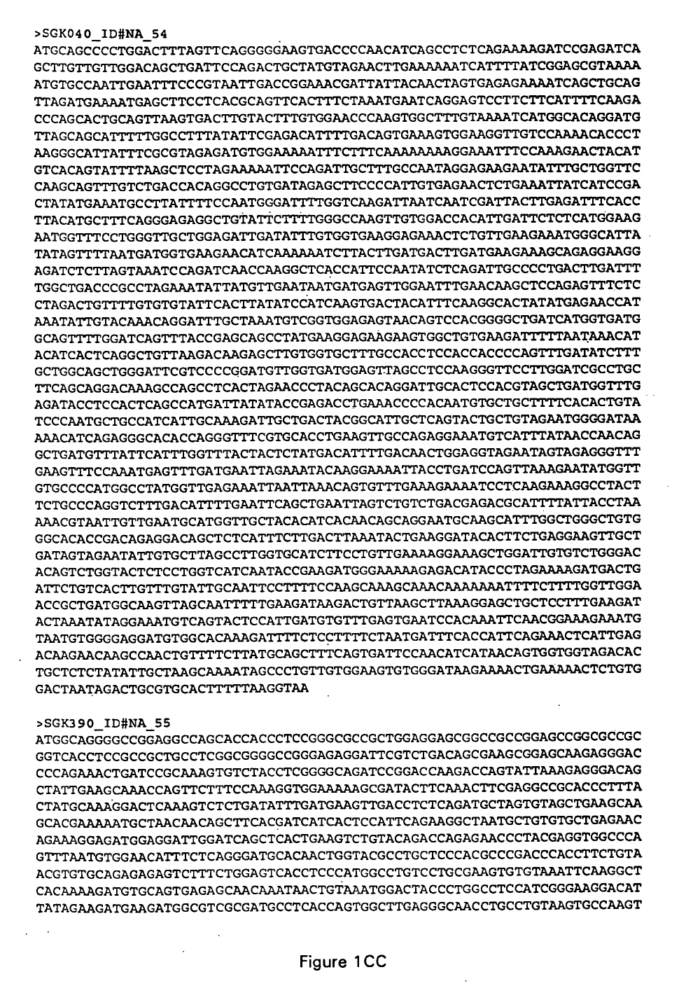

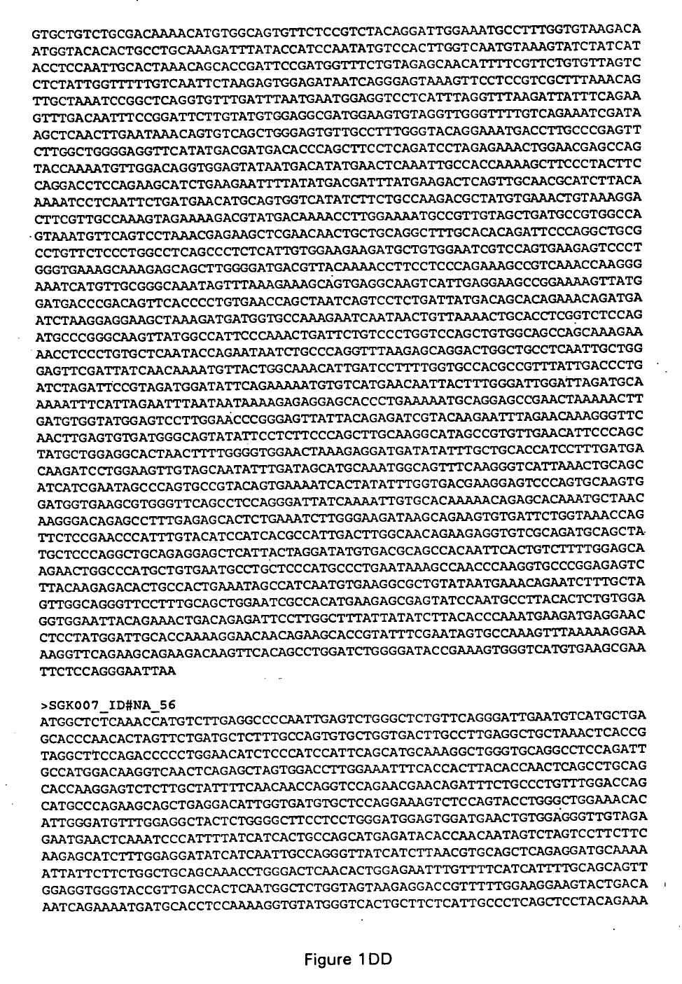

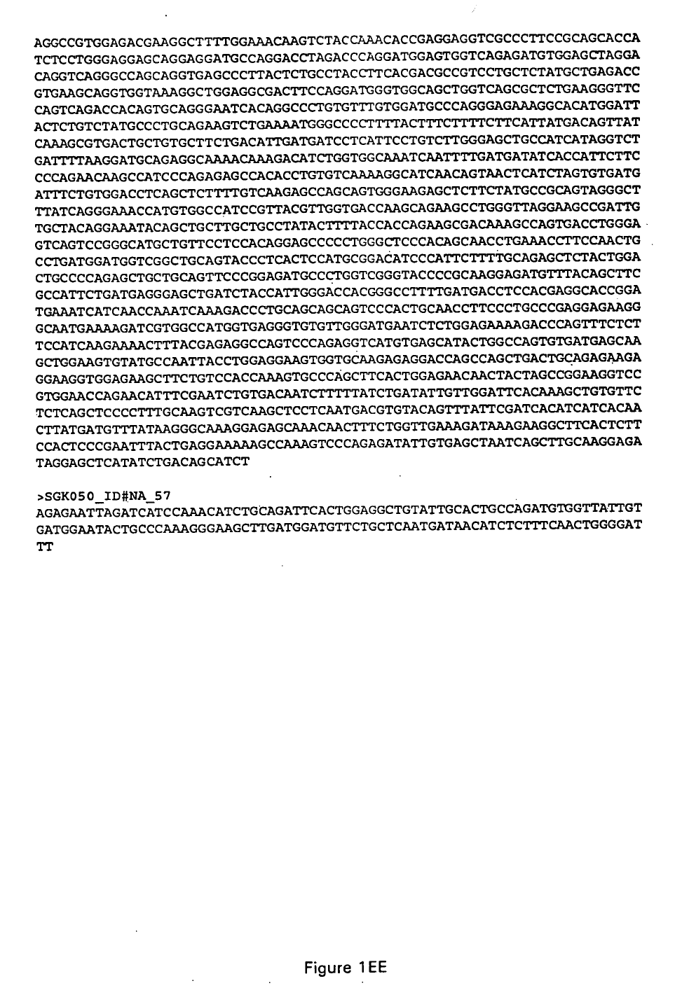

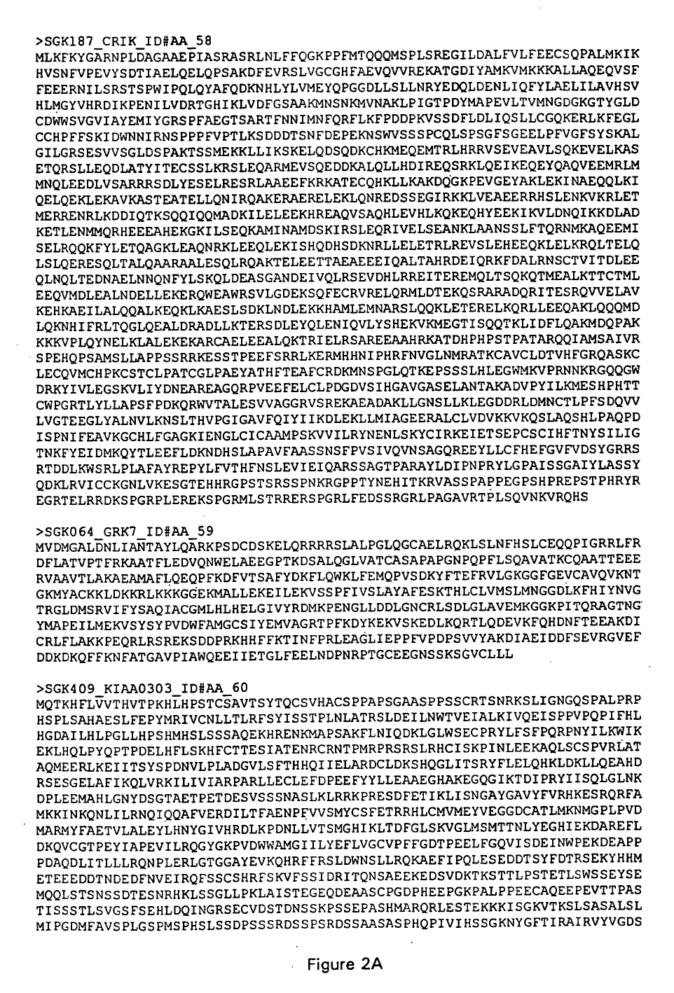

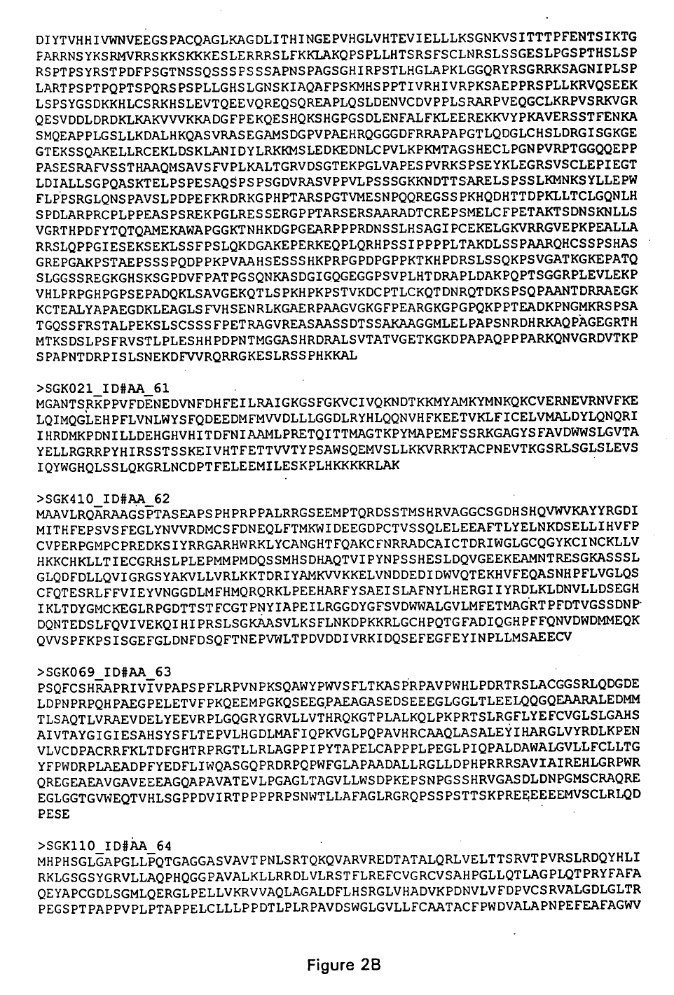

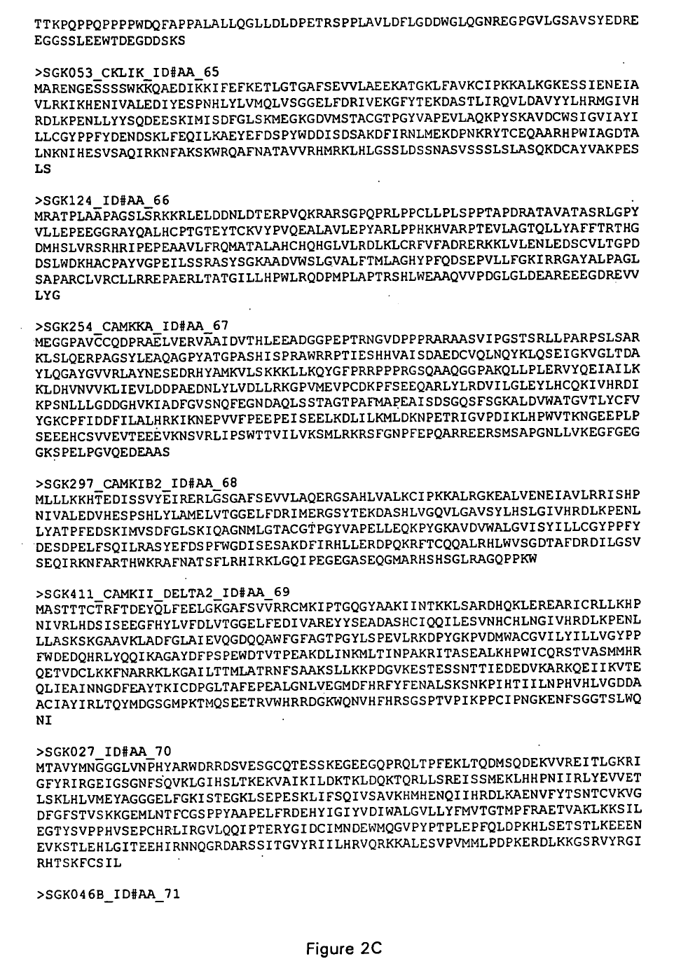

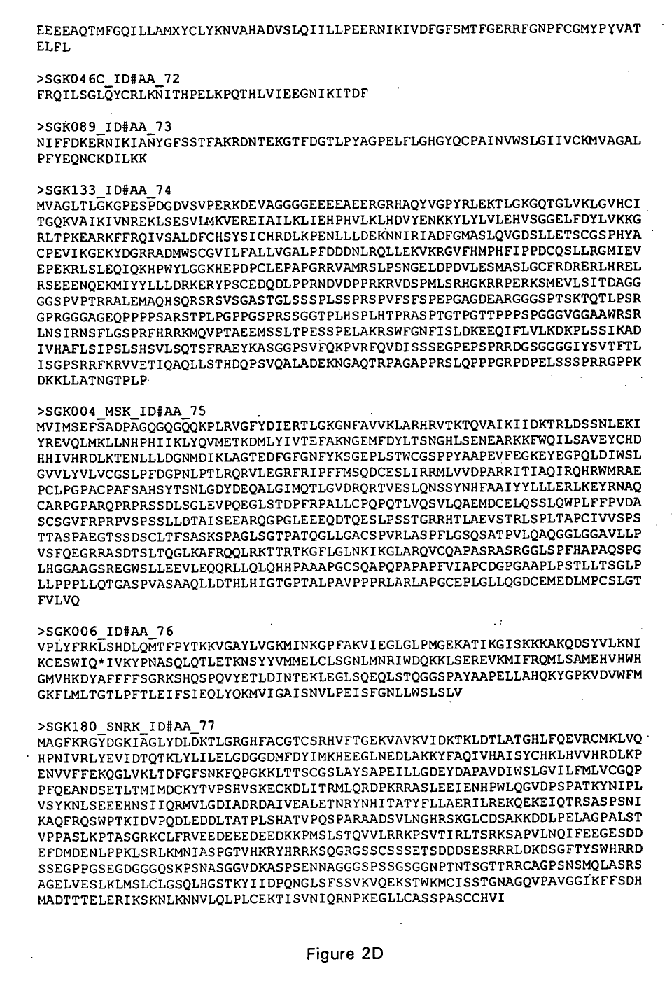

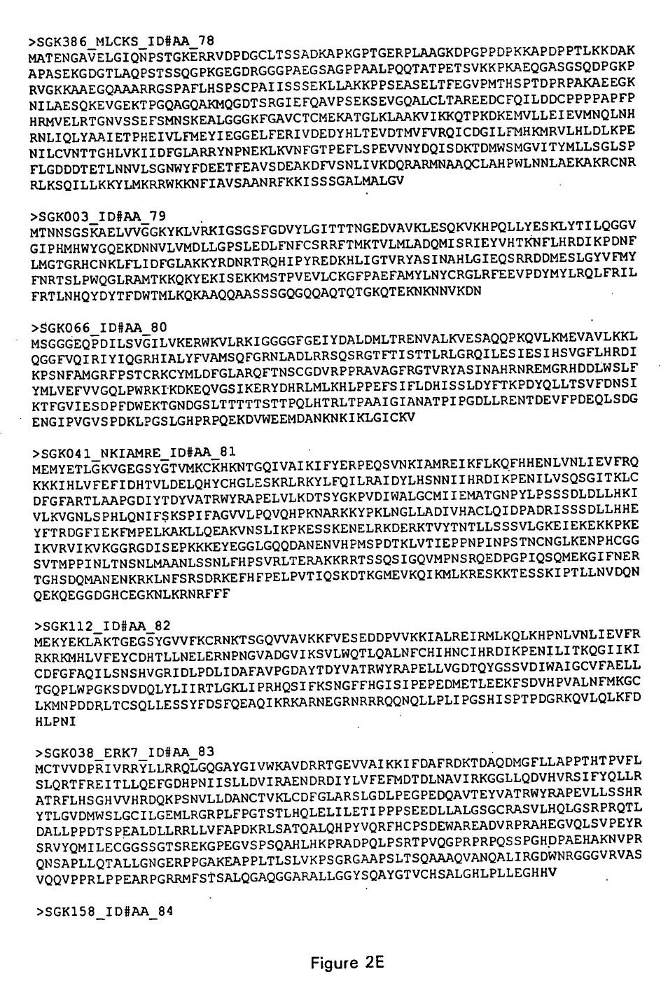

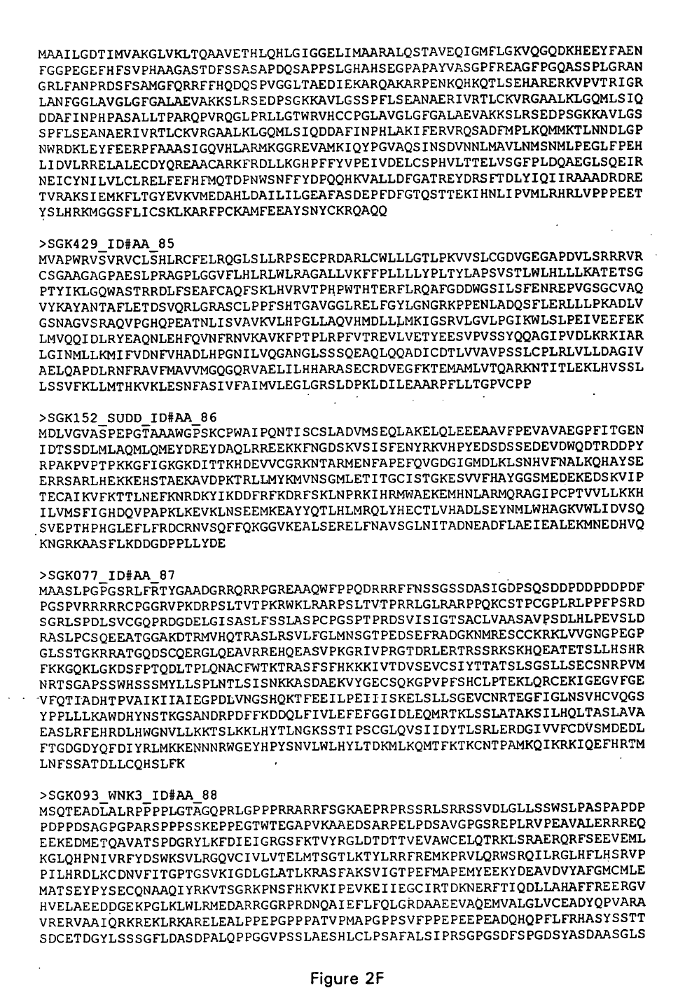

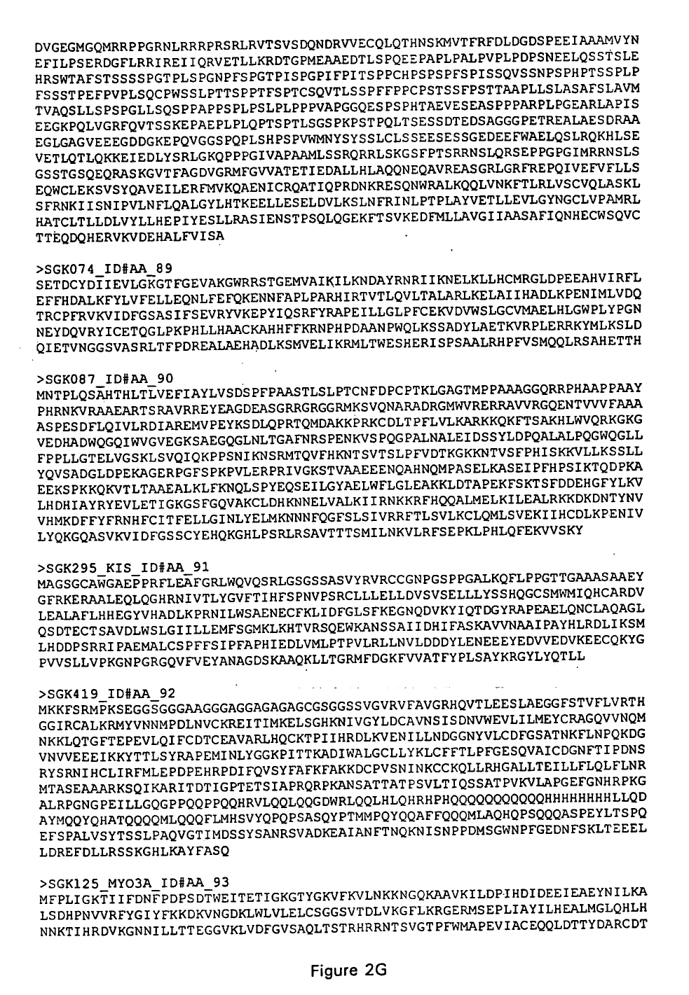

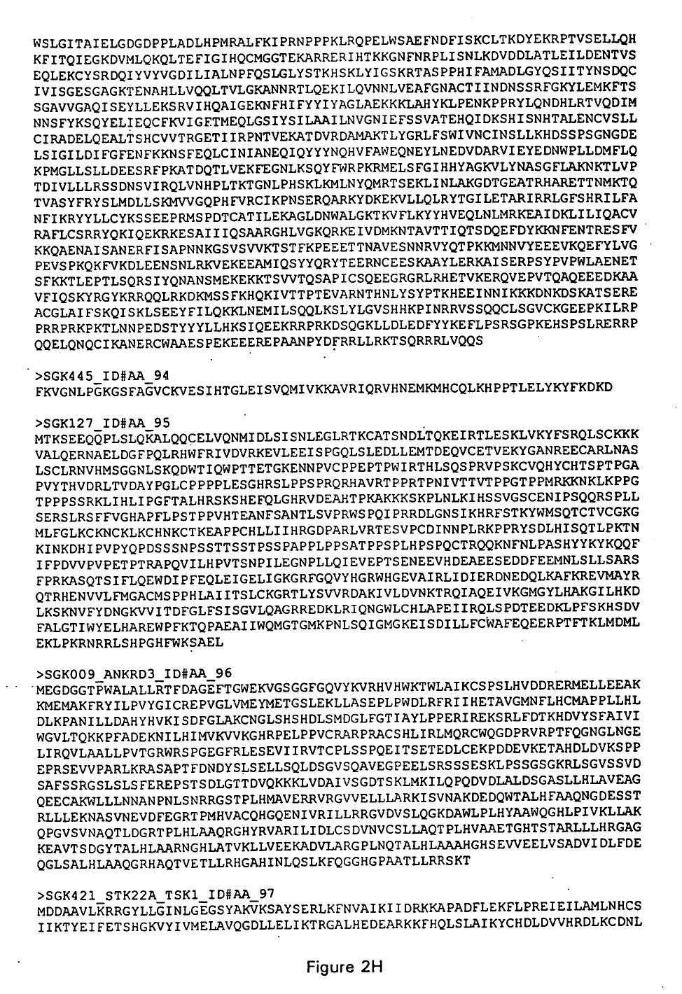

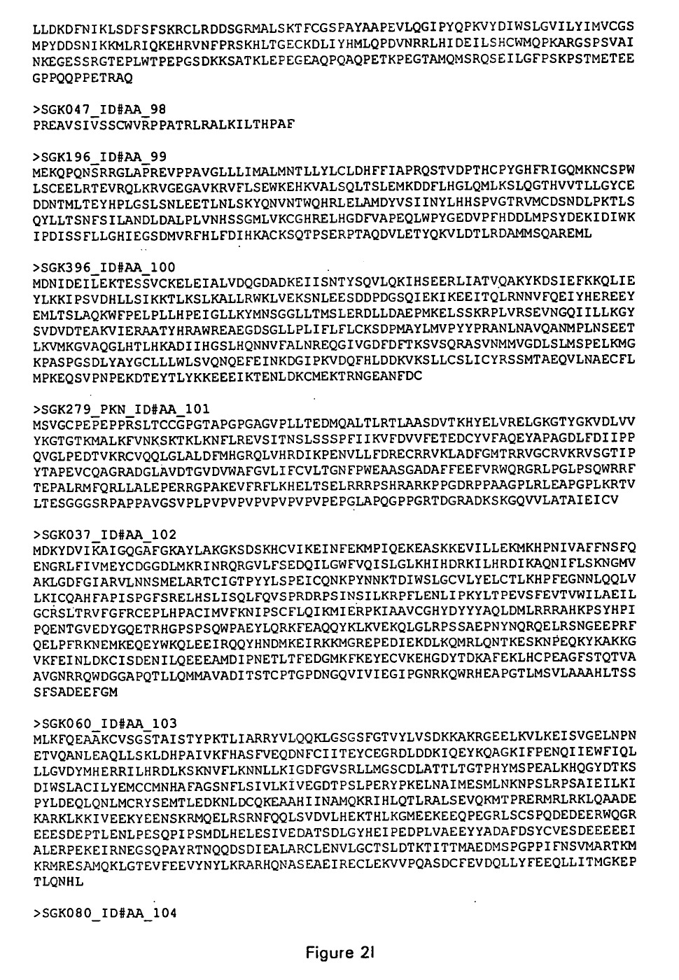

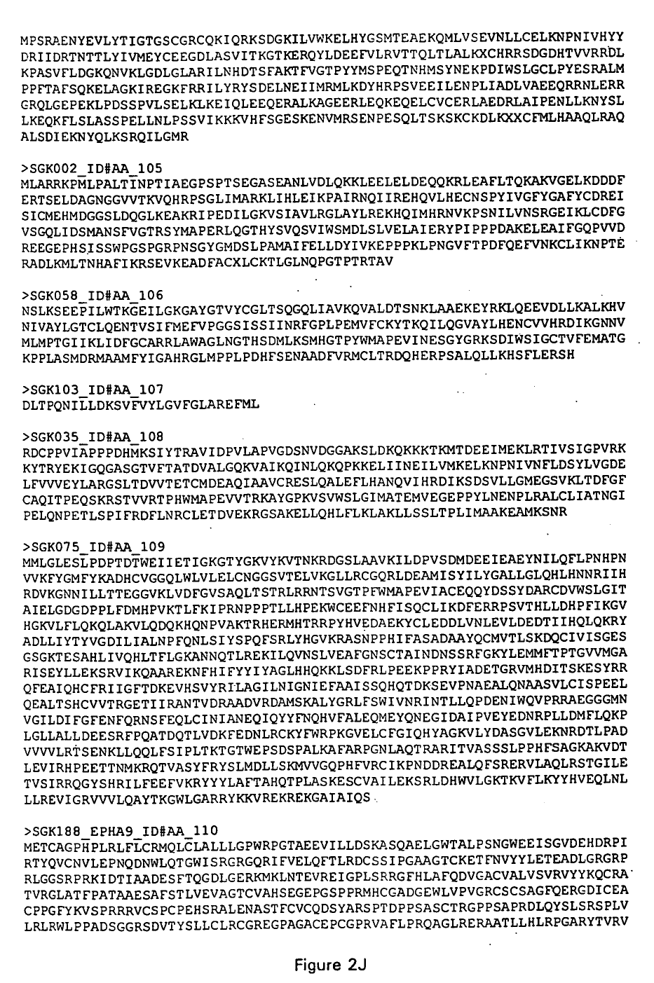

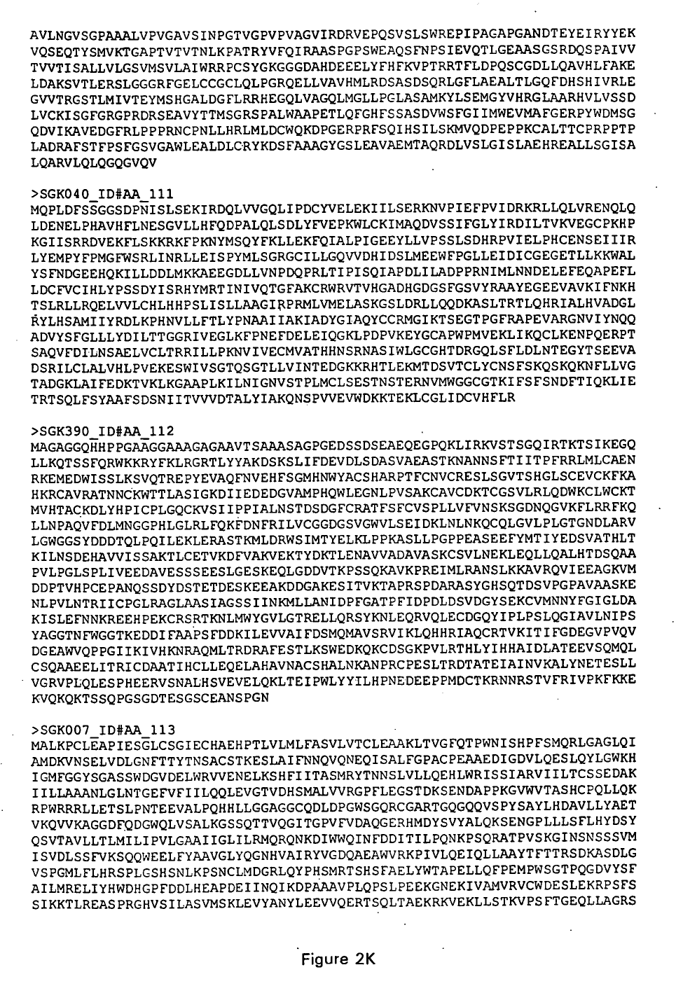

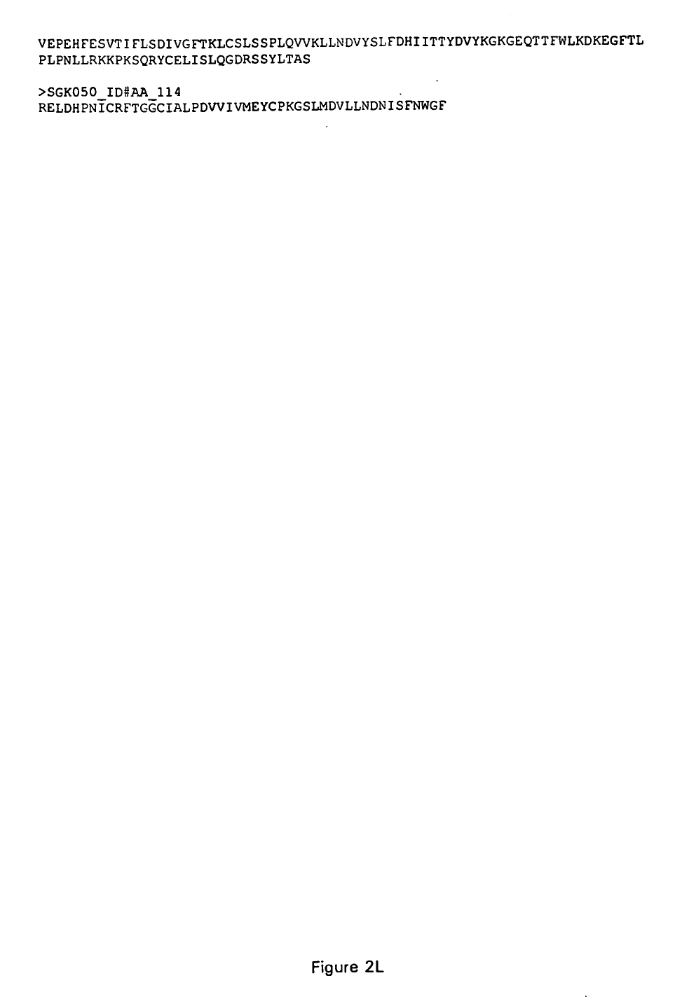

### DETAILED DESCRIPTION OF THE INVENTION

The invention provides, inter alia, protein kinase and kinase-like genes, as well as fragments thereof, which have been identified in genomic databases. In part, the invention provides nucleic acid molecules that are capable of encoding polypeptides having a kinase or kinase-like activity. By reference to Tables 1 though 8, below, genes of the invention can be better understood. The invention additionally provides a number of different embodiments, such as those described below.

Nucleic Acids

Associations of chromosomal localizations for mapped genes with amplicons implicated in cancer are based on literature searches (PubMed http://www.ncbi.nlm.nih.gov/entrez/query.fcgi), OMIM searches (Online Mendelian Inheritance in Man, http://www.ncbi.nlm.nih.gov/Omim/searchomim.html) and the comprehensive database of cancer amplicons maintained by Knuutila, et al. (Knuutila, et al., DNA copy number amplifications in human neoplasms. Review of comparative genomic hybridization studies. Am J Pathol 152:1107-1123, 1998. http://www.helsinki.fi/˜lgl_www/CMG.html). For many of the mapped genes, the cytogenetic region from Knuutila is listed followed by the number of cases with documented amplification and the total number of cases studied. Thus for SGK187, the entry “non-small cell lung cancer (12q24.1-24.3; 2/50)” means that the chromosomal position has been associated with non-small cell lung cancer, at position 12q24.1-24.3, which encompasses the SGK087's position, and the amplification has been noted in 2 of the 50 samples studied.

For single nucleotide polymorphisms, an accession number (for example, ss1581624 for SGK187) is given if the SNP is documented in dbSNP (the database of single nucleotide polymorphisms) maintained at NCBI (http://www.ncbi.nlm.nih.gov/SNP/index.html). The accession number for SNP can be used to retrieve the full SNP-containing sequence from this site. Candidate SNPs without a dbSNP accession number were identified by inspection of Blastn outputs of the patent sequences vs cDNA and genomic databases as indicated, for example, in Tables 9 and 10, provided in Example 1.

Nucleic Acid Probes, Methods, and Kits for Detection of Kinases

The invention additionally provides nucleic acid probes and uses therefor. A nucleic acid probe of the present invention may be used to probe an appropriate chromosomal or cDNA library by usual hybridization methods to obtain other nucleic acid molecules of the present invention. A chromosomal DNA or cDNA library may be prepared from appropriate cells according to recognized methods in the art (cf. “Molecular Cloning: A Laboratory Manual”, second edition, Cold Spring Harbor Laboratory, Sambrook, Fritsch, & Maniatis, eds., 1989).

In the alternative, chemical synthesis can be carried out in order to obtain nucleic acid probes having nucleotide sequences which correspond to N-terminal and C-terminal portions of the amino acid sequence of the polypeptide of interest. The synthesized nucleic acid probes may be used as primers in a polymerase chain reaction (PCR) carried out in accordance with recognized PCR techniques, essentially according to PCR Protocols, “A Guide to Methods and Applications”, Academic Press, Michael, et al., eds., 1990, utilizing the appropriate chromosomal or cDNA library to obtain the fragment of the present invention.

One skilled in the art can readily design such probes based on the sequence disclosed herein using methods of computer alignment and sequence analysis known in the art (“Molecular Cloning: A Laboratory Manual”, 1989, supra). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

One method of detecting the presence of nucleic acids of the invention in a sample comprises (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of nucleic acids of the invention in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or steptavidin). Preferably, the kit further comprises instructions for use.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

Categorization of the Polypeptides According to the Invention

For a number of protein kinases of the invention, there is provided a classification of the protein class and family to which it belongs, a summary of non-catalytic protein motifs, as well as a chromosomal location. This information is useful in determining function, regulation and/or therapeutic utility for each of the proteins. Amplification of chromosomal region can be associated with various cancers. For amplicons discussed in this application, the source of information was Knuutila, et al (Knuutila S, Björkqvist A-M, Autio K, Tarkkanen M, Wolf M, Monni O, Szymanska J, Larramendy M L, Tapper J, Pere H, El-Rifai W, Hemmer S, Wasenius V-M, Vidgren V & Zhu Y: DNA copy number amplifications in human neoplasms. Review of comparative genomic hybridization studies. Am J Pathol 152:1107-1123, 1998. http://www.helsinki.fi/˜lgl_www/CMG.html).

The kinase classification and protein domains often reflect pathways, cellular roles, or mechanisms of up- or down-stream regulation. Also disease-relevant genes often occur in families of related genes. For example, if one member of a kinase family functions as an oncogene, a tumor suppressor, or has been found to be disrupted in an immune, neurologic, cardiovascular, or metabolic disorder, frequently other family members may play a related role.

The expression analysis organizes kinases into groups that are transcriptionally upregulated in tumors and those that are more restricted to specific tumor types such as melanoma or prostate. This analysis also identifies genes that are regulated in a cell cycle dependent manner, and are therefore likely to be involved in maintaining cell cycle checkpoints, entry, progression, or exit from mitosis, oversee DNA repair, or are involved in cell proliferation and genome stability. Expression data also can identify genes expressed in endothelial sources or other tissues that suggest a role in angiogenesis, thereby implicating them as targets for control of diseases that have an angiogenic component, such as cancer, endometriosis, retinopathy and macular degeneration, and various ischemic or vascular pathologies. A proteins' role in cell survival can also be suggested based on restricted expression in cells subjected to external stress such as oxidative damage, hypoxia, drugs such as cisplatinum, or irradiation. Metastases-associated genes can be implicated when expression is restricted to invading regions of a tumor, or is only seen in local or distant metastases compared to the primary tumor, or when a gene is upregulated during cell culture models of invasion, migration, or motility.

Chromosomal location can identify candidate targets for a tumor amplicon or a tumor-suppressor locus. Summaries of prevalent tumor amplicons are available in the literature, and can identify tumor types to experimentally be confirmed to contain amplified copies of a kinase gene which localizes to an adjacent region.

As described herein, the polypeptides of the present invention can be classified, for example, among ten different groups. The salient features related to the biological and clinical implications of these different groups are described hereafter in more general terms.

A more specific characterization of the polypeptides of the invention, including potential biological and clinical implications, is provided, e.g., in EXAMPLES 2a and 2b.

### Classification of Polypeptides Exhibiting Kinase Activity

The following information also is referenced, for example, at Tables 1 and 2.

AGC Group

Family members are described that belong to the AGC group of protein kinases. The AGC group of protein kinases includes as its major prototypes protein kinase C (PKC), cAMP-dependent protein kinases (PKA), the G protein-coupled receptor kinases (ARK and rhodopsin kinase (GRK1)) as well as p70S6K and AKT.

Potential biological and clinical implications of the novel AGC group protein kinases are described in Example 2e. Novel AGC group kinases include: SEQ ID NO:58, SEQ ID NO:59, SEQ ID NO:60, SEQ ID NO:61, SEQ ID NO:62, SEQ ID NO:63, and SEQ ID NO:64.

CAMK Group

Family members are described that belong to the CAMK group of protein kinases. The CAMK group of protein kinases includes as its major prototypes the calmodulin-dependent protein kinases, elongation factor-2 kinases, phosphorylase kinase and the Snf1 and cAMP-dependent family of protein kinases.

Potential biological and clinical implications of the novel CAMK group of protein kinases are described in Example 2e. Novel CAMK group of protein kinases include: SEQ ID NO:65, SEQ ID NO:66, SEQ ID NO:67, SEQ ID NO:68, SEQ ID NO:69, SEQ ID NO:75, SEQ ID NO:76, SEQ ID NO:77, and SEQ ID NO:78.

Casein Kinase Group

Family members are described that belong to the casein kinase (CKI) group of protein kinases. The Casein kinase (CK) group of protein kinases includes as its major prototypes casein kinaseI (CKI) and casein kinaseII (CKII). Both CKI and CKII are ubiquitous, constitutively-active, second-messenger-independent kinases, highly conserved enzymes that exist in multiple isoforms. CKI functions in vesicular trafficking, DNA repair, cell cycle progression and cytokinesis (Cell Signal 1998 Nov. 10(10):699-71 1). CK2 functions in cell cycle progression in non-neural cells. CK2 has also been implicated in multiple signaling pathways in normal and disease states of the mammalian nervous systems (Prog Neurobiol February 2000; 60(3):211-46 ).

Potential biological and clinical implications of the novel casein kinase group of protein kinases are described in Example 2e. Novel casein kinase protein kinases include: SEQ ID NO: 79, and SEQ ID NO:80.

CMGC Group

Family members are described that belong to the cyclin-dependent kinase (CDK) group of protein kinases. The CMGC group of protein kinases includes as its major prototypes the cyclin-dependent protein kinases, as well as the MAPK kinases family, the GSK family and the CLK family of kinases.

Potential biological and clinical implications of the novel CMGC group of protein kinases are described in Example 2e. Novel CMGC protein kinases include: SEQ ID NO:81, SEQ ID NO:82, and SEQ ID NO: 83.

Microbial PK Group

Family members are described that belong to the microbial group of protein kinases. This group is defined, for example, by the protein kinases that include ABC1, RI01, YGR262, all of which have been initially identified from microbial genome sequencing projects (Proc Natl Acad Sci U S A 1999 Nov. 23;96(24):13603-10).

Potential biological and clinical implications of the novel microbial group of protein kinases are described in Example 2e. Novel microbial protein kinases include SEQ ID NO:84, SEQ ID NO:85, and SEQ ID NO:86.

“Other” Group

Family members are described that belong to the “Other” group of protein kinases. Within this group of protein kinases are members that have recognizable catalytic motifs that are identifiable by a hidden Markov model analysis, but fail to cluster with other protein kinases on the basis of their amino acid sequence homology over the catalytic region.

Potential biological and clinical implications of the novel protein kinases belonging to the Other group are described in Example 2e. Novel “Other” protein kinases include: SEQ ID NO: 87, SEQ ID NO: 88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, and SEQ ID NO:101.

The STE Group

Family members are described that belong to the STE group of protein kinases. The STE group of protein kinases includes, as its major prototypes, the NEK kinases, as well as the STE11 and STE20 family of sterile protein kinases.

Potential biological and clinical implications of the novel protein kinases belonging to the STE group are described in Example 2e. Novel STE protein kinases include: SEQ ID NO:102, SEQ ID NO:103, SEQ ID NO:104, SEQ ID NO:105, SEQ ID NO:106, SEQ ID NO:107, SEQ ID NO:108, and SEQ ID NO:109.

The TK Group

Family members are described that belong to the tyrosine kinase (TK) group of protein kinases. The TK group of protein kinases includes as its major prototypes the cytoplasmic and receptor families of protein kinases.

One family within this group of kinases is the “Eph” family. The Eph family, which is the largest sub-family of receptor tyrosine kinases in the human genome, has a stereotyped structure consisting of an N-terminal globular domain involved in ligand binding, two Type III fibronectin-like domains which contribute to receptor dimerization, a transmembrane domain, and an intracellular tyrosine kinase domain. The Eph family is composed of two subfamilies: the EphA receptors which generally bind to members of the GPI-linked Ephrin A family of ligands, and the EphB receptors which generally bind to the transmembrane Ephrin B family of ligands. Based on sequence similarity comparisons, EphA9—to which the polypeptide represented by SEQ ID NO:110 belongs, is a member of the EphA subfamily of receptors.

Investigation of the Eph family of receptors indicate their involvement in a wide variety of cellular processes. Activation of Eph receptors can lead to changes in intracellular signaling, cell adhesion, cytoskeleton effects, and synaptic remodeling. These Eph-dependent cellular effects in turn contribute to changes in tissue functions such as border formation, pattern formation, cell migration, neurogenesis, angiogenesis, and long term potentiation, among others. As a member of the Eph family, we expect that EphA9 will be involved in many of these functions as well.

Expression data for EphA9 indicate that it is expressed most prominently in the human central nervous system, the digestive system (especially in the colon and rectum) and the testes. Therefore, EphA9 may be involved in organization and function of the digestive tract, including the colon, and could contribute colorectal tumorigenesis and other disorders of the digestive tract.

EphA9 found in the nervous system could be involved in synaptogenesis, neuronal development and regeneration, axon outgrowth, and synaptic transmission. Therefore EphA9 might be important in neuronal survival and regeneration after injury, in long-term potentiation and memory formation, and in disorders of synaptic transmission such as epilepsy, depression, Parkinson's disease, and Alzheimer's disease.

Several Eph family receptors previously have been shown to be critical to several aspects of angiogenesis, such as remodeling, branching, sprouting and pruning of new blood vessels. EphA9, as an additional member of this family, also may be critical for aspects of angiogenesis. Thus, EphA9 may be relevant for a number diseases, including solid tumors, rheumatoid arthritis, and cardiovascular diseases.

Potential biological and clinical implications of the novel protein kinases belonging to the TK group are described in Example 2e. Novel TK protein kinases include: SEQ ID NO:100, and SEQ ID NO:111.

### Classification of Polypeptides Exhibiting Kinase-Like Activity

Two new family members are described that belong to the protein kinase (PK)-like insert “super family” of protein kinases. The PK-like superfamily of protein kinases includes the diacyl glycerol kinases (DGK) and the guanylate cyclases (GCyc), as decribed in the EXAMPLES.

Guanylate Cyclases (GCyc) Group

Guanylate cyclases are ubiquitous enzymes that convert GTP to cGMP and exist as membrane-bound and soluble isoforms. A diverse range of agonist that include peptide hormones, bacterial toxins as well as intracellular molecules such as calcium and cAMP regulate the enzymatic activity of guanylate cyclases. Stimulation of guanylate kinases modulates multiple downstream enzymes including cGMP-dependent protein kinases, cGMP-regulated phosphodiesterases, and cyclic nucleotide-gated ion channels. The modulation of cGMP levels by guanylate cyclases contributes to the regulation of vascular smooth muscle motility, intestinal fluid and electrolyte homeostasis, and retinal phototransduction (Pharmacol Rev September 2000; 52(3):375-414). As potential novel members of the guanylate cyclase family, disruptions in the signaling pathways in which SGK007 and SGK050 participate may alter cGMP homeostasis with pathophysiological implications.

Diacyl Glycerol Kinase (DGK) Group

A diacyl glycerol kinase phosphorylates the second messenger molecule diacyl glycerol leading to the formation of phosphatidic acid. Nine mammalain DGK isozymes have been described. The catalytic domain of a DGK usually is flanked by protein-protein interaction domains such as zinc fingers, pleckstrin homology domains and ankyrin repeats, as well as calcium-binding EF-hand structures. DGK's can be associated with the plasma membrane, nucleus and cytoskeleton. Experimental evidence supports the proposition that DGK's are translocated to and from these cellular compartments in response to agonists. At these intracellular locations, DGK's are able to modulate lipid metabolism and PKC activation, thereby triggering effector functions related to cell cycle progresion and differentiation (Int. J. Biochem. Cell Biol. 1997, (10):1139-43, J. Biol. Chem. 1999, 274(17):11447-50.)

SGK093—The Wnk Family of Serine/Threonine Kinases

Wnk3 is a member of a subfamily of serine/threonine kinases which includes a described prototype, Wnk1, isolated from rat. This family is characterized by an N-terminal catalytic domain with several unique sequence features, most notably a change of the invariant lysine in kinase subdomain II to a cysteine, coupled with a change of the third conserved glycine residue in subdomain I into a lysine. The resulting enzyme appears to maintain catalytic activity due to this concomitant switch. Wnk3 conserves both of these catalytic changes and therefore is predicted to maintain catalytic activity. The long C-terminal portion of the wnks includes many protein interaction domains such as SH3 binding sites and coiled coil regions.

The wnk family catalytic domain shows the highest similarity to two families of serine/threonine kinases: The MEKK-like kinases and the Ste20-like kinases. Both of these families can regulate enzymes in various MAPK signaling cascades, which are critical for many cellular processes such as mitogenesis, differentiation, cell survival, and stress response. The Ste20 kinases are also involved in regulation of the ras/rac/rho/cdc42 pathways and subsequent downstream effects on cytoskeleton.

Wnk3 shows high expression in human kidney, in kidney carcinoma cell lines, in prostate, prostate cell lines, and prostate tumor bone metastases, in colorectal tissue and tumor cell lines, and in human leukemia cells. Therefore wnk3 may be involved in the normal homeostasis and functioning of the human kidney, prostate, and digestive system, and may be involved in tumorigenesis which arises from these three tissues. High expression in human leukemia cell lines indicates a possible role in the development of that disease as well.

### Therapeutic Methods According to the Invention

Diagnostics:

The invention provides methods for detecting a polypeptide in a sample as a diagnostic tool for diseases or disorders, wherein the method comprises the steps of: (a) contacting the sample with a nucleic acid probe which hybridizes under hybridization assay conditions to a nucleic acid target region of a polypeptide selected from the group consisting of SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, metabolic disorder including diabetes, reproductive disorders including infertility, and cancer.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined supra.

The diseases for which detection of genes in a sample could be diagnostic include diseases in which nucleic acid (DNA and/or RNA) is amplified in comparison to normal cells. By “amplification” is meant increased numbers of DNA or RNA in a cell compared with normal cells.

“Amplification” as it refers to RNA can be the detectable presence of RNA in cells, since in some normal cells there is no basal expression of RNA. In other normal cells, a basal level of expression exists, therefore in these cases amplification is the detection of at least 1-2-fold, and preferably more, compared to the basal level.

The diseases that could be diagnosed by detection of nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Antibodies, Hybridomas, Methods of Use and Kits for Detection of Kinases

The present invention relates to an antibody having binding affinity to a kinase of the invention. The polypeptide may have the amino acid sequence selected from the group consisting of those set forth in SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107,SEQ ID NO:108, SEQ ID NO:109, SEQ ID NO:110, SEQ ID NO:111, SEQ ID NO:112, SEQ ID NO:113, and SEQ ID NO:114, or a functional derivative thereof, or at least 9 contiguous amino acids thereof (preferably, at least 20, 30, 35, or 40 contiguous amino acids thereof).

The present invention also relates to an antibody having specific binding affinity to a kinase of the invention. Such an antibody may be isolated by comparing its binding affinity to a kinase of the invention with its binding affinity to other polypeptides. Those which bind selectively to a kinase of the invention would be chosen for use in methods requiring a distinction between a kinase of the invention and other polypeptides. Such methods could include, but should not be limited to, the analysis of altered kinase expression in tissue containing other polypeptides.

The kinases of the present invention can be used in a variety of procedures and methods, such as for the generation of antibodies, for use in identifying pharmaceutical compositions, and for studying DNA/protein interaction.

The kinases of the present invention can be used to produce antibodies or hybridomas. One skilled in the art will recognize that if an antibody is desired, such a peptide could be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting.

The present invention also relates to a hybridoma which produces the above-described monoclonal antibody, or binding fragment thereof. A hybridoma is an immortalized cell line which is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Agl4 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art, for example, see Stemberger et al., J. Histochem. Cytochem. 18:315, 1970; Bayer et al., Meth. Enzym. 62:308, 1979; Engval et al., Immunol. 109:129, 1972; Goding, J. Immunol. Meth. 13:215, 1976. The labeled antibodies of the present invention can be used for in vitro, in vivo, and in situ assays to identify cells or tissues which express a specific peptide.

The above-described antibodies may also be immobilized on a solid support. Examples of such solid supports include plastics such as polycarbonate, complex carbohydrates such as agarose and sepharose, acrylic resins such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The immobilized antibodies of the present invention can be used for in vitro, in vivo, and in situ assays as well as in immunochromotography.

Furthermore, one skilled in the art can readily adapt currently available procedures, as well as the techniques, methods and kits disclosed herein with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W.H. Freeman, NY, pp. 289-307, 1992; Kaspczak et al., Biochemistry 28:9230-9238, 1989).

Anti-peptide peptides can be generated by replacing the basic amino acid residues found in the peptide sequences of the kinases of the invention with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

The present invention also encompasses a method of detecting a kinase polypeptide in a sample, comprising: (a) contacting the sample with an above-described antibody, under conditions such that immunocomplexes form, and (b) detecting the presence of said antibody bound to the polypeptide. In detail, the methods comprise incubating a test sample with one or more of the antibodies of the present invention and assaying whether the antibody binds to the test sample. Altered levels of a kinase of the invention in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion-based Ouchterlony, or rocket immunofluorescent assays) can readily be adapted to employ the antibodies of the present invention. Examples of such assays can be found in Chard (“An Introduction to Radioimmunoassay and Related Techniques” Elsevier Science Publishers, Amsterdam, The Netherlands, 1986), Bullock et al. (“Techniques in Immunocytochemistry,” Academic Press, Orlando, Fla. Vol. 1, 1982; Vol. 2, 1983; Vol. 3, 1985), Tijssen (“Practice and Theory of Enzyme Immunoassays: Laboratory Techniques in Biochemistry and Molecular Biology,” Elsevier Science Publishers, Amsterdam, The Netherlands, 1985).

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed. Methods for preparing protein extracts or membrane extracts of cells are well known in the art and can readily be adapted in order to obtain a sample which is testable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

Isolation of Compounds Capable of Interacting with Kinases

The present invention also relates to a method of detecting a compound capable of binding to a kinase of the invention comprising incubating the compound with a kinase of the invention and detecting the presence of the compound bound to the kinase. The compound may be present within a complex mixture, for example, serum, body fluid, or cell extracts.

The present invention also relates to a method of detecting an agonist or antagonist of kinase activity or kinase binding partner activity comprising incubating cells that produce a kinase of the invention in the presence of a compound and detecting changes in the level of kinase activity or kinase binding partner activity. The compounds thus identified would produce a change in activity indicative of the presence of the compound. The compound may be present within a complex mixture, for example, serum, body fluid, or cell extracts. Once the compound is identified it can be isolated using techniques well known in the art.

Modulating Polypeptide Activity

The invention additionally provides methods for treating a disease or abnormal condition by administering to a patient in need of such treatment a substance that modulates the activity of a polypeptide selected from the group consisting of SEQ ID NO:58, SEQ ID NO:59, SEQ ID NO:60, SEQ ID NO:61, SEQ ID NO:62, SEQ ID NO:63, SEQ ID NO: 64, SEQ ID NO: 65, SEQ ID NO: 66, SEQ ID NO: 67, SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, and SEQ ID NO:114. Preferably, the disease is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, metabolic and reproductive disorders, and cancer.

Substances useful for treatment of disorders or diseases preferably show positive results in one or more assays for an activity corresponding to treatment of the disease or disorder in question Substances that modulate the activity of the polypeptides preferably include, but are not limited to, antisense oligonucleotides and inhibitors of protein kinases.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth, and/or differentiation of cells; (b) inhibition (slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation or cell survival. An abnormal condition may also include irregularities in cell cycle progression, i.e., irregularities in normal cell cycle progression through mitosis and meiosis.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to, neurodegenerative disorders, slow wound healing rates, and slow tissue grafting healing rates.

Abnormal cell survival conditions may also relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “aberration”, in conjunction with the function of a kinase in a signal transduction process, refers to a kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig or goat, more preferably a monkey or ape, and most preferably a human.

The present invention also encompasses a method of agonizing (stimulating) or antagonizing kinase associated activity in a mammal comprising administering to said mammal an agonist or antagonist to a kinase of the invention in an amount sufficient to effect said agonism or antagonism. A method of treating diseases in a mammal with an agonist or antagonist of the activity of one of the kinases of the invention comprising administering the agonist or antagonist to a mammal in an amount sufficient to agonize or antagonize kinase-associated functions is also encompassed in the present application.

In an effort to discover novel treatments for diseases, biomedical researchers and chemists have designed, synthesized, and tested molecules that inhibit the function of protein kinases. Some small organic molecules form a class of compounds that modulate the function of protein kinases. Examples of molecules that have been reported to inhibit the function of protein kinases include, but are not limited to, bis monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al).

Compounds that can traverse cell membranes and are resistant to acid hydrolysis are potentially advantageous as therapeutics as they can become highly bioavailable after being administered orally to patients. However, many of these protein kinase inhibitors only weakly inhibit the function of protein kinases. In addition, many inhibit a variety of protein kinases and will therefore cause multiple side-effects as therapeutics for diseases.

Some indolinone compounds, however, form classes of acid resistant and membrane permeable organic molecules. WO 96/22976 (published Aug. 1, 1996 by Ballinari et al.) describes hydrosoluble indolinone compounds that harbor tetralin, naphthalene, quinoline, and indole substituents fused to the oxindole ring. These bicyclic substituents are in turn substituted with polar moieties including hydroxylated alkyl, phosphate, and ether moieties. U.S. patent application Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187) and Ser. No. 08/485,323, filed Jun. 7, 1995, entitled “Benzylidene-Z-Indoline Compounds for the Treatment of Disease” by Tang et al. (Lyon & Lyon Docket No. 223/298) and International Patent Publications WO 96/40116, published Dec. 19, 1996 by Tang, et al., and WO 96/22976, published Aug. 1, 1996 by Ballinari et al., all of which are incorporated herein by reference in their entirety, including any drawings, figures, or tables, describe indolinone chemical libraries of indolinone compounds harboring other bicyclic moieties as well as monocyclic moieties fused to the oxindole ring. Application Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187), Ser. No. 08/485,323, filed Jun. 7, 1995, entitled “Benzylidene-Z-Indoline Compounds for the Treatment of Disease” by Tang et al. (Lyon & Lyon Docket No. 223/298), and WO 96/22976, published Aug. 1, 1996 by Ballinari et al. teach methods of indolinone synthesis, methods of testing the biological activity of indolinone compounds in cells, and inhibition patterns of indolinone derivatives.

Other examples of substances capable of modulating kinase activity include, but are not limited to, tyrphostins, quinazolines, quinoxolines, and quinolines. The quinazolines, tyrphostins, quinolines, and quinoxolines referred to above include well known compounds such as those described in the literature. For example, representative publications describing quinazolines include Barker et al., EPO Publication No. 0 520 722 A1; Jones et al., U.S. Pat. No. 4,447,608; Kabbe et al., U.S. Pat. No. 4,757,072; Kaul and Vougioukas, U.S. Pat. No. 5,316,553; Kreighbaum and Comer, U.S. Pat. No. 4,343,940; Pegg and Wardleworth, EPO Publication No. 0 562 734 A1; Barker et al., (1991) Proc. of Am. Assoc. for Cancer Research 32:327; Bertino, J. R., (1979) Cancer Research 3:293-304; Bertino, J. R., (1979) Cancer Research 9(2 part 1):293-304; Curtin et al., (1986) Br. J. Cancer 53:361-368; Fernandes et al., (1983) Cancer Research 43:1117-1123 ; Ferris et al. J. Org. Chem. 44(2):173-178; Fry et al., (1994) Science 265:1093-1095; Jackman et al., (1981) Cancer Research 51:5579-5586; Jones et al. J. Med. Chem. 29(6):1114-1118; Lee and Skibo, (1987) Biochemistry 26(23):7355-7362; Lemus et al., (1989) J. Org. Chem. 54:3511-3518; Ley and Seng, (1975) Synthesis 1975:415-522; Maxwell et al., (1991) Magnetic Resonance in Medicine 17:189-196 ; Mini et al., (1985) Cancer Research 45:325-330; Phillips and Castle, J. (1980) Heterocyclic Chem. 17(19):1489-1596; Reece et al., (1977) Cancer Research 47(11):2996-2999; Sculier et al., (1986) Cancer Immunol. and Immunother. 23, A65; Sikora et al., (1984) Cancer Letters 23:289-295; Sikora et al., (1988) Analytical Biochem. 172:344-355; all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., (1994) J. Med. Chem. 37:2627-2629; MaGuire, J. (1994) Med Chem. 37:2129-2131; Burke et al., (1993) J. Med. Chem. 36:425-432; and Burke et al. (1992) BioOrganic Med. Chem. Letters 2:1771-1774, all of which are incorporated by reference in their entirety, including any drawings.

Tyrphostins are described in Allen et al., (1993) Clin. Exp. Immunol. 91:141-156; Anafi et al., (1993) Blood 82:12, 3524-3529; Baker et al., (1992) J. Cell Sci. 102:543-555; Bilder et al., (1991) Amer. Physiol. Soc. pp. 6363-6143:C721-C730; Brunton et al., (1992) Proceedings of Amer. Assoc. Cancer Rsch. 33:558; Bryckaert et al., (1992) Exp. Cell Research 199:255-261; Dong et al., (1993) J. Leukocyte Biology 53:53-60; Dong et al., (1993) J. Immunol. 151(5):2717-2724; Gazit et al., (1989) J. Med. Chem. 32, 2344-2352; Gazit et al., (1993) J. Med. Chem. 36:3556-3564; Kaur et al., (1994) Anti-Cancer Drugs 5:213-222; King et al., (1991) Biochem. J. 275:413-418; Kuo et al., (1993) Cancer Letters 74:197-202; Levitzki, A., (1992) The FASEB J. 6:3275-3282; Lyall et al., (1989) J. Biol. Chem. 264:14503-14509; Peterson et al., (1993) The Prostate 22:335-345; Pillemer et al., (1992) Int. J. Cancer 50:80-85; Posner et al., (1993) Molecular Pharmacology 45:673-683; Rendu et al., (1992) Biol. Pharmacology 44(5):881-888; Sauro and Thomas, (1993) Life Sciences 53:371-376; Sauro and Thomas, (1993) J. Pharm. and Experimental Therapeutics 267(3):119-1125; Wolbring et al., (1994) J. Biol. Chem. 269(36):22470-22472; and Yoneda et al., (1991) Cancer Research 51:4430-4435; all of which are incorporated herein by reference in their entirety, including any drawings.

Other compounds that could be used as modulators include oxindolinones such as those described in U.S. patent application Ser. No. 08/702,232 filed Aug. 23, 1996, incorporated herein by reference in its entirety, including any drawings.

### Recombinant DNA Technology

DNA Constructs Comprising a Kinase Nucleic Acid Molecule and Cells Containing These Constructs:

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecule. The present invention also relates to a nucleic acid molecule comprising a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and thereby is capable of expressing a polypeptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a kinase of the invention may be obtained by the above-described methods. This region may be retained for its transcriptional termination regulatory sequences, such as termination and polyadenylation. Thus, by retaining the 3′-region naturally contiguous to the DNA sequence encoding a kinase of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a kinase of the invention) are said to be operably linked if the nature of the linkage between the two DNA sequences does not (1) result in the introduction of a frame-shift mutation, (2) interfere with the ability of the promoter region sequence to direct the transcription of a gene sequence encoding a kinase of the invention, or (3) interfere with the ability of the gene sequence of a kinase of the invention to be transcribed by the promoter region sequence. Thus, a promoter region would be operably linked to a DNA sequence if the promoter were capable of effecting transcription of that DNA sequence. Thus, to express a gene encoding a kinase of the invention, transcriptional and translational signals recognized by an appropriate host are necessary.

The present invention encompasses the expression of a gene encoding a kinase of the invention (or a functional derivative thereof) in either prokaryotic or eukaryotic cells. Prokaryotic hosts are, generally, very efficient and convenient for the production of recombinant proteins and are, therefore, one type of preferred expression system for kinases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include λgt10, λgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a kinase of the invention (or a functional derivative thereof) in a prokaryotic cell, it is necessary to operably link the sequence encoding the kinase of the invention to a functional prokaryotic promoter. Such promoters may be either constitutive or, more preferably, regulatable (i.e., inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, λrecA, acZ, λacI, and gal promoters of E. coli, the α-amylase (Ulmanen et al., J. Bacteriol. 162:176-182, 1985) and the ç-28-specific promoters of B. subtilis (Gilman et al., Gene Sequence 32:11-20, 1984), the promoters of the bacteriophages of Bacillus (Gryczan, in: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the kinase polypeptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

In addition, plant cells are also available as hosts, and control sequences compatible with plant cells are available, such as the cauliflower mosaic virus 35S and 19S, and nopaline synthase promoter and polyadenylation signal sequences. Another preferred host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of kinases of the invention in insect cells (Jasny, Science 238:1653, 1987; Miller et al., in: Genetic Engineering, Vol. 8, Plenum, Setlow et al., eds., pp. 277-297, 1986).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e., pre-peptides). Several possible vector systems are available for the expression of kinases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of kinases of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it is preferable to ensure that the linkage between a eukaryotic promoter and a DNA sequence which encodes a kinase of the invention (or a functional derivative thereof) does not contain any intervening codons which are capable of encoding a methionine (i.e., AUG). The presence of such codons results either in the formation of a fusion protein (if the AUG codon is in the same reading frame as the kinase of the invention coding sequence) or a frame-shift mutation (if the AUG codon is not in the same reading frame as the kinase of the invention coding sequence).

A nucleic acid molecule encoding a kinase of the invention and an operably linked promoter may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280-289, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColE1, pSC101, pACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include plJ101 (Kendall et al., J. Bacteriol. 169:4177-4183, 1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a kinase of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Transgenic Animals:

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombina-tion (Capecchi, Science 244:1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al. (Nature 338:153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244:1281-1288, 1989; and Simms et al., Bio/Technology 6:179-183, 1988).

Thus, the invention provides transgenic, nonhuman mammals containing a transgene encoding a kinase of the invention or a gene affecting the expression of the kinase. Such transgenic nonhuman mammals are particularly useful as an in vivo test system for studying the effects of introduction of a kinase, or regulating the expression of a kinase (i.e., through the introduction of additional genes, antisense nucleic acids, or ribozymes).

A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode human kinases. Native expression in an animal may be reduced by providing an amount of antisense RNA or DNA effective to reduce expression of the receptor.

Gene Therapy:

Kinases or their genetic sequences will also be useful in gene therapy (reviewed in Miller, Nature 357:455-460, 1992). Miller states that advances have resulted in practical approaches to human gene therapy that have demonstrated positive initial results. The basic science of gene therapy is described in Mulligan (Science 260:926-931, 1993).

In one preferred embodiment, an expression vector containing a kinase coding sequence is inserted into cells, the cells are grown in vitro and then infused in large numbers into patients. In another preferred embodiment, a DNA segment containing a promoter of choice (for example a strong promoter) is transferred into cells containing an endogenous gene encoding kinases of the invention in such a manner that the promoter segment enhances expression of the endogenous kinase gene (for example, the promoter segment is transferred to the cell such that it becomes directly linked to the endogenous kinase gene).

The gene therapy may involve the use of an adenovirus containing kinase cDNA targeted to a tumor, systemic kinase increase by implantation of engineered cells, injection with kinase-encoding virus, or injection of naked kinase DNA into appropriate tissues.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction, may be used to inhibit an abnormal, deleterious signal transduction event.

Expression vectors derived from viruses such as retroviruses, vaccinia virus, adenovirus, adeno-associ-ated virus, herpes viruses, several RNA viruses, or bovine papilloma virus, may be used for delivery of nucleotide sequences (e.g., cDNA) encod-ing recom-binant kinase of the invention protein into the targeted cell population (e.g., tumor cells). Methods which are well known to those skilled in the art can be used to construct recombinant viral vectors contain-ing coding sequences (Maniatis et al., Molecular Cloning: A Laboratory Manual, Cold Spring Harbor Laboratory, N.Y., 1989; Ausubel et al., Current Proto-cols in Molecular Biology, Greene Publishing Associates and Wiley Interscience, N.Y., 1989). Alter-natively, recombinant nucleic acid mole-cules encoding protein sequences can be used as naked DNA or in a recon-stituted system e.g., lipo-somes or other lipid systems for delivery to target cells (e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins (Miller, supra).

In its simplest form, gene transfer can be performed by simply injecting minute amounts of DNA into the nucleus of a cell, through a process of microinjection (Capecchi, Cell 22:479-88, 1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with calcium phosphate and taken into cells by pinocytosis (Chen et al., Mol. Cell Biol. 7:2745-52, 1987); electroporation, wherein cells are exposed to large voltage pulses to introduce holes into the membrane (Chu et al., Nucleic Acids Res. 15:1311-26, 1987); lipofection/liposome fusion, wherein DNA is packaged into lipophilic vesicles which fuse with a target cell (Felgner et al., Proc. Natl. Acad. Sci. USA. 84:7413-7417, 1987); and particle bombardment using DNA bound to small projectiles (Yang et al., Proc. Natl. Acad. Sci. 87:9568-9572, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene (Curiel et al., Am. J. Respir. Cell. Mol. Biol., 6:247-52, 1992).

As used herein “gene transfer” means the process of introducing a foreign nucleic acid molecule into a cell. Gene transfer is commonly performed to enable the expres-sion of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cyto-plasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a kinase polypeptide is provided in which the nucleic acid sequence is expressed only in specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

### Pharmaceutical Formulations and Routes of Administration

The compounds described herein can be administered to a human patient per se, or in pharmaceutical compositions where it is mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., latest edition.

Routes of Administration:

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Composition/Formulation:

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the tyrosine or serine/threonine kinase modulating compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable Dosage Regimens:

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the tyrosine or serine/threonine kinase activity). Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

In another example, toxicity studies can be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia: 229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness.

Plasma levels should reflect the potency of the drug. Generally, the more potent the compound the lower the plasma levels necessary to achieve efficacy.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging:

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

### Functional Derivatives

Also provided herein are functional derivatives of a polypeptide or nucleic acid of the invention. By “functional derivative” is meant a “chemical derivative,” “fragment,” or “variant,” of the polypeptide or nucleic acid of the invention, which terms are defined below. A functional derivative retains at least a portion of the function of the protein, for example reactivity with an antibody specific for the protein, enzymatic activity or binding activity mediated through noncatalytic domains, which permits its utility in accordance with the present invention. It is well known in the art that due to the degeneracy of the genetic code numerous different nucleic acid sequences can code for the same amino acid sequence. Equally, it is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the genes of the invention could be synthesized to give a nucleic acid sequence significantly different from one selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57. The encoded amino acid sequence thereof would, however, be preserved. In addition, the nucleic acid sequence may comprise a nucleotide sequence which results from the addition, deletion or substitution of at least one nucleotide to the 5′-end and/or the 3′-end of the nucleic acid formula selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57, or a derivative thereof. Any nucleotide or polynucleotide may be used in this regard, provided that its addition, deletion or substitution does not alter the amino acid sequence of selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, and SEQ ID NO:57 which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the kinase genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

A “chemical derivative” of the complex contains additional chemical moieties not normally a part of the protein. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect or reversing the charge of the lysinyl residues. Other suitable reagents for derivatizing primary amine containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine alpha-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimide (R′—N—C—N—R′) such as 1-cyclohexyl-3-(2-morpholinyl(4-ethyl) carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking the component peptides of the protein to each other or to other proteins in a complex to a water-insoluble support matrix or to other macromolecular carriers. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl)dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W. H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half life, and the like. The moieties may alternatively eliminate or attenuate any undesirable side effect of the protein complex and the like. Moieties capable of mediating such effects are disclosed, for example, in Remington's Pharmaceutical Sciences, 18th ed., Mack Publishing Co., Easton, Pa. (1990).

The term “fragment” is used to indicate a polypeptide derived from the amino acid sequence of the proteins, of the complexes having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence. Fragments of a protein are useful for screening for substances that act to modulate signal transduction, as described herein. It is understood that such fragments may retain one or more characterizing portions of the native complex. Examples of such retained characteristics include: catalytic activity; substrate specificity; interaction with other molecules in the intact cell; regulatory functions; or binding with an antibody specific for the native complex, or an epitope thereof.

Another functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described above.

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183) wherein nucleotides in the DNA coding the sequence are modified such that a modified coding sequence is modified, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

### TABLES AND DESCRIPTION THEREOF

Table 1 documents the name of each gene, the classification of each gene, the positions of the open reading frames within the sequence, and the length of the corresponding peptide. From left to right the data presented is as follows: “Gene Name”, “ID#na”, “ID#aa”, “FL/Cat”, “Superfamily”, “Group”, “Family”, “NA_length”, “ORF Start”, “ORF End”, “ORF Length”, and “AA_length”. “Gene name” refers to name given the sequence encoding the kinase or kinase-like enzyme. Each gene is represented by “SGK” designation followed by a number. The SGK name usually represents multiple overlapping sequences built into a single contiguous sequence (a “contig”). The “ID#na” and “ID#aa” refer to the identification numbers given each nucleic acid and amino acid sequence in this patent. “FL/Cat” refers to the length of the gene, with FL indicating full length, and “Cat’ indicating that only the catalytic domain is presented. “Partial” in this column indicates that the sequence encodes a partial protein kinase catalytic domain. “Superfamily” identifies whether the gene is a protein kinase or protein-kinase-like. “Group” and “Family” refer to the protein kinase classification defined by sequence homology and based on previously established phylogenetic analysis [Hardie, G. and Hanks S. The Protein Kinase Book, Academic Press (1995) and Hunter T. and Plowman, G. Trends in Biochemical Sciences (1977) 22:18-22 and Plowman G. D. et al. (1999) Proc. Natl. Acad. Sci. 96:13603-13610)]. “NA_length” refers to the length in nucleotides of the corresponding nucleic acid sequence. “ORF start” refers to the beginning nucleotide of the open reading frame. “ORF end” refers to the last nucleotide of the open reading frame, excluding the stop codon. “ORF length” refers to the length in nucleotides of the open reading frame (excluding the stop codon): “AA length” refers to the length in amino acids of the peptide encoded in the corresponding nuclei acid sequence. (SNPs are respectively disclosued as SEQ ID NOS 272, 203, 206-210, 215-218, 220-222, 224-226, 229-236, 238, 273, 242-243, 245, 274, 246, 275, 251, 253, 256, 261, 276, 262-264 and 266-268).

TABLE 1
Open Reading Frames
413406_1.xls

| | | | | Super- | | | | | | | | | |
|---|---|---|---|---|---|---|---|---|---|---|---|---|---|
| Gene Name | ID#na | ID#aa | FL/Cat | family | Group | Family | NA_length | ORF Start | ORF End | ORF Length | AA_length | Genomic ID | sort1 |
| SGK187, CRIK | 1 | 58 | FL | PK | AGC | DMPK | 6159 | 1 | 6159 | 6159 | 2053 | 4926912_1_1 | 44 |
| SGK064, GRK7 | 2 | 59 | FL | PK | AGC | GRK | 1662 | 1 | 1659 | 1659 | 553 | 11000284009826 | 23 |
| SGK409, KIAA0303 | 3 | 60 | FL | PK | AGC | MAST | 7785 | 1 | 7569 | 7569 | 2523 | 6671897_1_1 | 54 |
| SGK021 | 4 | 61 | Cat | PK | AGC | Mo3C11.1_ce | 981 | 1 | 981 | 981 | 327 | 17000028181153 | 8 |
| SGK410 | 5 | 62 | FL | PK | AGC | PKC | 2260 | 72 | 1964 | 1893 | 631 | 7018620_2 | 55 |
| SGK069 | 6 | 63 | Cat | PK | AGC | Unique | 1716 | 1 | 1716 | 1716 | 572 | 11000283338966 | 26 |
| SGK110 | 7 | 64 | partial | PK | AGC | Unique | 1119 | 1 | 1119 | 1119 | 373 | 11000284702500 | 35 |
| SGK124 | 9 | 66 | FL | PK | CAMK | AMPK | 1077 | 1 | 1074 | 1074 | 358 | 17000047891899 | 37 |
| SGK254, CAMKKa | 10 | 67 | FL | PK | CAMK | CAMK | 1542 | 1 | 1539 | 1539 | 513 | 4454511_1_3 | 47 |
| SGK297, CaMKIb2 | 11 | 68 | FL | PK | CAMK | CAMK | 1032 | 1 | 1029 | 1029 | 343 | 17000113122540 | 50 |
| SGK411, CaMKII | 12 | 69 | FL | PK | CAMK | CAMK | 1500 | 1 | 1497 | 1497 | 499 | 2828765_1_3 | 56 |
| delta2 | | | | | | | | | | | | | |
| SGK027 | 13 | 70 | FL | PK | CAMK | EMK | 1311 | 1 | 1308 | 1308 | 436 | 17000029868654 | 9 |
| SGK046b | 14 | 71 | partial | PK | CAMK | EMK | 225 | 1 | 225 | 225 | 75 | 11000284253087 | 16 |
| SGK046c | 15 | 72 | partial | PK | CAMK | EMK | 117 | 1 | 117 | 117 | 39 | 11000284253087 | 17 |
| SGK089 | 16 | 73 | partial | PK | CAMK | EMK | 252 | 1 | 252 | 252 | 84 | 11000283986586 | 32 |
| SGK133 | 17 | 74 | FL | PK | CAMK | EMK | 2385 | 1 | 2382 | 2382 | 794 | 6671894_1_1 | 40 |
| SGK004, MSK | 18 | 75 | FL | PK | CAMK | EMK-1 | 2361 | 1 | 2358 | 2358 | 786 | 11000257744647 | 3 |
| SGK006 | 19 | 76 | Cat | PK | CAMK | EMK-1 | 789 | 1 | 789 | 789 | 263 | 11000257829454 | 4 |
| SGK180, SNRK | 20 | 77 | FL | PK | CAMK | EMK-1 | 2298 | 1 | 2295 | 2295 | 765 | 17000057577785 | 43 |
| SGK386, MLCKs | 21 | 78 | FL | PK | CAMK | MLCK | 1836 | 1 | 1836 | 1836 | 612 | 17000140438265 | 51 |
| SGK003 | 22 | 79 | FL | PK | CKI | CKI | 1014 | 1 | 1014 | 1014 | 337 | 11000257741254 | 2 |
| SGK066 | 23 | 80 | Cat | PK | CKI | CKI | 1200 | 1 | 1200 | 1200 | 400 | 11000283296349 | 24 |
| SGK041, NKIAMRE | 24 | 81 | FL | PK | CMGC | CDK | 1773 | 1 | 1773 | 1773 | 591 | 11000257953829 | 15 |
| SGK112 | 25 | 82 | FL | PK | CMGC | CDK | 1083 | 1 | 1080 | 1080 | 360 | 17000035915087 | 36 |
| SGK038, ERK7 | 26 | 83 | FL | PK | CMGC | MAPK | 1677 | 1 | 1674 | 1674 | 558 | 17000030278391 | 13 |
| SGK429 | 28 | 85 | FL | PK | Microbial PK | ABC1 | 1881 | 1 | 1878 | 1878 | 626 | 5001549_3 | 60 |
| SGK152, SUDD | 29 | 86 | FL | PK | Microbial PK | RI01 | 1560 | 1 | 1557 | 1557 | 519 | 11000258249295 | 41 |
| SGK077 | 30 | 87 | FL | PK | Other | C26C2_Ce | 2397 | 1 | 2394 | 2394 | 798 | 11000283672012 | 29 |
| SGK093, Wnk3 | 31 | 88 | FL | PK | Other | C26C2_Ce | 4542 | 1 | 4539 | 4539 | 1513 | 11000284063441 | 33 |
| SGK074 | 32 | 89 | Cat | PK | Other | DYRK | 1065 | 1 | 1065 | 1065 | 355 | 11000283476699 | 27 |
| SGK087 | 33 | 90 | FL | PK | Other | DYRK | 2112 | 1 | 2109 | 2109 | 703 | 17000036171996 | 31 |
| SGK295, KIS | 34 | 91 | FL | PK | Other | DYRK | 1260 | 1 | 1257 | 1257 | 419 | 17000113079883 | 49 |
| SGK419 | 35 | 92 | FL | PK | Other | NAK | 1986 | 1 | 1983 | 1983 | 661 | 5932598_100001_1 | 57 |
| SGK125, MYO3A | 36 | 93 | FL | PK | Other | NinaC | 4848 | 1 | 4845 | 4845 | 1615 | 17000078090910 | 38 |
| SGK445 | 37 | 94 | partial | PK | Other | PLK | 204 | 1 | 204 | 204 | 68 | 4000001803382 | 61 |
| SGK127 | 38 | 95 | FL | PK | Other | RAF | 2838 | 1 | 2835 | 2835 | 945 | 17000062804843 | 39 |
| SGK009, ANKRD3 | 39 | 96 | FL | PK | Other | RIP | 2499 | 1 | 2496 | 2496 | 832 | 11000258161587 | 6 |
| SGK421, STK22A, TSK1 | 40 | 97 | FL | PK | Other | STK22A | 1104 | 1 | 1101 | 1101 | 367 | 6759077_1_4 | 59 |
| SGK047 | 41 | 98 | partial | PK | Other | STKR | 93 | 1 | 93 | 93 | 31 | 11000258262374 | 18 |
| SGK196 | 42 | 99 | FL | PK | Other | Unique | 1053 | 1 | 1050 | 1050 | 350 | 17000062537825 | 46 |
| SGK396 | 43 | 100 | FL | PK | Other | Unique | 1419 | 1 | 1416 | 1416 | 472 | 5630059_1_4 | 53 |
| SGK279, PKN | 44 | 101 | FL | PK | Other | YWY3 ce | 1275 | 1 | 1272 | 1272 | 424 | 17000097259742 | 48 |
| SGK037 | 45 | 102 | FL | PK | STE | NEK | 1947 | 1 | 1947 | 1947 | 649 | 17000030265658 | 12 |
| SGK060 | 46 | 103 | FL | PK | STE | NEK | 1938 | 1 | 1935 | 1935 | 645 | 17000036897142 | 22 |
| SGK080 | 47 | 104 | FL | PK | STE | NEK | 1338 | 1 | 1338 | 1338 | 446 | 17000048119006 | 30 |
| SGK002 | 48 | 105 | FL | PK | STE | STE11 | 1829 | 327 | 1523 | 1197 | 399 | 11000257702671 | 1 |
| SGK058 | 49 | 106 | Cat | PK | STE | STE11 | 834 | 1 | 834 | 834 | 278 | 17000036877196 | 21 |
| SGK103 | 50 | 107 | partial | PK | STE | STE11 | 84 | 1 | 84 | 84 | 28 | 11000284272557 | 34 |
| SGK035 | 51 | 108 | partial | PK | STE | STE20 | 1044 | 1 | 1044 | 1044 | 348 | 17000030169905 | 11 |
| SGK075 | 52 | 109 | Cat | PK | STE | STE20 | 3318 | 1 | 3318 | 3318 | 1106 | 11000283492249 | 28 |
| SGK188, EphA9 | 53 | 110 | FL | PK | TK | RTK-11 | 3112 | 74 | 3100 | 3027 | 1009 | 17000057739161 | 45 |
| SGK040 | 54 | 111 | FL | PK | TK | Unique | 2730 | 1 | 2727 | 2727 | 909 | 11000257912897 | 14 |
| SGK390 | 55 | 112 | FL | PK-like | DAG kin | DAG kin | 3495 | 1 | 3492 | 3492 | 1164 | 17000112562523 | 52 |
| SGK007 | 56 | 113 | Cat | PK-like | GCyc | GCyc | 2652 | 1 | 2652 | 2652 | 884 | 11000257855312 | 5 |
| SGK050 | 57 | 114 | partial | PK-like | GCyc | GCyc | 144 | 1 | 144 | 144 | 48 | 17000035767558 | 19 |

| Gene Name | Ortholog | Header | Repeats | SNPs |
|---|---|---|---|---|
| SGK187, CRIK | AAC72823.1; | >CRIK- | none | 4035 = Y |
| | AAC25483.1; 181000000994928; | | | (ccgcgaggcagc |
| | AC002563.1 | | | agatcgccatgtcY); |
| | | | | 3327 = R |
| | | | | (gggttogagagct |
| | | | | gcagagR) |
| SGK064, GRK7 | AAC9500 | >GRK7- | 295 | 965 = K |
| | | | ggacccaccaaa | (catgaagcctgag |
| | | | gacagcgc 314 | aatgK); |
| | | | | 1318 = R |
| | | | | (agaaaccagagc |
| | | | | aacgcttaR) |
| SGK409, KIAA0303 | NP_032667 | >SGK409 | 2355 | 6327 = M |
| | BAA76817 | SEQ_ID_02_6671897_1_1; | agaagatgacac | (ccctccaaagact |
| | | FLv | aaatgatga | aagcaccccgaM); |
| | | predicted | 2375 | 8282 = M |
| | | from | | cacagtgaaagca |
| | | contig of | | gcagccacaagccM |
| | | SGK409 | | |
| | | (at 5′ end) | | |
| | | and | | |
| | | KIAA0303 | | |
| | | (at 3′ end)- | | |
| SGK021 | CAB76566 | >SGK021 | none | g = S |
| | BAA95027 | SGK225 | | (atgggagcS); |
| | | Incy_1110037.1; | | g7 = R |
| | | Genewise | | (agccattgggaaa |
| | | prediction; | | ggcR) |
| | | model: | | |
| | | STK_h | | |
| | | NP_060871; | | |
| | | target: | | |
| | | 173000019633053- | | |
| SGK410 | NP_002731 | >SGK410 | none | |
| | | SEQ_ID_03_6821996_50001_6; | | |
| | | FL from | | |
| | | Genewise | | |
| | | prediction | | |
| | | from HGP | | |
| | | contig | | |
| | | AL133280.12 | | |
| | | using | | |
| | | PKC, iota_h | | |
| | | NP_002731.1 | | |
| | | as a | | |
| | | model- | | |
| SGK069 | BAA36362 | >SGK069- | 1656 | |
| | | | gagagaggagg | |
| | | | aggaggaggaa | |
| | | | a 1676 | |
| SGK110 | BAA36362 | >SGK110 | none | |
| | | 1100028338966 | | |
| | | and | | |
| | | 17000077607693 | | |
| SGK053, CKLiK | Q63450 | >SGK053 | 419 | 605 = Y |
| | | 13-AUG- | tcgtccacagaga | (aaccttacagcaa |
| | | 2000 | cctcaagcc 440 | agccgY); |
| | | NM_020397.1 - | | 509 = M |
| | | Casein | | (atcagtgactttgg |
| | | kinase-like | | attgtcaaM) |
| | | kinase- | | |
| SGK124 | | >SGK124 | none | 188 = S |
| | | SEQ_ID_04_5541861_50001_6 | | (tccagatcgtgca |
| | | gi\|7263904\| | | actgctgtggS); |
| | | emb\|AL034548.25\| | | 333 = Y |
| | | HS1103G7. | | (tggccgtgctgga |
| | | [142689: | | gccctaY) |
| | | 151365].sp.tr | | |
| SGK254, CAMKKa | AAB46910 | >SGK254 | none | 1148 = R |
| | | SEQ_ID_07_4454511_1_3 | | tggtgtttcctgagg |
| | | gi\|4454511\| | | R) |
| | | gb\|AC005940.3\| | | |
| | | AC005940.[94560:118033.].sp | | |
| SGK297, CaMKIb2 | | >CaMKIb2_h | none | 77 = K |
| | | INCY_827431CB1 - | | (ggctcggctcggg |
| | | | | tgcctK) |
| SGK411, CaMKII | AAD20442 | >SGK411 | none | 15 = M; |
| delta2 | | SEQ_ID_06_2828765_1_3; | | atggcttcgaccacM |
| | | FL | | |
| | | from | | |
| | | CaMKII | | |
| | | delta2_h | | |
| | | AF071569. | | |
| | | 18-JAN- | | |
| | | 2000- | | |
| SGK027 | AAF69801 | >SGK027 | none | 45 = Y |
| | AAA97437 | CA2_GS_N_106000011351167_1, | | (tgaatggaggtgg |
| | | 17000057443791 | | cctggtgaaccccc |
| | | on | | aY) |
| | | N-term | | |
| SGK046b | AAC33487 | >SGK046b | none | |
| | AAC33487 | 11000283376057 | | |
| | AAF69801 | | | |
| SGK046c | AAC33487 | >SGk046c | none | |
| | AAC33487 | 11000284253087 | | |
| | AAF69801 | | | |
| SGK089 | AAF64455 | >SGK089 | none | |
| | AAA97437 | 13-AUG- | | |
| | | 2000 | | |
| | | AK024110 | | |
| | | (cimera?)- | | |
| SGK133 | CAA07196 | >SGK133 | none | 2003 = S |
| | | 6910558_2 | | (gcttcagggccga |
| | | INCY_984011.1 - | | gtacaaggccagt |
| | | | | gS); 1673 = S |
| | | | | (caccccccagcc |
| | | | | ccggcgS) |
| SGK004, MSK | | >SGK004- | none | |
| SGK006 | NP_056570 | >SGK006 | none | |
| | | 13-AUG- | | |
| | | 2000 = SGK006.8.gw.fa | | |
| | | (id = 10353)_PRED_CDNA_GW_HOMO_length = 789- | | |
| SGK180, SNRK | | >SNRK_h- | 1332 | 1617 = S |
| | | | ggtggaagaaga | (agccccagtgag |
| | | | tgaagaggaa | aacaatgctggtgg |
| | | | 1353 | ggS) |
| SGK386, MLCKs | | >MLCKs_h | none | 835 = M |
| | | SGK386 | | (ttttggatgattgcc |
| | | 126515.1 | | cgccaM) |
| | | gi\|8217646\| | | |
| | | emb\|AL160175.5\|AL160175. | | |
| | | [114367:128580].sp | | |
| | | HGP_7242443_3 | | |
| | | plus | | |
| | | Celera | | |
| | | gi\|8217646\| | | |
| | | emb\|AL160175.5\|AL160175. | | |
| | | [114367:128580].sp.tr | | |
| SGK003 | P70065 | >SGK003 | none | |
| | | 13-AUG- | | |
| | | 2000; FL | | |
| | | genewise | | |
| | | prediction | | |
| | | from | | |
| | | celera | | |
| | | sequence | | |
| | | 780000063066170 | | |
| | | using | | |
| | | CKIalpha_gg | | |
| | | P70065 as | | |
| | | a model; | | |
| | | manually | | |
| | | corrected | | |
| | | by | | |
| | | addition of | | |
| | | seq | | |
| | | gataactaa | | |
| | | at 1006-1014 | | |
| | | based on | | |
| | | pairwise | | |
| | | comparison | | |
| | | between | | |
| SGK066 | T42260 | >SGK066 | 486 | |
| | | | tgattttggcttggc | |
| | | | tcga 504 | |
| SGK041, NKIAMRE | | >NKIAMRE | none | 1032 = R |
| | | AF130372_1\| | | (claagtagttcagttt |
| | | AF130372 | | ttggaR); 1264 = R |
| | | Homo | | (tccacattgoggs |
| | | sapiens | | ggttctgtR); |
| | | serine- | | 1181 = Y |
| | | threonine | | (gcaaatgaaaatg |
| | | protein | | ttcatccaY) |
| | | kinase | | |
| | | (NKIAMRE) | | |
| | | mRNA, | | |
| | | complete | | |
| | | cds | | |
| SGK112 | S22745 | >SGK112 | none | |
| | | 14-AUG- | | |
| | | 2000 | | |
| SGK038, ERK7 | AAD12719 | >SGK038 | 506 | |
| | | 13-AUG- | gtgaagctgtgtg | |
| | | 2000 | actttggc 526 | |
| SGK158 | Q60938 Q92338 | >SGK158 | 1246 | 1751 = Y |
| | | Incy406057_19- | atgatgaaaactc | (atcctggttctgtgc |
| | | | tcaacaa 1265 | ctgagggagctgn |
| | | | | Y) |
| SGK429 | CAB78039 | SGK429 | 1630 | 196 = R |
| | | HGP, | acogagatggcc | (cccctgacgttctg |
| | | Icy052560.1- | atgctggtga | R); 919 = Y |
| | | | 1651 | (aggocaccaacc |
| | | | | catcY) |
| SGK152, SUDD | | >SGK152- | none | 972 = M |
| | | | | (AAACTAAAT |
| | | | | CCACGTAAG |
| | | | | ATCM) |
| SGK077 | NP_034483 | >SGK077 | none | 877 = K |
| | | [157803:160196].sp | | (ctcttcattgtgctg |
| | | | | gaatttK) |
| SGK093, Wnk3 | AAF74258 | >SGK093 | none | 2496 = K |
| | | 14-AUG- | | (gcccccaacccct |
| | | 2000; FLv | | gagccaK); |
| | | from | | 3295 = M |
| | | Genescan | | (caaaataatcagc |
| | | prediction | | aaca$$M) |
| | | from | | |
| | | Celera | | |
| | | 96000001680843; | | |
| | | corrected | | |
| | | manually | | |
| | | by | | |
| | | deletion of | | |
| | | aa | | |
| | | sequence | | |
| | | “HRLGEC | | |
| | | WQKMRR | | |
| | | RQQGAA | | |
| | | GGNFPV | | |
| | | GGSFPE | | |
| | | DVSPHQ | | |
| | | DSGYAPS | | |
| | | PR” which | | |
| | | did not fit | | |
| | | the HMM | | |
| | | prediction | | |
| SGK074 | NP_034562 | >SGK074 | none | |
| | | 13-AUG- | | |
| | | 2000 | | |
| SGK087 | AAD47290 | >SGK087 | 1264 | |
| | NP_003574 | 14-AUG- | caggatcccaag | |
| | | 2000 | gcagagga | |
| | | | 1283 | |
| SGK295, KIS | NP_058989 | >SGK295- | none | 355 = W |
| | | | | (ctggatgtcagtg$$ |
| | | | | w) |
| SGK419 | CAB70863 | >SGK419 | none | 2447 = K |
| | NP_055726 | SEQ_ID_25_5932598_100001_1; | | (attcagaa$$atctct |
| | | FL | | aaaacatggaaaa |
| | | from | | ttK) |
| | | DKFZp434P0116_h | | |
| | | NM_017593, | | |
| | | 04- | | |
| | | JUL-2000- | | |
| SGK125, MYO3A | | >MYO3A, | none | 3145 = Y |
| | | NM_017433.1\| | | (attatcacgtggag |
| | | Homo sapiens | | cagttaaa$$Y); |
| | | myosinIIIA | | 3204 = Y |
| | | mRNA- | | (gattcaagcttgtgt |
| | | | | cagagcattY) |
| SGK445 | NP_055079/59000028993040 | >SGK445- | 183 | |
| | (11.5 kb) | | taaatattttaaag | |
| | | | acaa 200 | |
| SGK127 | >NP_038599.1\|kinase_suppressor_of_ras_[Mus musculus]- | >SGK127 | none | |
| | | 17000062804843, | | |
| | | Incy_1041923, | | |
| | | Incyte_1103660, | | |
| | | 17000140021687, | | |
| | | 7276286 | | |
| | | Incyte | | |
| | | 7474604CB1- | | |
| SGK009, ANKRD3 | | >SGK009 | 427 | |
| | | | gacctcaagccc | |
| | | | gcgaacatcctgc | |
| | | | 1452 | |
| SGK421, STK22A, | NP_033461 | >SGK421 | none | |
| TSK1 | | 40000018 | | |
| | | 03622- | | |
| SGK047 | NP_004320 | >SGK047 | none | |
| SGK196 | T01289 | >SGK196 | none | 99 = R |
| | | | | (ctgctgatcatggc |
| | | | | cctgatgaatactc$$ |
| | | | | R) |
| SGK396 | CAB90410 | >SGK396 | none | |
| | CAA11152 | SEQ_ID_31_5630059_1_4 | | |
| SGK279, PKN | BAA36362 S71887 | >PKN- | none | |
| SGK037 | P51956, P51954 | >SGK037, | none | |
| | | SGK113, | | |
| | | SGK039 | | |
| | | 13-AUG- | | |
| | | 2000; FL | | |
| | | virtual | | |
| | | from | | |
| | | genewise/ | | |
| | | genescan | | |
| | | of celera | | |
| | | assembly | | |
| | | 173000036274838MDK | | |
| | | start | | |
| | | from HGP | | |
| | | 6996171 | | |
| | | (positions | | |
| | | 144536-114544) - | | |
| | | still | | |
| | | missing | | |
| | | HPN motif | | |
| | | Cterm | | |
| | | predicted | | |
| | | from | | |
| | | genescan | | |
| | | of celera | | |
| SGK060 | NP_035979 | >SGK060 | 465 | 1463 = W |
| | | 7477585CB1; | tcatcgagactta | (tgatgcatttgattc |
| | | 13- | aagtcaaagaa | ctattgtgW) |
| | | AUG-2000- | 488: 1468 | |
| | | | agtgatgaggag | |
| | | | gaagaaga | |
| | | | 1487 | |
| SGK080 | NP_002488 | >SGK080 | none | |
| | | 13-AUG- | | |
| | | 2000 | | |
| SGK002 | P36507 | >SGK002 | none | 1165 = R |
| | | SEQ_ID_35_6671973_1_4; | | (tggaggccatcttt |
| | | FL | | ggccR); |
| | | from | | 1753 = R |
| | | Genewise | | (tggtccctgccgg |
| | | prediction | | cgtgcctctR); |
| | | from HGP | | 983 = M |
| | | AC018639.8 | | (GGGGTGAG |
| | | using | | CGGCCAGCT |
| | | MPK2_h | | CATM) |
| | | P36507 as | | |
| | | a model; | | |
| | | note that | | |
| | | this gene | | |
| | | is | | |
| | | intronless | | |
| | | and | | |
| | | encodes a | | |
| | | stop | | |
| | | (position | | |
| | | x) found in | | |
| | | HGP and | | |
| | | celera | | |
| | | genomic | | |
| | | seq as | | |
| | | well as | | |
| SGK058 | A48084 AAC97114 | >SGK058 | 733 | |
| | | 13-AUG- | gaaaatgcag | |
| | | 2000- | cagactttg | |
| | | | 751 | |
| SGK103 | CAA39285 | >SGK103 | none | |
| | AAA28552 | 14-AUG- | | |
| | | 2000 | | |
| SGK035 | Q13177 | >SGK035 | 133 | 273 = Y |
| | | 481144.6 | cagaaaaagaa | (cagttttcactgcta |
| | | genewise | gactaaga 151; | cY) |
| | | on C-term- | 695 | |
| | | | actggatggcacc | |
| | | | agaagt 713 | |
| SGK075 | NP_059129 | >SGK075 | none | 877 = R |
| | P50527 | | | (tttctgcaaaaaca |
| | | | | gctggccR) |
| SGK188, EphA9 | | >SGK188 | none | 2319 = R |
| | | Incyte_7474721C81 | | (gtggctgggcaac |
| | | | | tgatggR) |
| SGK040 | P41243 T33475 | >SGK040 | 460 | 1889 = R |
| | | SEQ_ID_40_6227040_1_1 | tttctttcaaaaaa | (ggttgtgccccatg |
| | | | aaggaa 479 | gcctatggttgaR) |
| | | | | & 1004 = Y |
| | | | | (gacttgattttggct |
| | | | | gaccY) |
| SGK390 | Q64398 | >SGK390 | none | 1314 = R |
| | | 14-AUG- | | (gggaggttcatat |
| | | 2000 | | acgatgacaccca |
| | | | | R) |
| SGK007 | T42260 | >SGK007 | none | |
| SGK050 | P16067 | >SGK050 | none | |
| | | 13-AUG- | | |
| | | 2000 | | |

Table 2 lists the following features of the genes described in this application: chromosomal localization, single nucleotide polymorphisms (SNPs), representation in dbEST, and repeat regions. From left to right the data presented is as follows: “Gene Name”, “ID#na”, “ID#aa”, “FL/Cat”, “Superfamily”, “Group”, “Family”, “Chromosome”, “SNPs”, “dbEST_hits”, & “Repeats”. The contents of the first 7 columns (i.e.,. “Gene Name”, “ID#na”, “ID#aa”, “FL/Cat”, “Superfamily”, “Group”, “Family”) are as described above for Table 1. “Chromosome” refers to the cytogenetic localization of the gene. Information in the “SNPs” column describes the nucleic acid position and degenerate nature of candidate single nucleotide polymorphisms (SNPs). For example, for SGK386, the “SNPs” column contains “835=M”, indicating that there are instances of both a C and an A (M=C or A) at position 835. “dbESThits” lists accession numbers of entries in the public database of ESTs (dbEST, http://www.ncbi.nlm.nih.gov/dbEST/index.html) that contain at least 100 bp of 100% identity to the corresponding gene. These ESTs were identified by blastn of dbEST. “Repeats” contains information about the location of short sequences, approximately 20 bp in length, that are of low complexity and that are present in several distinct genes. These repeats were identified by blastn of the DNA sequence against the non-redundant nucleic acid database at NCBI (nrna). To be included in this repeat column, the sequence typically could have 100% identity over its length and typically is present in at least 5 different genes. (SNPs are disclosed as SEQ ID NOS 203-268, respectively in order of appearance).

TABLE 2
CHR, SNPs, dbEST, Repeats
413406_1.xls

| Gene Name | ID#na | ID#aa | FL/Cat | Superfamily | Group | Family | Chromosome | SNPs | dbEST_hits | Repeats | Genomic_ID | sort1 |
|---|---|---|---|---|---|---|---|---|---|---|---|---|
| SGK187, CRIK | 1 | 58 | FL | PK | AGC | DMPK | 12q24.23 | 2874 = R | BE909486, | none | 4926912_1_1 | 44 |
| | | | | | | | | (ss1337340); | BE875297, | | | |
| | | | | | | | | 2883 = Y | AW605350 | | | |
| | | | | | | | | (rs904655); | | | | |
| | | | | | | | | 3327 = R | | | | |
| | | | | | | | | (ss1581624) | | | | |
| SGK064, GRK7 | 2 | 59 | FL | PK | AGC | GRK | 3q24 | 965 = K; 1318 = R | none | 295-314 | 11000284009826 | 23 |
| SGK409, | 3 | 60 | FL | PK | AGC | MAST | 5q12.1 | 6282 = M; | BE515326, | 2355-2375 | 6671897_1_1 | 54 |
| KIAA0303 | | | | | | | | 6327 = M | BE267294, | | | |
| | | | | | | | | | AA926642 | | | |
| SGK021 | 4 | 61 | Cat | PK | AGC | Mo3C11.1 | 5q31.2 | 9 = S; 97 = R | none | none | 17000028181153 | 8 |
| | | | | | | ce | | | | | | |
| SGK410 | 5 | 62 | FL | PK | AGC | PKC | Xq23 | | none | none | 7018620_2 | 55 |
| SGK069 | 6 | 63 | Cat | PK | AGC | Unique | 19p11-13 | 1180 = S | none | 1656-1678 | 11000283338966 | 26 |
| | | | | | | | | (ss1317629); | | | | |
| | | | | | | | | 210 = Y | | | | |
| | | | | | | | | (ss1688813) | | | | |
| SGK110 | 7 | 64 | partial | PK | AGC | Unique | 19q13.4 | 597 = R | none | none | 11000284702500 | 35 |
| | | | | | | | | (rs654439); | | | | |
| | | | | | | | | 252 = Y | | | | |
| | | | | | | | | (ss681406) | | | | |
| SGK053, CKLIK | 8 | 65 | FL | PK | CAMK | AMPK | 10p14 | 605 = Y; 509 = M | BE266955, | 419-440 | 17000035790284 | 20 |
| | | | | | | | | | AI923704, | | | |
| | | | | | | | | | AW501047 | | | |
| SGK124 | 9 | 66 | FL | PK | CAMK | AMPK | 20p12.2-p13 | 188 = S; 333 = Y | BF026145, | none | 17000047891899 | 37 |
| | | | | | | | | | BF298893 | | | |
| SGK254, | 10 | 67 | FL | PK | CAMK | CAMK | 17p13.3 | 555 = R | BE783149 | none | 4454511_1_3 | 47 |
| CAMKKa | | | | | | | | (ss84265); | | | | |
| | | | | | | | | 1148 = R | | | | |
| SGK297, | 11 | 68 | FL | PK | CAMK | CAMK | Xq28 | 77 = K | AI696123, | none | 17000113122540 | 50 |
| CaMKIb2 | | | | | | | | | AI141657 | | | |
| SGK411, | 12 | 69 | FL | PK | CAMK | CAMK | 4q25 | 15 = M; 1367 = S | AW502248, | none | 2828765_1_3 | 56 |
| CaMKII delta2 | | | | | | | | (ss1531091) | AW504981, | | | |
| | | | | | | | | | AA316038 | | | |
| SGK027 | 13 | 70 | FL | PK | CAMK | EMK | 5q11-q11.1 | 45 = Y | BE551926, | none | 17000029868654 | 9 |
| | | | | | | | | | AI609751, | | | |
| | | | | | | | | | AW138653 | | | |
| SGK046b | 14 | 71 | partial | PK | CAMK | EMK | 3p24.1 | | none | none | 11000284253087 | 16 |
| SGK046c | 15 | 72 | partial | PK | CAMK | EMK | 3p24.1 | | none | none | 11000284253087 | 17 |
| SGK089 | 16 | 73 | partial | PK | CAMK | EMK | 3p25.3 | | none | none | 11000283986586 | 32 |
| SGK133, | 17 | 74 | FL | PK | CAMK | EMK | 7p11.2-p21 | 2003 = S; 1673 = S | BE222941 | none | 6671894_1_1 | 40 |
| N83965 | | | | | | | | | | | | |
| SGK004, MSK | 18 | 75 | FL | PK | CAMK | EMK-1 | 21q22.3 | 1853 = Y | AW503500 | none | 11000257744647 | 3 |
| | | | | | | | | (ss571239) | | | | |
| SGK006 | 19 | 76 | Cat | PK | CAMK | EMK-1 | 16q16.1 | 4 = S (ss1609852) | none | none | 11000257829454 | 4 |
| SGK180, SNRK | 20 | 77 | FL | PK | CAMK | EMK-1 | 3p21.31 | 1817 = S | AA447812, | 1332-1353 | 17000057577785 | 43 |
| | | | | | | | | | AI379954, | | | |
| | | | | | | | | | AA199639 | | | |
| SGK386, | 21 | 78 | FL | PK | CAMK | MLCK | 20q11.1 | 835 = M | AA197072, | none | 17000140438265 | 51 |
| MLCKs | | | | | | | | | R02824 | | | |
| SGK003 | 22 | 79 | FL | PK | CKI | CKI | 13q14.11 | | none | none | 11000257741254 | 2 |
| S6K066 | 23 | 80 | Cat | PK | CKI | CKI | 15q15 | | AA234451 | 486-504 | 11000283296349 | 24 |
| SGK041, | 24 | 81 | FL | PK | CMGC | CDK | 5q31.1 | 1033 = R; | AI684625, | none | 11000257953829 | 15 |
| NKIAMRE | | | | | | | | 1181 = Y; 1284 = R | AI894352, | | | |
| | | | | | | | | | AI912547 | | | |
| SGK112 | 25 | 82 | FL | PK | CMGC | CDK | CHR2 | | AA626859 | none | 17000035915087 | 36 |
| SGK038, ERK7 | 26 | 83 | FL | PK | CMGC | MAPK | na | | BE464580, | 508-528 | 17000030278391 | 13 |
| | | | | | | | | | AI049667 | | | |
| SGK158 | 27 | 84 | FL | PK | Microbial | ABC1 | 1q42.11-42.2 | 1752 = Y | BE797060, | 1246-1265 | 11000283551497 | 42 |
| | | | | | PK | | | (ss1529336) | AW006971, | | | |
| | | | | | | | | | AI819411 | | | |
| SGK429 | 28 | 85 | FL | PK | Microbial | ABC1 | 7q34-35 | 195 = R; 919 = Y | BE877541, | 1630-1651 | 5001549_3 | 60 |
| | | | | | PK | | | (ss1549835); | BE745459, | | | |
| | | | | | | | | 1865 = Y | BE259124 | | | |
| | | | | | | | | (ss1517749) | | | | |
| SGK152, SUDD | 29 | 86 | FL | PK | Microbial | RI01 | 18p11.1 | 972 = M | BE621277, | none | 11000258249295 | 41 |
| | | | | | PK | | | | AA452706, | | | |
| SGK077 | 30 | 87 | FL | PK | Other | C26C2_ce | 17p13.3 | 390 = Y | AA504583, | none | 11000283672012 | 29 |
| | | | | | | | | (ss1658885); | AW752337 | | | |
| | | | | | | | | 611 = R | | | | |
| | | | | | | | | (ss1629760); | | | | |
| | | | | | | | | 985 = Y | | | | |
| | | | | | | | | (ss1629759) | | | | |
| SGK093, Wnk3 | 31 | 88 | FL | PK | Other | C26C2_ce | 17q21.1-2 | 3279 = K; | BF000369, | none | 11000284063441 | 33 |
| | | | | | | | | 4076 = M | AI650786, | | | |
| | | | | | | | | | AA872586 | | | |
| SGK074 | 32 | 89 | Cat | PK | Other | DYRK | 19p12-19q13 | | none | none | 11000283476699 | 27 |
| SGK087 | 33 | 90 | FL | PK | Other | DYRK | 12p13.3 | 269 = R (ss68136) | BE243995 | 1264-1283 | 17000036171996 | 31 |
| SGK295, KIS | 34 | 91 | FL | PK | Other | DYRK | 1q23.3 | 355 = W | BE895119, | none | 17000113079883 | 49 |
| | | | | | | | | | AI221234, | | | |
| | | | | | | | | | BE145607 | | | |
| SGK419 | 35 | 92 | FL | PK | Other | NAK | 4q24 | | AI735391, | none | 5932598_100001_1 | 57 |
| | | | | | | | | | AW967339, | | | |
| | | | | | | | | | AA703517 | | | |
| SGK125, | 36 | 93 | FL | PK | Other | NinaC | 10p12.32 | 3145 = Y; 3204 = Y | AW196373, | none | 17000078090910 | 38 |
| MYO3A | | | | | | | | | AA476897 | | | |
| SGK445 | 37 | 94 | partial | PK | Other | PLK | na | | none | 183-200 | 4000001803382 | 61 |
| SGK127 | 38 | 95 | FL | PK | Other | RAF | 12q24.21 | 501 = S | none | none | 17000062804843 | 39 |
| | | | | | | | | (ss2005786) | | | | |
| SGK009, | 39 | 96 | FL | PK | Other | RIP | 21q22.3 | | none | 427.452 | 11000258161587 | 6 |
| ANKRD3 | | | | | | | | | | | | |
| SGK421, | 40 | 97 | FL | PK | Other | STK22A | 5q31.1 | 510 = M | none | none | 6759077_1_4 | 59 |
| STK22A, TSK1 | | | | | | | | (ss2055126); | | | | |
| | | | | | | | | 279 = R | | | | |
| | | | | | | | | (ss2055125) | | | | |
| SGK047 | 41 | 98 | partial | PK | Other | STKR | 10p11.21 | | none | none | 11000258262374 | 18 |
| SGK196 | 42 | 99 | FL | PK | Other | Unique | na | 99 = R | none | none | 17000062537825 | 46 |
| SGK396 | 43 | 100 | FL | PK | Other | Unique | na | | AL048857, | none | 5630059_1_4 | 53 |
| | | | | | | | | | AL048858, T09068 | | | |
| SGK279, PKN | 44 | 101 | FL | PK | Other | YWY3 ce | 16q22.3 | 665 = Y | none | none | 17000097259742 | 48 |
| SGK037 | 45 | 102 | FL | PK | STE | NEK | 13q14.12 | | AA393108 | 886-912 | 17000030265658 | 12 |
| SGK060 | 46 | 103 | FL | PK | STE | NEK | 3q22.1 | 1483 = W | BE388672, | 465-488; | 17000036897142 | 22 |
| | | | | | | | | | AA412114 | 1468-1487 | | |
| SGK080 | 47 | 104 | FL | PK | STE | NEK | 22q11.2 | 1159 = R | none | none | 17000048119006 | 30 |
| | | | | | | | | (ss1367671); | | | | |
| | | | | | | | | 422 = R | | | | |
| | | | | | | | | (ss1855009) | | | | |
| SGK002 | 48 | 105 | FL | PK | STE | STE11 | 7q32.2 | 1165 = R; 983 = M; | none | none | 11000257702671 | 1 |
| | | | | | | | | 443 = K; 1245 = Y; | | | | |
| | | | | | | | | 1261 = K; 1753 = R | | | | |
| SGK058 | 49 | 106 | Cat | PK | STE | STE11 | 2q21.2 | | none | 733-751 | 17000036877196 | 21 |
| SGK103 | 50 | 107 | partial | PK | STE | STE11 | 5p14.3 | | none | none | 11000284272557 | 34 |
| SGK035 | 51 | 108 | partial | PK | STE | STE20 | CHR15 | 2273 = Y | none | 133-151; 695-713 | 17000030169905 | 11 |
| SGK075 | 52 | 109 | Cat | PK | STE | STE20 | 2q31.1 | 889 = R | none | none | 11000283492249 | 28 |
| SGK188, | 53 | 110 | FL | PK | TK | RTK-11 | 1p34.1-34.3 | 2104 = Y | none | none | 17000057739161 | 45 |
| EphA9 | | | | | | | | (ss1986120); | | | | |
| | | | | | | | | 2319 = R | | | | |
| SGK040 | 54 | 111 | FL | PK | TK | Unique | 12q12 | 1869 = R, 1004 = Y | BE177830, | 460-479 | 11000257912897 | 14 |
| | | | | | | | | | AF114068, | | | |
| | | | | | | | | | AA263608 | | | |
| SGK390 | 55 | 112 | FL | PK-like | DAG kin | DAG kin | 13q14.2 | 1314 = R | BE715967, | none | 17000112562523 | 52 |
| | | | | | | | | | AI333565, | | | |
| | | | | | | | | | AW052032 | | | |
| SGK007 | 56 | 113 | Cat | PK-like | GCyc | GCyc | 10q26.11 | | none | none | 11000257855312 | 5 |
| SGK050 | 57 | 114 | partial | PK-like | GCyc | GCyc | 9p13.1-2 | | none | none | 17000035767558 | 19 |

| Gene Name | Ortholog | Header | Repeats | SNPs |
|---|---|---|---|---|
| SGK187, CRIK | AAC72823.1; | >CRIK- | none | 3327 = R |
| | AAC25483.1; | | | (gggttcgagagctgcagagR) |
| | 181000000994928; | | | dbSNP ss1581624; |
| | AC002563.1 | | | 2883 = Y |
| | | | | (agagaccacagcagaagcY) |
| | | | | dbSNP rs904655; |
| | | | | 2874 = R |
| | | | | (gctggaagagaccacR) |
| | | | | dbSNP ss1337340) |
| SGK064, GRK7 | AAC9500 | >GRK7- | 295 | 965 = K |
| | | | ggacccaccaaagacagcgc | (catgaagcctgagaatgK); |
| | | | 314 | 1318 = R |
| | | | | (agaaaccagagcaacgctta |
| | | | | R) |
| SGK409, | NP_032667 | >SGK409 | 2355 | 6327 = M |
| KIAA0303 | BAA76817 | SEQ_ID_02_6671897_1_1; | agaagatgacacaaatgatga | (ccctccaaagactaagcaccc |
| | | FLv | 2375 | cgaM); 6282 = M |
| | | predicted | | cacagtgaaagcagcagccac |
| | | from contig | | aagccM |
| | | of SGK409 | | |
| | | (at 5′ end) | | |
| | | and | | |
| | | KIAA0303 | | |
| | | at 3′ end)- | | |
| SGK021 | CAB76566 | >SGK021 | none | 9 = S (atgggagcS); 97 = R |
| | BAA95027 | SGK225 | | (agccattgggaaaggcR) |
| | | Incy_1110037.1; | | |
| | | Genewise | | |
| | | prediction; | | |
| | | model: | | |
| | | STK_h | | |
| | | NP_060871; | | |
| | | target: | | |
| | | 173000019633053- | | |
| SGK410 | NP_002731 | >SGK410 | none | |
| | | SEQ_ID_03_6821996_50001_6; | | |
| | | FL | | |
| | | from | | |
| | | Genewise | | |
| | | prediction | | |
| | | from HGP | | |
| | | contig | | |
| | | AL133280.12 | | |
| | | using | | |
| | | PKC, iota_h | | |
| | | NP_002731.1 | | |
| | | as a model- | | |
| SGK069 | BAA36362 | >SGK069- | 1656 | 1180 = S |
| | | | gagagaggaggaggaggagg | (ctggttcggcctggccS) |
| | | | aaa 1678 | dbSNP ss1317629; |
| | | | | 210 = Y |
| | | | | (tgcaggacggggaY) |
| | | | | dbSNP ss1688813 |
| SGK110 | BAA36362 | >SGK110 | none | 597 = R |
| | | 11000283338966 | | (aacgtgctggtcttcgacccR) |
| | | and | | dbSNP rs654439; 252 = Y |
| | | 17000077607693 | | (ccgcgtgctcctY) dbSNP |
| | | | | ss661406 |
| SGK053, CKLIK | Q63450 | >SGK053 | 419 | 605 = Y |
| | | 13-AUG- | tcgtccacagagacctcaagcc | (aaccttacagcaaagccgY); |
| | | 2000 | 440 | 509 = M |
| | | NM_020397.1 - | | (atcagtgactttggattgtcaaM) |
| | | Casein | | |
| | | kinase-like | | |
| | | kinase - | | |
| SGK124 | | >SGK124 | none | 188 = S |
| | | SEQ_ID_04_5541861_50001_6 | | (tccagatcgtgcaactgctgtgg |
| | | gi\|7263904\| | | S); 333 = Y |
| | | emb\|AL034548.25\| | | (tggccgtgctggagccctaY) |
| | | HS1103G7. | | |
| | | [142689:151365].sp.tr | | |
| SGK254, | AAB46910 | >SGK254 | none | 555 = R (gtcgccctcccccR) |
| CAMKKa | | SEQ_ID_07_4454511_1_3 | | dbSNP ss84265; |
| | | gi\|4454511\| | | 1148 = R |
| | | gb\|AC005940.3\| | | (tggtgtttcctgaggR) |
| | | AC005940. | | |
| | | [94560:118033].sp | | |
| SGK297, | | >CaMKIb2h | none | 77 = K |
| CaMKIb2 | | INCY_827431CB1- | | (ggctcggctcgggtgcctK) |
| SGK411, | AAD20442 | >SGK411 | none | 15 = M; atggcttcgaccacM; |
| CaMKII delta2 | | SEQ_ID_06_2828765_1_3; | | 1387 = S |
| | | FL from | | (ccgggatggaaagtggS) |
| | | CaMKII | | dbSNP ss1531091 |
| | | delta2_h | | |
| | | AF071569, | | |
| | | 18-JAN- | | |
| | | 2000- | | |
| SGK027 | AAF69801 | >SGK027 | none | 45 = Y |
| | AAA97437 | CA2_GS_N_106000011351167_1. | | (tgaatggaggtggcctggtgaa |
| | | 17000057443791 | | cccccaY) |
| | | on N- | | |
| | | term | | |
| SGK046b | AAC33487 | >SGK046b | none | |
| | AAC33487 | 11000283376057 | | |
| | AAF69801 | | | |
| SGK046c | AAC33467 | >SGk046c | none | |
| | AAC33487 | 11000284253087 | | |
| | AAF69801 | | | |
| SGK089 | AAF64455 | >SGK089 | none | |
| | AAA97437 | 13-AUG- | | |
| | | 2000 | | |
| | | AK024110 | | |
| | | (cimera?)- | | |
| SGK133, | CAA07196 | >SGK133 | none | 2003 = S |
| N83965 | | 6910558_2 | | (gcttcagggccgagtacaagg |
| | | INCY_984011.1 - | | ccagtgS); 1673 = S |
| | | | | (caccccccagccccggcgS) |
| SGK004, MSK | | >SGK004- | none | 1853 = Y |
| | | | | (aggtgtgccaggccccY) |
| | | | | dbSNP ss571239; |
| SGK006 | NP_056570 | >SGK006 | none | Sctctgtatttcaggaagctct |
| | | 13-AUG- | | 4 = S, dbSNP ss1009652 |
| | | 2000 = SGK006.8.gw.fa | | |
| | | (id = 10353)_PRED_CDNA_GW_HOMO_length = 789- | | |
| SGK180, SNRK | | >SNRK h- | 1332 | 1817 = S |
| | | | ggtggaagaagatgaagagga | (agccccagtgagaacaatgct |
| | | | a 1353 | ggtggggS) |
| SGK386, | | >MLCKs_h | none | 835 = M |
| MLCKs | | SGK386 | | (ttttggatgattgcccgocaM) |
| | | 126515.1 | | |
| | | gi\|8217646\| | | |
| | | emb\|AL160175.5\| | | |
| | | AL160175. | | |
| | | [114367:128580].sp | | |
| | HGP_7242443_3 | | | |
| | | plus | | |
| | | Celera | | |
| | | gi\|8217646\| | | |
| | | emb\|AL160175.5\| | | |
| | | AL160175.5. | | |
| | | [114367:128580].sp.tr | | |
| SGK003 | P70065 | >SGK003 | none | |
| | | 13-AUG- | | |
| | | 2000; FL | | |
| | | genewise | | |
| | | prediction | | |
| | | from celera | | |
| | | sequence | | |
| | | 780000063066170 | | |
| | | using | | |
| | | CKIalpha_g | | |
| | | g P70065 as | | |
| | | a model: | | |
| | | manually | | |
| | | corrected by | | |
| | | addition of | | |
| | | seq | | |
| | | gataactaa at | | |
| | | 1006-1014 | | |
| | | based on | | |
| | | pairwise | | |
| | | comparison | | |
| | | between as | | |
| | | seq for | | |
| | | SGK003 and | | |
| | | CKI, alpha_h | | |
| | | NP_001883.2 | | |
| | | and CKI, | | |
| S6K066 | T42260 | >SGK068 | 486 tgattttggcttggctcga | |
| | | | 504 | |
| SGK041, | | >NKIAMRE | none | 1033 = R |
| NKIAMRE | | AF130372.1\| | | (ctaagtagttcagttttgggaR); |
| | | AF130372 | | 1284 = R |
| | | Homo | | (tccacattgcggaggttctgtR); |
| | | sapiens | | 1181 = Y |
| | | serine- | | (gcaaatgaaaatgttcatccta |
| | | threonine | | Y) |
| | | protein | | |
| | | kinase | | |
| | | (NKIAMRE) | | |
| | | mRNA, | | |
| | | complete | | |
| | | cds | | |
| SGK112 | S22745 | >SGK112 | none | |
| | | 14-AUG- | | |
| | | 2000 | | |
| SGK038, ERK7 | AAD12719 | >SGK038 | 508 | |
| | | 13-AUG- | gtgaagctgtgtgactttggc | |
| | | 2000 | 528 | |
| SGK158 | Q60936 | >SGK158 | 1246 | 1752 = Y |
| | Q92338 | Incy406057_19- | atgatgaaaactctcaacaa | (atcctggttctgtgcctgaggga |
| | | | 1265 | gctgttY) dbSNP |
| | | | | ss1529336 |
| SGK429 | CAB76039 | >SGK429 | 1630 | 196 = R |
| | | HGP, | accgagatggccatgctggtga | (cccctgacgttctgR); 919 = (aggccaccaacctcatcY) |
| | | Incy052560.1- | 1651 | dbSNP ss1549835; |
| | | | | 1865 = Y |
| | | | | (CCTCCTCACGGGCCY) |
| | | | | dbSNP ss1517749; |
| SGK152, SUDD | | >SGK152- | none | 972 = M |
| | | | | (AAACTAAATCCACGTA |
| | | | | AGATCM) |
| SGK077 | NP_034483 | >SGK077 | none | 390 = Y (agcacaccctgY) |
| | | [157803:160196].sp | | dbSNP ss1658885; |
| | | | | 611 = R |
| | | | | (cagccgtcccgagcgR) |
| | | | | dbSNP ss1629760; |
| | | | | 985 = Y |
| | | | | (ttgtgccaaggggaaY) |
| | | | | dbSNP ss1629759 |
| SGK093, Wnk3 | AAF74258 | >SGK093 | none | 3279 = K |
| | | 14-AUG- | | (gcccccaacccctgagccaK): |
| | | 2000; FLv | | 4076 = M |
| | | from | | (caaaataatcagcaacattM) |
| | | Genescan | | |
| | | prediction | | |
| | | from Celera | | |
| | | 96000001680843; | | |
| | | corrected | | |
| | | manually by | | |
| | | deletion of | | |
| | | aa sequence | | |
| | | *HRLGECW | | |
| | | QKMRRRQ | | |
| | | QGAAGGN | | |
| | | FPVGGSFP | | |
| | | EDVSPHQD | | |
| | | SGYAPSPR | | |
| | | * which did | | |
| | | not fit the | | |
| | | HMM | | |
| | | predicltion | | |
| | | and did not | | |
| | | align to | | |
| | | NEK7. | | |
| | | Genescan | | |
| SGK074 | NP_034562 | >SGK074 | none | |
| | | 13-AUG- | | |
| | | 2000 | | |
| SGK087 | AAD47290 | >SGK087 | 1264 | 269 = R |
| | NP_003574 | 14-AUG- | caggatcccaaggcagagga | (acttcacgagcagtcaggcR) |
| | 2000 | 1283 | dbSNP ss68136 | |
| SGK295, KIS | NP_058989 | >SGK295- | none | 356 = W |
| | | | | (ctggatgtcagtgttW) |
| SGK419 | CAB70863 | >SGK419 | none | |
| | NP_055726 | SEQ_ID_25_5932598_1_100001_1; | | |
| | | FL | | |
| | | from | | |
| | | DKFZp434P | | |
| | | 0116_h | | |
| | | NM_017593, | | |
| | | 04-JUL- | | |
| | | 2000- | | |
| SGK125, | | >MYO3A, | none | 3145 = Y |
| MYO3A | | NM_017433.1\| | | (attatcacgtggagcagttaaat |
| | | Homo sapiensmyosin$$ | | Y): 3204 = Y |
| | | AmRNA- | | (gattcaagcttgtgtcagagcat |
| | | | | Y) |
| SGK445 | NP_055079/ | >SGK445- | 183 taaatattttaaagacaa | |
| | 59000028993040 | | 200 | |
| | (11.5 kb) | | | |
| SGK127 | >NP_038599.1\| | >SGK127 | none | 501 = S (tccagtggcccacS) |
| | kinase_suppressor_of_ras_[Mus_musculus]- | 17000062804843, | | dbSNP ss2005786; |
| | | Incy_1041923, | | |
| | | Incyte_1103660, | | |
| | | 17000140021687, | | |
| | | 7276286 | | |
| | | Incyte | | |
| | | 7474604CB1- | | |
| SGK009, | | >SGK009- | 427 | |
| ANKRD3 | | | gacctcaagcccgcgaacatcc | |
| | | | tgct 452 | |
| SGK421, | NP_033461 | >SGK421 | none | 510 = M |
| STK22A, TSK1 | | 4000001803622- | | (ACAGTGGTCGAATGG |
| | | | | CM) dbSNP ss2055126; |
| | | | | 279 = R |
| | | | | (gtcatggagctcgcR) |
| | | | | dbSNP ss2055125 |
| SGK047 | NP_004320 | >SGK047 | none | |
| SGK196 | T01289 | >SGK196 | none | 99 = R |
| | | | | (ctgctgatcatggccctgatgaa |
| | | | | tactctR) |
| SGK396 | CAB90410 | >SGK396 | none | |
| | CAA11152 | SEQ_ID_31_5630059_1_4 | | |
| SGK279, PKN | BAA36362 | >PKN- | none | 665 = Y |
| | S71887 | | | (CCTGAGGTGTGCCAG |
| | | | | GY) |
| SGK037 | P51956, | >SGK037, | 886 | |
| | P51954 | SGK113, | aggtgtgagccactgcacccag | |
| | | SGK039 13 | cctgt 912 | |
| | | AUG-2000; | | |
| | | FL virtual | | |
| | | from | | |
| | | genewise/genescan | | |
| | | of | | |
| | | celera | | |
| | | assembly | | |
| | | 1730000362 | | |
| | | 74838MDK | | |
| | | start from | | |
| | | HGP | | |
| | | 6996171 | | |
| | | (positions | | |
| | | 144536-114544)- | | |
| | | still missing | | |
| | | HPN motif | | |
| | | Cterm | | |
| | | predicted | | |
| | | from | | |
| | | genescan of | | |
| | | celera | | |
| | | assembly | | |
| | | 1730000362 | | |
| SGK060 | NP_035979 | >SGK060 | 465 | 1463 = W |
| | | 7477585CB1; | tcatcgagacttaaagtcaaaga | (tgatgcatttgattcctattgtgW |
| | | 13-AUG- | a 488; 1468 | |
| | | 2000- | agtgatgaggaggaagaaga | |
| | | | 1467 | |
| SGK080 | NP_002488 | >SGK080 | none | 1159 = R |
| | | 13-AUG- | | (aagttcatttcagtggR) |
| | | 2000 | | dbSNP as 1367671; |
| | | | | 422 = R |
| | | | | (tactgtagtgcgtcgggR) |
| | | | | dbSNP as 1855009 |
| SGK002 | P36507 | >SGK002 | none | 1165 = R |
| | | SEQ_ID_35_6671973_1_4; | | (tggaggccatctttggccR); |
| | | FL from | | 1753 = R |
| | | Genewise | | (tggtccctgccggcgtgcctctR); |
| | | prediction | | 983 = M |
| | | from HGP | | (GGGGTGAGCGGCCA |
| | | AC018639.8 | | GCTCATM); 443 = K, |
| | | using | | (ACCTGGTGGACCTGC |
| | | MPK2_h | | AGAAK); 1245 = Y |
| | | P36507 as a | | (GCGCCCCAACAGCG |
| | | model; note | | GTY); 1261 = K |
| | | that this | | (GGGATGGACAgccK) |
| | | gene is | | |
| | | intronless | | |
| | | and encodes | | |
| | | a stop | | |
| | | (position x) | | |
| | | found in | | |
| | | HGP and | | |
| | | celera | | |
| | | genomic seq | | |
| | | as well as | | |
| | | Monsanto | | |
| | | ESts; | | |
| | | therefore, it | | |
| | | may be a | | |
| SGK058 | A48084 | >SGK058 | 733 | |
| | AAC97114 | 13-AUG- | gaaaatgcagcagacttt | |
| | | 2000- | g 751 | |
| SGK103 | CAA39285 | >SGK103 | none | |
| | AAA28552 | 14-AUG- | | |
| | | 2000 | | |
| SGK035 | Q13177 | >SGK035 | 133 | 273 = Y |
| | | 481144.6 | cagaaaaagaagactaaga | (cagttttcactgctacY) |
| | | genewise on | 151; 695 | |
| | | C-term - | actggatggcaccagaagt | |
| | | | 713 | |
| SGK075 | NP_059129 | >SGK075 | none | 889 = R |
| | P50527 | | | (tttctgcaaaaacagctggccR) |
| SGK188, | | >SGK188 | none | 2319 = R |
| EphA9 | | Incyte_7474721CB1- | | (gtggctgggcaactgatggR); |
| | | | | 2104 = Y (gtagccgtgcaY) |
| | | | | dbSNP as 1986120; |
| SGK040 | P41243 | >SGK040 | 460 | 1869 = R |
| | T33475 | SEQ_ID_40_6227040_1_1 | tttctttcaaaaaaaaggaa | (ggttgtgccccatggcctatggtt |
| | | | 479 | gaR); 1004 = Y |
| | | | | (gacttgattttggctgaccY) |
| SGK390 | Q64398 | >SGK390 | none | 1314 = R |
| | | 14-AUG- | | (gggaggttcatatgacgatgac |
| | | 2000 | | acccaR) |
| SGK007 | T42260 | >SGK007 | none | |
| SGK050 | P16067 | >SGK050 | none | |
| | | 13-AUG- | | |
| | | 2000 | | |

Table 3 lists the extent and the boundaries of the kinase catalytic domains. The column headings are: “Gene Name”, “ID#na”, “ID#aa”, “FL/Cat”, “Profile_start”, “Profile_end”, “Kinase_start”, “Kinase_end”, and “profile”. The contents of the first 7 columns (i.e.,. “Gene Name”, “ID#na”, “ID#aa”, “FL/Cat”, “Superfamily”, “Group”, “Family”) are as described above for Table 1. “Profile Start”, “Profile End”, “Kinase Start” and “Kinase End” refer to data obtained using a Hidden-Markov Model to define catalytic range boundaries. The profile has a length of 261 amino acids, corresponding to the complete protein kinase catalytic domain. Proteins in which the profile recognizes a full length catalytic domain have a “Profile Start” of 1 and a “Profile End” of 261. Genes which have a partial catalytic domain will have a “Profile Start” of greater than 1 (indicating that the beginning of the kinase domain is missing, and/or a “Profile End” of less than 261 (indicating that the C-terminal end of the kinase domain is missing). The boundaries of the catalytic domain within the overall protein are noted in the “Kinase Start” and “Kinase End” columns. “Profile” indicates whether the complete or “Smith Waterman” (partial). Starting from a multiple sequence alignment of kinase catalytic domains, two hidden Markov models were built. One of them allows for partial matches to the catalytic domain; this is a “local” HMM, similar to Smith-Waterman alignments in sequence matching. The other “complete” model allows matches only to the complete catalytic domain; this is a “global” HMM similar to Needleman-Wunsch alignments in sequence matching. The Smith Waterman local model is more specific, allowing for fragmentary matches to the kinase catalytic domain whereas the global “complete” model is more sensitive, allowing for remote homologue identification. (GAATCTTGGATCCAGTAA is disclosed as SEQ ID NO: 277).

TABLE 3
Kinase Domains
413406_1.xls

| Gene Name | ID#na | ID#aa | FL/Cat | Profile_start | Profile_end | Kinase_start | Kinase_end | Profile | Comparison | Genomic_ID | Cel Sort |
|---|---|---|---|---|---|---|---|---|---|---|---|
| SGK187, CRIK | 1 | 58 | FL | 1 | 261 | 98 | 361 | Complete | | 4926912_1_1 | 44 |
| SGK064, GRK7 | 2 | 59 | FL | 1 | 261 | 191 | 454 | Complete | | 11000284009826 | 23 |
| SGK409, KIAA0303 | 3 | 60 | FL | 1 | 261 | 470 | 743 | Complete | | 6671897_1_1 | 54 |
| SGK021 | 4 | 61 | Cat | 1 | 261 | 23 | 263 | Complete | MG N S | 17000028181153 | 8 |
| | | | | | | | | | KPPVFDENE+V | | |
| | | | | | | | | | NFDHF+ILRAIG | | |
| | | | | | | | | | KG FGKVCIVQK | | |
| | | | | | | | | | DTKKMYAMKY | | |
| | | | | | | | | | MNKQKCVER+E | | |
| | | | | | | | | | VRNVF+ELQIM | | |
| | | | | | | | | | QGLEHPFLVNL | | |
| | | | | | | | | | WYSFQDEEDM | | |
| | | | | | | | | | FMVVDLLLGGD | | |
| | | | | | | | | | LRYHLQQNVHF | | |
| | | | | | | | | | E TVKL+ICEL | | |
| | | | | | | | | | +AL+YLQ | | |
| | | | | | | | | | IIHRD+KPDNILL | | |
| | | | | | | | | | DEHGHVHITDF | | |
| | | | | | | | | | NIA +L + | | |
| | | | | | | | | | ++MAGTKPYMA | | |
| | | | | | | | | | PE+F G | | |
| | | | | | | | | | GYS+ | | |
| | | | | | | | | | VDWWSLGVTA YELLRG RPY | | |
| | | | | | | | | | I | | |
| | | | | | | | | | S+T EI++ F+ | | |
| | | | | | | | | | V Y S W + | | |
| | | | | | | | | | MVSLLKK+ K | | |
| SGK410 | 5 | 62 | FL | 1 | 261 | 289 | 557 | Complete | | 7018620_2 | 55 |
| SGK069 | 6 | 63 | Cat | 1 | 261 | 158 | 421 | Complete | | 11000283338966 | 26 |
| SGK110 | 7 | 64 | partial | 1 | 261 | 68 | 329 | Complete | | 11000284702500 | 35 |
| SGK053, CKLiK | 8 | 65 | FL | 1 | 261 | 23 | 279 | Complete | | 17000035790284 | 20 |
| SGK124 | 9 | 66 | FL | 1 | 261 | 73 | 315 | Complete | | 17000047891899 | 37 |
| SGK254, CAMKKa | 10 | 67 | FL | 1 | 261 | 128 | 417 | Complete | | 4454511_1_3 | 47 |
| SKG297, CaMKIb2 | 11 | 68 | FL | 1 | 261 | 15 | 270 | Complete | | 17000113122540 | 50 |
| SGK411, CaMKII delta2 | 12 | 69 | FL | 1 | 261 | 14 | 272 | Complete | | 2828765_1_3 | 56 |
| SGK027 | 13 | 70 | FL | 1 | 261 | 74 | 325 | Complete | | 17000029868654 | 9 |
| SGK046b | 14 | 71 | partial | 98 | 175 | 1 | 75 | Smith Waterman | | 11000284253087 | 16 |
| SGK046c | 15 | 72 | partial | 106 | 144 | 1 | 39 | Smith Waterman | | 11000284253087 | 17 |
| SGK089 | 16 | 73 | partial | 130 | 216 | 1 | 84 | Smith Waterman | | 11000283986586 | 32 |
| SGK133, N83965 | 17 | 74 | FL | 1 | 261 | 50 | 301 | Complete | | 6671894_1_1 | 40 |
| SGK004, MSK | 18 | 75 | FL | 1 | 261 | 27 | 281 | Complete | MVIMSEFSA PG | 11000257744647 | 3 |
| | | | | | | | | | GQGQQKPLRV | | |
| | | | | | | | | | GFYD+ERTLGK | | |
| | | | | | | | | | GNFAVVKLARH | | |
| | | | | | | | | | RVTKTQVAIKIID | | |
| | | | | | | | | | KTRLDSSNLEKI | | |
| | | | | | | | | | YREVQLMKLLN | | |
| | | | | | | | | | HP+IIKLYQVME | | |
| | | | | | | | | | TKDMLYIVTEFA | | |
| | | | | | | | | | KNGEMFDYLTS | | |
| | | | | | | | | | NGHLSENEARK | | |
| | | | | | | | | | KFWQILSAVEY | | |
| | | | | | | | | | CH+HHIVHRDL | | |
| | | | | | | | | | KTENLLLDGNM | | |
| | | | | | | | | | DIKLA | | |
| | | | | | | | | | DFGFGNFYK | | |
| | | | | | | | | | GEPLSTWCGS | | |
| | | | | | | | | | PPYAAPEVFEG | | |
| | | | | | | | | | KEYEGPQLDIW | | |
| | | | | | | | | | SLGVVLYVLVC | | |
| | | | | | | | | | GSLPFDGPNLP | | |
| | | | | | | | | | TLRQRVLEGRF RIPFFMSQDCE | | |
| | | | | | | | | | +LIRRMLVVDPA | | |
| | | | | | | | | | +RITIAQIRQHR | | |
| | | | | | | | | | WM+A+P L | | |
| SGK006 | 19 | 76 | Cat | 1 | 261 | 25 | 261 | Complete | +P + +S + F + | 11000257829454 | 4 |
| | | | | | | | | | K+VG YL+G + + | | |
| | | | | | | | | | +G FAKV EGL + | | |
| | | | | | | | | | GEK IK I | | |
| | | | | | | | | | KK+AK+D+YV | | |
| | | | | | | | | | KN++ E I | | |
| | | | | | | | | | Q++++PN +Q L | | |
| | | | | | | | | | LET+NSYY++M | | |
| | | | | | | | | | ELC | | |
| | | | | | | | | | GNLM++I+++K+ | | |
| | | | | | | | | | L E E + | | |
| | | | | | | | | | RQ++SA+EH+H | | |
| | | | | | | | | | G+VH+D + | | |
| | | | | | | | | | + + L N + G | | |
| | | | | | | | | | S + STQ | | |
| | | | | | | | | | GSPAYAAPELLA | | |
| | | | | | | | | | +KYGPK+DVW | | |
| | | | | | | | | | +G + | | |
| | | | | | | | | | MLTGTLPFT+E | | |
| | | | | | | | | | FS+ LYQKMV | | |
| | | | | | | | | | A++ + ++ | | |
| SGK180, SNRK | 20 | 77 | FL | 1 | 261 | 16 | 269 | Complete | | 17000057577785 | 43 |
| SGK386, MLCKs | 21 | 78 | FL | 1 | 261 | 301 | 556 | Complete | | 17000140438265 | 51 |
| SGK003 | 22 | 79 | FL | 1 | 261 | 17 | 299 | Complete | M ++SGSKAE | 11000257741254 | 2 |
| | | | | | | | | | +VGGKYKLVRKI | | |
| | | | | | | | | | GSGSFGD+YL I | | |
| | | | | | | | | | TNGE+VA+KLE | | |
| | | | | | | | | | SQK | | |
| | | | | | | | | | +HPQLLYESKLY | | |
| | | | | | | | | | ILQGGVGIPH+ | | |
| | | | | | | | | | WYGQEKD | | |
| | | | | | | | | | NVLVMDLLGPS | | |
| | | | | | | | | | LEDLFNFCSRR | | |
| | | | | | | | | | FTMKTVLMLAD | | |
| | | | | | | | | | QMISRIEYVHTK | | |
| | | | | | | | | | NF+HRDIKPDN | | |
| | | | | | | | | | FLMG | | |
| | | | | | | | | | GRHCNKLFLIDF | | |
| | | | | | | | | | GLAKKYRDNRT | | |
| | | | | | | | | | RQHIPYREDK+ | | |
| | | | | | | | | | L GT | | |
| | | | | | | | | | RYASINAHLGIE | | |
| | | | | | | | | | QSRRDDMESL | | |
| | | | | | | | | | GYV | | |
| | | | | | | | | | MYFNRTSLPW | | |
| | | | | | | | | | QGL+A | | |
| | | | | | | | | | TKKQKYEKISEK | | |
| | | | | | | | | | KMSTPVEVLCK | | |
| | | | | | | | | | GFPAEFAMYLN | | |
| SGK066 | 23 | 80 | Cat | 1 | 261 | 21 | 281 | Complete | | 11000283296349 | 24 |
| SGK041, NKIAMRE | 24 | 81 | FL | 1 | 261 | 4 | 286 | Complete | | 11000257953829 | 15 |
| SGK112 | 25 | 82 | FL | 1 | 261 | 4 | 304 | Complete | | 17000035915087 | 36 |
| SGK038, ERK7 | 26 | 83 | FL | 1 | 261 | 13 | 323 | Complete | | 17000030278391 | 13 |
| SGK158 | 27 | 84 | FL | 253 | 261 | 522 | 532 | Smith Waterman | | 11000283551497 | 42 |
| SGK429 | 28 | 85 | FL | 1 | 34 | 200 | 315 | Smith Waterman | | 5001549_3 | 60 |
| SGK152, SUDD | 29 | 86 | FL | 251 | 261 | 347 | 358 | Smith Waterman | | 11000258249295 | 41 |
| SGK077 | 30 | 87 | FL | 1 | 32 | 484 | 513 | Smith Waterman | | 11000283672012 | 29 |
| SGK093, Wnk3 | 31 | 88 | FL | 1 | 261 | 162 | 420 | Complete | | 11000284063441 | 33 |
| SGK074 | 32 | 89 | Cat | 1 | 261 | 6 | 342 | Complete | | 11000283476699 | 27 |
| SGK087 | 33 | 90 | FL | 1 | 261 | 506 | 703 | Complete | | 17000036171996 | 31 |
| SGK295, KIS | 34 | 91 | FL | 1 | 200 | 23 | 238 | Complete | | 17000113079883 | 49 |
| SGK419 | 35 | 92 | FL | 1 | 261 | 51 | 341 | Complete | | 5932598_100001_1 | 57 |
| SGK125, MYO3A | 36 | 93 | FL | 1 | 261 | 21 | 287 | Complete | | 17000078090910 | 38 |
| SGK445 | 37 | 94 | partial | 1 | 71 | 1 | 68 | Complete | | 4000001803382 | 61 |
| SGK127 | 38 | 95 | FL | 1 | 261 | 661 | 922 | Complete | | 17000062804843 | 39 |
| SGK009, ANKRD3 | 39 | 96 | FL | 1 | 261 | 22 | 276 | Complete | P | 11000258161587 | 6 |
| | | | | | | | | | +DLGTTDVQKK | | |
| | | | | | | | | | KLVDAIVSGDTS | | |
| | | | | | | | | | KLMKILQPQDV | | |
| | | | | | | | | | DLALDSGASLL | | |
| | | | | | | | | | HLAVEAGQEEC | | |
| | | | | | | | | | AKWLLLNNANP | | |
| | | | | | | | | | NLSNRRGSTPL | | |
| | | | | | | | | | HMAVERRVRG | | |
| | | | | | | | | | VVELLLARKISV | | |
| | | | | | | | | | NAKDEDQWTA | | |
| | | | | | | | | | LHFAAQNGDES | | |
| | | | | | | | | | STRLLLEKNASV | | |
| | | | | | | | | | NEVDFEGRTPM | | |
| | | | | | | | | | HVACQHGQENI | | |
| | | | | | | | | | VRILLRRGVDVS | | |
| | | | | | | | | | LQGKDAWLPLH | | |
| | | | | | | | | | YAAWQGHLPIV | | |
| | | | | | | | | | KLLAKQPGVSV | | |
| | | | | | | | | | NAQTLDGRTPL | | |
| | | | | | | | | | HLAAQRGHYRV | | |
| | | | | | | | | | ARILIDLCSDVN | | |
| | | | | | | | | | VCSLLAQTPLH | | |
| | | | | | | | | | VAAETGHTSTA | | |
| | | | | | | | | | RLLLHRGAGKE | | |
| | | | | | | | | | A+TSDGYTALH | | |
| | | | | | | | | | LAARNGHLATV | | |
| SGK421, STK22A, TSK1 | 40 | 97 | FL | 1 | 261 | 12 | 272 | Complete | | 6759077_1_4 | 59 |
| SGK047 | 41 | 98 | partial | 232 | 261 | 1 | 31 | Smith Waterman | | 11000258262374 | 18 |
| SGK196 | 42 | 99 | FL | 1 | 88 | 81 | 160 | Complete | | 17000062537825 | 46 |
| SGK396 | 43 | 100 | FL | 1 | 261 | 186 | 425 | Complete | | 5630059_1_4 | 53 |
| SGK279, PKN | 44 | 101 | FL | 1 | 261 | 53 | 313 | Complete | | 17000097259742 | 48 |
| SGK037 | 45 | 102 | FL | 1 | 261 | 4 | 259 | Complete | | 17000030265658 | 12 |
| SGK060 | 46 | 103 | FL | 1 | 261 | 29 | 287 | Complete | | 17000036897142 | 22 |
| SGK080 | 47 | 104 | FL | 1 | 261 | 8 | 269 | Complete | | 17000048119006 | 30 |
| SGK002 | 48 | 105 | FL | 1 | 261 | 71 | 368 | Complete | MLARRKP+LPA | 11000257702671 | 1 |
| | | | | | | | | | LTINPTIAEGPS | | |
| | | | | | | | | | PTSEGASEANL | | |
| | | | | | | | | | VDLQKKLEEL+L | | |
| | | | | | | | | | DEQQ | | |
| | | | | | | | | | KRLEAFLTQKA | | |
| | | | | | | | | | KVGELKDDDFE | | |
| | | | | | | | | | R SEL | | |
| | | | | | | | | | AGNGGVVTK | | |
| | | | | | | | | | +HRPSGLIMAR | | |
| | | | | | | | | | KLIHLEIKPA+R | | |
| | | | | | | | | | NQIIRE | | |
| | | | | | | | | | QVLHECNSPYI | | |
| | | | | | | | | | VGFYGAFY D | | |
| | | | | | | | | | EISICMEHMDG | | |
| | | | | | | | | | GSLDQ | | |
| | | | | | | | | | LKEAKRIPEDIL | | |
| | | | | | | | | | GKVSIAVLRGLA | | |
| | | | | | | | | | YLREKHQIMHR | | |
| | | | | | | | | | +VKPSNILVNSR | | |
| | | | | | | | | | GEIKLCDFGVS GQLIDSMANSF | | |
| | | | | | | | | | VGTRSYM+PER | | |
| | | | | | | | | | LQGTHYSVQS | | |
| | | | | | | | | | IWSM LSLVELAI | | |
| | | | | | | | | | RYPIPPPDAKEL | | |
| | | | | | | | | | EA FG+PVVD | | |
| SGK058 | 49 | 106 | Cat | 1 | 261 | 11 | 274 | Complete | | 17000036877196 | 21 |
| SGK103 | 50 | 107 | partial | 125 | 148 | 1 | 24 | Smith Waterman | | 11000284272557 | 34 |
| SGK035 | 51 | 108 | partial | 1 | 261 | 73 | 324 | Complete | PPVIAP PDH | 17000030169905 | 11 |
| | | | | | | | | | KSIYTR+VIDPV | | |
| | | | | | | | | | APVGDS+VDG | | |
| | | | | | | | | | AKSLDKQKKK | | |
| | | | | | | | | | KMTDEEIMEKL | | |
| | | | | | | | | | RTIVSIG | | |
| | | | | | | | | | +KKYTRYEKIG | | |
| | | | | | | | | | QGASGTVFTAT | | |
| | | | | | | | | | DVALGQ+VAIK | | |
| | | | | | | | | | QINLQKQPKKE | | |
| | | | | | | | | | LIINEILVMKELK | | |
| | | | | | | | | | NPNIVNFLDSYL | | |
| | | | | | | | | | VGDELFVV+EY | | |
| | | | | | | | | | LA | | |
| | | | | | | | | | GSLTDVVTETC | | |
| | | | | | | | | | MDEAQIAAVCRE | | |
| | | | | | | | | | LQALEFLHANQ | | |
| | | | | | | | | | VIHRDIKSD+VL | | |
| | | | | | | | | | LGMEGSVKLTD | | |
| | | | | | | | | | FGFCAQITPEQ | | |
| | | | | | | | | | SKRST+V | | |
| | | | | | | | | | TP+WMAPEVVT | | |
| | | | | | | | | | RKAYGPKV | | |
| | | | | | | | | | +WSLGIMA | | |
| | | | | | | | | | EMVEGEPPYLN | | |
| | | | | | | | | | ENPLRAL | | |
| SGK075 | 52 | 109 | Cat | 1 | 261 | 15 | 281 | Complete | | 11000283492249 | 28 |
| SGK188, EphA9 | 53 | 110 | FL | 1 | 261 | 645 | 899 | Complete | C+ G P + EV | 17000057739161 | 45 |
| | | | | | | | | | LLDSK Q | | |
| | | | | | | | | | ELGW + | | |
| | | | | | | | | | PS+GWEEISGV | | |
| | | | | | | | | | DEH | | |
| | | | | | | | | | PIRTYQVCNV++ | | |
| | | | | | | | | | +Q+NWL+T W+R | | |
| | | | | | | | | | Q+I+VEL+FTLR | | |
| | | | | | | | | | DC+SIP | | |
| | | | | | | | | | GTCKETFN+YY | | |
| | | | | | | | | | +E++ + R + | | |
| | | | | | | | | | KIDTIAADESFT | | |
| | | | | | | | | | Q DLG+R | | |
| | | | | | | | | | +KLNTE+RE+G | | |
| | | | | | | | | | P++++GF+LAFQ | | |
| | | | | | | | | | DVGACVALVSV | | |
| | | | | | | | | | RVY+K+C TV+ | | |
| | | | | | | | | | LA FP T + | | |
| | | | | | | | | | +LVEV G+CV | | |
| | | | | | | | | | +S+ E | | |
| | | | | | | | | | PPRM+C | | |
| | | | | | | | | | +GEWLVP+G+C | | |
| | | | | | | | | | +C+AG++ERG | | |
| | | | | | | | | | IC+AC PGFYK S | | |
| | | | | | | | | | C+ CP HS E+ S | | |
| | | | | | | | | | C C+++Y R+ | | |
| SGK040 | 54 | 111 | FL | 1 | 261 | 394 | 646 | Complete | | 11000257912897 | 14 |
| SGK390 | 55 | 112 | FL | 1 | 13 | 383 | 395 | Smith Waterman | | 17000112562523 | 52 |
| SGK007 | 56 | 113 | Cat | 166 | 261 | 613 | 716 | Smith Waterman | | 11000257855312 | 5 |
| SGK050 | 57 | 114 | partial | 51 | 89 | 1 | 37 | Smith Waterman | | 17000035767558 | 19 |

Table 4 describes the results of Smith Waterman similarity searches (Matrix: Pam100; gap open/extension penalties 12/2) of the amino acid sequences against the NCBI database of non-redundant protein sequences (http://www.ncbi.nlm.nih.gov/Entrez/protein.html). The column headings are: “Gene Name”, “ID#na”, “ID#aa”, “FL/Cat”, “Superfamily”, “Group”, “Family”, “Pscore”, “aa_length”, “aa_ID_match”, “% Identity”, “% Similar”, “ACC#_nraa_match”, and “Description”. The contents of the first 8 columns (i.e.,. “Gene Name”, “ID#na”, “ID#aa”, “FL/Cat”, “Serial #”, “Superfamily”, “Group”, “Family”) are as described above for Table 1. “Pscore” refers to the Smith Waterman probability score. This number approximates the chance that the alignment occurred by chance. Thus, a very low number, such as 2.10E-64, indicates that there is a very significant match between the query and the database target. “aa_length” refers to the length of the protein in amino acids. “aa_ID_match” indicates the number of amino acids that were identical in the alignment. “% Identity” lists the percent of nucleotides that were identical over the aligned region. “% Similarity” lists the percent of amino acids that were similar over the alignment. “ACC#nraa_match” lists the accession number of the most similar protein in the NCBI database of non-redundant proteins. “Description” contains the name of the most similar protein in the NCBI database of non-redundant proteins.

TABLE 4
Smith Waterman
413406_1.xls

| | | | | Super- | | | | | | |
|---|---|---|---|---|---|---|---|---|---|---|
| Gene Name | ID#na | ID#aa | FL/Cat | family | Group | Family | Pscore | aa_length | aa_ID_match | % Identity |
| SGK187, CRIK | 1 | 58 | FL | PK | AGC | DMPK | 0 | 2053 | 1970 | 96 |
| SGK064, GRK7 | 2 | 59 | FL | PK | AGC | GRK | 0 | 553 | 466 | 84 |
| SGK409, KIAA0303 | 3 | 60 | FL | PK | AGC | MAST | 0 | 2523 | 2137 | 100 |
| SGK021 | 4 | 61 | Cat | PK | AGC | Mo3C11.1_ce | 6.00E−138 | 327 | 203 | 77 |
| SGK410 | 5 | 62 | FL | PK | AGC | PKC | 0 | 631 | 555 | 95 |
| SGK069 | 6 | 63 | Cat | PK | AGC | Unique | 2.10E−64 | 572 | 116 | 42 |
| SGK110 | 7 | 64 | partial | PK | AGC | Unique | 1.10E−65 | 373 | 112 | 40 |
| SGK053, CKLiK | 8 | 65 | FL | PK | CAMK | AMPK | 1.60E−245 | 357 | 357 | 100 |
| SGK124 | 9 | 66 | FL | PK | CAMK | AMPK | 5.10E−252 | 358 | 358 | 100 |
| SGK254, CAMKKa | 10 | 67 | FL | PK | CAMK | CAMK | 2.0e−323 | 513 | 470 | 91 |
| SGK297, CaMKIb2 | 11 | 68 | FL | PK | CAMK | CAMK | 1.40E−236 | 343 | 341 | 99 |
| SGK411, CaMKII | 12 | 69 | FL | PK | CAMK | CAMK | 0 | 499 | 499 | 100 |
| delta2 | | | | | | | | | | |
| SGK027 | 13 | 70 | FL | PK | CAMK | EMK | 7.70E−101 | 436 | 147 | 56 |
| SGK046b | 14 | 71 | partial | PK | CAMK | EMK | 1.40E−14 | 75 | 35 | 47 |
| SGK046c | 15 | 72 | partial | PK | CAMK | EMK | 6.40E−07 | 39 | 21 | 54 |
| SGK089 | 16 | 73 | partial | PK | CAMK | EMK | 2.40E−19 | 84 | 39 | 46 |
| SGK133, N83965 | 17 | 74 | FL | PK | CAMK | EMK | 8.2e−318 | 794 | 481 | 75 |
| SGK004, MSK | 18 | 75 | FL | PK | CAMK | EMK-1 | 0 | 786 | 784 | 100 |
| SGK006 | 19 | 76 | Cat | PK | CAMK | EMK-1 | 1.50E−62 | 262 | 121 | 45 |
| SGK180, SNRK | 20 | 77 | FL | PK | CAMK | EMK-1 | 0 | 765 | 757 | 99 |
| SGK386, MLCKs | 21 | 78 | FL | PK | CAMK | MLCK | 0 | 612 | 596 | 97 |
| SGK003 | 22 | 79 | FL | PK | CKI | CKI | 1.40E−215 | 337 | 304 | 91 |
| SGK066 | 23 | 80 | Cat | PK | CKI | CKI | 5.40E−106 | 400 | 166 | 53 |
| SGK041, | 24 | 81 | FL | PK | CMGC | CDK | 1.0e−319 | 591 | 454 | 99 |
| NKIAMRE | | | | | | | | | | |
| SGK112 | 25 | 82 | FL | PK | CMGC | CDK | 8.70E−151 | 360 | 224 | 61 |
| SGK038, ERK7 | 26 | 83 | FL | PK | CMGC | MAPK | 1.80E−105 | 371 | 167 | 59 |
| SGK158 | 27 | 84 | FL | PK | Microbial | ABC1 | 3.30E−257 | 751 | 368 | 100 |
| | | | | | PK | | | | | |
| SGK429 | 28 | 85 | FL | PK | Microbial | ABC1 | 6.00E−122 | 626 | 191 | 100 |
| | | | | | PK | | | | | |
| SGK152, SUDD | 29 | 86 | FL | PK | Microbial | RI01 | 0 | 519 | 519 | 100 |
| | | | | | PK | | | | | |
| SGK077 | 30 | 87 | FL | PK | Other | C26C2_ce | 8.50E−306 | 798 | 492 | 62 |
| SGK093, Wnk3 | 31 | 88 | FL | PK | Other | C26C2_ce | 2.10E−203 | 1513 | 321 | 60 |
| SGK074 | 32 | 89 | Cat | PK | Other | DYRK | 2.00E−107 | 355 | 176 | 50 |
| SGK087 | 33 | 90 | FL | PK | Other | DYRK | 6.20E−183 | 703 | 267 | 100 |
| SGK295, KIS | 34 | 91 | FL | PK | Other | DYRK | 1.10E−292 | 419 | 415 | 99 |
| SGK419 | 35 | 92 | FL | PK | Other | NAK | 5.00E−252 | 661 | 355 | 100 |
| SGK125, MYO3A | 36 | 93 | FL | PK | Other | NinaC | 0 | 1615 | 1615 | 100 |
| SGK445 | 37 | 94 | partial | PK | Other | PLK | 9.90E−25 | 68 | 47 | 65 |
| SGK127 | 38 | 95 | FL | PK | Other | RAF | 8.60E−254 | 945 | 446 | 47 |
| SGK009, ANKRD3 | 39 | 96 | FL | PK | Other | RIP | 0 | 784 | 784 | 100 |
| SGK421, STK22A, | 40 | 97 | FL | PK | Other | STK22A | 6.40E−209 | 367 | 307 | 84 |
| TSK1 | | | | | | | | | | |
| SGK047 | 41 | 98 | partial | PK | Other | STKR | 0.265466 | 31 | 11 | 46 |
| SGK196 | 42 | 99 | FL | PK | Other | Unique | 2.70E−248 | 350 | 350 | 100 |
| SGK396 | 43 | 100 | FL | PK | Other | Unique | 3.00E−09 | 472 | 34 | 28 |
| SGK279, PKN | 44 | 101 | FL | PK | Other | YWY3_ce | 4.10E−276 | 424 | 400 | 94 |
| SGK037 | 45 | 102 | FL | PK | STE | NEK | 4.10E−56 | 649 | 176 | 65 |
| SGK060 | 46 | 103 | FL | PK | STE | NEK | 1.3e−322 | 645 | 463 | 100 |
| SGK080 | 47 | 104 | FL | PK | STE | NEK | 3.80E−266 | 446 | 404 | 90 |
| SGK002 | 48 | 105 | FL | PK | STE | STE11 | 3.10E−248 | 399 | 369 | 92 |
| SGK058 | 49 | 106 | Cat | PK | STE | STE11 | 3.00E−116 | 278 | 167 | 99 |
| SGK103 | 50 | 107 | partial | PK | STE | STE11 | 0.008942 | 28 | 16 | 57 |
| SGK035 | 51 | 108 | partial | PK | STE | STE20 | 7.60E−212 | 348 | 318 | 92 |
| SGK075 | 52 | 109 | Cat | PK | STE | STE20 | 4.00E−158 | 1106 | 227 | 67 |
| SGK188, EphA9 | 53 | 110 | FL | PK | TK | RTK-11 | 0 | 1009 | 538 | 54 |
| SGK040 | 54 | 111 | FL | PK | TK | Unique | 4.50E−26 | 909 | 76 | 30 |
| SGK390 | 55 | 112 | FL | PK-like | DAG kin | DAG kin | 0 | 1164 | 1063 | 91 |
| SGK007 | 56 | 113 | Cat | PK-like | GCyc | GCyc | 5.20E−265 | 884 | 499 | 51 |
| SGK050 | 57 | 114 | partial | PK-like | GCyc | GCyc | 1.30E−10 | 48 | 23 | 48 |

| Gene Name | % Similar | ACC#_nraa_match | Description | Genomic_ID | Comparison |
|---|---|---|---|---|---|
| SGK187, CRIK | 98 | AAC72823.1 | Rho/rac-interacting | 4926912_1_1 | MLKFKYG RNP +A |
| | | | citron kinase [Mus | | A+EPIASRASRLNLFFQGKPP |
| | | | musculus] | | MTQQQMS LSREG+LDALF |
| | | | | | LFEECSQPALMK+KHVS+FV + |
| | | | | | YSDTIAEL+ELQPSA+DFEVRSLV |
| | | | | | GCGHFAEVQVVREKATGD+YAM |
| | | | | | K+MKKKALLAQEQVSFFEEERNI |
| | | | | | LSRSTSPWIPQLQYAFQQKN+LY |
| | | | | | LVMEYQPGGD |
| | | | | | LSLLNRYEDQLDE++IQFYLAELIL |
| | | | | | AVHSVH |
| | | | | | MGYVHRDIKPENIL+DRTG |
| | | | | | IKLVDFGSAAKMNSNK V+A |
| SGK064, GRK7 | 91 | AAC95001.1 | G protein-coupled | 11000284009826 | x+DMG LDNLIANTAYLQARK +D |
| | | | receptor kinase | | DS+ELQRRRRSLALPG |
| | | | GRK7 | | QGCAELRQ LS |
| | | | [Spermophilus | | +FHSLCEQQPIGRRLFRDFLATV |
| | | | tridecemlinealus] | | P + +A FLEOVQNWELAEEGP K |
| | | | | | S LQ L ATCA P PQ FLSQ |
| | | | | | +ATKC+AA+T+EER V AKAE |
| | | | | | M+FLQEQPF+DF+ S |
| | | | | | FYD+FLQWKLFEMQPVSDKYFT |
| | | | | | EFRVLGKGGFGEVCAVQV+NTG |
| | | | | | KMYACKKLDKKRLKKKGGEKMA |
| | | | | | LLEKEILEKV+SPFIVSLAYAFESK |
| | | | | | THLCLVMSLMNGGDLKFHIYNVG |
| | | | | | TRGL MSRVIFY+AQ+ CG+LHLH |
| | | | | | LGIVYRD+KPEN |
| | | | | | LLDDLGNCRLSDLGLAVE++ |
| | | | | | KPITQRAGTNGYMAPEILM+K |
| | | | | | SYSYPVDWFAMGCSIYEMVAGR |
| | | | | | TPFKD+KEKVSKEDLK+RT++DE |
| | | | | | V F H+NFTEE |
| | | | | | KDICRLFLAKKPEQRL |
| | | | | | SREK+DDPRKH |
| | | | | | FF+T+NFPRLEAGL+EPPFVPDP |
| | | | | | SVVYAKD+ |
| | | | | | EIDDFSEVRGVEFDDKDKQFF+ |
| | | | | | F+TGAVP+AWQEEIIETGLFEELN |
| | | | | | DPNRP+G +G+SSKSGVCLLL |
| SGK409, KIAA0303 | 100 | BAA20762.1 | KIAA0303 [Homo | 6671897_1_1 | EFDPEEFYYLLEAAEGHAKEGQ |
| | | | sapiens] | | GIKTDIPRYIISQLGLNKDPLEEMA |
| | | | | | HLGNYDSGTAETPETDESVSSS |
| | | | | | NASLKLRRKPRESDFETIKLISNG |
| | | | | | AYGAVYFVRHKESRORFAMKKI |
| | | | | | KQNLILRNQIQQAFVERDILTFAE |
| | | | | | NPFVVSMYCSFETRRHLCMVME |
| | | | | | YVEGGDCATLMKNMGPLPVDMA |
| | | | | | RMYFAETVLALEYLHNYGIVHRD |
| | | | | | LKPDNLLVTSMGHIKLTDFGLSKV |
| | | | | | GLMSMTTNLYEGHIEKDAREFLDK |
| SGK021 | 84 | CAB76566.1 | Serine/theronine | 17000028181153 | MG N S |
| | | | protein kinase | | KPPVFDENE+VNFDHF+ILRAIGK |
| | | | [Mus musculus] | | G FGKVCIVQK |
| | | | | | DTKKMYAMKYMNKQKCVER+EV |
| | | | | | RNVF+ELQIMQGLEHPFLVNLWY |
| | | | | | SFQDEEDMFMVVDLLLGGDLRY |
| | | | | | HLQQNVHF E TVKL+ICEL |
| | | | | | +AL+YLQ |
| | | | | | IIHRD+KPDNILLDEHGHVHITDFN |
| | | | | | IA +L + ++MAGTKPYMAPE+F |
| | | | | | G GYS+ VDWWSLGVTAYELLRG |
| | | | | | RPY I S+T EI++ F+ V Y S W + M |
| SGK410 | 96 | NP_002731.1 | Protein kinase C, | 7018620_2 | MSH VAGG SGDHSHQV |
| | | | iota [Homo | | VKAYYRGDIMITHFEPS+SFEGLN |
| | | | sapiens] | | VRDMCSFDNEQLFTMKWIDEEG |
| | | | | | DPCTVSSQLELEEAF |
| | | | | | LYELNKDSELLIHVFPCVPERPG |
| | | | | | MPCP EDKSIYRRGAR |
| | | | | | WRKLYCANGHTFQAK FNRRA |
| | | | | | CAICTDRIWGLG |
| | | | | | QGYKCINCKLLVHKKCKKL+TIEC |
| | | | | | GRHSLP |
| | | | | | EP+MPMDQSSMHSDHAQTVIPY |
| | | | | | NPSSHESLDQVGEEKEAMNTRE |
| | | | | | SGKASSSLGLQDFDLL+VIGRGS |
| SGK069 | 58 | BAA36362.1 | Serin/threonine | 11000283338966 | EDM L+ +TL ++V + YE VR |
| | | | protein kinase | | LG+G YG+V LV ++ GT +ALK + K |
| | | | [Rattus norvegicus] | | +T L+ FL E + SL + I+ + + E+ |
| | | | | | Y F E GDL I P+VGLP+ V RC |
| | | | | | QL AL+++H+R |
| | | | | | LV+RD+KPENVL+ D CRR KL |
| | | | | | DFG TR G ++ IPYTAPE+C |
| | | | | | +G + +D WA GVL+FC+LTG |
| | | | | | FPW+ + AD F+E+F+ WQ P PW |
| SGK110 | 58 | S71887 | pk9.7 gastrula- | 11000284702500 | D YH+I+KLG G++ VLLA+ G |
| | | | specific PK | | +VALK +R+D ++F+ E + +S |
| | | | [Xenopus laevis] | | +PG+++T ++T YF F QE AP G L |
| | | | | | +++ E G+PE +VKR Q+ ALDF+H |
| | | | | | R LVH D+KPDNVL+ D C + L D G |
| | | | | | T+ GS P+ +P PPELC L P LL |
| | | | | | ++D W G+LLF A T FPW+ A+ N |
| | | | | | +++ F W + P P W++F A+A+ |
| | | | | | LL P +R |
| SGK053, CKLiK | 100 | NP_065130.1 | CamKI-like protein | 17000035790284 | MARENGESSSSWKKQAEOIKKIF |
| | | | kinase [Homo | | EFKETLGTGAFSEVVLAEEKATG |
| | | | sapiens] | | KLFAVKCIPKKALKGKESSIENEIA |
| | | | | | VLRKIKHENIVALEDIYESPNHLYL |
| | | | | | VMQLVSGGELFDRIVEKGFYTEK |
| | | | | | DASTLIRQVLDAVYYLHRMGIVH |
| | | | | | RDLKPENLLYYSQDEESKIMISDF |
| | | | | | GLSKMEGKGDVMSTACGTPGYV |
| | | | | | APEVLAQKPYSKAVOCWSIGVIA |
| | | | | | YILLCGYPPFYDENDSKLFEQILK |
| | | | | | AEYEFDSPYWDOISOSAKDF |
| SGK124 | 100 | CAB81634.1 | Novel protein | 17000047891899 | MRATPLAAPAGSLSRKKRLELDO |
| | | | kinase [Homo | | NLDTERPVQKRARSGPQPRLPP |
| | | | sapiens] | | CLLPLSPPTAPDRATAVATASRL |
| | | | | | GPYVLLEPEEGGRAYQALHCPT |
| | | | | | GTEYTCKVYPVQEALAVLEPYAR |
| | | | | | LPPHKHVARPTEVLAGTQLLYAF |
| | | | | | FTRTHGDMHSLVRSRHRIPEPEA |
| | | | | | AVLFRQMATALAHCHQHGLVLR |
| | | | | | DLKLCRFVFADRERKKLVLENLE |
| | | | | | DSCVLTGPDDSLWDKHACPAYV |
| | | | | | GPEILSSRASYSGKAADVWSLGV |
| | | | | | ALFTML |
| SGK254, CAMKKa | 94 | A57156 | Ca2+/calmodulin- | 4454511_1_3 | ME PAVCCQDPRAELVERVAAI |
| | | | dep. PK IV [Rattus | | V HLEEA+ GPEP NGVDP |
| | | | norvegicus] | | PRARAASVIPGS SR P |
| | | | | | RPSLSARK SLQERPA S LEAQ |
| | | | | | GPY+TGPASH+SPRAWRRPTIES |
| | | | | | HHVAISD |
| | | | | | EDCVQLNQYKLQSEIGK |
| | | | | | GAYGVVRLAYNE |
| | | | | | EDRHYAMKVLSKKKLLKQYGFP |
| | | | | | RRPPPRGSQA |
| | | | | | QGGPAKQLLPLERVYQEIAILKKL |
| | | | | | DHVNVVKLIEVLDDPAEDNLYLV |
| | | | | | DLLRKGPVMEVPCOKP |
| SGK297, CaMKIb2 | 100 | AAF74509.1 | Ca2+/Calmodulin- | 17000113122540 | MLLLKKHTEDISSVYEIRERLGSG |
| | | | dependent protein | | AFSEVVLAQERGSAHLVALKCIP |
| | | | kinase I [Homo | | KKALRGKEALVENEIAVLRRISHP |
| | | | sapiens] | | NIVALEDVHESPSHLYLAMELVT |
| | | | | | GGELFORIMERGSYTEKDASHLV |
| | | | | | GQVLGAVSYLHSLGIVHRDLKPE |
| | | | | | NLLYATPFEDSKIMVSDFGLSKIQ |
| | | | | | AGNMLGTACGTPGYVAPELLEQ |
| | | | | | KPYGKAVDVWALGVISYILLCGY |
| | | | | | PPFYDESDPELFSQILRASYEFD |
| | | | | | SPFW DISESAKDFIRHLLERDP |
| SGK411, CaMKII | 100 | AAD20442.1 | Multifunctional | 2828765_1_3 | MASTTTCTRFTDEYQLFEELGKG |
| delta2 | | | CAMK II delta2 | | AFSVVRRCMKIPTGQGYAAKIINT |
| | | | [Homo sapiens] | | KKLSARDHQKLEREARICRLLKH |
| | | | | | PNIVRLHDSISEEGFHYLVFDLVT |
| | | | | | GGELFEDIVAREYYSEADASHCI |
| | | | | | QQILESVNHCHLNGIVHRDLKPE |
| | | | | | NLLLASKSKGAAVKLADFGLAIEV |
| | | | | | QGDQQAWFGFAGTPGYLSPEVL |
| | | | | | RKDPYGKPVDMWACGVILYILLV |
| | | | | | GYPPFWDEDQHRLYQQIKAGAY |
| | | | | | DFPSPEWDTVTPEAKDLINKMLTI |
| SGK027 | 76 | T22427 | F49C5.4 - | 17000029868654 | ++ LG+RIGFYR+ |
| | | | [Caenorhabditis | | E+G+GNFS+VKLG+H |
| | | | elegans] | | LTKEKVA+KI+DK K+D K |
| | | | | | Q+LLSREI |
| | | | | | +ME+++HPNI++L+EVVETL+++HL |
| | | | | | V+EYA GGEL+ + GKL+E ++K |
| | | | | | +F+QIVSAV HMH |
| | | | | | I+HRD+KAENV ++S VK+ |
| | | | | | DFGFS + + +ML |
| | | | | | TFCGSPPYAAPELF+D Y G |
| | | | | | VD+WALGVLL+FM+ G PF+AETV |
| | | | | | +K I G Y +P +VS LI+ +L+ |
| | | | | | +R ID |
| SGK046b | 61 | AAC33487.1 | R31237_1, partial | 11000284253087 | x+E+EA+ F QI+ A+ YC KN+ H |
| | | | CDS [Homo | | D+ + +LL E NIKI DFGFS F + |
| | | | sapiens] | | FCG PY A ELF |
| SGK046c | 67 | AAC33487.1 | R31237_1, partial | 11000284253087 | FRQI+S + YC KNI H +LK + L+ E |
| | | | CDS [Homo | | NIKI DF |
| | | | sapiens] | | |
| SGK089 | 71 | AAA97437.1 | Serine/threonine | 11000283986586 | N+ D++ NIKIA++GFS+TF+ + + |
| | | | kinase | | TF G+ PYA PELF G Y P |
| | | | [Caenorhabditis | | ++VWSLG+I+ +V+G+LPF QN |
| | | | elegans] | | K++ ++ |
| SGK133, N83965 | 83 | CAA07196.1 | Putative | 6671894_1_1 | LIEHPHVLKLHDVYENKKYLYLVL |
| | | | serine/threonine | | EHVSGGELFDYLVKKGRLTPKEA |
| | | | protein kinase | | RKFFRQI+SALDFCHS+SICHRDL |
| | | | [Homo sapiens] | | KPENLLLDEKNNIRIADFGMASLQ |
| | | | | | VGDSLLETSCGSPHYACPEVI+G |
| | | | | | EKYDGR+AD+WSCGVILFALLVG |
| | | | | | ALPFDDDNLRQLLEKVKRGVFH |
| | | | | | PHFIPPDCQSLLRGM EV+ |
| | | | | | +RL+LE IQKH WY+GGK+EP+P |
| | | | | | +P P R+V +RSLPS |
| | | | | | ++DPDVL+SM SLGCFRDR +L |
| | | | | | ++L S |
| SGK004, MSK | 100 | P57059 | SNF1LK [Homo | 11000257744647 | MVIMSEFSADPAGQGQGQQKPL |
| | | | sapiens] | | RVGFYDIERTLGKGNFAVVKLAR |
| | | | | | HRVTKTQVAIKIIDKTRLDSSNLE |
| | | | | | KIYREVQLMKLLNHPHIIKLYQVM |
| | | | | | ETKDMLYIVTEFAKNGEMFDYLT |
| | | | | | SNGHLSENEARKKFWQILSAVEY |
| | | | | | CHDHHIVHRDLKTENLLLDGNMD |
| | | | | | IKLAGTEDFGFGNFYKSGEPLST |
| | | | | | WCGSPPYAAPEVFEGKEYEGPQ |
| | | | | | LDIWSLGVVLYVLVCGSLPFDGP |
| | | | | | NLPTLRQRVLEGRFRIPFFMSQD |
| | | | | | CE |
| SGK006 | 66 | NP_056570.1 | Hormonally | 11000257829454 | +P + +S+ F + K+VG YL+G + + |
| | | | upregulated Neu- | | +G FAKV EGL + GEK IK I |
| | | | associated kinase | | KK+AK+D+YV KN++ E I |
| | | | [Mus musculus] | | Q++++PN +Q L |
| | | | | | LET+NSYY++MELC |
| | | | | | GNLM++I+++K+L E E + |
| | | | | | RQ++SA+EH+H G+VH+D + |
| | | | | | + + L N + G S + STQ |
| | | | | | GSPAYAAPELLA +KYGPK+DVW |
| | | | | | +G + MLTGTLPFT+E FS+ |
| | | | | | LYQKMV A++ + ++ |
| SGK180, SNRK | 99 | AAF86944.1 | HSNFRK [Homo | 17000057577785 | MAGFKRGYDGKIAGLYDLDKTLG |
| | | | sapiens] | | RGHFA |
| | | | | | +RHVFTGEKVAVKVIDKTKLDTLA |
| | | | | | TGHLFQEVRCMKLVQHPNIVRLY |
| | | | | | EVIDTQTKLYLILELGD |
| | | | | | GDMFDYIMKHEEGLNEDL |
| | | | | | KKYFAQIVHAISYCHKLHVVHRDL |
| | | | | | KPENVVFFEKQGLVKLTDFGFSN |
| | | | | | KFQPGKKLTTSCGSLAYSAPEILL |
| | | | | | GDEYDAPAVDIWSLGVILFMLVC |
| | | | | | GQPPFQEANDSETLTMIMDCKY |
| | | | | | TVPSHVSKECKDLITRMLQRDPK |
| SGK386, MLCKs | 97 | CAC10006.1 | MYLK (myosin, | 17000140438265 | MATENGAVELGIQNPST |
| | | | light polypeptide | | DKAPKGPTGERPLAAGKDPGPP |
| | | | kinase) [Homo | | DPKKAPDPPTLKKDAKAPASEKG |
| | | | sapiens] | | DGTLAQPSTSSQGPKGEGDRG |
| | | | | | GGPAEGSAGPPAALPQQTATPE |
| | | | | | TSVKKPKAEQGASGSQDPGKPR |
| | | | | | VGKKAAEGQAAARRGSPAFLHS |
| | | | | | PSCPAIISSSEKLLAKKPPSEASE |
| | | | | | LTFEGVPMTHSPTDPRPAKAEE |
| | | | | | GKNILAESQKEVGEKTPGQAGQ |
| | | | | | AKMQGDTSRGIEFQAVPSEKSE |
| SGK003 | 95 | NP_001883.2 | Casein kinase 1, | 11000257741254 | M ++SGSKAE |
| | | | alpha 1 [Homo | | +VGGKYKLVRKIGSGSFGD+YL I |
| | | | sapiens] | | TNGE+VA+KLESQK |
| | | | | | +HPQLLYESKLY ILQGGVGIPH+ |
| | | | | | WYGQEKD |
| | | | | | NVLVMDLLGPSLEDLFNFCSRRF |
| | | | | | TMKTVLMLADQMISRIEYVHTKN |
| | | | | | F+HRDIKPDNFLMG |
| | | | | | GRHCNKLFLIDFGLAKKYRDNRT |
| | | | | | RQHIPYREDK+L GT |
| | | | | | RYASINAHLGIEQSRRDDMESLG |
| | | | | | YV MYFNRTSLPWQGL+A |
| | | | | | TKKQKYEKISEKKMSTPVEVLCK |
| | | | | | GFPAE |
| SGK066 | 68 | T24262 | R90.1 | 11000283296349 | +IL VG +++ERWK+ |
| | | | [Caenorhabditis | | KIGGGGFGEIY+A D+ E |
| | | | elegans] | | VA+KVES++ |
| | | | | | KQVLKMEVAVL++LQG +Y |
| | | | | | GR+ ++ MS G+NLADLRR + F |
| | | | | | +ST +R+G QIL I |
| | | | | | IHS+GFLHRD+KPSNFAMGR T |
| | | | | | R YMLDFGLARQ+ N+ G++R |
| | | | | | PR+ AGFRGTVRYA++ |
| | | | | | AH+N+EMGR DDLWSLFYML |
| | | | | | EF+ GQLPWRKIKDK++VG +KE |
| | | | | | D ++L P E +F+ H+ |
| SGK041, | 100 | NP_057592.1 | NKIAMRE [Homo | 11000257953829 | MEMYETLGKVGEGSYGTVMKCK |
| NKIAMRE | | | sapiens] | | HKNTGQIVAIKIFYERPEQSVNKI |
| | | | | | AMREIKFLKQFHHENLVNLIEVFR |
| | | | | | QKKKIHLVFEFIDHTVLDELQHYC |
| | | | | | HGLESKRLRKYLFQILRAIDYLHS |
| | | | | | NNIIHRDIKPENILVSQSGITKLCD |
| | | | | | FGFARTLAAPGDIYTDYVATRWY |
| | | | | | RAPELVLKDTSYGKPVDIWALGC |
| | | | | | MIIEMATGNPYLPSSSDLDLLHKI |
| | | | | | VLKVGNLSPHLQNIFSKSPIFAGV |
| | | | | | VLPQVQHPKNARKKYPKL |
| SGK112 | 74 | NP_004187.1 | CDC2-related | 17000035915087 | MEKYEK+ K |
| | | | kinase [Homo | | GEGSYGVVFKCRN+ |
| | | | sapiens] | | +GQ+VA+KKF+ESEDOPV+KKIAL |
| | | | | | REIRMLKQLKHPNLVNL+EVFRR |
| | | | | | KR++HLVFEYCDHT+L+EL+R |
| | | | | | GV + ++KS+ WQTLQA+NFCH |
| | | | | | HNCIHRD+KPENILITK |
| | | | | | +IK+CDFGFA++L+ P D |
| | | | | | YTDYVATRWYR+PELLVGDTQY |
| | | | | | G VD+WAIGCVFAELL+G |
| | | | | | PLWPGKSDVDQLYLI +TLG |
| | | | | | LIPRHQ +F +N +F G+ |
| SGK038, ERK7 | 72 | P51954 | NEK1 (NIMA- | 17000030278391 | M+KY ++ IG+G+FGKA L K D |
| | | | RELATED | | +H VIKEIN K |
| | | | PROTEIN KINASE | | ENG L+IVM+YC+GGDL KRIN |
| | | | 1) [Mus musculus] | | Q+G LF EDQIL WFVQ$$ L |
| | | | | | LKH+HDRKILHRDIK+QNIFL+K+GV |
| | | | | | +LGDFGIARVLN+++ELARTCIGT |
| | | | | | PYYLSPEIC+NKPYNNK+DIW+LG |
| | | | | | CVLYELCTLKH FE N++ LVLKI |
| | | | | | F P+SP +S +L SL+SQLF+ |
| | | | | | +PRDRPS+NSIL+ |
| SGK158 | 100 | NP_064632.1 | Hypothetical | 11000283551497 | MLSIQDOAFINPHLAKIFERVRQS |
| | | | protein [Homo | | ADFMPLKQMMKTLNNDLGPNW |
| | | | sapiens] | | RDKLEYFEERPFAAASIGQVHLA |
| | | | | | RMKGGREVAMKIQYPGVAQSIN |
| | | | | | SDVNNLMAVLNMSNMLPEGLFP |
| | | | | | EHLIDVLRRELALECDYQREAAC |
| | | | | | ARKFRDLLKGHPFFYVPEIVDEL |
| | | | | | CSPHVLTTELVSGFPLDQAEGLS |
| | | | | | QEIRNEICYNILVLCLRELFEFHF |
| | | | | | MQTDPNWSNFFYDPQQHKVALL |
| | | | | | DFGATREYDRSFTDLYIQIIRAAA |
| | | | | | DRDR |
| SGK429 | 100 | AAD43192.1 | Putative human | 5001549_3 | IFVDNFVHADLHPGNILVQGANG |
| | | | protein [Homo | | LSSSQEAQLQQADICDTLVVAVP |
| | | | sapiens] | | SSLCPLRLVLLOAGIVAELQAPDL |
| | | | | | RNFRAVFMAVVMGQGQRVAELI |
| | | | | | LHHARASECRDVEGFKTEMAML |
| | | | | | VTQARKNTITLEKLHVSSLLSSVF |
| | | | | | KLLMTHKVKLESNFASIVFAIMVL |
| | | | | | EGLGRSLDPKLDILEAARPFLLTG |
| | | | | | PVCPP |
| SGK152, SUDD | 100 | NP_003822.1 | sudD (suppressor | 11000258249295 | MDLVGVASPEPGTAAAWGPSKC |
| | | | of bimD6, | | PWAIPQNTISCSLADVMSEQLAK |
| | | | Aspergillus | | ELQLEEEAAVFPEVAVAEGPFIT |
| | | | nidulans) homolog | | GENIDTSSDLMLAQMLQMEYDR |
| | | | [Homo sapiens] | | EYDAQLRREEKKFNGDSKVSISF |
| | | | | | ENYRKVHPYEDSDSSEDEVDWQ |
| | | | | | DTRDDPYRPAKPVPTPKKGFIGK |
| | | | | | GKDITTKHDEVVCGRKNTARME |
| | | | | | NFAPEFQVGDGIGMDLKLSNHVF |
| | | | | | NALKQHAYSEERRSARLHEKKE |
| | | | | | HSTAEKAVDPKTRLLMYKMVNS |
| | | | | | GMLETITG |
| SGK077 | 73 | BAB00640.1 | Hapsin [Mus | 11000283672012 | MA + P G+RLFRTY A R QR+ G |
| | | | musculus] | | A QWF P + +R F+SS S + S + |
| | | | | | DDPDDPDFPGS V +RRR P G |
| | | | | | +++ +LT TP+ + LR |
| | | | | | RPPQKCSTPC LR PPFP+ G L |
| | | | | | D SVC Q RD +ELG SASLFSS AS |
| | | | | | PG+P P L A SAVP HLP |
| | | | | | SL S+PC Q ATG R + + AS |
| | | | | | RS LF L+NSG |
| SGK093, Wnk3 | 73 | AAF31483.1 | Kinase deficient | 11000284063441 | PP +A PG P+ P T + ++ |
| | | | protein KDP [Homo | | ++ E P + G G E ER ++Q++ |
| | | | sapiens] | | E++ET+AV S |
| | | | | | DGR+LKFDIEIGRGSFKTVY+GLD |
| | | | | | T+TTVEVAWCELQ |
| | | | | | RKL+++ERQRF EE |
| | | | | | EMLKGLQHPNIVRFYDSW+S |
| | | | | | ++G+ |
| | | | | | CIVLVTELMTSGTLKTYL+RF+ |
| | | | | | MK +VL+ W RQIL+GL FLH+R |
| | | | | | PPI+HRDLKCDN+FITGPTGSVKI |
| | | | | | GDLGLATLKRASFAKSVIGTP |
| SGK074 | 67 | AAD41593.1 | Myak-S [Mus | 11000283476699 | ST+ Y+++E LG+GTFG+VAK |
| | | | musculus] | | W+RST E+VAIKILKN R + E+ |
| | | | | | +L + + +E + +R E F |
| | | | | | LVFE+LEQNL++F K+N F+PLP |
| | | | | | ++IR + QV TAL +LK L |
| | | | | | +IHADLKPENIMLVD R |
| | | | | | P+RVKVIDFGSAS S+ V |
| | | | | | Y+QSR+YRAPEI+LGLPFCE |
| | | | | | +D+WSLGCV+AEL LGWPLYPG |
| | | | | | +EYDQ+RYI +TQGLP +LL A K |
| | | | | | FF R+P+ |
| SGK087 | 100 | AAF91393.1 | DYRK4 [Homo | 17000036171996 | ENQAHNQMPASELKASEIPFHPS |
| | | | sapiens] | | IKTQDPKAEEKSPKKQKVTLTAA |
| | | | | | EALKLFKNQLSPYEQSEILGYAEL |
| | | | | | WFLGLEAKKLDTAPEKFSKTSFD |
| | | | | | DEHGFYLKVLHDHIAYRYEVLETI |
| | | | | | GKGSFGQVAKCLDHKNNELVAL |
| | | | | | KIIRNKKRFHQQALMELKILEALR |
| | | | | | KKDKDNTYNVVHMKDFFYFRNH |
| | | | | | FCITFELLGINLYELMKNNNFQGF |
| | | | | | SLSIVRRFTLSVLKCLQMLSVEKII |
| | | | | | HCDLKPENIVLYQKGQASVK |
| SGK295, KIS | 100 | NP_058989.1 | Kinase interacting | 17000113079883 | MAGSGCAWGAEPPRFLEAFGRL |
| | | | with stathmin | | WQVQSRLGSGSSASVYRVRCCG |
| | | | [Rattus norvegicus] | | PGSPPGALKQFLPPGTTGAAAS |
| | | | | | AAEYGFRKERAALEOLOGHRNIV |
| | | | | | TLYGVFTIHFSPNVPSRCLLLELL |
| | | | | | DVSVSELL+YSSHQGCSMWMIQ |
| | | | | | HCARDVLEALAFLHHEGYVHADL |
| | | | | | KPRNILWSAENECFKLIDFGLSFK |
| | | | | | EGNQDVKYIQTDGYRAPEAELQ |
| | | | | | NCLAOAGLQSDTECTSAVDLWS |
| | | | | | LGILLEMFSGMKLKHTVRSOEW |
| | | | | | KANSS |
| SGK419 | 100 | CAB70863.1 | Hypothetical | 5932598_100001_1 | MKKFSRMPKSEGGSGGGAAGG |
| | | | protein [Homo | | GAGGAGAGAGCGSGGSSVGVR |
| | | | sapiens] | | VFAVGRHQVTLEESLAEGGFSTV |
| | | | | | FLVRTHGGIRCALKRMYVNNMP |
| | | | | | DLNVCKREITIMKELSGHKNIVGY |
| | | | | | LDCAVNSISDNVWEVLILMEYCR |
| | | | | | AGQVVNQMNKKLQTGFTEPEVL |
| | | | | | QIFCDTCEAVARLHQCKTPIIHRD |
| | | | | | LKVENILLNDGGNYVLCDFGSAT |
| | | | | | NKFLNPQKDGVNVVEEEIKKYTT |
| | | | | | LSYRAPEMINLYGGKPITTKADIW |
| | | | | | ALGCL |
| SGK125, MYO3A | 100 | NP_059129.1 | Myosin IIIA [Homo | 17000078090910 | MFPLIGKTIIFDNFPDPSDTWEIT |
| | | | sapiens] | | TIGKGTYGKVFKVLNKKGQKAA |
| | | | | | VKILDPIHDIDEEIEAEYNILKALSD |
| | | | | | HPNVVRFYGIYFKKDKVNGDKL |
| | | | | | WLVLELCSGGSVTDLVKGFLKR |
| | | | | | GERMSEPLIAYILHEALMGLQHL |
| | | | | | HNNKTIHRDVKGNNILLTTEGGV |
| | | | | | KLVDFGVSAQLTSTRHRRNTSV |
| | | | | | GTPFWMAPEVIACEQQLDTTYD |
| | | | | | ARCDTWSLGITAIELGDGDPPLA |
| | | | | | DLHPMRALFKIPRNPPPKLRQPEL |
| SGK445 | 81 | AAC37649.1 | STK [Mus | 4000001803382 | FKVGNL GKGSFAGV + |
| | | | musculus] | | ESIHTGLE++++MI KKA+ |
| | | | | | +QRV NE+K+HCQLKHP LELY |
| | | | | | YF+D + |
| SGK127 | 61 | NP_038599.1 | Kinase suppressor | 17000062804843 | ++ +ALQQC +Q +ID+SI +L |
| | | | of ras [Mus | | GLRTKC+ |
| | | | musculus] | | SNDLTQ+EIRTLE+KLVKY +Q K |
| | | | | | V +R AEL+ +P+ W I +VR |
| | | | | | EV++EI P +L+L+ LLEM + + E + |
| | | | | | ++GA+ EEC+RL +L+CLR V GG |
| | | | | | + W +T+ ++ + PP S + |
| | | | | | P ++VAP PP S |
| | | | | | S P TP + + |
| SGK009, ANKRD3 | 100 | NP_065690.1 | Ankyrin repeat | 11000258161587 | MEGDGGTPWALALLRTFDAGEF |
| | | | domain 3 [Homo | | TGWEKVGSGGFGQVYKVRHVH |
| | | | sapiens] | | WKTWLAIKCSPSLHVDDRERME |
| | | | | | LLEEAKKMEMAKFRYILPVYGICR |
| | | | | | EPVGLVMEYMETGSLEKLLASEP |
| | | | | | LPWDLRFRIIHETAVGMNFLHCM |
| | | | | | APPLLHLDLKPANILLDAHYHVKIS |
| | | | | | DFGLAKCNGLSHSHDLSMDGLF |
| | | | | | GTIAYLPPERIREKSRLFDTKHDV |
| | | | | | YSFAIVIWGVLTQKKPFADEKNIL |
| | | | | | HIMVKVVKGHRPELPPVCRARPR |
| | | | | | AC |
| SGK421, STK22A, | 90 | NP_033461.1 | STK 22A | 6759077_1_4 | MDDAAVLKRRGY++GINLGEGSY |
| TSK1 | | | (spermiogenesis | | AKVKSAYSERLKFNVA+KIIDRKK |
| | | | associated) [Mus | | AP+DFLEKFLPREIEILAMLNH |
| | | | musculus] | | SI+KTYEIF TS GKVYIVMEL |
| | | | | | VQGDLLE IKTRGAL |
| | | | | | ED+ARKKFHQLS |
| | | | | | AIKYCHDLDVVHRDLK |
| | | | | | +NLLLDKDFNIKLSDF |
| | | | | | FSKRCLRDDSGR+ LSKTFCGS |
| | | | | | AYAAPEVLQGIPYQPKVYDIWSL |
| | | | | | GVILYIMVCGSMPYDDSNIKK |
| | | | | | LRIQKEHRVNFPRSKHLTGECKD |
| | | | | | LIY MLQP |
| SGK047 | 67 | NP_004320.1 | Bone | 11000258262374 | R + ++S CW PA+RL AL+I |
| | | | morphogenetic | | |
| | | | protein receptor, | | |
| | | | type IA [Homo | | |
| | | | sapiens] | | |
| SGK196 | 100 | BAB15623.1 | Unnamed protein | 17000062537825 | MEMQPQNSRRGLAPREVPPAVG |
| | | | product [Homo | | LLLIMALMNTLLYLCLDHFFIAPR |
| | | | sapiens] | | STVDPTHCPYGHFRIGQMKNCS |
| | | | | | PWLSCEELRTEVRQLKRVGEGA |
| | | | | | VKRVFLSEWKEHKVALSQLTSLE |
| | | | | | MKDDFLHGLQMLKSLQGTHVVT |
| | | | | | LLGYCEDDNTMLTEYHPLGSLSN |
| | | | | | LEETLNLSKYQNVNTWQHRLELA |
| | | | | | MDYVSIINYLHHSPVGTRVMCDS |
| | | | | | NDLPKTLSQYLLTSNFSILANDLD |
| | | | | | ALPLVNHSSGMLVKCGHRELHG |
| | | | | | DFVA |
| SGK396 | 50 | BAB11570.1 | Receptor-like | 5630059_1_4 | NA + ++PLN E L+ M GVA+GL |
| | | | protein kinase | | +H ++ HG++ +NVF + G + + |
| | | | [Arabidopsis | | + + L +PE+ + ++P |
| | | | thaliana] | | SD+Y++GL+L + K+GI V |
| SGK279, PKN | 95 | BAA36362.1 | Serin/threonine | 17000097259742 | MSVGCPEPEP SL CCGPG AP |
| | | | protein kinase | | PGAGVPLLTEDMQALTLRTLAAS |
| | | | [Rattus norvegicus] | | DVTKHYELVRELGKGTYGKVDLV |
| | | | | | YKGTGTKMALKFVNKSKTKLKNF |
| | | | | | LREVSITNSLSSSPFIIKVFOVVFE |
| | | | | | TE+CYVFAQEYAPAGDLFOIIPPQ |
| | | | | | VGLPEDTVKRCVQQLGLALDFMH |
| | | | | | RQLVHRDIKPENVLLFDRECRRV |
| | | | | | KLADFGMTRRVGCRVKRVSGTI |
| | | | | | YTAPEVCQAGRADG |
| | | | | | AVDTGVDVWAFGVLIFCVLTGNF |
| | | | | | PWEA |
| SGK037 | 83 | P51954 | NEK1 [Mus | 17000030265658 | M+KY ++ IG+G+FGKA L K D |
| | | | musculus] | | +H VIKEIN +M +E++ S++EV +L |
| | | | | | MKHPNIV + SF+ENG |
| | | | | | L+IVM+YC+GGDL KRIN Q+G LF |
| | | | | | EDQIL WFVQI L |
| | | | | | LKH+HDRKILHRDIK+QNIFL+K+GV |
| | | | | | +LGDFGIARVLN+++ELARTCIGT |
| | | | | | PYYLSPEIC+NKPYNNK+DIW+LG |
| | | | | | CVLYELCTLKH FE N++ LVLKI |
| | | | | | F P+SP +S +L SL+SQLF+ |
| | | | | | +PRORPS+NSIL++ F+ I |
| | | | | | K+L+P++ E |
| SGK060 | 100 | BAB15672.1 | Unnamed protein | 17000036897142 | MGSCDLATTLTGTPHYMSPEAL |
| | | | product [Homo | | HQGYDTKSDIWSLACILYEMCCM |
| | | | sapiens] | | NHAFAGSNFLSIVLKIVEGDTPSL |
| | | | | | PERYPKELNAIMESMLNKNPSLR |
| | | | | | PSAIEILKIPYLDEQLQNLMCRYS |
| | | | | | EMTLEDKNLDCQKEAAHIINAMQ |
| | | | | | KRIHLQTLRALSEVQKMTPRERM |
| | | | | | RLRKLQAADEKARKLKKIVEEKY |
| | | | | | EENSKRMQELRSRNFQQLSVDV |
| | | | | | LHEKTHLKGMEEKEEQPEGRLS |
| | | | | | CSPQDEDEERWQGREEESDEP |
| | | | | | TLEN |
| SGK080 | 93 | NP_002488.1 | NIMA (never in | 17000048119006 | MPSRAE+YEVLYTIGTGS |
| | | | mitosis gene a)- | | GRCQKI+RKSDGKILVWKEL |
| | | | related kinase | | YGSMTEAEKQMLVSEVNLL |
| | | | [Homo sapiens] | | ELK+PNIV |
| | | | | | YYDRIIDRTNTTLYIVMEYCE |
| | | | | | GDLASVITKGTKERQYLDEEFVL |
| | | | | | RV TQLTLALK CHRRSDG HTV+ |
| | | | | | RDLKPA+VFLDGKQNVKLGD |
| | | | | | GLARILNHDTSFAKTFVGTPYYM |
| | | | | | SPEQ N MSYNEK DIWSLGCL YE |
| | | | | | ALMPPFTAFSQKELAGKIREGKF |
| | | | | | RRI YRYSDELNEII RM LK |
| SGK002 | 94 | P36507 | MEK2 [Homo | 11000257702671 | MLARRKP+LPALYINPTIAEGPSP |
| | | | sapiens] | | TSEGASEANLVDLQKKLEELELD |
| | | | | | EQQ |
| | | | | | KRLEAFLTQKAKVGELKDODFER |
| | | | | | SEL |
| | | | | | AGNGGVVTKVQHRPSGLIMARK |
| | | | | | LIHLEIKPAIRNQIIRE |
| | | | | | QVLHECNSPYIVGFYGAFY D |
| | | | | | EISICMEHMDGGSLDQ |
| | | | | | LKEAKRIPE+ILGKVSIAVLRGLAY |
| | | | | | LREKHQ1MHR+VKPSNILVNSRG |
| | | | | | EIKLCDFGVSGQLIDSMANSFVG |
| | | | | | TRSYMAPERLQGTHYSVQS |
| | | | | | IWSM L |
| SGK058 | 99 | BAB15538.1 | Unnamed protein | 17000036877196 | MEFVPGGSISSIINRFGPLPEMVF |
| | | | product [Homo | | CKYTKQ1LQGVAYLHENCVVHRD |
| | | | sapiens] | | IKGNNVMLMPTGIIKLIDFGCARR |
| | | | | | LAWAGLNGTHSDMLKSMHGTPY |
| | | | | | WM |
| | | | | | PEVINESGYGRKSDIWSIGCTVF |
| | | | | | EMATGKPPLASMDRMAAMFYIG |
| | | | | | AHRGLMPPLPDHFSENAADFVR |
| | | | | | MCLTR |
| SGK103 | 68 | CAA39285.1 | Fused [Drosophila | 11000284272557 | OL PQN+LLDK++ L FGLAR L |
| | | | melanogaster] | | |
| SGK035 | 95 | Q13177 | PAK-2 [Homo | 17000030169905 | PPVIAP PDH KSIYTR+VIDPV |
| | | | sapiens] | | APVGDS+VDG AKSLDKQKKK |
| | | | | | KMTDEEIMEKLRTIVSIG |
| | | | | | +KKYTRYEKIGQGASGTVFTATD |
| | | | | | VALGQ+VAIKQINLQKQPKKELIIN |
| | | | | | EILVMKELKNPNIVNFLDSYLVGD |
| | | | | | ELFVV+EYLA |
| | | | | | GSLTDVVTETCMDEAQ1AAVCRE |
| | | | | | LOALEFLHANQVIHRDIKSD+VLL |
| | | | | | GMEGSVKLTDFGFCAQITPEQS |
| | | | | | RST+V |
| | | | | | TP+WMAPEVVTRKAYGPKV |
| | | | | | +WSLGIMA |
| SGK075 | 80 | NP_059129.1 | Myosin IIIA [Homo | 11000283492249 | ++ PDP+DTWEI |
| | | | sapiens] | | ETIGKGTYGKV+KV NK++G |
| | | | | | AAVKILDP+ D+DEEIEAEYNIL+ L |
| | | | | | +HPNVV+FYG+++KD G |
| | | | | | +LWLVLELC+GGSVT+LVKG L+ |
| | | | | | G+R+ E +I+YIL+ |
| | | | | | AL+GLQHLHNN+ |
| | | | | | IHRDVKGNNILLTTEGGVKLVDF |
| | | | | | VSAQLTSTR |
| | | | | | RRNTSVGTPFWMAPEVIACEQQ |
| | | | | | D++YDARCD |
| | | | | | WSLGITAIELGDGDPPL D+HP++ |
| | | | | | LFKIPRNPPP L PE W EFN FIS+ |
| SGK188, EphA9 | 71 | Q61772 | Ephrin Type-A | 17000057739161 | LC LLG G A+EV+LLDSKA Q |
| | | | receptor 7 [Mus | | EL W + P +GWEEISG+DE+ |
| | | | musculus] | | PIRTYQVC V+EPNQ+NWL+T |
| | | | | | WIS+G |
| | | | | | ORIFVEL+FTLRDC+S+PG |
| | | | | | GTCKETFN+YY ET+ D GR + + |
| | | | | | KIDTIAADESFTOGDLGERKMKL |
| | | | | | NTEVREIGPLS++GF+LAFQDVG |
| | | | | | AC+ALVSV+VYYK+C V LA FP |
| | | | | | T S FS+LVEV GTCV+ +E E + |
| | | | | | PRMHC A+GEWLVP+G+C |
| SGK040 | 52 | P41243 | BATK [Rattus | 11000257912897 | GA G+G FG+V + Y G++VAVK |
| | | | norvegicus] | | ++ E V+ L H +L+ LL + + |
| | | | | | +VME SKG+L L+ +L TQ + |
| | | | | | ALHVA+G+ YL S +++RDL N+L+ |
| | | | | | + ++AK++D+G+A+ R G+ +S |
| | | | | | + APE + N ++ |
| | | | | | ++DV+SFG+LL+++ + G + + |
| | | | | | +E ++ P P P V L+ C+ P |
| | | | | | RP ++ + L |
| SGK390 | 94 | Q64398 | Diacylglycerol | 17000112562523 | MAGAG QHHPP GGAA G A |
| | | | kinase eta | | +A AGPGEDSSDSEAEQ |
| | | | [Mesocricetus | | GPQKLIRKVSTSGQIRTKTSIKEG |
| | | | auratus] | | QLLKQTSSFQRWKKRYFKLRGR |
| | | | | | TLYYAKDSKSLIFDEVDLSDASVA |
| | | | | | EASTKNANNSFTIITPFRRLMLCA |
| | | | | | ENRKEMEDWISSLKSVQ+REPY |
| | | | | | EVAQFNVEHFSGMHNWYACSH |
| | | | | | ARPTFCNVCRESLSGVTSHGLS |
| | | | | | CEVCKFKAHKRCAVRATNNCKW |
| | | | | | TTLASIGKDIIEDEDGVAMPHQW |
| | | | | | LEGNL |
| SGK007 | 62 | T42260 | Guanylate cyclase | 11000257855312 | MA + E P+E G E HA H +LVL |
| | | | [Rattus norvegicus] | | LF +L+TCLEAAKLTVGF |
| | | | | | PWNISHPFS+QRLGAGLQ1A+DK |
| | | | | | +NSE V GN + TYTN+ C+ |
| | | | | | KESLA F +QVQ E IS L |
| | | | | | GPACPEAAE |
| | | | | | +IG VL+ESLQYLGW++IG+FGG S |
| | | | | | SSW V+ELW+ VE+EL+ HF ITA |
| | | | | | +RY++ LLOE L +SS+ARVIIL |
| | | | | | CSSEDA |
| SGK050 | 71 | NP_003986.2 | Natriuretic peptide | 17000035767558 | R++ ++ RF G CI P++ IV |
| | | | receptor B | | EYCP+GSL D+L ND+I+ +W F |
| | | | precursor, isoform | | |
| | | | b [Homo sapiens] | | |

Table 5 describes domains in the proteins outside of the kinase catalytic domain. The column headings are: “Gene Name”, “ID#na” (nucleotide 1-255 of . . . ), “ID#aa” (residues 1-255 of . . . ), “Extracatalytic Domains (AA boundaries)”. Extracatalytic domains were identified by performing hidden Markov searches of the amino acid sequences using Pfam, a large collection of multiple sequence alignments and hidden Markov models covering many common protein domains. Version 5.1 of Pfam (September 2000) contains alignments and models for 2015 protein families (http://pfam.wustl.edu/faq.shtml). The PFAM alignments were downloaded from http://pfam.wustl.edu/hmmsearch.shtml and the HMMr searches were run locally on a Timelogic computer (TimeLogic Corporation, Incline Village, Nev.). The PFAM accession number, the length in amino acids and the number of proteins used to build the profile are listed below.

The CNH domain (Pfam PF00780) is approximately 300 amino acids long. It is built from 23 members and found in NIK 1-like kinase, mouse citron and yeast ROMI and ROM2. The PKC terminal domain (PF00433) is approximately 66 amino acids long. It is built from 235 members and found in protein kinase C from multiple species. The phorbol esters/diacylglycerol-binding domain (C1 domain) (PF00130) is approximately 50 amino acids long. It is built from 269 members and in found in protein kinase C from multiple species. The RGS regulator of G protein signaling domain (PF00615) is approximately 125 amino acids long. It is built from 103 members and found in RGS (Regulator of G protein Signaling) family members that include the GTPase-activating proteins for heterotrimeric G-protein alpha-subunits The PDZ domain (PF00595) is approximately 83 amino acids long. It is built from 721 members and found in membrane-associated proteins that include homologues of the MAGUK family of guanylate kinases, several protein phosphatases and protein kinases. PDZ domains are also found in neuronal nitric oxide synthase as well as in the subfamily of dystrophin-associated proteins, collectively known as syntrophins. The Octicosapeptide domain (PF00564) is approximately 30 amino acids long. It is built from 47 members that include NADPH oxidase subunits, sorting nexins and PtdIns 3-kinases. This motif may be involved in Ca++ binding. The cyclin domain (PF00134) is approximately 267 amino acids long. It is built from 233 members that include cyclins, TFIIB and RB/p 107. The RNA recognition domain (also known as RRM, RBD, or RNP) (PF00076) is approximately 71 amino acids long. It is built from 1335 members that include a variety of RNA-binding proteins such hnRNP proteins, proteins implicated in regulation of alternative splicing, and protein components. The motif is also found in a few single-stranded DNA-binding proteins. The myosin head domain (PF00063) is approximately 409 amino acids long. It is built from 310 members that include the motor proteins such as myosin. The ankyrin domain (PF00023) is approximately 33 amino acids long. It is built from 2220 members that include the ankyrin family of structural proteins, CDK inhibitors such as p19INK4d, and other signaling proteins such as the nuclear factor NF-kappa-b p50 subunit and Bc13 (b-cell lymphoma 3-encoded protein) among others. The ankyrin repeats generally consist of a beta, alpha, alpha, beta order of secondary structures. The repeats associate to form a higher order structure. The ephrin Receptor ligand binding domain (PF01404) is approximately 171 amino acids long. It is built from 52 members that include the Eph family of receptor tyrosine kinases. The fibronectin type III domain (PF00041) is approximately 85 amino acids long. It is built from 2468 members that include a variety of transmembrane and membrane-associated proteins that include fibronectin, cytokine receptors, receptor tyrosine kinases, receptor tyrosine phosphatases, etc. The SAM domain (Sterile alpha motif) (PF00536) is approximately 110 amino acids long. It is built from 64 members. The SAM domain is an evolutionarily conserved protein-binding region that is involved in the regulation of numerous developmental processes in diverse eukaryotes. The SAM domain can potentially function as a protein interaction module through its ability to homo- and hetero oligomerise with other SAM domains. The DAG Diacylglycerol (DAG) domain (PF00609 ) is approximately 166 amino acids long. It is built from 27 members from the diacylglycerol kinase subfamily of protein kinases. This domain is assumed to be an accessory domain in diacylglycerol binding. The ROI 1 domain (PF01163) is approximately 570 amino acids in length and is known generally in the art (see, e.g. www.pfam.wustl.edu). It is built from 14 members and is believed to encode an atypical A-Kinase.

TABLE 5
Extracatalytic Domains
413406_1.xls

| Gene | | | Extracatalytic | | | | |
|---|---|---|---|---|---|---|---|
| Name | ID#na | ID#aa | Domains (AA boundaries) | Genomic_ID | Comparison | Cel Sort | |
| SGK187, | 1 | 58 | CNH domain (1620-1917); | 4926912_1_1 | | 44 | |
| CRIK | | | PH 1472-1591; | | | | |
| | | | Protein kinase C | | | | |
| | | | terminal domain (362-391); | | | | |
| | | | Phorbol esters/ | | | | |
| | | | diacylglycerol | | | | |
| | | | binding domain | | | | |
| | | | (C1 domain) (1391-1439) | | | | |
| SGK064, | 2 | 59 | RGS | 11000284009826 | | 23 | 4_SEQ_ID_1_11000284009826 |
| GRK7 | | | (55-176) | | | | |
| SGK409, | 3 | 60 | PDZ domain | 6671897_1_1 | | 54 | |
| KIAA0303 | | | (1020-1148) | | | | |
| SGK410 | 5 | 62 | Protein kinase C | 7018620_2 | | 55 | |
| | | | terminal domain (558-624); | | | | |
| | | | Phorbol esters/ | | | | |
| | | | diacylglycerol binding | | | | |
| | | | domain (C1 domain) | | | | |
| | | | (176-225); | | | | |
| | | | Octicosapeptide | | | | |
| | | | repeat (100-129) | | | | |
| SGK093, | 31 | 88 | Cyclin (1373-1410) | 11000284063441 | | 33 | 4_SEQ_ID_38_11000284063441 |
| Wnk3 | | | | | | | |
| SGK295, | 34 | 91 | RNA recognition | 17000113079883 | | 49 | |
| KIS | | | motif (345-401) | | | | |
| SGK125, | 36 | 93 | Myosin head | 17000078090910 | | 38 | |
| MYO3A | | | (340-1040); IQ, | | | | |
| | | | (3 domains, 1055-1366) | | | | |
| SGK127 | 38 | 95 | Phorbol esters/ | 17000062804843 | | 39 | |
| | | | diacylglycerol binding | | | | |
| | | | domain (C1 domain) | | | | |
| | | | (408-451) | | | | |
| SGK009, | 39 | 96 | Ankyrin (10 domains: | 11000258161587 | P | 6 | 1_SEQ_ID_37_11000258161587 |
| ANKRD3 | | | 437-469, 470-502, | | +DLGTTDVQKK | | |
| | | | 503-535, 536-568, | | KLVDAIVSGDTS | | |
| | | | 569-602, | | | | |
| | | | 602-635, 636-668, | | | | |
| | | | 669-701, | | | | |
| | | | 702-730, 738-770) | | | | |
| SGK188, | 53 | 110 | Ephrin receptor | 17000057739161 | C+ G P +EV | 45 | |
| EphA9 | | | ligand binding domain | | LLDSK Q | | |
| | | | (35-211); Fibronectin | | ELGW + | | |
| | | | type III domain (2 domains: | | PS+GWEEISGV | | |
| | | | 339-436 & 454-537); | | DEH | | |
| | | | SAM domain (Sterile | | PIRTYQVCNV++ | | |
| | | | alpha motif)(931-996) | | +Q+NWL+T W+R | | |
| | | | | | Q+I+VEL+FTLR | | |
| | | | | | DC+SIP | | |
| | | | | | GTCKETFN+YY | | |
| | | | | | +E++ + R + | | |
| | | | | | KIDTIAADESFT | | |
| | | | | | Q DLG+R | | |
| | | | | | +KLNTE+RE+G | | |
| | | | | | P++++GF+LAFQ | | |
| | | | | | DVGACVALVSV | | |
| | | | | | RVY+K+C TV+ | | |
| | | | | | LA FP T + | | |
| | | | | | +LVEV G+CV | | |
| | | | | | +S+ E | | |
| | | | | | PPRM+C | | |
| | | | | | +GEWLVP+G+C | | |
| | | | | | +C+AG | | |
| SGK390 | 55 | 112 | Diacylglycerol kinase | 17000112562523 | | 52 | |
| | | | catalytic domain (332-457); | | | | |
| | | | DAG_PE-bind (176-225 & | | | | |
| | | | 248-298); PH | | | | |
| | | | domain (68-158) | | | | |
| | | | Diacylglycerol kinase | | | | |
| | | | accessory domain | | | | |
| | | | (770-927); | | | | |

Table 6 shows the results of a gene expression analysis of the kinases presented in this application using a microarray of cDNAs derived from 469 tissues and cell lines. The cDNAs were spotted on nylon and probed with labeled kinase genes, as described in Materials and Methods below. The kinase probes were PCR cloned from genomic exons. Data presentation from left to right is as follows: “Tissue”: tissue type of the cDNA; “Tumor sym”, indicates that the tissue is derived from a tumor, “sym” refers to the fact that the 5′ and 3′ primers used to make the sample are the same; “Normal Sym”, indicates normal tissue was used to make the sample, with symmetric primers as described above; “Tumor 1o”, indicates that primary tumor tissue was used to make the cDNA; “Tumor cells”, indicates that these cDNA samples were made from cultured tumor cells; “Normal”, indicates that these samples are derived from normal tissue or cell lines; “Endos”, indicates that these samples are derived from endothelium-related tissue sources; “p53” refers to the status, mutant or wild-type, of the p53 gene in the source samples. Normalized expression values are presented for each gene referred to by its SEQ_ID# on the subsequent columns. Genes represented in Table 6 are: SGK187 (SEQ ID NO: 1); SGK124 (SEQ ID NO: 9); SGK386 (SEQ ID NO: 21); SGK003 (SEQ ID NO: 22); SGK093 (SEQ ID NO: 31); SGK074 (SEQ ID NO: 32); and SGK396 (SEQ ID NO: 43).

TABLE 6
Tissue Array
413406_1.xls

| | Tumor | Normal | Tumor | Tumor | | | | SGK187 | SGK124 | SGK386 | SGK003 | SGK093 | SGK074 | SGK396 | | | | NCI/ |
|---|---|---|---|---|---|---|---|---|---|---|---|---|---|---|---|---|---|---|
| Tissue | sym | sym | 1o | cells | Normal | Endos | p53 | ID#NA_1 | ID#NA_9 | ID#NA_21 | ID#NA_22 | ID#NA_31 | ID#NA_32 | ID#NA_43 | Release | tag | pos | etc |
| TCGP | 26 | | | | | | | 212 | 0 | 0 | 2,366 | 0 | 58,451 | 2,077 | R1 384-1 | sym | B02 | 284 |
| n keratinocytes | 46 | | | | | | | 83 | 3,050 | 0 | 2,340 | 0 | 89,541 | 1,223 | R1 384-1 | sym | B22 | 330 |
| Feb. 25, 1992 #10 | | | | | | | | | | | | | | | | | | |
| h adult SMC | 47 | | | | | | | 0 | 0 | 0 | 1,713 | 374 | 73,933 | 291 | R1 384-1 | sym | B23 | 283 |
| Oct. 21, 1992 #17 | | | | | | | | | | | | | | | | | | |
| h fibroblasts | 48 | | | | | | | 0 | 0 | 0 | 423 | 152 | 62,710 | 1,235 | R1 384-1 | sym | B24 | 329 |
| Mar. 31, 1992 #12 | | | | | | | | | | | | | | | | | | |
| HOP-92 | 97 | | | | | | | 514 | 0 | 0 | 2,631 | 94 | 87,584 | 1,316 | R1 384-1 | sym | E01 | 224 |
| OVCAR-8 | 98 | | | | | | | 16 | 0 | 0 | 0 | 0 | 4,204 | 164 | R1 384-1 | sym | E02 | 236 |
| EKVX | 99 | | | | | | | 19 | 0 | 0 | 231 | 0 | 27,721 | 105 | R1 384-1 | sym | E03 | 225 |
| IGROV1 | 100 | | | | | | | 0 | 0 | 0 | 65 | 0 | 7,800 | 0 | R1 384-1 | sym | E04 | 237 |
| NCI-H23 | 101 | | | | | | | 0 | 0 | 0 | 0 | 0 | 26,620 | 70 | R1 384-1 | sym | E05 | 226 |
| SK-OV-3 | 102 | | | | | | | 260 | 0 | 0 | 1,474 | 0 | 65,008 | 1,516 | R1 384-1 | sym | E06 | 238 |
| NCI-H226 | 103 | | | | | | | 153 | 365 | 0 | 1,091 | 91 | 107,932 | 3,834 | R1 384-1 | sym | E07 | 227 |
| SNB-19 | 104 | | | | | | | 0 | 0 | 0 | 855 | 0 | 74,412 | 1,302 | R1 384-1 | sym | E08 | 239 |
| NCI-H322M | 105 | | | | | | | 243 | 77 | 0 | 422 | 226 | 35,551 | 84 | R1 384-1 | sym | E09 | 228 |
| SNB-75 | 106 | | | | | | | 138 | 0 | 0 | 0 | 0 | 98,053 | 1,797 | R1 384-1 | sym | E10 | 240 |
| NCI-H460 | 107 | | | | | | | 0 | 548 | 0 | 3,348 | 81 | 78,829 | 1,166 | R1 384-1 | sym | E11 | 229 |
| U251 | 108 | | | | | | | 0 | 554 | 0 | 0 | 0 | 84,310 | 677 | R1 384-1 | sym | E12 | 241 |
| NCI-H522 | 109 | | | | | | | 196 | 0 | 0 | 162 | 0 | 22,554 | 497 | R1 384-1 | sym | E13 | 230 |
| SF-268 | 110 | | | | | | | 0 | 771 | 0 | 0 | 593 | 49,287 | 1,115 | R1 384-1 | sym | E14 | 242 |
| A549/ATCC | 111 | | | | | | | 0 | 266 | 0 | 1,613 | 1,619 | 43,786 | 1,448 | R1 384-1 | sym | E15 | 231 |
| SF-295 | 112 | | | | | | | 0 | 0 | 0 | 1,333 | 0 | 7,106 | 0 | R1 384-1 | sym | E16 | 243 |
| HOP-62 | 113 | | | | | | | 231 | 343 | 0 | 529 | 1,746 | 21,968 | 670 | R1 384-1 | sym | E17 | 232 |
| SF-539 | 114 | | | | | | | 176 | 101 | 0 | 237 | 167 | 39,588 | 1,662 | R1 384-1 | sym | E18 | 244 |
| OVCAR-3 | 115 | | | | | | | 0 | 179 | 0 | 998 | 442 | 28,838 | 737 | R1 384-1 | sym | E19 | 233 |
| CCRF-CEM | 116 | | | | | | | 80 | 6,816 | 0 | 605 | 0 | 45,975 | 2,130 | R1 384-1 | sym | E20 | 245 |
| OVCAR-4 | 117 | | | | | | | 52 | 57 | 0 | 704 | 292 | 34,439 | 1,510 | R1 384-1 | sym | E21 | 234 |
| K-562 | 118 | | | | | | | 0 | 0 | 0 | 0 | 185 | 33,123 | 1,072 | R1 384-1 | sym | E22 | 246 |
| OVCAR-5 | 119 | | | | | | | 0 | 0 | 0 | 1,307 | 542 | 18,164 | 23 | R1 384-1 | sym | E23 | 235 |
| MOLT-4 | 120 | | | | | | | 0 | 0 | 0 | 118 | 133 | 35,133 | 410 | R1 384-1 | sym | E24 | 247 |
| HL-60 | 121 | | | | | | | 0 | 537 | 0 | 0 | 659 | 80,247 | 719 | R1 384-1 | sym | F01 | 248 |
| SN12C | 122 | | | | | | | 32 | 0 | 0 | 0 | 711 | 76,151 | 1,579 | R1 384-1 | sym | F02 | 260 |
| RPMI 8226 | 123 | | | | | | | 0 | 0 | 0 | 0 | 0 | 15,177 | 419 | R1 384-1 | sym | F03 | 249 |
| A498 | 124 | | | | | | | 0 | 904 | 0 | 430 | 3,358 | 122,082 | 962 | R1 384-1 | sym | F04 | 261 |
| SR | 125 | | | | | | | 80 | 0 | 0 | 0 | 553 | 97,140 | 1,884 | R1 384-1 | sym | F05 | 250 |
| Caki-1 | 126 | | | | | | | 0 | 0 | 0 | 1,099 | 6,386 | 88,518 | 1,978 | R1 384-1 | sym | F06 | 262 |
| OU-145 | 127 | | | | | | | 560 | 191 | 0 | 169 | 0 | 3,687 | 0 | R1 384-1 | sym | F07 | 251 |
| RXF 393 | 128 | | | | | | | 51 | 202 | 0 | 685 | 1,192 | 108,097 | 1,825 | R1 384-1 | sym | F08 | 263 |
| PC-3 | 129 | | | | | | | 0 | 0 | 0 | 0 | 55 | 27,208 | 99 | R1 384-1 | sym | F09 | 252 |
| ACHN | 130 | | | | | | | 0 | 0 | 0 | 1,248 | 0 | 21,998 | 0 | R1 384-1 | sym | F10 | 264 |
| HCC-2998 | 131 | | | | | | | 0 | 0 | 0 | 0 | 0 | 14,182 | 0 | R1 384-1 | sym | F11 | 253 |
| 786-0 | 132 | | | | | | | 0 | 443 | 0 | 500 | 302 | 74,251 | 689 | R1 384-1 | sym | F12 | 265 |
| HCT 116 | 133 | | | | | | | 489 | 149 | 0 | 397 | 0 | 2,376 | 0 | R1 384-1 | sym | F13 | 254 |
| TK-10 | 134 | | | | | | | 94 | 102 | 0 | 1,682 | 415 | 51,674 | 1,842 | R1 384-1 | sym | F14 | 266 |
| SW-620 | 135 | | | | | | | 503 | 25 | 148 | 21,381 | 90 | 479,097 | 853 | R1 384-1 | sym | F15 | 255 |
| LOX IMVI | 136 | | | | | | | 865 | 0 | 0 | 0 | 610 | 8,504 | 241 | R1 384-1 | sym | F16 | 267 |
| COLO 205 | 137 | | | | | | | 608 | 348 | 0 | 223 | 628 | 60,452 | 610 | R1 384-1 | sym | F17 | 256 |
| Malme-3M | 138 | | | | | | | 134 | 260 | 0 | 416 | 734 | 33,878 | 1,065 | R1 384-1 | sym | F18 | 268 |
| HCT-15 | 139 | | | | | | | 69 | 281 | 0 | 0 | 781 | 35,146 | 562 | R1 384-1 | sym | F19 | 257 |
| SK-MEL-2 | 140 | | | | | | | 720 | 219 | 0 | 1,523 | 340 | 21,933 | 648 | R1 384-1 | sym | F20 | 269 |
| KM-12 | 141 | | | | | | | 0 | 227 | 0 | 0 | 0 | 23,168 | 0 | R1 384-1 | sym | F21 | 258 |
| SK-MEL-5 | 142 | | | | | | | 346 | 1,100 | 0 | 1,862 | 319 | 20,650 | 825 | R1 384-1 | sym | F22 | 270 |
| UO-31 | 143 | | | | | | | 1,235 | 0 | 0 | 0 | 0 | 43,858 | 549 | R1 384-1 | sym | F23 | 259 |
| SK-MEL-28 | 144 | | | | | | | 0 | 121 | 0 | 362 | 151 | 2,283 | 158 | R1 384-1 | sym | F24 | 271 |
| UACC-62 | 145 | | | | | | | 0 | 74 | 0 | 473 | 1,045 | 106,158 | 1,533 | R1 384-1 | sym | G01 | 272 |
| UACC-257 | 147 | | | | | | | 1,027 | 131 | 0 | 1,889 | 0 | 132,416 | 2,031 | R1 384-1 | sym | G03 | 273 |
| M14 | 149 | | | | | | | 0 | 182 | 0 | 67 | 0 | 14,280 | 59 | R1 384-1 | sym | G05 | 274 |
| MCF7 | 151 | | | | | | | 66 | 0 | 106 | 17,887 | 0 | 476,093 | 791 | R1 384-1 | sym | G07 | 275 |
| MCF-7/ADR-RES | 153 | | | | | | | 0 | 0 | 0 | 972 | 484 | 87,261 | 1,487 | R1 384-1 | sym | G09 | 276 |
| Hs 578T | 155 | | | | | | | 0 | 0 | 0 | 1,717 | 0 | 125,561 | 611 | R1 384-1 | sym | G11 | 277 |
| MDA-MB-231 | 157 | | | | | | | 0 | 416 | 0 | 0 | 107 | 77,227 | 0 | R1 384-1 | sym | G13 | 278 |
| MDA-MB-435 | 159 | | | | | | | 391 | 359 | 0 | 1,576 | 0 | 41,654 | 1,257 | R1 384-1 | sym | G15 | 279 |
| MDA-N | 161 | | | | | | | 0 | 183 | 0 | 2,141 | 380 | 28,371 | 1,457 | R1 384-1 | sym | G17 | 280 |
| T-47D | 163 | | | | | | | 139 | 103 | 0 | 0 | 202 | 52,411 | 220 | R1 384-1 | sym | G19 | 281 |
| adrenal gland-h | | 1 | | | | | | 64 | 29 | 0 | 16,403 | 133 | 808,208 | 1,624 | R1 384-1 | sym | A01 | 285 |
| lymph node-h | | 2 | | | | | | 0 | 858 | 0 | 2,522 | 0 | 126,297 | 6,190 | R1 384-1 | sym | A02 | 297 |
| bone marrow-h | | 3 | | | | | | 0 | 793 | 0 | 157 | 435 | 127,325 | 3,459 | R1 384-1 | sym | A03 | 286 |
| mammary gland-h | | 4 | | | | | | 0 | 0 | 0 | 0 | 221 | 60,842 | 482 | R1 384-1 | sym | A04 | 298 |
| brain-h | | 5 | | | | | | 0 | 309 | 0 | 206 | 884 | 109,916 | 4,188 | R1 384-1 | sym | A05 | 287 |
| pancreas-h | | 6 | | | | | | 0 | 400 | 0 | 1,126 | 0 | 103,318 | 2,814 | R1 384-1 | sym | A06 | 299 |
| cerebellum-h | | 7 | | | | | | 0 | 87 | 0 | 9,410 | 578 | 167,028 | 9,694 | R1 384-1 | sym | A07 | 288 |
| pituitary gland-h | | 8 | | | | | | 83 | 1,273 | 0 | 1,820 | 194 | 106,893 | 5,097 | R1 384-1 | sym | A08 | 300 |
| fetal brain-h | | 9 | | | | | | 359 | 1,004 | 0 | 4,928 | 862 | 121,604 | 8,651 | R1 384-1 | sym | A09 | 289 |
| placenta-h | | 10 | | | | | | 0 | 0 | 0 | 6,212 | 1,310 | 115,612 | 2,852 | R1 384-1 | sym | A10 | 301 |
| fetal kidney-h | | 11 | | | | | | 0 | 0 | 0 | 4,629 | 28,058 | 192,142 | 4,075 | R1 384-1 | sym | A11 | 290 |
| prostate, h | | 12 | | | | | | 0 | 290 | 0 | 851 | 0 | 18,517 | 0 | R1 384-1 | sym | A12 | 302 |
| fetal liver-h | | 13 | | | | | | 5,768 | 69 | 0 | 2,652 | 7,321 | 128,455 | 2,039 | R1 384-1 | sym | A13 | 291 |
| salivary gl.-h | | 14 | | | | | | 0 | 0 | 0 | 1,389 | 0 | 92,749 | 1,033 | R1 384-1 | sym | A14 | 303 |
| fetal lung-h | | 15 | | | | | | 0 | 666 | 0 | 3,837 | 1,462 | 198,037 | 4,592 | R1 384-1 | sym | A15 | 292 |
| skeletal muscle-h | | 16 | | | | | | 1,336 | 0 | 0 | 2,995 | 0 | 43,019 | 42 | R1 384-1 | sym | A16 | 304 |
| heart-h | | 17 | | | | | | 12,477 | 125 | 0 | 2,160 | 205 | 107,943 | 899 | R1 384-1 | sym | A17 | 293 |
| small intestine-h | | 18 | | | | | | 0 | 226 | 0 | 1,736 | 1,221 | 117,752 | 882 | R1 384-1 | sym | A18 | 305 |
| kidney-h | | 19 | | | | | | 0 | 56 | 0 | 2,418 | 18,839 | 135,359 | 1,267 | R1 384-1 | sym | A19 | 294 |
| spinal cord-h | | 20 | | | | | | 0 | 144 | 0 | 1,031 | 451 | 119,848 | 789 | R1 384-1 | sym | A20 | 306 |
| liver-h | | 21 | | | | | | 379 | 1,481 | 0 | 433 | 0 | 153,048 | 852 | R1 384-1 | sym | A21 | 295 |
| Spleen-h | | 22 | | | | | | 778 | 191 | 0 | 861 | 0 | 122,519 | 1,248 | R1 384-1 | sym | A22 | 307 |
| lung-h | | 23 | | | | | | 0 | 530 | 0 | 1,794 | 0 | 76,316 | 733 | R1 384-1 | sym | A23 | 296 |
| stomach-h | | 24 | | | | | | 372 | 0 | 0 | 171 | 0 | 39,162 | 278 | R1 384-1 | sym | A24 | 308 |
| testis-h | | 25 | | | | | | 15 | 0 | 0 | 14,003 | 0 | 141,552 | 5,293 | R1 384-1 | sym | B01 | 309 |
| thymus-h | | 27 | | | | | | 1,943 | 343 | 0 | 2,504 | 0 | 180,985 | 6,498 | R1 384-1 | sym | B03 | 310 |
| HPAEC | | 28 | | | | 28 | | 0 | 0 | 0 | 2,192 | 0 | 171,007 | 2,973 | R1 384-1 | sym | B04 | 319 |
| thyroid gland-h | | 29 | | | | | | 0 | 561 | 0 | 578 | 0 | 155,558 | 2,783 | R1 384-1 | sym | B05 | 311 |
| RPTEC | | 30 | | | | 30 | | 166 | 170 | 0 | 2,416 | 0 | 40,468 | 0 | R1 384-1 | sym | B06 | 320 |
| trachea-h | | 31 | | | | | | 0 | 5 | 0 | 2,843 | 618 | 126,079 | 3,124 | R1 384-1 | sym | B07 | 312 |
| HMEC | | 32 | | | | | | 0 | 0 | 0 | 0 | 0 | 14,267 | 0 | R1 384-1 | sym | B08 | 321 |
| uterus-h | | 33 | | | | | | 0 | 492 | 0 | 2,700 | 171 | 120,188 | 3,561 | R1 384-1 | sym | B09 | 313 |
| HCAEC | | 34 | | | | | | 495 | 0 | 0 | 0 | 300 | 11,188 | 0 | R1 384-1 | sym | B10 | 322 |
| Pancreas-h | | 35 | | | | | | 0 | 87 | 0 | 482 | 0 | 29,237 | 329 | R1 384-1 | sym | B11 | 314 |
| lymph node-h | | 36 | | | | | | 326 | 298 | 0 | 0 | 0 | 16,454 | 0 | R1 384-1 | sym | B12 | 323 |
| skeletal muscle-h | | 37 | | | | | | 242 | 0 | 0 | 1,216 | 0 | 164,981 | 412 | R1 384-1 | sym | B13 | 315 |
| fetal liver-h | | 38 | | | | | | 0 | 0 | 0 | 506 | 0 | 135,364 | 2,681 | R1 384-1 | sym | B14 | 324 |
| Heart-h | | 39 | | | | | | 500 | 94 | 0 | 2,052 | 0 | 84,824 | 394 | R1 384-1 | sym | B15 | 316 |
| thymus, h | | 40 | | | | | | 0 | 166 | 0 | 145 | 44 | 98,060 | 2,415 | R1 384-1 | sym | B16 | 325 |
| Duodenum-h | | 41 | | | | | | 717 | 113 | 0 | 2,188 | 421 | 70,179 | 559 | R1 384-1 | sym | B17 | 317 |
| Fetal brain-h | | 42 | | | | | | 490 | 0 | 0 | 0 | 0 | 32,869 | 359 | R1 384-1 | sym | B18 | 326 |
| Salivary gl.-h | | 43 | | | | | | 189 | 0 | 0 | 0 | 0 | 45,754 | 196 | R1 384-1 | sym | B19 | 318 |
| testis-h | | 44 | | | | | | 0 | 221 | 0 | 396 | 0 | 83,131 | 273 | R1 384-1 | sym | B20 | 327 |
| fetal kidney-h | | | | | 49 | | | 1,124 | 0 | 0 | 0 | 0 | 67,258 | 423 | R1 384-1 | asym | C01 | 92 |
| fetal liver-h | | | | | 51 | | | 443 | 0 | 0 | 0 | 0 | 18,565 | 208 | R1 384-1 | asym | C03 | 93 |
| fetal lung-h | | | | | 53 | | | 0 | 109 | 0 | 1,903 | 89 | 32,265 | 251 | R1 384-1 | asym | C05 | 94 |
| heart-h | | | | | 55 | | | 0 | 45 | 0 | 268 | 0 | 23,292 | 280 | R1 384-1 | asym | C07 | 95 |
| kidney-h | | | | | 57 | | | 0 | 0 | 0 | 45 | 0 | 84,812 | 1,129 | R1 384-1 | asym | C09 | 96 |
| lung-h | | | | | 59 | | | 0 | 183 | 0 | 0 | 0 | 80,505 | 199 | R1 384-1 | asym | C11 | 98 |
| lymph node-h | | | | | 61 | | | 45 | 0 | 0 | 0 | 40 | 143,506 | 3,584 | R1 384-1 | asym | C13 | 99 |
| WI-38 72h | | | | | 179 | | | 0 | 149 | 0 | 0 | 81 | 17,291 | 327 | R1 384-1 | asym | H11 | 85 |
| thymus-h | | | | | 193 | | | 0 | 0 | 0 | 0 | 659 | 60,103 | 252 | R1 384-1 | asym | I01 | 112 |
| HEPM 3d TGFB1 | | | | | 195 | | | 69 | 134 | 0 | 0 | 0 | 26,232 | 166 | R1 384-1 | asym | I03 | 70 |
| detergent + DNase | | | | | | | | | | | | | | | | | | |
| Salivary gl.-h | | | | | 197 | | | 0 | 0 | 0 | 278 | 0 | 11,196 | 143 | R1 384-1 | asym | I05 | 120 |
| testis-h | | | | | 199 | | | 0 | 86 | 0 | 0 | 17 | 32,630 | 0 | R1 384-1 | asym | I07 | 129 |
| Pancreas-h | | | | | 201 | | | 199 | 0 | 0 | 98 | 0 | 50,700 | 0 | R1 384-1 | asym | I09 | 116 |
| Skeletal muscle-h | | | | | 203 | | | 0 | 0 | 0 | 0 | 32 | 64,559 | 197 | R1 384-1 | asym | I11 | 117 |
| Duodenum-h | | | | | 205 | | | 0 | 0 | 0 | 0 | 0 | 33,654 | 0 | R1 384-1 | asym | I13 | 119 |
| HMEC | | | | | 209 | | | 777 | 102 | 0 | 0 | 206 | 101,094 | 3,485 | R1 384-1 | asym | I17 | 123 |
| fetal brain-h | | | | | 210 | | | 0 | 69 | 0 | 1,050 | 0 | 26,545 | 248 | R1 384-1 | asym | I18 | 191 |
| HCAEC | | | | | 211 | 211 | | 0 | 210 | 0 | 289 | 313 | 106,354 | 2,700 | R1 384-1 | asym | I19 | 124 |
| placenta-h | | | | | 212 | | | 0 | 296 | 0 | 0 | 0 | 16,093 | 223 | R1 384-1 | asym | I20 | 103 |
| fetal liver-h | | | | | 213 | | | 1,358 | 40 | 0 | 0 | 345 | 47,791 | 176 | R1 384-1 | asym | I21 | 126 |
| stomach-h | | | | | 214 | | | 0 | 0 | 0 | 151 | 0 | 26,452 | 196 | R1 384-1 | asym | I22 | 110 |
| Heart-h | | | | | 215 | | | 0 | 0 | 0 | 92 | 0 | 51,494 | 0 | R1 384-1 | asym | I23 | 118 |
| bladder-h | | | | | 222 | | | 0 | 140 | 0 | 0 | 0 | 39,930 | 0 | R1 384-1 | asym | J06 | 130 |
| HT372-normal | | | | | 234 | | | 0 | 50 | 0 | 0 | 0 | 19,299 | 10 | R1 384-1 | asym | J18 | 207 |
| HT382-normal | | | | | 266 | | | 615 | 78 | 0 | 0 | 0 | 86,365 | 202 | R1 384-1 | asym | L02 | 211 |
| HT392-normal | | | | | 268 | | | 131 | 0 | 0 | 0 | 286 | 30,692 | 361 | R1 384-1 | asym | L04 | 214 |
| HPAEC | | | | | 275 | 275 | | 0 | 0 | 0 | 563 | 0 | 40,511 | 422 | R1 384-1 | asym | L11 | 121 |
| adrenal gland-h | | | | | 277 | | | 2,553 | 446 | 166 | 27,369 | 329 | 1,532,778 | 4,110 | R1 384-1 | asym | L13 | 87 |
| bone marrow-h | | | | | 279 | | | 0 | 19 | 0 | 0 | 0 | 6,744 | 114 | R1 384-1 | asym | L15 | 88 |
| skeletal muscle-h | | | | | 290 | | | 0 | 45 | 0 | 1,545 | 0 | 102,189 | 1,579 | R1 384-1 | asym | M02 | 106 |
| small intestine-h | | | | | 292 | | | 0 | 0 | 0 | 0 | 0 | 40,631 | 318 | R1 384-1 | asym | M04 | 107 |
| spinal cord-h | | | | | 294 | | | 0 | 0 | 0 | 357 | 0 | 48,103 | 502 | R1 384-1 | asym | M06 | 108 |
| Spleen-h | | | | | 296 | | | 0 | 3 | 0 | 657 | 153 | 71,535 | 60 | R1 384-1 | asym | M08 | 109 |
| liver-h | | | | | 297 | | | 375 | 0 | 0 | 0 | 0 | 88,510 | 74 | R1 384-1 | asym | M09 | 97 |
| testis-h | | | | | 298 | | | 422 | 0 | 0 | 640 | 0 | 167,195 | 2,423 | R1 384-1 | asym | M10 | 111 |
| bladder-h | | | | | 302 | | | 38 | 311 | 0 | 2,206 | 0 | 74,917 | 825 | R1 384-1 | sym | M14 | 328 |
| mammary gland-h | | | | | 303 | | | 221 | 0 | 0 | 794 | 117 | 48,744 | 0 | R1 384-1 | asym | M15 | 100 |
| pancreas-h | | | | | 305 | | | 678 | 16 | 0 | 817 | 473 | 127,476 | 342 | R1 384-1 | asym | M17 | 101 |
| pituitary gland-h | | | | | 307 | | | 0 | 0 | 0 | 0 | 0 | 20,657 | 0 | R1 384-1 | asym | M19 | 102 |
| prostate, h | | | | | 309 | | | 0 | 634 | 0 | 297 | 2,243 | 148,345 | 3,258 | R1 384-1 | asym | M21 | 104 |
| salivary gl-h | | | | | 311 | | | 455 | 0 | 0 | 1,109 | 0 | 18,958 | 0 | | | | |
| SA-OS (Mundy) | | | | | | | | | | | | | | | R1 384-1 | asym | M23 | 105 |
| poly A+ | | | | | | | | | | | | | | | | | | |
| thyroid gland-h | | | | | 314 | | | 0 | 0 | 0 | 0 | 0 | 127,246 | 72 | R1 384-1 | asym | N02 | 113 |
| trachea-h | | | | | 316 | | | 0 | 125 | 0 | 16 | 172 | 80,358 | 0 | R1 384-1 | asym | N04 | 114 |
| uterus-h | | | | | 318 | | | 19 | 0 | 0 | 1,641 | 0 | 73,044 | 1,084 | R1 384-1 | asym | N06 | 115 |
| HEPM 3d untreated | | | | | 320 | | | 1,065 | 18 | 0 | 0 | 510 | 61,981 | 0 | R1 384-1 | asym | N08 | 71 |
| HT149-normal | | | | | 321 | | | 528 | 0 | 0 | 0 | 0 | 56,128 | 155 | R1 384-1 | asym | N09 | 163 |
| thymus, h | | | | | 326 | | | 0 | 0 | 0 | 0 | 0 | 51,419 | 0 | R1 384-1 | asym | N14 | 127 |
| HT398-normal | | | | | 327 | | | 551 | 431 | 0 | 742 | 0 | 32,502 | 24 | R1 384-1 | asym | N15 | 219 |
| Fetal brain-h | | | | | 328 | | | 734 | 0 | 0 | 1,133 | 0 | 166,205 | 3,822 | R1 384-1 | asym | N16 | 128 |
| h adult SMC | | | | | 330 | | | 0 | 0 | 0 | 0 | 0 | 23,012 | 164 | R1 384-1 | asym | N18 | 75 |
| Oct. 21, 1992 #17 | | | | | | | | | | | | | | | | | | |
| lymph node-h | | | | | 332 | | | 95 | 0 | 0 | 0 | 67 | 22,358 | 0 | R1 384-1 | asym | N20 | 125 |
| RPTEC | | | | | 334 | 334 | | 1,186 | 189 | 0 | 223 | 0 | 96,951 | 2,705 | R1 384-1 | asym | N22 | 122 |
| brain-h | | | | | 342 | | | 0 | 204 | 0 | 0 | 716 | 51,925 | 0 | R1 384-1 | asym | O06 | 89 |
| cerebellum-h | | | | | 344 | | | 0 | 0 | 0 | 40 | 0 | 31,053 | 0 | R1 384-1 | asym | O08 | 90 |
| HT157-normal | | | | | 361 | | | 0 | 285 | 0 | 0 | 0 | 81,782 | 1 | R1 384-1 | asym | P01 | 168 |
| HT213-normal | | | | | 363 | | | 1,947 | 45 | 0 | 15 | 0 | 20,122 | 0 | R1 384-1 | asym | P03 | 181 |
| HT218-normal | | | | | 365 | | | 0 | 0 | 0 | 0 | 0 | 13,192 | 0 | R1 384-1 | asym | P05 | 183 |
| HeLa-1 | | | | | | | HPV E6 | 188 | 27 | 0 | 17,506 | 191 | 555,591 | 1,745 | R2 384-1 | sym | C10 | 394 |
| HeLa-2 | | | | | | | HPV E6 | 362 | 758 | 2 | 52,193 | 0 | 679,609 | 1,613 | R2 384-1 | sym | F18 | 395 |
| HeLa-3 | | | | | | | HPV E6 | 1,051 | 0 | 67 | 19,138 | 0 | 443,018 | 1,179 | R2 384-1 | sym | C12 | 396 |
| HeLa-4 | | | | | | | HPV E6 | 699 | 387 | 0 | 11,837 | 0 | 636,546 | 1,133 | R2 384-1 | sym | C14 | 397 |
| HeLa-5 | | | | | | | HPV E6 | 1,571 | 0 | 600 | 40,632 | 0 | 740,204 | 1,934 | R2 384-1 | sym | C16 | 398 |
| HeLa-6 | | | | | | | HPV E6 | 0 | 0 | 215 | 40,279 | 174 | 849,472 | 3,502 | R2 384-1 | sym | C18 | 399 |
| HeLa-7 | | | | | | | HPV E6 | 233 | 0 | 183 | 39,006 | 81 | 914,876 | 2,315 | R2 384-1 | sym | C20 | 400 |
| HeLa-8 | | | | | | | HPV E6 | 0 | 0 | 0 | 30,268 | 0 | 967,084 | 1,982 | R2 384-1 | sym | C22 | 401 |
| ADR-RES-1 | | | | | | | mutant | 540 | 168 | 0 | 48,622 | 0 | 921,921 | 922 | R2 384-1 | sym | C17 | 341 |
| ADR-RES-2 | | | | | | | mutant | 76 | 0 | 0 | 18,959 | 0 | 1,010,246 | 591 | R2 384-1 | sym | C19 | 342 |
| ADR-RES-3 | | | | | | | mutant | 334 | 344 | 0 | 13,658 | 0 | 359,976 | 383 | R2 384-1 | sym | C21 | 343 |
| ADR-RES-4 | | | | | | | mutant | 0 | 0 | 0 | 8,302 | 0 | 442,971 | 11 | R2 384-1 | sym | C23 | 344 |
| ADR-RES-5 | | | | | | | mutant | 306 | 0 | 164 | 20,910 | 0 | 544,895 | 130 | R2 384-1 | sym | C02 | 345 |
| ADR-RES-6 | | | | | | | mutant | 140 | 0 | 0 | 26,446 | 0 | 601,648 | 468 | R2 384-1 | sym | C04 | 346 |
| ADR-RES-7 | | | | | | | mutant | 0 | 661 | 0 | 1,745 | 429 | 85,849 | 3,468 | R2 384-1 | sym | C06 | 347 |
| ADR-RES-8 | | | | | | | mutant | 0 | 283 | 0 | 3,363 | 0 | 5,866 | 0 | R2 384-1 | sym | C08 | 348 |
| C33A-1 | | | | | | | mutant | 0 | 0 | 0 | 3,630 | 0 | 458,295 | 578 | R2 384-1 | sym | D08 | 349 |
| C33A-2 | | | | | | | mutant | 0 | 0 | 0 | 13,815 | 0 | 389,598 | 609 | R2 384-1 | sym | D10 | 350 |
| C33A-3 | | | | | | | mutant | 4,866 | 0 | 104 | 9,604 | 0 | 379,820 | 824 | R2 384-1 | sym | D12 | 351 |
| C33A-4 | | | | | | | mutant | 89 | 0 | 126 | 8,326 | 42 | 498,796 | 1,112 | R2 384-1 | sym | D14 | 352 |
| C33A-5 | | | | | | | mutant | 0 | 0 | 0 | 34,820 | 0 | 644,383 | 890 | R2 384-1 | sym | D16 | 353 |
| C33A-6 | | | | | | | mutant | 0 | 0 | 0 | 690 | 0 | 678,800 | 0 | R2 384-1 | sym | D18 | 354 |
| C33A-7 | | | | | | | mutant | 0 | 0 | 149 | 18,136 | 0 | 785,878 | 908 | R2 384-1 | sym | D20 | 355 |
| C33A-8 | | | | | | | mutant | 21 | 0 | 173 | 42,512 | 0 | 1,172,556 | 2,189 | R2 384-1 | sym | D22 | 356 |
| EKVX-1 | | | | | | | mutant | 675 | 0 | 0 | 27,186 | 0 | 269,251 | 221 | R2 384-1 | sym | F02 | 370 |
| EKVX-2 | | | | | | | mutant | 0 | 0 | 92 | 69,264 | 272 | 498,108 | 0 | R2 384-1 | sym | F04 | 371 |
| EKVX-3 | | | | | | | mutant | 0 | 4 | 0 | 93,678 | 0 | 429,070 | 340 | R2 384-1 | sym | F06 | 372 |
| EKVX-4 | | | | | | | mutant | 577 | 0 | 71 | 140,696 | 344 | 429,429 | 819 | R2 384-1 | sym | F08 | 373 |
| EKVX-5 | | | | | | | mutant | 29 | 0 | 0 | 34,966 | 0 | 813,745 | 1,003 | R2 384-1 | sym | A17 | 374 |
| EKVX-6 | | | | | | | mutant | 6 | 0 | 98 | 7,689 | 79 | 459,086 | 799 | R2 384-1 | sym | A19 | 375 |
| EKVX-7 | | | | | | | mutant | 705 | 0 | 564 | 41,327 | 393 | 1,353,897 | 2,803 | R2 384-1 | sym | A21 | 376 |
| EKVX-8 | | | | | | | mutant | 192 | 242 | 0 | 31,342 | 0 | 397,494 | 1,472 | R2 384-1 | sym | F10 | 377 |
| H1299-1 | | | | | | | mutant | 1,259 | 0 | 62 | 26,353 | 0 | 501,247 | 1,055 | R2 384-1 | sym | D15 | 378 |
| H1299-2 | | | | | | | mutant | 0 | 327 | 37 | 13,882 | 0 | 712,338 | 1,004 | R2 384-1 | sym | D17 | 379 |
| H1299-3 | | | | | | | mutant | 0 | 121 | 16 | 36,682 | 0 | 778,091 | 2,440 | R2 384-1 | sym | D19 | 380 |
| H1299-4 | | | | | | | mutant | 172 | 0 | 22 | 57,330 | 669 | 957,276 | 2,317 | R2 384-1 | sym | D21 | 381 |
| H1299-5 | | | | | | | mutant | 0 | 0 | 92 | 18,628 | 0 | 865,896 | 1,039 | R2 384-1 | sym | D23 | 382 |
| H1299-6 | | | | | | | mutant | 0 | 79 | 52 | 17,264 | 172 | 338,356 | 412 | R2 384-1 | sym | D02 | 383 |
| H1299-7 | | | | | | | mutant | 147 | 0 | 0 | 12,801 | 0 | 523,628 | 0 | R2 384-1 | sym | D04 | 384 |
| H1299-8 | | | | | | | mutant | 2,032 | 0 | 0 | 21,626 | 553 | 519,154 | 449 | R2 384-1 | sym | D06 | 385 |
| HT29-1 | | | | | | | mutant | 817 | 0 | 85 | 60,106 | 0 | 859,216 | 1,413 | R2 384-1 | sym | A14 | 409 |
| HT29-2 | | | | | | | mutant | 1,145 | 298 | 16 | 62,721 | 0 | 440,697 | 1,568 | R2 384-1 | sym | A16 | 410 |
| HT29-3 | | | | | | | mutant | 833 | 228 | 132 | 85,651 | 228 | 700,898 | 958 | R2 384-1 | sym | A18 | 411 |
| HT29-4 | | | | | | | mutant | 120 | 0 | 258 | 102,716 | 604 | 1,046,464 | 1,512 | R2 384-1 | sym | A20 | 412 |
| HT29-5 | | | | | | | mutant | 810 | 0 | 0 | 137,429 | 0 | 1,522,264 | 3,276 | R2 384-1 | sym | A22 | 413 |
| HT29-6 | | | | | | | mutant | 3,401 | 128 | 154 | 131,385 | 568 | 1,157,087 | 2,892 | R2 384-1 | sym | A24 | 414 |
| HT29-7 | | | | | | | mutant | 0 | 0 | 25 | 21,556 | 455 | 359,692 | 523 | R2 384-1 | sym | B01 | 415 |
| HT29-8 | | | | | | | mutant | 0 | 0 | 227 | 34,495 | 11 | 539,304 | 1,099 | R2 384-1 | sym | B03 | 416 |
| OVCAR-5-1 | | | | | | | mutant | 166 | 0 | 0 | 46,515 | 403 | 407,563 | 1,031 | R2 384-1 | sym | B12 | 454 |
| OVCAR-5-2 | | | | | | | mutant | 0 | 0 | 81 | 22,899 | 15 | 550,448 | 653 | R2 384-1 | sym | B14 | 455 |
| OVCAR-5-3 | | | | | | | mutant | 66 | 344 | 186 | 67,219 | 0 | 386,258 | 33 | R2 384-1 | sym | B16 | 456 |
| OVCAR-5-4 | | | | | | | mutant | 0 | 81 | 0 | 28,479 | 1 | 460,438 | 2,500 | R2 384-1 | sym | B18 | 457 |
| OVCAR-5-5 | | | | | | | mutant | 16 | 314 | 0 | 52,491 | 140 | 607,764 | 883 | R2 384-1 | sym | H03 | 458 |
| OVCAR-5-6 | | | | | | | mutant | 0 | 144 | 41 | 5,128 | 0 | 652,947 | 91 | R2 384-1 | sym | B22 | 459 |
| OVCAR-5-7 | | | | | | | mutant | 1,388 | 743 | 379 | 73,397 | 0 | 2,126,515 | 1,252 | R2 384-1 | sym | B24 | 460 |
| OVCAR-5-8 | | | | | | | mutant | 687 | 0 | 0 | 72,830 | 1,009 | 411,234 | 1,283 | R2 384-1 | sym | F16 | 461 |
| SF-268-1 | | | | | | | mutant | 454 | 0 | 0 | 46,444 | 169 | 1,247,938 | 3,199 | R2 384-1 | sym | E24 | 462 |
| SF-268-2 | | | | | | | mutant | 175 | 0 | 1 | 31,761 | 0 | 294,676 | 519 | R2 384-1 | sym | F01 | 463 |
| SF-268-3 | | | | | | | mutant | 35 | 0 | 211 | 26,186 | 0 | 442,870 | 212 | R2 384-1 | sym | F03 | 464 |
| SF-268-4 | | | | | | | mutant | 178 | 184 | 204 | 51,244 | 0 | 399,799 | 568 | R2 384-1 | sym | F05 | 465 |
| SF-268-5 | | | | | | | mutant | 0 | 816 | 0 | 56,103 | 0 | 471,620 | 0 | R2 384-1 | sym | F07 | 466 |
| SF-268-6 | | | | | | | mutant | 0 | 36 | 0 | 25,570 | 0 | 394,514 | 619 | R2 384-1 | sym | F09 | 467 |
| SF-268-7 | | | | | | | mutant | 850 | 143 | 0 | 40,201 | 429 | 347,362 | 1,837 | R2 384-1 | sym | F11 | 468 |
| SF-268-8 | | | | | | | mutant | 2,391 | 138 | 160 | 67,712 | 12 | 463,334 | 1,254 | R2 384-1 | sym | F13 | 469 |
| SW480-1 | | | | | | | mutant | 0 | 0 | 0 | 26 | 0 | 13,937 | 30 | R2 384-1 | sym | C24 | 478 |
| SW480-2 | | | | | | | mutant | 0 | 255 | 0 | 1,000 | 79 | 33,878 | 1,032 | R2 384-1 | sym | D01 | 479 |
| SW480-3 | | | | | | | mutant | 470 | 0 | 0 | 76,090 | 0 | 1,460,381 | 3,005 | R2 384-1 | sym | D03 | 480 |
| SW480-4 | | | | | | | mutant | 79 | 0 | 0 | 45,130 | 226 | 423,651 | 537 | R2 384-1 | sym | D05 | 481 |
| SW480-5 | | | | | | | mutant | 260 | 85 | 206 | 33,127 | 0 | 392,824 | 235 | R2 384-1 | sym | D07 | 482 |
| SW480-6 | | | | | | | mutant | 733 | 0 | 0 | 11,557 | 0 | 536,855 | 116 | R2 384-1 | sym | D09 | 483 |
| SW480-7 | | | | | | | mutant | 1,608 | 0 | 0 | 38,957 | 0 | 729,185 | 983 | R2 384-1 | sym | D11 | 484 |
| SW480-8 | | | | | | | mutant | 183 | 0 | 0 | 27,056 | 0 | 555,267 | 1,473 | R2 384-1 | sym | D13 | 485 |
| U2OS-1 | | | | | | | mutant | 1,263 | 125 | 0 | 78,276 | 1,905 | 1,630,846 | 4,317 | R2 384-1 | sym | D24 | 486 |
| U2OS-2 | | | | | | | mutant | 641 | 327 | 202 | 29,915 | 0 | 432,337 | 972 | R2 384-1 | sym | E01 | 487 |
| U2OS-3 | | | | | | | mutant | 573 | 0 | 1 | 40,362 | 127 | 406,461 | 412 | R2 384-1 | sym | E03 | 488 |
| U2OS-4 | | | | | | | mutant | 1,545 | 0 | 162 | 10,515 | 106 | 399,039 | 738 | R2 384-1 | sym | E05 | 489 |
| U2OS-5 | | | | | | | mutant | 679 | 0 | 94 | 37,160 | 0 | 491,594 | 827 | R2 384-1 | sym | E07 | 490 |
| U2OS-6 | | | | | | | mutant | 0 | 294 | 108 | 5,845 | 3,862 | 371,040 | 1,175 | R2 384-1 | sym | E09 | 491 |
| U2OS-7 | | | | | | | mutant | 0 | 0 | 0 | 22,921 | 17 | 500,363 | 1,551 | R2 384-1 | sym | E11 | 492 |
| U2OS-8 | | | | | | | mutant | 0 | 0 | 0 | 16,035 | 0 | 472,901 | 1,979 | R2 384-1 | sym | E13 | 493 |
| A549-1 | | | | | | | wt | 0 | 0 | 112 | 54,563 | 0 | 546,984 | 523 | R2 384-1 | sym | A01 | 333 |
| A549-2 | | | | | | | wt | 1,553 | 0 | 0 | 25,728 | 192 | 490,791 | 367 | R2 384-1 | sym | A03 | 334 |
| A549-3 | | | | | | | wt | 0 | 0 | 0 | 8,583 | 0 | 526,257 | 173 | R2 384-1 | sym | A05 | 335 |
| A549-4 | | | | | | | wt | 56 | 118 | 92 | 27,986 | 170 | 637,978 | 439 | R2 384-1 | sym | A07 | 336 |
| A549-5 | | | | | | | wt | 0 | 119 | 0 | 56,834 | 0 | 470,171 | 657 | R2 384-1 | sym | A09 | 337 |
| A549-6 | | | | | | | wt | 0 | 0 | 203 | 2,773 | 0 | 560,302 | 671 | R2 384-1 | sym | A11 | 338 |
| A549-7 | | | | | | | wt | 240 | 0 | 188 | 32,976 | 0 | 658,479 | 1,455 | R2 384-1 | sym | A13 | 339 |
| A549-8 | | | | | | | wt | 57 | 157 | 1 | 74,102 | 243 | 409,754 | 1,458 | R2 384-1 | sym | A15 | 340 |
| HCT-116-1 | | | | | | | wt | 0 | 0 | 0 | 100,001 | 85 | 2,916,547 | 3,159 | R2 384-1 | sym | A23 | 386 |
| HCT-116-2 | | | | | | | wt | 0 | 18 | 4 | 145,047 | 0 | 386,158 | 1,870 | R2 384-1 | sym | F12 | 387 |
| HCT-116-3 | | | | | | | wt | 665 | 0 | 0 | 17,785 | 159 | 427,289 | 313 | R2 384-1 | sym | A02 | 388 |
| HCT-116-4 | | | | | | | wt | 275 | 0 | 0 | 38,703 | 0 | 386,481 | 198 | R2 384-1 | sym | A04 | 389 |
| HCT-116-5 | | | | | | | wt | 556 | 179 | 38 | 37,110 | 85 | 505,899 | 955 | R2 384-1 | sym | A06 | 390 |
| HCT-116-6 | | | | | | | wt | 754 | 0 | 105 | 61,500 | 0 | 498,369 | 488 | R2 384-1 | sym | A08 | 391 |
| HCT-116-7 | | | | | | | wt | 190 | 187 | 47 | 42,722 | 0 | 416,088 | 1,069 | R2 384-1 | sym | A10 | 392 |
| HCT-116-8 | | | | | | | wt | 783 | 0 | 49 | 48,607 | 133 | 555,183 | 1,362 | R2 384-1 | sym | A12 | 393 |
| Hs68-1 | | | | | | | wt | 0 | 0 | 50 | 21,139 | 0 | 412,159 | 1,066 | R2 384-1 | sym | E15 | 402 |
| Hs68-2 | | | | | | | wt | 0 | 84 | 369 | 52,149 | 175 | 562,011 | 1,788 | R2 384-1 | sym | E17 | 403 |
| Hs68-3 | | | | | | | wt | 21 | 0 | 0 | 52,724 | 119 | 832,196 | 1,856 | R2 384-1 | sym | E19 | 404 |
| Hs68-4 | | | | | | | wt | 1,647 | 0 | 0 | 104,514 | 40 | 1,087,919 | 3,110 | R2 384-1 | sym | E21 | 405 |
| Hs68-6 | | | | | | | wt | 0 | 0 | 47 | 6,341 | 425 | 271,530 | 521 | R2 384-1 | sym | E02 | 406 |
| Hs68-7 | | | | | | | wt | 367 | 0 | 0 | 25,147 | 0 | 491,608 | 246 | R2 384-1 | sym | E04 | 407 |
| Hs68-8 | | | | | | | wt | 0 | 16 | 0 | 30,704 | 0 | 498,513 | 192 | R2 384-1 | sym | E06 | 408 |
| MCF-7-1 | | | | | | | wt | 0 | 0 | 0 | 7,662 | 0 | 604,049 | 344 | R2 384-1 | sym | C01 | 417 |
| MCF-7-2 | | | | | | | wt | 1,092 | 0 | 141 | 26,075 | 0 | 662,630 | 341 | R2 384-1 | sym | C03 | 418 |
| MCF-7-3 | | | | | | | wt | 1,349 | 0 | 341 | 17,550 | 0 | 632,258 | 621 | R2 384-1 | sym | C05 | 419 |
| MCF-7-4 | | | | | | | wt | 275 | 6 | 0 | 20,701 | 0 | 661,141 | 1,174 | R2 384-1 | sym | C07 | 420 |
| MCF-7-5 | | | | | | | wt | 0 | 0 | 0 | 8,565 | 237 | 484,097 | 737 | R2 384-1 | sym | C09 | 421 |
| MCF-7-6 | | | | | | | wt | 0 | 0 | 0 | 20,587 | 386 | 795,802 | 1,446 | R2 384-1 | sym | C11 | 422 |
| MCF-7-7 | | | | | | | wt | 0 | 0 | 0 | 0 | 357 | 33,599 | 0 | R2 384-1 | sym | C13 | 423 |
| MCF-7-8 | | | | | | | wt | 560 | 0 | 0 | 2,415 | 460 | 76,817 | 535 | R2 384-1 | sym | C15 | 424 |
| OVCAR-4-1 | | | | | | | wt | 1,159 | 0 | 126 | 65,996 | 43 | 777,026 | 2,150 | R2 384-1 | sym | B21 | 446 |
| OVCAR-4-2 | | | | | | | wt | 0 | 479 | 0 | 48,716 | 0 | 3,789,726 | 5,436 | R2 384-1 | sym | B23 | 447 |
| OVCAR-4-3 | | | | | | | wt | 0 | 314 | 77 | 29,888 | 0 | 417,174 | 388 | R2 384-1 | sym | B02 | 448 |
| OVCAR-4-4 | | | | | | | wt | 0 | 118 | 0 | 10,373 | 0 | 477,283 | 117 | R2 384-1 | sym | B04 | 449 |
| OVCAR-4-5 | | | | | | | wt | 396 | 123 | 89 | 38,345 | 0 | 581,094 | 506 | R2 384-1 | sym | B06 | 450 |
| OVCAR-4-6 | | | | | | | wt | 462 | 56 | 0 | 41,745 | 0 | 600,108 | 457 | R2 384-1 | sym | B08 | 451 |
| OVCAR-4-7 | | | | | | | wt | 59 | 0 | 208 | 7,508 | 356 | 427,310 | 554 | R2 384-1 | sym | B10 | 452 |
| OVCAR-4-8 | | | | | | | wt | 278 | 0 | 121 | 88,152 | 0 | 471,191 | 924 | R2 384-1 | sym | H01 | 453 |
| SF539-1 | | | | | | | wt | 0 | 283 | 92 | 11,389 | 0 | 509,238 | 91 | R2 384-1 | sym | B05 | 470 |
| SF539-2 | | | | | | | wt | 277 | 145 | 0 | 9,827 | 11 | 651,624 | 427 | R2 384-1 | sym | B07 | 471 |
| SF539-3 | | | | | | | wt | 1,599 | 0 | 0 | 21,929 | 0 | 547,965 | 799 | R2 384-1 | sym | B09 | 472 |
| SF539-4 | | | | | | | wt | 0 | 179 | 0 | 19,159 | 0 | 449,192 | 668 | R2 384-1 | sym | B11 | 473 |
| SF539-5 | | | | | | | wt | 85 | 0 | 323 | 23,329 | 0 | 516,343 | 319 | R2 384-1 | sym | B13 | 474 |
| SF539-6 | | | | | | | wt | 0 | 108 | 0 | 7,791 | 0 | 662,214 | 668 | R2 384-1 | sym | B15 | 475 |
| SF539-7 | | | | | | | wt | 0 | 0 | 290 | 17,423 | 0 | 574,107 | 1,461 | R2 384-1 | sym | B17 | 476 |
| SF539-8 | | | | | | | wt | 0 | 58 | 0 | 38,279 | 0 | 550,442 | 1,574 | R2 384-1 | sym | B19 | 477 |
| WI 38-1 | | | | | | | wt | 391 | 90 | 25 | 54,921 | 0 | 498,076 | 672 | R2 384-1 | sym | E08 | 494 |
| WI 38-2 | | | | | | | wt | 725 | 671 | 339 | 93,773 | 270 | 339,104 | 2,179 | R2 384-1 | sym | E10 | 495 |
| WI 38-3 | | | | | | | wt | 1,101 | 524 | 65 | 40,084 | 0 | 414,523 | 945 | R2 384-1 | sym | E12 | 496 |
| WI 38-4 | | | | | | | wt | 6 | 752 | 0 | 37,054 | 0 | 620,563 | 661 | R2 384-1 | sym | E14 | 497 |
| WI 38-5 | | | | | | | wt | 0 | 289 | 0 | 7,149 | 0 | 552,397 | 1,748 | R2 384-1 | sym | E16 | 498 |
| WI 38-6 | | | | | | | wt | 161 | 0 | 0 | 10,543 | 0 | 559,003 | 724 | R2 384-1 | sym | E18 | 499 |
| WI 38-7 | | | | | | | wt | 385 | 63 | 92 | 31,968 | 0 | 832,476 | 1,298 | R2 384-1 | sym | E20 | 500 |
| WI 38-8 | | | | | | | wt | 80 | 157 | 0 | 5,997 | 0 | 1,071,262 | 1,559 | R2 384-1 | sym | E22 | 501 |
| HELA-2h-031899 | | | | 79 | | | | 533 | 0 | 0 | 680 | 127 | 61,571 | 1,378 | R1 384-1 | asym | D07 | 148 |
| HELA-4h-031899 | | | | 81 | | | | 0 | 0 | 0 | 1,627 | 0 | 96,196 | 929 | R1 384-1 | asym | D09 | 149 |
| HELA-9h-031899 | | | | 83 | | | | 0 | 232 | 0 | 174 | 0 | 91,313 | 810 | R1 384-1 | asym | D11 | 152 |
| HELA-0h-031899 | | | | 86 | | | | 0 | 0 | 0 | 293 | 0 | 41,904 | 164 | R1 384-1 | asym | D14 | 147 |
| HELA-6h-031899 | | | | 88 | | | | 305 | 0 | 0 | 462 | 0 | 56,525 | 291 | R1 384-1 | asym | D16 | 150 |
| HELA-8h-031899 | | | | 90 | | | | 0 | 0 | 0 | 0 | 0 | 80,592 | 531 | R1 384-1 | asym | D18 | 151 |
| HELA-10h-031899 | | | | 92 | | | | 0 | 0 | 0 | 2,217 | 0 | 50,148 | 0 | R1 384-1 | asym | D20 | 153 |
| HELA-11h-031899 | | | | 94 | | | | 0 | 0 | 0 | 0 | 0 | 45,767 | 0 | R1 384-1 | asym | D22 | 154 |
| HELA-12h-031899 | | | | 96 | | | | 496 | 0 | 0 | 231 | 0 | 43,735 | 297 | R1 384-1 | asym | D24 | 155 |
| NCI-H322M | | | | 146 | | | | 0 | 138 | 0 | 1,805 | 0 | 69,902 | 1,926 | R1 384-1 | asym | G02 | 5 |
| NCI-H460 | | | | 148 | | | | 528 | 0 | 0 | 0 | 432 | 41,427 | 0 | R1 384-1 | asym | G04 | 6 |
| NCI-H522 | | | | 150 | | | | 0 | 101 | 0 | 0 | 960 | 64,259 | 1,627 | R1 384-1 | asym | G06 | 7 |
| SNB-19 | | | | 152 | | | | 515 | 0 | 0 | 0 | 217 | 15,805 | 214 | R1 384-1 | asym | G08 | 16 |
| SNB-75 | | | | 154 | | | | 10 | 0 | 0 | 1,207 | 0 | 36,729 | 0 | R1 384-1 | asym | G10 | 17 |
| SF-268 | | | | 156 | | | | 405 | 0 | 0 | 0 | 0 | 11,197 | 354 | R1 384-1 | asym | G12 | 19 |
| SF-295 | | | | 158 | | | | 612 | 630 | 0 | 760 | 532 | 43,210 | 1,364 | R1 384-1 | asym | G14 | 20 |
| CCRF-CEM | | | | 160 | | | | 0 | 0 | 0 | 599 | 0 | 2,830 | 40 | R1 384-1 | asym | G16 | 22 |
| DU-145 | | | | 162 | | | | 148 | 0 | 0 | 0 | 0 | 55,584 | 970 | R1 384-1 | asym | G18 | 28 |
| HCT 116 | | | | 164 | | | | 876 | 0 | 0 | 171 | 604 | 48,404 | 640 | R1 384-1 | asym | G20 | 31 |
| Caki-1 | | | | 166 | | | | 1,789 | 63 | 0 | 195 | 357 | 9,678 | 0 | R1 384-1 | asym | G22 | 39 |
| 786-0 | | | | 168 | | | | 847 | 114 | 0 | 558 | 0 | 5,791 | 48 | R1 384-1 | asym | G24 | 42 |
| T-47D | | | | 169 | | | | 404 | 55 | 0 | 1,859 | 0 | 13,446 | 531 | R1 384-1 | asym | H01 | 58 |
| CRL1441 RNA 8/30 | | | | 181 | | | | 1,738 | 0 | 0 | 0 | 590 | 4,867 | 0 | R1 384-1 | asym | H13 | 79 |
| 781T untreated + | | | | 183 | | | | 1,066 | 16 | 0 | 0 | 52 | 4,220 | 168 | R1 384-1 | asym | H15 | 73 |
| DNase | | | | | | | | | | | | | | | | | | |
| KB poly A+ | | | | 194 | | | | 452 | 357 | 0 | 0 | 219 | 43,402 | 0 | R1 384-1 | asym | I02 | 59 |
| HOS poly A+ | | | | 196 | | | | 0 | 0 | 0 | 0 | 904 | 58,885 | 468 | R1 384-1 | asym | I04 | 62 |
| ACHN | | | | 198 | | | | 426 | 236 | 0 | 193 | 3,740 | 67,534 | 748 | R1 384-1 | asym | I06 | 41 |
| UACC-62 | | | | 200 | | | | 0 | 282 | 0 | 459 | 7 | 12,401 | 0 | R1 384-1 | asym | I08 | 49 |
| MCF-7/ADR-RES | | | | 202 | | | | 776 | 0 | 0 | 209 | 0 | 44,167 | 0 | R1 384-1 | asym | I10 | 53 |
| UTOS (Mundy) | | | | 204 | | | | 0 | 143 | 0 | 373 | 359 | 13,394 | 115 | R1 384-1 | asym | I12 | 65 |
| poly a+ | | | | | | | | | | | | | | | | | | |
| WISH (Collagen) | | | | 206 | | | | 1,552 | 0 | 0 | 0 | 0 | 3,311 | 0 | R1 384-1 | asym | I14 | 66 |
| poly A+ | | | | | | | | | | | | | | | | | | |
| 458 medullo mRNA | | | | 208 | | | | 0 | 290 | 0 | 4,761 | 0 | 38,074 | 0 | R1 384-1 | asym | I16 | 69 |
| CCL137 RNA | | | | 218 | | | | 0 | 0 | 0 | 392 | 65 | 35,013 | 425 | R1 384-1 | asym | J02 | 77 |
| Mar. 21, 1988 | | | | | | | | | | | | | | | | | | |
| WI-38 72h 0.5% | | | | 219 | | | | 0 | 0 | 0 | 0 | 0 | 43,199 | 0 | R1 384-1 | asym | J03 | 86 |
| FBS, 24h 10% FBS | | | | | | | | | | | | | | | | | | |
| CRL 1441 + TPA | | | | 220 | | | | 0 | 56 | 0 | 386 | 0 | 13,416 | 59 | R1 384-1 | asym | J04 | 78 |
| (24h) 8/30 | | | | | | | | | | | | | | | | | | |
| HOP-92 | | | | 241 | | | | 0 | 23 | 0 | 0 | 0 | 22,647 | 186 | R1 384-1 | asym | K01 | 1 |
| MOLT-4 | | | | 242 | | | | 0 | 92 | 0 | 309 | 26 | 21,250 | 434 | R1 384-1 | asym | K02 | 24 |
| EKVX | | | | 243 | | | | 0 | 250 | 0 | 960 | 0 | 77,320 | 2,875 | R1 384-1 | asym | K03 | 2 |
| HL-60 | | | | 244 | | | | 0 | 0 | 0 | 908 | 480 | 24,392 | 0 | R1 384-1 | asym | K04 | 25 |
| NCI-H23 | | | | 245 | | | | 1,129 | 0 | 0 | 211 | 0 | 93,263 | 2,462 | R1 384-1 | asym | K05 | 3 |
| RPMI 8226 | | | | 246 | | | | 0 | 0 | 0 | 799 | 0 | 87,784 | 1,931 | R1 384-1 | asym | K06 | 26 |
| A549/ATCC | | | | 247 | | | | 226 | 0 | 0 | 0 | 211 | 5,669 | 0 | R1 384-1 | asym | K07 | 8 |
| SR | | | | 248 | | | | 329 | 0 | 0 | 0 | 0 | 15,586 | 296 | R1 384-1 | asym | K08 | 27 |
| OVCAR-3 | | | | 249 | | | | 112 | 0 | 0 | 0 | 594 | 15,287 | 240 | R1 384-1 | asym | K09 | 10 |
| HCT-15 | | | | 250 | | | | 284 | 387 | 0 | 2,254 | 94 | 30,611 | 211 | R1 384-1 | asym | K10 | 34 |
| OVCAR-4 | | | | 251 | | | | 0 | 278 | 0 | 0 | 0 | 4,121 | 166 | R1 384-1 | asym | K11 | 11 |
| UO-31 | | | | 252 | | | | 0 | 0 | 0 | 0 | 30 | 7,831 | 96 | R1 384-1 | asym | K12 | 36 |
| OVCAR-5 | | | | 253 | | | | 181 | 265 | 0 | 1,579 | 756 | 28,038 | 1,658 | R1 384-1 | asym | K13 | 12 |
| SN12C | | | | 254 | | | | 0 | 151 | 0 | 0 | 0 | 5,978 | 0 | R1 384-1 | asym | K14 | 37 |
| OVCAR-8 | | | | 255 | | | | 0 | 7 | 0 | 952 | 591 | 67,456 | 1,954 | R1 384-1 | asym | K15 | 13 |
| LOX IMVI | | | | 256 | | | | 239 | 624 | 0 | 2,645 | 954 | 25,761 | 1,176 | R1 384-1 | asym | K16 | 44 |
| IGROV1 | | | | 257 | | | | 0 | 0 | 0 | 1,111 | 3 | 56,485 | 1,865 | R1 384-1 | asym | K17 | 14 |
| SK-MEL-2 | | | | 258 | | | | 0 | 0 | 0 | 0 | 0 | 2,107 | 619 | R1 384-1 | asym | K18 | 46 |
| SK-OV-3 | | | | 259 | | | | 350 | 0 | 0 | 0 | 0 | 3,010 | 95 | R1 384-1 | asym | K19 | 15 |
| SK-MEL-5 | | | | 260 | | | | 0 | 0 | 0 | 0 | 0 | 1,573 | 0 | R1 384-1 | asym | K20 | 47 |
| SF-539 | | | | 261 | | | | 726 | 0 | 0 | 869 | 0 | 9,243 | 202 | R1 384-1 | asym | K21 | 21 |
| SK-MEL-28 | | | | 262 | | | | 148 | 0 | 0 | 1,157 | 273 | 23,788 | 634 | R1 384-1 | asym | K22 | 48 |
| K-562 | | | | 263 | | | | 313 | 683 | 0 | 763 | 597 | 4,477 | 300 | R1 384-1 | asym | K23 | 23 |
| UACC-257 | | | | 264 | | | | 245 | 0 | 0 | 26 | 0 | 2,255 | 330 | R1 384-1 | asym | K24 | 50 |
| M14 | | | | 265 | | | | 90 | 242 | 0 | 0 | 214 | 129,614 | 1,060 | R1 384-1 | asym | L01 | 51 |
| MCF7 | | | | 267 | | | | 204 | 0 | 0 | 16,366 | 0 | 432,526 | 111 | R1 384-1 | asym | L03 | 52 |
| MDA-MB-435 | | | | 269 | | | | 90 | 0 | 0 | 1,131 | 0 | 14,733 | 0 | R1 384-1 | asym | L05 | 56 |
| HT279 | | | | 270 | | | | 0 | 323 | 0 | 0 | 0 | 19,273 | 86 | R1 384-1 | asym | L06 | 185 |
| MDA-N | | | | 271 | | | | 12 | 176 | 0 | 89 | 0 | 9,135 | 317 | R1 384-1 | asym | L07 | 57 |
| Y79 poly A+ | | | | 273 | | | | 463 | 107 | 0 | 0 | 20 | 69,877 | 0 | R1 384-1 | asym | L09 | 61 |
| KHOS poly A+ | | | | 289 | | | | 0 | 0 | 0 | 0 | 0 | 75,507 | 0 | R1 384-1 | asym | M01 | 63 |
| HTB36 24h TPA | | | | 300 | | | | 5 | 50 | 0 | 0 | 86 | 34,541 | 0 | R1 384-1 | asym | M12 | 82 |
| RNA 6/23 | | | | | | | | | | | | | | | | | | |
| HELA-EXP-031899 | | | | 313 | | | | 0 | 0 | 0 | 110 | 0 | 55,070 | 45 | R1 384-1 | asym | N01 | 156 |
| HTB36 0h RNA | | | | 322 | | | | 0 | 20 | 0 | 0 | 0 | 49,195 | 54 | R1 384-1 | asym | N10 | 72 |
| HT347 | | | | 323 | | | | 0 | 282 | 0 | 521 | 0 | 46,274 | 127 | R1 384-1 | asym | N11 | 200 |
| 458 medullo RNA | | | | 324 | | | | 0 | 0 | 0 | 0 | 0 | 26,987 | 60 | R1 384-1 | asym | N12 | 74 |
| NCI-H226 | | | | 336 | | | | 214 | 352 | 0 | 0 | 257 | 2,962 | 0 | R1 384-1 | asym | N24 | 4 |
| HOP-62 | | | | 337 | | | | 219 | 0 | 0 | 0 | 0 | 11,386 | 157 | R1 384-1 | asym | O01 | 9 |
| MDA-MB-231 | | | | 338 | | | | 0 | 0 | 0 | 577 | 277 | 51,134 | 901 | R1 384-1 | asym | O02 | 55 |
| U251 | | | | 339 | | | | 0 | 237 | 0 | 4,549 | 515 | 55,589 | 372 | R1 384-1 | asym | O03 | 18 |
| PT cells poly A+ | | | | 340 | | | | 130 | 106 | 0 | 0 | 261 | 9,973 | 0 | R1 384-1 | asym | O04 | 60 |
| PC-3 | | | | 341 | | | | 0 | 284 | 0 | 0 | 1,140 | 65,936 | 1,366 | R1 384-1 | asym | O05 | 29 |
| HCC-2998 | | | | 343 | | | | 803 | 0 | 0 | 375 | 639 | 118,307 | 958 | R1 384-1 | asym | O07 | 30 |
| SW-620 | | | | 345 | | | | 0 | 0 | 79 | 22,607 | 0 | 467,592 | 1,300 | | | | |
| SW480-7 | | | | | | | | | | | | | | | R1 384-1 | asym | O09 | 32 |
| HT192 | | | | 346 | | | | 0 | 0 | 0 | 0 | 0 | 74,830 | 83 | R1 384-1 | asym | O10 | 179 |
| COLO 205 | | | | 347 | | | | 621 | 6 | 0 | 0 | 0 | 10,126 | 0 | R1 384-1 | asym | O11 | 33 |
| HT218 | | | | 348 | | | | 0 | 0 | 0 | 0 | 0 | 20,377 | 0 | R1 384-1 | asym | O12 | 182 |
| KM-12 | | | | 349 | | | | 860 | 259 | 0 | 623 | 0 | 21,590 | 0 | R1 384-1 | asym | O13 | 35 |
| HT151 | | | | 350 | | | | 0 | 411 | 0 | 347 | 0 | 60,081 | 414 | R1 384-1 | asym | O14 | 164 |
| A498 | | | | 351 | | | | 0 | 428 | 0 | 0 | 378 | 9,226 | 0 | R1 384-1 | asym | O15 | 38 |
| HT393 | | | | 352 | | | | 0 | 0 | 0 | 694 | 0 | 57,432 | 0 | R1 384-1 | asym | O16 | 215 |
| RXF 393 | | | | 353 | | | | 0 | 0 | 0 | 0 | 0 | 6,338 | 82 | R1 384-1 | asym | O17 | 40 |
| TK-10 | | | | 355 | | | | 191 | 0 | 0 | 2,314 | 0 | 14,179 | 0 | R1 384-1 | asym | O19 | 43 |
| Malme-3M | | | | 357 | | | | 0 | 0 | 0 | 1,531 | 885 | 10,560 | 174 | R1 384-1 | asym | O21 | 45 |
| Hs 578T | | | | 359 | | | | 212 | 0 | 0 | 0 | 9 | 2,568 | 0 | R1 384-1 | asym | O23 | 54 |
| HT213 | | | 50 | | | | | 886 | 0 | 0 | 0 | 0 | 48,907 | 0 | R1 384-1 | asym | C02 | 180 |
| HT288 | | | 52 | | | | | 0 | 224 | 0 | 693 | 0 | 74,176 | 18 | R1 384-1 | asym | C04 | 187 |
| HT139 | | | 54 | | | | | 0 | 0 | 0 | 631 | 0 | 67,320 | 102 | R1 384-1 | asym | C06 | 158 |
| HT155 | | | 56 | | | | | 0 | 0 | 0 | 30 | 0 | 61,739 | 0 | R1 384-1 | asym | C08 | 166 |
| HT163 | | | 58 | | | | | 48 | 0 | 0 | 0 | 345 | 80,453 | 275 | R1 384-1 | asym | C10 | 171 |
| HT170 | | | 60 | | | | | 0 | 184 | 0 | 773 | 0 | 63,967 | 0 | R1 384-1 | asym | C12 | 173 |
| HT172 | | | 62 | | | | | 0 | 0 | 0 | 0 | 0 | 48,537 | 0 | R1 384-1 | asym | C14 | 174 |
| HT138 | | | 63 | | | | | 10 | 1 | 0 | 741 | 0 | 46,416 | 156 | R1 384-1 | asym | C15 | 157 |
| HT178 | | | 64 | | | | | 0 | 14 | 0 | 301 | 0 | 57,882 | 330 | R1 384-1 | asym | C16 | 175 |
| HT154 | | | 65 | | | | | 0 | 166 | 0 | 0 | 0 | 69,411 | 89 | R1 384-1 | asym | C17 | 165 |
| HT180 | | | 66 | | | | | 47 | 57 | 0 | 0 | 0 | 53,278 | 214 | R1 384-1 | asym | C18 | 176 |
| HT169 | | | 67 | | | | | 0 | 0 | 0 | 0 | 0 | 23,349 | 0 | R1 384-1 | asym | C19 | 172 |
| HT189 | | | 68 | | | | | 182 | 0 | 0 | 0 | 0 | 63,765 | 243 | R1 384-1 | asym | C20 | 177 |
| HT143 | | | 69 | | | | | 0 | 0 | 0 | 86 | 0 | 43,069 | 129 | R1 384-1 | asym | C21 | 160 |
| HT190 | | | 70 | | | | | 0 | 0 | 0 | 10 | 0 | 51,091 | 22 | R1 384-1 | asym | C22 | 178 |
| HT145 | | | 71 | | | | | 372 | 0 | 0 | 0 | 0 | 58,721 | 0 | R1 384-1 | asym | C23 | 161 |
| HT227 | | | 72 | | | | | 101 | 0 | 0 | 173 | 29 | 33,093 | 0 | R1 384-1 | asym | C24 | 184 |
| HT302 | | | 73 | | | | | 56 | 207 | 0 | 0 | 0 | 69,267 | 275 | R1 384-1 | asym | D01 | 188 |
| HT314 | | | 74 | | | | | 0 | 20 | 0 | 1,141 | 0 | 60,939 | 287 | R1 384-1 | asym | D02 | 193 |
| HT317 | | | 76 | | | | | 0 | 0 | 0 | 0 | 0 | 51,326 | 484 | R1 384-1 | asym | D04 | 194 |
| Medulloblastoma | | | 77 | | | | | 0 | 74 | 0 | 774 | 0 | 76,589 | 60 | R1 384-1 | asym | D05 | 83 |
| #425 11/8 | | | | | | | | | | | | | | | | | | |
| HT323 | | | 78 | | | | | 0 | 0 | 0 | 0 | 0 | 111,765 | 339 | R1 384-1 | asym | D06 | 195 |
| HT327 | | | 80 | | | | | 0 | 251 | 0 | 691 | 432 | 62,791 | 338 | R1 384-1 | asym | D08 | 196 |
| HT335 | | | 82 | | | | | 0 | 25 | 0 | 176 | 0 | 69,031 | 0 | R1 384-1 | asym | D10 | 198 |
| HT146 | | | 85 | | | | | 227 | 0 | 0 | 0 | 405 | 7,160 | 0 | R1 384-1 | asym | D13 | 162 |
| HT348 | | | 87 | | | | | 359 | 0 | 0 | 0 | 0 | 69,239 | 1,165 | R1 384-1 | asym | D15 | 201 |
| HT311 | | | 170 | | | | | 0 | 75 | 0 | 0 | 0 | 31,571 | 246 | R1 384-1 | asym | H02 | 191 |
| HT398 | | | 185 | | | | | 3,484 | 281 | 0 | 0 | 259 | 5,903 | 31 | R1 384-1 | asym | H17 | 218 |
| HT140 | | | 187 | | | | | 662 | 0 | 0 | 0 | 0 | 1,081 | 0 | R1 384-1 | asym | H19 | 159 |
| HT281 | | | 189 | | | | | 549 | 0 | 0 | 0 | 231 | 5,431 | 0 | R1 384-1 | asym | H21 | 186 |
| HT372 | | | 191 | | | | | 236 | 0 | 0 | 0 | 0 | 11,267 | 0 | R1 384-1 | asym | H23 | 206 |
| TCGP | | | 207 | | | | | 0 | 0 | 0 | 0 | 265 | 9,599 | 16 | R1 384-1 | asym | I15 | 76 |
| HT160 | | | 216 | | | | | 0 | 0 | 0 | 0 | 0 | 2,822 | 0 | R1 384-1 | asym | I24 | 169 |
| HT307 | | | 217 | | | | | 144 | 149 | 0 | 0 | 0 | 27,812 | 84 | R1 384-1 | asym | J01 | 189 |
| HT369 | | | 224 | | | | | 0 | 103 | 0 | 0 | 0 | 45,642 | 0 | R1 384-1 | asym | J08 | 203 |
| HT370 | | | 226 | | | | | 0 | 130 | 0 | 0 | 0 | 33,703 | 22 | R1 384-1 | asym | J10 | 204 |
| HT371 | | | 228 | | | | | 0 | 58 | 0 | 576 | 0 | 46,710 | 453 | R1 384-1 | asym | J12 | 205 |
| HT377 | | | 230 | | | | | 0 | 132 | 0 | 0 | 0 | 35,666 | 354 | R1 384-1 | asym | J14 | 208 |
| HT382 | | | 236 | | | | | 101 | 328 | 0 | 0 | 0 | 28,197 | 45 | R1 384-1 | asym | J20 | 210 |
| neruoblastoma RNA | | | 281 | | | | | 1,184 | 396 | 0 | 2,805 | 0 | 0 | 0 | R1 384-1 | asym | L17 | 84 |
| HT334 | | | 299 | | | | | 0 | 0 | 0 | 0 | 0 | 111,643 | 377 | R1 384-1 | asym | M11 | 197 |
| HT338 | | | 301 | | | | | 0 | 56 | 0 | 0 | 0 | 28,893 | 0 | R1 384-1 | asym | M13 | 199 |
| HT392 | | | 315 | | | | | 460 | 0 | 0 | 0 | 0 | 19,458 | 155 | R1 384-1 | asym | N03 | 213 |
| HT394 | | | 317 | | | | | 0 | 0 | 0 | 46 | 62 | 37,089 | 235 | R1 384-1 | asym | N05 | 216 |
| HT312 | | | 319 | | | | | 181 | 36 | 0 | 0 | 245 | 28,897 | 0 | R1 384-1 | asym | N07 | 192 |
| HT162 | | | 325 | | | | | 0 | 0 | 0 | 0 | 0 | 17,011 | 0 | R1 384-1 | asym | N13 | 170 |
| HT395 | | | 358 | | | | | 233 | 0 | 0 | 0 | 0 | 7,637 | 39 | R1 384-1 | asym | O22 | 217 |
| HT157 | | | 360 | | | | | 0 | 383 | 0 | 0 | 0 | 13,226 | 0 | R1 384-1 | asym | O24 | 167 |
| 458 medullo RNA | | | | | | | | 138 | 154 | 0 | 163 | 0 | 90,329 | 690 | R1 384-1 | sym | B21 | 282 |
| CRL 1572 | | | | | | | | 0 | 0 | 0 | 248 | 0 | 56,547 | 0 | R1 384-1 | asym | D03 | 80 |
| Mar. 17, 1989 | | | | | | | | | | | | | | | | | | |
| h fibroblasts | | | | | | | | 423 | 390 | 0 | 0 | 102 | 12,530 | 184 | R1 384-1 | asym | L21 | 132 |
| Mar. 31, 1992 #12 | | | | | | | | | | | | | | | | | | |
| h keratinocytes | | | | | | | | 64 | 0 | 0 | 0 | 419 | 15,538 | 0 | R1 384-1 | asym | J16 | 133 |
| Feb. 25, 1992 #10 | | | | | | | | | | | | | | | | | | |
| HT308 | | | | | | | | 899 | 0 | 0 | 0 | 0 | 54,950 | 21 | R1 384-1 | asym | D23 | 190 |
| HT368 | | | | | | | | 0 | 31 | 0 | 0 | 80 | 66,952 | 0 | R1 384-1 | asym | D17 | 202 |
| HT378 | | | | | | | | 0 | 0 | 0 | 40 | 0 | 56,777 | 707 | R1 384-1 | asym | D19 | 209 |
| HT385 | | | | | | | | 0 | 0 | 0 | 846 | 0 | 33,000 | 0 | R1 384-1 | asym | D21 | 212 |

| Tissue_type | CC | DD | MC | SR | ANG | PRO | Mets | Prostate | Neuro | Heme | Serum | Mitosis | Cell Cycle | DNA Dam | Ken Lip | Angio | NCI-Ras | Hypoxia | PYK2 | “Diabetes” |
|---|---|---|---|---|---|---|---|---|---|---|---|---|---|---|---|---|---|---|---|---|
| TCGP | | | | | | | | | | | | | 26 | | | | | | | |
| h keratinocytes | | | | | | | | | | | | | | | | | | | | |
| Feb 25, 1992 #10 | | | | | | | | | | | | | | | | | | | | |
| h adult SMC | | | | | | | | | | | | | | | | | | | | |
| Oct. 21, 1992 #17 | | | | | | | | | | | | | | | | | | | | |
| h fibroblasts | | | | | | | | | | | | | | | | | | | | |
| Mar. 31, 1992 #12 | | | | | | | | | | | | | | | | | | | | |
| HOP-92 | | | | | | | | | | | | | | | | | NL | | | |
| OVCAR-8 | | | | | | | | | | | | | | | | | | | | |
| EKVX | | | | | | | | | | | | | | | | | | | | |
| IGROV1 | | | | | | | | | | | | | | | | | NL | | | |
| NCI-H23 | | | | | | | | | | | | | | | | | | | | |
| SK-OV-3 | | | | | | | | | | | | | | | | | | | | |
| NCI-H226 | | | | | | | | | | | | | | | | | | | | |
| SNB-19 | | | | | | | | | 104 | | | | | | | | NL | | | |
| NCI-H322M | | | | | | | | | | | | | | | | | NL | | | |
| SNB-75 | | | | | | | | | 106 | | | | | | | | NL | | | |
| NCI-H460 | | | | | | | | | | | | | | | | | KRas61 | | | |
| U251 | | | | | | | | | 108 | | | | | | | | NL | | | |
| NCI-H522 | | | | | | | | | | | | | | | | | | | | |
| SF-268 | | | | | | | | | 110 | | | | | | | | | | | |
| A549/ATCC | | | | | | | | | | | | | | | | | | | | |
| SF-295 | | | | | | | | | 112 | | | | | | | | NL | | | |
| HOP-62 | | | | | | | | | | | | | | | | | | | | |
| SF-539 | | | | | | | | | | | | | | | | | NL | | | |
| OVCAR-3 | | | | | | | | | 114 | | | | | | | | | | | |
| CCRF-CEM | | | | | | | | | | | | | | | | | | | | |
| OVCAR-4 | | | | | | | | | | | | | | | | | NL | | | |
| K-562 | | | | | | | | | | 118 | | | | | | | | | | |
| OVCAR-5 | | | | | | | | | | | | | | | | | KRas12 | | | |
| MOLT-4 | | | | | | | | | | 120 | | | | | | | | | | |
| HL-60 | | | | | | | | | | 121 | | | | | | | | | | |
| SN12C | | | | | | | | | | | | | | | | | | | | |
| RPMI 8226 | | | | | | | | | | 123 | | | | | | | KRas12 | | | |
| A498 | | | | | | | | | | | | | | | | | | | | |
| SR | | | | | | | | | | 125 | | | | | | | NRas12 | | | |
| Caki-1 | | | | | | | | | | | | | | | | | NL | | | |
| DU-145 | | | | | | | | 127 | | | | | | | | | NL | | | |
| RXF 393 | | | | | | | | | | | | | | | | | | | | |
| PC-3 | | | | | | | | 129 | | | | | | | | | | | | |
| ACHN | | | | | | | | | | | | | | | | | | | | |
| HCC-2998 | | | | | | | | | | | | | | | | | | | | |
| 786-0 | | | | | | | | | | | | | | | | | NL | | | |
| HCT 116 | | | | | | | | | | | | | | | | | | | | |
| TK-10 | | | | | | | | | | | | | | | | | | | | |
| SW480-8 | | | | | | | | | 136 | | | | | | | | KRas12 | | | |
| LOX IMVI | | | | | | | | | | | | | | | | | | | | |
| COLO 205 | | | | | | | | | | | | | | | | | NL | | | |
| Malme-3M | | | | | | | | | | | | | | | | | | | | |
| HCT-15 | | | | | | | | | | | | | | | | | KRas13 | | | |
| SK-MEL-2 | | | | | | | | | | | | | | | | | NRas61 | | | |
| KM-12 | | | | | | | | | | | | | | | | | NL | | | |
| SK-MEL-5 | | | | | | | | | | | | | | | | | | | | |
| UO-31 | | | | | | | | | | | | | | | | | | | | |
| SK-MEL-28 | | | | | | | | | | | | | | | | | NL | | | |
| UACC-62 | | | | | | | | | | | | | | | | | | | | |
| UACC-257 | | | | | | | | | | | | | | | | | NL | | | |
| M14 | | | | | | | | | | | | | | | | | | | | |
| MCF-7-1 | | | | | | | | | | | | | | | | | NL | | | |
| MCF7 | | | | | | | | | | | | | | | | | | | | |
| Hs 578T | | | | | | | | | | | | | | | | | | | | |
| MDA-MB-231 | | | | | | | | | | | | | | | | | KRas13 | | | |
| MDA-MB-435 | | | | | | | | | | | | | | | | | NL | | | |
| MDA-N | | | | | | | | | | | | | | | | | NL | | | |
| T-47D | | | | | | | | | | | | | | | | | NL | | | |
| ADR-RES-2 | | | | | | | | | | | | | | | | | | | | |
| lymph node-h | | | | | | | | | | 2 | | | | | | | | | | |
| bone marrow-h | | | | | | | | | | 3 | | | | | | | | | | |
| mammary gland-h | | | | | | | | | | | | | | | | | | | | |
| brain-h | | | | | | | | | 5 | | | | | | | | | | | |
| pancreas-h | | | | | | | | | | | | | | | | | | | | 6 |
| cerebellum-h | | | | | | | | | 7 | | | | | | | | | | | |
| pituitary gland-h | | | | | | | | | 8 | | | | | | | | | | | |
| fetal brain-h | | | | | | | | | 9 | | | | | | | | | | | |
| placenta-h | | | | | | | | | | | | | | | | 10 | | | | |
| fetal kidney-h | | | | | | | | | | | | | | | | 11 | | | | |
| prostate, h | | | | | | | | 12 | | | | | | | | | | | | |
| fetal liver-h | | | | | | | | | | | | | | | | 13 | | | | |
| Salivary gl.-h | | | | | | | | | | | | | | | | | | | | |
| fetal lung-h | | | | | | | | | | | | | | | | 15 | | | | |
| Skeletal muscle-h | | | | | | | | | | | | | | | | | | | | 16 |
| heart-h | | | | | | | | | | | | | | | | | | | | |
| small intestine-h | | | | | | | | | | | | | | | | | | | | |
| kidney-h | | | | | | | | | | | | | | | | | | | | |
| spinal cord-h | | | | | | | | | 20 | | | | | | | | | | | |
| liver-h | | | | | | | | | | | | | | | | | | | | |
| Spleen-h | | | | | | | | | | 22 | | | | | | | | | | |
| lung-h | | | | | | | | | | | | | | | | | | | | |
| stomach-h | | | | | | | | | | | | | | | | | | | | |
| testis-h | | | | | | | | | | | | | 25 | | | | | | | |
| thymus-h | | | | | | | | | | 27 | | | | | | | | | | |
| HPAEC | | | | | | | | | | | | | | | | 28 | | | | |
| thyroid gland-h | | | | | | | | | | | | | | | | | | | | |
| RPTEC | | | | | | | | | | | | | | | | 30 | | | | |
| trachea-h | | | | | | | | | | | | | | | | | | | | |
| HMEC | | | | | | | | | | | | | | | | | | | | |
| uterus-h | | | | | | | | | | | | | | | | | | | | |
| HCAEC | | | | | | | | | | | | | | | | 34 | | | | |
| pancreas-h | | | | | | | | | | | | | | | | | | | | 35 |
| lymph node-h | | | | | | | | | | 36 | | | | | | | | | | |
| Skeletal muscle-h | | | | | | | | | | | | | | | | | | | | 37 |
| fetal liver-h | | | | | | | | | | | | | | | | 38 | | | | |
| Heart-h | | | | | | | | | | | | | | | | | | | | |
| thymus, h | | | | | | | | | | 40 | | | | | | | | | | |
| Duodenum-h | | | | | | | | | | | | | | | | | | | | |
| fetal brain-h | | | | | | | | | 42 | | | | | | | | | | | |
| Salivary gl.-h | | | | | | | | | | | | | | | | | | | | |
| testis-h | | | | | | | | | | | | | 44 | | | | | | | |
| fetal kidney-h | | | | | | | | | | | | | | | | 49 | | | | |
| fetal liver-h | | | | | | | | | | | | | | | | 51 | | | | |
| fetal lung-h | | | | | | | | | | | | | | | | 53 | | | | |
| Heart-h | | | | | | | | | | | | | | | | | | | | |
| kidney-h | | | | | | | | | | | | | | | | | | | | |
| lung-h | | | | | | | | | | | | | | | | | | | | |
| lymph node-h | | | | | | | | | | 61 | | | | | | | | | | |
| WI-38 72h | | | | | | | | | | | | | | | | | | | | |
| thymus-h | | | | | | | | | | 193 | | | | | | | | | | |
| HEPM 3d TGFB1 | | | | | | | | | | | | | | | | | | | | |
| detergent + DNase | | | | | | | | | | | | | | | | | | | | |
| salivary gl.-h | | | | | | | | | | | | | | | | | | | | |
| testis-h | | | | | | | | | | | | | 199 | | | | | | | |
| Pancreas-h | | | | | | | | | | | | | | | | | | | | 201 |
| Skeletal muscle-h | | | | | | | | | | | | | | | | | | | | 203 |
| Duodenum-h | | | | | | | | | | | | | | | | | | | | |
| HMEC | | | | | | | | | | | | | | | | | | | | |
| Fetal brain-h | | | | | | | | | 210 | | | | | | | | | | | |
| HCAEC | | | | | | | | | | | | | | | | 211 | | | | |
| placenta-h | | | | | | | | | | | | | | | | 212 | | | | |
| fetal liver-h | | | | | | | | | | | | | | | | 213 | | | | |
| stomach-h | | | | | | | | | | | | | | | | | | | | |
| heart-h | | | | | | | | | | | | | | | | | | | | |
| bladder-h | | | | | | | | | | | | | | | | | | | | |
| HT372-normal | | | | | | | | | | | | | | | | | | | | |
| HT382-normal | | | | | | | | | | | | | | | | | | | | |
| HT392-normal | | | | | | | | | | | | | | | | | | | | |
| HPAEC | | | | | | | | | | | | | | | | 275 | | | | |
| ADR-RES-1 | | | | | | | | | | | | | | | | | | | | |
| bone marrow-h | | | | | | | | | | 279 | | | | | | | | | | |
| skeletal muscle-h | | | | | | | | | | | | | | | | | | | | |
| small intestine-h | | | | | | | | | | | | | | | | | | | | 290 |
| spinal cord-h | | | | | | | | | 294 | | | | | | | | | | | |
| Spleen-h | | | | | | | | | | | | | | | | | | | | |
| liver-h | | | | | | | | | | | | | | | | | | | | |
| testis-h | | | | | | | | | | | | 298 | | | | | | | | |
| bladder-h | | | | | | | | | | | | | | | | | | | | |
| mammary gland-h | | | | | | | | | | | | | | | | | | | | |
| Pancreas-h | | | | | | | | | | | | | | | | | | | | 305 |
| pituitary gland-h | | | | | | | | | | | | | | | | | | | | |
| prostate, h | | | | | | | | 309 | | | | | | | | | | | | |
| salivary gl-h | | | | | | | | | | | | | | | | | | | | |
| SA-OS (Mundy) | | | | | | | | | | | | | | | | | | | | |
| poly A+ | | | | | | | | | | | | | | | | | | | | |
| thyroid gland-h | | | | | | | | | | | | | | | | | | | | |
| trachea-h | | | | | | | | | | | | | | | | | | | | |
| uterus-h | | | | | | | | | | | | | | | | | | | | |
| HEPM 3d untreated | | | | | | | | | | | | | | | | | | | | |
| HT149-normal | | | | | | | | | | | | | | | | | | | | |
| thymus, h | | | | | | | | | | 326 | | | | | | | | | | |
| HT398-normal | | | | | | | | | | | | | | | | | | | | |
| Fetal brain-h | | | | | | | | | 328 | | | | | | | | | | | |
| h adult SMC | | | | | | | | | | | | | | | | | | | | |
| Oct. 21, 1992 #17 | | | | | | | | | | | | | | | | | | | | |
| lymph node-h | | | | | | | | | | | | | | | | | | | | |
| RPTEC | | | | | | | | | | | | | | | | | | | | |
| brain-h | | | | | | | | | 342 | | | | | | | 334 | | | | |
| cerebellum-h | | | | | | | | | 344 | | | | | | | | | | | |
| HT157-normal | | | | | | | | | | | | | | | | | | | | |
| HT213-normal | | | | | | | | | | | | | | | | | | | | |
| HT218-normal | | | | | | | | | | | | | | | | | | | | |
| HeLa-1 | CC-26 | | MC-31 | SR-21 | | | | | | | 58 | 58 | 58 | 58 | | | | | | |
| HeLa-2 | | | | SR-22 | | | | | | | 138 | | | | | | | | | |
| HeLa-3 | CC-27 | | | | | | | | | | | | 60 | | | | | | | |
| HeLa-4 | CC-28 | | | | | | | | | | | | 62 | 62 | | | | | | |
| HeLa-5 | CC-29 | | | | | | | | | | | | 64 | | | | | | | |
| HeLa-6 | | | | | | | | | | | | | | 66 | | | | | | |
| HeLa-7 | CC-30 | | MC-32 | | | | | | | | | 68 | 68 | | | | | | | |
| HeLa-8 | | | MC-33 | | | | | | | | | 70 | | | | | | | | |
| ADR-RES-3 | CC-16 | DD-28 | MC-28 | SR-19 | | | | | | | 65 | 65 | 65 | 65 | | | | | | |
| ADR-RES-4 | | | | SR-20 | | | | | | | 67 | | | | | | | | | |
| ADR-RES-5 | CC-17 | | | | | | | | | | | | 69 | | | | | | | |
| ADR-RES-6 | CC-18 | DD-29 | | | | | | | | | | | 71 | 71 | | | | | | |
| ADR-RES-7 | CC-19 | | | | | | | | | | | | 50 | | | | | | | |
| ADR-RES-8 | | DD-30 | | | | | | | | | | | | 52 | | | | | | |
| adrenal gland-h | CC-20 | | MC-29 | | | | | | | | | 54 | 54 | | | | | | | |
| adrenal gland-h | | | MC-30 | | | | | | | | | 56 | | | | | | | | |
| C33A-1 | | | MC-40 | SR-27 | | | | | | | 80 | 80 | 80 | 80 | | | | | | |
| C33A-2 | | | | SR-28 | | | | | | | 82 | | | | | | | | | |
| C33A-3 | | | | | | | | | | | | | 84 | | | | | | | |
| C33A-4 | | | | | | | | | | | | | 86 | 86 | | | | | | |
| C33A-5 | | | | | | | | | | | | | 88 | | | | | | | |
| C33A-6 | | | | | | | | | | | | | | 90 | | | | | | |
| C33A-7 | | | MC-41 | | | | | | | | | 92 | 92 | | | | | | | |
| C33A-8 | | | MC-42 | | | | | | | | | 94 | | | | | | | | |
| EKVX-1 | CC-06 | DD-04 | MC-04 | SR-03 | | | | | | | 122 | 122 | 122 | 122 | | | | | | |
| EKVX-2 | | | | SR-04 | | | | | | | 124 | | | | | | | | | |
| EKVX-3 | CC-08 | | | | | | | | | | | | 126 | | | | | | | |
| EKVX-4 | CC-07 | DD-05 | | | | | | | | | | | 128 | 128 | | | | | | 305 |
| EKVX-5 | CC-09 | | | | | | | | | | | | 17 | | | | | | | |
| EKVX-6 | | DD-06 | | | | | | | | | | | | 19 | | | | | | |
| EKVX-7 | CC-10 | | MC-05 | | | | | | | | | 21 | 21 | | | | | | | |
| EKVX-8 | | | MC-06 | | | | | | | | | 130 | | | | | | | | |
| H1299-1 | CC-31 | | MC-37 | SR-25 | | | | | | | 87 | 87 | 87 | 87 | | | | | | |
| H1299-2 | | | | SR-26 | | | | | | | 89 | | | | | | | | | |
| H1299-3 | CC-32 | | | | | | | | | | | | 91 | | | | | | | |
| H1299-4 | CC-33 | | | | | | | | | | | | 93 | 93 | | | | | | |
| H1299-5 | CC-34 | | | | | | | | | | | | 95 | | | | | | | |
| H1299-6 | | | | | | | | | | | | | | 74 | | | | | | |
| H1299-7 | CC-35 | | MC-38 | | | | | | | | | 76 | 76 | | | | | | | |
| H1299-8 | | | MC-39 | | | | | | | | | 78 | | | | | | | | |
| HT29-1 | | DD-10 | MC-10 | | | | | | | | 14 | 14 | 14 | 14 | | | | | | |
| HT29-2 | | | | SR-08 | | | | | | | 16 | | | | | | NL | | | |
| HT29-3 | | | | | | | | | | | | | 18 | | | | NL | | | |
| HT29-4 | | DD-11 | | | | | | | | | | | 20 | 20 | | | | | | |
| HT29-5 | | | | | | | | | | | | | 22 | | | | | | | |
| HT29-6 | | DD-12 | | | | | | | | | | | | 24 | | | | | | |
| HT29-7 | | | MC-11 | | | | | | | | | 25 | 25 | | | | | | | |
| HT29-8 | | | MC-12 | | | | | | | | | 27 | | | | | | | | |
| OVCAR-5-1 | | DD-22 | MC-22 | SR-15 | | | | | | | 36 | 36 | 36 | 36 | | | | | | |
| OVCAR-5-2 | | | | SR-16 | | | | | | | 38 | | | | | | | | | |
| OVCAR-5-3 | | | | | | | | | | | | | 40 | | | | | | | |
| OVCAR-5-4 | | DD-23 | | | | | | | | | | | 42 | 42 | | | | | | |
| OVCAR-5-5 | | | | | | | | | | | | | 171 | | | | | | | |
| OVCAR-5-6 | | DD-24 | | | | | | | | | | | | 46 | | | | | | |
| OVCAR-5-7 | | | MC-23 | | | | | | | | | 48 | 48 | | | | | | | |
| OVCAR-5-8 | | | MC-24 | | | | | | | | | 138 | | | | | | | | |
| SF-268-1 | | DD-16 | MC-16 | SR-11 | | | | | 120 | | 120 | 120 | 120 | 120 | | | NL | | | |
| SF-268-2 | | | | SR-12 | | | | | 121 | | 121 | | | | | | | | | |
| SF-268-3 | | | | | | | | | 123 | | | | 123 | | | | | | | |
| SF-268-4 | | DD-17 | | | | | | | 125 | | | | 125 | 125 | | | | | | |
| SF-268-5 | | | | | | | | | 127 | | | | 127 | | | | | | | |
| SF-268-6 | | DD-18 | | | | | | | 129 | | | | | 129 | | | | | | |
| SF-268-7 | | | MC-17 | | | | | | 131 | | | 131 | 131 | | | | | | | |
| SF-268-8 | | | MC-18 | | | | | | 133 | | | 133 | | | | | | | | |
| SW-620 | | | MC-34 | SR-23 | | | | | | | 72 | 72 | 72 | 72 | | | | | | |
| SW-620 | | | | SR-24 | | | | | | | 73 | | | | | | | | | |
| SW480-1 | | | | | | | | | | | | | 75 | | | | | | | |
| SW480-2 | | | | | | | | | | | | | 77 | 77 | | | | | | |
| SW480-3 | | | | | | | | | | | | | 79 | | | | | | | |
| SW480-4 | | | | | | | | | | | | | | 81 | | | | | | |
| SW480-5 | | | MC-35 | | | | | | | | | 83 | 83 | | | | | | | |
| SW480-6 | | | MC-36 | | | | | | | | | 85 | | | | | | | | |
| U2OS-1 | | | MC-43 | SR-29 | | | | | | | 96 | 96 | 96 | 96 | | | | | | |
| U2OS-2 | | | | SR-30 | | | | | | | 97 | | | | | | | | | |
| U2OS-3 | | | | | | | | | | | | | 99 | | | | | | | |
| U2OS-4 | | | | | | | | | | | | | 101 | 101 | | | | | | |
| U2OS-5 | | | | | | | | | | | | | 103 | | | | | | | |
| U2OS-6 | | | | | | | | | | | | | | 105 | | | | | | |
| U2OS-7 | | | MC-44 | | | | | | | | | 107 | 107 | | | | | | | |
| U2OS-8 | | | MC-45 | | | | | | | | | 109 | | | | | | | | |
| A549-1 | CC-01 | DD-01 | MC-01 | SR-01 | | | | | | | 1 | 1 | 1 | 1 | | | | | | |
| A549-2 | | | | SR-02 | | | | | | | 3 | | | | | | | | | |
| A549-3 | CC-02 | | | | | | | | | | | | 5 | | | | | | | |
| A549-4 | CC-03 | DD-02 | | | | | | | | | | | 7 | 7 | | | | | | |
| A549-5 | CC-04 | | | | | | | | | | | | 9 | | | | | | | |
| A549-6 | | DD-03 | | | | | | | | | | | | 11 | | | | | | |
| A549-7 | CC-05 | | MC-02 | | | | | | | | | 13 | 13 | | | | | | | |
| A549-8 | | | MC-03 | | | | | | | | | 15 | | | | | | | | |
| HCT-116-1 | | DD-07 | MC-07 | SR-05 | | | | | | | 23 | 23 | 23 | 23 | | | | | | |
| HCT-116-2 | | | | SR-06 | | | | | | | 132 | | | | | | | | | |
| HCT-116-3 | | | | | | | | | | | | | | | | | KRas13 | | | |
| HCT-116-4 | | DD-08 | | | | | | | | | | | 4 | 4 | | | | | | |
| HCT-116-5 | | | | | | | | | | | | | 6 | | | | | | | |
| HCT-116-6 | | DD-09 | | | | | | | | | | | | 8 | | | | | | |
| HCT-116-7 | | | MC-08 | | | | | | | | | 10 | 10 | | | | | | | |
| HCT-116-8 | | | MC-09 | | | | | | | | | 12 | | | | | | | | |
| Hs68-1 | | | MC-46 | SR-31 | | | | | | | 111 | 111 | 111 | 111 | | | | | | |
| Hs68-2 | | | | SR-32 | | | | | | | 113 | | | | | | | | | |
| Hs68-3 | | | | | | | | | | | | | 115 | | | | | | | |
| Hs68-4 | | | | | | | | | | | | | 117 | 117 | | | | | | |
| Hs68-6 | | | | | | | | | | | | | | 98 | | | | | | |
| Hs68-7 | | | MC-47 | | | | | | | | | 100 | 100 | | | | | | | |
| Hs68-8 | | | MC-48 | | | | | | | | | 102 | | | | | | | | |
| MCF-7-3 | CC-11 | DD-25 | MC-25 | SR-17 | | | | | | | 49 | 49 | 49 | 49 | | | | | | |
| MCF-7-4 | | | | SR-18 | | | | | | | 51 | | | | | | | | | |
| MCF-7-5 | CC-12 | | | | | | | | | | | | 53 | | | | | | | |
| MCF-7-6 | CC-13 | DD-26 | | | | | | | | | | | 55 | 55 | | | | | | |
| MCF-7-7 | CC-14 | | | | | | | | | | | | 57 | | | | | | | |
| MCF-7-8 | | DD-27 | | | | | | | | | | | | 59 | | | | | | |
| MCF-7/ADR-RES | CC-15 | | MC-26 | | | | | | | | | 61 | 61 | | | | | | | |
| MCF-7/ADR-RES | | | MC-27 | | | | | | | | | 63 | | | | | | | | |
| OVCAR-4-1 | | DD-19 | MC-19 | SR-13 | | | | | | | 45 | 45 | 45 | 45 | | | | | | |
| OVCAR-4-2 | | | | SR-14 | | | | | | | 47 | | | | | | | | | |
| OVCAR-4-3 | | | | | | | | | | | | | 26 | | | | | | | |
| OVCAR-4-4 | | DD-20 | | | | | | | | | | | 28 | 28 | | | | | | |
| OVCAR-4-5 | | | | | | | | | | | | | 30 | | | | | | | |
| OVCAR-4-6 | | DD-21 | | | | | | | | | | | | 32 | | | | | | |
| OVCAR-4-7 | | | MC-20 | | | | | | | | | 34 | 34 | | | | | | | |
| OVCAR-4-8 | | | MC-21 | | | | | | | | | 169 | | | | | | | | |
| SF539-1 | | DD-13 | MC-13 | SR-09 | | | | | 29 | | 29 | 29 | 29 | 29 | | | | | | |
| SF539-2 | | | | SR-10 | | | | | 31 | | 31 | | | | | | | | | |
| SF539-3 | | | | | | | | | 33 | | | | 33 | | | | | | | |
| SF539-4 | | DD-14 | | | | | | | 35 | | | | 35 | 35 | | | | | | |
| SF539-5 | | | | | | | | | 37 | | | | 37 | | | | | | | |
| SF539-6 | | DD-15 | | | | | | | 39 | | | | | 39 | | | | | | |
| SF539-7 | | | MC-14 | | | | | | 41 | | | 41 | 41 | | | | | | | |
| SF539-8 | | | MC-15 | | | | | | 43 | | | 43 | | | | | | | | |
| WI 38-1 | CC-21 | DD-31 | MC-49 | SR-33 | | | | | | | 104 | 104 | 104 | 104 | | | | | | |
| WI 38-2 | | | | SR-34 | | | | | | | 106 | | | | | | | | | |
| WI 38-3 | CC-22 | | | | | | | | | | | | 108 | | | | | | | |
| WI 38-4 | CC-23 | DD-32 | | | | | | | | | | | 110 | 110 | | | | | | |
| WI 38-5 | CC-24 | | | | | | | | | | | | 112 | | | | | | | |
| WI 38-6 | | DD-33 | | | | | | | | | | | | 114 | | | | | | |
| WI 38-7 | CC-25 | | MC-50 | | | | | | | | | 116 | 116 | | | | | | | |
| WI 38-8 | | | MC-51 | | | | | | | | | 118 | | | | | | | | |
| HELA-2h-031899 | | | | | | | | | | | | | 79 | | | | | | | |
| HELA-4h-031899 | | | | | | | | | | | | | 81 | | | | | | | |
| HELA-9h-031899 | | | | | | | | | | | | | 83 | | | | | | | |
| HELA-0h-031899 | | | | | | | | | | | | | 86 | | | | | | | |
| HELA-6h-031899 | | | | | | | | | | | | | 88 | | | | | | | |
| HELA-8h-031899 | | | | | | | | | | | | | 90 | | | | | | | |
| HELA-10h-031899 | | | | | | | | | | | | | 92 | | | | | | | |
| HELA-11h-031899 | | | | | | | | | | | | | 94 | | | | | | | |
| HELA-12h-031899 | | | | | | | | | | | | | 96 | | | | | | | |
| NCI-H322M | | | | | | | | | | | | | | | | | | | | |
| NCI-H460 | | | | | | | | | | | | | | | | | | | | |
| NCI-H522 | | | | | | | | | | | | | | | | | | | | |
| SNB-19 | | | | | | | | | 152 | | | | | | | | | | | |
| SNB-75 | | | | | | | | | | | | | | | | | | | | |
| SF-268 | | | | | | | | | 156 | | | | | | | | | | | |
| SF-295 | | | | | | | | | 158 | | | | | | | | | | | |
| CCRF-CEM | | | | | | | | | | 160 | | | | | | | | | | |
| DU-145 | | | | | | | | | 162 | | | | | | | | NL | | | |
| HCT 116 | | | | | | | | | | | | | | | | | | | | |
| Cacki-1 | | | | | | | | | | | | | | | | | | | | |
| 786-0 | | | | | | | | | | | | | | | | | NL | | | |
| T-47D | | | | | | | | | | | | | | | | | | | | |
| CRL1441 RNA 8/30 | | | | | | | | | | | | | | | | | | | | |
| 781T untreeated + | | | | | | | | | | | | | | | | | | | | |
| DNase | | | | | | | | | | | | | | | | | | | | |
| KB poly A+ | | | | | | | | | | | | | | | | | | | | |
| HOS poly A+ | | | | | | | | | | | | | | | | | | | | |
| ACHN | | | | | | | | | | | | | | | | | NL | | | |
| UACC-62 | | | | | | | | | | | | | | | | | NL | | | |
| MCF7 | | | | | | | | | | | | | | | | | NL | | | |
| UTOS (Mundy) | | | | | | | | | | | | | | | | | | | | |
| poly a+ | | | | | | | | | | | | | | | | | | | | |
| WISH (Collagen) | | | | | | | | | | | | | | | | | | | | |
| poly A+ | | | | | | | | | | | | | | | | | | | | |
| 458 medullo mRNA | | | | | | | | | | | | | | | | | | | | |
| CCL137 RNA | | | | | | | | | | | | | | | | | | | | |
| Mar. 21, 1988 | | | | | | | | | | | | | | | | | | | | |
| WI-38 72 h 0.5% | | | | | | | | | | | | | | | | | | | | |
| FBS, 24 h 10% FBS | | | | | | | | | | | | | | | | | | | | |
| CRL 1441 + TPA | | | | | | | | | | | | | | | | | | | | |
| (24h) 8/30 | | | | | | | | | | | | | | | | | | | | |
| HOP-92 | | | | | | | | | | | | | | | | | | | | |
| MOLT-4 | | | | | | | | | | 242 | | | | | | | NRas12- | | | |
| EKVX | | | | | | | | | | | | | | | | | NL | | | |
| HL-60 | | | | | | | | | | 244 | | | | | | | NRas61 | | | |
| NCI-H23 | | | | | | | | | | | | | | | | | NRas12 | | | |
| RPMI 8226 | | | | | | | | | | 246 | | | | | | | | | | |
| A549/ATCC | | | | | | | | | | | | | | | | | KRas12 | | | |
| SR | | | | | | | | | | 248 | | | | | | | | | | |
| OVCAR-3 | | | | | | | | | | | | | | | | | NL | | | |
| HCT-15 | | | | | | | | | | | | | | | | | | | | |
| OVCAR-4 | | | | | | | | | | | | | | | | | NL | | | |
| UO-31 | | | | | | | | | | | | | | | | | NL | | | |
| OVCAR-5 | | | | | | | | | | | | | | | | | | | | |
| SN12C | | | | | | | | | | | | | | | | | | | | |
| OVCAR-8 | | | | | | | | | | | | | | | | | NL | | | |
| LOX IMVI | | | | | | | | | | | | | | | | | NL | | | |
| IGROV1 | | | | | | | | | | | | | | | | | | | | |
| SK-MEL-2 | | | | | | | | | | | | | | | | | | | | |
| SK-OV-3 | | | | | | | | | | | | | | | | | NL | | | |
| SK-MEL-5 | | | | | | | | | | | | | | | | | NL | | | |
| SF-539 | | | | | | | | | | | | | | | | | | | | |
| SK-MEL-28 | | | | | | | | | | | | | | | | | | | | |
| K-562 | | | | | | | | | | 263 | | | | | | | | | | |
| UACC-257 | | | | | | | | | | | | | | | | | | | | |
| M14 | | | | | | | | | | | | | | | | | NL | | | |
| MCF-7-2 | | | | | | | | | | | | | | | | | | | | |
| MDA-MB-435 | | | | | | | | | | | | | | | | | | | | |
| HT279 | | | | | | | | | | | | | | | | | | | | |
| MDA-N | | | | | | | | | | | | | | | | | | | | |
| Y79 poly A+ | | | | | | | | | | | | | | | | | | | | |
| KHOS poly A+ | | | | | | | | | | | | | | | | | | | | |
| HTB36 24 h TPA | | | | | | | | | | | | | | | | | | | | |
| RNA 6/23 | | | | | | | | | | | | | | | | | | | | |
| HELA-EXP-031899 | | | | | | | | | | | | | 313 | | | | | | | |
| HTB36 0h RNA | | | | | | | | | | | | | | | | | | | | |
| HT347 | | | | | | | | | | | | | | | | | | | | |
| 458 medullo RNA | | | | | | | | | 324 | | | | | | | | | | | |
| NCI-H226 | | | | | | | | | | | | | | | | | NL | | | |
| HOP-62 | | | | | | | | | | | | | | | | | KRas12 | | | |
| MDA-MB-231 | | | | | | | | | | | | | | | | | | | | |
| U251 | | | | | | | | | | | | | | | | | | | | |
| PT cells poly A+ | | | | | | | | | | | | | | | | | | | | |
| PC-3 | | | | | | | | 341 | | | | | | | | | NL | | | |
| HCC-2998 | | | | | | | | | | | | | | | | | NL | | | |
| SW480-7 | | | | | | | | | | | | | | | | | | | | |
| HT192 | | | | | | | | | | | | | | | | | | | | |
| COLO 205 | | | | | | | | | | | | | | | | | | | | |
| HT218 | | | | | | | | | | | | | | | | | | | | |
| KM-12 | | | | | | | | | | | | | | | | | | | | |
| HT151 | | | | | | | | | | | | | | | | | | | | |
| A498 | | | | | | | | | | | | | | | | | | | | |
| HT393 | | | | | | | | | | | | | | | | | | | | |
| RXF 393 | | | | | | | | | | | | | | | | | NL | | | |
| TK-10 | | | | | | | | | | | | | | | | | NL | | | |
| Malme-3M | | | | | | | | | | | | | | | | | NL | | | |
| Hs 578T | | | | | | | | | | | | | | | | | HRas12 | | | |
| HT213 | | | | | | | | | | | | | | | | | | | | |
| HT288 | | | | | | | | | | | | | | | | | | | | |
| HT139 | | | | | | | | | | | | | | | | | | | | |
| HT155 | | | | | | | | | | | | | | | | | | | | |
| HT163 | | | | | | | | | | | | | | | | | | | | |
| HT170 | | | | | | | | | | | | | | | | | | | | |
| HT172 | | | | | | | | | | | | | | | | | | | | |
| HT138 | | | | | | | | | | | | | | | | | | | | |
| HT178 | | | | | | | | | | | | | | | | | | | | |
| HT154 | | | | | | | | | | | | | | | | | | | | |
| HT180 | | | | | | | | | | | | | | | | | | | | |
| HT169 | | | | | | | | | | | | | | | | | | | | |
| HT189 | | | | | | | | | | | | | | | | | | | | |
| HT143 | | | | | | | | | | | | | | | | | | | | |
| HT190 | | | | | | | | | | | | | | | | | | | | |
| HT145 | | | | | | | | | | | | | | | | | | | | |
| HT227 | | | | | | | | | | | | | | | | | | | | |
| HT302 | | | | | | | | | | | | | | | | | | | | |
| HT314 | | | | | | | | | | | | | | | | | | | | |
| HT317 | | | | | | | | | | | | | | | | | | | | |
| Medulloblastoma | | | | | | | | | | 77 | | | | | | | | | | |
| #425 11/8 | | | | | | | | | | | | | | | | | | | | |
| HT323 | | | | | | | | | | | | | | | | | | | | |
| HT327 | | | | | | | | | | | | | | | | | | | | |
| HT335 | | | | | | | | | | | | | | | | | | | | |
| HT146 | | | | | | | | | | | | | | | | | | | | |
| HT348 | | | | | | | | | | | | | | | | | | | | |
| HT311 | | | | | | | | | | | | | | | | | | | | |
| HT398 | | | | | | | | | | | | | | | | | | | | |
| HT140 | | | | | | | | | | | | | | | | | | | | |
| HT281 | | | | | | | | | | | | | | | | | | | | |
| HT372 | | | | | | | | | | | | | | | | | | | | |
| TCGP | | | | | | | | | | | | | | | | | | | | |
| HT160 | | | | | | | | | | | | | 207 | | | | | | | |
| HT307 | | | | | | | | | | | | | | | | | | | | |
| HT369 | | | | | | | | | | | | | | | | | | | | |
| HT370 | | | | | | | | | | | | | | | | | | | | |
| HT371 | | | | | | | | | | | | | | | | | | | | |
| HT377 | | | | | | | | | | | | | | | | | | | | |
| HT382 | | | | | | | | | | | | | | | | | | | | |
| neruoblastoma RNA | | | | | | | | | 281 | | | | | | | | | | | |
| HT334 | | | | | | | | | | | | | | | | | | | | |
| HT338 | | | | | | | | | | | | | | | | | | | | |
| HT392 | | | | | | | | | | | | | | | | | | | | |
| HT394 | | | | | | | | | | | | | | | | | | | | |
| HT312 | | | | | | | | | | | | | | | | | | | | |
| HT162 | | | | | | | | | | | | | | | | | | | | |
| HT395 | | | | | | | | | | | | | | | | | | | | |
| HT157 | | | | | | | | | | | | | | | | | | | | |
| 458 medullo RNA | | | | | | | | | | | | | | | | | | | | |
| CRL1572 | | | | | | | | | | | | | | | | | | | | |
| Mar. 17, 1989 | | | | | | | | | | | | | | | | | | | | |
| h fibroblasts | | | | | | | | | | | | | | | | | | | | |
| Mar. 31, 1992 #12 | | | | | | | | | | | | | | | | | | | | |
| h keratinocytes | | | | | | | | | | | | | | | | | | | | |
| Feb. 25, 1992 #10 | | | | | | | | | | | | | | | | | | | | |
| HT308 | | | | | | | | | | | | | | | | | | | | |
| HT368 | | | | | | | | | | | | | | | | | | | | |
| HT378 | | | | | | | | | | | | | | | | | | | | |
| HT385 | | | | | | | | | | | | | | | | | | | | |

| | Tumor | Normal | Tumor | Tumor | | | | SGK187 | SGK124 | SGK386 | SGK003 | SGK093 | SGK074 | SGK396 | | | | NCI/ |
|---|---|---|---|---|---|---|---|---|---|---|---|---|---|---|---|---|---|---|
| Tissue | sym | sym | 1o | cells | Normal | Endos | p53 | ID#NA_1 | ID#NA_9 | ID#NA_21 | ID#NA_22 | ID#NA_31 | ID#NA_32 | ID#NA_43 | Release | tag | pos | etc |
| TCGP | 26 | | | | | | | 212 | 0 | 0 | 2,366 | 0 | 58,451 | 2,077 | R1 384-1 | sym | B02 | 284 |
| n keratinocytes | 46 | | | | | | | 83 | 3,050 | 0 | 2,340 | 0 | 89,541 | 1,223 | R1 384-1 | sym | B22 | 330 |
| Feb. 25, 1992 #10 | | | | | | | | | | | | | | | | | | |
| h adult SMC | 47 | | | | | | | 0 | 0 | 0 | 1,713 | 374 | 73,933 | 291 | R1 384-1 | sym | B23 | 283 |
| Oct. 21, 1992 #17 | | | | | | | | | | | | | | | | | | |
| h fibroblasts | 48 | | | | | | | 0 | 0 | 0 | 423 | 152 | 62,710 | 1,235 | R1 384-1 | sym | B24 | 329 |
| Mar. 31, 1992 #12 | | | | | | | | | | | | | | | | | | |
| HOP-92 | 97 | | | | | | | 514 | 0 | 0 | 2,631 | 94 | 87,584 | 1,316 | R1 384-1 | sym | E01 | 224 |
| OVCAR-8 | 98 | | | | | | | 16 | 0 | 0 | 0 | 0 | 4,204 | 164 | R1 384-1 | sym | E02 | 236 |
| EKVX | 99 | | | | | | | 19 | 0 | 0 | 231 | 0 | 27,721 | 105 | R1 384-1 | sym | E03 | 225 |
| IGROV1 | 100 | | | | | | | 0 | 0 | 0 | 65 | 0 | 7,800 | 0 | R1 384-1 | sym | E04 | 237 |
| NCI-H23 | 101 | | | | | | | 0 | 0 | 0 | 0 | 0 | 26,620 | 70 | R1 384-1 | sym | E05 | 226 |
| SK-OV-3 | 102 | | | | | | | 260 | 0 | 0 | 1,474 | 0 | 65,008 | 1,516 | R1 384-1 | sym | E06 | 238 |
| NCI-H226 | 103 | | | | | | | 153 | 365 | 0 | 1,091 | 91 | 107,932 | 3,834 | R1 384-1 | sym | E07 | 227 |
| SNB-19 | 104 | | | | | | | 0 | 0 | 0 | 855 | 0 | 74,412 | 1,302 | R1 384-1 | sym | E08 | 239 |
| NCI-H322M | 105 | | | | | | | 243 | 77 | 0 | 422 | 226 | 35,551 | 84 | R1 384-1 | sym | E09 | 228 |
| SNB-75 | 106 | | | | | | | 138 | 0 | 0 | 0 | 0 | 98,053 | 1,797 | R1 384-1 | sym | E10 | 240 |
| NCI-H460 | 107 | | | | | | | 0 | 548 | 0 | 3,348 | 81 | 78,829 | 1,166 | R1 384-1 | sym | E11 | 229 |
| U251 | 108 | | | | | | | 0 | 554 | 0 | 0 | 0 | 84,310 | 677 | R1 384-1 | sym | E12 | 241 |
| NCI-H522 | 109 | | | | | | | 196 | 0 | 0 | 162 | 0 | 22,554 | 497 | R1 384-1 | sym | E13 | 230 |
| SF-268 | 110 | | | | | | | 0 | 771 | 0 | 0 | 593 | 49,287 | 1,115 | R1 384-1 | sym | E14 | 242 |
| A549/ATCC | 111 | | | | | | | 0 | 266 | 0 | 1,613 | 1,619 | 43,786 | 1,448 | R1 384-1 | sym | E15 | 231 |
| SF-295 | 112 | | | | | | | 0 | 0 | 0 | 1,333 | 0 | 7,106 | 0 | R1 384-1 | sym | E16 | 243 |
| HOP-62 | 113 | | | | | | | 231 | 343 | 0 | 529 | 1,746 | 21,968 | 670 | R1 384-1 | sym | E17 | 232 |
| SF-539 | 114 | | | | | | | 176 | 101 | 0 | 237 | 167 | 39,588 | 1,662 | R1 384-1 | sym | E18 | 244 |
| OVCAR-3 | 115 | | | | | | | 0 | 179 | 0 | 998 | 442 | 28,838 | 737 | R1 384-1 | sym | E19 | 233 |
| CCRF-CEM | 116 | | | | | | | 80 | 6,816 | 0 | 605 | 0 | 45,975 | 2,130 | R1 384-1 | sym | E20 | 245 |
| OVCAR-4 | 117 | | | | | | | 52 | 57 | 0 | 704 | 292 | 34,439 | 1,510 | R1 384-1 | sym | E21 | 234 |
| K-562 | 118 | | | | | | | 0 | 0 | 0 | 0 | 185 | 33,123 | 1,072 | R1 384-1 | sym | E22 | 246 |
| OVCAR-5 | 119 | | | | | | | 0 | 0 | 0 | 1,307 | 542 | 18,164 | 23 | R1 384-1 | sym | E23 | 235 |
| MOLT-4 | 120 | | | | | | | 0 | 0 | 0 | 118 | 133 | 35,133 | 410 | R1 384-1 | sym | E24 | 247 |
| HL-60 | 121 | | | | | | | 0 | 537 | 0 | 0 | 659 | 80,247 | 719 | R1 384-1 | sym | F01 | 248 |
| SN12C | 122 | | | | | | | 32 | 0 | 0 | 0 | 711 | 76,151 | 1,579 | R1 384-1 | sym | F02 | 260 |
| RPMI 8226 | 123 | | | | | | | 0 | 0 | 0 | 0 | 0 | 15,177 | 419 | R1 384-1 | sym | F03 | 249 |
| A498 | 124 | | | | | | | 0 | 904 | 0 | 430 | 3,358 | 122,082 | 962 | R1 384-1 | sym | F04 | 261 |
| SR | 125 | | | | | | | 80 | 0 | 0 | 0 | 553 | 97,140 | 1,884 | R1 384-1 | sym | F05 | 250 |
| Caki-1 | 126 | | | | | | | 0 | 0 | 0 | 1,099 | 6,386 | 88,518 | 1,978 | R1 384-1 | sym | F06 | 262 |
| OU-145 | 127 | | | | | | | 560 | 191 | 0 | 169 | 0 | 3,687 | 0 | R1 384-1 | sym | F07 | 251 |
| RXF 393 | 128 | | | | | | | 51 | 202 | 0 | 685 | 1,192 | 108,097 | 1,825 | R1 384-1 | sym | F08 | 263 |
| PC-3 | 129 | | | | | | | 0 | 0 | 0 | 0 | 55 | 27,208 | 99 | R1 384-1 | sym | F09 | 252 |
| ACHN | 130 | | | | | | | 0 | 0 | 0 | 1,248 | 0 | 21,998 | 0 | R1 384-1 | sym | F10 | 264 |
| HCC-2998 | 131 | | | | | | | 0 | 0 | 0 | 0 | 0 | 14,182 | 0 | R1 384-1 | sym | F11 | 253 |
| 786-0 | 132 | | | | | | | 0 | 443 | 0 | 500 | 302 | 74,251 | 689 | R1 384-1 | sym | F12 | 265 |
| HCT 116 | 133 | | | | | | | 489 | 149 | 0 | 397 | 0 | 2,376 | 0 | R1 384-1 | sym | F13 | 254 |
| TK-10 | 134 | | | | | | | 94 | 102 | 0 | 1,682 | 415 | 51,674 | 1,842 | R1 384-1 | sym | F14 | 266 |
| SW-620 | 135 | | | | | | | 503 | 25 | 148 | 21,381 | 90 | 479,097 | 853 | R1 384-1 | sym | F15 | 255 |
| LOX IMVI | 136 | | | | | | | 865 | 0 | 0 | 0 | 610 | 8,504 | 241 | R1 384-1 | sym | F16 | 267 |
| COLO 205 | 137 | | | | | | | 608 | 348 | 0 | 223 | 628 | 60,452 | 610 | R1 384-1 | sym | F17 | 256 |
| Malme-3M | 138 | | | | | | | 134 | 260 | 0 | 416 | 734 | 33,878 | 1,065 | R1 384-1 | sym | F18 | 268 |
| HCT-15 | 139 | | | | | | | 69 | 281 | 0 | 0 | 781 | 35,146 | 562 | R1 384-1 | sym | F19 | 257 |
| SK-MEL-2 | 140 | | | | | | | 720 | 219 | 0 | 1,523 | 340 | 21,933 | 648 | R1 384-1 | sym | F20 | 269 |
| KM-12 | 141 | | | | | | | 0 | 227 | 0 | 0 | 0 | 23,168 | 0 | R1 384-1 | sym | F21 | 258 |
| SK-MEL-5 | 142 | | | | | | | 346 | 1,100 | 0 | 1,862 | 319 | 20,650 | 825 | R1 384-1 | sym | F22 | 270 |
| UO-31 | 143 | | | | | | | 1,235 | 0 | 0 | 0 | 0 | 43,858 | 549 | R1 384-1 | sym | F23 | 259 |
| SK-MEL-28 | 144 | | | | | | | 0 | 121 | 0 | 362 | 151 | 2,283 | 158 | R1 384-1 | sym | F24 | 271 |
| UACC-62 | 145 | | | | | | | 0 | 74 | 0 | 473 | 1,045 | 106,158 | 1,533 | R1 384-1 | sym | G01 | 272 |
| UACC-257 | 147 | | | | | | | 1,027 | 131 | 0 | 1,889 | 0 | 132,416 | 2,031 | R1 384-1 | sym | G03 | 273 |
| M14 | 149 | | | | | | | 0 | 182 | 0 | 67 | 0 | 14,280 | 59 | R1 384-1 | sym | G05 | 274 |
| MCF7 | 151 | | | | | | | 66 | 0 | 106 | 17,887 | 0 | 476,093 | 791 | R1 384-1 | sym | G07 | 275 |
| MCF-7/ADR-RES | 153 | | | | | | | 0 | 0 | 0 | 972 | 484 | 87,261 | 1,487 | R1 384-1 | sym | G09 | 276 |
| Hs 578T | 155 | | | | | | | 0 | 0 | 0 | 1,717 | 0 | 125,561 | 611 | R1 384-1 | sym | G11 | 277 |
| MDA-MB-231 | 157 | | | | | | | 0 | 416 | 0 | 0 | 107 | 77,227 | 0 | R1 384-1 | sym | G13 | 278 |
| MDA-MB-435 | 159 | | | | | | | 391 | 359 | 0 | 1,576 | 0 | 41,654 | 1,257 | R1 384-1 | sym | G15 | 279 |
| MDA-N | 161 | | | | | | | 0 | 183 | 0 | 2,141 | 380 | 28,371 | 1,457 | R1 384-1 | sym | G17 | 280 |
| T-47D | 163 | | | | | | | 139 | 103 | 0 | 0 | 202 | 52,411 | 220 | R1 384-1 | sym | G19 | 281 |
| adrenal gland-h | | 1 | | | | | | 64 | 29 | 0 | 16,403 | 133 | 808,208 | 1,624 | R1 384-1 | sym | A01 | 285 |
| lymph node-h | | 2 | | | | | | 0 | 858 | 0 | 2,522 | 0 | 126,297 | 6,190 | R1 384-1 | sym | A02 | 297 |
| bone marrow-h | | 3 | | | | | | 0 | 793 | 0 | 157 | 435 | 127,325 | 3,459 | R1 384-1 | sym | A03 | 286 |
| mammary gland-h | | 4 | | | | | | 0 | 0 | 0 | 0 | 221 | 60,842 | 482 | R1 384-1 | sym | A04 | 298 |
| brain-h | | 5 | | | | | | 0 | 309 | 0 | 206 | 884 | 109,916 | 4,188 | R1 384-1 | sym | A05 | 287 |
| pancreas-h | | 6 | | | | | | 0 | 400 | 0 | 1,126 | 0 | 103,318 | 2,814 | R1 384-1 | sym | A06 | 299 |
| cerebellum-h | | 7 | | | | | | 0 | 87 | 0 | 9,410 | 578 | 167,028 | 9,694 | R1 384-1 | sym | A07 | 288 |
| pituitary gland-h | | 8 | | | | | | 83 | 1,273 | 0 | 1,820 | 194 | 106,893 | 5,097 | R1 384-1 | sym | A08 | 300 |
| fetal brain-h | | 9 | | | | | | 359 | 1,004 | 0 | 4,928 | 862 | 121,604 | 8,651 | R1 384-1 | sym | A09 | 289 |
| placenta-h | | 10 | | | | | | 0 | 0 | 0 | 6,212 | 1,310 | 115,612 | 2,852 | R1 384-1 | sym | A10 | 301 |
| fetal kidney-h | | 11 | | | | | | 0 | 0 | 0 | 4,629 | 28,058 | 192,142 | 4,075 | R1 384-1 | sym | A11 | 290 |
| prostate, h | | 12 | | | | | | 0 | 290 | 0 | 851 | 0 | 18,517 | 0 | R1 384-1 | sym | A12 | 302 |
| fetal liver-h | | 13 | | | | | | 5,768 | 69 | 0 | 2,652 | 7,321 | 128,455 | 2,039 | R1 384-1 | sym | A13 | 291 |
| salivary gl.-h | | 14 | | | | | | 0 | 0 | 0 | 1,389 | 0 | 92,749 | 1,033 | R1 384-1 | sym | A14 | 303 |
| fetal lung-h | | 15 | | | | | | 0 | 666 | 0 | 3,837 | 1,462 | 198,037 | 4,592 | R1 384-1 | sym | A15 | 292 |
| skeletal muscle-h | | 16 | | | | | | 1,336 | 0 | 0 | 2,995 | 0 | 43,019 | 42 | R1 384-1 | sym | A16 | 304 |
| heart-h | | 17 | | | | | | 12,477 | 125 | 0 | 2,160 | 205 | 107,943 | 899 | R1 384-1 | sym | A17 | 293 |
| small intestine-h | | 18 | | | | | | 0 | 226 | 0 | 1,736 | 1,221 | 117,752 | 882 | R1 384-1 | sym | A18 | 305 |
| kidney-h | | 19 | | | | | | 0 | 56 | 0 | 2,418 | 18,839 | 135,359 | 1,267 | R1 384-1 | sym | A19 | 294 |
| spinal cord-h | | 20 | | | | | | 0 | 144 | 0 | 1,031 | 451 | 119,848 | 789 | R1 384-1 | sym | A20 | 306 |
| liver-h | | 21 | | | | | | 379 | 1,481 | 0 | 433 | 0 | 153,048 | 852 | R1 384-1 | sym | A21 | 295 |
| Spleen-h | | 22 | | | | | | 778 | 191 | 0 | 861 | 0 | 122,519 | 1,248 | R1 384-1 | sym | A22 | 307 |
| lung-h | | 23 | | | | | | 0 | 530 | 0 | 1,794 | 0 | 76,316 | 733 | R1 384-1 | sym | A23 | 296 |
| stomach-h | | 24 | | | | | | 372 | 0 | 0 | 171 | 0 | 39,162 | 278 | R1 384-1 | sym | A24 | 308 |
| testis-h | | 25 | | | | | | 15 | 0 | 0 | 14,003 | 0 | 141,552 | 5,293 | R1 384-1 | sym | B01 | 309 |
| thymus-h | | 27 | | | | | | 1,943 | 343 | 0 | 2,504 | 0 | 180,985 | 6,498 | R1 384-1 | sym | B03 | 310 |
| HPAEC | | 28 | | | | 28 | | 0 | 0 | 0 | 2,192 | 0 | 171,007 | 2,973 | R1 384-1 | sym | B04 | 319 |
| thyroid gland-h | | 29 | | | | | | 0 | 561 | 0 | 578 | 0 | 155,558 | 2,783 | R1 384-1 | sym | B05 | 311 |
| RPTEC | | 30 | | | | 30 | | 166 | 170 | 0 | 2,416 | 0 | 40,468 | 0 | R1 384-1 | sym | B06 | 320 |
| trachea-h | | 31 | | | | | | 0 | 5 | 0 | 2,843 | 618 | 126,079 | 3,124 | R1 384-1 | sym | B07 | 312 |
| HMEC | | 32 | | | | | | 0 | 0 | 0 | 0 | 0 | 14,267 | 0 | R1 384-1 | sym | B08 | 321 |
| uterus-h | | 33 | | | | | | 0 | 492 | 0 | 2,700 | 171 | 120,188 | 3,561 | R1 384-1 | sym | B09 | 313 |
| HCAEC | | 34 | | | | | | 495 | 0 | 0 | 0 | 300 | 11,188 | 0 | R1 384-1 | sym | B10 | 322 |
| Pancreas-h | | 35 | | | | | | 0 | 87 | 0 | 482 | 0 | 29,237 | 329 | R1 384-1 | sym | B11 | 314 |
| lymph node-h | | 36 | | | | | | 326 | 298 | 0 | 0 | 0 | 16,454 | 0 | R1 384-1 | sym | B12 | 323 |
| skeletal muscle-h | | 37 | | | | | | 242 | 0 | 0 | 1,216 | 0 | 164,981 | 412 | R1 384-1 | sym | B13 | 315 |
| fetal liver-h | | 38 | | | | | | 0 | 0 | 0 | 506 | 0 | 135,364 | 2,681 | R1 384-1 | sym | B14 | 324 |
| Heart-h | | 39 | | | | | | 500 | 94 | 0 | 2,052 | 0 | 84,824 | 394 | R1 384-1 | sym | B15 | 316 |
| thymus, h | | 40 | | | | | | 0 | 166 | 0 | 145 | 44 | 98,060 | 2,415 | R1 384-1 | sym | B16 | 325 |
| Duodenum-h | | 41 | | | | | | 717 | 113 | 0 | 2,188 | 421 | 70,179 | 559 | R1 384-1 | sym | B17 | 317 |
| Fetal brain-h | | 42 | | | | | | 490 | 0 | 0 | 0 | 0 | 32,869 | 359 | R1 384-1 | sym | B18 | 326 |
| Salivary gl.-h | | 43 | | | | | | 189 | 0 | 0 | 0 | 0 | 45,754 | 196 | R1 384-1 | sym | B19 | 318 |
| testis-h | | 44 | | | | | | 0 | 221 | 0 | 396 | 0 | 83,131 | 273 | R1 384-1 | sym | B20 | 327 |
| fetal kidney-h | | | | | 49 | | | 1,124 | 0 | 0 | 0 | 0 | 67,258 | 423 | R1 384-1 | asym | C01 | 92 |
| fetal liver-h | | | | | 51 | | | 443 | 0 | 0 | 0 | 0 | 18,565 | 208 | R1 384-1 | asym | C03 | 93 |
| fetal lung-h | | | | | 53 | | | 0 | 109 | 0 | 1,903 | 89 | 32,265 | 251 | R1 384-1 | asym | C05 | 94 |
| heart-h | | | | | 55 | | | 0 | 45 | 0 | 268 | 0 | 23,292 | 280 | R1 384-1 | asym | C07 | 95 |
| kidney-h | | | | | 57 | | | 0 | 0 | 0 | 45 | 0 | 84,812 | 1,129 | R1 384-1 | asym | C09 | 96 |
| lung-h | | | | | 59 | | | 0 | 183 | 0 | 0 | 0 | 80,505 | 199 | R1 384-1 | asym | C11 | 98 |
| lymph node-h | | | | | 61 | | | 45 | 0 | 0 | 0 | 40 | 143,506 | 3,584 | R1 384-1 | asym | C13 | 99 |
| WI-38 72h | | | | | 179 | | | 0 | 149 | 0 | 0 | 81 | 17,291 | 327 | R1 384-1 | asym | H11 | 85 |
| thymus-h | | | | | 193 | | | 0 | 0 | 0 | 0 | 659 | 60,103 | 252 | R1 384-1 | asym | I01 | 112 |
| HEPM 3d TGFB1 | | | | | 195 | | | 69 | 134 | 0 | 0 | 0 | 26,232 | 166 | R1 384-1 | asym | I03 | 70 |
| detergent + DNase | | | | | | | | | | | | | | | | | | |
| Salivary gl.-h | | | | | 197 | | | 0 | 0 | 0 | 278 | 0 | 11,196 | 143 | R1 384-1 | asym | I05 | 120 |
| testis-h | | | | | 199 | | | 0 | 86 | 0 | 0 | 17 | 32,630 | 0 | R1 384-1 | asym | I07 | 129 |
| Pancreas-h | | | | | 201 | | | 199 | 0 | 0 | 98 | 0 | 50,700 | 0 | R1 384-1 | asym | I09 | 116 |
| Skeletal muscle-h | | | | | 203 | | | 0 | 0 | 0 | 0 | 32 | 64,559 | 197 | R1 384-1 | asym | I11 | 117 |
| Duodenum-h | | | | | 205 | | | 0 | 0 | 0 | 0 | 0 | 33,654 | 0 | R1 384-1 | asym | I13 | 119 |
| HMEC | | | | | 209 | | | 777 | 102 | 0 | 0 | 206 | 101,094 | 3,485 | R1 384-1 | asym | I17 | 123 |
| fetal brain-h | | | | | 210 | | | 0 | 69 | 0 | 1,050 | 0 | 26,545 | 248 | R1 384-1 | asym | I18 | 191 |
| HCAEC | | | | | 211 | 211 | | 0 | 210 | 0 | 289 | 313 | 106,354 | 2,700 | R1 384-1 | asym | I19 | 124 |
| placenta-h | | | | | 212 | | | 0 | 296 | 0 | 0 | 0 | 16,093 | 223 | R1 384-1 | asym | I20 | 103 |
| fetal liver-h | | | | | 213 | | | 1,358 | 40 | 0 | 0 | 345 | 47,791 | 176 | R1 384-1 | asym | I21 | 126 |
| stomach-h | | | | | 214 | | | 0 | 0 | 0 | 151 | 0 | 26,452 | 196 | R1 384-1 | asym | I22 | 110 |
| Heart-h | | | | | 215 | | | 0 | 0 | 0 | 92 | 0 | 51,494 | 0 | R1 384-1 | asym | I23 | 118 |
| bladder-h | | | | | 222 | | | 0 | 140 | 0 | 0 | 0 | 39,930 | 0 | R1 384-1 | asym | J06 | 130 |
| HT372-normal | | | | | 234 | | | 0 | 50 | 0 | 0 | 0 | 19,299 | 10 | R1 384-1 | asym | J18 | 207 |
| HT382-normal | | | | | 266 | | | 615 | 78 | 0 | 0 | 0 | 86,365 | 202 | R1 384-1 | asym | L02 | 211 |
| HT392-normal | | | | | 268 | | | 131 | 0 | 0 | 0 | 286 | 30,692 | 361 | R1 384-1 | asym | L04 | 214 |
| HPAEC | | | | | 275 | 275 | | 0 | 0 | 0 | 563 | 0 | 40,511 | 422 | R1 384-1 | asym | L11 | 121 |
| adrenal gland-h | | | | | 277 | | | 2,553 | 446 | 166 | 27,369 | 329 | 1,532,778 | 4,110 | R1 384-1 | asym | L13 | 87 |
| bone marrow-h | | | | | 279 | | | 0 | 19 | 0 | 0 | 0 | 6,744 | 114 | R1 384-1 | asym | L15 | 88 |
| skeletal muscle-h | | | | | 290 | | | 0 | 45 | 0 | 1,545 | 0 | 102,189 | 1,579 | R1 384-1 | asym | M02 | 106 |
| small intestine-h | | | | | 292 | | | 0 | 0 | 0 | 0 | 0 | 40,631 | 318 | R1 384-1 | asym | M04 | 107 |
| spinal cord-h | | | | | 294 | | | 0 | 0 | 0 | 357 | 0 | 48,103 | 502 | R1 384-1 | asym | M06 | 108 |
| Spleen-h | | | | | 296 | | | 0 | 3 | 0 | 657 | 153 | 71,535 | 60 | R1 384-1 | asym | M08 | 109 |
| liver-h | | | | | 297 | | | 375 | 0 | 0 | 0 | 0 | 88,510 | 74 | R1 384-1 | asym | M09 | 97 |
| testis-h | | | | | 298 | | | 422 | 0 | 0 | 640 | 0 | 167,195 | 2,423 | R1 384-1 | asym | M10 | 111 |
| bladder-h | | | | | 302 | | | 38 | 311 | 0 | 2,206 | 0 | 74,917 | 825 | R1 384-1 | sym | M14 | 328 |
| mammary gland-h | | | | | 303 | | | 221 | 0 | 0 | 794 | 117 | 48,744 | 0 | R1 384-1 | asym | M15 | 100 |
| pancreas-h | | | | | 305 | | | 678 | 16 | 0 | 817 | 473 | 127,476 | 342 | R1 384-1 | asym | M17 | 101 |
| pituitary gland-h | | | | | 307 | | | 0 | 0 | 0 | 0 | 0 | 20,657 | 0 | R1 384-1 | asym | M19 | 102 |
| prostate, h | | | | | 309 | | | 0 | 634 | 0 | 297 | 2,243 | 148,345 | 3,258 | R1 384-1 | asym | M21 | 104 |
| salivary gl-h | | | | | 311 | | | 455 | 0 | 0 | 1,109 | 0 | 18,958 | 0 | | | | |
| SA-OS (Mundy) | | | | | | | | | | | | | | | R1 384-1 | asym | M23 | 105 |
| poly A+ | | | | | | | | | | | | | | | | | | |
| thyroid gland-h | | | | | 314 | | | 0 | 0 | 0 | 0 | 0 | 127,246 | 72 | R1 384-1 | asym | N02 | 113 |
| trachea-h | | | | | 316 | | | 0 | 125 | 0 | 16 | 172 | 80,358 | 0 | R1 384-1 | asym | N04 | 114 |
| uterus-h | | | | | 318 | | | 19 | 0 | 0 | 1,641 | 0 | 73,044 | 1,084 | R1 384-1 | asym | N06 | 115 |
| HEPM 3d untreated | | | | | 320 | | | 1,065 | 18 | 0 | 0 | 510 | 61,981 | 0 | R1 384-1 | asym | N08 | 71 |
| HT149-normal | | | | | 321 | | | 528 | 0 | 0 | 0 | 0 | 56,128 | 155 | R1 384-1 | asym | N09 | 163 |
| thymus, h | | | | | 326 | | | 0 | 0 | 0 | 0 | 0 | 51,419 | 0 | R1 384-1 | asym | N14 | 127 |
| HT398-normal | | | | | 327 | | | 551 | 431 | 0 | 742 | 0 | 32,502 | 24 | R1 384-1 | asym | N15 | 219 |
| Fetal brain-h | | | | | 328 | | | 734 | 0 | 0 | 1,133 | 0 | 166,205 | 3,822 | R1 384-1 | asym | N16 | 128 |
| h adult SMC | | | | | 330 | | | 0 | 0 | 0 | 0 | 0 | 23,012 | 164 | R1 384-1 | asym | N18 | 75 |
| Oct. 21, 1992 #17 | | | | | | | | | | | | | | | | | | |
| lymph node-h | | | | | 332 | | | 95 | 0 | 0 | 0 | 67 | 22,358 | 0 | R1 384-1 | asym | N20 | 125 |
| RPTEC | | | | | 334 | 334 | | 1,186 | 189 | 0 | 223 | 0 | 96,951 | 2,705 | R1 384-1 | asym | N22 | 122 |
| brain-h | | | | | 342 | | | 0 | 204 | 0 | 0 | 716 | 51,925 | 0 | R1 384-1 | asym | O06 | 89 |
| cerebellum-h | | | | | 344 | | | 0 | 0 | 0 | 40 | 0 | 31,053 | 0 | R1 384-1 | asym | O08 | 90 |
| HT157-normal | | | | | 361 | | | 0 | 285 | 0 | 0 | 0 | 81,782 | 1 | R1 384-1 | asym | P01 | 168 |
| HT213-normal | | | | | 363 | | | 1,947 | 45 | 0 | 15 | 0 | 20,122 | 0 | R1 384-1 | asym | P03 | 181 |
| HT218-normal | | | | | 365 | | | 0 | 0 | 0 | 0 | 0 | 13,192 | 0 | R1 384-1 | asym | P05 | 183 |
| HeLa-1 | | | | | | | HPV E6 | 188 | 27 | 0 | 17,506 | 191 | 555,591 | 1,745 | R2 384-1 | sym | C10 | 394 |
| HeLa-2 | | | | | | | HPV E6 | 362 | 758 | 2 | 52,193 | 0 | 679,609 | 1,613 | R2 384-1 | sym | F18 | 395 |
| HeLa-3 | | | | | | | HPV E6 | 1,051 | 0 | 67 | 19,138 | 0 | 443,018 | 1,179 | R2 384-1 | sym | C12 | 396 |
| HeLa-4 | | | | | | | HPV E6 | 699 | 387 | 0 | 11,837 | 0 | 636,546 | 1,133 | R2 384-1 | sym | C14 | 397 |
| HeLa-5 | | | | | | | HPV E6 | 1,571 | 0 | 600 | 40,632 | 0 | 740,204 | 1,934 | R2 384-1 | sym | C16 | 398 |
| HeLa-6 | | | | | | | HPV E6 | 0 | 0 | 215 | 40,279 | 174 | 849,472 | 3,502 | R2 384-1 | sym | C18 | 399 |
| HeLa-7 | | | | | | | HPV E6 | 233 | 0 | 183 | 39,006 | 81 | 914,876 | 2,315 | R2 384-1 | sym | C20 | 400 |
| HeLa-8 | | | | | | | HPV E6 | 0 | 0 | 0 | 30,268 | 0 | 967,084 | 1,982 | R2 384-1 | sym | C22 | 401 |
| ADR-RES-1 | | | | | | | mutant | 540 | 168 | 0 | 48,622 | 0 | 921,921 | 922 | R2 384-1 | sym | C17 | 341 |
| ADR-RES-2 | | | | | | | mutant | 76 | 0 | 0 | 18,959 | 0 | 1,010,246 | 591 | R2 384-1 | sym | C19 | 342 |
| ADR-RES-3 | | | | | | | mutant | 334 | 344 | 0 | 13,658 | 0 | 359,976 | 383 | R2 384-1 | sym | C21 | 343 |
| ADR-RES-4 | | | | | | | mutant | 0 | 0 | 0 | 8,302 | 0 | 442,971 | 11 | R2 384-1 | sym | C23 | 344 |
| ADR-RES-5 | | | | | | | mutant | 306 | 0 | 164 | 20,910 | 0 | 544,895 | 130 | R2 384-1 | sym | C02 | 345 |
| ADR-RES-6 | | | | | | | mutant | 140 | 0 | 0 | 26,446 | 0 | 601,648 | 468 | R2 384-1 | sym | C04 | 346 |
| ADR-RES-7 | | | | | | | mutant | 0 | 661 | 0 | 1,745 | 429 | 85,849 | 3,468 | R2 384-1 | sym | C06 | 347 |
| ADR-RES-8 | | | | | | | mutant | 0 | 283 | 0 | 3,363 | 0 | 5,866 | 0 | R2 384-1 | sym | C08 | 348 |
| C33A-1 | | | | | | | mutant | 0 | 0 | 0 | 3,630 | 0 | 458,295 | 578 | R2 384-1 | sym | D08 | 349 |
| C33A-2 | | | | | | | mutant | 0 | 0 | 0 | 13,815 | 0 | 389,598 | 609 | R2 384-1 | sym | D10 | 350 |
| C33A-3 | | | | | | | mutant | 4,866 | 0 | 104 | 9,604 | 0 | 379,820 | 824 | R2 384-1 | sym | D12 | 351 |
| C33A-4 | | | | | | | mutant | 89 | 0 | 126 | 8,326 | 42 | 498,796 | 1,112 | R2 384-1 | sym | D14 | 352 |
| C33A-5 | | | | | | | mutant | 0 | 0 | 0 | 34,820 | 0 | 644,383 | 890 | R2 384-1 | sym | D16 | 353 |
| C33A-6 | | | | | | | mutant | 0 | 0 | 0 | 690 | 0 | 678,800 | 0 | R2 384-1 | sym | D18 | 354 |
| C33A-7 | | | | | | | mutant | 0 | 0 | 149 | 18,136 | 0 | 785,878 | 908 | R2 384-1 | sym | D20 | 355 |
| C33A-8 | | | | | | | mutant | 21 | 0 | 173 | 42,512 | 0 | 1,172,556 | 2,189 | R2 384-1 | sym | D22 | 356 |
| EKVX-1 | | | | | | | mutant | 675 | 0 | 0 | 27,186 | 0 | 269,251 | 221 | R2 384-1 | sym | F02 | 370 |
| EKVX-2 | | | | | | | mutant | 0 | 0 | 92 | 69,264 | 272 | 498,108 | 0 | R2 384-1 | sym | F04 | 371 |
| EKVX-3 | | | | | | | mutant | 0 | 4 | 0 | 93,678 | 0 | 429,070 | 340 | R2 384-1 | sym | F06 | 372 |
| EKVX-4 | | | | | | | mutant | 577 | 0 | 71 | 140,696 | 344 | 429,429 | 819 | R2 384-1 | sym | F08 | 373 |
| EKVX-5 | | | | | | | mutant | 29 | 0 | 0 | 34,966 | 0 | 813,745 | 1,003 | R2 384-1 | sym | A17 | 374 |
| EKVX-6 | | | | | | | mutant | 6 | 0 | 98 | 7,689 | 79 | 459,086 | 799 | R2 384-1 | sym | A19 | 375 |
| EKVX-7 | | | | | | | mutant | 705 | 0 | 564 | 41,327 | 393 | 1,353,897 | 2,803 | R2 384-1 | sym | A21 | 376 |
| EKVX-8 | | | | | | | mutant | 192 | 242 | 0 | 31,342 | 0 | 397,494 | 1,472 | R2 384-1 | sym | F10 | 377 |
| H1299-1 | | | | | | | mutant | 1,259 | 0 | 62 | 26,353 | 0 | 501,247 | 1,055 | R2 384-1 | sym | D15 | 378 |
| H1299-2 | | | | | | | mutant | 0 | 327 | 37 | 13,882 | 0 | 712,338 | 1,004 | R2 384-1 | sym | D17 | 379 |
| H1299-3 | | | | | | | mutant | 0 | 121 | 16 | 36,682 | 0 | 778,091 | 2,440 | R2 384-1 | sym | D19 | 380 |
| H1299-4 | | | | | | | mutant | 172 | 0 | 22 | 57,330 | 669 | 957,276 | 2,317 | R2 384-1 | sym | D21 | 381 |
| H1299-5 | | | | | | | mutant | 0 | 0 | 92 | 18,628 | 0 | 865,896 | 1,039 | R2 384-1 | sym | D23 | 382 |
| H1299-6 | | | | | | | mutant | 0 | 79 | 52 | 17,264 | 172 | 338,356 | 412 | R2 384-1 | sym | D02 | 383 |
| H1299-7 | | | | | | | mutant | 147 | 0 | 0 | 12,801 | 0 | 523,628 | 0 | R2 384-1 | sym | D04 | 384 |
| H1299-8 | | | | | | | mutant | 2,032 | 0 | 0 | 21,626 | 553 | 519,154 | 449 | R2 384-1 | sym | D06 | 385 |
| HT29-1 | | | | | | | mutant | 817 | 0 | 85 | 60,106 | 0 | 859,216 | 1,413 | R2 384-1 | sym | A14 | 409 |
| HT29-2 | | | | | | | mutant | 1,145 | 298 | 16 | 62,721 | 0 | 440,697 | 1,568 | R2 384-1 | sym | A16 | 410 |
| HT29-3 | | | | | | | mutant | 833 | 228 | 132 | 85,651 | 228 | 700,898 | 958 | R2 384-1 | sym | A18 | 411 |
| HT29-4 | | | | | | | mutant | 120 | 0 | 258 | 102,716 | 604 | 1,046,464 | 1,512 | R2 384-1 | sym | A20 | 412 |
| HT29-5 | | | | | | | mutant | 810 | 0 | 0 | 137,429 | 0 | 1,522,264 | 3,276 | R2 384-1 | sym | A22 | 413 |
| HT29-6 | | | | | | | mutant | 3,401 | 128 | 154 | 131,385 | 568 | 1,157,087 | 2,892 | R2 384-1 | sym | A24 | 414 |
| HT29-7 | | | | | | | mutant | 0 | 0 | 25 | 21,556 | 455 | 359,692 | 523 | R2 384-1 | sym | B01 | 415 |
| HT29-8 | | | | | | | mutant | 0 | 0 | 227 | 34,495 | 11 | 539,304 | 1,099 | R2 384-1 | sym | B03 | 416 |
| OVCAR-5-1 | | | | | | | mutant | 166 | 0 | 0 | 46,515 | 403 | 407,563 | 1,031 | R2 384-1 | sym | B12 | 454 |
| OVCAR-5-2 | | | | | | | mutant | 0 | 0 | 81 | 22,899 | 15 | 550,448 | 653 | R2 384-1 | sym | B14 | 455 |
| OVCAR-5-3 | | | | | | | mutant | 66 | 344 | 186 | 67,219 | 0 | 386,258 | 33 | R2 384-1 | sym | B16 | 456 |
| OVCAR-5-4 | | | | | | | mutant | 0 | 81 | 0 | 28,479 | 1 | 460,438 | 2,500 | R2 384-1 | sym | B18 | 457 |
| OVCAR-5-5 | | | | | | | mutant | 16 | 314 | 0 | 52,491 | 140 | 607,764 | 883 | R2 384-1 | sym | H03 | 458 |
| OVCAR-5-6 | | | | | | | mutant | 0 | 144 | 41 | 5,128 | 0 | 652,947 | 91 | R2 384-1 | sym | B22 | 459 |
| OVCAR-5-7 | | | | | | | mutant | 1,388 | 743 | 379 | 73,397 | 0 | 2,126,515 | 1,252 | R2 384-1 | sym | B24 | 460 |
| OVCAR-5-8 | | | | | | | mutant | 687 | 0 | 0 | 72,830 | 1,009 | 411,234 | 1,283 | R2 384-1 | sym | F16 | 461 |
| SF-268-1 | | | | | | | mutant | 454 | 0 | 0 | 46,444 | 169 | 1,247,938 | 3,199 | R2 384-1 | sym | E24 | 462 |
| SF-268-2 | | | | | | | mutant | 175 | 0 | 1 | 31,761 | 0 | 294,676 | 519 | R2 384-1 | sym | F01 | 463 |
| SF-268-3 | | | | | | | mutant | 35 | 0 | 211 | 26,186 | 0 | 442,870 | 212 | R2 384-1 | sym | F03 | 464 |
| SF-268-4 | | | | | | | mutant | 178 | 184 | 204 | 51,244 | 0 | 399,799 | 568 | R2 384-1 | sym | F05 | 465 |
| SF-268-5 | | | | | | | mutant | 0 | 816 | 0 | 56,103 | 0 | 471,620 | 0 | R2 384-1 | sym | F07 | 466 |
| SF-268-6 | | | | | | | mutant | 0 | 36 | 0 | 25,570 | 0 | 394,514 | 619 | R2 384-1 | sym | F09 | 467 |
| SF-268-7 | | | | | | | mutant | 850 | 143 | 0 | 40,201 | 429 | 347,362 | 1,837 | R2 384-1 | sym | F11 | 468 |
| SF-268-8 | | | | | | | mutant | 2,391 | 138 | 160 | 67,712 | 12 | 463,334 | 1,254 | R2 384-1 | sym | F13 | 469 |
| SW480-1 | | | | | | | mutant | 0 | 0 | 0 | 26 | 0 | 13,937 | 30 | R2 384-1 | sym | C24 | 478 |
| SW480-2 | | | | | | | mutant | 0 | 255 | 0 | 1,000 | 79 | 33,878 | 1,032 | R2 384-1 | sym | D01 | 479 |
| SW480-3 | | | | | | | mutant | 470 | 0 | 0 | 76,090 | 0 | 1,460,381 | 3,005 | R2 384-1 | sym | D03 | 480 |
| SW480-4 | | | | | | | mutant | 79 | 0 | 0 | 45,130 | 226 | 423,651 | 537 | R2 384-1 | sym | D05 | 481 |
| SW480-5 | | | | | | | mutant | 260 | 85 | 206 | 33,127 | 0 | 392,824 | 235 | R2 384-1 | sym | D07 | 482 |
| SW480-6 | | | | | | | mutant | 733 | 0 | 0 | 11,557 | 0 | 536,855 | 116 | R2 384-1 | sym | D09 | 483 |
| SW480-7 | | | | | | | mutant | 1,608 | 0 | 0 | 38,957 | 0 | 729,185 | 983 | R2 384-1 | sym | D11 | 484 |
| SW480-8 | | | | | | | mutant | 183 | 0 | 0 | 27,056 | 0 | 555,267 | 1,473 | R2 384-1 | sym | D13 | 485 |
| U2OS-1 | | | | | | | mutant | 1,263 | 125 | 0 | 78,276 | 1,905 | 1,630,846 | 4,317 | R2 384-1 | sym | D24 | 486 |
| U2OS-2 | | | | | | | mutant | 641 | 327 | 202 | 29,915 | 0 | 432,337 | 972 | R2 384-1 | sym | E01 | 487 |
| U2OS-3 | | | | | | | mutant | 573 | 0 | 1 | 40,362 | 127 | 406,461 | 412 | R2 384-1 | sym | E03 | 488 |
| U2OS-4 | | | | | | | mutant | 1,545 | 0 | 162 | 10,515 | 106 | 399,039 | 738 | R2 384-1 | sym | E05 | 489 |
| U2OS-5 | | | | | | | mutant | 679 | 0 | 94 | 37,160 | 0 | 491,594 | 827 | R2 384-1 | sym | E07 | 490 |
| U2OS-6 | | | | | | | mutant | 0 | 294 | 108 | 5,845 | 3,862 | 371,040 | 1,175 | R2 384-1 | sym | E09 | 491 |
| U2OS-7 | | | | | | | mutant | 0 | 0 | 0 | 22,921 | 17 | 500,363 | 1,551 | R2 384-1 | sym | E11 | 492 |
| U2OS-8 | | | | | | | mutant | 0 | 0 | 0 | 16,035 | 0 | 472,901 | 1,979 | R2 384-1 | sym | E13 | 493 |
| A549-1 | | | | | | | wt | 0 | 0 | 112 | 54,563 | 0 | 546,984 | 523 | R2 384-1 | sym | A01 | 333 |
| A549-2 | | | | | | | wt | 1,553 | 0 | 0 | 25,728 | 192 | 490,791 | 367 | R2 384-1 | sym | A03 | 334 |
| A549-3 | | | | | | | wt | 0 | 0 | 0 | 8,583 | 0 | 526,257 | 173 | R2 384-1 | sym | A05 | 335 |
| A549-4 | | | | | | | wt | 56 | 118 | 92 | 27,986 | 170 | 637,978 | 439 | R2 384-1 | sym | A07 | 336 |
| A549-5 | | | | | | | wt | 0 | 119 | 0 | 56,834 | 0 | 470,171 | 657 | R2 384-1 | sym | A09 | 337 |
| A549-6 | | | | | | | wt | 0 | 0 | 203 | 2,773 | 0 | 560,302 | 671 | R2 384-1 | sym | A11 | 338 |
| A549-7 | | | | | | | wt | 240 | 0 | 188 | 32,976 | 0 | 658,479 | 1,455 | R2 384-1 | sym | A13 | 339 |
| A549-8 | | | | | | | wt | 57 | 157 | 1 | 74,102 | 243 | 409,754 | 1,458 | R2 384-1 | sym | A15 | 340 |
| HCT-116-1 | | | | | | | wt | 0 | 0 | 0 | 100,001 | 85 | 2,916,547 | 3,159 | R2 384-1 | sym | A23 | 386 |
| HCT-116-2 | | | | | | | wt | 0 | 18 | 4 | 145,047 | 0 | 386,158 | 1,870 | R2 384-1 | sym | F12 | 387 |
| HCT-116-3 | | | | | | | wt | 665 | 0 | 0 | 17,785 | 159 | 427,289 | 313 | R2 384-1 | sym | A02 | 388 |
| HCT-116-4 | | | | | | | wt | 275 | 0 | 0 | 38,703 | 0 | 386,481 | 198 | R2 384-1 | sym | A04 | 389 |
| HCT-116-5 | | | | | | | wt | 556 | 179 | 38 | 37,110 | 85 | 505,899 | 955 | R2 384-1 | sym | A06 | 390 |
| HCT-116-6 | | | | | | | wt | 754 | 0 | 105 | 61,500 | 0 | 498,369 | 488 | R2 384-1 | sym | A08 | 391 |
| HCT-116-7 | | | | | | | wt | 190 | 187 | 47 | 42,722 | 0 | 416,088 | 1,069 | R2 384-1 | sym | A10 | 392 |
| HCT-116-8 | | | | | | | wt | 783 | 0 | 49 | 48,607 | 133 | 555,183 | 1,362 | R2 384-1 | sym | A12 | 393 |
| Hs68-1 | | | | | | | wt | 0 | 0 | 50 | 21,139 | 0 | 412,159 | 1,066 | R2 384-1 | sym | E15 | 402 |
| Hs68-2 | | | | | | | wt | 0 | 84 | 369 | 52,149 | 175 | 562,011 | 1,788 | R2 384-1 | sym | E17 | 403 |
| Hs68-3 | | | | | | | wt | 21 | 0 | 0 | 52,724 | 119 | 832,196 | 1,856 | R2 384-1 | sym | E19 | 404 |
| Hs68-4 | | | | | | | wt | 1,647 | 0 | 0 | 104,514 | 40 | 1,087,919 | 3,110 | R2 384-1 | sym | E21 | 405 |
| Hs68-6 | | | | | | | wt | 0 | 0 | 47 | 6,341 | 425 | 271,530 | 521 | R2 384-1 | sym | E02 | 406 |
| Hs68-7 | | | | | | | wt | 367 | 0 | 0 | 25,147 | 0 | 491,608 | 246 | R2 384-1 | sym | E04 | 407 |
| Hs68-8 | | | | | | | wt | 0 | 16 | 0 | 30,704 | 0 | 498,513 | 192 | R2 384-1 | sym | E06 | 408 |
| MCF-7-1 | | | | | | | wt | 0 | 0 | 0 | 7,662 | 0 | 604,049 | 344 | R2 384-1 | sym | C01 | 417 |
| MCF-7-2 | | | | | | | wt | 1,092 | 0 | 141 | 26,075 | 0 | 662,630 | 341 | R2 384-1 | sym | C03 | 418 |
| MCF-7-3 | | | | | | | wt | 1,349 | 0 | 341 | 17,550 | 0 | 632,258 | 621 | R2 384-1 | sym | C05 | 419 |
| MCF-7-4 | | | | | | | wt | 275 | 6 | 0 | 20,701 | 0 | 661,141 | 1,174 | R2 384-1 | sym | C07 | 420 |
| MCF-7-5 | | | | | | | wt | 0 | 0 | 0 | 8,565 | 237 | 484,097 | 737 | R2 384-1 | sym | C09 | 421 |
| MCF-7-6 | | | | | | | wt | 0 | 0 | 0 | 20,587 | 386 | 795,802 | 1,446 | R2 384-1 | sym | C11 | 422 |
| MCF-7-7 | | | | | | | wt | 0 | 0 | 0 | 0 | 357 | 33,599 | 0 | R2 384-1 | sym | C13 | 423 |
| MCF-7-8 | | | | | | | wt | 560 | 0 | 0 | 2,415 | 460 | 76,817 | 535 | R2 384-1 | sym | C15 | 424 |
| OVCAR-4-1 | | | | | | | wt | 1,159 | 0 | 126 | 65,996 | 43 | 777,026 | 2,150 | R2 384-1 | sym | B21 | 446 |
| OVCAR-4-2 | | | | | | | wt | 0 | 479 | 0 | 48,716 | 0 | 3,789,726 | 5,436 | R2 384-1 | sym | B23 | 447 |
| OVCAR-4-3 | | | | | | | wt | 0 | 314 | 77 | 29,888 | 0 | 417,174 | 388 | R2 384-1 | sym | B02 | 448 |
| OVCAR-4-4 | | | | | | | wt | 0 | 118 | 0 | 10,373 | 0 | 477,283 | 117 | R2 384-1 | sym | B04 | 449 |
| OVCAR-4-5 | | | | | | | wt | 396 | 123 | 89 | 38,345 | 0 | 581,094 | 506 | R2 384-1 | sym | B06 | 450 |
| OVCAR-4-6 | | | | | | | wt | 462 | 56 | 0 | 41,745 | 0 | 600,108 | 457 | R2 384-1 | sym | B08 | 451 |
| OVCAR-4-7 | | | | | | | wt | 59 | 0 | 208 | 7,508 | 356 | 427,310 | 554 | R2 384-1 | sym | B10 | 452 |
| OVCAR-4-8 | | | | | | | wt | 278 | 0 | 121 | 88,152 | 0 | 471,191 | 924 | R2 384-1 | sym | H01 | 453 |
| SF539-1 | | | | | | | wt | 0 | 283 | 92 | 11,389 | 0 | 509,238 | 91 | R2 384-1 | sym | B05 | 470 |
| SF539-2 | | | | | | | wt | 277 | 145 | 0 | 9,827 | 11 | 651,624 | 427 | R2 384-1 | sym | B07 | 471 |
| SF539-3 | | | | | | | wt | 1,599 | 0 | 0 | 21,929 | 0 | 547,965 | 799 | R2 384-1 | sym | B09 | 472 |
| SF539-4 | | | | | | | wt | 0 | 179 | 0 | 19,159 | 0 | 449,192 | 668 | R2 384-1 | sym | B11 | 473 |
| SF539-5 | | | | | | | wt | 85 | 0 | 323 | 23,329 | 0 | 516,343 | 319 | R2 384-1 | sym | B13 | 474 |
| SF539-6 | | | | | | | wt | 0 | 108 | 0 | 7,791 | 0 | 662,214 | 668 | R2 384-1 | sym | B15 | 475 |
| SF539-7 | | | | | | | wt | 0 | 0 | 290 | 17,423 | 0 | 574,107 | 1,461 | R2 384-1 | sym | B17 | 476 |
| SF539-8 | | | | | | | wt | 0 | 58 | 0 | 38,279 | 0 | 550,442 | 1,574 | R2 384-1 | sym | B19 | 477 |
| WI 38-1 | | | | | | | wt | 391 | 90 | 25 | 54,921 | 0 | 498,076 | 672 | R2 384-1 | sym | E08 | 494 |
| WI 38-2 | | | | | | | wt | 725 | 671 | 339 | 93,773 | 270 | 339,104 | 2,179 | R2 384-1 | sym | E10 | 495 |
| WI 38-3 | | | | | | | wt | 1,101 | 524 | 65 | 40,084 | 0 | 414,523 | 945 | R2 384-1 | sym | E12 | 496 |
| WI 38-4 | | | | | | | wt | 6 | 752 | 0 | 37,054 | 0 | 620,563 | 661 | R2 384-1 | sym | E14 | 497 |
| WI 38-5 | | | | | | | wt | 0 | 289 | 0 | 7,149 | 0 | 552,397 | 1,748 | R2 384-1 | sym | E16 | 498 |
| WI 38-6 | | | | | | | wt | 161 | 0 | 0 | 10,543 | 0 | 559,003 | 724 | R2 384-1 | sym | E18 | 499 |
| WI 38-7 | | | | | | | wt | 385 | 63 | 92 | 31,968 | 0 | 832,476 | 1,298 | R2 384-1 | sym | E20 | 500 |
| WI 38-8 | | | | | | | wt | 80 | 157 | 0 | 5,997 | 0 | 1,071,262 | 1,559 | R2 384-1 | sym | E22 | 501 |
| HELA-2h-031899 | | | | 79 | | | | 533 | 0 | 0 | 680 | 127 | 61,571 | 1,378 | R1 384-1 | asym | D07 | 148 |
| HELA-4h-031899 | | | | 81 | | | | 0 | 0 | 0 | 1,627 | 0 | 96,196 | 929 | R1 384-1 | asym | D09 | 149 |
| HELA-9h-031899 | | | | 83 | | | | 0 | 232 | 0 | 174 | 0 | 91,313 | 810 | R1 384-1 | asym | D11 | 152 |
| HELA-0h-031899 | | | | 86 | | | | 0 | 0 | 0 | 293 | 0 | 41,904 | 164 | R1 384-1 | asym | D14 | 147 |
| HELA-6h-031899 | | | | 88 | | | | 305 | 0 | 0 | 462 | 0 | 56,525 | 291 | R1 384-1 | asym | D16 | 150 |
| HELA-8h-031899 | | | | 90 | | | | 0 | 0 | 0 | 0 | 0 | 80,592 | 531 | R1 384-1 | asym | D18 | 151 |
| HELA-10h-031899 | | | | 92 | | | | 0 | 0 | 0 | 2,217 | 0 | 50,148 | 0 | R1 384-1 | asym | D20 | 153 |
| HELA-11h-031899 | | | | 94 | | | | 0 | 0 | 0 | 0 | 0 | 45,767 | 0 | R1 384-1 | asym | D22 | 154 |
| HELA-12h-031899 | | | | 96 | | | | 496 | 0 | 0 | 231 | 0 | 43,735 | 297 | R1 384-1 | asym | D24 | 155 |
| NCI-H322M | | | | 146 | | | | 0 | 138 | 0 | 1,805 | 0 | 69,902 | 1,926 | R1 384-1 | asym | G02 | 5 |
| NCI-H460 | | | | 148 | | | | 528 | 0 | 0 | 0 | 432 | 41,427 | 0 | R1 384-1 | asym | G04 | 6 |
| NCI-H522 | | | | 150 | | | | 0 | 101 | 0 | 0 | 960 | 64,259 | 1,627 | R1 384-1 | asym | G06 | 7 |
| SNB-19 | | | | 152 | | | | 515 | 0 | 0 | 0 | 217 | 15,805 | 214 | R1 384-1 | asym | G08 | 16 |
| SNB-75 | | | | 154 | | | | 10 | 0 | 0 | 1,207 | 0 | 36,729 | 0 | R1 384-1 | asym | G10 | 17 |
| SF-268 | | | | 156 | | | | 405 | 0 | 0 | 0 | 0 | 11,197 | 354 | R1 384-1 | asym | G12 | 19 |
| SF-295 | | | | 158 | | | | 612 | 630 | 0 | 760 | 532 | 43,210 | 1,364 | R1 384-1 | asym | G14 | 20 |
| CCRF-CEM | | | | 160 | | | | 0 | 0 | 0 | 599 | 0 | 2,830 | 40 | R1 384-1 | asym | G16 | 22 |
| DU-145 | | | | 162 | | | | 148 | 0 | 0 | 0 | 0 | 55,584 | 970 | R1 384-1 | asym | G18 | 28 |
| HCT 116 | | | | 164 | | | | 876 | 0 | 0 | 171 | 604 | 48,404 | 640 | R1 384-1 | asym | G20 | 31 |
| Caki-1 | | | | 166 | | | | 1,789 | 63 | 0 | 195 | 357 | 9,678 | 0 | R1 384-1 | asym | G22 | 39 |
| 786-0 | | | | 168 | | | | 847 | 114 | 0 | 558 | 0 | 5,791 | 48 | R1 384-1 | asym | G24 | 42 |
| T-47D | | | | 169 | | | | 404 | 55 | 0 | 1,859 | 0 | 13,446 | 531 | R1 384-1 | asym | H01 | 58 |
| CRL1441 RNA 8/30 | | | | 181 | | | | 1,738 | 0 | 0 | 0 | 590 | 4,867 | 0 | R1 384-1 | asym | H13 | 79 |
| 781T untreated + | | | | 183 | | | | 1,066 | 16 | 0 | 0 | 52 | 4,220 | 168 | R1 384-1 | asym | H15 | 73 |
| DNase | | | | | | | | | | | | | | | | | | |
| KB poly A+ | | | | 194 | | | | 452 | 357 | 0 | 0 | 219 | 43,402 | 0 | R1 384-1 | asym | I02 | 59 |
| HOS poly A+ | | | | 196 | | | | 0 | 0 | 0 | 0 | 904 | 58,885 | 468 | R1 384-1 | asym | I04 | 62 |
| ACHN | | | | 198 | | | | 426 | 236 | 0 | 193 | 3,740 | 67,534 | 748 | R1 384-1 | asym | I06 | 41 |
| UACC-62 | | | | 200 | | | | 0 | 282 | 0 | 459 | 7 | 12,401 | 0 | R1 384-1 | asym | I08 | 49 |
| MCF-7/ADR-RES | | | | 202 | | | | 776 | 0 | 0 | 209 | 0 | 44,167 | 0 | R1 384-1 | asym | I10 | 53 |
| UTOS (Mundy) | | | | 204 | | | | 0 | 143 | 0 | 373 | 359 | 13,394 | 115 | R1 384-1 | asym | I12 | 65 |
| poly a+ | | | | | | | | | | | | | | | | | | |
| WISH (Collagen) | | | | 206 | | | | 1,552 | 0 | 0 | 0 | 0 | 3,311 | 0 | R1 384-1 | asym | I14 | 66 |
| poly A+ | | | | | | | | | | | | | | | | | | |
| 458 medullo mRNA | | | | 208 | | | | 0 | 290 | 0 | 4,761 | 0 | 38,074 | 0 | R1 384-1 | asym | I16 | 69 |
| CCL137 RNA | | | | 218 | | | | 0 | 0 | 0 | 392 | 65 | 35,013 | 425 | R1 384-1 | asym | J02 | 77 |
| Mar. 21, 1988 | | | | | | | | | | | | | | | | | | |
| WI-38 72h 0.5% | | | | 219 | | | | 0 | 0 | 0 | 0 | 0 | 43,199 | 0 | R1 384-1 | asym | J03 | 86 |
| FBS, 24h 10% FBS | | | | | | | | | | | | | | | | | | |
| CRL 1441 + TPA | | | | 220 | | | | 0 | 56 | 0 | 386 | 0 | 13,416 | 59 | R1 384-1 | asym | J04 | 78 |
| (24h) 8/30 | | | | | | | | | | | | | | | | | | |
| HOP-92 | | | | 241 | | | | 0 | 23 | 0 | 0 | 0 | 22,647 | 186 | R1 384-1 | asym | K01 | 1 |
| MOLT-4 | | | | 242 | | | | 0 | 92 | 0 | 309 | 26 | 21,250 | 434 | R1 384-1 | asym | K02 | 24 |
| EKVX | | | | 243 | | | | 0 | 250 | 0 | 960 | 0 | 77,320 | 2,875 | R1 384-1 | asym | K03 | 2 |
| HL-60 | | | | 244 | | | | 0 | 0 | 0 | 908 | 480 | 24,392 | 0 | R1 384-1 | asym | K04 | 25 |
| NCI-H23 | | | | 245 | | | | 1,129 | 0 | 0 | 211 | 0 | 93,263 | 2,462 | R1 384-1 | asym | K05 | 3 |
| RPMI 8226 | | | | 246 | | | | 0 | 0 | 0 | 799 | 0 | 87,784 | 1,931 | R1 384-1 | asym | K06 | 26 |
| A549/ATCC | | | | 247 | | | | 226 | 0 | 0 | 0 | 211 | 5,669 | 0 | R1 384-1 | asym | K07 | 8 |
| SR | | | | 248 | | | | 329 | 0 | 0 | 0 | 0 | 15,586 | 296 | R1 384-1 | asym | K08 | 27 |
| OVCAR-3 | | | | 249 | | | | 112 | 0 | 0 | 0 | 594 | 15,287 | 240 | R1 384-1 | asym | K09 | 10 |
| HCT-15 | | | | 250 | | | | 284 | 387 | 0 | 2,254 | 94 | 30,611 | 211 | R1 384-1 | asym | K10 | 34 |
| OVCAR-4 | | | | 251 | | | | 0 | 278 | 0 | 0 | 0 | 4,121 | 166 | R1 384-1 | asym | K11 | 11 |
| UO-31 | | | | 252 | | | | 0 | 0 | 0 | 0 | 30 | 7,831 | 96 | R1 384-1 | asym | K12 | 36 |
| OVCAR-5 | | | | 253 | | | | 181 | 265 | 0 | 1,579 | 756 | 28,038 | 1,658 | R1 384-1 | asym | K13 | 12 |
| SN12C | | | | 254 | | | | 0 | 151 | 0 | 0 | 0 | 5,978 | 0 | R1 384-1 | asym | K14 | 37 |
| OVCAR-8 | | | | 255 | | | | 0 | 7 | 0 | 952 | 591 | 67,456 | 1,954 | R1 384-1 | asym | K15 | 13 |
| LOX IMVI | | | | 256 | | | | 239 | 624 | 0 | 2,645 | 954 | 25,761 | 1,176 | R1 384-1 | asym | K16 | 44 |
| IGROV1 | | | | 257 | | | | 0 | 0 | 0 | 1,111 | 3 | 56,485 | 1,865 | R1 384-1 | asym | K17 | 14 |
| SK-MEL-2 | | | | 258 | | | | 0 | 0 | 0 | 0 | 0 | 2,107 | 619 | R1 384-1 | asym | K18 | 46 |
| SK-OV-3 | | | | 259 | | | | 350 | 0 | 0 | 0 | 0 | 3,010 | 95 | R1 384-1 | asym | K19 | 15 |
| SK-MEL-5 | | | | 260 | | | | 0 | 0 | 0 | 0 | 0 | 1,573 | 0 | R1 384-1 | asym | K20 | 47 |
| SF-539 | | | | 261 | | | | 726 | 0 | 0 | 869 | 0 | 9,243 | 202 | R1 384-1 | asym | K21 | 21 |
| SK-MEL-28 | | | | 262 | | | | 148 | 0 | 0 | 1,157 | 273 | 23,788 | 634 | R1 384-1 | asym | K22 | 48 |
| K-562 | | | | 263 | | | | 313 | 683 | 0 | 763 | 597 | 4,477 | 300 | R1 384-1 | asym | K23 | 23 |
| UACC-257 | | | | 264 | | | | 245 | 0 | 0 | 26 | 0 | 2,255 | 330 | R1 384-1 | asym | K24 | 50 |
| M14 | | | | 265 | | | | 90 | 242 | 0 | 0 | 214 | 129,614 | 1,060 | R1 384-1 | asym | L01 | 51 |
| MCF7 | | | | 267 | | | | 204 | 0 | 0 | 16,366 | 0 | 432,526 | 111 | R1 384-1 | asym | L03 | 52 |
| MDA-MB-435 | | | | 269 | | | | 90 | 0 | 0 | 1,131 | 0 | 14,733 | 0 | R1 384-1 | asym | L05 | 56 |
| HT279 | | | | 270 | | | | 0 | 323 | 0 | 0 | 0 | 19,273 | 86 | R1 384-1 | asym | L06 | 185 |
| MDA-N | | | | 271 | | | | 12 | 176 | 0 | 89 | 0 | 9,135 | 317 | R1 384-1 | asym | L07 | 57 |
| Y79 poly A+ | | | | 273 | | | | 463 | 107 | 0 | 0 | 20 | 69,877 | 0 | R1 384-1 | asym | L09 | 61 |
| KHOS poly A+ | | | | 289 | | | | 0 | 0 | 0 | 0 | 0 | 75,507 | 0 | R1 384-1 | asym | M01 | 63 |
| HTB36 24h TPA | | | | 300 | | | | 5 | 50 | 0 | 0 | 86 | 34,541 | 0 | R1 384-1 | asym | M12 | 82 |
| RNA 6/23 | | | | | | | | | | | | | | | | | | |
| HELA-EXP-031899 | | | | 313 | | | | 0 | 0 | 0 | 110 | 0 | 55,070 | 45 | R1 384-1 | asym | N01 | 156 |
| HTB36 0h RNA | | | | 322 | | | | 0 | 20 | 0 | 0 | 0 | 49,195 | 54 | R1 384-1 | asym | N10 | 72 |
| HT347 | | | | 323 | | | | 0 | 282 | 0 | 521 | 0 | 46,274 | 127 | R1 384-1 | asym | N11 | 200 |
| 458 medullo RNA | | | | 324 | | | | 0 | 0 | 0 | 0 | 0 | 26,987 | 60 | R1 384-1 | asym | N12 | 74 |
| NCI-H226 | | | | 336 | | | | 214 | 352 | 0 | 0 | 257 | 2,962 | 0 | R1 384-1 | asym | N24 | 4 |
| HOP-62 | | | | 337 | | | | 219 | 0 | 0 | 0 | 0 | 11,386 | 157 | R1 384-1 | asym | O01 | 9 |
| MDA-MB-231 | | | | 338 | | | | 0 | 0 | 0 | 577 | 277 | 51,134 | 901 | R1 384-1 | asym | O02 | 55 |
| U251 | | | | 339 | | | | 0 | 237 | 0 | 4,549 | 515 | 55,589 | 372 | R1 384-1 | asym | O03 | 18 |
| PT cells poly A+ | | | | 340 | | | | 130 | 106 | 0 | 0 | 261 | 9,973 | 0 | R1 384-1 | asym | O04 | 60 |
| PC-3 | | | | 341 | | | | 0 | 284 | 0 | 0 | 1,140 | 65,936 | 1,366 | R1 384-1 | asym | O05 | 29 |
| HCC-2998 | | | | 343 | | | | 803 | 0 | 0 | 375 | 639 | 118,307 | 958 | R1 384-1 | asym | O07 | 30 |
| SW-620 | | | | 345 | | | | 0 | 0 | 79 | 22,607 | 0 | 467,592 | 1,300 | | | | |
| SW480-7 | | | | | | | | | | | | | | | R1 384-1 | asym | O09 | 32 |
| HT192 | | | | 346 | | | | 0 | 0 | 0 | 0 | 0 | 74,830 | 83 | R1 384-1 | asym | O10 | 179 |
| COLO 205 | | | | 347 | | | | 621 | 6 | 0 | 0 | 0 | 10,126 | 0 | R1 384-1 | asym | O11 | 33 |
| HT218 | | | | 348 | | | | 0 | 0 | 0 | 0 | 0 | 20,377 | 0 | R1 384-1 | asym | O12 | 182 |
| KM-12 | | | | 349 | | | | 860 | 259 | 0 | 623 | 0 | 21,590 | 0 | R1 384-1 | asym | O13 | 35 |
| HT151 | | | | 350 | | | | 0 | 411 | 0 | 347 | 0 | 60,081 | 414 | R1 384-1 | asym | O14 | 164 |
| A498 | | | | 351 | | | | 0 | 428 | 0 | 0 | 378 | 9,226 | 0 | R1 384-1 | asym | O15 | 38 |
| HT393 | | | | 352 | | | | 0 | 0 | 0 | 694 | 0 | 57,432 | 0 | R1 384-1 | asym | O16 | 215 |
| RXF 393 | | | | 353 | | | | 0 | 0 | 0 | 0 | 0 | 6,338 | 82 | R1 384-1 | asym | O17 | 40 |
| TK-10 | | | | 355 | | | | 191 | 0 | 0 | 2,314 | 0 | 14,179 | 0 | R1 384-1 | asym | O19 | 43 |
| Malme-3M | | | | 357 | | | | 0 | 0 | 0 | 1,531 | 885 | 10,560 | 174 | R1 384-1 | asym | O21 | 45 |
| Hs 578T | | | | 359 | | | | 212 | 0 | 0 | 0 | 9 | 2,568 | 0 | R1 384-1 | asym | O23 | 54 |
| HT213 | | | 50 | | | | | 886 | 0 | 0 | 0 | 0 | 48,907 | 0 | R1 384-1 | asym | C02 | 180 |
| HT288 | | | 52 | | | | | 0 | 224 | 0 | 693 | 0 | 74,176 | 18 | R1 384-1 | asym | C04 | 187 |
| HT139 | | | 54 | | | | | 0 | 0 | 0 | 631 | 0 | 67,320 | 102 | R1 384-1 | asym | C06 | 158 |
| HT155 | | | 56 | | | | | 0 | 0 | 0 | 30 | 0 | 61,739 | 0 | R1 384-1 | asym | C08 | 166 |
| HT163 | | | 58 | | | | | 48 | 0 | 0 | 0 | 345 | 80,453 | 275 | R1 384-1 | asym | C10 | 171 |
| HT170 | | | 60 | | | | | 0 | 184 | 0 | 773 | 0 | 63,967 | 0 | R1 384-1 | asym | C12 | 173 |
| HT172 | | | 62 | | | | | 0 | 0 | 0 | 0 | 0 | 48,537 | 0 | R1 384-1 | asym | C14 | 174 |
| HT138 | | | 63 | | | | | 10 | 1 | 0 | 741 | 0 | 46,416 | 156 | R1 384-1 | asym | C15 | 157 |
| HT178 | | | 64 | | | | | 0 | 14 | 0 | 301 | 0 | 57,882 | 330 | R1 384-1 | asym | C16 | 175 |
| HT154 | | | 65 | | | | | 0 | 166 | 0 | 0 | 0 | 69,411 | 89 | R1 384-1 | asym | C17 | 165 |
| HT180 | | | 66 | | | | | 47 | 57 | 0 | 0 | 0 | 53,278 | 214 | R1 384-1 | asym | C18 | 176 |
| HT169 | | | 67 | | | | | 0 | 0 | 0 | 0 | 0 | 23,349 | 0 | R1 384-1 | asym | C19 | 172 |
| HT189 | | | 68 | | | | | 182 | 0 | 0 | 0 | 0 | 63,765 | 243 | R1 384-1 | asym | C20 | 177 |
| HT143 | | | 69 | | | | | 0 | 0 | 0 | 86 | 0 | 43,069 | 129 | R1 384-1 | asym | C21 | 160 |
| HT190 | | | 70 | | | | | 0 | 0 | 0 | 10 | 0 | 51,091 | 22 | R1 384-1 | asym | C22 | 178 |
| HT145 | | | 71 | | | | | 372 | 0 | 0 | 0 | 0 | 58,721 | 0 | R1 384-1 | asym | C23 | 161 |
| HT227 | | | 72 | | | | | 101 | 0 | 0 | 173 | 29 | 33,093 | 0 | R1 384-1 | asym | C24 | 184 |
| HT302 | | | 73 | | | | | 56 | 207 | 0 | 0 | 0 | 69,267 | 275 | R1 384-1 | asym | D01 | 188 |
| HT314 | | | 74 | | | | | 0 | 20 | 0 | 1,141 | 0 | 60,939 | 287 | R1 384-1 | asym | D02 | 193 |
| HT317 | | | 76 | | | | | 0 | 0 | 0 | 0 | 0 | 51,326 | 484 | R1 384-1 | asym | D04 | 194 |
| Medulloblastoma | | | 77 | | | | | 0 | 74 | 0 | 774 | 0 | 76,589 | 60 | R1 384-1 | asym | D05 | 83 |
| #425 11/8 | | | | | | | | | | | | | | | | | | |
| HT323 | | | 78 | | | | | 0 | 0 | 0 | 0 | 0 | 111,765 | 339 | R1 384-1 | asym | D06 | 195 |
| HT327 | | | 80 | | | | | 0 | 251 | 0 | 691 | 432 | 62,791 | 338 | R1 384-1 | asym | D08 | 196 |
| HT335 | | | 82 | | | | | 0 | 25 | 0 | 176 | 0 | 69,031 | 0 | R1 384-1 | asym | D10 | 198 |
| HT146 | | | 85 | | | | | 227 | 0 | 0 | 0 | 405 | 7,160 | 0 | R1 384-1 | asym | D13 | 162 |
| HT348 | | | 87 | | | | | 359 | 0 | 0 | 0 | 0 | 69,239 | 1,165 | R1 384-1 | asym | D15 | 201 |
| HT311 | | | 170 | | | | | 0 | 75 | 0 | 0 | 0 | 31,571 | 246 | R1 384-1 | asym | H02 | 191 |
| HT398 | | | 185 | | | | | 3,484 | 281 | 0 | 0 | 259 | 5,903 | 31 | R1 384-1 | asym | H17 | 218 |
| HT140 | | | 187 | | | | | 662 | 0 | 0 | 0 | 0 | 1,081 | 0 | R1 384-1 | asym | H19 | 159 |
| HT281 | | | 189 | | | | | 549 | 0 | 0 | 0 | 231 | 5,431 | 0 | R1 384-1 | asym | H21 | 186 |
| HT372 | | | 191 | | | | | 236 | 0 | 0 | 0 | 0 | 11,267 | 0 | R1 384-1 | asym | H23 | 206 |
| TCGP | | | 207 | | | | | 0 | 0 | 0 | 0 | 265 | 9,599 | 16 | R1 384-1 | asym | I15 | 76 |
| HT160 | | | 216 | | | | | 0 | 0 | 0 | 0 | 0 | 2,822 | 0 | R1 384-1 | asym | I24 | 169 |
| HT307 | | | 217 | | | | | 144 | 149 | 0 | 0 | 0 | 27,812 | 84 | R1 384-1 | asym | J01 | 189 |
| HT369 | | | 224 | | | | | 0 | 103 | 0 | 0 | 0 | 45,642 | 0 | R1 384-1 | asym | J08 | 203 |
| HT370 | | | 226 | | | | | 0 | 130 | 0 | 0 | 0 | 33,703 | 22 | R1 384-1 | asym | J10 | 204 |
| HT371 | | | 228 | | | | | 0 | 58 | 0 | 576 | 0 | 46,710 | 453 | R1 384-1 | asym | J12 | 205 |
| HT377 | | | 230 | | | | | 0 | 132 | 0 | 0 | 0 | 35,666 | 354 | R1 384-1 | asym | J14 | 208 |
| HT382 | | | 236 | | | | | 101 | 328 | 0 | 0 | 0 | 28,197 | 45 | R1 384-1 | asym | J20 | 210 |
| neruoblastoma RNA | | | 281 | | | | | 1,184 | 396 | 0 | 2,805 | 0 | 0 | 0 | R1 384-1 | asym | L17 | 84 |
| HT334 | | | 299 | | | | | 0 | 0 | 0 | 0 | 0 | 111,643 | 377 | R1 384-1 | asym | M11 | 197 |
| HT338 | | | 301 | | | | | 0 | 56 | 0 | 0 | 0 | 28,893 | 0 | R1 384-1 | asym | M13 | 199 |
| HT392 | | | 315 | | | | | 460 | 0 | 0 | 0 | 0 | 19,458 | 155 | R1 384-1 | asym | N03 | 213 |
| HT394 | | | 317 | | | | | 0 | 0 | 0 | 46 | 62 | 37,089 | 235 | R1 384-1 | asym | N05 | 216 |
| HT312 | | | 319 | | | | | 181 | 36 | 0 | 0 | 245 | 28,897 | 0 | R1 384-1 | asym | N07 | 192 |
| HT162 | | | 325 | | | | | 0 | 0 | 0 | 0 | 0 | 17,011 | 0 | R1 384-1 | asym | N13 | 170 |
| HT395 | | | 358 | | | | | 233 | 0 | 0 | 0 | 0 | 7,637 | 39 | R1 384-1 | asym | O22 | 217 |
| HT157 | | | 360 | | | | | 0 | 383 | 0 | 0 | 0 | 13,226 | 0 | R1 384-1 | asym | O24 | 167 |
| 458 medullo RNA | | | | | | | | 138 | 154 | 0 | 163 | 0 | 90,329 | 690 | R1 384-1 | sym | B21 | 282 |
| CRL 1572 | | | | | | | | 0 | 0 | 0 | 248 | 0 | 56,547 | 0 | R1 384-1 | asym | D03 | 80 |
| Mar. 17, 1989 | | | | | | | | | | | | | | | | | | |
| h fibroblasts | | | | | | | | 423 | 390 | 0 | 0 | 102 | 12,530 | 184 | R1 384-1 | asym | L21 | 132 |
| Mar. 31, 1992 #12 | | | | | | | | | | | | | | | | | | |
| h keratinocytes | | | | | | | | 64 | 0 | 0 | 0 | 419 | 15,538 | 0 | R1 384-1 | asym | J16 | 133 |
| Feb. 25, 1992 #10 | | | | | | | | | | | | | | | | | | |
| HT308 | | | | | | | | 899 | 0 | 0 | 0 | 0 | 54,950 | 21 | R1 384-1 | asym | D23 | 190 |
| HT368 | | | | | | | | 0 | 31 | 0 | 0 | 80 | 66,952 | 0 | R1 384-1 | asym | D17 | 202 |
| HT378 | | | | | | | | 0 | 0 | 0 | 40 | 0 | 56,777 | 707 | R1 384-1 | asym | D19 | 209 |
| HT385 | | | | | | | | 0 | 0 | 0 | 846 | 0 | 33,000 | 0 | R1 384-1 | asym | D21 | 212 |

| Tissue_type | CC | DD | MC | SR | ANG | PRO | Mets | Prostate | Neuro | Heme | Serum | Mitosis | Cell Cycle | DNA Dam | Ken Lip | Angio | NCI-Ras | Hypoxia | PYK2 | “Diabetes” |
|---|---|---|---|---|---|---|---|---|---|---|---|---|---|---|---|---|---|---|---|---|
| TCGP | | | | | | | | | | | | | 26 | | | | | | | |
| h keratinocytes | | | | | | | | | | | | | | | | | | | | |
| Feb 25, 1992 #10 | | | | | | | | | | | | | | | | | | | | |
| h adult SMC | | | | | | | | | | | | | | | | | | | | |
| Oct. 21, 1992 #17 | | | | | | | | | | | | | | | | | | | | |
| h fibroblasts | | | | | | | | | | | | | | | | | | | | |
| Mar. 31, 1992 #12 | | | | | | | | | | | | | | | | | | | | |
| HOP-92 | | | | | | | | | | | | | | | | | NL | | | |
| OVCAR-8 | | | | | | | | | | | | | | | | | | | | |
| EKVX | | | | | | | | | | | | | | | | | | | | |
| IGROV1 | | | | | | | | | | | | | | | | | NL | | | |
| NCI-H23 | | | | | | | | | | | | | | | | | | | | |
| SK-OV-3 | | | | | | | | | | | | | | | | | | | | |
| NCI-H226 | | | | | | | | | | | | | | | | | | | | |
| SNB-19 | | | | | | | | | 104 | | | | | | | | NL | | | |
| NCI-H322M | | | | | | | | | | | | | | | | | NL | | | |
| SNB-75 | | | | | | | | | 106 | | | | | | | | NL | | | |
| NCI-H460 | | | | | | | | | | | | | | | | | KRas61 | | | |
| U251 | | | | | | | | | 108 | | | | | | | | NL | | | |
| NCI-H522 | | | | | | | | | | | | | | | | | | | | |
| SF-268 | | | | | | | | | 110 | | | | | | | | | | | |
| A549/ATCC | | | | | | | | | | | | | | | | | | | | |
| SF-295 | | | | | | | | | 112 | | | | | | | | NL | | | |
| HOP-62 | | | | | | | | | | | | | | | | | | | | |
| SF-539 | | | | | | | | | | | | | | | | | NL | | | |
| OVCAR-3 | | | | | | | | | 114 | | | | | | | | | | | |
| CCRF-CEM | | | | | | | | | | | | | | | | | | | | |
| OVCAR-4 | | | | | | | | | | | | | | | | | NL | | | |
| K-562 | | | | | | | | | | 118 | | | | | | | | | | |
| OVCAR-5 | | | | | | | | | | | | | | | | | KRas12 | | | |
| MOLT-4 | | | | | | | | | | 120 | | | | | | | | | | |
| HL-60 | | | | | | | | | | 121 | | | | | | | | | | |
| SN12C | | | | | | | | | | | | | | | | | | | | |
| RPMI 8226 | | | | | | | | | | 123 | | | | | | | KRas12 | | | |
| A498 | | | | | | | | | | | | | | | | | | | | |
| SR | | | | | | | | | | 125 | | | | | | | NRas12 | | | |
| Caki-1 | | | | | | | | | | | | | | | | | NL | | | |
| DU-145 | | | | | | | | 127 | | | | | | | | | NL | | | |
| RXF 393 | | | | | | | | | | | | | | | | | | | | |
| PC-3 | | | | | | | | 129 | | | | | | | | | | | | |
| ACHN | | | | | | | | | | | | | | | | | | | | |
| HCC-2998 | | | | | | | | | | | | | | | | | | | | |
| 786-0 | | | | | | | | | | | | | | | | | NL | | | |
| HCT 116 | | | | | | | | | | | | | | | | | | | | |
| TK-10 | | | | | | | | | | | | | | | | | | | | |
| SW480-8 | | | | | | | | | 136 | | | | | | | | KRas12 | | | |
| LOX IMVI | | | | | | | | | | | | | | | | | | | | |
| COLO 205 | | | | | | | | | | | | | | | | | NL | | | |
| Malme-3M | | | | | | | | | | | | | | | | | | | | |
| HCT-15 | | | | | | | | | | | | | | | | | KRas13 | | | |
| SK-MEL-2 | | | | | | | | | | | | | | | | | NRas61 | | | |
| KM-12 | | | | | | | | | | | | | | | | | NL | | | |
| SK-MEL-5 | | | | | | | | | | | | | | | | | | | | |
| UO-31 | | | | | | | | | | | | | | | | | | | | |
| SK-MEL-28 | | | | | | | | | | | | | | | | | NL | | | |
| UACC-62 | | | | | | | | | | | | | | | | | | | | |
| UACC-257 | | | | | | | | | | | | | | | | | NL | | | |
| M14 | | | | | | | | | | | | | | | | | | | | |
| MCF-7-1 | | | | | | | | | | | | | | | | | NL | | | |
| MCF7 | | | | | | | | | | | | | | | | | | | | |
| Hs 578T | | | | | | | | | | | | | | | | | | | | |
| MDA-MB-231 | | | | | | | | | | | | | | | | | KRas13 | | | |
| MDA-MB-435 | | | | | | | | | | | | | | | | | NL | | | |
| MDA-N | | | | | | | | | | | | | | | | | NL | | | |
| T-47D | | | | | | | | | | | | | | | | | NL | | | |
| ADR-RES-2 | | | | | | | | | | | | | | | | | | | | |
| lymph node-h | | | | | | | | | | 2 | | | | | | | | | | |
| bone marrow-h | | | | | | | | | | 3 | | | | | | | | | | |
| mammary gland-h | | | | | | | | | | | | | | | | | | | | |
| brain-h | | | | | | | | | 5 | | | | | | | | | | | |
| pancreas-h | | | | | | | | | | | | | | | | | | | | 6 |
| cerebellum-h | | | | | | | | | 7 | | | | | | | | | | | |
| pituitary gland-h | | | | | | | | | 8 | | | | | | | | | | | |
| fetal brain-h | | | | | | | | | 9 | | | | | | | | | | | |
| placenta-h | | | | | | | | | | | | | | | | 10 | | | | |
| fetal kidney-h | | | | | | | | | | | | | | | | 11 | | | | |
| prostate, h | | | | | | | | 12 | | | | | | | | | | | | |
| fetal liver-h | | | | | | | | | | | | | | | | 13 | | | | |
| Salivary gl.-h | | | | | | | | | | | | | | | | | | | | |
| fetal lung-h | | | | | | | | | | | | | | | | 15 | | | | |
| Skeletal muscle-h | | | | | | | | | | | | | | | | | | | | 16 |
| heart-h | | | | | | | | | | | | | | | | | | | | |
| small intestine-h | | | | | | | | | | | | | | | | | | | | |
| kidney-h | | | | | | | | | | | | | | | | | | | | |
| spinal cord-h | | | | | | | | | 20 | | | | | | | | | | | |
| liver-h | | | | | | | | | | | | | | | | | | | | |
| Spleen-h | | | | | | | | | | 22 | | | | | | | | | | |
| lung-h | | | | | | | | | | | | | | | | | | | | |
| stomach-h | | | | | | | | | | | | | | | | | | | | |
| testis-h | | | | | | | | | | | | | 25 | | | | | | | |
| thymus-h | | | | | | | | | | 27 | | | | | | | | | | |
| HPAEC | | | | | | | | | | | | | | | | 28 | | | | |
| thyroid gland-h | | | | | | | | | | | | | | | | | | | | |
| RPTEC | | | | | | | | | | | | | | | | 30 | | | | |
| trachea-h | | | | | | | | | | | | | | | | | | | | |
| HMEC | | | | | | | | | | | | | | | | | | | | |
| uterus-h | | | | | | | | | | | | | | | | | | | | |
| HCAEC | | | | | | | | | | | | | | | | 34 | | | | |
| pancreas-h | | | | | | | | | | | | | | | | | | | | 35 |
| lymph node-h | | | | | | | | | | 36 | | | | | | | | | | |
| Skeletal muscle-h | | | | | | | | | | | | | | | | | | | | 37 |
| fetal liver-h | | | | | | | | | | | | | | | | 38 | | | | |
| Heart-h | | | | | | | | | | | | | | | | | | | | |
| thymus, h | | | | | | | | | | 40 | | | | | | | | | | |
| Duodenum-h | | | | | | | | | | | | | | | | | | | | |
| fetal brain-h | | | | | | | | | 42 | | | | | | | | | | | |
| Salivary gl.-h | | | | | | | | | | | | | | | | | | | | |
| testis-h | | | | | | | | | | | | | 44 | | | | | | | |
| fetal kidney-h | | | | | | | | | | | | | | | | 49 | | | | |
| fetal liver-h | | | | | | | | | | | | | | | | 51 | | | | |
| fetal lung-h | | | | | | | | | | | | | | | | 53 | | | | |
| Heart-h | | | | | | | | | | | | | | | | | | | | |
| kidney-h | | | | | | | | | | | | | | | | | | | | |
| lung-h | | | | | | | | | | | | | | | | | | | | |
| lymph node-h | | | | | | | | | | 61 | | | | | | | | | | |
| WI-38 72h | | | | | | | | | | | | | | | | | | | | |
| thymus-h | | | | | | | | | | 193 | | | | | | | | | | |
| HEPM 3d TGFB1 | | | | | | | | | | | | | | | | | | | | |
| detergent + DNase | | | | | | | | | | | | | | | | | | | | |
| salivary gl.-h | | | | | | | | | | | | | | | | | | | | |
| testis-h | | | | | | | | | | | | | 199 | | | | | | | |
| Pancreas-h | | | | | | | | | | | | | | | | | | | | 201 |
| Skeletal muscle-h | | | | | | | | | | | | | | | | | | | | 203 |
| Duodenum-h | | | | | | | | | | | | | | | | | | | | |
| HMEC | | | | | | | | | | | | | | | | | | | | |
| Fetal brain-h | | | | | | | | | 210 | | | | | | | | | | | |
| HCAEC | | | | | | | | | | | | | | | | 211 | | | | |
| placenta-h | | | | | | | | | | | | | | | | 212 | | | | |
| fetal liver-h | | | | | | | | | | | | | | | | 213 | | | | |
| stomach-h | | | | | | | | | | | | | | | | | | | | |
| heart-h | | | | | | | | | | | | | | | | | | | | |
| bladder-h | | | | | | | | | | | | | | | | | | | | |
| HT372-normal | | | | | | | | | | | | | | | | | | | | |
| HT382-normal | | | | | | | | | | | | | | | | | | | | |
| HT392-normal | | | | | | | | | | | | | | | | | | | | |
| HPAEC | | | | | | | | | | | | | | | | 275 | | | | |
| ADR-RES-1 | | | | | | | | | | | | | | | | | | | | |
| bone marrow-h | | | | | | | | | | 279 | | | | | | | | | | |
| skeletal muscle-h | | | | | | | | | | | | | | | | | | | | |
| small intestine-h | | | | | | | | | | | | | | | | | | | | 290 |
| spinal cord-h | | | | | | | | | 294 | | | | | | | | | | | |
| Spleen-h | | | | | | | | | | | | | | | | | | | | |
| liver-h | | | | | | | | | | | | | | | | | | | | |
| testis-h | | | | | | | | | | | | 298 | | | | | | | | |
| bladder-h | | | | | | | | | | | | | | | | | | | | |
| mammary gland-h | | | | | | | | | | | | | | | | | | | | |
| Pancreas-h | | | | | | | | | | | | | | | | | | | | 305 |
| pituitary gland-h | | | | | | | | | | | | | | | | | | | | |
| prostate, h | | | | | | | | 309 | | | | | | | | | | | | |
| salivary gl-h | | | | | | | | | | | | | | | | | | | | |
| SA-OS (Mundy) | | | | | | | | | | | | | | | | | | | | |
| poly A+ | | | | | | | | | | | | | | | | | | | | |
| thyroid gland-h | | | | | | | | | | | | | | | | | | | | |
| trachea-h | | | | | | | | | | | | | | | | | | | | |
| uterus-h | | | | | | | | | | | | | | | | | | | | |
| HEPM 3d untreated | | | | | | | | | | | | | | | | | | | | |
| HT149-normal | | | | | | | | | | | | | | | | | | | | |
| thymus, h | | | | | | | | | | 326 | | | | | | | | | | |
| HT398-normal | | | | | | | | | | | | | | | | | | | | |
| Fetal brain-h | | | | | | | | | 328 | | | | | | | | | | | |
| h adult SMC | | | | | | | | | | | | | | | | | | | | |
| Oct. 21, 1992 #17 | | | | | | | | | | | | | | | | | | | | |
| lymph node-h | | | | | | | | | | | | | | | | | | | | |
| RPTEC | | | | | | | | | | | | | | | | | | | | |
| brain-h | | | | | | | | | 342 | | | | | | | 334 | | | | |
| cerebellum-h | | | | | | | | | 344 | | | | | | | | | | | |
| HT157-normal | | | | | | | | | | | | | | | | | | | | |
| HT213-normal | | | | | | | | | | | | | | | | | | | | |
| HT218-normal | | | | | | | | | | | | | | | | | | | | |
| HeLa-1 | CC-26 | | MC-31 | SR-21 | | | | | | | 58 | 58 | 58 | 58 | | | | | | |
| HeLa-2 | | | | SR-22 | | | | | | | 138 | | | | | | | | | |
| HeLa-3 | CC-27 | | | | | | | | | | | | 60 | | | | | | | |
| HeLa-4 | CC-28 | | | | | | | | | | | | 62 | 62 | | | | | | |
| HeLa-5 | CC-29 | | | | | | | | | | | | 64 | | | | | | | |
| HeLa-6 | | | | | | | | | | | | | | 66 | | | | | | |
| HeLa-7 | CC-30 | | MC-32 | | | | | | | | | 68 | 68 | | | | | | | |
| HeLa-8 | | | MC-33 | | | | | | | | | 70 | | | | | | | | |
| ADR-RES-3 | CC-16 | DD-28 | MC-28 | SR-19 | | | | | | | 65 | 65 | 65 | 65 | | | | | | |
| ADR-RES-4 | | | | SR-20 | | | | | | | 67 | | | | | | | | | |
| ADR-RES-5 | CC-17 | | | | | | | | | | | | 69 | | | | | | | |
| ADR-RES-6 | CC-18 | DD-29 | | | | | | | | | | | 71 | 71 | | | | | | |
| ADR-RES-7 | CC-19 | | | | | | | | | | | | 50 | | | | | | | |
| ADR-RES-8 | | DD-30 | | | | | | | | | | | | 52 | | | | | | |
| adrenal gland-h | CC-20 | | MC-29 | | | | | | | | | 54 | 54 | | | | | | | |
| adrenal gland-h | | | MC-30 | | | | | | | | | 56 | | | | | | | | |
| C33A-1 | | | MC-40 | SR-27 | | | | | | | 80 | 80 | 80 | 80 | | | | | | |
| C33A-2 | | | | SR-28 | | | | | | | 82 | | | | | | | | | |
| C33A-3 | | | | | | | | | | | | | 84 | | | | | | | |
| C33A-4 | | | | | | | | | | | | | 86 | 86 | | | | | | |
| C33A-5 | | | | | | | | | | | | | 88 | | | | | | | |
| C33A-6 | | | | | | | | | | | | | | 90 | | | | | | |
| C33A-7 | | | MC-41 | | | | | | | | | 92 | 92 | | | | | | | |
| C33A-8 | | | MC-42 | | | | | | | | | 94 | | | | | | | | |
| EKVX-1 | CC-06 | DD-04 | MC-04 | SR-03 | | | | | | | 122 | 122 | 122 | 122 | | | | | | |
| EKVX-2 | | | | SR-04 | | | | | | | 124 | | | | | | | | | |
| EKVX-3 | CC-08 | | | | | | | | | | | | 126 | | | | | | | |
| EKVX-4 | CC-07 | DD-05 | | | | | | | | | | | 128 | 128 | | | | | | 305 |
| EKVX-5 | CC-09 | | | | | | | | | | | | 17 | | | | | | | |
| EKVX-6 | | DD-06 | | | | | | | | | | | | 19 | | | | | | |
| EKVX-7 | CC-10 | | MC-05 | | | | | | | | | 21 | 21 | | | | | | | |
| EKVX-8 | | | MC-06 | | | | | | | | | 130 | | | | | | | | |
| H1299-1 | CC-31 | | MC-37 | SR-25 | | | | | | | 87 | 87 | 87 | 87 | | | | | | |
| H1299-2 | | | | SR-26 | | | | | | | 89 | | | | | | | | | |
| H1299-3 | CC-32 | | | | | | | | | | | | 91 | | | | | | | |
| H1299-4 | CC-33 | | | | | | | | | | | | 93 | 93 | | | | | | |
| H1299-5 | CC-34 | | | | | | | | | | | | 95 | | | | | | | |
| H1299-6 | | | | | | | | | | | | | | 74 | | | | | | |
| H1299-7 | CC-35 | | MC-38 | | | | | | | | | 76 | 76 | | | | | | | |
| H1299-8 | | | MC-39 | | | | | | | | | 78 | | | | | | | | |
| HT29-1 | | DD-10 | MC-10 | | | | | | | | 14 | 14 | 14 | 14 | | | | | | |
| HT29-2 | | | | SR-08 | | | | | | | 16 | | | | | | NL | | | |
| HT29-3 | | | | | | | | | | | | | 18 | | | | NL | | | |
| HT29-4 | | DD-11 | | | | | | | | | | | 20 | 20 | | | | | | |
| HT29-5 | | | | | | | | | | | | | 22 | | | | | | | |
| HT29-6 | | DD-12 | | | | | | | | | | | | 24 | | | | | | |
| HT29-7 | | | MC-11 | | | | | | | | | 25 | 25 | | | | | | | |
| HT29-8 | | | MC-12 | | | | | | | | | 27 | | | | | | | | |
| OVCAR-5-1 | | DD-22 | MC-22 | SR-15 | | | | | | | 36 | 36 | 36 | 36 | | | | | | |
| OVCAR-5-2 | | | | SR-16 | | | | | | | 38 | | | | | | | | | |
| OVCAR-5-3 | | | | | | | | | | | | | 40 | | | | | | | |
| OVCAR-5-4 | | DD-23 | | | | | | | | | | | 42 | 42 | | | | | | |
| OVCAR-5-5 | | | | | | | | | | | | | 171 | | | | | | | |
| OVCAR-5-6 | | DD-24 | | | | | | | | | | | | 46 | | | | | | |
| OVCAR-5-7 | | | MC-23 | | | | | | | | | 48 | 48 | | | | | | | |
| OVCAR-5-8 | | | MC-24 | | | | | | | | | 138 | | | | | | | | |
| SF-268-1 | | DD-16 | MC-16 | SR-11 | | | | | 120 | | 120 | 120 | 120 | 120 | | | NL | | | |
| SF-268-2 | | | | SR-12 | | | | | 121 | | 121 | | | | | | | | | |
| SF-268-3 | | | | | | | | | 123 | | | | 123 | | | | | | | |
| SF-268-4 | | DD-17 | | | | | | | 125 | | | | 125 | 125 | | | | | | |
| SF-268-5 | | | | | | | | | 127 | | | | 127 | | | | | | | |
| SF-268-6 | | DD-18 | | | | | | | 129 | | | | | 129 | | | | | | |
| SF-268-7 | | | MC-17 | | | | | | 131 | | | 131 | 131 | | | | | | | |
| SF-268-8 | | | MC-18 | | | | | | 133 | | | 133 | | | | | | | | |
| SW-620 | | | MC-34 | SR-23 | | | | | | | 72 | 72 | 72 | 72 | | | | | | |
| SW-620 | | | | SR-24 | | | | | | | 73 | | | | | | | | | |
| SW480-1 | | | | | | | | | | | | | 75 | | | | | | | |
| SW480-2 | | | | | | | | | | | | | 77 | 77 | | | | | | |
| SW480-3 | | | | | | | | | | | | | 79 | | | | | | | |
| SW480-4 | | | | | | | | | | | | | | 81 | | | | | | |
| SW480-5 | | | MC-35 | | | | | | | | | 83 | 83 | | | | | | | |
| SW480-6 | | | MC-36 | | | | | | | | | 85 | | | | | | | | |
| U2OS-1 | | | MC-43 | SR-29 | | | | | | | 96 | 96 | 96 | 96 | | | | | | |
| U2OS-2 | | | | SR-30 | | | | | | | 97 | | | | | | | | | |
| U2OS-3 | | | | | | | | | | | | | 99 | | | | | | | |
| U2OS-4 | | | | | | | | | | | | | 101 | 101 | | | | | | |
| U2OS-5 | | | | | | | | | | | | | 103 | | | | | | | |
| U2OS-6 | | | | | | | | | | | | | | 105 | | | | | | |
| U2OS-7 | | | MC-44 | | | | | | | | | 107 | 107 | | | | | | | |
| U2OS-8 | | | MC-45 | | | | | | | | | 109 | | | | | | | | |
| A549-1 | CC-01 | DD-01 | MC-01 | SR-01 | | | | | | | 1 | 1 | 1 | 1 | | | | | | |
| A549-2 | | | | SR-02 | | | | | | | 3 | | | | | | | | | |
| A549-3 | CC-02 | | | | | | | | | | | | 5 | | | | | | | |
| A549-4 | CC-03 | DD-02 | | | | | | | | | | | 7 | 7 | | | | | | |
| A549-5 | CC-04 | | | | | | | | | | | | 9 | | | | | | | |
| A549-6 | | DD-03 | | | | | | | | | | | | 11 | | | | | | |
| A549-7 | CC-05 | | MC-02 | | | | | | | | | 13 | 13 | | | | | | | |
| A549-8 | | | MC-03 | | | | | | | | | 15 | | | | | | | | |
| HCT-116-1 | | DD-07 | MC-07 | SR-05 | | | | | | | 23 | 23 | 23 | 23 | | | | | | |
| HCT-116-2 | | | | SR-06 | | | | | | | 132 | | | | | | | | | |
| HCT-116-3 | | | | | | | | | | | | | | | | | KRas13 | | | |
| HCT-116-4 | | DD-08 | | | | | | | | | | | 4 | 4 | | | | | | |
| HCT-116-5 | | | | | | | | | | | | | 6 | | | | | | | |
| HCT-116-6 | | DD-09 | | | | | | | | | | | | 8 | | | | | | |
| HCT-116-7 | | | MC-08 | | | | | | | | | 10 | 10 | | | | | | | |
| HCT-116-8 | | | MC-09 | | | | | | | | | 12 | | | | | | | | |
| Hs68-1 | | | MC-46 | SR-31 | | | | | | | 111 | 111 | 111 | 111 | | | | | | |
| Hs68-2 | | | | SR-32 | | | | | | | 113 | | | | | | | | | |
| Hs68-3 | | | | | | | | | | | | | 115 | | | | | | | |
| Hs68-4 | | | | | | | | | | | | | 117 | 117 | | | | | | |
| Hs68-6 | | | | | | | | | | | | | | 98 | | | | | | |
| Hs68-7 | | | MC-47 | | | | | | | | | 100 | 100 | | | | | | | |
| Hs68-8 | | | MC-48 | | | | | | | | | 102 | | | | | | | | |
| MCF-7-3 | CC-11 | DD-25 | MC-25 | SR-17 | | | | | | | 49 | 49 | 49 | 49 | | | | | | |
| MCF-7-4 | | | | SR-18 | | | | | | | 51 | | | | | | | | | |
| MCF-7-5 | CC-12 | | | | | | | | | | | | 53 | | | | | | | |
| MCF-7-6 | CC-13 | DD-26 | | | | | | | | | | | 55 | 55 | | | | | | |
| MCF-7-7 | CC-14 | | | | | | | | | | | | 57 | | | | | | | |
| MCF-7-8 | | DD-27 | | | | | | | | | | | | 59 | | | | | | |
| MCF-7/ADR-RES | CC-15 | | MC-26 | | | | | | | | | 61 | 61 | | | | | | | |
| MCF-7/ADR-RES | | | MC-27 | | | | | | | | | 63 | | | | | | | | |
| OVCAR-4-1 | | DD-19 | MC-19 | SR-13 | | | | | | | 45 | 45 | 45 | 45 | | | | | | |
| OVCAR-4-2 | | | | SR-14 | | | | | | | 47 | | | | | | | | | |
| OVCAR-4-3 | | | | | | | | | | | | | 26 | | | | | | | |
| OVCAR-4-4 | | DD-20 | | | | | | | | | | | 28 | 28 | | | | | | |
| OVCAR-4-5 | | | | | | | | | | | | | 30 | | | | | | | |
| OVCAR-4-6 | | DD-21 | | | | | | | | | | | | 32 | | | | | | |
| OVCAR-4-7 | | | MC-20 | | | | | | | | | 34 | 34 | | | | | | | |
| OVCAR-4-8 | | | MC-21 | | | | | | | | | 169 | | | | | | | | |
| SF539-1 | | DD-13 | MC-13 | SR-09 | | | | | 29 | | 29 | 29 | 29 | 29 | | | | | | |
| SF539-2 | | | | SR-10 | | | | | 31 | | 31 | | | | | | | | | |
| SF539-3 | | | | | | | | | 33 | | | | 33 | | | | | | | |
| SF539-4 | | DD-14 | | | | | | | 35 | | | | 35 | 35 | | | | | | |
| SF539-5 | | | | | | | | | 37 | | | | 37 | | | | | | | |
| SF539-6 | | DD-15 | | | | | | | 39 | | | | | 39 | | | | | | |
| SF539-7 | | | MC-14 | | | | | | 41 | | | 41 | 41 | | | | | | | |
| SF539-8 | | | MC-15 | | | | | | 43 | | | 43 | | | | | | | | |
| WI 38-1 | CC-21 | DD-31 | MC-49 | SR-33 | | | | | | | 104 | 104 | 104 | 104 | | | | | | |
| WI 38-2 | | | | SR-34 | | | | | | | 106 | | | | | | | | | |
| WI 38-3 | CC-22 | | | | | | | | | | | | 108 | | | | | | | |
| WI 38-4 | CC-23 | DD-32 | | | | | | | | | | | 110 | 110 | | | | | | |
| WI 38-5 | CC-24 | | | | | | | | | | | | 112 | | | | | | | |
| WI 38-6 | | DD-33 | | | | | | | | | | | | 114 | | | | | | |
| WI 38-7 | CC-25 | | MC-50 | | | | | | | | | 116 | 116 | | | | | | | |
| WI 38-8 | | | MC-51 | | | | | | | | | 118 | | | | | | | | |
| HELA-2h-031899 | | | | | | | | | | | | | 79 | | | | | | | |
| HELA-4h-031899 | | | | | | | | | | | | | 81 | | | | | | | |
| HELA-9h-031899 | | | | | | | | | | | | | 83 | | | | | | | |
| HELA-0h-031899 | | | | | | | | | | | | | 86 | | | | | | | |
| HELA-6h-031899 | | | | | | | | | | | | | 88 | | | | | | | |
| HELA-8h-031899 | | | | | | | | | | | | | 90 | | | | | | | |
| HELA-10h-031899 | | | | | | | | | | | | | 92 | | | | | | | |
| HELA-11h-031899 | | | | | | | | | | | | | 94 | | | | | | | |
| HELA-12h-031899 | | | | | | | | | | | | | 96 | | | | | | | |
| NCI-H322M | | | | | | | | | | | | | | | | | | | | |
| NCI-H460 | | | | | | | | | | | | | | | | | | | | |
| NCI-H522 | | | | | | | | | | | | | | | | | | | | |
| SNB-19 | | | | | | | | | 152 | | | | | | | | | | | |
| SNB-75 | | | | | | | | | | | | | | | | | | | | |
| SF-268 | | | | | | | | | 156 | | | | | | | | | | | |
| SF-295 | | | | | | | | | 158 | | | | | | | | | | | |
| CCRF-CEM | | | | | | | | | | 160 | | | | | | | | | | |
| DU-145 | | | | | | | | | 162 | | | | | | | | NL | | | |
| HCT 116 | | | | | | | | | | | | | | | | | | | | |
| Cacki-1 | | | | | | | | | | | | | | | | | | | | |
| 786-0 | | | | | | | | | | | | | | | | | NL | | | |
| T-47D | | | | | | | | | | | | | | | | | | | | |
| CRL1441 RNA 8/30 | | | | | | | | | | | | | | | | | | | | |
| 781T untreeated + | | | | | | | | | | | | | | | | | | | | |
| DNase | | | | | | | | | | | | | | | | | | | | |
| KB poly A+ | | | | | | | | | | | | | | | | | | | | |
| HOS poly A+ | | | | | | | | | | | | | | | | | | | | |
| ACHN | | | | | | | | | | | | | | | | | NL | | | |
| UACC-62 | | | | | | | | | | | | | | | | | NL | | | |
| MCF7 | | | | | | | | | | | | | | | | | NL | | | |
| UTOS (Mundy) | | | | | | | | | | | | | | | | | | | | |
| poly a+ | | | | | | | | | | | | | | | | | | | | |
| WISH (Collagen) | | | | | | | | | | | | | | | | | | | | |
| poly A+ | | | | | | | | | | | | | | | | | | | | |
| 458 medullo mRNA | | | | | | | | | | | | | | | | | | | | |
| CCL137 RNA | | | | | | | | | | | | | | | | | | | | |
| Mar. 21, 1988 | | | | | | | | | | | | | | | | | | | | |
| WI-38 72 h 0.5% | | | | | | | | | | | | | | | | | | | | |
| FBS, 24 h 10% FBS | | | | | | | | | | | | | | | | | | | | |
| CRL 1441 + TPA | | | | | | | | | | | | | | | | | | | | |
| (24h) 8/30 | | | | | | | | | | | | | | | | | | | | |
| HOP-92 | | | | | | | | | | | | | | | | | | | | |
| MOLT-4 | | | | | | | | | | 242 | | | | | | | NRas12- | | | |
| EKVX | | | | | | | | | | | | | | | | | NL | | | |
| HL-60 | | | | | | | | | | 244 | | | | | | | NRas61 | | | |
| NCI-H23 | | | | | | | | | | | | | | | | | NRas12 | | | |
| RPMI 8226 | | | | | | | | | | 246 | | | | | | | | | | |
| A549/ATCC | | | | | | | | | | | | | | | | | KRas12 | | | |
| SR | | | | | | | | | | 248 | | | | | | | | | | |
| OVCAR-3 | | | | | | | | | | | | | | | | | NL | | | |
| HCT-15 | | | | | | | | | | | | | | | | | | | | |
| OVCAR-4 | | | | | | | | | | | | | | | | | NL | | | |
| UO-31 | | | | | | | | | | | | | | | | | NL | | | |
| OVCAR-5 | | | | | | | | | | | | | | | | | | | | |
| SN12C | | | | | | | | | | | | | | | | | | | | |
| OVCAR-8 | | | | | | | | | | | | | | | | | NL | | | |
| LOX IMVI | | | | | | | | | | | | | | | | | NL | | | |
| IGROV1 | | | | | | | | | | | | | | | | | | | | |
| SK-MEL-2 | | | | | | | | | | | | | | | | | | | | |
| SK-OV-3 | | | | | | | | | | | | | | | | | NL | | | |
| SK-MEL-5 | | | | | | | | | | | | | | | | | NL | | | |
| SF-539 | | | | | | | | | | | | | | | | | | | | |
| SK-MEL-28 | | | | | | | | | | | | | | | | | | | | |
| K-562 | | | | | | | | | | 263 | | | | | | | | | | |
| UACC-257 | | | | | | | | | | | | | | | | | | | | |
| M14 | | | | | | | | | | | | | | | | | NL | | | |
| MCF-7-2 | | | | | | | | | | | | | | | | | | | | |
| MDA-MB-435 | | | | | | | | | | | | | | | | | | | | |
| HT279 | | | | | | | | | | | | | | | | | | | | |
| MDA-N | | | | | | | | | | | | | | | | | | | | |
| Y79 poly A+ | | | | | | | | | | | | | | | | | | | | |
| KHOS poly A+ | | | | | | | | | | | | | | | | | | | | |
| HTB36 24 h TPA | | | | | | | | | | | | | | | | | | | | |
| RNA 6/23 | | | | | | | | | | | | | | | | | | | | |
| HELA-EXP-031899 | | | | | | | | | | | | | 313 | | | | | | | |
| HTB36 0h RNA | | | | | | | | | | | | | | | | | | | | |
| HT347 | | | | | | | | | | | | | | | | | | | | |
| 458 medullo RNA | | | | | | | | | 324 | | | | | | | | | | | |
| NCI-H226 | | | | | | | | | | | | | | | | | NL | | | |
| HOP-62 | | | | | | | | | | | | | | | | | KRas12 | | | |
| MDA-MB-231 | | | | | | | | | | | | | | | | | | | | |
| U251 | | | | | | | | | | | | | | | | | | | | |
| PT cells poly A+ | | | | | | | | | | | | | | | | | | | | |
| PC-3 | | | | | | | | 341 | | | | | | | | | NL | | | |
| HCC-2998 | | | | | | | | | | | | | | | | | NL | | | |
| SW480-7 | | | | | | | | | | | | | | | | | | | | |
| HT192 | | | | | | | | | | | | | | | | | | | | |
| COLO 205 | | | | | | | | | | | | | | | | | | | | |
| HT218 | | | | | | | | | | | | | | | | | | | | |
| KM-12 | | | | | | | | | | | | | | | | | | | | |
| HT151 | | | | | | | | | | | | | | | | | | | | |
| A498 | | | | | | | | | | | | | | | | | | | | |
| HT393 | | | | | | | | | | | | | | | | | | | | |
| RXF 393 | | | | | | | | | | | | | | | | | NL | | | |
| TK-10 | | | | | | | | | | | | | | | | | NL | | | |
| Malme-3M | | | | | | | | | | | | | | | | | NL | | | |
| Hs 578T | | | | | | | | | | | | | | | | | HRas12 | | | |
| HT213 | | | | | | | | | | | | | | | | | | | | |
| HT288 | | | | | | | | | | | | | | | | | | | | |
| HT139 | | | | | | | | | | | | | | | | | | | | |
| HT155 | | | | | | | | | | | | | | | | | | | | |
| HT163 | | | | | | | | | | | | | | | | | | | | |
| HT170 | | | | | | | | | | | | | | | | | | | | |
| HT172 | | | | | | | | | | | | | | | | | | | | |
| HT138 | | | | | | | | | | | | | | | | | | | | |
| HT178 | | | | | | | | | | | | | | | | | | | | |
| HT154 | | | | | | | | | | | | | | | | | | | | |
| HT180 | | | | | | | | | | | | | | | | | | | | |
| HT169 | | | | | | | | | | | | | | | | | | | | |
| HT189 | | | | | | | | | | | | | | | | | | | | |
| HT143 | | | | | | | | | | | | | | | | | | | | |
| HT190 | | | | | | | | | | | | | | | | | | | | |
| HT145 | | | | | | | | | | | | | | | | | | | | |
| HT227 | | | | | | | | | | | | | | | | | | | | |
| HT302 | | | | | | | | | | | | | | | | | | | | |
| HT314 | | | | | | | | | | | | | | | | | | | | |
| HT317 | | | | | | | | | | | | | | | | | | | | |
| Medulloblastoma | | | | | | | | | | 77 | | | | | | | | | | |
| #425 11/8 | | | | | | | | | | | | | | | | | | | | |
| HT323 | | | | | | | | | | | | | | | | | | | | |
| HT327 | | | | | | | | | | | | | | | | | | | | |
| HT335 | | | | | | | | | | | | | | | | | | | | |
| HT146 | | | | | | | | | | | | | | | | | | | | |
| HT348 | | | | | | | | | | | | | | | | | | | | |
| HT311 | | | | | | | | | | | | | | | | | | | | |
| HT398 | | | | | | | | | | | | | | | | | | | | |
| HT140 | | | | | | | | | | | | | | | | | | | | |
| HT281 | | | | | | | | | | | | | | | | | | | | |
| HT372 | | | | | | | | | | | | | | | | | | | | |
| TCGP | | | | | | | | | | | | | | | | | | | | |
| HT160 | | | | | | | | | | | | | 207 | | | | | | | |
| HT307 | | | | | | | | | | | | | | | | | | | | |
| HT369 | | | | | | | | | | | | | | | | | | | | |
| HT370 | | | | | | | | | | | | | | | | | | | | |
| HT371 | | | | | | | | | | | | | | | | | | | | |
| HT377 | | | | | | | | | | | | | | | | | | | | |
| HT382 | | | | | | | | | | | | | | | | | | | | |
| neruoblastoma RNA | | | | | | | | | 281 | | | | | | | | | | | |
| HT334 | | | | | | | | | | | | | | | | | | | | |
| HT338 | | | | | | | | | | | | | | | | | | | | |
| HT392 | | | | | | | | | | | | | | | | | | | | |
| HT394 | | | | | | | | | | | | | | | | | | | | |
| HT312 | | | | | | | | | | | | | | | | | | | | |
| HT162 | | | | | | | | | | | | | | | | | | | | |
| HT395 | | | | | | | | | | | | | | | | | | | | |
| HT157 | | | | | | | | | | | | | | | | | | | | |
| 458 medullo RNA | | | | | | | | | | | | | | | | | | | | |
| CRL1572 | | | | | | | | | | | | | | | | | | | | |
| Mar. 17, 1989 | | | | | | | | | | | | | | | | | | | | |
| h fibroblasts | | | | | | | | | | | | | | | | | | | | |
| Mar. 31, 1992 #12 | | | | | | | | | | | | | | | | | | | | |
| h keratinocytes | | | | | | | | | | | | | | | | | | | | |
| Feb. 25, 1992 #10 | | | | | | | | | | | | | | | | | | | | |
| HT308 | | | | | | | | | | | | | | | | | | | | |
| HT368 | | | | | | | | | | | | | | | | | | | | |
| HT378 | | | | | | | | | | | | | | | | | | | | |
| HT385 | | | | | | | | | | | | | | | | | | | | |

Table 7 gives results of a PCR screen of 48 human cDNA sources for 26 of the kinases represented in this application. A plus sign (+) indicates the presence of a band on an agarose gel of the expected size for the target kinase. A negative sign (−) indicates that the PCR product of the expected size was absent. The genes represented on this table are: SEQ ID NO:6, SEQ ID NO:7, SEQ ID NO:8, SEQ ID NO:10, SEQ ID NO:12, SEQ ID NO:13, SEQ ID NO:14, SEQ ID NO:16, SEQ ID NO:22, SEQ ID NO:23, SEQ ID NO:24, SEQ ID NO:26, SEQ ID NO:28, SEQ ID NO:31, SEQ ID NO:33, SEQ ID NO:39, SEQ ID NO:40, SEQ ID NO:43, SEQ ID NO:45, SEQ ID NO:46, SEQ ID NO:47, SEQ ID NO:48, SEQ ID NO:49, SEQ ID NO:51, SEQ ID NO:52 and SEQ ID NO:56.

TABLE
PCR Expression Analysis

| | | | | | SGK069 | SGK110 | SGK053 | SGK254 |
|---|---|---|---|---|---|---|---|---|
| Tissue_name | gel-well | 96-well pos | RNA_source | Tissue/Cells | NA #6 | NA #7 | NA #8 | NA #10 |
| fetal liver-h | 1 | A01 | Clontech | Normal tissue | − | − | − | − |
| thymus, h | 2 | A02 | Clontech | Normal tissue | − | − | − | − |
| pancreas-h | 3 | A03 | Clontech | Normal tissue | − | − | − | − |
| pituitary gland-h | 4 | A04 | Clontech | Normal tissue | − | − | − | − |
| placenta-h | 5 | A05 | Clontech | Normal tissue | − | + | − | − |
| prostate, h | 6 | A06 | Clontech | Normal tissue | − | − | − | − |
| salivary gl.-h | 7 | A07 | Clontech | Normal tissue | − | − | − | − |
| skeletal muscle-h | 8 | A08 | Clontech | Normal tissue | + | − | − | − |
| small intestine-h | 9 | A09 | Clontech | Normal tissue | − | − | − | − |
| spinal cord-h | 10 | A10 | Clontech | Normal tissue | − | − | − | − |
| Spleen-h | 11 | A11 | Clontech | Normal tissue | − | + | − | − |
| stomach-h | 12 | A12 | Clontech | Normal tissue | − | − | + | − |
| thyroid gland-h | 13 | B01 | Clontech | Normal tissue | − | + | − | − |
| trachea-h | 14 | B02 | Clontech | Normal tissue | − | − | − | − |
| uterus-h | 15 | B03 | Clontech | Normal tissue | − | + | − | − |
| adrenal gland-h | 16 | B04 | Clontech | Normal tissue | − | − | − | − |
| fetal brain-h | 17 | B05 | Clontech | Normal tissue | − | − | − | + |
| fetal kidney-h | 18 | B06 | Clontech | Normal tissue | − | − | + | − |
| fetal lung-h | 19 | B07 | Clontech | Normal tissue | − | − | + | − |
| heart-h | 20 | B08 | Clontech | Normal tissue | − | − | + | − |
| kidney-h | 21 | B09 | Clontech | Normal tissue | − | − | − | − |
| liver-h | 22 | B10 | Clontech | Normal tissue | − | − | − | − |
| lung-h | 23 | B11 | Clontech | Normal tissue | − | + | + | − |
| lymph node-h | 24 | B12 | Clontech | Normal tissue | − | − | + | − |
| Heart-h | 25 | C01 | Sugen | Normal tissue | − | − | − | − |
| HPAEC - Renal proximal | 26 | C02 | Sugen | Normal Cell line | − | − | − | − |
| tubule epithelial cells | | | | | | | | |
| RPTEC - Mammary | 27 | C03 | Sugen | Normal Cell line | − | + | − | − |
| epithelial cells | | | | | | | | |
| HMEC - Coronary artery | 28 | C04 | Sugen | Normal Cell line | − | − | − | − |
| endothelial cells | | | | | | | | |
| HCAEC - Coronary artery | 29 | C05 | Sugen | Normal Cell line | − | − | − | − |
| endothelial cells | | | | | | | | |
| 458 medullo RNA - | 30 | C06 | Sugen | Tumor | − | − | − | − |
| Neuroblastoma | | | | | | | | |
| A549/ATCC - Lung | 31 | C07 | NCI | Cancer Cell line | − | − | − | − |
| carcinoma | | | | | | | | |
| MDA-MB-231 - | 32 | C08 | NCI | Cancer Cell line | − | + | − | − |
| Brest adenocarcinoma, | | | | | | | | |
| pleural effusion | | | | | | | | |
| Hs 578T - Ductal | 33 | C09 | NCI | Cancer Cell line | − | + | − | − |
| breastcarcinoma | | | | | | | | |
| MCF-7/ADR-RES - | 34 | C10 | NCI | Cancer Cell line | − | − | − | + |
| Breast adenocarcinoma | | | | | | | | |
| Malme-3M - Malignant | 35 | C11 | NCI | Cancer Cell line | − | + | − | + |
| melanoma, metastasis to | | | | | | | | |
| lung | | | | | | | | |
| A498 - Kidney carcinoma | 36 | C12 | NCI | Cancer Cell line | + | + | − | − |
| COLO 205 - Colon | 37 | D01 | NCI | Cancer Cell line | − | + | − | − |
| adenocarcinoma | | | | | | | | |
| CCRF-CEM - ALL Acute | 38 | D02 | NCI | Cancer Cell line | − | + | − | − |
| lymphobllastic leukemia | | | | | | | | |
| SF-539 - Glioblastoma | 39 | D03 | NCI | Cancer Cell line | − | + | − | − |
| SF-295 - Glioblastoma | 40 | D04 | NCI | Cancer Cell line | − | − | + | − |
| U251 - Glioblastoma | 41 | D05 | NCI | Cancer Cell line | − | + | + | − |
| SNB-19 - Glioblastoma | 42 | D06 | NCI | Cancer Cell line | − | − | − | − |
| OVCAR-4 - Ovary | 43 | D07 | NCI | Cancer Cell line | − | − | − | + |
| adenocarcinoma | | | | | | | | |
| OVCAR-3 - Ovary | 44 | D08 | NCI | Cancer Cell line | − | + | − | − |
| adenocarcinoma | | | | | | | | |
| TCGP - Tesicular | 45 | D09 | GP | Tumor | − | − | − | − |
| carcinoma | | | | | | | | |
| HMEC - Coronary artery | 46 | D10 | GP | Normal Cell line | − | − | − | − |
| endothelial cells | | | | | | | | |
| HOP-62 - Lung | 47 | D11 | NCI | Cancer Cell line | − | − | − | − |
| adenocarcinoma | | | | | | | | |
| NCI-H522 - Lung | 48 | D12 | NCI | Cancer Cell line | − | − | − | − |
| adenocarcinoma | | | | | | | | |

| | SGK411 | SGK027 | SGK046b | SGK089 | SGK003 | SGK066 | SGK041 | SGK038 | SGK429 |
|---|---|---|---|---|---|---|---|---|---|
| Tissue_name | NA #12 | NA #13 | NA #14 | NA #16 | NA #22 | NA #23 | NA #24 | NA #26 | NA #28 |
| fetal liver-h | − | − | − | − | − | − | − | − | + |
| thymus, h | − | − | − | − | − | − | − | − | − |
| pancreas-h | − | − | − | − | − | − | − | − | − |
| pituitary gland-h | − | + | − | − | − | − | − | − | − |
| placenta-h | − | − | + | − | − | − | − | + | + |
| prostate, h | − | − | + | − | − | − | + | − | − |
| salivary gl.-h | − | − | + | − | − | + | − | + | − |
| skeletal muscle-h | − | − | + | − | − | + | − | − | − |
| small intestine-h | − | + | + | − | − | − | − | − | − |
| spinal cord-h | + | + | + | − | + | + | − | − | + |
| Spleen-h | − | − | − | − | − | − | − | − | − |
| stomach-h | − | − | + | − | − | + | − | − | − |
| thyroid gland-h | − | + | − | + | − | − | + | − | + |
| trachea-h | − | + | − | + | − | − | + | + | − |
| uterus-h | − | − | − | + | + | + | + | + | − |
| adrenal gland-h | − | + | − | − | − | + | − | − | − |
| fetal brain-h | + | + | − | − | + | + | − | + | − |
| fetal kidney-h | + | − | − | − | + | − | − | + | − |
| fetal lung-h | − | − | + | − | + | − | − | − | − |
| heart-h | − | − | − | − | − | − | − | + | − |
| kidney-h | + | − | + | + | − | + | − | − | − |
| liver-h | − | − | − | − | − | − | − | − | − |
| lung-h | + | + | + | − | − | + | − | − | − |
| lymph node-h | − | + | + | + | − | + | + | − | − |
| Heart-h | − | − | − | − | − | + | − | − | − |
| HPAEC - Renal proximal | − | − | − | − | − | − | − | − | − |
| tubule epithelial cells | | | | | | | | | |
| RPTEC - Mammary | − | − | − | − | − | + | − | − | + |
| epithelial cells | | | | | | | | | |
| HMEC - Coronary artery | − | − | + | − | − | − | − | − | + |
| endothelial cells | | | | | | | | | |
| HCAEC - Coronary artery | − | − | − | − | − | − | − | − | − |
| endothelial cells | | | | | | | | | |
| 458 medullo RNA - | − | − | + | − | − | + | − | − | − |
| Neuroblastoma | | | | | | | | | |
| A549/ATCC - Lung | + | − | + | − | + | − | + | + | + |
| carcinoma | | | | | | | | | |
| MDA-MB-231 - | − | + | + | + | + | − | − | + | + |
| Brest adenocarcinoma, | | | | | | | | | |
| pleural effusion | | | | | | | | | |
| Hs 578T - Ductal | − | − | + | + | + | − | − | − | + |
| breastcarcinoma | | | | | | | | | |
| MCF-7/ADR-RES - | − | + | − | − | + | − | − | − | − |
| Breast adenocarcinoma | | | | | | | | | |
| Malme-3M - Malignant | − | − | + | − | + | − | − | − | + |
| melanoma, metastasis to | | | | | | | | | |
| lung | | | | | | | | | |
| A498 - Kidney carcinoma | − | − | − | − | − | − | + | − | − |
| COLO 205 - Colon | − | − | + | − | + | − | + | − | − |
| adenocarcinoma | | | | | | | | | |
| CCRF-CEM - ALL Acute | − | + | + | − | + | + | + | − | − |
| lymphobllastic leukemia | | | | | | | | | |
| SF-539 - Glioblastoma | − | + | + | + | + | + | − | − | − |
| SF-295 - Glioblastoma | − | + | + | + | + | + | + | − | − |
| U251 - Glioblastoma | + | + | + | + | + | − | − | − | − |
| SNB-19 - Glioblastoma | − | + | + | + | + | − | − | − | − |
| OVCAR-4 - Ovary | + | − | + | + | − | + | + | − | − |
| adenocarcinoma | | | | | | | | | |
| OVCAR-3 - Ovary | − | − | + | + | − | + | − | − | − |
| adenocarcinoma | | | | | | | | | |
| TCGP - Tesicular | − | − | − | + | + | − | − | − | − |
| carcinoma | | | | | | | | | |
| HMEC - Coronary artery | − | − | − | − | + | + | − | − | − |
| endothelial cells | | | | | | | | | |
| HOP-62 - Lung | − | − | + | − | − | − | + | − | − |
| adenocarcinoma | | | | | | | | | |
| NCI-H522 - Lung | + | − | + | − | − | + | − | − | − |
| adenocarcinoma | | | | | | | | | |

| | | | | | SGK093 | SGK087 | SGK009 | SGK421 |
|---|---|---|---|---|---|---|---|---|
| Tissue₋ name | gel-well | 96-well pos | RNA₋ source | Tissue/Cells | NA #31 | NA #33 | NA #39 | NA #40 |
| fetal liver-h | 1 | A01 | Clontech | Normal tissue | − | − | − | − |
| thymus, h | 2 | A02 | Clontech | Normal tissue | − | − | − | − |
| pancreas-h | 3 | A03 | Clontech | Normal tissue | − | − | − | − |
| pituitary gland-h | 4 | A04 | Clontech | Normal tissue | − | − | − | − |
| placenta-h | 5 | A05 | Clontech | Normal tissue | + | − | − | − |
| prostate, h | 6 | A06 | Clontech | Normal tissue | + | − | + | − |
| salivary gl.-h | 7 | A07 | Clontech | Normal tissue | + | + | + | − |
| skeletal muscle-h | 8 | A08 | Clontech | Normal tissue | + | − | − | − |
| small intestine-h | 9 | A09 | Clontech | Normal tissue | + | + | − | − |
| spinal cord-h | 10 | A10 | Clontech | Normal tissue | + | − | − | + |
| Spleen-h | 11 | A11 | Clontech | Normal tissue | + | + | − | − |
| stomach-h | 12 | A12 | Clontech | Normal tissue | + | − | + | − |
| thyroid gland-h | 13 | B01 | Clontech | Normal tissue | + | + | + | − |
| trachea-h | 14 | B02 | Clontech | Normal tissue | + | − | + | − |
| uterus-h | 15 | B03 | Clontech | Normal tissue | − | − | − | − |
| adrenal gland-h | 16 | B04 | Clontech | Normal tissue | + | + | − | − |
| fetal brain-h | 17 | B05 | Clontech | Normal tissue | + | − | − | + |
| fetal kidney-h | 18 | B06 | Clontech | Normal tissue | + | + | + | + |
| fetal lung-h | 19 | B07 | Clontech | Normal tissue | + | + | + | − |
| heart-h | 20 | B08 | Clontech | Normal tissue | − | − | − | − |
| kidney-h | 21 | B09 | Clontech | Normal tissue | + | − | + | − |
| liver-h | 22 | B10 | Clontech | Normal tissue | − | − | − | − |
| lung-h | 23 | B11 | Clontech | Normal tissue | + | + | + | − |
| lymph node-h | 24 | B12 | Clontech | Normal tissue | + | + | − | − |
| Heart-h | 25 | C01 | Sugen | Normal tissue | − | − | − | − |
| HPAEC - Renal proximal | 26 | C02 | Sugen | Normal Cell line | − | − | − | − |
| tubule epithelial cells | | | | | | | | |
| RPTEC - Mammary | 27 | C03 | Sugen | Normal Cell line | + | − | − | − |
| epithelial cells | | | | | | | | |
| HMEC - Coronary artery | 28 | C04 | Sugen | Normal Cell line | + | − | − | − |
| endothelial cells | | | | | | | | |
| HCAEC - Coronary artery | 29 | C05 | Sugen | Normal Cell line | + | − | − | − |
| endothelial cells | | | | | | | | |
| 458 medullo RNA - | 30 | C06 | Sugen | Tumor | + | − | − | − |
| Neuroblastoma | | | | | | | | |
| A549/ATCC - Lung | 31 | C07 | NCI | Cancer Cell line | + | − | − | − |
| carcinoma | | | | | | | | |
| MDA-MB-231 - | 32 | C08 | NCI | Cancer Cell line | + | − | − | − |
| Brest adenocarcinoma, | | | | | | | | |
| pleural effusion | | | | | | | | |
| Hs 578T - Ductal | 33 | C09 | NCI | Cancer Cell line | + | − | − | − |
| breastcarcinoma | | | | | | | | |
| MCF-7/ADR-RES - | 34 | C10 | NCI | Cancer Cell line | + | − | − | − |
| Breast adenocarcinoma | | | | | | | | |
| Malme-3M - Malignant | 35 | C11 | NCI | Cancer Cell line | + | − | + | − |
| melanoma, metastasis to | | | | | | | | |
| lung | | | | | | | | |
| A498 - Kidney carcinoma | 36 | C12 | NCI | Cancer Cell line | + | − | − | − |
| COLO 205 - Colon | 37 | D01 | NCI | Cancer Cell line | + | + | + | − |
| adenocarcinoma | | | | | | | | |
| CCRF-CEM - ALL Acute | 38 | D02 | NCI | Cancer Cell line | + | + | − | − |
| lymphobllastic leukemia | | | | | | | | |
| SF-539 - Glioblastoma | 39 | D03 | NCI | Cancer Cell line | − | − | − | − |
| SF-295 - Glioblastoma | 40 | D04 | NCI | Cancer Cell line | + | + | − | − |
| U251 - Glioblastoma | 41 | D05 | NCI | Cancer Cell line | + | − | + | − |
| SNB-19 - Glioblastoma | 42 | D06 | NCI | Cancer Cell line | + | − | − | − |
| OVCAR-4 - Ovary | 43 | D07 | NCI | Cancer Cell line | + | + | + | − |
| adenocarcinoma | | | | | | | | |
| OVCAR-3 - Ovary | 44 | D08 | NCI | Cancer Cell line | + | − | − | − |
| adenocarcinoma | | | | | | | | |
| TCGP - Tesicular | 45 | D09 | GP | Tumor | + | + | − | − |
| carcinoma | | | | | | | | |
| HMEC - Coronary artery | 46 | D10 | GP | Normal Cell line | + | − | + | − |
| endothelial cells | | | | | | | | |
| HOP-62 - Lung | 47 | D11 | NCI | Cancer Cell line | + | + | − | − |
| adenocarcinoma | | | | | | | | |
| NCI-H522 - Lung | 48 | D12 | NCI | Cancer Cell line | + | − | + | − |
| adenocarcinoma | | | | | | | | |

| | SGK396 | SGK037 | SGK060 | SGK080 | SGK002 | SGK058 | SGK035 | SGK075 | SGK007 |
|---|---|---|---|---|---|---|---|---|---|
| Tissue₋ name | NA #43 | NA #45 | NA #46 | NA #47 | NA #48 | NA #49 | NA #51 | NA #52 | NA #56 |
| fetal liver-h | − | − | − | − | − | − | + | − | − |
| thymus, h | − | − | − | − | − | − | − | − | − |
| pancreas-h | − | − | − | − | − | − | − | + | − |
| pituitary gland-h | − | − | + | − | − | − | + | − | − |
| placenta-h | − | − | − | − | − | − | + | + | − |
| prostate, h | − | − | − | + | − | + | + | − | − |
| salivary gl.-h | − | − | + | − | − | − | − | − | − |
| skeletal muscle-h | − | − | + | − | − | − | + | − | − |
| small intestine-h | − | − | − | − | − | − | + | − | − |
| spinal cord-h | − | + | + | − | − | + | + | − | − |
| Spleen-h | − | − | − | − | − | − | + | − | − |
| stomach-h | − | − | + | + | − | − | + | + | − |
| thyroid gland-h | − | − | + | − | − | − | + | − | − |
| trachea-h | − | + | + | − | − | + | + | − | − |
| uterus-h | − | − | − | − | − | − | + | − | − |
| adrenal gland-h | + | − | − | − | − | − | − | − | − |
| fetal brain-h | + | − | − | − | − | + | + | − | − |
| fetal kidney-h | − | − | − | − | − | − | + | − | − |
| fetal lung-h | − | + | + | − | − | + | + | − | − |
| heart-h | − | − | − | − | − | − | + | − | − |
| kidney-h | − | − | − | − | − | − | + | − | − |
| liver-h | − | − | − | − | − | − | + | − | − |
| lung-h | − | + | + | − | − | + | − | − | − |
| lymph node-h | − | − | + | + | − | − | + | + | − |
| Heart-h | − | − | − | − | − | − | − | − | − |
| HPAEC - Renal proximal | − | − | − | − | − | − | + | − | − |
| tubule epithelial cells | | | | | | | | | |
| RPTEC - Mammary | − | − | − | − | − | − | + | − | − |
| epithelial cells | | | | | | | | | |
| HMEC - Coronary artery | − | − | − | − | − | − | + | + | − |
| endothelial cells | | | | | | | | | |
| HCAEC - Coronary artery | − | − | − | − | − | − | + | − | − |
| endothelial cells | | | | | | | | | |
| 458 medullo RNA - | − | − | − | − | − | − | + | − | − |
| Neuroblastoma | | | | | | | | | |
| A549/ATCC - Lung | − | − | + | − | − | − | + | + | − |
| carcinoma | | | | | | | | | |
| MDA-MB-231 - | − | − | − | + | − | − | + | + | − |
| Brest adenocarcinoma, | | | | | | | | | |
| pleural effusion | | | | | | | | | |
| Hs 578T - Ductal | − | − | − | + | − | − | + | + | − |
| breastcarcinoma | | | | | | | | | |
| MCF-7/ADR-RES - | − | − | − | + | − | − | + | + | − |
| Breast adenocarcinoma | | | | | | | | | |
| Malme-3M - Malignant | − | − | − | + | − | − | + | − | − |
| melanoma, metastasis to | | | | | | | | | |
| lung | | | | | | | | | |
| A498 - Kidney carcinoma | − | − | − | + | − | − | + | + | − |
| COLO 205 - Colon | − | + | − | + | − | − | + | + | − |
| adenocarcinoma | | | | | | | | | |
| CCRF-CEM - ALL Acute | − | − | + | + | + | − | + | + | − |
| lymphobllastic leukemia | | | | | | | | | |
| SF-539 - Glioblastoma | − | − | + | + | + | − | + | + | − |
| SF-295 - Glioblastoma | − | − | − | + | + | − | + | − | − |
| U251 - Glioblastoma | − | − | + | + | + | − | + | + | − |
| SNB-19 - Glioblastoma | − | − | + | + | + | − | + | − | − |
| OVCAR-4 - Ovary | − | − | − | + | − | − | + | + | − |
| adenocarcinoma | | | | | | | | | |
| OVCAR-3 - Ovary | − | − | + | + | − | − | + | + | − |
| adenocarcinoma | | | | | | | | | |
| TCGP - Tesicular | − | − | − | + | − | − | + | − | − |
| carcinoma | | | | | | | | | |
| HMEC - Coronary artery | − | − | − | + | − | − | + | − | − |
| endothelial cells | | | | | | | | | |
| HOP-62 - Lung | + | − | − | + | − | − | + | + | − |
| adenocarcinoma | | | | | | | | | |
| NCI-H522 - Lung | − | − | − | + | − | − | + | − | − |
| adenocarcinoma | | | | | | | | | |

Table 8 contains results of probing a multiple tissue expression blot from Clontech (http://www.clontech.com/) with two of the genes from this application, SGK093 (SEQ ID NO:31); and SGK138, (SEQ ID NO:53).

TABLE
Clontech Multiple Tissue Blot

| | SGK093 | SGK188 |
|---|---|---|
| Clontech Tissue Blot | ID#NA_31 | ID#NA_53 |
| accumbens nucleus | 365 | 3092 |
| adrenal gland | 64 | 570 |
| amygdala | 309 | 4796 |
| aorta | 360 | 309 |
| apex of the heart | 305 | 517 |
| appendix | 677 | 1695 |
| atrium, left | 480 | 446 |
| atrium, right | 561 | 232 |
| bladder | 32 | 539 |
| bone marrow | 408 | 421 |
| Burkitt's lymphoma, Daudi | 399 | 0 |
| Burkitt's lymphoma, Raji | 430 | 206 |
| caudate nucleus | 187 | 8750 |
| cerebellum left | 541 | 1960 |
| cerebellum, right | 680 | 1436 |
| cerebral cortex | 126 | 6185 |
| colon, ascending | 3480 | 7453 |
| colon, descending | 11042 | 3958 |
| colon, transverse | 4419 | 4555 |
| colorectal adeno-carcinoma, SW480 | 1379 | 1369 |
| corpus callosum | 142 | 2221 |
| duodenum | 4014 | 2200 |
| esophagus | 3973 | 612 |
| fetal brain | 321 | 5202 |
| fetal heart | 627 | 709 |
| fetal kidney | 12081 | 199 |
| fetal liver | 9488 | 424 |
| fetal lung | 448 | 334 |
| fetal spleen | 210 | 110 |
| fetal thymus | 225 | 361 |
| frontal lobe | 432 | 3891 |
| heart | 285 | 394 |
| HeLa S3 | 500 | 390 |
| hippocampus | 552 | 5100 |
| ileum | 2598 | 2072 |
| ilocecum | 1307 | 3820 |
| inter-ventricular septum | 0 | 100 |
| jujenum | 2588 | 858 |
| kidney | 15961 | 152 |
| leukemia, HL-60 | 879 | 242 |
| leukemia, K582 | 14760 | 246 |
| leukemia, MOLT-4 | 201 | 344 |
| liver | 494 | 649 |
| lung | 928 | 710 |
| lung carcinoma, A549 | 815 | 1439 |
| lymph node | 1044 | 1503 |
| mamary gland | 1659 | 1139 |
| medulla oblongata | 360 | 3260 |
| occipital lobe | 199 | 7343 |
| ovary | 1056 | 275 |
| pancreas | 856 | 1696 |
| paracentral gyrus cerebral cortex | 195 | 4394 |
| parietal lobe | 219 | 6799 |
| peripheral blood leukocyte | 19 | 342 |
| pituitary gland | 2009 | 4355 |
| placenta | 284 | 543 |
| pons | 0 | 4160 |
| prostate | 10296 | 984 |
| putamen | 85 | 5033 |
| rectum | 11181 | 4398 |
| salivary gland | 281 | 1232 |
| skeletal muscle | 156 | 214 |
| spinal cord | 895 | 1084 |
| spleen | 124 | 603 |
| stomach | 1941 | 2124 |
| substantia nigra | 332 | 2418 |
| temperal lobe | 0 | 7253 |
| testis | 607 | 31121 |
| thalamus | 502 | 2902 |
| thymus | 162 | 127 |
| thyroid gland | 128 | 24 |
| trachea | 1393 | 926 |
| uterus | 420 | 443 |
| ventricle, left | 502 | 563 |
| ventricle, right | 268 | 859 |
| whole brain | 256 | 5291 |

### EXAMPLES

The examples below are not limiting and are merely representative of various aspects and features of the present invention. The examples below demonstrate the isolation and characterization of the nucleic acid molecules according to the invention, as well as the polypeptides they encode.

### Example 1

### Identification and Characterization of Genomic Fragments Encoding Protein Kinases

Materials and Methods

Novel kinases were identified from the Celera human genomic sequence databases, and from the public Human Genome Sequencing project (http://www.ncbi.nlm.nih.gov/) using a hidden Markov model (HMMR) built with 70 mammalian and yeast kinase catalytic domain sequences. These sequences were chosen from a comprehensive collection of kinases such that no two sequences had more than 50% sequence identity. The genomic database entries were translated in six open reading frames and searched against the model using a Timelogic Decypher box with a Field programmable array (FPGA) accelerated version of HMMR2.1. The DNA sequences encoding the predicted protein sequences aligning to the HMMR profile were extracted from the original genomic database. The nucleic acid sequences were then clustered using the Pangea Clustering tool to eliminated repetitive entries. The putative protein kinase sequences were then sequentially run through a series of queries and filters to identify novel protein kinase sequences. Specifically, the HMMR identified sequences were searched using BLASTN and BLASTX against a nucleotide and amino acid repository containing 634 known human protein kinases and all subsequent new protein kinase sequences as they are identified. The output was parsed into a spreadsheet to facilitate elimination of known genes by manual inspection. Two models were developed, a “complete” model and a “partial” or Smith Waterman model. The partial model was used to identify sub-catalytic kinase domains, whereas the complete model was used to identify complete catalytic domains. The selected hits were then queried using BLASTN against the public nrna and EST databases to confirm they are indeed unique. In some cases the novel genes were judged to be homologues of previously identified rodent or vertebrate protein kinases.

Extension of partial DNA sequences to encompass the full-length open-reading frame was carried out by several methods. Iterative blastn searching of the cDNA databases listed in Table 9 was used to find cDNAs that extended the genomic sequences. “LifeSeqGold” databases are from Incyte Genomics, Inc (http://www.incyte.com/). NCBI databases are from the National Center for Biotechnology Information (http://www.ncbi.nlm.nih.gov/). All blastn searches were conducted using a penalty for a nucleotide mismatch of −3 and reward for a nucleotide match of 1. The gapped blast algorithm is described in: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

Extension of partial DNA sequences to encompass the full-length open-reading frame was also carried out by iterative searches of genomic databases. The first method made use of the Smith-Waterman algorithm to carry out protein-protein searches of a close protein homologue to the partial. The target databases consisted of Genscan and open-reading frame (ORF) predictions of all human genomic sequence derived from the human genome project (HGP) as well as from Celera. The complete set of genomic databases searched is shown in Table 10, below. Genomic sequences encoding potential extensions were further assessed by blastx analysis against the NCBI nonredundant database to confirm the novelty of the hit. The extending genomic sequences were incorporated into the cDNA sequence after removal of potential introns using the Seqman program from DNAStar. The default parameters used for Smith-Waterman searches were as shown next. Matrix: blosum 62; gap-opening penalty: 12; gap extension penalty: 2. Genscan predictions were made using the Genscan program as detailed in Chris Burge and Sam Karlin “Prediction of Complete Gene Structures in Human Genomic DNA”, JMB (1997) 268(1):78-94). ORF predictions from genomic DNA were made using a standard 6-frame translation.

Another method for defining DNA extensions from genomic sequence used iterative searches of genomic databases through the Genscan program to predict exon splicing. These predicted genes were then assessed to see if they represented “real” extensions of the partial genes based on homology to related kinases.

Another method involved using the Genewise program (http://www.sanger.ac.uk/Software/Wise2/ ) to predict potential ORFs based on homology or HMM to the closest homologue. Genewise requires two inputs, the homologous protein, and genomic DNA containing the gene of interest. The genomic DNA was identified by blastn searches of Celera and Human Genome Project databases. The homologues were identified by blastp searches of the NCBI non-redundant protein database (NRAA) with the predicted protein sequence derived from the HMM search of the genomic database. Genewise compares the protein sequence to a genomic DNA sequence, allowing for introns and frameshifting errors.

TABLE 9
Databases used for cDNA-based sequence extensions

| Database | Database Date |
|---|---|
| LifeGold templates | October 2000 |
| LifeGold compseqs | October 2000 |
| LifeGold compseqs | October 2000 |
| LifeGold compseqs | October 2000 |
| LifeGold fl | October 2000 |
| LifeGold flft | October 2000 |
| NCBI human Ests | October 2000 |
| NCBI murine Ests | October 2000 |
| NCBI nonredundant | October 2000 |

Database Number of entries Database Date

TABLE 10
Databases used for genomic-based sequence extensions

| | Number of | Database |
|---|---|---|
| Database | entries | Date |
| Celera v. 1-5 | 5,306,158 | Jan 19/00 |
| Celera v. 6-10 | 4,209,980 | Mar 24/00 |
| Celera v. 11-14 | 7,222,425 | Apr 24/00 |
| Celera v. 15 | 243,044 | May 14/00 |
| Celera v. 16-17 | 25,885 | Apr 04/00 |
| Celera Assembly 5 (R1.25) | 3,313 | Oct 13/00 |
| Celera Assembly 4 (R1.24) | 636,234 | Aug 28/00 |
| Celera Assembly 3 (R 1.22, | 1,132,900 | Jul 21/00 |
| 1.23) | | |
| HGP Phase 0 | 4,944 | May 04/00 |
| HGP Phase 1 | 28,478 | May 05/00 |
| HGP Phase 2 | 1,508 | May 04/00 |
| HGP Phase 3 | 9,971 | May 05/00 |
| HGP Phase 0 | 3,189 | Nov 1/00 |
| HGP Phase 1 | 20,447 | Nov 1/00 |
| HGP Phase 2 | 1,619 | Nov 1/00 |
| HGP Phase 3 | 9,224 | Nov 1/00 |
| HGP Chromosomal | 2759 | Aug 1/00 |
| assemblies | | |

Results:

The sources for the sequence information used to extend the genes in the provisional patents are listed below. For genes that were extended using Genewise, the accession numbers of the protein homologue and the genomic DNA are given. (Genewise uses the homologue to assemble the coding sequence of the target gene from the genomic sequence). The amino acid sequences for the homologs were obtained from the NCBI non-redundant database of proteins (http://www.ncbi.nlm.nih.gov/Entrez/protein.html). The genomic DNA came from two sources: Celera and NCBI-NRNA, as indicated below. cDNA sources are also listed below. Abbreviations: HGP: Human Genome Project; NCBI, National Center for Biotechnology Information.

SGK187, ID#NA_{—}1, ENCODING SEQ ID NO:58
Gene name: CRIK
Genewise homologs: AAC72823.1, AAC25483.1,
Genomic DNA sources:
Celera 181000000994928; 17000140635257
NCBI AC002563.1, BAC clone 277F10, AC004813

SGK064, ID#NA_{—}2, ENCODING SEQ ID NO:59
Gene name: GRK7 G protein-coupled receptor kinase,
Genewise homolog: AAC9500, gi_{—}4001826
Genomic DNA sources: Celera, 90000626316497, public hgp contig gi|8 1 3 9 7 1 6
Celera 11000284009826, 17000062696662

Incyte cDNA: 7477204CB1 (99% identical)
Compared with the homolog, the Celera genewise predicted only the N-terminus; the hgp contig was phase 1 and so composed of unordered pieces. It's genewise had two predictions, the second of which extended the prediction to the end of the homolog. A single AA (M) was added to the beginning, based on a Genscan prediction on the Celera contig.

SGK409 (SEQ ID NO:3, ENCODING SEQ ID NO:60)
Genewise homologs: NP_{—}032667, BAA76817
FLv predicted from contig of SGK409 (at 5′ end to nucleotide 2193) and KIAA0303 (at 3′ end, from 2194 to 77850)

SGK021 (SEQ ID NO:4, ENCODING SEQ ID NO:61)
Genewise homologs: CAB76566, BAA95027, CAB76471, NP_{—}060871
Genomic DNA sources:

Celera 11000283991970, 17000028181153, 17000077758366, 173000019633053, 173000019394610; Incyte cDNA: 1110037.1
Notes: ESTs confirm part of the sequence, but essentially this is a genewise prediction on 90000641092679 using gi_{—}7161864

SGK410 (SEQ ID NO:5, ENCODING SEQ ID NO:62)
Genewise homolog: NP_{—}002731 (PKC, iota_h)
Genomic DNA source: AL133280.12
Note: FL from Genewise prediction from HGP contig AL133280.12 using NP_{—}002731.1

SGK069 (SEQ ID NO:6, ENCODING SEQ ID NO:63)
Genewise homolog: BAA36362
Genomic DNA sources: HGP_{—}7191033_{—}7, 11000284155330
Celera: 90000641726632

SGK110 (SEQ ID NO:7, ENCODING SEQ ID NO:64)
Genewise homolog: BAA36362
Genomioc sources:
HGP_{—}7191033_{—}7
Celera: 11000283338966, 17000140258105, 17000077607693, 11000283338966 and 17000077607693

SGK053, ID#NA_{—}8, ENCODING SEQ ID NO:65)
Gene name: CKLiK
Genewise homolog: Q63450, NM_{—}020397.1
Genomic DNA sources: 17000035790284
cDNAs from dbEST: BE266955, A1923704, AW501047

SGK124 (SEQ ID NO:9, ENCODING SEQ ID NO:66)
Genewise homolog: gi_{—}4827159
Genomic DNA source: 90000641832427
Celera: 17000047891899,
NCBI: dJ1103G7.3
cDNA: Incyte 328225.13

SGK254 (SEQ ID NO:10, ENCODING SEQ ID NO:67)
Genewise homolog: AAB46910
Genomic DNA source: 17000091533743, AC005940

SGK297, ID#NA_{—}11, ENCODING SEQ ID NO:68)
Gene name: CaMKIb2_h
Genomic DNA source:
Celera 17000140614482_{—}2, 17000113122540
cDNA sources: Incyte 827431CB1; dbEST R87839

SGK411 (SEQ ID NO:12, ENCODING SEQ ID NO:69)
Genewise homolog: AAD20442
Genomic DNA sources: HGP 2828765_{—}1_{—}3
cDNA sources: NCBI AF071569

SGK027 (SEQ ID NO: 13, ENCODING SEQ ID NO:70)
Genewise homologs: AAF69801, AAA97437
FL cloned from adipose and brain
Genomic DNA sources:
Celera 17000057443791, 17000029868654,CA2_GS_N_{—}106000011351167_{—}1
CA2_GS_N_{—}106000011351167_{—}1, 17000057443791 on N-term
cDNA: Incyte 321074.1
SGK027 has been cloned as a full length gene from human brain and adipocyte libraries.

SGK046b (SEQ ID NO:14, ENCODING SEQ ID NO:71)
Genewise homologs: AAC33487, AAF69801
Genomic DNA sources: Celera 11000283376057, 11000283376057

SGK046c (SEQ ID NO:15, ENCODING SEQ ID NO:72)
Genewise homologs: AAC33487 AAF69801
Genomic DNA sources: Celera 11000284253087, 11000284253087

SGK089 (SEQ ID NO:16, ENCODING SEQ ID NO:73)
Genewise homologs: AAF64455, AAA97437
Genomic DNA sources: Celera 11000283986586
cDNA source: NCBI AK024110

SGK133, ID#NA_{—}17, ENCODING SEQ ID NO:74)
Genewise homolog: CAA07196
Genomic DNA sources: 17000075929111, 17000048133019, 17000076096636, 10 17000140484696
cDNA sources: NCBI N83965; Incyte: 999618, 7477486CB1, 984011.1

SGK004 (SEQ ID NO: 18, ENCODING SEQ ID NO:75
Genewise homolog: gi|9978891
Gene name: MSK, SIK (AB020480, salt-inducible protein kinase),
Genomic DNA sources: Celera 17000084574278, 17000048604376, 78000006706941

SGK006 (SEQ ID NO:19, ENCODING SEQ ID NO:76)
Genewise homolog: NP_{—}056570, gi|7657216.
Genomic DNA sources: Celera 11000283913252, 78000005691234

SGK180, ID#NA_{—}20, ENCODING SEQ ID NO:77)
Gene name: SNRK_h
Genewise homolog: gi|7303211
Genomic DNA sources: Celera 17000057577785, 181000001006215

cDNA sources: NCBI AF226044, AK000231 (N-term), D43636 KIAA0096 (C-term)

SGK386, ID#NA_{—}21, ENCODING SEQ ID NO:78)
Gene name: MLCKs_h
Genomic DNA sources:
Celera 17000140249749_{—}2, 17000140438265
HGP: 7242443_{—}3, AL160175, AL160175.5

SGK003 (SEQ ID NO:22, ENCODING SEQ ID NO:79)
Genewise homolog: P70065, gi|6166236
Genomic DNA sources:
Celera 17000083956390, 17000062613386, 17000036890480, 780000063066170, 78000006306170
FL genewise prediction from Celera sequence using CKIalpha P70065 as a model; manually corrected by addition of seq gataactaa at 1006-1014 based on pairwise comparison between aa seq for SGK003 and CKIalpha_h NP_{—}001883.2 and CKI, epsilon_h A57011. Ctermimus of SGK003 confirmed with HGP AL391383.4. Genscan predicts a similar protein from the same contig, with a stop two AAs after the end of the genewise prediction, so those two AAs (DN) were added to the genewise prediction, which then made it the same length as it's closest public homolog.

SGK066 (SEQ ID NO:23, ENCODING SEQ ID NO:80)
Genewise homolog: T42260
Genomic DNA source: 11000283296349
cDNA source: dbEST AA234451

SGK041 (SEQ ID NO:24, ENCODING SEQ ID NO:81)
Gene name: NKIAMRE
Genomic DNA sources: Celera 17000062743907, 17000076002106, 17000048152347, 17000091635260
cDNA sources: AF130372
Genewise was carried out on Celera contig 300702668 using protein homologs gi_{—}4505569, gi_{—}7001374, and gi_{—}7706059. Genscan predicts an extension to the C-terminus, which extends the homology to a rat homolog further. The last 4 AA of the genewise prediction disagree with the genscan prediction and were removed. The Genscan prediction is supported by NCBI ESTs: gi|10823329, gi|2963750, gi|511720. All lose homology with the genscan at the same point, where we believe the genscan prediction to be incorrect. The C-terminal end of the gene was predicted using these EST sequences along with Celera genomic sequence, and the open reading frame extended down to the first verified stop.
Genewise shows that this gene has 12 exons, as follows (co-ordinates are from the predicted cDNA; the kinase domain is 4-286)
1-165, 166-360, 361-539, 540-652, 653-792, 793-881,-882-1035, 1036-1364, 1365-1458, 1459-1621, 1622-1719, 1720-1773.
Many ESTs align to the gene, but are missing one or more exons, showing a large degree of alternative splicing in this gene
3943561T8 and 3943561F8 read through the intron between exons 7 and 8. This is unlikely to be functional, as it shifts the frame and encodes stop codons, but might result in a truncated protein.
Public EST gi|2849359 is missing exon 3
Incyte EST 6553714H1 is misisng exon 5
Incyte EST 6442763H1 is missing exon 6
Incyte EST 918059R1 is missing exon 7
Incyte ESTs 1436805F1, 1436805H1, 1436805F6, 1436805F1 and 1436805H1, and public ESTs gi|677041 and gi|776279 are missing exons 5 and 6
Public EST gi|1521237 is missing exons 5-7
Incyte EST 6859179H1 and public EST gi|5368476 are missing exons 6 and 7
Public EST gi|4988790 is missing exons 6-9
Public EST gi|2963750 is missing exons 7 & 8

The public NKIAMRE sequence of this gene, and many ESTs read through the end of exon 8 into intron 8 and encode a shorter protein form. IncyteESTs 2494301F6 and 2494301H1 continue from exon4 into intron 4 before splicing out to exon5, and so introduce some extra sequence into the protein

| | | |
|---|---|---|
| 1,bp = n | TLAAPGDIYTDYVATRWYRAPELVLKDTSYGK------------PVDIWALGCMIIEMAT | (SEQ ID NO: 117) |
| | :::::::::::::::::::::::::::::::: :::::::::::.. | |
| 1,bp = n | TLAAPGDIYTDYVATRWYRAPELVLKDTSYGKYVYFGILSAFPRPVDIWALGCMIMRWPL | (SEQ ID NO: 118) |

SGK112 (SEQ ID NO:25, ENCODING SEQ ID NO:82)
Genewise homolog: S22745, gi_{—}107655
Genomic DNA source: Celera 17000035915087, 300960782
cDNA sources: Incyte 1698381 CB1
Comparison with incyte EST 1698381CB1 gives a C-terminal extension, confirmed with further genewise. The contig was assembled and extended by the addition of the following ESTs: 1698381CB1, 058298.1, 5314910H1, 1698381T6, 1698381F6, and 2539246. The contig was confirmed by comparison to genomic sequences in Celera contigs 173000022366173, 173000022366176, 173000022366174, and 300968782.

SGK038 (SEQ ID NO:26, ENCODING SEQ ID NO:83)
Gene name: ERK7
Genewise homolog: AAD12719
Genomic DNA sources: 17000030278391, 17000057882051, 17000084204744, 11000283886869,
cDNA sources: Incyte 253132.48 gave start Met; Incyte 2613981F6 gave stop.
Genewise with 142000016404854 and gi_{—}4220888. Comparison with NCBI EST gi|9510335 adds on 3AA at the end to bring it to a stop, and incyte EST adds a short N-terminal extension. The internal sequence (SEQ ID NO: 119) “DMGFLLAPPTHTPVFLSLQ” is not predicted by genewise, and is not seen in another EST (incyte 253132.50) and appears to be an alternative splice form.

SGK158 (SEQ ID NO:27, ENCODING SEQ ID NO:84)
Genewise homologs: Q60936 Q92338
CDNA source: Incy406057_{—}19
Genewise with hgp contig gi|9797056 and homologs gi|3025215, gi|7292815. The result is identical in part to a number of hypothetical proteins from human and mouse. Incyte EST 406057.19 extends and confirms the full length sequence.

SGK429 (SEQ ID NO:28, ENCODING SEQ ID NO:85)
Genewise homolog: CAB76039
Genomic DNA sources: HGP_{—}5001549_{—}3;
cDNA sources: Incyte 052560.1
Notes: Genewise with 90000642611957 and homologs gi|5441947, gi|7106068, gi|7331802. Prediction is extended and verified by NCBI ESTs: gi|10326317, gi|10159451, gi|8278227, gi|9096664, gi|9096207, gi|570679, gi|10223903.

SGK152 (SEQ ID NO:29, ENCODING SEQ ID NO:86)
Gene name: SUDD
Genomic DNA source: 11000258249295
Genewise with 90000641287353 and gi|7295659, extended with NCBI ESTs gi|10952245, gi|10991510, gi|10998012, gi|9892217, gi|9772905.
SGK077 (SEQ ID NO:30, ENCODING SEQ ID NO:87)
Genewise homolog: NP_{—}034483, gi_{—}7106329
Genomic DNA sources: HGP BAC_AF168787; Celera 11000283672012, 66000026756418 17000057552303
CDNA sources: Incyte 068072.1

Note: When checked against EST database, a short stretch of the genewise prediction was shown not to match several ESTs (incyte template 068072.1, LGf1 387382CB1 and LGf1ft sequence 387382CB1). The polymorphism occurs in the middle of an exon. The polymorphic variants differ in the following region.

| (SEQ ID NO: 120) | | |
|---|---|---|
| SGK077_1 | RPPQKCSTPCGPLRLPPFPSRDSGRLSPDLSVCGQPRDGDELGISASLFSSLASPCPGSP | 60 |
| | RPPQKCSTP P P+ LSPDLSVCGQPRDGDELGISASLFSSLASPCPGSP | |
| (SEQ ID NO: 121) | | |
| SGK077_2 | RPPQKCSTPAARSDFRPSPAAIRP-LSPDLSVCGQPRDGDELGISASLFSSLASPCPGSP | 901081 |

SGK093, Wnk3 (SEQ ID NO:31, ENCODING SEQ ID NO:88)
Genewise homolog: AAF74258
Genomic DNA sources: Celera 11000284063441, 11000284266180; HGP
CA2_GS_N_{—}111000004311351_{—}2
Note: FLv from Genscan prediction from Celera 96000001680843; corrected manually by deletion of aa sequence (SEQ ID NO: 122)
“HRLGECWQKMRRRQQGAAGGNFPVGGSFPEDVSPHQDSGYAPSPR” which did not fit the HMM prediction and did not align to NEK7. Genscan prediction supported by multiple, overlapping cDNA sequences (Incyte 8036923J1, cluster 77056_{—}1 Inc, NCI CGAP EST BF000369.1, Incyte 7934590H1, Incyte 604878F6 and cluster 806401_{—}1). The translated sequence was compared to the Genscan database of gene predictions from Celera Assembly release 3, and was found to match a predicted coding sequence from contig 96000001680843. Comparison of the predicted structure with other members of this family led to a deletion of predicted amino acids 253-297 in the putative catalytic domain. The upstream sequence was confirmed and extended with EST sequences 7459014H1, 2683093F6, 1001757.2, and 6789813F8. Two additional exons with a total of 177 nucleotides were added at amino acid position 655 (of the original Genscan prediction) based on ESTs 1001757.2 and 6789813F8. The sequences between amino acid 739 and 770 of the original prediction were replaced by a section of 697 nucleotides, based on ESTs 6622825J1, 715385, 7605034J1, 197338H1, 7609905H1, 7613564H1, 3734294F6, and 5290816T6. The remainder of the sequence was confirmed and extended with by the addition of EST 475276.9, 3038391H1, and 2963959.

SGK074 (SEQ ID NO:32, ENCODING SEQ ID NO:89)
Genewise homolog: NP_{—}034562
Genomic DNA sources:
Celera 11000283476699, 17000113567391;
HGP 6758860_{—}1_{—}5

SGK087 (SEQ ID NO:33, ENCODING SEQ ID NO:90)
Genewise homologs: AAD47290, NP_{—}003574, gi_{—}4503427, gi_{—}5702386
Genomic DNA sources: Celera 82000011582439
Celera 11000283914919, 17000036171996,
HGP BAC_AC005832

SGK295, KIS (SEQ ID NO:34, ENCODING SEQ ID NO:91)
Genewise homolog: NP_{—}058989 (KIS_r), X98374
Genomic DNA sources: Celera 17000113079883

SGK419 (SEQ ID NO:35, ENCODING SEQ ID NO:92)
Genewise homologs: CAB70863, NP_{—}055726
Genomic DNA sources: HGP_{—}1 DKFZp434P0116_h
CDNA sources: NM_{—}017593
Notes: the contig is 92000005058101 and the genewise template is gi_{—}6807781.

SGK125, MYO3A (SEQ ID NO:36, ENCODING SEQ ID NO:93)
Gene name: MYO3A
Genomic DNA sources: Celera 17000047912903, 11000502322294, 17000078090910, 17000077958351, 39000026222925,
CDNA source: NM_{—}017433.1

SGK445 (SEQ ID NO:37, ENCODING SEQ ID NO:94)
Genewise homolog: NP_{—}055079
Genomic DNA source:
Celera: 59000028993040, 4000001803382,
NCBI: 5420628

SGK127 (SEQ ID NO:38, ENCODING SEQ ID NO:95)
Genewise homolog: NP_{—}038599
Genomic DNA sources: Celera 17000084323304, 17000047957940, 17000140021687, 96000001217832_{—}1, 17000062804843
CDNA sources: Incyte 1041923.1, 7474604CB1
Genewise with Celera contig and homolog 90000642658207 and homolog gi|7305215, gave a partial prediction. A region similar to the unmatched part of the homolog could be genewised with the same homolog from 90000642658125. Incyte EST 7474604CB1 confirmed the full clone.

SGK009, ANKRD3 (SEQ ID NO:39, ENCODING SEQ ID NO:96)
Genewise homolog: gi_{—}7768736, gi_{—}7768736
Genomic DNA source:AP001743 (Homo sapiens genomic DNA, chromosome 21q),

AP001743.1, contig 78000006822431. Splice variant:

| | | |
|---|---|---|
| 1,bp = n | RPELPPVCRARPRACSHLIRLMQRCWQGDPRVRPTFQ----------------------- | |
| | ::::::::::::::::::::::::::::::::::::: | |
| SGK009 | RPELPPVCRARPRACSHLIRLMQRCWQGDPRVRPTFQGNGLNGELIRQVLAALLPVTGRW | |
| | 250 260 270 280 290 300 | |
| | 280 290 300 310 | |
| 1,bp = n | -------------------------EITSETEDLCEKPDDEVKETAHDLDVKSPPEPRSE | (SEQ ID NO: 123) |
| | ::::::::::::::::::::::::::::::::::: | |
| SGK009 | RSPGEGFRLESEVIIRVTCPLSSPQEITSETEDLCEKPDDEVKETAHDLDVKSPPEPRSE | (SEQ ID NO: 124) |

SGK421 (SEQ ID NO:40, ENCODING SEQ ID NO:97)
Gene name: STK22A, TSK1
Genewise homolog: NP_{—}033461
Genomic DNA sources: Celera 4000001803622;

SGK047 (SEQ ID NO:41, ENCODING SEQ ID NO:98)
Genewise homolog: NP_{—}004320
Genomic DNA source: Celera 11000258262374

SGK196 (SEQ ID NO:42, ENCODING SEQ ID NO:99)
Genewise homologs: T01289, AA210451
Genomic DNA source: 17000062537825
cDNA source: AK027009.1

SGK396 (SEQ ID NO:43, ENCODING SEQ ID NO: 100)
Genewise homologs: CAB90410, CAA11152
Genomic DNA sources:
HGP: 5630059_{—}1_{—}4, AL048858, HGP_{—}1_{—}5630059_{—}1_{—}4
Celera: 17000091618909, 173000013978058

SGK279, ID#NA_{—}44, ENCODING SEQ ID NO:101)
Gene Name: PKN
Genewise homologs: BAA36362, S71887
Genomic DNA sources: Celera 11000508234504, 17000091439256, 17000097259742, 17000084154777
CDNA sources: Incyte 7312543CB1

SGK037, ID#NA_{—}45, ENCODING SEQ ID NO: 102)
Genewise homolog: P51956, P51954
Genomic DNA sources: 17000036048987, 17000030265658, 17000062680964.

Note: FL virtual from genewise/Genscan of Celera assembly 173000036274838; “MDK” start from HGP 6996171 (positions 144536-114544). Cterm predicted from Genscan of Celera assembly 173000036274838.

SGK060 (SEQ ID NO:46, ENCODING SEQ ID NO:103)
Genewise homolog: NP_{—}035979, gi_{—}6754822
Genomic DNA sources: Celera 17000036897142, 84000006837210
Genewise gave a short prediction, which was extended with ESTs: NCBI gi|9334037, gi|1690632, gi|1471842, gi|4196791, gi|8149570 and Incyte sequence 7477585CB1. Incyte sequence 281154.4 shows an alternative splice form; missing (SEQ ID NO: 125)
“SLACILYEMCCMNHAFAGSNFLSIVLKIVEGDTPSLPERYPKELNAIMESMLNKNPSLRPSAIEILKIPYLDEQLQNLMCRYSEM TLEDKNLDCQKEAAHIINAM” from the sequence at ˜648-962 on the predicted cDNA.

SGK080 (SEQ ID NO:47, ENCODING SEQ ID NO:104)
Genewise homolog: NP_{—}002488
Genomic DNA sources:
Celera: 17000048119006, 11000283866229, 17000084037705, 17000084478382, 17000084564941, 17000096840584, 17000062653550, 17000112945690,
HGP: BAC_AP000532=genomic DNA, chromosome 22q11.2, Cat Eye Syndrome region

SGK002 (SEQ ID NO:48, ENCODING SEQ ID NO:105)
Genewise homolog: P36507
Genomic DNA sources: 11000501691092,
Note: FL from Genewise prediction from HGP AC018639.8 using MPK2_h P36507 as a template.

SGK058 (SEQ ID NO:49, ENCODING SEQ ID NO: 106)
Genewise homologs: A48084, AAC97114
Genomic DNA sources:
Celera 17000036225371, 111000004304440
HGP: AK026727.1
cDNA source: Incyte 217301.4

SGK103 (SEQ ID NO:50, ENCODING SEQ ID NO:107)
Genewise homolog: CAA39285, AAA28552
Genomic DNA source: Celera 11000284272557

SGK035 (SEQ ID NO:51, ENCODING SEQ ID NO:108)
Genewise homolog: Q13177
Genomic DNA sources: Celera 17000030169905, 39000025586693,39000025680595, 92000003842663
HGP: gi_{—}3041712

SGK075 (SEQ ID NO:52, ENCODING SEQ ID NO:109)
Genewise homologs: NP-059129 P50527
Genomic DNA sources: Celera 11000283492249, 11000284453752, 39000025994824, 17000113802883

Notes: Genewise with gi_{—}10440888 and 301266624, and 334000009836768 with gi_{—}10440888, along with NCBI EST gi|8362435, followed by Genewise on 301266624 using a composite prediction as template. The incyte EST is missing the bolded sequence. An alternative splice was noted with the fallowing sequence inserted after bp 933 sequence: ccctatgctgaacagagggactacaaaggcaaatcagatgcagttcctgataaagagctaatagtctgg (SEQ ID NO: 126). This insert maintains the same reading frame, with the inset coding for the following peptide sequence: PYAEQRDYKGKSDAVPDKELIVW (SEQ ID NO: 127). This inseert occurs after amino acid 311 (i.e., after the R in the following sequence:

(SEQ ID NO: 128)

KHQNPVAKTR[PYAEQRDYKGKSDAVPDKELIVW]).

SGK188, (SEQ ID NO:53, ENCODING SEQ ID NO:110))
Gene name: EphA9
Genomic DNA sources: 17000057739161, 17000065041969, 17000062798377;
cDNA sources: Incyte 7474721CB1

SGK040 (SEQ ID NO:54, ENCODING SEQ ID NO: 111)
Genewise homologs: P41243, T33475, gi_{—}729890 and gi_{—}7508561
Genomic DNA sources: Celera 11000257912897; HGP 11000257912897, contig 173000019399041.
Genewise prediction was extended by ESTs and an open reading frame identified. The ESTs were: NCBI sequences gi|10722519, gi|1920438, gi|1927810, gi|8656982, gi|4609396, gi|6087575 and Incyte sequences 215217.8, 215217.7, 1242491CB1

SGK390 (SEQ ID NO:55, ENCODING SEQ ID NO: 112)
Genewise homolog: Q64398
Genomic DNA sources: 17000077925509, 17000077861414
CDNA sources: 7474637CB1

SGK007 (SEQ ID NO:56, ENCODING SEQ ID NO: 113)
Genewise homolog: T42260
Genomic DNA sources: Celera 17000113227249

SGK050 (SEQ ID NO:57, ENCODING SEQ ID NO:114)
Genewise homolog: P16067
Genomic DNA source: Celera 17000035767558

SGK187, CRIK (SEQ ID NO:1, ENCODING SEQ ID NO:58) is 6159 nucleotides long. The open reading frame starts at position 1 and ends at position 6159, giving a ORF length of 6159 nucleotides. The predicted protein is 2053 amino acids long. This sequence is full length (start methionine to stop codon). It is classified as (superfamily/group/family): protein kinase, AGC, DMPK. This gene maps to chromosomal position 12q24.23. Amplification of this chromosomal position has been associated with the following human diseases: Non-small cell lung cancer (12q24.1-24.3; 2/50) (Knuutila, et al.). This gene contains candidate single nucleotide polymorphisms at the following positions: 2874=R (ss1337340); 2883=Y (rs904655); 3327=R (ss1581624). ESTs for this gene in the public domain (dbEST) are: BE909486, BE875297, AW605350. This gene has repetitive sequence at the following nucleotide positions: none.

SGK064, GRK7 (SEQ ID NO:2, ENCODING SEQ ID NO:59) is 1662 nucleotides long. The open reading frame starts at position 1 and ends at position 1659, giving a ORF length of 1659 nucleotides. The predicted protein is 553 amino acids long. This sequence is full length (start methionine to stop codon).It is classified as (superfamily/group/family): protein kinase, AGC, GRK. This gene maps to chromosomal position 3q24. This chromosomal position has been associated with the following human diseases: squamous cell carcinomas of the head and neck (3/30) and Uterine cervix cancer (3/10), (Knuutila, et al.), Usher syndrome (OMIM, 276902 USHER SYNDROME, TYPE III; USH3). This gene contains candidate single nucleotide polymorphisms at the following positions: 965=K; 1318=R. ESTs for this gene in the public domain (dbEST) are: none. This gene has repetitive sequence at the following nucleotide positions: 295 to 314.

SGK409, KIAA0303 (SEQ ID NO:3, ENCODING SEQ ID NO:60) is 7785 nucleotides long. The open reading frame starts at position 1 and ends at position 7569, giving a ORF length of 7569 nucleotides. The predicted protein is 2523 amino acids long. This sequence is full length (start methionine to stop codon).It is classified as (superfamily/group/family): protein kinase, AGC, MAST. This gene maps to chromosomal position 5q12.1. This chromosomal position has been associated with the following human diseases: cancer of the testis (15q15-qter; 2/11) (Knuutila, et al.). This gene contains candidate single nucleotide polymorphisms at the following positions: 6282=M ; 6327=M. ESTs for this gene in the public domain (dbEST) are: BE515326, BE267294, AA926642. This gene has repetitive sequence at the following nucleotide positions: 2355 to 2375.

SGK021 (SEQ ID NO:4, ENCODING SEQ ID NO:61) is 981 nucleotides long. The open reading frame starts at position 1 and ends at position 981, giving a ORF length of 981 nucleotides. The predicted protein is 327 amino acids long. This sequence contains the entire catalytic region of a novel kinase. It is classified as (superfamily/group/family): protein kinase, AGC, Mo3C11.1_ce. This gene maps to chromosomal position 5q31.2. This chromosomal position has been associated with the following human diseases: Chondrosarcoma (2/45), (Knuutila, et al.). This gene contains candidate single nucleotide polymorphisms at the following positions: 9=S; 97=R. ESTs for this gene in the public domain (dbEST) are: none. This gene has repetitive sequence at the following nucleotide positions: none.

SGK410 (SEQ ID NO:5, ENCODING SEQ ID NO:62) is 2260 nucleotides long. The open reading frame starts at position 72 and ends at position 1964, giving a ORF length of 1893 nucleotides. The predicted protein is 631 amino acids long. This sequence is full length (start methionine to stop codon).It is classified as (superfamily/group/family): protein kinase, AGC, PKC. This gene maps to chromosomal position Xq23. This chromosomal position has been associated with the following human diseases: cancer of the prostate (Xq23-qter; 1/9), (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK069 (SEQ ID NO:6, ENCODING SEQ ID NO:63) is 1716 nucleotides long. The open reading frame starts at position 1 and ends at position 1716, giving a ORF length of 1716 nucleotides. The predicted protein is 572 amino acids long. This sequence contains the entire catalytic region of a novel kinase. It is classified as (superfamily/group/family): protein kinase, AGC, Unique. This gene maps to chromosomal position 19pl 1-p13. This chromosomal position has been associated with the following human diseases: Small cell lung cancer (19p12, 2/22) (Knuutila, et al.). This gene contains candidate single nucleotide polymorphisms at the following positions: 1180=S (ss1317629); 210=Y (ss1688813). ESTs for this gene in the public domain (dbEST) are: none. This gene has repetitive sequence at the following nucleotide positions: 1656-1678.

SGK110 (SEQ ID NO:7, ENCODING SEQ ID NO:64) is 1119 nucleotides long. The open reading frame starts at position 1 and ends at position 1119, giving a ORF length of 1119 nucleotides. The predicted protein is 373 amino acids long. This sequence is a partial kinase catalytic domain. It is classified as (superfamily/group/family): protein kinase, AGC, Unique. This gene maps to chromosomal position 19q13.4. This chromosomal position has been associated with the following human diseases: cancer of the breast (19q13.1-qter; 1/33), (Knuutila, et al.). This gene contains candidate single nucleotide polymorphisms at the following positions: 597=R (rs654439); 252=Y (ss661406). ESTs for this gene in the public domain (dbEST) are: none. This gene has repetitive sequence at the following nucleotide positions: none.

SGK053, CKLiK (SEQ ID NO:8, ENCODING SEQ ID NO:65) is 1074 nucleotides long. The open reading frame starts at position 1 and ends at position 1071, giving a ORF length of 1071 nucleotides. The predicted protein is 357 amino acids long. This sequence is full length (start methionine to stop codon).It is classified as (superfamily/group/family): protein kinase, CAMK, AMPK. This gene maps to chromosomal position 10p14. This chromosomal position has been associated with arrhythmogenic right ventricular dysplasia (OMIM, 604401 ARRHYTHMOGENIC RIGHT VENTRICULAR DYSPLASIA, FAMILIAL). This gene contains candidate single nucleotide polymorphisms at the following positions: 605=Y; 509=M. ESTs for this gene in the public domain (dbEST) are: BE266955, A1923704, AW501047. This gene has repetitive sequence at the following nucleotide positions: 419 to 440.

SGK124 (SEQ ID NO:9, ENCODING SEQ ID NO:66) is 1077 nucleotides long. The open reading frame starts at position 1 and ends at position 1074, giving a ORF length of 1074 nucleotides. The predicted protein is 358 amino acids long. This sequence is full length (start methionine to stop codon).It is classified as (superfamily/group/family): protein kinase, CAMK, AMPK. This gene maps to chromosomal position 20p12.2-p13. This chromosomal position has been associated with the following human diseases: cancer of the gastroesophageal junction (20p12; 3/28), (Knuutila, et al.); familial noncompaction of left ventricle (OMIM, 604169 NONCOMPACTION OF LEFT VENTRICULAR MYOCARDIUM, FAMILIAL ISOLATED, AUTOSOMAL DOMINANT, 20p13). This gene contains candidate single nucleotide polymorphisms at the following positions: 188=S; 333=Y. ESTs for this gene in the public domain (dbEST) are: BF026145, BE298893. This gene has repetitive sequence at the following nucleotide positions: none.

SGK254, CAMKKa (SEQ ID NO:10, ENCODING SEQ ID NO:67) is 1542 nucleotides long. The open reading frame starts at position 1 and ends at position 1539, giving a ORF length of 1539 nucleotides. The predicted protein is 513 amino acids long. This sequence is full length (start methionine to stop codon).It is classified as (superfamily/group/family): protein kinase, CAMK, CAMK. This gene maps to chromosomal position 17p13.3. This chromosomal position has been associated with the following human diseases: Lost in cervical cancer (loss of heterogeneity, Lazo, The molecular genetics of cervical carcinoma. Br J Cancer. 1999 August;80(12):2008-18. Review). This gene contains candidate single nucleotide polymorphisms at the following positions: 555=R (ss84265); 1148=R. ESTs for this gene in the public domain (dbEST) are: BE783149. This gene has repetitive sequence at the following nucleotide positions: none.

SGK297, CaMKIb2 (SEQ ID NO:I 1, ENCODING SEQ ID NO:68) is 1032 nucleotides long. The open reading frame starts at position 1 and ends at position 1029, giving a ORF length of 1029 nucleotides. The predicted protein is 343 amino acids long. This sequence is full length (start methionine to stop codon).It is classified as (superfamily/group/family): protein kinase, CAMK, CAMK. This gene maps to chromosomal position Xq28. Translocations involving this chromosomal position has been associated with the following human diseases: human T cell prolymphocytic leukemia (Laine, et al.Mol Cell. 2000 August;6(2):395-407); mental retardation (e.g., Russo, et al., Amr J Med Genet. 2000 October 23;94(5):376-82). Also in Mantle cell lymphoma (Xq26-q28, 5/50).(Knuutila, et al.). This gene contains candidate single nucleotide polymorphisms at the following positions: 77=K. ESTs for this gene in the public domain (dbEST) are: AI696123, A1141657. This gene has repetitive sequence at the following nucleotide positions: none.

SGK411, CaMKII delta2 (SEQ ID NO:12, ENCODING SEQ ID NO:69) is 1500 nucleotides long. The open reading frame starts at position 1 and ends at position 1497, giving a ORF length of 1497 nucleotides. The predicted protein is 499 amino acids long. This sequence is full length (start methionine to stop codon).It is classified as (superfamily/group/family): protein kinase, CAMK, CAMK. This gene maps to chromosomal position 4q25. This chromosomal position has been associated with the following human diseases: developmental glaucoma (Rieger syndrome, iris hypoplasia, and iridogoniodysgenesis;Craig, et al. Curr Opin Ophthalmol. 1999 Apr; 10(2):126-34) This gene contains candidate single nucleotide polymorphisms at the following positions: 15=M; 1387=S (ss1531091). ESTs for this gene in the public domain (dbEST) are: AW502248, AW504981, AA316038. This gene has repetitive sequence at the following nucleotide positions: none.

SGK027 (SEQ ID NO: 13, ENCODING SEQ ID NO:70) is 1311 nucleotides long. The open reading frame starts at position 1 and ends at position 1308, giving a ORF length of 1308 nucleotides. The predicted protein is 436 amino acids long. This sequence is full length (start methionine to stop codon).It is classified as (superfamily/group/family): protein kinase, CAMK, EMK. This gene maps to chromosomal position 5q11-q11.1. This chromosomal position has been associated with the following human diseases: Malignant fibrous histiocytoma of bone (1/26) (Knuutila, et al.). Also with B-cell non-Hodgkin's lymphoma. (Wlodarska, et al.Cytogenet Cell Genet. 1994;65(3):179-83). This gene contains candidate single nucleotide polymorphisms at the following positions: 45=Y. ESTs for this gene in the public domain (dbEST) are: BE551926, AI609751, AW138653. This gene has repetitive sequence at the following nucleotide positions: none.

SGK046b (SEQ ID NO:14, ENCODING SEQ ID NO:71) is 225 nucleotides long. The open reading frame starts at position 1 and ends at position 225, giving a ORF length of 225 nucleotides. The predicted protein is 75 amino acids long. This sequence is a partial kinase catalytic domain. It is classified as (superfamily/group/family): protein kinase, CAMK, EMK. This gene maps to chromosomal position 3p24.1. This chromosomal position has been associated with the following human diseases: Malignant fibrous histiocytoma of soft tissue (3p24-p26, 2/30) (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK046c (SEQ ID NO:15, ENCODING SEQ ID NO:72) is 117 nucleotides long. The open reading frame starts at position 1 and ends at position 117, giving a ORF length of 117 nucleotides. The predicted protein is 39 amino acids long. This sequence is a partial kinase catalytic domain. It is classified as (superfamily/group/family): protein kinase, CAMK, EMK. This gene maps to chromosomal position 3p24.1. This chromosomal position has been associated with the following human diseases: Malignant fibrous histiocytoma of soft tissue (3p24-p26, 2/30) (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK089 (SEQ ID NO:16, ENCODING SEQ ID NO:73) is 252 nucleotides long. The open reading frame starts at position 1 and ends at position 252, giving a ORF length of 252 nucleotides. The predicted protein is 84 amino acids long. This sequence is a partial kinase catalytic domain. It is classified as (superfamily/group/family): protein kinase, CAMK, EMK. This gene maps to chromosomal position 3p25.3. This chromosomal position has not been associated with human diseases This gene contains candidate single nucleotide polymorphisms at the following positions: none detected. ESTs for this gene in the public domain (dbEST) are: none. This gene has repetitive sequence at the following nucleotide positions: none.

SGK133 (SEQ ID NO:17, ENCODING SEQ ID NO:74) is 2385 nucleotides long. The open reading frame starts at position 1 and ends at position 2382, giving a ORF length of 2382 nucleotides. The predicted protein is 794 amino acids long. This sequence is full length (start methionine to stop codon).It is classified as (superfamily/group/family): protein kinase, CAMK, EMK. This gene maps to chromosomal position 7p11.2-p21. This chromosomal position has been associated with the following human diseases: Malignant fibrous histiocytoma of bone (1/26), (Knuutila, et al.). This gene contains candidate single nucleotide polymorphisms at the following positions: 2003=S;1673=S. ESTs for this gene in the public domain (dbEST) are: BE222941. This gene has repetitive sequence at the following nucleotide positions: none.

SGK004, MSK (SEQ ID NO:18, ENCODING SEQ ID NO:75) is 2361 nucleotides long. The open reading frame starts at position 1 and ends at position 2358, giving a ORF length of 2358 nucleotides. The predicted protein is 786 amino acids long. This sequence is full length (start methionine to stop codon). It is classified as (superfamily/group/family): protein kinase, CAMK, EMK-1. This gene maps to chromosomal position 21q22.3. A gene which causes severe ocular alterations and occipital encephalocele (Knobloch syndrome) is mapped to 21 q223 (Sertie, et al, Hum Mol Genet 1996 Jun;5(6):843-7). This gene contains candidate single nucleotide polymorphisms at the following positions: 1853=Y (ss571239). ESTs for this gene in the public domain (dbEST) are: AW503500. This gene has repetitive sequence at the following nucleotide positions: none.

SGK006 (SEQ ID NO:19, ENCODING SEQ ID NO:76) is 789 nucleotides long. The open reading frame starts at position 1 and ends at position 789, giving a ORF length of 789 nucleotides. The predicted protein is 263 amino acids long. This sequence contains the entire catalytic region of a novel kinase. It is classified as (superfamily/group/family): protein kinase, CAMK, EMK-1. This gene maps to chromosomal position 16q 16.1. This chromosomal position has not been associated with human diseases This gene contains candidate single nucleotide polymorphisms at the following positions: 4=S (ss1609852). ESTs for this gene in the public domain (dbEST) are: none. This gene has repetitive sequence at the following nucleotide positions: none.

SGK180, SNRK (SEQ ID NO:20, ENCODING SEQ ID NO:77) is 2298 nucleotides long. The open reading frame starts at position 1 and ends at position 2295, giving a ORF length of 2295 nucleotides. The predicted protein is 765 amino acids long. This sequence is full length (start methionine to stop codon).It is classified as (superfamily/group/family): protein kinase, CAMK, EMK-1. This gene maps to chromosomal position 3p21.31. This chromosomal position has been associated with the following human diseases: cancer of the bladder (1/14), (Knuutila, et al.). This gene contains candidate single nucleotide polymorphisms at the following positions: 1817=S. ESTs for this gene in the public domain (dbEST) are: AA447812, A1379954, AA199639. This gene has repetitive sequence at the following nucleotide positions: 1332 to 1353.

SGK386, MLCKs (SEQ ID NO:21, ENCODING SEQ ID NO:78) is 1836 nucleotides long. The open reading frame starts at position 1 and ends at position 1836, giving a ORF length of 1836 nucleotides. The predicted protein is 612 amino acids long. This sequence is full length (start methionine to stop codon).It is classified as (superfamily/group/family): protein kinase, CAMK, MLCK. This gene maps to chromosomal position 20q11.1. This chromosomal position has been associated with the following human diseases: papillary renal cell carcinoma. (Lab Invest. 1999 March;79(3):311-6). This gene contains candidate single nucleotide polymorphisms at the following positions: 835=M. ESTs for this gene in the public domain (dbEST) are: AA197072, R02824 . This gene has repetitive sequence at the following nucleotide positions: none.

SGK003 (SEQ ID NO:22, ENCODING SEQ ID NO:79) is 1014 nucleotides long. The open reading frame starts at position 1 and ends at position 1014, giving a ORF length of 1014 nucleotides. The predicted protein is 337 amino acids long. This sequence is full length (start methionine to stop codon).It is classified as (superfamily/group/family): protein kinase, CKI, CKI. This gene maps to chromosomal position 13q14.11. This chromosomal position has been associated with the following human diseases: fallopian tube cancer (13ql4-qter; 1/12) (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK066 (SEQ ID NO:23, ENCODING SEQ ID NO:80) is 1200 nucleotides long. The open reading frame starts at position 1 and ends at position 1200, giving a ORF length of 1200 nucleotides. The predicted protein is 400 amino acids long. This sequence contains the entire catalytic region of a novel kinase. It is classified as (superfamily/group/family): protein kinase, CKI, CKI. This gene maps to chromosomal position 15q15. This chromosomal position has been associated with the following human diseases: cancer of the testis (2/11), (Knuutila, et al.). This gene contains candidate single nucleotide polymorphisms at the following positions: none detected. ESTs for this gene in the public domain (dbEST) are: AA234451. This gene has repetitive sequence at the following nucleotide positions: 486 to 504.

SGK041, NKIAMRE (SEQ ID NO:24, ENCODING SEQ ID NO:81) is 1773 nucleotides long. The open reading frame starts at position 1 and ends at position 1773, giving a ORF length of 1773 nucleotides. The predicted protein is 591 amino acids long. This sequence is full length (start methionine to stop codon). It is classified as (superfamily/group/family): protein kinase, CMGC, CDK. This gene maps to chromosomal position 5q31.1. This chromosomal position has been associated with the following human diseases: cancer of the digestive tract (5q31-qter), (Knuutila, et al.). This gene contains candidate single nucleotide polymorphisms at the following positions: 1033=R;1284=R; 1181=Y. ESTs for this gene in the public domain (dbEST) are: A1684625, A1694352, A1912547. This gene has repetitive sequence at the following nucleotide positions: none.

SGK112 (SEQ ID NO:25, ENCODING SEQ ID NO:82) is 1083 nucleotides long. The open reading frame starts at position 1 and ends at position 1080, giving a ORF length of 1080 nucleotides. The predicted protein is 360 amino acids long. This sequence is full length (start methionine to stop codon). It is classified as (superfamily/group/family): protein kinase, CMGC, CDK. This gene maps to chromosomal position CHR2. This gene contains candidate single nucleotide polymorphisms at the following positions: none detected. ESTs for this gene in the public domain (dbEST) are: AA626859. This gene has repetitive sequence at the following nucleotide positions: none.

SGK038, ERK7 (SEQ ID NO:26, ENCODING SEQ ID NO:83) is 1677 nucleotides long. The open reading frame starts at position 1 and ends at position 1674, giving a ORF length of 1674 nucleotides. The predicted protein is 558 amino acids long. This sequence is full length (start methionine to stop codon). It is classified as (superfamily/group/family): protein kinase, CMGC, MAPK. This gene maps to chromosomal position na. This gene contains candidate single nucleotide polymorphisms at the following positions: none detected. ESTs for this gene in the public domain (dbEST) are: BE464560, AI049667. This gene has repetitive sequence at the following nucleotide positions: 508 to 528.

SGK158 (SEQ ID NO:27, ENCODING SEQ ID NO:84) is 2256 nucleotides long. The open reading frame starts at position 1 and ends at position 2253, giving a ORF length of 2253 nucleotides. The predicted protein is 751 amino acids long. This sequence is full length (start methionine to stop codon). It is classified as (superfamily/group/family): protein kinase, Microbial PK, ABC1. This gene maps to chromosomal position 1q42.11-42.2. This chromosomal position has not been associated with human diseases This gene contains candidate single nucleotide polymorphisms at the following positions: 1752=Y (ss 1529336). ESTs for this gene in the public domain (dbEST) are: BE797060, AW006971, A181941 1. This gene has repetitive sequence at the following nucleotide positions: 1246 to 1265.

SGK429 (SEQ ID NO:28, ENCODING SEQ ID NO:85) is 1881 nucleotides long. The open reading frame starts at position 1 and ends at position 1878, giving a ORF length of 1878 nucleotides. The predicted protein is 626 amino acids long. This sequence is full length (start methionine to stop codon). It is classified as (superfamily/group/family): protein kinase, Microbial PK, ABC1. This gene maps to chromosomal position 7q34-35. This chromosomal position has been associated with the following human diseases: deafness ( Mustapha, et al. Eur J Hum Genet. 1998 May-June;6(3):245-50). This gene contains candidate single nucleotide polymorphisms at the following positions: 196=R; 919=Y (ss1549835); 1865=Y (ss1517749). ESTs for this gene in the public domain (dbEST) are: BE877541, BE745459, BE259124. This gene has repetitive sequence at the following nucleotide positions: 1630 to 1651.

SGK152, SUDD (SEQ ID NO:29, ENCODING SEQ ID NO:86) is 1560 nucleotides long. The open reading frame starts at position 1 and ends at position 1557, giving a ORF length of 1557 nucleotides. The predicted protein is 519 amino acids long. This sequence is full length (start methionine to stop codon). It is classified as (superfamily/group/family): protein kinase, Microbial PK, R101. This gene maps to chromosomal position 18p11.1. This chromosomal position has not been associated with human diseases This gene contains candidate single nucleotide polymorphisms at the following positions: 972=M. ESTs for this gene in the public domain (dbEST) are: BE621277, AA452706. This gene has repetitive sequence at the following nucleotide positions: none.

SGK077 (SEQ ID NO:30, ENCODING SEQ ID NO:87) is 2397 nucleotides long. The open reading frame starts at position 1 and ends at position 2394, giving a ORF length of 2394 nucleotides. The predicted protein is 798 amino acids long. This sequence is full length (start methionine to stop codon). It is classified as (superfamily/group/family): protein kinase, Other, C26C2_ce. This gene maps to chromosomal position 17p13.3. This chromosomal position has been associated with the following human diseases: Lost in cervical cancer (loss of heterogeneity, Lazo, The molecular genetics of cervical carcinoma. Br J Cancer. 1999 August;80(12):2008-18. Review). This gene contains candidate single nucleotide polymorphisms at the following positions: 390=Y (ssl658885); 61 =R (ss1629760); 985=Y (ss1629759). ESTs for this gene in the public domain (dbEST) are: AA504563, AW752337. This gene has repetitive sequence at the following nucleotide positions: none.

SGK093, Wnk3 (SEQ ID NO:31, ENCODING SEQ ID NO:88) is 4542 nucleotides long. The open reading frame starts at position 1 and ends at position 4539, giving a ORF length of 4539 nucleotides. The predicted protein is 1513 amino acids long. This sequence is full length (start methionine to stop codon). It is classified as (superfamily/group/family): protein kinase, Other, C26C2_ce. This gene maps to chromosomal position 17q21.1-2. This chromosomal position has been associated with the following human diseases: cancer of the ovary (17q21-qter, 3/47), (Knuutila, et al.). This gene contains candidate single nucleotide polymorphisms at the following positions: 3279=K; 4078=M. ESTs for this gene in the public domain (dbEST) are: BF000369, A1650786, AA872586. This gene has repetitive sequence at the following nucleotide positions: none.

SGK074 (SEQ ID NO:32, ENCODING SEQ ID NO:89) is 1065 nucleotides long. The open reading frame starts at position 1 and ends at position 1065, giving a ORF length of 1065 nucleotides. The predicted protein is 355 amino acids long. This sequence contains the entire catalytic region of a novel kinase. It is classified as (superfamily/group/family): protein kinase, Other, DYRK. This gene maps to chromosomal position 19p12-19q13. This chromosomal position has been associated with the following human diseases: Small cell lung cancer (2/22), (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK087 (SEQ ID NO:33, ENCODING SEQ ID NO:90) is 1887 nucleotides long. The open reading frame starts at position 1 and ends at position 1884, giving a ORF length of 1884 nucleotides. The predicted protein is 628 amino acids long. This sequence is full length (start methionine to stop codon). It is classified as (superfamily/group/family): protein kinase, Other, DYRK. This gene maps to chromosomal position 12p13.3. This chromosomal position has been associated with the following human diseases: hypertension (Disse-Nicodeme, et al. Am J Hum Genet. 2000 August;67(2):302-10). This gene contains candidate single nucleotide polymorphisms at the following positions: 269=R (ss88136). ESTs for this gene in the public domain (dbEST) are: BE243995. This gene has repetitive sequence at the following nucleotide positions: 1264 to 1283.

SGK295, KIS (SEQ ID NO:34, ENCODING SEQ ID NO:91) is 1260 nucleotides long. The open reading frame starts at position 1 and ends at position 1257, giving a ORF length of 1257 nucleotides. The predicted protein is 419 amino acids long. This sequence is full length (start methionine to stop codon). It is classified as (superfamily/group/family): protein kinase, Other, DYRK. This gene maps to chromosomal position 1q23.3. This chromosomal position has been associated with the following human diseases: Hematologic neoplasms (11q23-qter; 1/1), (Knuutila, et al.). This gene contains candidate single nucleotide polymorphisms at the following positions: 355=W. ESTs for this gene in the public domain (dbEST) are: BE895119, A1221234, BE145607. This gene has repetitive sequence at the following nucleotide positions: none.

SGK419 (SEQ ID NO:35, ENCODING SEQ ID NO:92) is 1986 nucleotides long. The open reading frame starts at position 1 and ends at position 1983, giving a ORF length of 1983 nucleotides. The predicted protein is 661 amino acids long. This sequence is full length (start methionine to stop codon). It is classified as (superfamily/group/family): protein kinase, Other, NAK. This gene maps to chromosomal position 4q24. This chromosomal position has been associated with the following human diseases: Malignant fibrous histiocytoma of soft tissue (14q24-q31; 1/58), (Knuutila, et al.). ESTs for this gene in the public domain (dbEST) are: A1735391, AW967339, AA703517. This gene has repetitive sequence at the following nucleotide positions: none.

SGK125, MYO3A (SEQ ID NO:36, ENCODING SEQ ID NO:93) is 4848 nucleotides long. The open reading frame starts at position 1 and ends at position 4845, giving a ORF length of 4845 nucleotides. The predicted protein is 1615 amino acids long. This sequence is full length (start methionine to stop codon). It is classified as (superfamily/group/family): protein kinase, Other, NinaC. This gene maps to chromosomal position 10p12.32. This chromosomal position has been associated with the following human diseases: Mantle cell lymphoma (10p12-p13; 2/45), (Knuutila, et al.). This gene contains candidate single nucleotide polymorphisms at the following positions: 3145=Y; 3204=Y. ESTs for this gene in the public domain (dbEST) are: AW196373, AA476697. This gene has repetitive sequence at the following nucleotide positions: none.

SGK445 (SEQ ID NO:37, ENCODING SEQ ID NO:94) is 204 nucleotides long. The open reading frame starts at position 1 and ends at position 204, giving a ORF length of 204 nucleotides. The predicted protein is 68 amino acids long. This sequence is a partial kinase catalytic domain. It is classified as (superfamily/group/family): protein kinase, Other, PLK. This gene maps to chromosomal position na. (Knuutila, et al.). This gene contains candidate single nucleotide polymorphisms at the following positions: none detected. ESTs for this gene in the public domain (dbEST) are: none. This gene has repetitive sequence at the following nucleotide positions: 183 to 200.

SGK127 (SEQ ID NO:38, ENCODING SEQ ID NO:95) is 2838 nucleotides long. The open reading frame starts at position I and ends at position 2835, giving a ORF length of 2835 nucleotides. The predicted protein is 945 amino acids long. This sequence is full length (start methionine to stop codon). It is classified as (superfamily/group/family): protein kinase, Other, RAF. This gene maps to chromosomal position 12q24.21. This chromosomal position has been associated with the following human diseases: cancer of the respiratory tract and of the female genital organs (12q24.2). (Knuutila, et al.). This gene contains candidate single nucleotide polymorphisms at the following positions: 501=S (ss2005786). ESTs for this gene in the public domain (dbEST) are: none. This gene has repetitive sequence at the following nucleotide positions: none.

SGK009, ANKRD3 (SEQ ID NO:39, ENCODING SEQ ID NO:96) is 2499 nucleotides long. The open reading frame starts at position 1 and ends at position 2496, giving a ORF length of 2496 nucleotides. The predicted protein is 832 amino acids long. This sequence is full length (start methionine to stop codon). It is classified as (superfamily/group/family): protein kinase, Other, RIP. This gene maps to chromosomal position 21q22.3. A gene which causes severe ocular alterations and occipital encephalocele (Knobloch syndrome) is mapped to 21q223 (Sertie, et al, Hum Mol Genet 1996 June;5(6):843-7) (Knuutila, et al.). This gene contains candidate single nucleotide polymorphisms at the following positions: none detected. ESTs for this gene in the public domain (dbEST) are: none. This gene has repetitive sequence at the following nucleotide positions: 427-452.

SGK421, STK22A, TSK1 (SEQ ID NO:40, ENCODING SEQ ID NO:97) is 1104 nucleotides long. The open reading frame starts at position 1 and ends at position 1101, giving a ORF length of 1101 nucleotides. The predicted protein is 367 amino acids long. This sequence is full length (start methionine to stop codon). It is classified as (superfamily/group/family): protein kinase, Other, STK22A. This gene maps to chromosomal position 5q31.1. This chromosomal position has been associated with the following human diseases: Chondrosarcoma (5q31-q32; 2/45), (Knuutila, et al.). This gene contains candidate single nucleotide polymorphisms at the following positions: 510=M (ss2055126); 279=R (ss2055125). ESTs for this gene in the public domain (dbEST) are: none. This gene has repetitive sequence at the following nucleotide positions: none.

SGK047 (SEQ ID NO:41, ENCODING SEQ ID NO:98) is 93 nucleotides long. The open reading frame starts at position 1 and ends at position 93, giving a ORF length of 93 nucleotides. The predicted protein is 31 amino acids long. This sequence is a partial kinase catalytic domain. It is classified as (superfamily/group/family): protein kinase, Other, STKR. This gene maps to chromosomal position 10p11.21. This chromosomal position has been associated with the following human diseases: Squamous cell carcinomas of the head and neck (10p11-p13; 1/30), (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK196 (SEQ ID NO:42, ENCODING SEQ ID NO:99) is 1053 nucleotides long. The open reading frame starts at position 1 and ends at position 1050, giving a ORF length of 1050 nucleotides. The predicted protein is 350 amino acids long. This sequence is full length (start methionine to stop codon). It is classified as (superfamily/group/family): protein kinase, Other, Unique. This gene maps to chromosomal position na. This gene contains candidate single nucleotide polymorphisms at the following positions: 99=R. ESTs for this gene in the public domain (dbEST) are: none. This gene has repetitive sequence at the following nucleotide positions: none.

SGK396 (SEQ ID NO:43, ENCODING SEQ ID NO:100) is 1419 nucleotides long. The open reading frame starts at position 1 and ends at position 1416, giving a ORF length of 1416 nucleotides. The predicted protein is 472 amino acids long. This sequence is full length (start methionine to stop codon). It is classified as (superfamily/group/family): protein kinase, Other, Unique. This gene maps to chromosomal position na. This gene contains candidate single nucleotide polymorphisms at the following positions: none detected. ESTs for this gene in the public domain (dbEST) are: AL048857, AL048858, T09068. This gene has repetitive sequence at the following nucleotide positions: none.

SGK279, PKN (SEQ ID NO:44, ENCODING SEQ ID NO:101) is 1275 nucleotides long.

The open reading frame starts at position 1 and ends at position 1272, giving a ORF length of 1272 nucleotides. The predicted protein is 424 amino acids long. This sequence is full length (start methionine to stop codon). It is classified as (superfamily/group/family): protein kinase, Other, YWY3_ce. This gene maps to chromosomal position 16q22.3. This chromosomal position has been associated with the following human diseases: Diffuse large cell lymphoma of stomach (16q22-ter; 1/7), (Knuutila, et al.). This gene contains candidate single nucleotide polymorphisms at the following positions: 665=Y. ESTs for this gene in the public domain (dbEST) are: none. This gene has repetitive sequence at the following nucleotide positions: none.

SGK037 (SEQ ID NO:45, ENCODING SEQ ID NO:102) is 1947 nucleotides long. The open reading frame starts at position 1 and ends at position 1947, giving a ORF length of 1947 nucleotides. The predicted protein is 649 amino acids long. This sequence is full length (start methionine to stop codon). It is classified as (superfamily/group/family): protein kinase, STE, NEK. This gene maps to chromosomal position 13q14.12. This chromosomal position has been associated with the following human diseases: fallopian tube cancer (13q14-qter; 1/12) (Knuutila, et al.). This gene contains candidate single nucleotide polymorphisms at the following positions: none detected. ESTs for this gene in the public domain (dbEST) are: AA393108. This gene has repetitive sequence at the following nucleotide positions: 886 to 912.

SGK060 (SEQ ID NO:46, ENCODING SEQ ID NO:103) is 1938 nucleotides long. The open reading frame starts at position 1 and ends at position 1935, giving a ORF length of 1935 nucleotides. The predicted protein is 645 amino acids long. This sequence is full length (start methionine to stop codon). It is classified as (superfamily/group/family): protein kinase, STE, NEK. This gene maps to chromosomal position 3q22.1. This chromosomal position has been associated with the following human diseases: Mantle cell lymphoma (13q22-q32; 3/72), (Knuutila, et al.). This gene contains candidate single nucleotide polymorphisms at the following positions: 1463=W. ESTs for this gene in the public domain (dbEST) are: BE388672, AA412114. This gene has repetitive sequence at the following nucleotide positions: 465 to 488; 1468 to 1487.

SGK080 (SEQ ID NO:47, ENCODING SEQ ID NO:104) is 1338 nucleotides long. The open reading frame starts at position 1 and ends at position 1338, giving a ORF length of 1338 nucleotides. The predicted protein is 446 amino acids long. This sequence is full length (start methionine to stop codon). It is classified as (superfamily/group/family): protein kinase, STE, NEK. This gene maps to chromosomal position 22q11.2. This chromosomal position has been associated with the following human diseases: Non-small cell lung cancer (1/50), (Knuutila, et al.). This gene contains candidate single nucleotide polymorphisms at the following positions: 1159=R (ss1367671); 422=R (ss1855009). ESTs for this gene in the public domain (dbEST) are: none. This gene has repetitive sequence at the following nucleotide positions: none.

SGK002 (SEQ ID NO:48, ENCODING SEQ ID NO:105) is 1829 nucleotides long. The open reading frame starts at position 327 and ends at position 1523, giving a ORF length of 1197 nucleotides. The predicted protein is 399 amino acids long. This sequence is full length (start methionine to stop codon). It is classified as (superfamily/group/family): protein kinase, STE, STE11. This gene maps to chromosomal position 7q32.2. This chromosomal position has been associated with the following human diseases: Non-small cell lung cancer 7q32-q35; 1/50), (Knuutila, et al.). This gene contains candidate single nucleotide polymorphisms at the following positions: 1165=R; 983=M. ESTs for this gene in the public domain (dbEST) are: none. This gene has repetitive sequence at the following nucleotide positions: none.

SGK058 (SEQ ID NO:49, ENCODING SEQ ID NO:106) is 834 nucleotides long. The open reading frame starts at position 1 and ends at position 834, giving a ORF length of 834 nucleotides. The predicted protein is 278 amino acids long. This sequence contains the entire catalytic region of a novel kinase. It is classified as (superfamily/group/family): protein kinase, STE, STE11. This gene maps to chromosomal position 2q21.2. This chromosomal position has been associated with the following human diseases: bladder carcinoma (12q21-q24; 1/16), (Knuutila, et al.). This gene contains candidate single nucleotide polymorphisms at the following positions: none detected. ESTs for this gene in the public domain (dbEST) are: none. This gene has repetitive sequence at the following nucleotide positions: 733 to 751.

SGK103 (SEQ ID NO:50, ENCODING SEQ ID NO:107) is 84 nucleotides long. The open reading frame starts at position 1 and ends at position 84, giving a ORF length of 84 nucleotides. The predicted protein is 28 amino acids long. This sequence is a partial kinase catalytic domain. It is classified as (superfamily/group/family): protein kinase, STE, STE11. This gene maps to chromosomal position 5p14.3. This chromosomal position has been associated with the following human diseases: Malignant fibrous histiocytoma of soft tissue (6/88), (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK035 (SEQ ID NO:51, ENCODING SEQ ID NO:108) is 1044 nucleotides long. The open reading frame starts at position 1 and ends at position 1044, giving a ORF length of 1044 nucleotides. The predicted protein is 348 amino acids long. This sequence is a partial kinase catalytic domain. It is classified as (superfamily/group/family): protein kinase, STE, STE20. This gene maps to chromosomal position CHRI 5. This gene contains candidate single nucleotide polymorphisms at the following positions: 2273=Y. ESTs for this gene in the public domain (dbEST) are: none. This gene has repetitive sequence at the following nucleotide positions: 133 to 151; 695 to 713.

SGK075 (SEQ ID NO:52, ENCODING SEQ ID NO:109) is 3318 nucleotides long. The open reading frame starts at position 1 and ends at position 3318, giving a ORF length of 3318 nucleotides. The predicted protein is 1106 amino acids long. This sequence contains the entire catalytic region of a novel kinase. It is classified as (superfamily/group/family): protein kinase, STE, STE20. This gene maps to chromosomal position 2q31.1. This chromosomal position has been associated with the following human diseases: Squamous cell carcinomas of the head and neck (2q31-q33; 3/30), (Knuutila, et al.). This gene contains candidate single nucleotide polymorphisms at the following positions: 889=R. ESTs for this gene in the public domain (dbEST) are: none. This gene has repetitive sequence at the following nucleotide positions: none.

SGK188, EphA9 (SEQ ID NO:53, ENCODING SEQ ID NO:110) is 3112 nucleotides long. The open reading frame starts at position 74 and ends at position 3100, giving a ORF length of 3027 nucleotides. The predicted protein is 1009 amino acids long. This sequence is full length (start methionine to stop codon). It is classified as (superfamily/group/family): protein kinase, TK, RTK-11. This gene maps to chromosomal position 1p34.1-34.3. This chromosomal position has been associated with the following human diseases: cancer of the testis (1p34-pter; 1/11), (Knuutila, et al.). This gene contains candidate single nucleotide polymorphisms at the following positions: 2104=Y (ss1986120); 2319=R. ESTs for this gene in the public domain (dbEST) are: none. This gene has repetitive sequence at the following nucleotide positions: none.

SGK040 (SEQ ID NO:54, ENCODING SEQ ID NO: I I 1) is 2730 nucleotides long. The open reading frame starts at position 1 and ends at position 2727, giving a ORF length of 2727 nucleotides. The predicted protein is 909 amino acids long. This sequence is full length (start methionine to stop codon). It is classified as (superfamily/group/family): protein kinase, TK, Unique. This gene maps to chromosomal position 12q12. This chromosomal position has been associated with the following human diseases: Diffuse large cell lymphoma (2/66), (Knuutila, et al.). This gene contains candidate single nucleotide polymorphisms at the following positions: 1869=R, 1004=Y. ESTs for this gene in the public domain (dbEST) are: BE177830, AF114068, AA283608. This gene has repetitive sequence at the following nucleotide positions: 460 to 479.

SGK390 (SEQ ID NO:55, ENCODING SEQ ID NO:112) is 3495 nucleotides long. The open reading frame starts at position 1 and ends at position 3492, giving a ORF length of 3492 nucleotides. The predicted protein is 1164 amino acids long. This sequence is full length (start methionine to stop codon). It is classified as (superfamily/group/family): protein kinase-like, DAG kin, DAG kin. This gene maps to chromosomal position 13q14.2. This chromosomal position has been associated with the following human diseases: fallopian tube cancer (13q14-qter; 1/12) (Knuutila, et al.). This gene contains candidate single nucleotide polymorphisms at the following positions: 1314=R. ESTs for this gene in the public domain (dbEST) are: BE715967, A1333565, AW052032. This gene has repetitive sequence at the following nucleotide positions: none.

SGK007 (SEQ ID NO:56, ENCODING SEQ ID NO:113) is 2652 nucleotides long. The open reading frame starts at position 1 and ends at position 2652, giving a ORF length of 2652 nucleotides. The predicted protein is 884 amino acids long. This sequence contains the entire catalytic region of a novel kinase. It is classified as (superfamily/group/family): protein kinase-like, GCyc, GCyc. This gene maps to chromosomal position 10q26.11. This chromosomal position has not been associated with human diseases. (Knuutila, et al.). This gene contains candidate single nucleotide polymorphisms at the following positions: none detected. ESTs for this gene in the public domain (dbEST) are: none. This gene has repetitive sequence at the following nucleotide positions: none.

SGK050 (SEQ ID NO:57, ENCODING SEQ ID NO:114) is 144 nucleotides long. The open reading frame starts at position 1 and ends at position 144, giving a ORF length of 144 nucleotides. The predicted protein is 48 amino acids long. This sequence is a partial kinase catalytic domain. It is classified as (superfamily/group/family): protein kinase-like, GCyc, GCyc. This gene maps to chromosomal position 9p13.1-p13.2. This chromosomal position has been associated with the following human diseases: Non-small cell lung cancer (1/50) and testicular cancer (4/11) (Knuutila, et al.). This gene contains candidate single nucleotide polymorphisms at the following positions: none detected. ESTs for this gene in the public domain (dbEST) are: none. This gene has repetitive sequence at the following nucleotide positions: none.

### Example 2a

### Probe Generation

Genomic fragments were PCR cloned to be used as probes. Exon fragments were cloned from genomic DNA sources (HUVEC or HMEC) by PCR methodology. Annealing temperature for the oligos used was 68C in a 50 microliter reaction. 5 microliter aliquots were analyzed by agarose gel electrophoresis to verify the correct size fragment was obtained. Fragments of the correct size were excised from the agarose gel and gene-cleaned and subcloned into the pCR-TOPO4 vector (Stratagene). These ligated plasmids were transformed into E. coli bacteria (TOP10 strain/ Stratagene) and selected using ampicillin antibiotic resistance. Resulting colonies, four per construct, were grown in media containing ampicillin and the plasmid DNA purified. Restriction digest analysis was carried out to verify the correct DNA insert. Plasmids containing fragments of the correct size were sequence verified using the T7 and T3 primers. DNA fragments to be used as probes for the blots were generated by restriction digest and purification of these fragments. These fragments were radioactively labeled by incorporating alpha-32P dCTP using a Boehringer Mannheim Random Hexamer Labeling Kit. Incorporation of radioactive isotope resulted in probes generated with between 5×10⁷ to 2×10⁸ cpm per 100 nano-grams of DNA fragment. The PCR primers used to clone the fragments are listed below:

| | |
|---|---|
| (SEQ ID NO: 129) | |
| SGK003.5′ | GACGTTTATCTGGGCATCACCACC |
| (SEQ ID NO: 130) | |
| SGK003.3′ | TCGGACAGTGCCAATGAGGTGTTT |
| (SEQ ID NO: 131) | |
| SGK074.5′ | GTGATTGACTTCGGATCCGCCAGC |
| (SEQ ID NO: 132) | |
| SGK074.3′ | CTTTTCCCTTACCTTCGTCTCGGCCAG |
| (SEQ ID NO: 133) | |
| SGK077.5′ | CCGGGACCTGGGAGCCGGCTTTTC |
| (SEQ ID NO: 134) | |
| SGK077.3′ | GCTCGGGACGGCTGAGGCTGCAAC |
| (SEQ ID NO: 135) | |
| SGK124.5′ | AAGAAGCTGGTGCTGGAGAAGCTG |
| (SEQ ID NO: 136) | |
| SGK124.3′ | AACCACTTCTCTGTCTCCCTCCTC |
| (SEQ ID NO: 137) | |
| SGK187.5′ | TCCGACACCATAACTGAGTTACAG |
| (SEQ ID NO: 138) | |
| SGK187.3′ | CTGCTCCTGGGCCAATAAAGC |
| (SEQ ID NO: 139) | |
| SGK386.5′ | CCTGATGGGTGTCTCACCTCCTCT |
| (SEQ ID NO: 140) | |
| SGK386.3′ | GGAGATGATGGCAGGACAGCTGGG |
| (SEQ ID NO: 141) | |
| SGK396.5′ | TTGGAGGGCTCAGAAGAGGAC |
| (SEQ ID NO: 142) | |
| SGK396.3′ | AACAAGTCCCTCATCTCCAGGTGA |

### Example 2b

### Expression Analysis of Polypeptides of the Invention

The gene expression patterns for selected genes were studied using four techniques: 1) a tissue microarray developed at Sugen, containing over 450 tissues and probed with labeled genes; 2) a PCR screen of 48 human tissues (this technique does not yield quantitative expression levels between tissues, but does identify those tissues that express the gene at a level detectable by PCR, as well as those that do not express the gene at such a level), 3) a commercial array of tissue from Clontech, probed with labeled genes, and 4) for one gene (SGKO93), an analysis form Northern blotting.

1) Tissue Arrays

“cDNA libraries” derived from over 450 tissue or cell line sources were immobilized onto nylon membranes and probed with 32P-labeled cDNA fragments derived from the gene(s) of interest. To make the cDNA, total RNA or mRNA was used as template in a reverse transcription reaction to generate single-stranded cDNAs (ss cDNA) that were tagged with specific sequences at each end. An oligo dT primer containing a specific sequence (SEQ ID NO: 143) (CDS: AAGCAGTGGTAACAACGCAGAGTACT₃₀VN (V=A,G,C N=A,G,C,T)) anneals at the polyA track at the 3′ end of the mRNA and the reverse transcriptase (MMLV RnaseH⁻) transcribes the antisense strand until it reaches the end of the RNA strand when it adds additional C residues. If a primer (SEQ ID NO: 144) (SMII: AAGCAGTGGTAACAACGCAGAGTACGCGGG or (SEQ ID NO: 145) ML2G: AAGTGGCAACAGAGATAACGCGTACGCGGG) ending with 3 Gs is added, it anneals to the added Cs and the MMLV recognizes the rest of the primer sequence as template and continues transcription. As a result, the synthesized cDNAs contain specific sequence tags at both the 5′ and the 3′ end. When the 5′ and the 3′ ends are tagged with the same sequence (CDS and SMII) it is referred to as “symmetric”. When the 5′ end is tagged with a different sequence than the 3′ end (CDS and ML2G) is referred to as “asymmetric”. A double-stranded “cDNA library” is then generated by PCR amplification using the 3′PCR and ML2 primers (SEQ ID NO: 146) (3′ PCR: AAGCAGTGGTAACAACGCAGAGT and (SEQ ID NO: 147) ML2: AAGTGGCAACAGAGATAACGCGT) that anneal to the added sequence tags.

The amplified “cDNA libraries” were manually arrayed onto nylon membranes with a 384 pin replicator. The DNA was denatured by alkali treatment, neutralized and cross-linked by UV light. The arrays were pre-hybridized with Express Hyb (Clontech) and hybridized with ³²P labeled probes generated by random hexamer priming of cDNA fragments corresponding to the genes of interest. After washing, the blots were exposed to phosphorimaging cassettes and the intensity of the signal was quantified. The amount of the DNA on the arrays was also quantified by treating non-denatured or denatured arrays with Syber Green I or Syber Green II respectively (1:100,000 in 50 mM Tris, pH8.0) for 2 minutes. After washing with 50 mM Tris, pH8.0, the fluorescent emission was detected with a phosphorimager (Molecular Dynamics) and quantified. The amount of the arrayed DNA was used to normalize the hybridization signal and the corrected values are tabulated in Table 6.

Cell treatments. Several cell lines were treated with componds to evaulate their effects on gene expression. There were eight treatments: 1) contriol, 2) low sereum, 3) 200 uM mimosine, 4) 3 mM HU, 5) 2 uM AUR2 inhibitor,6) 10 uM cisplatin, 7) 400 ng/ml nocodozole-24 hours, and 8) 400 ng/ml nocodozole-48 hours. The treated cell lines are listed by cell line name followed by a number from 1 to 8.

### Example 2c

### Predicted Proteins

SGK187, CRIK (SEQ ID NO:1, ENCODING SEQ ID NO:58) encodes a protein that is 2053 amino acids long. It is classified as (superfamily/group/family): protein kinase, AGC, DMPK. The kinase domain in this protein matches the hidden Markov profile for a full length kinase domain of 261 amino acids from profile position 1 to profile position 261. The position of the kinase catalytic region within the encoded protein is from amino acid 98 to amino acid 361. The results of a Smith Waterman search of the public database of amino acid sequences (NRAA) with this protein sequence yielded the following results: Pscore=0; number of identical amino acids=1970; percent identity=96%; percent similarity=98%; the accession number of the most similar entry in NRAA is AAC72823.1; the name or description, and species, of the most similar protein in NRAA is: Rho/rac-interacting citron kinase [Mus musculus]. Domains other than the kinase catalytic domain identified within this protein, and their amino acid positions, are: CNH domain (1620-1917); PH 1472-1591; Protein kinase C terminal domain (362-391); Phorbol esters/diacylglycerol binding domain (Cl domain) (1391-1439)”.

SGK064, GRK7 (SEQ ID NO:2, ENCODING SEQ ID NO:59) encodes a protein that is 553 amino acids long. It is classified as (superfamily/group/family): protein kinase, AGC, GRK. The kinase domain in this protein matches the hidden Markov profile for a full length kinase domain of 261 amino acids from profile position 1 to profile position 261. The position of the kinase catalytic region within the encoded protein is from amino acid 191 to amino acid 454. The results of a Smith Waterman search of the public database of amino acid sequences (NRAA) with this protein sequence yielded the following results: Pscore=0; number of identical amino acids=466; percent identity=84%; percent similarity=91%; the accession number of the most similar entry in NRAA is AAC95001.1; the name or description, and species, of the most similar protein in NRAA is: G protein-coupled receptor kinase GRK7 [Spermophilus tridecemlineatus]. Domains other than the kinase catalytic domain identified within this protein are: RGS (55-176).

SGK409, KIAA0303 (SEQ ID NO:3, ENCODING SEQ ID NO:60) encodes a protein that is 2523 amino acids long. It is classified as (superfamily/group/family): protein kinase, AGC, MAST. The kinase domain in this protein matches the hidden Markov profile for a full length kinase domain of 261 amino acids from profile position 1 to profile position 261. The position of the kinase catalytic region within the encoded protein is from amino acid 470 to amino acid 743 (PFAM profile). The results of a Smith Waterman search of the public database of amino acid sequences (NRAA) with this protein sequence yielded the following results: Pscore=0; number of identical amino acids=2137; percent identity=100%; percent similarity=100%; the accession number of the most similar entry in NRAA is BAA20762.1; the name or description, and species, of the most similar protein in NRAA is: KIAA0303 [Homo sapiens]. Domains other than the kinase catalytic domain identified within this protein are: PDZ domain (1020-1148).

SGK021 (SEQ ID NO:4, ENCODING SEQ ID NO:61) encodes a protein that is 327 amino acids long. It is classified as (superfamily/group/family): protein kinase, AGC, Mo3C11.1_ce. The kinase domain in this protein matches the hidden Markov profile for a full length kinase domain of 261 amino acids from profile position 1 to profile position 261. The position of the kinase catalytic region within the encoded protein is from amino acid 23 to amino acid 263. The results of a Smith Waterman search of the public database of amino acid sequences (NRAA) with this protein sequence yielded the following results: Pscore=6.00E-138; number of identical amino acids =203; percent identity=77%; percent similarity=84%; the accession number of the most similar entry in NRAA is CAB76566.1; the name or description, and species, of the most similar protein in NRAA is: Serine/threonine protein kinase [Mus musculus].

SGK410 (SEQ ID NO:5, ENCODING SEQ ID NO:62) encodes a protein that is 631 amino acids long. It is classified as (superfamily/group/family): protein kinase, AGC, PKC. The kinase domain in this protein matches the hidden Markov profile for a full length kinase domain of 261 amino acids from profile position 1 to profile position 261. The position of the kinase catalytic region within the encoded protein is from amino acid 289 to amino acid 557. The results of a Smith Waterman search of the public database of amino acid sequences (NRAA) with this protein sequence yielded the following results: Pscore=0; number of identical amino acids=555; percent identity=95%; percent similarity=96%; the accession number of the most similar entry in NRAA is NP_{—}002731.1; the name or description, and species, of the most similar protein in NRAA is: Protein kinase C, iota [Homo sapiens]. Domains other than the kinase catalytic domain identified within this protein are: Protein kinase C terminal domain (558-624); Phorbol esters/diacylglycerol binding domain (C1 domain) (176-225); Octicosapeptide repeat (100-129).

SGK069 (SEQ ID NO:6, ENCODING SEQ ID NO:63) encodes a protein that is 572 amino acids long. It is classified as (superfamily/group/family): protein kinase, AGC, Unique. The kinase domain in this protein matches the hidden Markov profile for a full length kinase domain of 261 amino acids from profile position 1 to profile position 261. The position of the kinase catalytic region within the encoded protein is from amino acid 158 to amino acid 421. The results of a Smith Waterman search of the public database of amino acid sequences (NRAA) with this protein sequence yielded the following results: Pscore=2.10E-64; number of identical amino acids=116; percent identity=42%; percent similarity=58%; the accession number of the most similar entry in NRAA is BAA36362. 1; the name or description, and species, of the most similar protein in NRAA is: Serine/threonine protein kinase [Rattus norvegicus].

SGK110 (SEQ ID NO:7, ENCODING SEQ ID NO:64) encodes a protein that is 373 amino acids long. It is classified as (superfamily/group/family): protein kinase, AGC, Unique. The kinase domain in this protein matches the hidden Markov profile for a full length kinase domain of 261 amino acids from profile position 1 to profile position 261. The position of the kinase catalytic region within the encoded protein is from amino acid 68 to amino acid 329. The results of a Smith Waterman search of the public database of amino acid sequences (NRAA) with this protein sequence yielded the following results: Pscore=1.10E-65; number of identical amino acids=112; percent identity=40%; percent similarity=58%; the accession number of the most similar entry in NRAA is S71887; the name or description, and species, of the most similar protein in NRAA is: pk9.7 gastrula-specific PK [Xenopus laevis].

SGK053, CKLiK (SEQ ID NO:8, ENCODING SEQ ID NO:65) encodes a protein that is 357 amino acids long. It is classified as (superfamily/group/family): protein kinase, CAMK, AMPK. The kinase domain in this protein matches the hidden Markov profile for a full length kinase domain of 261 amino acids from profile position 1 to profile position 261. The position of the kinase catalytic region within the encoded protein is from amino acid 23 to amino acid 279. The results of a Smith Waterman search of the public database of amino acid sequences (NRAA) with this protein sequence yielded the following results: Pscore=1.60E-245; number of identical amino acids=357; percent identity=100%; percent similarity=100%; the accession number of the most similar entry in NRAA is NP_{—}065130.1; the name or description, and species, of the most similar protein in NRAA is: CamKI-like protein kinase [Homo sapiens].

SGK124 (SEQ ID NO:9, ENCODING SEQ ID NO:66) encodes a protein that is 358 amino acids long. It is classified as (superfamily/group/family): protein kinase, CAMK, AMPK. The kinase domain in this protein matches the hidden Markov profile for a full length kinase domain of 261 amino acids from profile position 1 to profile position 261. The position of the kinase catalytic region within the encoded protein is from amino acid 73 to amino acid 315. The results of a Smith Waterman search of the public database of amino acid sequences (NRAA) with this protein sequence yielded the following results: Pscore=5.10E-252; number of identical amino acids=358; percent identity=100%; percent similarity=100%; the accession number of the most similar entry in NRAA is CAB81634.1; the name or description, and species, of the most similar protein in NRAA is: Novel protein kinase [Homo sapiens].

SGK254, CAMKKa (SEQ ID NO:10, ENCODING SEQ ID NO:67) encodes a protein that is 513 amino acids long. It is classified as (superfamily/group/family): protein kinase, CAMK, CAMK. The kinase domain in this protein matches the hidden Markov profile for a full length kinase domain of 261 amino acids from profile position 1 to profile position 261. The position of the kinase catalytic region within the encoded protein is from amino acid 128 to amino acid 417. The results of a Smith Waterman search of the public database of amino acid sequences (NRAA) with this protein sequence yielded the following results: Pscore=2.0e-323; number of identical amino acids=470; percent identity=91%; percent similarity=94%; the accession number of the most similar entry in NRAA is A57156; the name or description, and species, of the most similar protein in NRAA is: Ca2+/calmodulin-dep. PK IV [Rattus norvegicus].

SGK297, CaMKIb2 (SEQ ID NO: 11, ENCODING SEQ ID NO:68) encodes a protein that is 343 amino acids long. It is classified as (superfamily/group/family): protein kinase, CAMK, CAMK. The kinase domain in this protein matches the hidden Markov profile for a full length kinase domain of 261 amino acids from profile position 1 to profile position 261. The position of the kinase catalytic region within the encoded protein is from amino acid 15 to amino acid 270. The results of a Smith Waterman search of the public database of amino acid sequences (NRAA) with this protein sequence yielded the following results: Pscore=1.40E-236; number of identical amino acids=341; percent identity=99%; percent similarity=100%; the accession number of the most similar entry in NRAA is AAF74509. 1; the name or description, and species, of the most similar protein in NRAA is: Ca2+/Calmodulin-dependent protein kinase I [Homo sapiens].

SGK411, CaMKII delta2 (SEQ ID NO:12, ENCODING SEQ ID NO:69) encodes a protein that is 499 amino acids long. It is classified as (superfamily/group/family): protein kinase, CAMK, CAMK. The kinase domain in this protein matches the hidden Markov profile for a full length kinase domain of 261 amino acids from profile position 1 to profile position 261. The position of the kinase catalytic region within the encoded protein is from amino acid 14 to amino acid 272. The results of a Smith Waterman search of the public database of amino acid sequences (NRAA) with this protein sequence yielded the following results: Pscore=0; number of identical amino acids=499; percent identity=100%; percent similarity=100%; the accession number of the most similar entry in NRAA is AAD20442. 1; the name or description, and species, of the most similar protein in NRAA is: Multifunctional CAMK II delta2 [Homo sapiens].

SGK027 (SEQ ID NO: 13, ENCODING SEQ ID NO:70) encodes a protein that is 436 amino acids long. It is classified as (superfamily/group/family): protein kinase, CAMK, EMK. The kinase domain in this protein matches the hidden Markov profile for a full length kinase domain of 261 amino acids from profile position 1 to profile position 261. The position of the kinase catalytic region within the encoded protein is from amino acid 74 to amino acid 325. The results of a Smith Waterman search of the public database of amino acid sequences (NRAA) with this protein sequence yielded the following results: Pscore=7.70E-101; number of identical amino acids=147; percent identity=56%; percent similarity=76%; the accession number of the most similar entry in NRAA is T22427; the name or description, and species, of the most similar protein in NRAA is: F49C5.4—[Caenorhabditis elegans].

SGK046b (SEQ ID NO:14, ENCODING SEQ ID NO:71) encodes a protein that is 75 amino acids long. It is classified as (superfamily/group/family): protein kinase, CAMK, EMK. The kinase domain in this protein matches the hidden Markov profile for a full length kinase domain of 261 amino acids from profile position 98 to profile position 175. The position of the kinase catalytic region within the encoded protein is from amino acid 1 to amino acid 75. The results of a Smith Waterman search of the public database of amino acid sequences (NRAA) with this protein sequence yielded the following results: Pscore=1.40E-14; number of identical amino acids=35; percent identity=47%; percent similarity=61%; the accession number of the most similar entry in NRAA is AAC33487. 1; the name or description, and species, of the most similar protein in NRAA is: R31237_{—}1, partial CDS [Homo sapiens].

SGK046c (SEQ ID NO:15, ENCODING SEQ ID NO:72) encodes a protein that is 39 amino acids long. It is classified as (superfamily/group/family): protein kinase, CAMK, EMK. The kinase domain in this protein matches the hidden Markov profile for a full length kinase domain of 261 amino acids from profile position 106 to profile position 144. The position of the kinase catalytic region within the encoded protein is from amino acid 1 to amino acid 39. The results of a Smith Waterman search of the public database of amino acid sequences (NRAA) with this protein sequence yielded the following results: Pscore=6.40E-07; number of identical amino acids=21; percent identity=54%; percent similarity=67%; the accession number of the most similar entry in NRAA is AAC33487. 1; the name or description, and species, of the most similar protein in NRAA is: R31237_{—}1, partial CDS [Homo sapiens].

SGK089 (SEQ ID NO: 16, ENCODING SEQ ID NO:73) encodes a protein that is 84 amino acids long. It is classified as (superfamily/group/family): protein kinase, CAMK, EMK. The kinase domain in this protein matches the hidden Markov profile for a full length kinase domain of 261 amino acids from profile position 130 to profile position 216. The position of the kinase catalytic region within the encoded protein is from amino acid 1 to amino acid 84. The results of a Smith Waterman search of the public database of amino acid sequences (NRAA) with this protein sequence yielded the following results: Pscore=2.40E-19; number of identical amino acids=39; percent identity=46%; percent similarity=71%; the accession number of the most similar entry in NRAA is AAA97437.1; the name or description, and species, of the most similar protein in NRAA is: Serine/threonine kinase [Caenorhabditis elegans].

SGK133 (SEQ ID NO:17, ENCODING SEQ ID NO:74) encodes a protein that is 794 amino acids long. It is classified as (superfamily/group/family): protein kinase, CAMK, EMK. The kinase domain in this protein matches the hidden Markov profile for a full length kinase domain of 261 amino acids from profile position 1 to profile position 261. The position of the kinase catalytic region within the encoded protein is from amino acid 50 to amino acid 301. The results of a Smith Waterman search of the public database of amino acid sequences (NRAA) with this protein sequence yielded the following results: Pscore=8.2e-3 18; number of identical amino acids=481; percent identity=75%; percent similarity=83%; the accession number of the most similar entry in NRAA is CAA07196.1; the name or description, and species, of the most similar protein in NRAA is: Putative serine/threonine protein kinase [Homo sapiens].

SGK004, MSK (SEQ ID NO: 18, ENCODING SEQ ID NO:75) encodes a protein that is 786 amino acids long. It is classified as (superfamily/group/family): protein kinase, CAMK, EMK-1. The kinase domain in this protein matches the hidden Markov profile for a full length kinase domain of 261 amino acids from profile position 1 to profile position 261. The position of the kinase catalytic region within the encoded protein is from amino acid 27 to amino acid 281. The results of a Smith Waterman search of the public database of amino acid sequences (NRAA) with this protein sequence yielded the following results: Pscore=0; number of identical amino acids=784; percent identity=100%; percent similarity=100%; the accession number of the most similar entry in NRAA is P57059; the name or description, and species, of the most similar protein in NRAA is: SNF1LK [Homo sapiens].

SGK006 (SEQ ID NO: 19, ENCODING SEQ ID NO:76) encodes a protein that is 262 amino acids long. It is classified as (superfamily/group/family): protein kinase, CAMK, EMK-1. The kinase domain in this protein matches the hidden Markov profile for a full length kinase domain of 261 amino acids from profile position 1 to profile position 261. The position of the kinase catalytic region within the encoded protein is from amino acid 25 to amino acid 261. The results of a Smith Waterman search of the public database of amino acid sequences (NRAA) with this protein sequence yielded the following results: Pscore=1.50E-62; number of identical amino acids=121; percent identity=45%; percent similarity=66%; the accession number of the most similar entry in NRAA is NP_{—}056570. 1; the name or description, and species, of the most similar protein in NRAA is: Hormonally upregulated Neu-associated kinase [Mus musculus].

SGK180, SNRK (SEQ ID NO:20, ENCODING SEQ ID NO:77) encodes a protein that is 765 amino acids long. It is classified as (superfamily/group/family): protein kinase, CAMK, EMK-1. The kinase domain in this protein matches the hidden Markov profile for a full length kinase domain of 261 amino acids from profile position 1 to profile position 261. The position of the kinase catalytic region within the encoded protein is from amino acid 16 to amino acid 269. The results of a Smith Waterman search of the public database of amino acid sequences (NRAA) with this protein sequence yielded the following results: Pscore=0; number of identical amino acids=757; percent identity=99%; percent similarity=99%; the accession number of the most similar entry in NRAA is AAF86944.1; the name or description, and species, of the most similar protein in NkAA is: HSNFRK [Homo sapiens].

SGK386, MLCKs (SEQ ID NO:21, ENCODING SEQ ID NO:78) encodes a protein that is 612 amino acids long. It is classified as (superfamily/group/family): protein kinase, CAMK, MLCK. The kinase domain in this protein matches the hidden Markov profile for a full length kinase domain of 261 amino acids from profile position 1 to profile position 261. The position of the kinase catalytic region within the encoded protein is from amino acid 301 to amino acid 556. The results of a Smith Waterman search of the public database of amino acid sequences (NRAA) with this protein sequence yielded the following results: Pscore=0; number of identical amino acids=596; percent identity=97%; percent similarity=97%; the accession number of the most similar entry in NRAA is CAC 10006. 1; the name or description, and species, of the most similar protein in NRAA is: MYLK (myosin, light polypeptide kinase) [Homo sapiens].

SGK003 (SEQ ID NO:22, ENCODING SEQ ID NO:79) encodes a protein that is 337 amino acids long. It is classified as (superfamily/group/family): protein kinase, CKI, CKI. The kinase domain in this protein matches the hidden Markov profile for a full length kinase domain of 261 amino acids from profile position 1 to profile position 261. The position of the kinase catalytic region within the encoded protein is from amino acid 17 to amino acid 299. The results of a Smith Waterman search of the public database of amino acid sequences (NRAA) with this protein sequence yielded the following results: Pscore=1.40E-215; number of identical amino acids=304; percent identity=91%; percent similarity=95%; the accession number of the most similar entry in NRAA is NP_{—}001883.2; the name or description, and species, of the most similar protein in NRAA is: Casein kinase 1, alpha 1 [[Homo sapiens].

SGK066 (SEQ ID NO:23, ENCODING SEQ ID NO:80) encodes a protein that is 400 amino acids long. It is classified as (superfamily/group/family): protein kinase, CKI, CKI. The kinase domain in this protein matches the hidden Markov profile for a full length kinase domain of 261 amino acids from profile position 1 to profile position 261. The position of the kinase catalytic region within the encoded protein is from amino acid 21 to amino acid 281. The results of a Smith Waterman search of the public database of amino acid sequences (NRAA) with this protein sequence yielded the following results: Pscore=5.40E-106; number of identical amino acids=166; percent identity=53%; percent similarity=68%; the accession number of the most similar entry in NRAA is T24262; the name or description, and species, of the most similar protein in NRAA is: R90.1 [Caenorhabditis elegans].

SGK041, NKIAMRE (SEQ ID NO:24, ENCODING SEQ ID NO:81) encodes a protein that is 591 amino acids long. It is classified as (superfamily/group/family): protein kinase, CMGC, CDK. The kinase domain in this protein matches the hidden Markov profile for a full length kinase domain of 261 amino acids from profile position 1 to profile position 261. The position of the kinase catalytic region within the encoded protein is from amino acid 4 to amino acid 286. The results of a Smith Waterman search of the public database of amino acid sequences (NRAA) with this protein sequence yielded the following results: Pscore=1.0e-319; number of identical amino acids=454; percent identity=99%; percent similarity=100%; the accession number of the most similar entry in NRAA is NP_{—}057592.1; the name or description, and species, of the most similar protein in NRAA is: NKIAMRE [Homo sapiens].

SGK112 (SEQ ID NO:25, ENCODING SEQ. ID NO:82) encodes a protein that is 360 amino acids long. It is classified as (superfamily/group/family): protein kinase, CMGC, CDK. The kinase domain in this protein matches the hidden Markov profile for a full length kinase domain of 261 amino acids from profile position 1 to profile position 261. The position of the kinase catalytic region within the encoded protein is from amino acid 4 to amino acid 304. The results of a Smith Waterman search of the public database of amino acid sequences (NRAA) with this protein sequence yielded the following results: Pscore=8.70E-151; number of identical amino acids=224; percent identity=61%; percent similarity=74%; the accession number of the most similar entry in NRAA is NP_{—}004187.1; the name or description, and species, of the most similar protein in NRAA is: CDC2-related kinase [Homo sapiens].

SGK038, ERK7 (SEQ ID NO:26, ENCODING SEQ ID NO:83) encodes a protein that is 371 amino acids long. It is classified as (superfamily/group/family): protein kinase, CMGC, MAPK. The kinase domain in this protein matches the hidden Markov profile for a full length kinase domain of 261 amino acids from profile position 1 to profile position 261. The position of the kinase catalytic region within the encoded protein is from amino acid 13 to amino acid 323. The results of a Smith Waterman search of the public database of amino acid sequences (NRAA) with this protein sequence yielded the following results: Pscore=1.80E-105; number of identical amino acids=167; percent identity=59%; percent similarity=72%; the accession number of the most similar entry in NRAA is P51954; the name or description, and species, of the most similar protein in NRAA is: NEKI (NIMA-RELATED PROTEIN KINASE 1) [Mus musculus].

SGK158 (SEQ ID NO:27, ENCODING SEQ ID NO:84) encodes a protein that is 751 amino acids long. It is classified as (superfamily/group/family): protein kinase, Microbial PK, ABC1. The kinase domain in this protein matches the hidden Markov profile for a full length kinase domain of 261 amino acids from profile position 253 to profile position 261. The position of the kinase catalytic region within the encoded protein is from amino acid 522 to amino acid 532. The results of a Smith Waterman search of the public database of amino acid sequences (NRAA) with this protein sequence yielded the following results: Pscore=3.30E-257; number of identical amino acids=368; percent identity=100%; percent similarity=100%; the accession number of the most similar entry in NRAA is NP_{—}064632.1; the name or description, and species, of the most similar protein in NRAA is: Hypothetical protein [Homo sapiens].

SGK429 (SEQ ID NO:28, ENCODING SEQ ID NO:85) encodes a protein that is 626 amino acids long. It is classified as (superfamily/group/family): protein kinase, Microbial PK, ABC1. The kinase domain in this protein matches the hidden Markov profile for a full length kinase domain of 261 amino acids from profile position 1 to profile position 34. The position of the kinase catalytic region within the encoded protein is from amino acid 200 to amino acid 315. The results of a Smith Waterman search of the public database of amino acid sequences (NRAA) with this protein sequence yielded the following results: Pscore=6.00E-122; number of identical amino acids=191; percent identity=100%; percent similarity=100%; the accession number of the most similar entry in NRAA is AAD43192.1; the name or description, and species, of the most similar protein in NRAA is: Putative human protein [Homo sapiens].

SGK152, SUDD (SEQ ID NO:29, ENCODING SEQ ID NO:86) encodes a protein that is 519 amino acids long. It is classified as (superfamily/group/family): protein kinase, Microbial PK, R101. The kinase domain in this protein matches the hidden Markov profile for a full length kinase domain of 261 amino acids from profile position 251 to profile position 261. The position of the kinase catalytic region within the encoded protein is from amino acid 347 to amino acid 358. The results of a Smith Waterman search of the public database of amino acid sequences (NRAA) with this protein sequence yielded the following results: Pscore=0; number of identical amino acids=519; percent identity=100%; percent similarity=100%; the accession number of the most similar entry in NRAA is NP_{—}003822. 1; the name or description, and species, of the most similar protein in NRAA is: sudD (suppressor of bimD6, Aspergillus nidulans) homolog [Homo sapiens].

SGK077 (SEQ ID NO:30, ENCODING SEQ ID NO:87) encodes a protein that is 798 amino acids long. It is classified as (superfamily/group/family): protein kinase, Other, C26C2_ce. The kinase domain in this protein matches the hidden Markov profile for a full length kinase domain of 261 amino acids from profile position 1 to profile position 32. The position of the kinase catalytic region within the encoded protein is from amino acid 484 to amino acid 513. The results of a Smith Waterman search of the public database of amino acid sequences (NRAA) with this protein sequence yielded the following results: Pscore=8.50E-306; number of identical amino acids=492; percent identity=62%; percent similarity=73%; the accession number of the most similar entry in NRAA is BAB00640.1; the name or description, and species, of the most similar protein in NRAA is: Hapsin [Mus musculus].

SGK093, Wnk3 (SEQ ID NO:31, ENCODING SEQ ID NO:88) encodes a protein that is 1513 amino acids long. It is classified as (superfamily/group/family): protein kinase, Other, C26C2_ce. The kinase domain in this protein matches the hidden Markov profile for a full length kinase domain of 261 amino acids from profile position 1 to profile position 261. The position of the kinase catalytic region within the encoded protein is from amino acid 162 to amino acid 420. The results of a Smith Waterman search of the public database of amino acid sequences (NRAA) with this protein sequence yielded the following results: Pscore=2.10E-203; number of identical amino acids=321; percent identity=60%; percent similarity=73%; the accession number of the most similar entry in NRAA is AAF31483.1; the name or description, and species, of the most similar protein in NRAA is: Kinase deficient protein KDP [Homo sapiens]. Domains other than the kinase catalytic domain identified within this protein are: Cyclin (amino acids 1373 to 1410).

SGK074 (SEQ ID NO:32, ENCODING SEQ ID NO:89) encodes a protein that is 355 amino acids long. It is classified as (superfamily/group/family): protein kinase, Other, DYRK. The kinase domain in this protein matches the hidden Markov profile for a full length kinase domain of 261 amino acids from profile position 1 to profile position 261. The position of the kinase catalytic region within the encoded protein is from amino acid 6 to amino acid 342. The results of a Smith Waterman search of the public database of amino acid sequences (NRAA) with this protein sequence yielded the following results: Pscore=2.00E-107; number of identical amino acids=176; percent identity=50%; percent similarity=67%; the accession number of the most similar entry in NRAA is AAD41593.1; the name or description, and species, of the most similar protein in NRAA is: Myak-S [Mus musculus].

SGK087 (SEQ ID NO:33, ENCODING SEQ ID NO:90)encodes a protein that is 628 amino acids long. It is classified as (superfamily/group/family): protein kinase, Other, DYRK. The kinase domain in this protein matches the hidden Markov profile for a full length kinase domain of 261 amino acids from profile position 1 to profile position 261. The position of the kinase catalytic region within the encoded protein is from amino acid 213 to amino acid 509. The results of a Smith Waterman search of the public database of amino acid sequences (NRAA) with this protein sequence yielded the following results: Pscore=6.20E-1 83; number of identical amino acids=267; percent identity=100%; percent similarity=100%; the accession number of the most similar entry in NRAA is AAF91393.1; the name or description, and species, of the most similar protein in NRAA is: DYRK4 [Homo sapiens].

SGK295, KIS (SEQ ID NO:34, ENCODING SEQ ID NO:91) encodes a protein that is 419 amino acids long. It is classified as (superfamily/group/family): protein kinase, Other, DYRK., The kinase domain in this protein matches the hidden Markov profile for a full length kinase domain of 261 amino acids from profile position 1 to profile position 200. The position of the kinase catalytic region within the encoded protein is from amino acid 23 to amino acid 238. The results of a Smith Waterman search of the public database of amino acid sequences (NRAA) with this protein sequence yielded the following results: Pscore=1.10E-292; number of identical amino acids=415; percent identity=99%; percent similarity=100%; the accession number of the most similar entry in NRAA is NP_{—}058989.1; the name or description, and species, of the most similar protein in NRAA is: Kinase interacting with stathmin [Rattus norvegicus]. Domains other than the kinase catalytic domain identified within this protein are: RNA recognition motif (345-401).

SGK419 (SEQ ID NO:35, ENCODING SEQ ID NO:92) encodes a protein that is 661 amino acids long. It is classified as (superfamily/group/family): protein kinase, Other, NAK. The kinase domain in this protein matches the hidden Markov profile for a full length kinase domain of 261 amino acids from profile position 1 to profile position 261. The position of the kinase catalytic region within the encoded protein is from amino acid 51 to amino acid 341. The results of a Smith Waterman search of the public database of amino acid sequences (NRAA) with this protein sequence yielded the following results: Pscore=5.00E-252; number of identical amino acids=355; percent identity=100%; percent similarity=100%; the accession number of the most similar entry in NRAA is CAB70863. 1; the name or description, and species, of the most similar protein in NRAA is: Hypothetical protein [Homo sapiens].

SGK125, MYO3A (SEQ ID NO:36, ENCODING SEQ ID NO:93) encodes a protein that is 1615 amino acids long. It is classified as (superfamily/group/family): protein kinase, Other, NinaC. The kinase domain in this protein matches the hidden Markov profile for a full length kinase domain of 261 amino acids from profile position 1 to profile position 261. The position of the kinase catalytic region within the encoded protein is from amino acid 21 to amino acid 287. The results of a Smith Waterman search of the public database of amino acid sequences (NRAA) with this protein sequence yielded the following results: Pscore=0; number of identical amino acids=1615; percent identity=100%; percent similarity=100%; the accession number of the most similar entry in NRAA is NP_{—}059129. 1; the name or description, and species, of the most similar protein in NRAA is: Myosin IIIA [Homo sapiens]. Domains other than the kinase catalytic domain identified within this protein are: Myosin head (340-1040); IQ, (3 domains, 1055-1366).

SGK445 (SEQ ID NO:37, ENCODING SEQ ID NO:94) encodes a protein that is 68 amino acids long. It is classified as (superfamily/group/family): protein kinase, Other, PLK. The kinase domain in this protein matches the hidden Markov profile for a full length kinase domain of 261 amino acids from profile position 1 to profile position 71. The position of the kinase catalytic region within the encoded protein is from amino acid 1 to amino acid 68. The results of a Smith Waterman search of the public database of amino acid sequences (NRAA) with this protein sequence yielded the following results: Pscore=9.90E-25; number of identical amino acids=47; percent identity=65%; percent similarity=81%; the accession number of the most similar entry in NRAA is AAC37649.1; the name or description, and species, of the most similar protein in NRAA is: STK [Mus musculus].

SGK127 (SEQ ID NO:38, ENCODING SEQ ID NO:95) encodes a protein that is 945 amino acids long. It is classified as (superfamily/group/family): protein kinase, Other, RAF. The kinase domain in this protein matches the hidden Markov profile for a full length kinase domain of 261 amino acids from profile position 1 to profile position 261. The position of the kinase catalytic region within the encoded protein is from amino acid 661 to amino acid 922. The results of a Smith Waterman search of the public database of amino acid sequences (NRAA) with this protein sequence yielded the following results: Pscore=8.60E-254; number of identical amino acids=446; percent identity=47%; percent similarity=61%; the accession number of the most similar entry in NRAA is NP_{—}038599.1; the name or description, and species, of the most similar protein in NRAA is: Kinase suppressor of ras [Mus musculus]. Domains other than the kinase catalytic domain identified within this protein are: Phorbol esters/diacylglycerol binding domain (C1 domain) (408-451).

SGK009, ANKRD3 (SEQ ID NO:39, ENCODING SEQ ID NO:96) encodes a protein that is 832 amino acids long. It is classified as (superfamily/group/family): protein kinase, Other, RIP. The kinase domain in this protein matches the hidden Markov profile for a full length kinase domain of 261 amino acids from profile position 1 to profile position 261. The position of the kinase catalytic region within the encoded protein is from amino acid 22 to amino acid 276. The results of a Smith Waterman search of the public database of amino acid sequences (NRAA) with this protein sequence yielded the following results: Pscore=0; number of identical amino acids=784; percent identity=100%; percent similarity=100%; the accession number of the most similar entry in NRAA is NP_{—}065690.1; the name or description, and species, of the most similar protein in NRAA is: Ankyrin repeat domain 3 [Homo sapiens]. Domains other than the kinase catalytic domain identified within this protein are: “Ankyrin (10 domains: 437-469, 470-502, 503-535, 536-568, 569-602, 602-635, 636-668, 669-701, 702-730, 738-770)”.

SGK421, STK22A, TSK1 (SEQ ID NO:40, ENCODING SEQ ID NO:97) encodes a protein that is 367 amino acids long. It is classified as (superfamily/group/family): protein kinase, Other, STK22A. The kinase domain in this protein matches the hidden Markov profile for a full length kinase domain of 261 amino acids from profile position 1 to profile position 261. The position of the kinase catalytic region within the encoded protein is from amino acid 12 to amino acid 272. The results of a Smith Waterman search of the public database of amino acid sequences (NRAA) with this protein sequence yielded the following results: Pscore=6.40E-209; number of identical amino acids=307; percent identity=84%; percent similarity=90%; the accession number of the most similar entry in NRAA is NP_{—}033461.1; the name or description, and species, of the most similar protein in NRAA is: STK 22A (spermiogenesis associated) [Mus musculus].

SGK047 (SEQ ID NO:41, ENCODING SEQ ID NO:98) encodes a protein that is 31 amino acids long. It is classified as (superfamily/group/family): protein kinase, Other, STKR. The kinase domain in this protein matches the hidden Markov profile for a full length kinase domain of 261 amino acids from profile position 232 to profile position 261. The position of the kinase catalytic region within the encoded protein is from amino acid 1 to amino acid 31. The results of a Smith Waterman search of the public database of amino acid sequences (NRAA) with this protein sequence yielded the following results: Pscore=0.265466; number of identical amino acids=11; percent identity=46%; percent similarity=67%; the accession number of the most similar entry in NRAA is NP_{—}004320.1; the name or description, and species, of the most similar protein in NRAA is: Bone morphogenetic protein receptor, type IA [Homo sapiens].

SGK196 (SEQ ID NO:42, ENCODING SEQ ID NO:99) encodes a protein that is 350 amino acids long. It is classified as (superfamily/group/family): protein kinase, Other, Unique. The kinase domain in this protein matches the hidden Markov profile for a full length kinase domain of 261 amino acids from profile position 1 to profile position 88. The position of the kinase catalytic region within the encoded protein is from amino acid 81 to amino acid 160. The results of a Smith Waterman search of the public database of amino acid sequences (NRAA) with this protein sequence yielded the following results: Pscore=2.70E-248; number of identical amino acids=350; percent identity=100%; percent similarity=100%; the accession number of the most similar entry in NRAA is BAB 1 5623.1; the name or description, and species, of the most similar protein in NRAA is: Unnamed protein product [Homo sapiens].

SGK396 (SEQ ID NO:43, ENCODING SEQ ID NO: 100) encodes a protein that is 472 amino acids long. It is classified as (superfamily/group/family): protein kinase, Other, Unique. The kinase domain in this protein matches the hidden Markov profile for a full length kinase domain of 261 amino acids from profile position 1 to profile position 261. The position of the kinase catalytic region within the encoded protein is from amino acid 186 to amino acid 425. The results of a Smith Waterman search of the public database of amino acid sequences (NRAA) with this protein sequence yielded the following results: Pscore=3.00E-09; number of identical amino acids=34; percent identity=28%; percent similarity=50%; the accession number of the most similar entry in NRAA is BAB 11570. 1; the name or description, and species, of the most similar protein in NRAA is: Receptor-like protein kinase [Arabidopsis thaliana].

SGK279, PKN (SEQ ID NO:44, ENCODING SEQ ID NO:101) encodes a protein that is 424 amino acids long. It is classified as (superfamily/group/family): protein kinase, Other, YWY3_ce. The kinase domain in this protein matches the hidden Markov profile for a full length kinase domain of 261 amino acids from profile position 1 to profile position 261. The position of the kinase catalytic region within the encoded protein is from amino acid 53 to amino acid 313. The results of a Smith Waterman search of the public database of amino acid sequences (NRAA) with this protein sequence yielded the following results: Pscore=4.10E-276; number of identical amino acids=400; percent identity=94%; percent similarity=95%; the accession number of the most similar entry in NRAA is BAA36362. 1; the name or description, and species, of the most similar protein in NRAA is: Serine/threonine protein kinase [Rattus norvegicus].

SGK037 (SEQ ID NO:45, ENCODING SEQ ID NO:102) encodes a protein that is 649 amino acids long. It is classified as (superfamily/group/family): protein kinase, STE, NEK. The kinase domain in this protein matches the hidden Markov profile for a full length kinase domain of 261 amino acids from profile position 1 to profile position 261. The position of the kinase catalytic region within the encoded protein is from amino acid 4 to amino acid 259. The results of a Smith Waterman search of the public database of amino acid sequences (NRAA) with this protein sequence yielded the following results: Pscore=4.10E-56; number of identical amino acids=176; percent identity=65%; percent similarity=83%; the accession number of the most similar entry in NRAA is P51954; the name or description, and species, of the most similar protein in NRAA is: NEK1 [Mus musculus].

SGK060 (SEQ ID NO:46, ENCODING SEQ ID NO: 103) encodes a protein that is 645 amino acids long. It is classified as (superfamily/group/family): protein kinase, STE, NEK. The kinase domain in this protein matches the hidden Markov profile for a full length kinase domain of 261 amino acids from profile position 1 to profile position 261. The position of the kinase catalytic region within the encoded protein is from amino acid 29 to amino acid 287. The results of a Smith Waterman search of the public database of amino acid sequences (NRAA) with this protein sequence yielded the following results: Pscore=1.3e-322; number of identical amino acids=463; percent identity=100%; percent similarity=100%; the accession number of the most similar entry in NRAA is BAB 15672.1; the name or description, and species, of the most similar protein in NRAA is: Unnamed protein product [Homo sapiens].

SGK080 (SEQ ID NO:47, ENCODING SEQ ID NO:104) encodes a protein that is 446 amino acids long. It is classified as (superfamily/group/family): protein kinase, STE, NEK. The kinase domain in this protein matches the hidden Markov profile for a full length kinase domain of 261 amino acids from profile position 1 to profile position 261. The position of the kinase catalytic region within the encoded protein is from amino acid 8 to amino acid 269. The results of a Smith Waterman search of the public database of amino acid sequences (NRAA) with this protein sequence yielded the following results: Pscore=3.80E-266; number of identical amino acids=404; percent identity=90%; percent similarity=93%; the accession number of the most similar entry in NRAA is NP_{—}002488. 1; the name or description, and species, of the most similar protein in NRAA is: NIMA (never in mitosis gene a)-related kinase [Homo sapiens].

SGK002 (SEQ ID NO:48, ENCODING SEQ ID NO:105) encodes a protein that is 399 amino acids long. It is classified as (superfamily/group/family): protein kinase, STE, STE11. The kinase domain in this protein matches the hidden Markov profile for a full length kinase domain of 261 amino acids from profile position 1 to profile position 261. The position of the kinase catalytic region within the encoded protein is from amino acid 71 to amino acid 368. The results of a Smith Waterman search of the public database of amino acid sequences (NRAA) with this protein sequence yielded the following results: Pscore=3.10E-248; number of identical amino acids=369; percent identity=92%; percent similarity=94%; the accession number of the most similar entry in NRAA is P36507; the name or description, and species, of the most similar protein in NRAA is: MEK2 [Homo sapiens].

SGK058 (SEQ ID NO:49, ENCODING SEQ ID NO:106) encodes a protein that is 278 amino acids long. It is classified as (superfamily/group/family): protein kinase, STE, STE11. The kinase domain in this protein matches the hidden Markov profile for a full length kinase domain of 261 amino acids from profile position 1 to profile position 261. The position of the kinase catalytic region within the encoded protein is from amino acid 11 to amino acid 274. The results of a Smith Waterman search of the public database of amino acid sequences (NRAA) with this protein sequence yielded the following results: Pscore=3.00E-116; number of identical amino acids=167; percent identity=99%; percent similarity=99%; the accession number of the most similar entry in NRAA is BAB15538.1; the name or description, and species, of the most similar protein in NRAA is: Unnamed protein product [Homo sapiens].

SGK103 (SEQ ID NO:50, ENCODING SEQ ID NO:107) encodes a protein that is 28 amino acids long. It is classified as (superfamily/group/family): protein kinase, STE, STE11. The kinase domain in this protein matches the hidden Markov profile for a full length kinase domain of 261 amino acids from profile position 125 to profile position 148. The position of the kinase catalytic region within the encoded protein is from amino acid 1 to amino acid 24. The results of a Smith Waterman search of the public database of amino acid sequences (NRAA) with this protein sequence yielded the following results: Pscore=0.008942; number of identical amino acids=16; percent identity=57%; percent similarity=68%; the accession number of the most similar entry in NRAA is CAA39285.1; the name or description, and species, of the most similar protein in NRAA is: Fused [Drosophila melanogaster].

SGK035 (SEQ ID NO:51, ENCODING SEQ ID NO:108) encodes a protein that is 348 amino acids long. It is classified as (superfamily/group/family): protein kinase, STE, STE20. The kinase domain in this protein matches the hidden Markov profile for a full length kinase domain of 261 amino acids from profile position 1 to profile position 261. The position of the kinase catalytic region within the encoded protein is from amino acid 73 to amino acid 324. The results of a Smith Waterman search of the public database of amino acid sequences (NRAA) with this protein sequence yielded the following results: Pscore=7.60E-212; number of identical amino acids=318; percent identity=92%; percent similarity=95%; the accession number of the most similar entry in NRAA is Q13 177; the name or description, and species, of the most similar protein in NRAA is: PAK-2 [Homo sapiens].

SGK075 (SEQ ID NO:52, ENCODING SEQ ID NO:109) encodes a protein that is 1106 amino acids long. It is classified as (superfamily/group/family): protein kinase, STE, STE20. The kinase domain in this protein matches the hidden Markov profile for a full length kinase domain of 261 amino acids from profile position 1 to profile position 261. The position of the kinase catalytic region within the encoded protein is from amino acid 15 to amino acid 281. The results of a Smith Waterman search of the public database of amino acid sequences (NRAA) with this protein sequence yielded the following results: Pscore=4.00E-158; number of identical amino acids=227; percent identity=67%; percent similarity=80%; the accession number of the most similar entry in NRAA is NP_{—}059129.1; the name or description, and species, of the most similar protein in NRAA is: Myosin IIIA [Homo sapiens].

SGK188, EphA9 (SEQ ID NO:53, ENCODING SEQ ID NO:110) encodes a protein that is 1009 amino acids long. It is classified as (superfamily/group/family): protein kinase, TK, RTK-11. The kinase domain in this protein matches the hidden Markov profile for a full length kinase domain of 261 amino acids from profile position 1 to profile position 261. The position of the kinase catalytic region within the encoded protein is from amino acid 645 to amino acid 899. The results of a Smith Waterman search of the public database of amino acid sequences (NRAA) with this protein sequence yielded the following results: Pscore=0; number of identical amino acids=538; percent identity=54%; percent similarity=71%; the accession number of the most similar entry in NRAA is Q61772; the name or description, and species, of the most similar protein in NRAA is: Ephrin Type-A receptor 7 [Mus musculus]. Domains other than the kinase catalytic domain identified within this protein are: “Ephrin receptor ligand binding domain (35-211); Fibronectin type III domain (2 domains: 339-436 & 454-537); SAM domain (Sterile alpha motif)(931-996)”.

SGK040 (SEQ ID NO:54, ENCODING SEQ ID NO:111) encodes a protein that is 909 amino acids long. It is classified as (superfamily/group/family): protein kinase, TK, Unique. The kinase domain in this protein matches the hidden Markov profile for a full length kinase domain of 261 amino acids from profile position 1 to profile position 261. The position of the kinase catalytic region within the encoded protein is from amino acid 394 to amino acid 646. The results of a Smith Waterman search of the public database of amino acid sequences (NRAA) with this protein sequence yielded the following results: Pscore=4.50E-26; number of identical amino acids=76; percent identity=30%; percent similarity=52%; the accession number of the most similar entry in NRAA is P41243; the name or description, and species, of the most similar protein in NRAA is: BATK [Rattus norvegicus].

SGK390 (SEQ ID NO:55, ENCODING SEQ ID NO:112) encodes a protein that is 1164 amino acids long. It is classified as (superfamily/group/family): protein kinase-like, DAG kin, DAG kin. The kinase domain in this protein matches the hidden Markov profile for a full length kinase domain of 261 amino acids from profile position 1 to profile position 13. The position of the kinase catalytic region within the encoded protein is from amino acid 383 to amino acid 395. The results of a Smith Waterman search of the public database of amino acid sequences (NRAA) with this protein sequence yielded the following results: Pscore=0; number of identical amino acids=1063; percent identity=91%; percent similarity=94%; the accession number of the most similar entry in NRAA is Q64398; the name or description, and species, of the most similar protein in NRAA is: Diacylglycerol kinase eta [Mesocricetus auratus]. Domains other than the kinase catalytic domain identified within this protein are: “Diacylglycerol kinase catalytic domain (332-457);
DAG_PE-bind (176-225 & 248-298); PH domain (66-158)
Diacylglycerol kinase accessory domain (770-927);”.

SGK007 (SEQ ID NO:56, ENCODING SEQ ID NO:113) encodes a protein that is 884 amino acids long. It is classified as (superfamily/group/family): protein kinase-like, GCyc, GCyc. The kinase domain in this protein matches the hidden Markov profile for a full length kinase domain of 261 amino acids from profile position 166 to profile position 261. The position of the kinase catalytic region within the encoded protein is from amino acid 613 to amino acid 716. The results of a Smith Waterman search of the public database of amino acid sequences (NRAA) with this protein sequence yielded the following results: Pscore=5.20E-265; number of identical amino acids=499; percent identity=51%; percent similarity=62%; the accession number of the most similar entry in NRAA is T42260; the name or description, and species, of the most similar protein in NRAA is: Guanylate cyclase [Rattus norvegicus].

SGK050 (SEQ ID NO:57, ENCODING SEQ ID NO: 114) encodes a protein that is 48 amino acids long. It is classified as (superfamily/group/family): protein kinase-like, GCyc, GCyc. The kinase domain in this protein matches the hidden Markov profile for a full length kinase domain of 261 amino acids from profile position 51 to profile position 89. The position of the kinase catalytic region within the encoded protein is from amino acid 1 to amino acid 37. The results of a Smith Waterman search of the public database of amino acid sequences (NRAA) with this protein sequence yielded the following results: Pscore=1.30E-10; number of identical amino acids=48; percent identity=23%; percent similarity=48%; the accession number of the most similar entry in NRAA is NP_{—}003986.2; the name or description, and species, of the most similar protein in NRAA is: Natriuretic peptide receptor B precursor, isoform b [Homo sapiens].

Results

The results of the microarray expression analysis of the protein kinases presented in this application are shown, for example, in Tables 6 and 7. In Table 6, data presentation from left to right is as follows: “Tissue”: tissue type of the cDNA; “Tumor sym”, indicates that the tissue is derived from a tumor, “sym” refers to the fact that the 5′ and 3′ primers used to make the sample are the same; “Normal Sym”, indicates normal tissue was used to make the sample, with symmetric primers as described above; “Tumor lo”, indicates that primary tumor tissue was used to make Th cDNA; “Tumor cells”, indicates that these cDNA samples were made from cultured tumor cells; “Normal”, indicates that these samples are derived from normal tissue or cell lines; “Endos”, indicates that these samples are derived from endothelium-related tissue sources; “p53” refers to the status, mutant or wild-type, of the p53 gene in the source samples. Normalized expression values are presented for each gene referred to by its SEQ_ID# on the subsequent columns.

Genes represented in the tissue array are listed below: SGK187 (SEQ ID NO:1); SGK124 (SEQ ID NO:9); SGK386 (SEQ ID NO:21); SGK003 (SEQ ID NO:22); SGK077 (SEQ ID NO:30); SGK093 (SEQ ID NO:31); SGK074 (SEQ ID NO:32); and SGK396 (SEQ ID NO:43), as shown in Table 6.

SGK187 is expressed highest in the following tissues: heart, SA-OS cells, Prostate Tumor_{—}13, fetal liver, C33A, Prostate Tumor10. This expression pattern suggests this kinase may play a role in heart-related pathologies, and/or prostate cancer.

SGK074 is expressed highest in the following tissues: OVCAR-4-2, HCT-116-1, OVCAR-5-7, Prostate Tumor-09, Prostate Tumor-08, U20S-1, adrenal gland-h, Prostate Tumor-18, HT29-5, SW480-3. This expression pattern suggests expression of this gene may be associated with ovarian cancer, prostate cancer, colon cancer, and other forms of neoplasia.

Sgk093 is expressed highest in the following tissues: fetal kidney, kidney, prostate tumor, fetal liver, Caki-1, U20S-6, ACHN, normal prostate. This expression pattern suggests that this gene may be involved in diseases of the prostate, kidney and/or liver.

SGK396 is expressed highest in the following tissues: cerebellum, Prostate Tumor18, fetal brain, thymus-h, and lymph node. This expression pattern suggests this gene may be involved in hematopoietic disease, prostate cancer, or CNS abnormalities involving the cerebellum.

A statistical analysis of the gene expression patterns was carried out from the tissue array data. The tissue array data for the 8 kinases were standardized for statistical analysis across the different tissue types using range standardization. Standardization converts measurements to a common scale. We used range standardization, which subtracts the smallest value of each variable from each value and divides by its range. The new scale starts at 0 and ends at 1.0. The following statistical procedures were implemented on the standardized data: generation of descriptive statistics, graphical visualization, hierarchical and k-means cluster analysis, and comparison of groups using analysis of variance (ANOVA). When data was present for both normal and tumor tissues for particular tissue types, such groups were directly compared for fold differences. All statistical analyses were carried out separately for the symmetric and asymmetric tissue array laboratory methods (we know from experience with past data that gene expression is dependent upon the method used). All statistical analysis was carried out using SYSTAT 9.01 (Copyright©1999 by SPSS, Inc.).

Summary of Results:

1. SGKI87(NA#1)

(Symmetric Data)

Expressed highest in normal tissue of the human heart, fetal liver, and thymus.

Next highest expressions in the following tumor types: ovary, renal, lung, melanoma, colon, neuro.

Expressed in tumor but not normal tissue of the following tissue types: lung, prostate, renal.

1.4-fold higher expression in tumor vs. normal colon tissue.

A k-means cluster shows association of this kinase in renal tumor and ovary adenocarcinoma.

(Asymmetric Data)

Highest expression in tumors associated with the following tissue types: neuro, endo, ovary, breast, and colon.

Expressed in tumor but not normal sample of breast, colon, and renal tissue.

Over-expressed (tumor vs. normal) in the following tissues: neuro (3.4×) and pancreas (3.5×).

Under-expressed (tumor vs. normal) in the following tissues: lung (2.0×) and prostate (4.1×).

Singleton and tumor-associated small clusters (k-means) for this kinase were formed by neuro-metastases sample singly and in association with endo, neuro, and ovary tumors.

2. SGK124 (NA#9)

(Symmetric Data)

Highest expression in leukemia.

Next highest expressions in the following tissues: (1) normal: human keratinocy, liver, pituitary gland, fetal liver, lymph node, bone marrow, and fetal lung; and (2) tumor: melanoma, renal, neuro, lung, ovary, colon, and breast.

Expressed in tumor but not normal sample of breast tissue.

Over-expressed (tumor vs. normal) in the following tissues: colon (1.5×) and renal (5.5×).

Under-expressed (tumor vs. normal) in the following tissues: lung (2.8×) and neuro (3.2×); and expressed in normal but not tumor sample of testes.

A leukemia sample and a melanoma sample each form a singleton cluster (k-means) for this kinase.

(Asymmetric Data)

Highest expression in tumors associated with the following tissue types: neuro, endo, ovary, breast, colon, renal, and lung.

Expressed in tumor but not normal sample of breast and colon tissue.

Over-expressed (tumor vs. normal) in the following tissues: neuro (4.0×) and prostate (2.2×).

Under-expressed (tumor vs. normal) in the following tissues: endo (2.0×) and pancreas (5.3×).

Singleton clusters (k-means) for this kinase were formed by a bone tumor sample (h-osteogenic) and a neuro (metastases) sample. Other distinct tumor-associated clusters included one with cervical tumor and neuro-metastases, colon and lung tumor, and an association of adrenal gland with a metastases tumor sample.

3. SGK386 (NA#21)

(Symmetric Data)

Only expressed in a colon tumor sample. Zero expression for all other samples.

(Asymmetric Data)

Highest expression in tumors associated with the following tissue types: prostate metastases samples, endo, lung, ovary, breast, and colon.

Expressed in tumor but not normal sample of breast, colon, endo, and neuro.

Singleton and tumor-associated small clusters (k-means) for this kinase were formed by metastases samples, primarily prostate and neuro.

4. SGK003 (NA#22)

(Symmetric Data)

Highest expression in tumors of the colon and breast. Also high expression in normal tissue of the adrenal gland.

Over-expressed (tumor vs. normal) in the following tissues: colon (2.7×), breast (4.5×), and testes (4.6×).

Under-expressed (tumor vs. normal) in the following tissues: lung (2.1×), neuro (2.8×), prostate (10.1×), and renal (3.6×).

A colon adenocarcinoma sample forms a singleton cluster (k-means) for this kinase. A breast tumor sample forms another cluster in association with adrenal gland, both of which are high expressors for this kinase.

(Asymmetric Data)

Highest expression in tumors associated with the following tissue types: colon, lung, metastases-prostate, ovary, and bone.

Expressed in tumor but not normal sample of breast.

Over-expressed (tumor vs. normal) of the following tissues: colon (887×), endo (73×), neuro (75×), and renal (15×).

No under-expression (tumor vs. normal).

Singleton and tumor-associated small clusters (k-means) for this kinase were formed by prostate metastases samples with colon and lung tumors.

5. SGK077 (NA#30)

(Symmetric Data)

Highest expression in normal samples of human lung, cerebellum, pituitary gland, lymph node, fetal brain, thymus, pancreas, and placenta. Also relatively high expression in the following tumor types: neuro, breast, renal, colon, and melanoma.

Over-expressed (tumor vs. normal) in the following tissues: prostate (6.3×) and breast (3.7×).

Under-expressed (tumor vs. normal) in the following tissues: colon (1.7×), lung (22.9×), neuro (3.7×).

(Asymmetric Data)

Highest expression in metastases-prostate sample, normal human stomach, and tumor samples of endo, colon, neuro, lung, and ovary.

Over-expressed (tumor vs. normal) in neuro tissue (6.9×).

Under-expressed (tumor vs. normal) in the following tissue types: endo (2.0×), lung (3. 1×), pancreas (3.7×), and prostate (4.0×).

Most k-means clusters were formed by HUVEC normal samples, some of these in association with tumor samples.

6. SGKO93 (NA#31)

(Symmetric Data)

Highest expression in normal human adult and fetal kidney, fetal liver, and tumor samples of renal and lung.

Expressed in tumor but not normal sample of prostate tissue.

Over-expressed (tumor vs. normal) of breast tissue (1.4×).

Under-expressed (tumor vs. normal) in the following tissues: colon (2.0×), lung (1.9×), neuro (2.2×), and renal (15.9×).

Distinct k-means clusters were observed for the following singletons or associations: renal clear cell carcinoma (singleton), renal tumor (singleton), and fetal lung with lung carcinoma and lung adenocarcinoma (association).

(Asymmetric Data)

Highest expression in metastases samples and other tumors of bone, renal, ovary, and lung.

Expressed in tumor but not normal samples of colon and renal tissue.

Over-expressed (tumor vs. normal) in breast (2.3×) and lung (1.7×).

Under-expressed (tumor vs. normal) in the following tissue types: endo (4.0×), neuro (2.2×), and prostate (124×).

Singleton or very small k-means clusters were formed by metastases samples and one cluster was formed by lung adenocarcinoma in association with ovarian cancer.

7. SGK074 (NA#32)

(Symmetric Data)

Highest expression in normal adrenal gland and tumor samples of colon and breast.

Over-expressed (tumor vs. normal) in the following tissues: colon (1.4×), prostate (1.9×), and breast (2.3×).

Under-expressed (tumor vs. normal) in the following tissues: lung (2.1×), neuro (2.6×), renal (3.2×), and testes (2.1×).

Distinct k-means clusters were observed for the following singletons: colon adenocarcinoma and breast adenocarcinoma.

(Asymmetric Data)

Highest expression in a large variety of tumors including ovary, colon, metastases samples of prostate, bone, lung, neuro, cervical, breast, and endo.

Generally, very highly expressed in tumor as versus normal samples of a wide variety of tissues. Over-expressed (tumor vs. normal) in the following tissue types: breast (6.1×), colon (16.9×), endo (5.7×), neuro (9.1×), and pancreas (1.8×).

Under-expressed (tumor vs. normal) in the following tissue types: lung (1.5×), prostate (1.7×), and renal (2.7×).

Singleton or very small k-means clusters were formed by ovarian tumor and metastases samples in association with one another, and also in association with other tumor types.

8. SGK396 (NA#43)

(Symmetric Data)

Highest expression in the following normal tissues: cerebellum, fetal brain, thymus, lymph node, pituitary gland, fetal lung, and fetal kidney. Also very highly expressed in lung tumor samples and a host of other tumor samples: melanoma, renal, ovarian, leukemia, neuro, breast, and prostate.

Expressed in tumor but not normal samples of prostate and breast.

Over-expressed (tumor vs. normal) in testes (1.5×).

Under-expressed (tumor vs. normal) in the following tissues: colon (1.5×), lung (1.5×), neuro (8.1×), and renal (3.9×).

Two cancer dominated k-means clusters were observed, which represented a wide variety of tumor types.

(Asymmetric Data)

Highest expression in a large variety of tumors including prostate and neuro metastases samples, and tumors of bone, ovary, endo, breast, colon, and lung.

Over-expressed (tumor vs. normal) in the following tissue types: colon (6.3×) and neuro (4.0×)

Under-expressed (tumor vs. normal) in the following tissue types: endo (1.5×), lung (1.7×), pancreas (1.7×), prostate (4.2×), and renal (1.7×).

Singleton or very small k-means clusters were formed by different metastases samples from different tissue types, and in association with other tumor types.

PCR Screening:

Screening for Expression Sources by PCR from ds CDNA Templates

Preparation of dscDNA Templates

dscDNA templates were prepared by PCR amplification of symmetrically-tagged reverse transcriptase sscDNA products generated as described in detail under Materials and Methods for the Tissue Array Gene Expression protocol. The tissue sources amplified are listed, for example, in Table 7. The amplification conditions were as follows: per 200 microl of PCR reaction, added 100 microl of Premix TaKaRa ExTaq, 20.0 microl of pwo DNA polymerase (1/10 dilution made as follows: 1 microl pwo (5 units/microl), 1 microl lOx PCR buffer with 20 mM MgSO4, 8 microl water), 4.0 microl sscDNA template (reverse transcriptase product), 8.0 microl 10 pmoles/microl (10 microM) primer (AAGCAGTGGTAACAACGCAGAGT) (SEQ ID NO: 148) (1.0 microM final conc.) and 68.0 microl H₂O. The reaction was amplified according to the following regiment: hot start (95° C. for 1 min), 95° C. for 1 min, 24 cycles, 95° C. for 20 s, 65° C. for 30 s, 68° C. for 6 min, 68° C. for 10 min, 1 cycle and 4° C. forever. Following the PCR reaction, 5-10 microl of product were applied to an agarose gel together with 1 kb ladder size standards to assess the yield and uniformity of the product.

Source Identification

The following relates to Table 7. 5 microl of each of the 48 sources were used as a template with primers designed from the nucleotide sequence of the genes whose expression pattern were to be determined. The PCR conditions were the same as those used to make the dscDNA templates except for the following modifications: the PCR reaction was scaled down from 100 to 20 microl; pwo was omitted; 5 microl of template were used. The cycling conditions were the same as those used to make the dscDNA templates except that 35 instead of 24 cycles were used in the protocol. The primers used in these amplifications are listed below.

SGK069 (SEQ ID NO:6) was amplified from the 48 cDNA panel source using the following oligonucleotide primers: 5′ primer: (SEQ ID NO: 149) GGACATGATGACGCTGAGTGCTCAG; 3′ primer: (SEQ ID NO: 150) GTGCTCCCTGATGGCGATCACAGCG. Primer sequences were designed from partial or full-length versions of SEQ ID NO:6.

SGK110 (SEQ ID NO:7) was amplified from the 48 cDNA panel source using the following oligonucleotide primers: 5′ primer: (SEQ ID NO: 151) CTCCTGTCTTCTTGACCTCAGGGC; 3′ primer: (SEQ ID NO: 152) CAGGTCCAGAAGCCCCTGGAGC. Primer sequences were designed from partial or full-length versions of SEQ ID NO:7.

SGK053 (SEQ ID NO:8) was amplified from the 48 cDNA panel source using the following oligonucleotide primers: 5′ primer: (SEQ ID NO: 153) CCGAAAATCTCTTGTACTACAGTC; 3′ primer: (SEQ ID NO: 154) CATAGCCTGGAGTTCCACAGGCAG. Primer sequences were designed from partial or full-length versions of SEQ ID NO:8.

SGK254 (SEQ ID NO: 10) was amplified from the 48 cDNA panel source using the following oligonucleotide primers: 5′ primer: (SEQ ID NO: 155) GAGCCTACTAGAAACGGTGTGGACC; 3′ primer: (SEQ ID NO: 156) CTTCACCAAACCCTTCTTTCACCAG. Primer sequences were designed from partial or full-length versions of SEQ ID NO: 10.

SGK411 (SEQ ID NO: 12) was amplified from the 48 cDNA panel source using the following oligonucleotide primers: 5′ primer: (SEQ ID NO: 157) AAAGGCCCAGAGGGGACAGACGTGG; 3′ primer: (SEQ ID NO: 158) CCTCAATTGTTGTATTTGAACTCTC. Primer sequences were designed from partial or full-length versions of SEQ ID NO: 12.

SGK027 (SEQ ID NO: 13) was amplified from the 48 cDNA panel source using the following oligonucleotide primers: 5′ primer: (SEQ ID NO: 159) GAAAAGGTGGCCATTAAGATCCTGG; 3′ primer: (SEQ ID NO: 160) CATCCATTCATCATTCATGATGCAG. Primer sequences were designed from partial or full-length versions of SEQ ID NO: 13.

SGK046b (SEQ ID NO: 14) was amplified from the 48 cDNA panel source using the following oligonucleotide primers: 5′ primer: (SEQ ID NO: 161) GAAGAGGAGGAGGCCCAGACCATG; 3′ primer: (SEQ ID NO: 162) GGAAGAGTTCTGTGGCCACATAGG. Primer sequences were designed from partial or full-length versions of SEQ ID NO:14.

SGK089 (SEQ ID NO: 16) was amplified from the 48 cDNA panel source using the following oligonucleotide primers: 5′ primer: (SEQ ID NO: 163) CTTCTTTGACAAAGAACGTAACATC; 3′ primer: (SEQ ID NO: 164) CAGTATATCCTTACAGTTCTGCTC. Primer sequences were designed from partial or full-length versions of SEQ ID NO: 16.

SGK003 (SEQ ID NO:22) was amplified from the 48 cDNA panel source using the following oligonucleotide primers: 5′ primer: (SEQ ID NO: 165) CAACAGCGGCTCCAAAGCCGAACTC. 3′ primer: (SEQ IDNO: 166) TGCCTGTCTGGGTTTGGGCCTGCTG. Primer sequences were designed from partial or full-length versions of SEQ ID NO:22.

SGK066 (SEQ ID NO:23) was amplified from the 48 cDNA panel source using the following oligonucleotide primers: 5′ primer: (SEQ ID NO: 167) GTCCCGAGGCACATTCACCATTAG. 3′ primer: (SEQ ID NO: 168) GATGTCTCGATGCAAGAATCCCAC. Primer sequences were designed from partial or full-length versions of SEQ ID NO:23.

SGK041 (SEQ ID NO:24) was amplified from the 48 cDNA panel source using the following oligonucleotide primers: 5′ primer: (SEQ ID NO: 169) GAGATGTATGAAACCCTTGGAAAAG. 3′ primer: (SEQ ID NO: 170) CATACAGACACTATGAACACGATTC. Primer sequences were designed from partial or full-length versions of SEQ ID NO:24.

SGK038 (SEQ ID NO:26) was amplified from the 48 cDNA panel source using the following oligonucleotide primers: 5′ primer: (SEQ ID NO: 171) GCCTATGGCATTGTGTGGAAGGCAG. 3′ primer: (SEQ ID NO: 172) CAGCAGGGGCAGGTGGCCCAGTGCC. Primer sequences were designed from partial or full-length versions of SEQ ID NO:26.

SGK429 (SEQ ID NO:28) was amplified from the 48 cDNA panel source using the following oligonucleotide primers: 5′ primer: (SEQ ID NO: 173) GTTTCGGTTTCACCGCAGCCTCGCC. 3′ primer: (SEQ ID NO: 174) TGGAGAAGGAAAGAATGGGAGAAAC. Primer sequences were designed from partial or full-length versions of SEQ ID NO:28.

SGK093 (SEQ ID NO:3 1) was amplified from the 48 cDNA panel source using the following oligonucleotide primers: 5′ primer: (SEQ ID NO: 175) GAAAAGGAGGACATGGAGACCCAGG. 3′ primer: (SEQ ID NO: 176) CTTTACGCAGCTTCTCACGCTTTCG. Primer sequences were designed from partial or full-length versions of SEQ ID NO:31.

SGK087 (SEQ ID NO:33) was amplified from the 48 cDNA panel source using the following oligonucleotide primers: 5′ primer: (SEQ ID NO: 177) CCTAAAGCTTTTTAAGAACCAGCTG3′ primer: (SEQ ID NO: 178) CTGATGAATCCAAGCATGCTTGAGG. Primer sequences were designed from partial or full-length versions of SEQ ID NO:33.

SGK009 (SEQ ID NO:39) was amplified from the 48 cDNA panel source using the following oligonucleotide primers: 5′ primer: (SEQ ID NO: 179) GATTTTGGTCTGGCCAAGTGCAACG. 3′ primer: (SEQ ID NO: 180) GTATACATCGTGCTTGGTGTCGAAG. Primer sequences were designed from partial or full-length versions of SEQ ID NO:39.

SGK421 (SEQ ID NO:40) was amplified from the 48 cDNA panel source using the following oligonucleotide primers: 5′ primer: (SEQ ID NO: 181) AGGGCTCCTATGCAAAAGTAAAATC. 3′ primer: SEQ ID NO: 182) CCAGCAGTGGCTGAGGATCTCG. Primer sequences were designed from partial or full-length versions of SEQ ID NO:40.

SGK396 (SEQ ID NO:43) was amplified from the 48 cDNA panel source using the following oligonucleotide primers: 5′ primer: (SEQ ID NO: 183) GGAACGAGATCTTCTTGATGCTGAG. 3′ primer: (SEQ ID NO: 184) GGAAGGGCTGCTGCGAAGCTTTCTG. Primer sequences were designed from partial or full-length versions of SEQ ID NO:43.

SGK037 (SEQ ID NO:45) was amplified from the 48 cDNA panel source using the following oligonucleotide primers: 5′ primer: (SEQ ID NO: 185) GAATGGCAGGCTGTTTATTGTAATG. 3′ primer: (SEQ ID NO: 186) GGGAATAAGATTCTCTAAAAAGGGC. Primer sequences were designed from partial or full-length versions of SEQ ID NO:45.

SGK060 (SEQ ID NO:46) was amplified from the 48 cDNA panel source using the following oligonucleotide primers: 5′ primer: (SEQ ID NO: 187) CAGCTAAGTGTGTGAGTGGATCAAC; 3′ primer: (SEQ ID NO: 188) CCATCGTGATCAGCAACTGCTCTTC. Primer sequences were designed from partial or full-length versions of SEQ ID NO:46.

SGK080 (SEQ ID NO:47) was amplified from the 48 cDNA panel source using the following oligonucleotide primers: 5′ primer: (SEQ ID NO: 189) CTGAGAACTATGAAGTGTTGTACAC. 3′ primer: (SEQ ID NO: 190) CCAGGATCTGTCTGCTTTTTAGTTG. Primer sequences were designed from partial or full-length versions of SEQ ID NO:47.

SGK002 (SEQ ID NO:48) was amplified from the 48 cDNA panel source using the following oligonucleotide primers: 5′ primer: (SEQ ID NO: 191) GCCGGCGCTCACCATCAACCCTACC. 3′ primer: (SEQ ID NO: 192) CTCACTGAGCATCTTTAGGTCCGCC. Primer sequences were designed from partial or full-length versions of SEQ ID NO:48.

SGK058 (SEQ ID NO:49) was amplified from the 48 cDNA panel source using the following oligonucleotide primers: 5′ primer: (SEQ ID NO: 193) CCTATCCTATGGACCAAGGGTGAG. 3′ primer: (SEQ ID NO: 194) GTGCTCCGATGTAAAACATGGCGG. Primer sequences were designed from partial or full-length versions of SEQ ID NO:49.

SGK035 (SEQ ID NO:51) was amplified from the 48 cDNA panel source using the following oligonucleotide primers: 5′ primer: (SEQ ID NO: 195) GATGATGAAGAGACTGCCCTCCC. 3′ primer: (SEQ ID NO: 196) CATTGCTTCTTTAGCTGCCATGATC. Primer sequences were designed from partial or full-length versions of SEQ ID NO:51.

SGK075 (SEQ ID NO:52) was amplified from the 48 cDNA panel source using the following oligonucleotide primers: 5′ primer: (SEQ ID NO: 197) GATGCCATGCAGGGCCTTCAGC. 3′ primer: (SEQ ID NO: 198) CAAAGTCAACGAGCTTAACTCCTC. Primer sequences were designed from partial or full-length versions of SEQ ID NO:52.

Multiple Tissue Expression blots (MTE)

MTE (Multiple Tissue Expression) blots were obtained from Clontech Laboratories, Inc. These blots contained 84 arrayed cDNA samples derived from normal human tissue and human cell lines, and controls. The expression blots were prehybridized with ExpressHyb hybridization solution (Clontech Laboratories) containing 0.1 mg/ml denatured salmon sperm DNA at a temperature of 65° C. for two hours. Radioactive DNA probes were prepared using the Random Priming DNA labeling kit (Roche). Generation of DNA probe for SGK093 (Wnk3) Two synthetic oligonucleotides were designed which will amplify a 538 base pair fragment of Wnk3. The upstream oligonucleotide has the sequence (SEQ ID NO: 199) 5′ CCGCCACGCCAGCTACTCATCTAC 3′. The downstream oligonucleotide has the sequence (SEQ ID NO: 200) 5′ TCTCTTCAACAGGGTCTCCACTCG 3′. A fragment of the expected size was amplified by polymerase chain reaction from a human testes cDNA library and the sequence confirmed by DNA sequencing. Generation of DNA probe for SGKI 88 (EphA9): Two synthetic oligonucleotides were designed which will amplify a 387 base pair fragment of EphA9. The upstream oligonucleotide has the sequence (SEQ ID NO: 201) 5′ TTCTCCCAGATCCACAGC 3′. The downstream oligonucleotide has the sequence 5′ (SEQ ID NO: 202) ACCGGAGTCCTGCCTTGGAAG 3′. A fragment of the expected size was amplified by polymerase chain reaction from a human testes cDNA library and the sequence confirmed by DNA sequencing. Purified DNA fragments (100 ng) were labeled with 250 uCi of 32P-labeled dCTP for 45 minutes using the kit protocol. Unincorporated nucleotide was removed through the use of a spin column (ProbeQuant G50 micro columns, Amersham Pharmacia, Inc.). After denaturation by boiling for three minutes, the probe was introduced into the prehybridization solution, and the blot was hybridized at 65° C. for 20 hours. The blot was subsequently washed four times for 15 minutes each at 65° C. in a solution containing 15 mM NaCl, 1.5 mM Na₃Citrate, 0.1% sodium lauryl sulfate (SDS) and exposed to the phosphoimager screen for quantitation.

Northern Blot (SGK188)

Materials and Methods for the Northern blot

The northern blot was obtained from Clontech Laboratories, Inc. This blot contained 12 poly A+RNA samples from human tissues in the following order: whole brain, heart, skeletal muscle, colon, thymus, spleen, kidney, liver, small intestine, placenta, lung, peripheral blood leukocyte. The expression blots were prehybridized with ExpressHyb hybridization solution (Clontech Laboratories) containing 0.1 mg/ml denatured salmon sperm DNA at a temperature of 65° C. for two hours. A radioactive DNA probe was prepared using the Random Priming DNA labeling kit (Roche). Purified DNA fragment (100 ng) was labeled with 250 uCi of 32P-labeled dCTP for 45 minutes using the kit protocol. Unincorporated nucleotide was removed through the use of a spin column (ProbeQuant G50 micro columns, Amersham Pharmacia, Inc.). After denaturation by boiling for three minutes, the probe was introduced into the prehybridization solution, and the blot was hybridized at 65° C. for 20 hours. The blot was subsequently washed four times for 15 minutes each at 65° C. in a solution containing 15 mM NaCl, 1.5 mM Na₃Citrate, 0.1% sodium lauryl sulfate (SDS) and exposed to the phosphoimager screen for quantitation.

Analysis of the northern blot shows expression of SGK1 88 in human brain and colon. Two transcripts are detected of approximately 4.4 kb and 6 kb in size.

### Example 2e

### Classification of Polypeptides Exhibiting Kinase Activity among Defined Groups

AGC Group

Potential biological and clinical implications of the novel AGC group protein kinases are described next. SGK187 (SEQ ID NO:58) is a new DMPK family member that lists as its prototype myotonic dystrophy protein kinase (DMPK). Since the initial filing of this application, the murine orthologue of human SGK187 appeared in the public database as the full-length gene encoding Crik (AF086824) (J. Biol. Chem. 273 (45), 29706-29711 (1998)). Motif analysis of the amino acid sequence predicted for SGK187 (SEQ ID#58) showed the presence of four extracatalytic C-terminal domains: CNH, a plekstrin homology (PH) domain, a protein kinase C terminal domain and a phorbol ester/diacyl glycerol binding (C1) domain. Murine Crik is made as two different isoforms, a long 240-kDa protein in which the kinase domain is followed by the sequence for Citron, a Rho/Rac-binding protein comprising the CNH, PH and C1 domains. The second form of Crik is a short 54-kDa protein (CRIK-short kinase (SK)) lacking the Citron sequence. In keratinocytes, full-length CRIK, but not CRIK-SK, localizes into corpuscular cytoplasmic structures and recruits actin into these structures. Like the ubiquitously expressed Rho-associated kinases ROCK I and II kinases, Crik may participate in the dynamics of the actin cytoskeleton, albeit in a more tissue-restricted manner.

SGK064 (SEQ ID NO:59) is a new GRK family member that lists as its prototype the G protein-coupled receptor kinases (i.e. beta ARK1). Domain analysis of SGK064 revealed the presence of an RGS domain. The RGS (regulator of G protein signaling) domain (PF00615) is approximately 125 amino acids long and found in family members that include the GTPase-activating proteins for heterotrimeric G-protein alpha-subunits. G protein-coupled receptor kinases function in the beta-adrenergic signaling cascade, an important regulator of myocardial function. Several cardiovascular diseases such as congestive heart failure display alterations in this pathway (Proc Assoc Am Physicians September-October 1999;111(5):399-405). The predicted open reading frame for SGK064 is 84% identical to GRK7 isolated from Spermophilus tridecemlineatus (ground squirrel). From this observation, SGK064 may represent the human orthologue or a very close homolog of Spermophilus tridecemlineatus GRK7. GRK7 may play an important role in signal transduction in the cones and rods of the visual system. Desensitization in the rod cell of the mammalian retina is initiated when light-activated rhodopsin is phosphorylated by the G protein-coupled receptor kinase (GRK) or GRK1. Like GRK1, GRK7 may function as an opsin kinase in the mammalian signal phototransduction process.

SGK409 (SEQ ID NO:60) is a new MAST family member that list as its prototype the microtubule-associated testis specific serine/threonine protein (MAST205 ). Domain analysis of SGK409 revealed the presence of a PDZ domain. The PDZ domain (of approximately 83 amino acids in length) is found in membrane-associated proteins that include homologues of the MAGUK family of guanylate kinases, several protein phosphatases and protein kinases. PDZ domains are also found in neuronal nitric oxide synthase as well as in the subfamily of dystrophin-associated proteins, collectively known as syntrophins. SGK409 is 60% identical (over 1351 amino acids) to murine MAST205, a protein implicated in mammalian spermiogenesis (Biol Reprod. November 1996;55(5):1039-44). MAST205 and the related SAST (syntrophin-associated serine/threonine kinase) associate with beta 2-syntrophin via PDZ-PDZ domain interactions. MAST205 colocalizes with beta 2-syntrophin and utrophin at neuromuscular junctions. (Nat Neurosci July 1999;2(7):611-7). Like MAST205 and SAST, SGK409 may be an essential player at the neuromuscular junction by linking the dystrophin/utrophin network to microtubules via the syntrophins.

SGK021 (SEQ ID NO:61) is a new member Mo3C11.1_ce family member named after the c. elegans Mo3C11 hypothetical open reading frame. One Mo3C11.1 family member, Tank-binding kinase (TBK1), has been studied with respect to its signaling properties. TBK1 is an IKK-related kinase capable of activating the NfkB pathway following its interaction with the adaptor molecules TRAF2 and TANK (EMBO J Dec. 1, 1999;18(23):6694-704). SGK021 may share signaling properties with TBK1.

SGK410 (SEQ ID NO:62) is a new PKC family member closest to PKCiota (95% identity over 587 amino acids). PKCiota has been implicated to function in the cancer cell survival pathway since it protects human leukemia cells against drug-induced apoptosis (J Biol Chem Oct. 31, 1997;272(44):27521-4, J Biol Chem Feb. 12, 1999;274(7):3927-30). Given the high degree of homology between SGK410 and PKCiota, it is conceivable that SGK410 functions in the cell survival pathway in cancer.

SGK069 (SEQ ID NO:63) and SGK110 (SEQ ID NO:64) are new AGC group members that do not cluster with other AGC kinases, hence are referred to as unique family members. The low degree of homology of SGK069 and SGK110 to their closest kinases (42 and 40%) precludes making any homology-based inferences on the potential signaling properties of these novel AGC group kinases. The 48 tissue PCR-based expression pattern for SGK069 showed it to be restricted both in normal (confined to skeletal muscle) and tumor tissues (kidney carcinoma). The same PCR-based expression panel for SGK110 showed this gene to be expressed in placenta, spleen, thyroid gland, uterus and lung. SGK100 was also expressed in multiple cancer cell lines (see, e.g., Table 7).

CAMK Group

Potential biological and clinical implications of the novel CAMK group protein kinases are described next. SGK053 (SEQ ID NO:65) and SGK124 (SEQ ID NO:66) are novel AMPK family members of the CAMK group. Since the initial filing of this application, the full-length version of human SGK053 appeared in the public database as CamKI-like protein kinase (CaMKI) (NP_{—}065130.1 ). CaMKI is a calcium-calmodulin-dependent kinase expressed in granulocytes whose transcription is upregulated during neutrophilic differentiation of CD34(+) stem cells. Activation of CaMKI induces extracellular signal-related kinase (ERK) mitogen-activated protein (MAP) kinase activity and CRE-binding protein (CREB) transcriptional activity (Blood. 2000;96:3215-3223). Since the initial filing of this application, the full-length version of human SGK124 appeared in the public database (CAB81634.1) as a hypothetical protein of unknown function.

SGK254 (SEQ ID NO:67), SGK297 (SEQ ID NO:68) and SGK411 (SEQ ID NO:69) are novel CAMK family members of the CAMK group. SGK254 is most likely the human orthologue or a very close homolog of rat Ca2+/calmodulin-dependent PK IV (CaM-kinase IV) (91% over 505 amino acids). CaM-kinase IV mediates Ca(2+)-dependent regulation through the phosphorylation of specific transcription factors in the central nervous and immune systems. CaM-kinase I and IV but not CaM-kinase II are activated by an upstream CaM-kinase kinase (J Biol Chem Aug. 18, 1995;270(33):19320-4). Since the initial filing of this application, the full-length versions of human SGK297 and SGK 411 appeared in the public database as Ca2+/Calmodulin-dependent protein kinase I (CamKI)-like protein kinase (CaMKI (AAF74509.1) and multifunctional CAMK II delta2 (AAD20442.1), respectively. SGK297 was identified as a hypothetical ORF from genomic sequence from Xq28 (Genomics Aug. 15, 1997;44(1):8-14). The putative rat orthologue of human SGK297, rat Ca2+/calmodulin (CaM)-dependent protein kinase I exists in at least three splicing isoforms (CaM kinase, Ibeta1 and Igamma) that provide alternative means of Ca2+/Calmodulin-dependent signaling in the nervous system (J Biol Chem Dec. 19, 1997;272(51):32704-8 ). SGK 411 encodes multifunctional CAMK II delta2, a kinase potentially implicated in heart failure (Circ Res Apr. 2, 1997;84(6):713-21).

SGK004 (SEQ ID NO:75), SGK006 (SEQ ID NO:76) and SGK180(SEQ ID NO:77) are novel CAMK group members belonging to the SNF1 subfamily of EMK-1 kinases. SGK386 (SEQ ID NO:78) is a novel CAMK group member belonging to the MLCK family.

Since the initial filing of this application, SGK004 and SGK180 appeared in the public database as the full-length gene encoding SNF1LK (P57059) and HSNFRK (AAF86944.1), respectively. SGK006 is 46% identical over 121 amino acids to mouse hormonally upregulated Neu-associated kinase (Hunk or MAK-V) (NP_{—}056570.1 ), a hypothetical protein predicted from the entire sequence of human chromosome 21 (Nature 405 (6784), 311-319 (2000)). SGK004, SGK006 and SGK180 display homology to human Hunk (NP056570). These three kinases also display significant homology to rat salt-induced kinase (SIK, also known as KID2 protein) (NP_{—}067725.1), mouse myocardial SNF1-like kinase (MSK)( NP_{—}034961.1) and chicken Qik (qin-induced kinase)( AAF28351). From yeast to plants and mammals, the SNF1 protein kinase plays a central role in stress-response pathways (Annu Rev Biochem 1998;67:821-55). The ubiquitously expressed Qik protein may play a role in oncogenesis (Biochem Biophys Res Commun Sep. 24, 2000;276(2):564-70Z) and Hunk (MAK-V) may have a role in pregnancy and mammary carcinogenesis (Development October 2000;127(20):4493-509). SGK004, SGK006 and SGK180 may fulfill roles in the response to cellular stress. Disruptions in the signaling pathways in which any of these novel kinases participate may trigger cancer or other disease conditions.

SGK386 (SEQ ID NO:78) is another CAMK group member presented in this application. Since the initial filing of this application, SGK386 appeared in the public database as the full-length gene encoding myosin, light polypeptide kinase (MLCK) (CAC10006.1). MLCK is responsible for regulating the contractility of actomyosin fibers, both in smooth muscle as well as in non-muscle vertebrate cells. MLCK has been implicated in cell growth regulation through its ability to modulate myosin II motor activity via protein phosphorylation, thereby triggering cytokinesis (J Cell Biol Oct. 30, 2000;151(3):697-708). Disruptions in SGK386 signaling may also be involved in triggering cancer or other disease conditions.

Casein Kinase Group

SGK003 (SEQ ID NO:79) and SGK006 (SEQ ID NO:80) are novel casein kinases that belong to the CKI family. SGK003 is 91% identical over 304 amino acids to casein kinase I, alpha 1. The high degree of sequence homology between SGK003 and CKI, alpha suggests that these two kinases may have overlapping functions in processes related to vesicular trafficking, DNA repair, cell cycle progression or cytokinesis. Noteworthy is the elevated expression levels observed for SGK003 in tumor versus normal tissue sources as determined from both, the tissue array and PCR-based expression studies. SGK003 was found to be expressed in the following normal sources: spinal cord, uterus, fetal brain, fetal kidney, fetal lung and adrenal gland. SGK003 was overexpressed in tumors associated with the following tissues: colon, lung, prostate, ovary, bone, breast and brain (glioblastoma). SGK003, therefore, may be a potential target for therapeutical intervention in oncology. SGK006, the second novel CKI family member described in this application is 53% identical over 166 amino acids to the hypothetical open reading frame R90.1 from c. elegans.

CMGC Kinase Group

SGK041 (SEQ ID NO:81) and SGK112 (SEQ ID NO:82) are novel CMGC protein kinases that belong to the CDK family. Since the initial filing of this application, SGK041 appeared in the public database as the full-length gene encoding Nkiamre (NP_{—}057592.1). Nkiamre is the human orthologue or a very close homolog of the rat protein Nkiatre. Nkiamre was found to be deleted at both alleles in 9 of 18 leukemic samples with chromosome band 5q31 abnormalities revealed by fluorescence in situ chromosomal hybridization (FISH) (Cancer Res Aug. 15, 1999;59(16):4069-74). SGK041 may have tumor suppressor activities required for normal hematopoiesis. Loss of SGK041 and/or deregulation of its signalling pathway may be implicated in the etiology of human acute leukemia and myelodysplasia (Cancer Res Aug. 15, 1999;59(16):4069-74). SGK112, the second novel CDK family member in this application is 61% related to the human CDC2-related kinase (CCRK) whose function is unknown. On the basis of sequence similarity to cyclin-dependent kinase 2 (Cdk2 (44% amino acid identity over 480 amino acids), SGK112 may have cell cycle regulatory functions akin to those played by Cdk2. Cdk2 has been implicated in the etiology of non-small cell lung carcinoma (Cell Growth Differ October 2000;11(10):507-15). Disruptions in SGK112 signaling may also be involved in triggering cancer or other disease conditions.

SGK038 (SEQ ID NO:83), is most likely the human orthologue or a very close homolog of rat ERK7 (P42525). ERK7 is a nuclear-localized, constitutively active MAP kinase capable of inhibiting cellular growth. In spite of the presence of the canonical TEY catalytic motif found in other MAP kinases, ERK7 is not responsive to the mitogenic stimuli that normally activate the MAP kinase pathways. The kinase activity, growth-inhibitory and nuclear localization properties of ERK7 are dependent on an intact C-terminal extracatalytic region (Mol Cell Biol February 1999;19(2):1301-12). The C-terminus of ERK7 has been shown to bind through its C-terminal region to an intracellular chloride ion (CLIC3). (J Biol Chem Jan. 15, 1999;274(3):1621-7). Disruptions in SGK038 signaling may also be involved in triggering cancer or other disease conditions.

Microbial PK Group

SGK158 (SEQ ID NO:84) and SGK429 (SEQ ID NO:85) are new members of the ATP binding cassette transporter (ABC) family of mammalian microbial-like kinases. Since the initial filing of this application, SGK158 and SGK429 appeared in the public database as the full-length gene encoding two hypothetical proteins predicted from genomic sequence (NP_{—}064632 and AAD43192.1, respectively). One mammalian ABC protein, ABC1 has been studied with respect to its physiological role. ABC1 is required for cell engulfment by macrophages undergoing apoptosis. Other members of the ABC1 family of mammalian proteins include the cystic fibrosis transmembrane conductance (CFTR) regulator and as P-glycoprotein. Like CFTR and P-glycoprotein, ABC1 is an anion transporter. In microbes, ABC transporter play an important role in multidrug resistance (Biochim Biophys Acta Jun. 10, 1998;1365(1-2):31-6). Like other ABC protein, SGK158 and SGK429 may have a function in the mechanism of drug resistance.

SGK152 (SEQ ID NO:86), the third novel microbial-like kinase, belongs to the R101 family. Since the initial filing of this application, SGK152 appeared in the public database as the full-length gene encoding the human homolog of Aspergillus nidulans sudD (suppressor of bimD6) (NP_{—}003822.1). sudD is conserved from yeast to mammals and appears to play a key function in cell cycle regulation related to chromosome condensantion and segregation (Gene May 12, 1998;211(2):323-9). Disruptions in SGK152 signaling may also be involved in triggering cancer or other disease conditions.

“Other” Group

SGK077 (SEQ ID NO:87), a novel C26C2 family member, is closest to murine hapsin (BAB00640.1) (62% amino acid identity over 492 amino acids). Both SGK077 and hapsin have motifs predicted from a hidden Markov model analysis that suggests that these proteins act as kinases. The biological function of hapsin is unknown.

SGK093 (Wnk3) (SEQ ID NO:88), a novel C26C2 family member, is closest to kinase-deficient protein (KDP) (AAF31483.1) (60% amino acid identity over 321 amino acids). Wnk3 is a member of a subfamily of serine/threonine kinases which includes a described prototype, Wnk1, isolated from rat (J Biol Chem Jun. 2, 2000;275(22):16795-801). This family is characterized by an N-terminal catalytic domain with several unique sequence features, most notably a change of the invariant lysine in kinase subdomain II to a cysteine, coupled with a change of the third conserved glycine residue in subdomain I into a lysine. The resulting enzyme appears to maintain catalytic activity due to this concomitant switch. Wnk3 conserves both of these catalytic changes and therefore is predicted to maintain catalytic activity. The long C-terminal portion of the Wnks includes many protein interaction domains such as SH3 binding sites and coiled coil regions.

The Wnk family catalytic domain shows the highest similarity to two families of serine/threonine kinases: The MEKK-like kinases and the Ste20-like kinases. Both of these families can regulate enzymes in various MAPK signaling cascades, which are critical for many cellular processes such as mitogenesis, differentiation, cell survival, and stress response. The Ste20 kinases are also involved in regulation of the ras/rac/rho/cdc42 pathways and subsequent downstream effects on cytoskeleton. Wnk3 shows high expression in human kidney, in kidney carcinoma cell lines, in prostate, prostate cell lines, and prostate tumor bone metastases, in colorectal tissue and tumor cell lines, and in human leukemia cells. Therefore Wnk3 may be involved in the normal homeostasis and functioning of the human kidney, prostate, and digestive system, and may be involved in tumorigenesis which arises from these three tissues. High expression in human leukemia cell lines indicates a possible role in the development of that disease as well.

The partial SGK074 (SEQ ID NO:89), a novel Dyrk family member, is closest to the murine Myak protein kinase (AAD41593.1) with 50% amino acid sequence identity over 176 amino acids. Murine Myak is the homolog of the yeast kinase Yak1. The Yak1 family of related genes includes drosophila (Mnb) and human minibrain (Dyrk1a) and the rat steroid hormone receptor interacting protein ANPK. Myak features a nuclear localization signal and PEST motifs flanking its catalytic domain (Mol Reprod Dev April 2000;55(4):372-8). SGK074 may represent an additional member of steroid-responsive protein kinases.

SGK087 (SEQ ID NO:90) and SGK295, Kis, P-CIP2 (SEQ ID NO:91) represent the two additional Dyrk family members in this application. Since the initial filing of this application, SGK087 and SGK295 appeared in the public database as the full-length gene Dyrk4 (AAF91393.1) (predicted from genomic sequence) and the rat Kis protein kinase (Kinase interacting with stathmin (P-CIP2) (NP_{—}058989.1). Dyrk is the fourth member of the human Dyrk family of protein kinases, the other three being Dyrk1 (human minibrain), Dyrk2 and Dyrk3. The functional role of Dyrk's is presently unknown. P-CIP2 was found to be an interacting protein for the cytosolic COOH-terminal domain (CD) of peptidylglycine alpha-amidating monooxygenase (PAM), a key element in the secretory pathway of neurons and endocrine cells (J Biol Chem Nov. 8, 1996;271(45):28636-40).

SGK419 (SEQ ID NO:92) represents a novel NAK family member within the Other group. Since the initial filing of this application, SGK0419 appeared in the public database as the full-length gene encoding the hypothetical protein CAB70863.1 predicted from human genomic sequencing. SGK419 is closely related to drosophila Numb-associated kinase (NAK) (65% sequence identity over 294 amino acids). In drosophila, NAK, through its interaction with the membrane-associated protein Numb, participates in cell fate determination during asymmetric cell divisions (Mol Cell Biol January 1998;18(1):598-607). SGK419 may play a similar role as NAK in processes related to cell differentiation and proliferation.

SGK125, Myo3A (SEQ ID NO:93) represents a novel NinaC family kinase within the Other group. Since the initial filing of this application, SGK125 appeared in the public database as the full-length gene encoding MyoIIIA (NP_{—}059129). SGK125 displays 34% sequence identity over 1155 amino acids to drosophila Ninac, another class III myosin. Like drosophila NinaC, Myo3A displays a myosin head domain as well as 3 calmodulin-binding (IQ) domains. Ninac plays a key role in the photoreceptor light signal transduction process (Biochim Biophy Acta Mar. 17, 2000;1496(1):52-9. SGK125 may represent a functional counterpart of Ninac with roles in mammalian phototransduction.

The partial SGK445 (SEQ ID NO:94) represents a novel polo kinase (PLK) family kinase within the Other group. SGK445 is closest to murine Sak (AAC37649.1), a drosophila polo-like kinase, with 65% sequence identity over 47 amino acids. The close homology of SGK445 to Sak suggests that SGK445 may represent the human orthologue of the murine kinase. Sak expression is associated with the processes of mitosis and meiosis. The fruit fly and mammalian forms of PLK have been shown to play a key role in cell cycle progression (Curr Opin Cell Biol December 1998;10(6):776-83). SGK445, like Sak and PLK, may play similar roles during cell proliferation.

SGK127 (SEQ ID#95) represents a novel RAF family kinase within the Other group.

SGK127 is closest to KSR (kinase suppressor of Ras) NP_{—}038599.1 with 47% sequence identity over 446 amino acids. KSR has been shown to function in the Ras-mediated signal transduction of multiple receptor tyrosine kinase- activated pathways. Hence, SGK127 may be implicated in cell growth regulation. Disruptions in SGK127 signaling may be involved in triggering cancer or other disease conditions

SGK009, Ankrd3 (SEQ ID NO:96) represents a novel RIP family kinase within the Other group. Since the initial filing of this application, SGK009 appeared in the public database as the full-length gene encoding Ankrd3 (NP_{—}065690.1) predicted from human genomic sequence from chromosome 21 (Nature May 18, 2000;405(6784):311). SGK009 features 10 extracatalytic C-terminal ankyrin domains. The biological function of Ankrd3 is unknown. The presence of multiple ankyrin domains in SGK009 (Ankrd3) suggests that this protein plays an important scaffolding role akin to that observed in the integrin-like kinases (Int J Mol Med June 1999;3(6):563-72). Such scaffolding kinases participate in integrin-, growth factor- and Wnt-signaling pathways that are important in normal as well as tumor cell proliferation.

SGK421, STK22A, TSK1 (SEQ ID NO:97) represents a novel STK22A family kinase within the Other group. Since the initial filing of this application, the nucleic sequence corresponding to SGK421 appeared in the public database from genomic sequence (AC008476). Based on sequence homology, SGK421 appears to be the human orthologue of the murine kinase STK22A (NP_{—}033461.1). TSK1 maps to the DiGeorge critical region (DGCR) human chromosome 22q11 and is syntenic with the STK22A gene in the mouse genome (chromosome 16). Patients with DiGeorge syndrome have velocardiofacial or conotruncal facial anomalies (J Otolaryngol September-October 2000;21(5):326-30). SGK421 may be implicated in the etiology of DiGeorge syndrome.

The partial SGK047 (SEQ ID NO:98) represents a novel STKR family kinase within the Other group. SGK047 is closest to bone morphogenetic protein (BMP) receptor, type IA (NP_{—}004320.1) with 46% sequence identity over 11 amino acids. The full-length form of SGK047 may potentially encode a novel bone morphogenetic protein receptor. BMP receptors play a key role in embryogenesis (Cell Mol Life Sci. June 2000;57(6):943-56.).

SGK196 (SEQ ID NO:99) represents a novel unique family kinase within the Other group. Since the initial filing of this application, SGK196 appeared in the public database as the full-length gene encoding the hypothetical ORF BAB15623.1. SGK196 is closest to multiple protein kinases from Arabidopsis thaliana sharing 28% sequence identity over 153 amino acids. The function of the plant kinases is unknown.

SGK396 (SEQ ID NO:100) represents a novel unique family kinase within the Other group. SGK396 is closest to the Arabidopsis thaliana kinase (BAB 11570.1) with 28% amino acid sequence identity over 34 amino acids. The function of the plant kinase is unknown

SGK279, PKN (SEQ ID NO:101) represents a novel YWY3_ce family kinase within the Other group. SGK279 is closest to the rat kinase encoded by BAA36362.1 with 94% sequence identity over 400 amino acids. The function of the rat kinase is unknown.

The STE Group

SGK037 (SEQ ID NO:102), SGK060 (SEQ ID NO:103) and SGK080 (SEQ ID NO:104) represent three novel NEK family members of the STE group. SGK037 is closest to NEK1 with 65% sequence identity over 176 amino acids. Since the initial filing of this application, SGK060 appeared in the public database as the full-length gene encoding the hypothetical protein hypothetical BAB15672.1. SGK080 is closest to NRK (NP_{—}002488.1) with 90% sequence identity over 404 amino acids. NEK family kinases such as NED1 and NRK are related to the mitotic regulator NimA from Aspergillus nidulans. Given the homology of SGK037, SGK060 and SGK080 to various NEK family members, these novel kinases may partticipate in cell cycle regulation. Disruptions in the signaling pathways in which SGK037, SGK060 and SGK080 participate may be associated with cancer or other diseases.

SGK002 (SEQ ID NO:105), SGK058 (SEQ ID NO:106) and SGK103 (SEQ ID NO:107) represent three novel STE11 family members of the STE group. SGK002 (SEQ ID NO:105) is closest to MEK2 (P36507) with 92% sequence identity over 369 amino acids. A second MEK-like kinase is represented by SGK058. Since the initial filing of this application, SGK058 appeared in the public database as the full-length gene encoding the hypothetical protein BAB15538.1. Given the high degree of similarity between SGK002 and MEK2, SGK002 may be implicated in MAP kinase pathway regulation.

The partial SGK103 (SEQ ID NO:107) represents the third STE1 1 family kinase of this application. SGK103 is closest to drosophila fused (CAA39285.1) with 57% sequence homology over 16 amino acids.

SGK035 (SEQ ID NO:108) and SGK075 (SEQ ID NO:109) represent two novel STE20 family members of the STE group. SGK035 display 92% over 318 amino acids to PAK2 Q13177. SGK075 displays 67% over 227 amino acids to myosin IIIA. The STE20 family of protein kinases represent key membrane-proximal regulators of multiple signal transduction pathways important in cell proliferation, survival, differentiation and response to cellular stress, all potential functions for the polypeptides represented by SGK035 and SGK075.

The TK Group

SGK188 (SEQ ID NO:110) is a novel receptor tyrosine kinase belonging to the EphA9 family. The Eph family is the largest subfamily of receptor tyrosine kinases in the human genome. This family has a stereotyped structure consisting of an N-terminal globular domain involved in ligand binding, two Type III fibronectin-like domains which contribute to receptor dimerization, a transmembrane domain, and an intracellular tyrosine kinase domain.

SGK040 (SEQ ID NO:54), the second novel member of the tyrosine kinase group, belongs to the unique family since it fails to cluster with other tyrosine kinase family members. SGK040 displays 30% sequence identity over 76 amino acids to the rat cytoplasmic tyrosine kinase (CTK) BATK (P41243). Given the pivotal role of CTK's in signal transduction (Int J Mol Med. June 2000;5(6):583-90, Curr Pharm Des. March 2000;6(4):361-78), disruptions in SGK040 signaling may be involved in triggering cancer or other disease conditions.

### Example 2f

### Classification of Polypeptides Exhibiting Kinase-Like Activity Among Defined Groups

DAG Kinase Group

SGK390 (SEQ ID NO:112) represents a novel family member of the DAG family of kinases. Since the initial filing of this application, a potential orthologue or very close homolog from Mesocricetus auratus (hamster) of human SGK390 appeared in the public database as the full-length gene encoding diacylglycerol kinase eta (DAG kinase eta) (NP_{—}057592.1). Like its hamster counterpart, SGK390 contains multiple extracatalytic domains defined from a profile analsysis, including a diacylglycerol/phorbol ester binding domain, a plekstrin domain and a diacylglycerol kinase accesory domain. Hamster DAG kinase eta was shown to have a wide tissue distribution in contrast to other DAG kinases which display a more restricted tissue distribution pattern. DAG kinases have been shown to play a key role in regulating the concentration of the seccond messenger DAG (J Biol Chem Aug. 16, 1996;271(33):19781-8). Given the potential role of SGK390 in regulating DAG levels, disruptions in the signaling pathway in which this kinase participates may trigger cancer or other disease conditions.

Guanylate Cyclases (GCyc) Group

SGK007 (SEQ ID NO:113) and SGK050 (SEQ ID NO:114) are novel members of the guanylate cyclase family of protein kinase-like molecules. SGK007 is 59% identical over 499 amino acids to rat guanylate cyclase; hence, SGK007 is the human homologue or a close homolog of the rat protein. The partial SGK050 polypeptide is 48% identical over 23 amino acids to the natriuretic peptide receptor B precursor, isoform b (NP_{—}003986.2). A similar homology is found between SGK050 and guanylate cyclase from multiple species.

### Example 3

### Isolation of cDNAs Encoding Mammalian Protein Kinases

Materials and Methods

Identification of Novel Clones

Total RNAs are isolated using the Guanidine Salts/Phenol extraction protocol of Chomczynski and Sacchi (P. Chomczynski and N. Sacchi, Anal. Biochem. 162, 156 (1987)) from primary human tumors, normal and tumor cell lines, normal human tissues, and sorted human hematopoietic cells. These RNAs are used to generate single-stranded cDNA using the Superscript Preamplification System (GIBCO BRL, Gaithersburg, Md.; Gerard, G F et al. (1989), FOCUS 11, 66) under conditions recommended by the manufacturer. A typical reaction uses 10 μg total RNA with 1.5 μg oligo(dT)₁₂₋₁₈ in a reaction volume of 60 μL. The product is treated with RNaseH and diluted to 100 μL with H₂O. For subsequent PCR amplification, 1-4 μL of this sscDNA is used in each reaction.

Degenerate oligonucleotides are synthesized on an Applied Biosystems 3948 DNA synthesizer using established phosphoramidite chemistry, precipitated with ethanol and used unpurified for PCR. These primers are derived from the sense and antisense strands of conserved motifs within the catalytic domain of several protein kinases. Degenerate nucleotide residue designations are: N=A, C, G, or T; R=A or G; Y═C or T; H=A, C or T not G; D=A, G or T not C; S═C or G; and W=A or T.

PCR reactions are performed using degenerate primers applied to multiple single-stranded cDNAs. The primers are added at a final concentration of 5 μM each to a mixture containing 10 mM TrisHCl, pH 8.3, 50 mM KCl, 1.5 mM MgCl₂, 200 μM each deoxynucleoside triphosphate, 0.001% gelatin, 1.5 U AmpliTaq DNA Polymerase (Perkin-Elmer/Cetus), and 1-4 μL cDNA. Following 3 min denaturation at 95° C., the cycling conditions are 94° C. for 30 s, 50° C. for 1 min, and 72° C. for 1 min 45 s for 35 cycles. PCR fragments migrating between 300-350 bp are isolated from 2% agarose gels using the GeneClean Kit (Bio101), and T-A cloned into the pCRII vector (Invitrogen Corp. U.S.A.) according to the manufacturer's protocol.

Colonies are selected for mini plasmid DNA-preparations using Qiagen columns and the plasmid DNA is sequenced using a cycle sequencing dye-terminator kit with AmpliTaq DNA Polymerase, FS (ABI, Foster City, Calif.). Sequencing reaction products are run on an ABI Prism 377 DNA Sequencer, and analyzed using the BLAST alignment algorithm (Altschul, S. F. et al., J. Mol. Biol. 215: 403-10).

Additional PCR strategies are employed to connect various PCR fragments or ESTs using exact or near exact oligonucleotide primers. PCR conditions are as described above except the annealing temperatures are calculated for each oligo pair using the formula: Tm=4(G+C)+2(A+T).

Isolation of cDNA Clones:

Human cDNA libraries are probed with PCR or EST fragments corresponding to kinase-related genes. Probes are ³²P-labeled by random priming and used at 2×10⁶ cpm/mL following standard techniques for library screening. Pre-hybridization (3 h) and hybridization (overnight) are conducted at 42 oC in 5×SSC, 5× Denhart's solution, 2.5% dextran sulfate, 50 mM Na₂PO₄/NaHPO₄, pH 7.0, 50% formamide with 100 mg/mL denatured salmon sperm DNA. Stringent washes are performed at 65° C. in 0.1×SSC and 0.1% SDS. DNA sequencing was carried out on both strands using a cycle sequencing dye-terminator kit with AmpliTaq DNA Polymerase, FS (ABI, Foster City, Calif.). Sequencing reaction products are run on an ABI Prism 377 DNA Sequencer.

### Example 4

### Expression Analysis of Mammalian Protein Kinases

Materials and Methods

Northern Blot Analysis

Northern blots are prepared by running 10 μg total RNA isolated from 60 human tumor cell lines (such as HOP-92, EKVX, NCI-H23, NCI-H226, NCI-H322M, NCI-H460, NCI-H522, A549, HOP-62, OVCAR-3, OVCAR-4, OVCAR-5, OVCAR-8, IGROV1, SK-OV-3, SNB-19, SNB-75, U251, SF-268, SF-295, SF-539, CCRF-CEM, K-562, MOLT-4, HL-60, RPMI 8226, SR, DU-145, PC-3, HT-29, HCC-2998, HCT-116, SW620, Colo 205, HTC15, KM-12, UO-31, SN12C, A498, CaKil, RXF-393, ACHN, 786-0, TK-10, LOX IMVI, Malme-3M, SK-MEL-2, SK-MEL-5, SK-MEL-28, UACC-62, UACC-257, M14, MCF-7, MCF-7/ADR RES, Hs578T, MDA-MB-231, MDA-MB-435, MDA-N, BT-549, T47D), from human adult tissues (such as thymus, lung, duodenum, colon, testis, brain, cerebellum, cortex, salivary gland, liver, pancreas, kidney, spleen, stomach, uterus, prostate, skeletal muscle, placenta, mammary gland, bladder, lymph node, adipose tissue), and 2 human fetal normal tissues (fetal liver, fetal brain ), on a denaturing formaldehyde 1.2% agarose gel and transferring to nylon membranes.

Filters are hybridized with random primed [α³²P]dCTP-labeled probes synthesized from the inserts of several of the kinase genes. Hybridization is performed at 42° C. overnight in 6×SSC, 0.1% SDS, 1× Denhardt's solution, 100 μg/mL denatured herring sperm DNA with 1-2×10⁶ cpm/mL of ³²P-labeled DNA probes. The filters are washed in 0.1×SSC/0.1% SDS, 65° C., and exposed on a Molecular Dynamics phosphorimager.

Quantitative PCR Analysis

RNA is isolated from a variety of normal human tissues and cell lines. Single stranded cDNA is synthesized from 10 μg of each RNA as described above using the Superscript Preamplification System (GibcoBRL). These single strand templates are then used in a 25 cycle PCR reaction with primers specific to each clone. Reaction products are electrophoresed on 2% agarose gels, stained with ethidium bromide and photographed on a UV light box. The relative intensity of the STK-specific bands were estimated for each sample.

DNA Array Based Expression Analysis

Plasmid DNA array blots are prepared by loading 0.5 μg denatured plasmid for each kinase on a nylon membrane. The [γ³²P]dCTP labeled single stranded DNA probes are synthesized from the total RNA isolated from several human immune tissue sources or tumor cells (such as thymus, dendrocytes, mast cells, monocytes, B cells (primary, Jurkat, RPM18226, SR), T cells (CD8/CD4+, TH1, TH2, CEM, MOLT4), K562 (megakaryocytes). Hybridization is performed at 42° C. for 16 hours in 6×SSC, 0.1% SDS, 1× Denhardt's solution, 100 μg/mL denatured herring sperm DNA with 10⁶ cpm/mL of [γ³²P]dCTP labeled single stranded probe. The filters are washed in 0.1×SSC/0.1% SDS, 65° C., and exposed for quantitative analysis on a Molecular Dynamics phosphorimager.

### Example 5

### Protein Kinase Gene Expression

Vector Construction

Materials and Methods

Expression Vector Construction

Expression constructs are generated for some of the human cDNAs including: a) full-length clones in a pCDNA expression vector; b) a GST-fusion construct containing the catalytic domain of the novel kinase fused to the C-terminal end of a GST expression cassette; and c) a full-length clone containing a Lys to Ala (K to A) mutation at the predicted ATP binding site within the kinase domain, inserted in the pCDNA vector.

The “K to A” mutants of the kinase might function as dominant negative constructs, and will be used to elucidate the function of these novel STKs.

### Example 6

### Generation of Specific Immunoreagents to Protein Kinases

Materials and Methods

Specific immunoreagents are raised in rabbits against KLH- or MAP-conjugated synthetic peptides corresponding to isolated kinase polypeptides. C-terminal peptides were conjugated to KLH with glutaraldehyde, leaving a free C-terminus. Internal peptides were MAP-conjugated with a blocked N-terminus. Additional immunoreagents can also be generated by immunizing rabbits with the bacterially expressed GST-fusion proteins containing the cytoplasmic domains of each novel PTK or STK.

The various immune sera are first tested for reactivity and selectivity to recombinant protein, prior to testing for endogenous sources.

Western blots

Proteins in SDS PAGE are transferred to immobilon membrane. The washing buffer is PBST (standard phosphate-buffered saline pH 7.4+0.1% Triton X-100). Blocking and antibody incubation buffer is PBST+5% milk. Antibody dilutions varied from 1:1000 to 1:2000.

### Example 7

### Recombinant Expression and Biological Assays for Protein Kinases

Materials and Methods

Transient Expression of Kinases in Mammalian Cells

The pcDNA expression plasmids (10 μg DNA/100 mm plate) containing the kinase constructs are introduced into 293 cells with lipofectamine (Gibco BRL). After 72 hours, the cells are harvested in 0.5 mL solubilization buffer (20 mM HEPES, pH 7.35, 150 mM NaCl, 10% glycerol, 1% Triton X-100, 1.5 mM MgCl₂, 1 mM EGTA, 2 mM phenylmethylsulfonyl fluoride, 1 μg/mL aprotinin). Sample aliquots are resolved by SDS polyacrylamide gel electrophoresis (PAGE) on 6% acrylamide/0.5% bis-acrylamide gels and electrophoretically transferred to nitrocellulose. Non-specific binding is blocked by preincubating blots in Blotto (phosphate buffered saline containing 5% w/v non-fat dried milk and 0.2% v/v nonidet P-40 (Sigma)), and recombinant protein was detected using the various anti-peptide or anti-GST-fusion specific antisera.

In Vitro Kinase Assays

Three days after transfection with the kinase expression constructs, a 10 cm plate of 293 cells is washed with PBS and solubilized on ice with 2 mL PBSTDS containing phosphatase inhibitors (10 mM NaHPO₄, pH 7.25, 150 mM NaCl, 1% Triton X-100, 0.5% deoxycholate, 0.1% SDS, 0.2% sodium azide, 1 mM NaF, 1 mM EGTA, 4 mM sodium orthovanadate, 1% aprotinin, 5 μg/mL leupeptin). Cell debris was removed by centrifugation (12000×g, 15 min, 4° C.) and the lysate was precleared by two successive incubations with 50 μL of a 1:1 slurry of protein A sepharose for 1 hour each. One-half mL of the cleared supernatant was reacted with 10 μL of protein A purified kinase-specific antisera (generated from the GST fusion protein or antipeptide antisera) plus 50 μL of a 1:1 slurry of protein A-sepharose for 2 hr at 4° C. The beads were then washed 2 times in PBSTDS, and 2 times in HNTG (20 mM HEPES, pH 7.5/150 mM NaCl, 0.1% Triton X-100, 10% glycerol).

The immunopurified kinases on sepharose beads are resuspended in 20 μL HNTG plus 30 mM MgCl₂, 10 mM MnCl₂, and 20 μCi [α³²P]ATP (3000 Ci/mmol). The kinase reactions are run for 30 min at room temperature, and stopped by addition of HNTG supplemented with 50 mM EDTA. The samples are washed 6 times in HNTG, boiled 5 min in SDS sample buffer and analyzed by 6% SDS-PAGE followed by autoradiography. Phosphoamino acid analysis is performed by standard 2D methods on ³²P-labeled bands excised from the SDS-PAGE gel.

Similar assays are performed on bacterially expressed GST-fusion constructs of the kinases.

### Example 8a

### Chromosomal Localization of Protein Kinases

Materials and Methods

Several sources were used to find information about the chromosomal localization of each of the genes described in this patent. First, cytogenetic map locations of these contigs were found in the title or text of their Genbank record, or by inspection through the NCBI human genome map viewer (http://www.ncbi.nlm.nih.gov/cgi-bin/Entrez/hum_srch?). Alternatively, the accession number for the nucleic acid sequence was used to query the Unigene database. The site containing the Unigene search engine is: http://www.ncbi.nlm.nih.gov/UniGene/Hs.Home.html. Information on map position within the Unigene database is imported from several sources, including the Online Mendelian Inheritance in Man (OMIM, http://www.ncbi.nlm.nih.gov/Omim/searchomim.html), The Genome Database (http://gdb.infobiogen.fr/gdb/simpleSearch.html), and the Whitehead Institute human physical map (http://carbon.wi.mit.edu:8000/cgi-bin/contig/sts_info?database=release).

Once a cytogenetic region has been identified by one of these approaches, disease association is established by searching OMIM with the cytogenetic location. OMIM maintains a searchable catalog of cytogenetic map locations organized by disease. A thorough search of available literature for the cytogenetic region is also made using Medline (http://www.ncbi.nlm.nih.gov/PubMed/medline.html). References for association of the mapped sites with chromosomal abnormalities found in human cancer can be found in: Knuutila, et al., Am J Pathol, 1998, 152:1107-1123.

Results

The chromosomal regions for mapped genes are listed below. The chromosomal positions were cross-checked with the Online Mendelian Inheritance in Man database (OMIM, http://www.ncbi.nlm.nih.gov/htbin-post/Omim)., which tracks genetic information for many human diseases, including cancer. References for association of the mapped sites with chromosomal abnormalities found in human cancer can be found in: Knuutila, et al., Am J Pathol, 1998, 152:1107-1123. A third source of information on mapped positions was searching published literature (at NCBI, http://www.ncbi.nlm.nih.gov/entrez/query.fcgi) for documented association of the mapped position with human disease.

SGK187, CRIK (SEQ ID NO:1, ENCODING SEQ ID NO:58 maps to chromosomal position 12q24.23. Amplification of this chromosomal position has been associated with non-small cell lung cancer (12q24.1-24.3; 2/50) (Knuutila, et al.).

SGK064, GRK7 (SEQ ID NO:2, ENCODING SEQ ID NO:59 maps to chromosomal position 3q24. This chromosomal position has been associated with squamous cell carcinomas of the head and neck (3/30) and Uterine cervix cancer (3/10). (Knuutila, et al.); Usher syndrome (OMIM, 276902 USHER SYNDROME, TYPE III; USH3)

SGK409, KIAA0303 (SEQ ID NO:3, ENCODING SEQ ID NO:60 maps to chromosomal position 5q12.1. This chromosomal position has been associated with cancer of the testis (15q15-qter; 2/11) (Knuutila, et al.).

SGK021 (SEQ ID NO:4, ENCODING SEQ ID NO:61 maps to chromosomal position 5q31.2. This chromosomal position has been associated with chondrosarcoma (2/45), (Knuutila, et al.).

SGK410 (SEQ ID NO:5, ENCODING SEQ ID NO:62 maps to chromosomal position Xq23. This chromosomal position has been associated with cancer of the prostate (Xq23-qter; 1/9), (Knuutila, et al.), and X-linked mental retardation (OMIM, 300046 MENTAL RETARDATION, X-LINKED NONSPECIFIC, 23; MRX23).

SGK069 (SEQ ID NO:6, ENCODING SEQ ID NO:63 maps to chromosomal position 19p11-p13. This chromosomal position has been associated with small cell lung cancer (19p12, 2/22), (Knuutila, et al.).

SGK110 (SEQ ID NO:7, ENCODING SEQ ID NO:64 maps to chromosomal position 19q13.4. This chromosomal position has been associated with the following human diseases: cancer of the breast (19q13.1-qter; 1/33), (Knuutila, et al.).

SGK053, CKLiK (SEQ ID NO:8, ENCODING SEQ ID NO:65 maps to chromosomal position 10p14. This chromosomal position has been associated with familial arrhythmogenic right ventricular dysplasia (OMIM, 604401 ARRHYTHMOGENIC RIGHT VENTRICULAR DYSPLASIA, FAMILIAL).

SGK124 (SEQ ID NO:9, ENCODING SEQ ID NO:66 maps to chromosomal position 20p12.2-p13. This chromosomal position has been associated with the following human diseases: cancer of the gastroesophageal junction (20p12; 3/28), (Knuutila, et al.), familial noncompaction of left ventricle (OMIM, 604169 NONCOMPACTION OF LEFT VENTRICULAR MYOCARDIUM, FAMILIAL ISOLATED, AUTOSOMAL DOMINANT).

SGK254, CAMKKa (SEQ ID NO:10, ENCODING SEQ ID NO:67 maps to chromosomal position 17p13.3. This chromosomal position has been associated with the following human diseases: Lost in cervical cancer (loss of heterogeneity, [Lazo, The molecular genetics of cervical carcinoma. Br J Cancer. August 1999;80(12):2008-18. Review].

SGK297, CaMKIb2 (SEQ ID NO:11, ENCODING SEQ ID NO:68 maps to chromosomal position Xq28. Translocations involving this chromosomal position has been associated with the following human diseases: T cell prolymphocytic leukemia (Laine, et al. Mol Cell. August 2000;6(2):395-407); mental retardation (e.g., Russo, et al., Am J Med Genet. Oct. 23, 2000;94(5):376-82). Also in Mantle cell lymphoma (Xq26-q28, 5/50).(Knuutila, et al.).

SGK411, CaMKII delta2 (SEQ ID NO:12, ENCODING SEQ ID NO:69 maps to chromosomal position 4q25. This chromosomal position has been associated with the following human diseases: developmental glaucoma (Rieger syndrome, iris hypoplasia, and iridogoniodysgenesis; Craig, et al. Curr Opin Ophthalmol. April 1999;10(2):126-34)

SGK027 (SEQ ID NO:13, ENCODING SEQ ID NO:70 maps to chromosomal position 5q11-q11.1. This chromosomal position has been associated with the following human diseases: malignant fibrous histiocytoma of bone (1/26) (Knuutila, et al.). Also with B-cell non-Hodgkin's lymphoma. (Wlodarska, et al. Cytogenet Cell Genet. 1994;65(3):179-83).

SGK046b (SEQ ID NO:14, ENCODING SEQ ID NO:71 maps to chromosomal position 3p24.1. This chromosomal position has been associated with the following human diseases: malignant fibrous histiocytoma of soft tissue (3p24-p26, 2/30) (Knuutila, et al.).

SGK046c (SEQ ID NO:15, ENCODING SEQ ID NO:72 maps to chromosomal position 3p24.1. This chromosomal position has been associated with the following human diseases: Malignant fibrous histiocytoma of soft tissue (3p24-p26, 2/30) (Knuutila, et al.).

SGK089 (SEQ ID NO:16, ENCODING SEQ ID NO:73 maps to chromosomal position 3p25.3. This chromosomal position has not been associated with human diseases.

SGK133 (SEQ ID NO:17, ENCODING SEQ ID NO:74 maps to chromosomal position 7p11.2-p21. This chromosomal position has been associated with the following human diseases: Malignant fibrous histiocytoma of bone (1/26), (Knuutila, et al.).

SGK004, MSK (SEQ ID NO:18, ENCODING SEQ ID NO:75 maps to chromosomal position 21q22.3. A gene which causes severe ocular alterations and occipital encephalocele (Knobloch syndrome) is mapped to 21q223 (Sertie, et al, Hum Mol Genet June 1996;5(6):843-7)

SGK006 (SEQ ID NO:19, ENCODING SEQ ID NO:76 maps to chromosomal position 16q16.1. This chromosomal position has not been associated with human diseases.

SGK180, SNRK (SEQ ID NO:20, ENCODING SEQ ID NO:77 maps to chromosomal position 3p21.31. This chromosomal position has been associated with the following human diseases: cancer of the bladder (1/14), (Knuutila, et al.).

SGK386, MLCKs (SEQ ID NO:21, ENCODING SEQ ID NO:78 maps to chromosomal position 20q11.1. This chromosomal position has been associated with the following human diseases: papillary renal cell carcinoma. (Lab Invest. March 1999;79(3):311-6).

SGK003 (SEQ ID NO:22, ENCODING SEQ ID NO:79 maps to chromosomal position 13q14.11. This chromosomal position has been associated with the following human diseases: fallopian tube cancer (13q14-qter; 1/12) (Knuutila, et al.).

SGK066 (SEQ ID NO:23, ENCODING SEQ ID NO:80 maps to chromosomal position 15q15. This chromosomal position has been associated with the following human diseases: cancer of the testis (2/11), (Knuutila, et al.).

SGK041, NKIAMRE (SEQ ID NO:24, ENCODING SEQ ID NO:81 maps to chromosomal position 5q31.1. This chromosomal position has been associated with the following human diseases: cancer of the digestive tract (5q31-qter), (Knuutila, et al.).

SGK112 (SEQ ID NO:25, ENCODING SEQ ID NO:82 maps to chromosomal position CHR2.

SGK038, ERK7 (SEQ ID NO:26, ENCODING SEQ ID NO:83 maps to chromosomal position na.

SGK158 (SEQ ID NO:27, ENCODING SEQ ID NO:84 maps to chromosomal position 1q42.11-42.2. This chromosomal position has not been associated with human diseases.

SGK429 (SEQ ID NO:28, ENCODING SEQ ID NO:85 maps to chromosomal position 7q34-35. This chromosomal position has been associated with the following human diseases: deafness (Mustapha, et al. Eur J Hum Genet. May-June 1998;6(3):245-50).

SGK152, SUDD (SEQ ID NO:29, ENCODING SEQ ID NO:86 maps to chromosomal position 18p11.1. This chromosomal position has not been associated with human diseases.

SGK077 (SEQ ID NO:30, ENCODING SEQ ID NO:87 maps to chromosomal position 17p13.3. This chromosomal position has been associated with the following human diseases: Lost in cervical cancer (loss of heterogeneity, Lazo, The molecular genetics of cervical carcinoma. Br J Cancer. August 1999;80(12):2008-18. Review).

SGK093, Wnk3 (SEQ ID NO:31, ENCODING SEQ ID NO:88 maps to chromosomal position 17q21.1-2. This chromosomal position has been associated with the following human diseases: cancer of the ovary (17q21-qter, 3/47), (Knuutila, et al.).

SGK074 (SEQ ID NO:32, ENCODING SEQ ID NO:89 maps to chromosomal position 19p 12-19q13. This chromosomal position has been associated with the following human diseases: Small cell lung cancer (2/22), (Knuutila, et al.).

SGK087 (SEQ ID NO:33, ENCODING SEQ ID NO:90 maps to chromosomal position 12p13.3. This chromosomal position has been associated with the following human diseases: hypertension (Disse-Nicodeme, et al. Am J Hum Genet. August 2000;67(2):302-10).

SGK295, KIS (SEQ ID NO:34, ENCODING SEQ ID NO:91 maps to chromosomal position 1q23.3. This chromosomal position has been associated with the following human diseases: Hematologic neoplasms (11q23-qter; 1/1), (Knuutila, et al.).

SGK419 (SEQ ID NO:35, ENCODING SEQ ID NO:92 maps to chromosomal position 4q24. This chromosomal position has been associated with the following human diseases: Malignant fibrous histiocytoma of soft tissue (14q24-q31; 1/58), (Knuutila, et al.).

SGK125, MYO3A (SEQ ID NO:36, ENCODING SEQ ID NO:93 maps to chromosomal position 10p12.32. This chromosomal position has been associated with the following human diseases: Mantle cell lymphoma (10p12-p13; 2/45), (Knuutila, et al.).

SGK445 (SEQ ID NO:37, ENCODING SEQ ID NO:94 maps to chromosomal position na. (Knuutila, et al.).

SGK127 (SEQ ID NO:38, ENCODING SEQ ID NO:95 maps to chromosomal position 12q24.21. This chromosomal position has been associated with the following human diseases: cancer of the respiratory tract and of the female genital organs (12q24.2). (Knuutila, et al.).

SGK009, ANKRD3 (SEQ ID NO:39, ENCODING SEQ ID NO:96 maps to chromosomal position 21q22.3. A gene which causes severe ocular alterations and occipital encephalocele (Knobloch syndrome) is mapped to 21q223 (Sertie, et al, Hum Mol Genet June 1996;5(6):843-7) (Knuutila, et al.).

SGK421, STK22A, TSK1 (SEQ ID NO:40, ENCODING SEQ ID NO:97 maps to chromosomal position 5q31.1. This chromosomal position has been associated with the following human diseases: Chondrosarcoma (5q31-q32; 2/45), (Knuutila, et al.).

SGK047 (SEQ ID NO:41, ENCODING SEQ ID NO:98 maps to chromosomal position 10p11.21. This chromosomal position has been associated with the following human diseases: Squamous cell carcinomas of the head and neck (10 p11-p13; 1/30), (Knuutila, et al.).

SGK196 (SEQ ID NO:42, ENCODING SEQ ID NO:99 maps to chromosomal position na.

SGK396 (SEQ ID NO:43, ENCODING SEQ ID NO:100 maps to chromosomal position na.

SGK279, PKN (SEQ ID NO:44, ENCODING SEQ ID NO:101 maps to chromosomal position 16q22.3. This chromosomal position has been associated with the following human diseases: Diffuse large cell lymphoma of stomach (16q22-ter; 1/7), (Knuutila, et al.).

SGK037 (SEQ ID NO:45, ENCODING SEQ ID NO:102 maps to chromosomal position 13q14.12. This chromosomal position has been associated with the following human diseases: fallopian tube cancer (13q14-qter; 1/12) (Knuutila, et al.).

SGK060 (SEQ ID NO:46, ENCODING SEQ ID NO:103 maps to chromosomal position 3q22.1. This chromosomal position has been associated with the following human diseases: Mantle cell lymphoma (13q22-q32; 3/72), (Knuutila, et al.).

SGK080 (SEQ ID NO:47, ENCODING SEQ ID NO:104 maps to chromosomal position 22q11.2. This chromosomal position has been associated with the following human diseases: Non-small cell lung cancer (1/50), (Knuutila, et al.).

SGK002 (SEQ ID NO:48, ENCODING SEQ ID NO:105 maps to chromosomal position 7q32.2. This chromosomal position has been associated with the following human diseases: Non-small cell lung cancer 7q32-q35; 1/50), (Knuutila, et al.).

SGK058 (SEQ ID NO:49, ENCODING SEQ ID NO:106 maps to chromosomal position 2q21.2. This chromosomal position has been associated with the following human diseases: bladder carcinoma (12q21-q24; 1/16), (Knuutila, et al.).

SGK103 (SEQ ID NO:50, ENCODING SEQ ID NO:107 maps to chromosomal position 5p14.3. This chromosomal position has been associated with the following human diseases: Malignant fibrous histiocytoma of soft tissue (6/88), (Knuutila, et al.).

SGK035 (SEQ ID NO:51, ENCODING SEQ ID NO:108 maps to chromosomal position CHR15.

SGK075 (SEQ ID NO:52, ENCODING SEQ ID NO:109 maps to chromosomal position 2q31.1. This chromosomal position has been associated with the following human diseases: Squamous cell carcinomas of the head and neck (2q31-q33; 3/30), (Knuutila, et al.).

SGK188, EphA9 (SEQ ID NO:53, ENCODING SEQ ID NO:110 maps to chromosomal position 1p34.1-34.3. This chromosomal position has been associated with the following human diseases: cancer of the testis (1p34-pter; 1/11), (Knuutila, et al.).

SGK040 (SEQ ID NO:54, ENCODING SEQ ID NO:111 maps to chromosomal position 12q12. This chromosomal position has been associated with the following human diseases: Diffuse large cell lymphoma (2/66), (Knuutila, et al.).

SGK390 (SEQ ID NO:55, ENCODING SEQ ID NO:112 maps to chromosomal position 13q14.2. This chromosomal position has been associated with the following human diseases: fallopian tube cancer (13q14-qter; 1/12) (Knuutila, et al.).

SGK007 (SEQ ID NO:56, ENCODING SEQ ID NO:113 maps to chromosomal position 10q26.11. This chromosomal position has not been associated with human diseases. (Knuutila, et al.).

SGK050 (SEQ ID NO:57, ENCODING SEQ ID NO:114 maps to chromosomal position 9p13.1-p13.2. This chromosomal position has been associated with the following human diseases: Non-small cell lung cancer (1/50) and testicular cancer (4/11) (Knuutila, et al.).

### Example 8b

### Candidate Single Nucleotide Polymorphisms (SNPs)

Materials and Methods

The most common variations in human DNA are single nucleotide polymorphisms (SNPs), which occur approximately once every 100 to 300 bases. Because SNPs are expected to facilitate large-scale association genetics studies, there has recently been great interest in SNP discovery and detection. Candidate SNPs for the genes in this patent were identified by blastn searching the nucleic acid sequences against the public database of sequences containing documented SNPs (dbSNP: sequence files were downloaded from ftp://ncbi.nlm.nih.gov/SNP/human/rs-fasta/ and ftp://ncbi.nlm.nih.gov/SNP/human/ss-fasta/ and used to create a blast database). dbSNP accession numbers for the SNP-containing sequences are given. SNPs were also identified by comparing several databases of expressed genes (dbEST, NRNA) and genomic sequence (i.e., NRNA) for single basepair mismatches. The results are shown in Table 2, in the column labeled “SNPs”. These are candidate SNPs—their actual frequency in the human population was not determined. The code below is standard for representing DNA sequence:
G=Guanosine
A=Adenosine
T=Thymidine
C=Cytidine
R=G or A, puRine
Y═C or T, pYrimidine
K=G or T, Keto
W=A or T, Weak (2 H-bonds)
S═C or G, Strong (3 H-bonds)
M=A or C, aMino
B═C, G or T (i.e., not A)
D=A, G or T (i.e., notC)
H=A, C or T (i.e., notG)
V=A, C or G (i.e., not T)
N=A, C, G or T, aNy

X=A, C, G or T

| | |
|---|---|
| complementary | G A T C R Y W S K M B V D H N X |
| DNA | +−+−+−+−+−+−+−+−+−+−+−+−+−+−+−+−+ |
| strands | C T A G Y R S W M K V B H D N X |

For example, if two versions of a gene exist, one with a “C” at a given position, and a second one with a “T: at the same position, then that position is represented as a Y, which means C or T. In table 2, for SGK002, the SNP column says “1165=R”, which means that at position 1165, a polymorphism exists, with that position sometimes containing a G and sometimes an A (R represents A or G). SNPs may be important in identifying heritable traits associated with a gene.
Results

SGK187, CRIK (SEQ ID NO:1, ENCODING SEQ ID NO:58 contains candidate single nucleotide polymorphisms at the following positions: 2874=R (ss1337340); 2883=Y (rs904655); 3327=R (ss 1581624). The sequences preceding the SNPs are: 3327=R (SEQ ID NO: 203) (gggttcgagagctgcagagR) dbSNP ss1581624; 2883=Y (SEQ ID NO: 204) (agagaccacagcagaagcY) dbSNP rs904655; 2874=R (SEQ ID NO: 205) (gctggaagagaccacR) dbSNP ss1337340).

SGK064, GRK7 (SEQ ID NO:2, ENCODING SEQ ID NO:59 contains candidate single nucleotide polymorphisms at the following positions: 965=K; 1318=R. The sequences preceding the SNPs are: 965=K (SEQ ID NO: 206) (catgaagcctgagaatgK); 1318=R (SEQ ID NO: 207) (agaaaccagagcaacgcttaR).

SGK409, KIAA0303 (SEQ ID NO:3, ENCODING SEQ ID NO:60 contains candidate single nucleotide polymorphisms at the following positions: 6282=M ; 6327=M. The sequences preceding the SNPs are: 6327=M (SEQ ID NO: 208) (ccctccaaagactaagcaccccgaM); 6282=M (SEQ ID NO: 209) (cacagtgaaagcagcagccacaagccM).

SGK021 (SEQ ID NO:4, ENCODING SEQ ID NO:61 contains candidate single nucleotide polymorphisms at the following positions: 9=S; 97=R. The sequences preceding the SNPs are: 9=S (atgggagcS); 97=R (SEQ ID NO: 210) (agccattgggaaaggcR).

SGK410 (SEQ ID NO:5, ENCODING SEQ ID NO:62 contains candidate single nucleotide polymorphisms at the following positions: none detected.

SGK069 (SEQ ID NO:6, ENCODING SEQ ID NO:63 contains candidate single nucleotide polymorphisms at the following positions: 1180=S (ss1317629); 210=Y (ss1688813). The sequences preceding the SNPs are: 1180=S (SEQ ID NO: 211) (ctggttcggcctggccS) dbSNP ss1317629; 210=Y (SEQ ID NO: 212) (tgcaggacggggaY) dbSNP ss1688813.

SGK110 (SEQ ID NO:7, ENCODING SEQ ID NO:64 contains candidate single nucleotide polymorphisms at the following positions: 597=R (rs654439); 252=Y (ss661406). The sequences preceding the SNPs are: 597=R (SEQ ID NO: 213) (aacgtgctggtcttcgacccR) dbSNP rs654439; 252=Y (SEQ ID NO: 214) (ccgcgtgctcctY) dbSNP ss661406.

SGK053, CKLiK (SEQ ID NO:8, ENCODING SEQ ID NO:65 contains candidate single nucleotide polymorphisms at the following positions: 605=Y; 509=M. The sequences preceding the SNPs are: 605=Y (SEQ ID NO: 215) (aaccttacagcaaagccgY); 509=M (SEQ ID NO: 216) (atcagtgactttggattgtcaaM).

SGK124 (SEQ ID NO:9, ENCODING SEQ ID NO:66 contains candidate single nucleotide polymorphisms at the following positions: 188=S; 333=Y. The sequences preceding the SNPs are: 188=S (SEQ ID NO: 217) (tccagatcgtgcaactgctgtggS); 333=Y (SEQ ID NO: 218) (tggccgtgctggagccctaY).

SGK254, CAMKKa (SEQ ID NO:10, ENCODING SEQ ID NO:67 contains candidate single nucleotide polymorphisms at the following positions: 555=R (ss84265); 1148=R. The sequences preceding the SNPs are: 555=R (SEQ ID NO: 219) (gtcgccctcccccR) dbSNP ss84265; 1148=R (SEQ ID NO: 220) (tggtgtttcctgaggR).

SGK297, CaMKIb2 (SEQ ID NO:11, ENCODING SEQ ID NO:68 contains candidate single nucleotide polymorphisms at the following positions: 77=K. The sequences preceding the SNPs are: 77=K (SEQ ID NO: 221) (ggctcggctcgggtgcctK).

SGK411, CaMKII delta2 (SEQ ID NO:12, ENCODING SEQ ID NO:69 contains candidate single nucleotide polymorphisms at the following positions: 15=M; 1387=S (ss1531091). The sequences preceding the SNPs are: 15=M; atggcttcgaccacM (SEQ ID NO: 222); 1387=S (SEQ ID NO: 223) (ccgggatggaaagtggS) dbSNP ss1531091.

SGK027 (SEQ ID NO:13, ENCODING SEQ ID NO:70 contains candidate single nucleotide polymorphisms at the following positions: 45=Y. The sequences preceding the SNPs are: 45=Y (SEQ ID NO: 224) (tgaatggaggtggcctggtgaacccccaY).

SGK046b (SEQ ID NO:14, ENCODING SEQ ID NO:71 contains candidate single nucleotide polymorphisms at the following positions: none detected.

SGK046c (SEQ ID NO:15, ENCODING SEQ ID NO:72 contains candidate single nucleotide polymorphisms at the following positions: none detected.

SGK089 (SEQ ID NO:16, ENCODING SEQ ID NO:73 contains candidate single nucleotide polymorphisms at the following positions: none detected.

SGK133 (SEQ ID NO:17, ENCODING SEQ ID NO:74 contains candidate single nucleotide polymorphisms at the following positions: 2003=S;1673=S. The sequences preceding the SNPs are: 2003=S (SEQ ID NO: 225) (gcttcagggccgagtacaaggccagtgS); 1673=S (SEQ ID NO: 226) (caccccccagccccggcgS).

SGK004, MSK (SEQ ID NO:18, ENCODING SEQ ID NO:75 contains candidate single nucleotide polymorphisms at the following positions: 1853=Y (ss571239). The sequences preceding the SNPs are: 1853=Y (SEQ ID NO: 227) (aggtgtgccaggccccY) dbSNP ss571239.

SGK006 (SEQ ID NO:19, ENCODING SEQ ID NO:76 contains candidate single nucleotide polymorphisms at the following positions: 4=S (ss1609852). The sequences preceding the SNPs are: 4=S (SEQ ID NO: 228) (Sctctgtatttcaggaagctct) dbSNP ss1609852.

SGK180, SNRK (SEQ ID NO:20, ENCODING SEQ ID NO:77 contains candidate single nucleotide polymorphisms at the following positions: 1817=S. The sequences preceding the SNPs are: 1817=S (SEQ ID NO: 229) (agccccagtgagaacaatgctggtggggS).

SGK386, MLCKs (SEQ ID NO:21, ENCODING SEQ ID NO:78 contains candidate single nucleotide polymorphisms at the following positions: 835=M. The sequences preceding the SNPs are: 835=M (SEQ ID NO: 230) (ttttggatgattgcccgccaM).

SGK003 (SEQ ID NO:22, ENCODING SEQ ID NO:79 contains candidate single nucleotide polymorphisms at the following positions: none detected.

SGK066 (SEQ ID NO:23, ENCODING SEQ ID NO:80 contains candidate single nucleotide polymorphisms at the following positions: none detected.

SGK041, NKIAMRE (SEQ ID NO:24, ENCODING SEQ ID NO:81 contains candidate single nucleotide polymorphisms at the following positions: 1033=R; 1284=R; 1181=Y. The sequences preceding the SNPs are: 1033=R (SEQ ID NO: 231) (ctaagtagttcagttttgggaR);1284=R (SEQ ID NO: 232) (tccacattgcggaggttctgtR); 1181=Y (SEQ ID NO: 233) (gcaaatgaaaatgttcatcctaY).

SGK112 (SEQ ID NO:25, ENCODING SEQ ID NO:82 contains candidate single nucleotide polymorphisms at the following positions: none detected.

SGK038, ERK7 (SEQ ID NO:26, ENCODING SEQ ID NO:83 contains candidate single nucleotide polymorphisms at the following positions: none detected.

SGK158 (SEQ ID NO:27, ENCODING SEQ ID NO:84 contains candidate single nucleotide polymorphisms at the following positions: 1752=Y (ss1529336). The sequences preceding the SNPs are: 1752=Y (SEQ ID NO: 234) (atcctggttctgtgcctgagggagctgttY) dbSNP ss1529336.

SGK429 (SEQ ID NO:28, ENCODING SEQ ID NO:85 contains candidate single nucleotide polymorphisms at the following positions: 196=R; 919=Y (ss1549835); 1865=Y (ss1517749). The sequences preceding the SNPs are: 196=R (SEQ ID NO: 235) (cccctgacgttctgR); 919=Y (SEQ ID NO: 236) (aggccaccaacctcatcy) dbSNP ss1549835; 1865=Y (SEQ ID NO: 237) (CCTCCTCACGGGCCY) dbSNP ss1517749.

SGK152, SUDD (SEQ ID NO:29, ENCODING SEQ ID NO:86 contains candidate single nucleotide polymorphisms at the following positions: 972=M. The sequences preceding the SNPs are: 972=M (SEQ ID NO: 238) (AAACTAAATCCACGTAAGATCM).

SGK077 (SEQ ID NO:30, ENCODING SEQ ID NO:87 contains candidate single nucleotide polymorphisms at the following positions: 390=Y (ss1658885); 611=R (ss1629760); 985=Y (ss1629759). The sequences preceding the SNPs are: 390=Y (SEQ ID NO: 239) (agcacaccctgy) dbSNP ss1658885; 611=R (SEQ ID NO: 240) (cagccgtcccgagcgR) dbSNP ss1629760; 985=Y (SEQ ID NO: 241) (ttgtgccaaggggaay) dbSNP ss1629759.

SGK093, Wnk3 (SEQ ID NO:31, ENCODING SEQ ID NO:88 contains candidate single nucleotide polymorphisms at the following positions: 3279=K; 4078=M. The sequences preceding the SNPs are: 3279=K (SEQ ID NO: 242) (gcccccaacccctgagccaK); 4078=M (SEQ ID NO: 243) (caaaataatcagcaacattM).

SGK074 (SEQ ID NO:32, ENCODING SEQ ID NO:89 contains candidate single nucleotide polymorphisms at the following positions: none detected.

SGK087 (SEQ ID NO:33, ENCODING SEQ ID NO:90 contains candidate single nucleotide polymorphisms at the following positions: 269=R (ss88136). The sequences preceding the SNPs are: 269=R (SEQ ID NO: 244) (acttcacgagcagtcaggcR) dbSNP ss88136.

SGK295, KIS (SEQ ID NO:34, ENCODING SEQ ID NO:91 contains candidate single nucleotide polymorphisms at the following positions: 355=W. The sequences preceding the SNPs are: 355=W (SEQ ID NO: 245) (ctggatgtcagtgttW).

SGK419 (SEQ ID NO:35, ENCODING SEQ ID NO:92 contains candidate single nucleotide polymorphisms at the following positions: none

SGK125, MYO3A (SEQ ID NO:36, ENCODING SEQ ID NO:93 contains candidate single nucleotide polymorphisms at the following positions: 3145=Y; 3204=Y. The sequences preceding the SNPs are: 3145=Y (SEQ ID NO: 246) (attatcacgtggagcagttaaatY); 3204=Y (SEQ ID NO: 247) (gattcaagcttgtgtcagagcattY).

SGK445 (SEQ ID NO:37, ENCODING SEQ ID NO:94 contains candidate single nucleotide polymorphisms at the following positions: none detected.

SGK127 (SEQ ID NO:38, ENCODING SEQ ID NO:95 contains candidate single nucleotide polymorphisms at the following positions: 501=S (ss2005786). The sequences preceding the SNPs are: 501=S (SEQ ID NO: 248) (tccagtggcccacS) dbSNP ss2005786.

SGK009, ANKRD3 (SEQ ID NO:39, ENCODING SEQ ID NO:96 contains candidate single nucleotide polymorphisms at the following positions: none detected.

SGK421, STK22A, TSK1 (SEQ ID NO:40, ENCODING SEQ ID NO:97 contains candidate single nucleotide polymorphisms at the following positions: 510=M (ss2055126); 279=R (ss2055125). The sequences preceding the SNPs are: 510=M (SEQ ID NO: 249) (ACAGTGGTCGAATGGCM) dbSNP ss2055126; 279=R (SEQ ID NO: 250) (gtcatggagctcgcR) dbSNP ss2055125.

SGK047 (SEQ ID NO:41, ENCODING SEQ ID NO:98 contains candidate single nucleotide polymorphisms at the following positions: none detected.

SGK196 (SEQ ID NO:42, ENCODING SEQ ID NO:99 contains candidate single nucleotide polymorphisms at the following positions: 99=R. The sequences preceding the SNPs are: 99=R (SEQ ID NO: 251) (ctgctgatcatggccctgatgaatactctR).

SGK396 (SEQ ID NO:43, ENCODING SEQ ID NO:100 contains candidate single nucleotide polymorphisms at the following positions: none detected.

SGK279, PKN (SEQ ID NO:44, ENCODING SEQ ID NO:101 contains candidate single nucleotide polymorphisms at the following positions: 665=Y. The sequences preceding the SNPs are: 665=Y (SEQ ID NO: 252) (CCTGAGGTGTGCCAGGY).

SGK037 (SEQ ID NO:45, ENCODING SEQ ID NO:102 contains candidate single nucleotide polymorphisms at the following positions: none detected.

SGK060 (SEQ ID NO:46, ENCODING SEQ ID NO:103 contains candidate single nucleotide polymorphisms at the following positions: 1463=W. The sequences preceding the SNPs are: 1463=W (SEQ ID NO: 253) (tgatgcatttgattcctattgtgW).

SGK080 (SEQ ID NO:47, ENCODING SEQ ID NO:104 contains candidate single nucleotide polymorphisms at the following positions: 1159=R (ss1367671); 422=R (ss1855009). The sequences preceding the SNPs are: 1159=R (SEQ ID NO: 254) (aagttcatttcagtggR) dbSNP ss1367671; 422=R (SEQ ID NO: 255) (tactgtagtgcgtcgggR) dbSNP ss1855009.

SGK002 (SEQ ID NO:48, ENCODING SEQ ID NO:105 contains candidate single nucleotide polymorphisms at the following positions: 1165=R; 983=M. The sequences preceding the SNPs are: 1165=R (SEQ ID NO: 256) (tggaggccatctttggccR); 983=M, 1753=R. (SEQ ID NO: 257) (GGGGTGAGCGGCCAGCTCATM); 443=K, (SEQ ID NO: 258) (ACCTGGTGGACCTGCAGAAK); 1245=Y (SEQ ID NO: 259) (GCGCCCCAACAGCGGTY); 1261=K (SEQ ID NO: 260) (GGGATGGACAGCCK), 1753=R (SEQ ID NO: 261) (tggtccctgccggcgtgcctctR).

SGK058 (SEQ ID NO:49, ENCODING SEQ ID NO:106 contains candidate single nucleotide polymorphisms at the following positions: none detected.

SGK103 (SEQ ID NO:50, ENCODING SEQ ID NO:107 contains candidate single nucleotide polymorphisms at the following positions: none detected.

SGK035 (SEQ ID NO:51, ENCODING SEQ ID NO:108 contains candidate single nucleotide polymorphisms at the following positions: 2273=Y. The sequences preceding the SNPs are: 273=Y (SEQ ID NO: 262) (cagttttcactgctacY).

SGK075 (SEQ ID NO:52, ENCODING SEQ ID NO:109 contains candidate single nucleotide polymorphisms at the following positions: 889=R. The sequences preceding the SNPs are: 889=R (SEQ ID NO: 263) (tttctgcaaaaacagctggccR).

SGK188, EphA9 (SEQ ID NO:53, ENCODING SEQ ID NO:110 contains candidate single nucleotide polymorphisms at the following positions: 2104=Y (ss1986120); 2319=R. The sequences preceding the SNPs are: 2319=R (SEQ ID NO: 264) (gtggctgggcaactgatggR); 2104=Y (SEQ ID NO: 265) (gtagccgtgcaY) dbSNP ss1986120.

SGK040 (SEQ ID NO:54, ENCODING SEQ ID NO:111 contains candidate single nucleotide polymorphisms at the following positions: 1869=R, 1004=Y. The sequences preceding the SNPs are: 1869=R (SEQ ID NO: 266) (ggttgtgccccatggcctatggttgaR); 1004=Y (SEQ ID NO: 267) (gacttgattttggctgaccY).

SGK390 (SEQ ID NO:55, ENCODING SEQ ID NO:112 contains candidate single nucleotide polymorphisms at the following positions: 1314=R. The sequences preceding the SNPs are: 1314=R (SEQ ID NO: 268) (gggaggttcatatgacgatgacacccaR).

SGK007 (SEQ ID NO:56, ENCODING SEQ ID NO:113 contains candidate single nucleotide polymorphisms at the following positions: none detected.

SGK050 (SEQ ID NO:57, ENCODING SEQ ID NO:114 contains candidate single nucleotide polymorphisms at the following positions: none detected.

### Example 9

### Demonstration Of Gene Amplification By Southern Blotting

Materials and Methods

Nylon membranes are purchased from Boehringer Mannheim. Denaturing solution contains 0.4 M NaOH and 0.6 M NaCl. Neutralization solution contains 0.5 M Tris-HCL, pH 7.5 and 1.5 M NaCl. Hybridization solution contains 50% formamide, 6×SSPE, 2.5× Denhardt's solution, 0.2 mg/mL denatured salmon DNA, 0.1 mg/mL yeast tRNA, and 0.2% sodium dodecyl sulfate. Restriction enzymes are purchased from Boehringer Mannheim. Radiolabeled probes are prepared using the Prime-it II kit by Stratagene. The beta actin DNA fragment used for a probe template is purchased from Clontech.

Genomic DNA is isolated from a variety of tumor cell lines (such as MCF-7, MDA-MB-231, Calu-6, A549, HCT-15, HT-29, Colo 205, LS-180, DLD-1, HCT-116, PC3, CAPAN-2, MIA-PaCa-2, PANC-1, AsPc-1, BxPC-3, OVCAR-3, SKOV3, SW 626 and PA-1, and from two normal cell lines.

A 10 μg aliquot of each genomic DNA sample is digested with EcoR I restriction enzyme and a separate 10 μg sample is digested with Hind III restriction enzyme. The restriction-digested DNA samples are loaded onto a 0.7% agarose gel and, following electrophoretic separation, the DNA is capillary-transferred to a nylon membrane by standard methods (Sambrook, J. et al (1989) Molecular Cloning: A Laboratory Manual, Cold Spring Harbor Laboratory).

### Example 10

### Detection Of Protein-Protein Interaction Through Phage Display

Materials And Methods

Phage display provides a method for isolating molecular interactions based on affinity for a desired bait. cDNA fragments cloned as fusions to phage coat proteins are displayed on the surface of the phage. Phage(s) interacting with a bait are enriched by affinity purification and the insert DNA from individual clones is analyzed.

T7 Phage Display Libraries

All libraries were constructed in the T7Select1-1b vector (Novagen) according to the manufacturer's directions.

Bait Presentation

Protein domains to be used as baits are generated as C-terminal fusions to GST and expressed in E. coli. Peptides are chemically synthesized and biotinylated at the N-terminus using a long chain spacer biotin reagent.

Selection

Aliquots of refreshed libraries (10¹⁰-10¹² pfu) supplemented with PanMix and a cocktail of E. coli inhibitors (Sigma P-8465) are incubated for 1-2 hrs at room temperature with the immobilized baits. Unbound phage is extensively washed (at least 4 times) with wash buffer.

After 3-4 rounds of selection, bound phage is eluted in 100 μL of 1% SDS and plated on agarose plates to obtain single plaques.

Identification of Insert DNAs

Individual plaques are picked into 25 μL of 10 mM EDTA and the phage is disrupted by heating at 70° C. for 10 min. 2 μL of the disrupted phage are added to 50 μL PCR reaction mix. The insert DNA is amplified by 35 rounds of thermal cycling (94° C, 50 sec; 50° C, 1 min; 72° C., 1 min).

Composition of Buffer

10× PanMix

5% Triton X-100

10% non-fat dry milk (Carnation)

10 mM EGTA

250 mM NaF

250 μg/mL Heparin (sigma)

250 μg/mL sheared, boiled salmon sperm DNA (sigma)

0.05% Na azide

Prepared in PBS

Wash Buffer

PBS supplemented with:

0.5% NP-40
25 μl g/mL heparin
PCR reaction mix

| | | |
|---|---|---|
| 1.0 mL | 10× PCR buffer (Perkin-Elmer, with 15 mM Mg) | |
| 0.2 mL | each dNTPs (10 mM stock) | |
| 0.1 mL | T7UP primer (15 pmol/μL) | |
| | GGAGCTGTCGTATTCCAGTC | (SEQ ID NO: 269) |
| 0.1 mL | T7DN primer (15 pmol/μL) | |
| | AACCCCTCAAGACCCGTTTAG | (SEQ ID NO: 270) |
| 0.2 mL | 25 mM MgCl₂ or MgSO₄ to compensate for EDTA | |

Q.S. to 10 mL with distilled water
Add 1 unit of Taq polymerase per 50 μL reaction

LIBRARY: T7 Select1-H441

### Example 11

### FLK-1

An ELISA assay was conducted to measure the kinase activity of the FLK-1 receptor and more specifically, the inhibition or activation of TK activity on the FLK-1 receptor. Specifically, the following assay was conducted to measure kinase activity of the FLK-1 receptor in cells genetically engineered to express Flk-1.

Materials and Reagents

The following reagents and supplies were used:

1. Corning 96-well ELISA plates (Corning Catalog No. 25805-96);

2. Cappel goat anti-rabbit IgG (catalog no.55641);

3. PBS (Gibco Catalog No. 450-1300EB);

4. TBSW Buffer (50 mM Tris (pH 7.2), 150 mM NaCl and 0.1% Tween-20);

5. Ethanolamine stock (10% ethanolamine (pH 7.0), stored at 4° C.);

6. HNTG buffer (20 mM HEPES buffer (pH 7.5), 150 mM NaCl, 0.2% Triton X-100, and 10% glycerol);

7. EDTA (0.5 M (pH 7.0) as a 100× stock);

8. Sodium orthovanadate (0.5 M as a 100× stock);

9. Sodium pyrophosphate (0.2 M as a 100× stock);

10. NUNC 96 well V bottom polypropylene plates (Applied Scientific Catalog No. AS-72092);

11. NIH3T3 C7#3 Cells (FLK-1 expressing cells);

12. DMEM with 1× high glucose L-Glutamine (catalog No. 11965-050);

13. FBS, Gibco (catalog no. 16000-028);

14. L-glutamine, Gibco (catalog no. 25030-016);

15. VEGF, PeproTech, Inc. (catalog no. 100-20) (kept as 1 μg/100 μl stock in Milli-Q dH₂O and stored at −20° C.);

16. Affinity purified anti-FLK-1 antiserum;

17. UB40 monoclonal antibody specific for phosphotyrosine (see, Fendley, et al., 1990, Cancer Research 50:1550-1558);

18. EIA grade Goat anti-mouse IgG-POD (BioRad catalog no. 172-1011);

19. 2,2-azino-bis(3-ethylbenz-thiazoline-6-sulfonic acid (ABTS) solution (100 mM citric acid (anhydrous), 250 mM Na₂HPO₄ (pH 4.0), 0.5 mg/ml ABTS (Sigma catalog no. A-1888)), solution should be stored in dark at 4° C. until ready for use;

20. H₂O₂ (30% solution) (Fisher catalog no. H325);

21. ABTS/H₂O₂ (15 ml ABTS solution, 2 μl H₂O₂) prepared 5 minutes before use and left at room temperature;

22. 0.2 M HCl stock in H₂O;

23. dimethylsulfoxide (100%) (Sigma Catalog No. D-8418); and

24. Trypsin-EDTA (Gibco BRL Catalog No. 25200-049).

Protocol

The following protocol was used for conducting the assay:

1. Coat Corning 96-well ELISA plates with 1.0 μg per well Cappel Anti-rabbit IgG antibody in 0.1 M Na₂CO₃ pH 9.6. Bring final volume to 150 μl per well. Coat plates overnight at 4° C. Plates can be kept up to two weeks when stored at 4° C.

2. Grow cells in Growth media (DMEM, supplemented with 2.0 mM L-Glutamine, 10% FBS) in suitable culture dishes until confluent at 37° C., 5% CO₂.

3. Harvest cells by trypsinization and seed in Corning 25850 polystyrene 96-well round bottom cell plates, 25.000 cells/well in 200 μl of growth media.

4. Grow cells at least one day at 37° C., 5% CO₂.

5. Wash cells with D-PBS 1×.

6. Add 200 μl/well of starvation media (DMEM, 2.0 mM 1-Glutamine, 0.1% FBS). Incubate overnight at 37° C., 5% CO₂.

7. Dilute Compounds 1:20 in polypropylene 96 well plates using starvation media. Dilute dimethylsulfoxide 1:20 for use in control wells.

8. Remove starvation media from 96 well cell culture plates and add 162 μl of fresh starvation media to each well.

9. Add 18 μl of 1:20 diluted Compound dilution (from step 7) to each well plus the 1:20 dimethylsulfoxide dilution to the control wells (±VEGF), for a final dilution of 1:200 after cell stimulation. Final dimethylsulfoxide is 0.5%. Incubate the plate at 37° C., 5% CO₂ for two hours.

10. Remove unbound antibody from ELISA plates by inverting plate to remove liquid. Wash 3 times with TBSW+0.5% ethanolamine, pH 7.0. Pat the plate on a paper towel to remove excess liquid and bubbles.

11. Block plates with TBSW+0.5% Ethanolamine, pH 7.0, 150 μl per well. Incubate plate thirty minutes while shaking on a microtiter plate shaker.

12. Wash plate 3 times as described in step 10.

13. Add 0.5 μg/well affinity purified anti-FLU-1 polyclonal rabbit antiserum. Bring final volume to 150 μl/well with TBSW+0.5% ethanolamine pH 7.0. Incubate plate for thirty minutes while shaking.

14. Add 180 μl starvation medium to the cells and stimulate cells with 20 μl/well 10.0 mM sodium ortho vanadate and 500 ng/ml VEGF (resulting in a final concentration of 1.0 mM sodium ortho vanadate and 50 ng/ml VEGF per well) for eight minutes at 37° C., 5% CO₂. Negative control wells receive only starvation medium.

15. After eight minutes, media should be removed from the cells and washed one time with 200 μl/well PBS.

16. Lyse cells in 150 μl/well HNTG while shaking at room temperature for five minutes. HNTG formulation includes sodium ortho vanadate, sodium pyrophosphate and EDTA.

17. Wash ELISA plate three times as described in step 10.

18. Transfer cell lysates from the cell plate to ELISA plate and incubate while shaking for two hours. To transfer cell lysate pipette up and down while scrapping the wells.

19. Wash plate three times as described in step 10.

20. Incubate ELISA plate with 0.02 μg/well UB40 in TBSW+05% ethanolamine. Bring final volume to 150 μl/well. Incubate while shaking for 30 minutes.

21. Wash plate three times as described in step 10.

22. Incubate ELISA plate with 1:10,000 diluted EIA grade goat anti-mouse IgG conjugated horseradish peroxidase in TBSW+0.5% ethanolamine, pH 7.0. Bring final volume to 150 μl/well. Incubate while shaking for thirty minutes.

23. Wash plate as described in step 10.

24. Add 100 μl of ABTS/H₂O₂ solution to well. Incubate ten minutes while shaking.

25. Add 100 μl of 0.2 M HCl for 0.1 M HCl final to stop the color development reaction. Shake 1 minute at room temperature. Remove bubbles with slow stream of air and read the ELISA plate in an ELISA plate reader at 410 nm.

### Example 12

### HER-2 ELISA

Assay 1: EGF Receptor-HER2 Chimeric Receptor Assay In Whole Cells.

HER2 kinase activity in whole EGFR-NIH3T3 cells was measured as described below:

Materials and Reagents

The following materials and reagents were used to conduct the assay:

1. EGF: stock concentration: 16.5 ILM; EGF 201, TOYOBO, Co., Ltd. Japan.

2. 05-101 (UBI) (a monoclonal antibody recognizing an EGFR extracellular domain).

3. Anti-phosphotyrosine antibody (anti-Ptyr) (polyclonal) (see, Fendley, et al., supra).

4. Detection antibody: Goat anti-rabbit lgG horse radish peroxidase conjugate, TAGO, Inc., Burlingame, Calif.

5. TBST buffer:

| Tris-HCl, pH 7.2 | 50 mM |
|---|---|
| NaCl | 150 mM |
| Triton X-100 | 0.1 |

6. HNTG 5× stock:

| HEPES | 0.1 M |
|---|---|
| NaCl | 0.75 M |
| Glycerol | 50% |
| Triton X-100 | 1.0% |

7. ABTS stock:

| | | |
|---|---|---|
| Citric Acid | 100 | mM |
| Na₂HPO₄ | 250 | mM |
| HCl, conc. | 0.5 | pM |
| ABTS* | 0.5 | mg/ml |

*(2,2′-azinobis(3-ethylbenzthiazolinesulfonic acid)). Keep solution in dark at 4° C. until use.

8. Stock reagents of:
EDTA 100 mM pH 7.0
Na₃VO₄ 0.5 M
Na₄ (P₂O₇) 0.2 M

Protocol

The following protocol was used:

A. Pre-Coat ELISA Plate

1. Coat ELISA plates (Corning, 96 well, Cat. #25805-96) with 05-101 antibody at 0.5 g per well in PBS, 100 μl final volume/well, and store overnight at 4° C. Coated plates are good for up to 10 days when stored at 4° C.

2. On day of use, remove coating buffer and replace with 100 μl blocking buffer (5% Carnation Instant Non-Fat Dry Milk in PBS). Incubate the plate, shaking, at room temperature (about 23° C. to 25° C.) for 30 minutes. Just prior to use, remove blocking buffer and wash plate 4 times with TBST buffer.

B. Seeding Cells

1. An NIH3T3 cell line overexpressing a chimeric receptor containing the EGFR extracellular domain and intracellular HER2 kinase domain can be used for this assay.

2. Choose dishes having 80-90% confluence for the experiment. Trypsinize cells and stop reaction by adding 10% fetal bovine serum. Suspend cells in DMEM medium (10% CS DMEM medium) and centrifuge once at 1500 rpm, at room temperature for 5 minutes.

3. Resuspend cells in seeding medium (DMEM, 0.5% bovine serum), and count the cells using trypan blue. Viability above 90% is acceptable. Seed cells in DMEM medium (0.5% bovine serum) at a density of 10,000 cells per well, 100 μl per well, in a 96 well microtiter plate. Incubate seeded cells in 5% CO₂ at 37° C. for about 40 hours.

C. Assay Procedures

1. Check seeded cells for contamination using an inverted microscope. Dilute drug stock (10 mg/ml in DMSO) 1:10 in DMEM medium, then transfer 5 μl to a TBST well for a final drug dilution of 1:200 and a final DMSO concentration of 1%. Control wells receive DMSO alone. Incubate in 5% CO₂ at 37° C. for two hours.

2. Prepare EGF ligand: dilute stock EGF in DMEM so that upon transfer of 10 μl dilute EGF (1:12 dilution), 100 nM final concentration is attained.

3. Prepare fresh HNTG* sufficient for 100 □l per well; and place on ice.

HNTG* (10 ml):

| | |
|---|---|
| HNTG stock | 2.0 ml |
| milli-Q H₂O | 7.3 ml |
| EDTA, 100 mM, pH 7.0 | 0.5 ml |
| Na₃VO₄, 0.5 M | 0.1 ml |
| Na₄ (P₂O₇), 0.2 M | 0.1 ml |

4. After 120 minutes incubation with drug, add prepared SGF ligand to cells, 10 μl per well, to a final concentration of 100 nM. Control wells receive DMEM alone. Incubate, shaking, at room temperature, for 5 minutes.

5. Remove drug, EGF, and DMEM. Wash cells twice with PBS. Transfer HNTG* to cells, 100 μl per well. Place on ice for 5 minutes. Meanwhile, remove blocking buffer from other ELISA plate and wash with TBST as described above.

6. With a pipette tip securely fitted to a micropipettor, scrape cells from plate and homogenize cell material by repeatedly aspirating and dispensing the HNTG* lysis buffer. Transfer lysate to a coated, blocked, and washed ELISA plate. Incubate shaking at room temperature for one hour.

7. Remove lysate and wash 4 times with TBST. Transfer freshly diluted anti-Ptyr antibody to ELISA plate at 100 μl per well. Incubate shaking at room temperature for 30 minutes in the presence of the anti-Ptyr antiserum (1:3000 dilution in TBST).

8. Remove the anti-Ptyr antibody and wash 4 times with TBST. Transfer the freshly diluted TAGO anti-rabbit IgG antibody to the ELISA plate at 100 μl per well. Incubate shaking at room temperature for 30 minutes (anti-rabbit IgG antibody: 1:3000 dilution in TBST).

9. Remove TAGO detection antibody and wash 4 times with TBST. Transfer freshly prepared ABTS/H₂O₂ solution to ELISA plate, 100 μl per well. Incubate shaking at room temperature for 20 minutes. (ABTS/H₂O₂ solution: 1.0 μl 30% H₂O₂ in 10 ml ABTS stock).

10. Stop reaction by adding 50 μl 5 N H₂SO₄ (optional), and determine O.D. at 4 10 nm.

11. The maximal phosphotyrosine signal is determined by subtracting the value of the negative controls from the positive controls. The percent inhibition of phosphotyrosine content for extract-containing wells is then calculated, after subtraction of the negative controls.

### Example 13

### PDGF-R ELISA

All cell culture media, glutamine, and fetal bovine serum were purchased from Gibco Life Technologies (Grand Island, N.Y.) unless otherwise specified. All cells were grown in a humid atmosphere of 90-95% air and 5-10% CO₂ at 37° C. All cell lines were routinely subcultured twice a week and were negative for mycoplasma as determined by the Mycotect method (Gibco).

For ELISA assays, cells (U124 2, obtained from Joseph Schlessinger, NYU) were grown to 80-90% confluency in growth medium (MEM with 10% FBS, NEAA, 1 mM NaPyr and 2 mM GLN) and seeded in 96-well tissue culture plates in 0.5% serum at 25,000 to 30,000 cells per well. After overnight incubation in 0.5% serum-containing medium, cells were changed to serum-free medium and treated with test compound for 2 hr in a 5% CO₂, 37° C. incubator. Cells were then stimulated with ligand for 5-10 minute followed by lysis with HNTG (20 mM Hepes, 150 mM NaCl, 10% glycerol, 5 mM EDTA, 5 mM Na₃VO₄, 0.2% Triton X-100, and 2 mM NaPyr). Cell lysates (0.5 mg/well in PBS) were transferred to ELISA plates previously coated with receptor-specific antibody and which had been blocked with 5% milk in TBST (50 mM Tris-HCl pH 7.2, 150 mM NaCl and 0.1% Triton X-100) at room temperature for 30 min. Lysates were incubated with shaking for 1 hour at room temperature. The plates were washed with TBST four times and then incubated with polyclonal anti-phosphotyrosine antibody at room temperature for 30 minutes. Excess anti-phosphotyrosine antibody was removed by rinsing the plate with TBST four times. Goat anti-rabbit IgG antibody was added to the ELISA plate for 30 min at room temperature followed by rinsing with TBST four more times. ABTS (100 mM citric acid, 250 mM Na₂HPO₄ and 0.5 mg/ml 2,2′-azino-bis(3-ethylbenzthiazoline-6-sulfonic acid)) plus H₂O₂ (1.2 ml 30% H₂O₂ to 10 ml ABTS) was added to the ELISA plates to start color development. Absorbance at 4 10 nm with a reference wavelength of 630 nm was recorded about 15 to 30 min after ABTS addition.

### Example 14

### IGF-I Receptor ELISA

The following protocol may be used to measure phosphotyrosine level on IGF-I receptor, which indicates IGF-I receptor tyrosine kinase activity.

Materials and Reagents

The following materials and reagents were used:

1. The cell line used in this assay is 3T3/IGF-1R, a cell line genetically engineered to overexpresses IGF-1 receptor.

2. NIH3T3/IGF-1R is grown in an incubator with 5% CO₂ at 37° C. The growth media is DMEM+10% FBS (heat inactivated)+2 mM L-glutamine.

3. Affinity purified anti-IGF-IR antibody 17-69.

4. D-PBS:

| | | |
|---|---|---|
| KH₂PO₄ | 0.20 | g/L |
| K₂HPO₄ | 2.16 | g/L |
| KCl | 0.20 | g/L |
| NaCl | 8.00 | g/L(pH 7.2) |

5. Blocking Buffer: TBST plus 5% Milk (Carnation Instant Non-Fat Dry Milk).

6. TBST buffer:

| | | |
|---|---|---|
| Tris-HCl | 50 | mM |
| NaCl | 150 | mM (pH 7.2/HCl 10 N) |

| | |
|---|---|
| Triton X-100 | 0.1% |

Stock solution of TBS (10×) is prepared, and Triton X-100 is added to the buffer during dilution.

7. HNTG buffer:

| | | |
|---|---|---|
| HEPES | 20 | mM |
| NaCl | 150 | mM (pH 7.2/HCl 1 N) |

| | |
|---|---|
| Glycerol | 10% |
| Triton X-100 | 0.2% |

Stock solution (5×) is prepared and kept at 4° C.

8. EDTA/HCl: 0.5 M pH 7.0 (NaOH) as 100× stock.

9. Na₃VO₄: 0.5 M as 100× stock and aliquots are kept in −80° C.

10. Na₄ P₂O₇: 0.2 M as 100× stock.

11. Insulin-like growth factor-1 from Promega (Cat#G5 111).

12. Rabbit polyclonal anti-phosphotyrosine antiserum.

13. Goat anti-rabbit IgG, POD conjugate (detection antibody), Tago (Cat. No. 4 520, Lot No. 1802): Tago, Inc., Burlingame, Calif.

14. ABTS (2,2′-azinobis(3-ethylbenzthiazolinesulfonic acid)) solution:

| | | |
|---|---|---|
| Citric acid | 100 | mM |
| Na₂HPO₄ | 250 | mM (pH 4.0/1 N HCl) |
| ABTS | 0.5 | mg/ml |

ABTS solution should be kept in dark and 4° C. The solution should be discarded when it turns green.

15. Hydrogen Peroxide: 30% solution is kept in the dark and at 4° C.

Protocol

All the following steps are conducted at room temperature unless it is specifically indicated. All ELISA plate washings are performed by rinsing the plate with tap water three times, followed by one TBST rinse. Pat plate dry with paper towels.

A. Cell Seeding:

1. The cells, grown in tissue culture dish (Corning 25020-100) to 80-90% confluence, are harvested with Trypsin-EDTA (0.25%, 0.5 ml/D-100, GIBCO).

2. Resuspend the cells in fresh DMEM+10% FBS+2 mM L-Glutamine, and transfer to 96-well tissue culture plate (Coming, 25806-96) at 20,000 cells/well (100 μl/well). Incubate for 1 day then replace medium to serum-free medium (90/1 μl ) and incubate in 5% CO₂ and 37° C. overnight.

B. ELISA Plate Coating and Blocking:

1. Coat the ELISA plate (Corning 25805-96) with Anti-IGF-IR Antibody at 0.5 μg/well in 100 μI PBS at least 2 hours.

2. Remove the coating solution, and replace with 100 μl Blocking Buffer, and shake for 30 minutes. Remove the blocking buffer and wash the plate just before adding lysate.

C. Assay Procedures:

1. The drugs are tested in serum-free condition.

2. Dilute drug stock (in 100% DMSO) 1:10 with DMBM in 96-well poly-propylene plate, and transfer 10 μl/well of this solution to the cells to achieve final drug dilution 1:100, and final DMSO concentration of 1.0%. Incubate the cells in 5% CO₂ at 37° C. for 2 hours.

3. Prepare fresh cell lysis buffer (HNTG*)

| | |
|---|---|
| HNTG | 2 ml |
| EDTA | 0.1 ml |
| Na₃VO₄ | 0.1 ml |
| Na₄ (P₂O₇) | 0.1 ml |
| H₂0 | 7.3 ml |

4. After drug incubation for two hours, transfer 10 μl/well of 200 nM IGF-1 Ligand in PBS to the cells (Final Conc.=20 nM), and incubate at 5% CO₂ at 37° C. for 10 minutes.

5. Remove media and add 100 μl/well HNTG* and shake for 10 minutes. Look at cells under microscope to see if they are adequately lysed.

6. Use a 12-channel pipette to scrape the cells from the plate, and homogenize the lysate by repeated aspiration and dispensing. Transfer all the lysate to the antibody coated ELISA plate, and shake for 1 hour.

7. Remove the lysate, wash the plate, transfer anti-pTyr (1:3,000 with TBST) 100 μl/well, and shake for 30 minutes.

8. Remove anti-pTyr, wash the plate, transfer TAGO (1:3,000 with TBST) 100 μl/well, and shake for 30 minutes.

9. Remove detection antibody, wash the plate, and transfer fresh ABTS/H₂O₂ (1.2 μl H₂O₂ to 10 ml ABTS) 100 μl/well to the plate to start color development.

10. Measure OD at 4 10 nm with a reference wavelength of 630 nm in Dynatec MR5000.

### Example 15

### EGF Receptor ELISA

EGF Receptor kinase activity in cells genetically engineered to express human EGF-R was measured as described below:

Materials and Reagents

The following materials and reagents were used:

1. EGF Ligand: stock concentration=16.5 μM; EGF 201, TOYOBO, Co., Ltd. Japan.

2. 05-101 (UBI) (a monoclonal antibody recognizing an EGFR extracellular domain).

3. Anti-phosphotyosine antibody (anti-Ptyr) (polyclonal).

4. Detection antibody: Goat anti-rabbit IgG horse radish peroxidase conjugate, TAGO, Inc., Burlingame, Calif.

5. TBST buffer:

| Tris-HCl, pH 7 | 50 mM |
|---|---|
| NaCl | 150 mM |
| Triton X-100 | 0.1 |

6. HNTG 5× stock:

| HEPES | 0.1 M |
|---|---|
| NaCl | 0.75 M |
| Glycerol | 50 |
| Triton X-100 | 1.0% |

7. ABTS stock:

| | | |
|---|---|---|
| Citric Acid | 100 | mM |
| Na₂HPO₄ | 250 | mM |
| HCl, conc. | 4.0 | pH |
| ABTS* | 0.5 | mg/ml |

Keep solution in dark at 4° C. until used.

8. Stock reagents of:

| | | |
|---|---|---|
| EDTA | 100 | mM pH 7.0 |
| Na₃VO₄ | 0.5 | M |
| Na₄(P₂0₇) | 0.2 | M |

Protocol

The following protocol was used:

A. Pre-coat ELISA Plate

1. Coat ELISA plates (Corning, 96 well, Cat. #25805-96) with 05-101 antibody at 0.5 μg per well in PBS, 150 μl final volume/well, and store overnight at 4° C. Coated plates are good for up to 10 days when stored at 4° C.

2. On day of use, remove coating buffer and replace with blocking buffer (5% Carnation Instant Non—Fat Dry Milk in PBS). Incubate the plate, shaking, at room temperature (about 23° C. to 25° C.) for 30 minutes. Just prior to use, remove blocking buffer and wash plate 4 times with TBST buffer.

B. Seeding Cells

1. NIH 3T3/C7 cell line (Honegger, et al., 1987, Cell 51:199-209) can be use for this assay.

2. Choose dishes having 80-90% confluence for the experiment. Trypsinize cells and stop reaction by adding 10% CS DMEM medium. Suspend cells in DMEM medium (10% CS DMEM medium) and centrifuge once at 1000 rpm at room temperature for 5 minutes.

3. Resuspend cells in seeding medium (DMEM, 0.5% bovine serum), and count the cells using trypan blue. Viability above 90% is acceptable. Seed cells in DMEM medium (0.5% bovine serum) at a density of 10,000 cells per well, 100 μl per well, in a 96 well microtiter plate. Incubate seeded cells in 5% CO₂ at 37° C. for about 40 hours.

C. Assay Procedures.

1. Check seeded cells for contamination using an inverted microscope. Dilute drug stock (10 mg/ml in DMSO) 1:10 in DMEM medium, then transfer 5 μl to a test well for a final drug dilution of 1:200 and a final DMSO concentration of 1%. Control wells receive DMSO alone. Incubate in 5% CO₂ at 37° C. for one hour.

2. Prepare EGF ligand: dilute stock EGF in DMEM so that upon transfer of 10 μl dilute EGF (1:12 dilution), 25 nM final concentration is attained.

3. Prepare fresh 10 ml HNTG* sufficient for 100 μl per well wherein HNTG* comprises: HNTG stock (2.0 ml), milli-Q H₂O (7.3 ml), EDTA, 100 mM, pH 7.0 (0.5 ml), Na₃VO₄ 0.5 M (0.1 ml) and Na4(P2O₇), 0.2 M (0.1 ml).

4. Place on ice.

5. After two hours incubation with drug, add prepared EGF ligand to cells,10 μl per well, to yield a final concentration of 25 nM. Control wells receive DMEM alone. Incubate, shaking, at room temperature, for 5 minutes.

6. Remove drug, EGF, and DMEM. Wash cells twice with PBS. Transfer HNTG* to cells, 100 μl per well. Place on ice for 5 minutes. Meanwhile, remove blocking buffer from other ELISA plate and wash with TBST as described above.

7. With a pipette tip securely fitted to a micropipettor, scrape cells from plate and homogenize cell material by repeatedly aspirating and dispensing the HNTG* lysis buffer. Transfer lysate to a coated, blocked, and washed ELISA plate. Incubate shaking at room temperature for one hour.

8. Remove lysate and wash 4 times with TBST. Transfer freshly diluted anti-Ptyr antibody to ELISA plate at 100 μl per well. Incubate shaking at room temperature for 30 minutes in the presence of the anti-Ptyr antiserum (1:3000 dilution in TBST).

9. Remove the anti-Ptyr antibody and wash 4 times with TBST. Transfer the freshly diluted TAGO 30 anti-rabbit IgG antibody to the ELISA plate at 100 μl per well. Incubate shaking at room temperature for 30 minutes (anti-rabbit IgG antibody: 1:3000 dilution in TBST).

10. Remove detection antibody and wash 4 times with TBST. Transfer freshly prepared ABTS/H₂O₂ solution to ELISA plate, 100 μl per well. Incubate at room temperature for 20 minutes. ABTS/H₂O₂ solution: 1.2 μl 30% H₂O₂ in 10 ml ABTS stock.

11. Stop reaction by adding 50 μl 5 N H₂SO₄ (optional), and determine O.D. at 410 nm.

12. The maximal phosphotyrosine signal is determined by subtracting the value of the negative controls from the positive controls. The percent inhibition of phosphotyrosine content for extract-containing wells is then calculated, after subtraction of the negative controls.

### Example 16

### Met Autophosphorylation Assay—ELISA

This assay determines Met tyrosine kinase activity by analyzing Met protein tyrosine kinase levels on the Met receptor.

Materials and Reagents

The following materials and reagents were used:

1. HNTG (5× stock solution): Dissolve 23.83 g HEPES and 43.83 g NaCl in about 350 ml dH₂O. Adjust pH to 7.2 with HCl or NaOH, add 500 ml glycerol and 10 ml Triton X-100, mix, add dH₂O to 1 L total volume. To make 1 L of 1× working solution add 200 ml 5× stock solution to 800 ml dH₂O, check and adjust pH as necessary, store at 4° C.

2. PBS (Dulbecco's Phosphate-Buffered Saline), Gibco Cat. #450-1300EB (1× solution).

3. Blocking Buffer: in 500 ml dH₂O place 100 g BSA, 12.1 g Tris-pH7.5, 58.44 g NaCl and 10 ml Tween-20, dilute to 1 L total volume.

4. Kinase Buffer: To 500 ml dH₂O add 12.1 g TRIS pH7.2, 58.4 g NaCl, 40.7 g MgCl₂ and 1.9 g EGTA; bring to 1 L total volume with dH₂O.

5. PMSF (Phenylmethylsulfonyl fluoride), Sigma Cat. #P-7626, to 435.5 mg, add 100% ethanol to 25 ml total volume, vortex.

6. ATP (Bacterial Source), Sigma Cat. #A-7699, store powder at −20° C.; to make up solution for use, dissolve 3.31 mg in 1 ml dH₂O.

7. RC-20H HRPO Conjugated Anti-Phosphotyrosine, Transduction Laboratories Cat. #E120H.

8. Pierce 1-Step (TM) Turbo TMB-ELISA (3,3′, 5,5′-tetramethylbenzidine, Pierce Cat. #34022.

9. H₂SO₄, add 1 ml conc. (18 N) to 35 ml dH₂O.

10. Tris-HCl, Fischer Cat. #BP152-5; to 121.14 g of material, add 600 ml MilliQ H₂O, adjust pH to 7.5 (or 7.2) with HCl, bring volume to 1 L with MilliQ H₂O.

11. NaCl, Fischer Cat. #S271-10, make up 5 M solution.

12. Tween-20, Fischer Cat. #S337-500.

13. Na₃VO₄, Fischer Cat. #S454-50, to 1.8 g material add 80 ml MilliQ H₂O, adjust pH to 10.0 with HCl or NaOH, boil in microwave, cool, check pH, repeat procedure until pH stable at 10.0, add MilliQ H₂O to 100 ml total volume, make 1 ml aliquots and store at −80° C.

14. MgCl₂, Fischer Cat. #M33-500, make up 1 M solution.

15. HEPES, Fischer Cat. #BP310-500, to 200 ml MilliQ H₂O, add 59.6 g material, adjust pH to 7.5, bring volume to 250 ml total, sterile filter.

16. Albumin, Bovine (BSA), Sigma Cat. #A-4503, to 30 grams material add sterile distilled water to make total volume of 300 ml, store at 4° C.

17. TBST Buffer: to approx. 900 ml dH₂O in a 1 L graduated cylinder add 6.057 g TRIS and 8.766 g NaCl, when dissolved, adjust pH to 7.2 with HCl, add 1.0 ml Triton X-100 and bring to 1 L total volume with dH₂O.

18. Goat Affinity purified antibody Rabbit IgG (whole molecule), Cappel Cat. #55641.

19. Anti h-Met (C-28) rabbit polyclonal IgG antibody, Santa Cruz Chemical Cat. #SC-161.

20. Transiently Transfected EGFR/Met chimeric cells (EMR) (Komada, et al., 1993, Oncogene 8:2381-2390.

21. Sodium Carbonate Buffer, (Na₂CO₄, Fischer Cat. #S495): to 10.6 g material add 800 ml MilliQ H₂O, when dissolved adjust pH to 9.6 with NaOH, bring up to 1 L total volume with MilliQ H₂O, filter, store at 4° C.

Procedure

All of the following steps are conducted at room temperature unless it is specifically indicated otherwise. All ELISA plate washing is by rinsing 4× with TBST.

A. EMR Lysis

This procedure can be performed the night before or immediately prior to the start of receptor capture.

1. Quick thaw lysates in a 37° C. waterbath with a swirling motion until the last crystals disappear.

2. Lyse cell pellet with 1× HNTG containing 1 mM PMSF. Use 3 ml of HNTG per 15 cm dish of cells. Add ½ the calculated HNTG volume, vortex the tube for 1 min., add the remaining amount of HNTG, vortex for another min.

3. Balance tubes, centrifuge at 10,000×g for 10 min at 4° C.

4. Pool supernatants, remove an aliquot for protein determination.

5. Quick freeze pooled sample in dry ice/ethanol bath. This step is performed regardless of whether lysate will be stored overnight or used immediately following protein determination.

6. Perform protein determination using standard bicinchoninic acid (BCA) method (BCA Assay Reagent Kit from Pierce Chemical Cat. #23225).

B. ELISA Procedure

1. Coat Coming 96 well ELISA plates with 5 μg per well Goat anti-Rabbit antibody in Carbonate Buffer for a total well volume of 50 μl. Store overnight at 4° C.

2. Remove unbound Goat anti-rabbit antibody by inverting plate to remove liquid.

3. Add 150 μl of Blocking Buffer to each well. Incubate for 30 min. at room temperature with shaking.

4. Wash 4× with TBST. Pat plate on a paper towel to remove excess liquid and bubbles.

5. Add 1 μg per well of Rabbit anti-Met antibody diluted in TBST for a total well volume of 100 μl.

6. Dilute lysate in HNTG (90 μg lysate/100 μl)

7. Add 100 μl of diluted lysate to each well. Shake at room temperature for 60 min.

8. Wash 4× with TBST. Pat on paper towel to remove excess liquid and bubbles.

9. Add 50 μl of 1× lysate buffer per well.

10. Dilute compounds/extracts 1: 10 in 1× Kinase Buffer in a polypropylene 96 well plate.

11. Transfer 5.5 μl of diluted drug to ELISA plate wells. Incubate at room temperature with shaking for 20 min.

12. Add 5.5 μl of 60 μM ATP solution per well. Negative controls do not receive any ATP. Incubate at room temperature for 90 min., with shaking.

13. Wash 4× with TBST. Pat plate on paper towel to remove excess liquid and bubbles.

14. Add 100 μl per well of RC20 (1:3000 dilution in Blocking Buffer). Incubate 30 min. at room temperature with shaking.

15. Wash 4× with TBST. Pat plate on paper towel to remove excess liquid and bubbles.

16. Add 100 μl per well of Turbo-TMB. Incubate with shaking for 30-60 min.

17. Add 100 μl per well of 1 M H₂SO4 to stop reaction.

18. Read assay on Dynatech MR7000 ELISA reader. Test Filter=450 nm, reference filter=410 nm.

### Example 17

### Biochemical src Assay-ELISA

This assay is used to determine src protein kinase activity measuring phosphorylation of a biotinylated peptide as the readout.

Materials and Reagents

The following materials and reagents were used:

1. Yeast transformed with src.

2. Cell lysates: Yeast cells expressing src are pelleted, washed once with water, re-pelleted and stored at −80° C. until use.

3. N-terminus biotinylated (SEQ ID NO: 271) EEEYEEYEEEYEEEYEEEY is prepared by standard procedures well known to those skilled in the art.

4. DMSO: Sigma, St. Louis, Mo.

5. 96 Well ELISA Plate: Coming 96 Well Easy Wash, Modified flat Bottom Plate, Corning Cat. #25805-96.

6. NUNC 96-well V-bottom polypropylene plates for dilution of compounds: Applied Scientific Cat. #A-72092.

7. Vecastain ELITE ABC reagent: Vector, Burlingame, Calif.

8. Anti-src (327) mab: Schizosaccharomyces Pombe was used to express recombinant src (Superti-Furga, et al., EMBO J. 12:2625-2634; Superti-Furga, et al., Nature Biochem. 14:600-605). S. Pombe strain SP200 (h-s leul.32 ura4 ade210 ) was grown as described and transformations were pRSP expression plasmids were done by the lithium acetate method (Superti-Furga, supra). Cells were grown in the presence of 1 μM thiamin to repress expression from the nmtl promoter or in the absence of thiamin to induce expression.

9. Monoclonal anti-phosphotyrosine, UBI 05-321 (UB40 may be used instead).

10. Turbo TMB-ELISA peroxidase substrate: Pierce Chemical.

Buffer Solutions:

1. PBS (Dulbecco's Phosphate-Buffered Saline): GIBCO PBS, GIBCO Cat. #450-1300EB.

2. Blocking Buffer: 5% Non-fat milk (Carnation) in PBS.

3. Carbonate Buffer: Na₂CO₄ from Fischer, Cat. #S495, make up 100 mM stock solution.

4. Kinase Buffer: 1.0 ml (from 1 M stock solution) MgCl_{2; 0.2} ml (from a 1 M stock solution) MnCl_{2; 0.2} ml (from a 1 M stock solution) DTT; 5.0 ml (from a 1 M stock solution) HEPES; 0.1 ml TX-100; bring to 10 ml total volume with MilliQ H₂O.

5. Lysis Buffer: 5.0 HEPES (from 1 M stock solution.); 2.74 ml NaCl (from 5 M stock solution); 10 ml glycerol; 1.0 ml TX-100; 0.4 ml EDTA (from a 100 mM stock solution); 1.0 ml PMSF (from a 100 mM stock solution); 0.1 ml Na₃VO₄ (from a 0.1 M stock solution); bring to 100 ml total volume with MilliQ H₂O.

6. ATP: Sigma Cat. #A-7699, make up 10 mM stock solution (5.51 mg/ml).

7. TRIS-HCl: Fischer Cat. #BP 152-5, to 600 ml MilliQ H₂O add 121.14 g material, adjust pH to 7.5 with HCl, bring to 1 L total volume with MilliQ H₂O.

8. NaCl: Fischer Cat. #S271-10, Make up 5 M stock solution with MilliQ H₂O.

9. Na₃VO₄: Fischer Cat. #S454-50; to 80 ml MilliQ H₂O, add 1.8 g material; adjust pH to 10.0 with HCl or NaOH; boil in a microwave; cool; check pH, repeat pH adjustment until pH remains stable after heating/cooling cycle; bring to 100 ml total volume with MilliQ H₂O; make 1 ml aliquots and store at −80° C.

10. MgCl₂: Fischer Cat. #M33-500, make up 1 M stock solution with MilliQ H₂O.

11. HEPES: Fischer Cat. #BP 310-500; to 200 ml MilliQ H₂O, add 59.6 g material, adjust pH to 7.5, bring to 250 ml total volume with MilliQ H₂O, sterile filter (1 M stock solution).

12. TBST Buffer: TBST Buffer: To 900 ml dH₂O add 6.057 g TRIS and 8.766 g NaCl; adjust pH to 7.2 with HCl, add 1.0 ml Triton-X-100; bring to 1 L total volume with dH₂O.

13. MnCl₂: Fischer Cat. #M87-100, make up 1 M stock solution with MilliQ H₂O.

14. DTT; Fischer Cat. #BP172-5.

15. TBS (TRIS Buffered Saline): to 900 ml MilliQ H₂O add 6.057 g TRIS and 8.777 g NaCl; bring to 1 L total volume with MilliQ H₂O.

16. Kinase Reaction Mixture: Amount per assay plate (100 wells): 1.0 ml Kinase Buffer, 200 μg GST-ζ, bring to final volume of 8.0 ml with MilliQ H₂O.

17. Biotin labeled (SEQ ID NO: 271) EEEYEEYEEEYEEEYEEEY: Make peptide stock solution (1 mM, 2.98 mg/ml) in water fresh just before use.

18. Vectastain ELITE ABC reagent: To prepare 14 ml of working reagent, add 1 drop of reagent A to 15 ml TBST and invert tube several times to mix. Then add 1 drop of reagent B. Put tube on orbital shaker at room temperature and mix for 30 minutes.

Protocol

A. Preparation of src Coated ELISA Plate.

1. Coat ELISA plate with 0.5 μg/well anti-src mab in 100 μl of pH 9.6 sodium carbonate buffer at 4° C. overnight.

2. Wash wells once with PBS.

3. Block plate with 0.15 ml 5% milk in PBS for 30 min. at room temperature.

4. Wash plate 5× with PBS.

5. Add 10 μg/well of src transformed yeast lysates diluted in Lysis Buffer (0.1 ml total volume per well). (Amount of lysate may vary between batches.) Shake plate for 20 minutes at room temperature.

B. Preparation of Phosphotyrosine Antibody-Coated ELISA Plate.

1. 4G10 plate: coat 0.5 μg/well 4G10 in 100 μl PBS overnight at 4° C. and block with 150 μl of 5% milk in PBS for 30 minutes at room temperature.

C. Kinase Assay Procedure.

1. Remove unbound proteins from step 1-7, above, and wash plates 5× with PBS.

2. Add 0.08 ml Kinase Reaction Mixture per well (containing 10 μl of 10× Kinase Buffer and 10 μM (final concentration) biotin-EEEYEEYEEEYEEEYEEEY (SEQ ID NO: 271) per well diluted in water.

3. Add 10 μl of compound diluted in water containing 10% DMSO and pre-incubate for 15 minutes at room temperature.

4. Start kinase reaction by adding 10 μl/well of 0.05 mM ATP in water (5 μM ATP final).

5. Shake ELISA plate for 15 min. at room temperature.

6. Stop kinase reaction by adding 10 μl of 0.5 M EDTA per well.

7. Transfer 90 μl supernatant to a blocked 4G10 coated ELISA plate from section B, above.

8. Incubate for 30 min. while shaking at room temperature.

9. Wash plate 5× with TBST.

10. Incubate with Vectastain ELITE ABC reagent (100 μl/well) for 30 min. at room temperature.

11. Wash the wells 5× with TBST.

12. Develop with Turbo TMB.

### Example 18

### Biochemical lck Assay—ELISA

This assay is used to determine lck protein kinase activities measuring phosphorylation of GST-Q as the readout.

Materials and Reagents

The following materials and reagents were used:

1. Yeast transformed with lck. Schizosaccharomyces Pombe was used to express recombinant lck (Superti-Furga, et al., EMBO J. 12:2625-2634; Superti-Furga, et al., Nature Biotech. 14:600-605). S. Pombe strain SP200 (h-s leul.32 ura4 ade210) was grown as described and transformations with pRSP expression plasmids were done by the lithium acetate method (Superti-Furga, supra). Cells were grown in the presence of 1 μM thiamin to induce expression.

2. Cell lysates: Yeast cells expressing lck are pelleted, washed once in water, re-pelleted and stored frozen at −80° C. until use.

3. GST-ζ: DNA encoding for GST-ζ fusion protein for expression in bacteria obtained from Arthur Weiss of the Howard Hughes Medical Institute at the University of California, San Francisco. Transformed bacteria were grown overnight while shaking at 25° C. GST-ζ was purified by glutathione affinity chromatography, Pharmacia, Alameda, Calif.

4. DMSO: Sigma, St. Louis, Mo.

5. 96-Well ELISA plate: Corning 96 Well Easy Wash, Modified Flat Bottom Plate, Corning Cat. #25805-96.

6. NUNC 96-well V-bottom polypropylene plates for dilution of compounds: Applied Scientific Cat. #AS-72092.

7. Purified Rabbit anti-GST antiserum: Amrad Corporation (Australia) Cat. #90001605.

8. Goat anti-Rabbit-IgG-HRP: Amersham Cat. #V010301

9. Sheep ant-mouse IgG (H+L): Jackson Labs Cat. #5215-005-003.

10. Anti-lck (3A5) mab: Santa Cruz Biotechnology Cat #sc-433.

11. Monoclonal anti-phosphotyrosine UBI 05-321 (UB40 may be used instead).

Buffer Solutions:

1. PBS (Dulbecco's Phosphate-Buffered Saline) 1× solution: GIBCO PBS, GIBCO Cat. #450-1300EB.

2. Blocking Buffer: 100 g BSA, 12.1 g. TRIS-pH7.5, 58.44 g NaCl, 10 ml Tween-20, bring up to 1 L total volume with MilliQ H₂O.

3. Carbonate Buffer: Na₂CO₄ from Fischer, Cat. #S495; make up 100 mM solution with MilliQ H₂O.

4. Kinase Buffer: 1.0 ml (from 1 M stock solution) MgCl₂; 0.2 ml (from a 1 M stock solution) MnCl₂; 0.2 ml (from a 1 M stock solution) DTT; 5.0 ml (from a 1 M stock solution) HEPES; 0.1 ml TX-100; bring to 10 ml total volume with MilliQ H₂O.

5. Lysis Buffer: 5.0 HEPES (from 1 M stock solution.); 2.74 ml NaCl (from 5 M stock solution); 10 ml glycerol; 1.0 ml TX-100; 0.4 ml EDTA (from a 100 mM stock solution); 1.0 ml PMSF (from a 100 mM stock solution); 0.1 ml Na₃VO₄ (from a 0.1 M stock solution); bring to 100 ml total volume with MilliQ H₂O.

6. ATP: Sigma Cat. #A-7699, make up 10 mM stock solution (5.51 mg/ml).

7. TRIS-HCl: Fischer Cat. #BP 152-5, to 600 ml MilliQ H₂O add 121.14 g material, adjust pH to 7.5 with HCl, bring to 1 L total volume with MilliQ H₂O.

8. NaCl: Fischer Cat. #S271-10, Make up 5 M stock solution with MilliQ H₂O.

9. Na₃VO₄: Fischer Cat. #S454-50; to 80 ml MilliQ H₂O, add 1.8 g material; adjust pH to 10.0 with HCl or NaOH; boil in a microwave; cool; check pH, repeat pH adjustment until pH remains stable after heating/cooling cycle; bring to 100 ml total volume with MilliQ H₂O; make 1 ml aliquots and store at −80° C.

10. MgCl₂: Fischer Cat. #M33-500, make up 1 M stock solution with MilliQ H₂O.

11. HEPES: Fischer Cat. #BP 310-500; to 200 ml MilliQ H₂O, add 59.6 g material, adjust pH to 7.5, bring to 250 ml total volume with MilliQ H₂O, sterile filter (1M stock solution).

12. Albumin, Bovine (BSA), Sigma Cat. #A4503; to 150 ml MilliQ H₂O add 30 g material, bring 300 ml total volume with MilliQ H₂O, filter through 0.22 [Om filter, store at 4° C.

13. TBST Buffer: To 900 ml dH₂O add 6.057 g TRIS and 8.766 g NaCl; adjust pH to 7.2 with HCl, add 1.0 ml Triton-X-100; bring to 1 L total volume with dH₂O.

14. MnCl₂: Fischer Cat. #M87-100, make up 1 M stock solution with MilliQ H₂O.

15. DTT; Fischer Cat. #BP172-5.

16. TBS (TRIS Buffered Saline): to 900 ml MilliQ H₂O add 6.057 g TRIS and 8.777 g NaCl; bring to 1 L total volume with MilliQ H₂O.

17. Kinase Reaction Mixture: Amount per assay plate (100 wells): 1.0 ml Kinase Buffer, 200 μg GST-ζ, bring to final volume of 8.0 ml with MilliQ H₂O.

Procedures

A. Preparation of lck Coated ELISA Plate.

1. Coat 2.0 μg/well Sheep anti-mouse IgG in 100 μl of pH 9.6 sodium carbonate buffer at 4° C. overnight.

2. Wash well once with PBS.

3. Block plate with 0.15 ml of blocking Buffer for 30 min. at room temp.

4. Wash plate 5× with PBS.

5. Add 0.5 μg/well of anti-lck (mab 3A5) in 0.1 ml PBS at room temperature for 1-2 hours.

6. Wash plate 5× with PBS.

7. Add 20 μg/well of lck transformed yeast lysates diluted in Lysis Buffer (0.1 ml total volume per well). (Amount of lysate may vary between batches) Shake plate at 4° C. overnight to prevent loss of activity.

B. Preparation of Phosphotyrosine Antibody-Coated ELISA Plate.

1. UB40 plate: 1.0 μg/well UB40 in 100 μl of PBS overnight at 4° C. and block with 150 μl of Blocking Buffer for at least 1 hour.

C. Kinase Assay Procedure.

1. Remove unbound proteins from step 1-7, above, and wash plates 5× with PBS.

2. Add 0.08 ml Kinase Reaction Mixture per well (containing 10 μl of 10× Kinase Buffer and 2 μg GST-ζ per well diluted with water).

3. Add 10 μl of compound diluted in water containing 10% DMSO and pre-incubate for 15 minutes at room temperature.

4. Start kinase reaction by adding 10 μl/well of 0.1 mM ATP in water (10 μM ATP final).

5. Shake ELISA plate for 60 min. at room temperature.

6. Stop kinase reaction by adding 10 vl of 0.5 M EDTA per well.

7. Transfer 90 μl supernatant to a blocked 4G10 coated ELISA plate from section B, above.

8. Incubate while shaking for 30 min. at room temperature.

9. Wash plate 5× with TBST.

10. Incubate with Rabbit anti-GST antibody at 1:5000 dilution in 100 μl TBST for 30 min. at room temperature.

11. Wash the wells 5× with TBST.

12. Incubate with Goat anti-Rabbit-IgG-HRP at 1:20,000 dilution in 100 μl of TBST for 30 min. at room temperature.

13. Wash the wells 5× with TBST.

14. Develop with Turbo TMB.

### Example 19

Biochemical c-Kit Assay—ELISA

A. Materials And Reagents

1) HNTG: 5× stock concentration: 100 mM HEPES pH 7.2, 750 mM NaCl, 50% glycerol, 2.5% Triton X-100.

2) PBS (Dulbecco's Phosphate-Buffered Saline): Gibco Catalog #450-1300EB

3) 1× Blocking Buffer: 10 mM TRIS-pH7.5, 1% BSA, 100 mM NaCl, 0.1% Triton X-100

4) 1× Kinase Buffer: 25 mM HEPES, 100 mM NaCl, 10 mM Mg Cl₂, 6 mM Mn Cl₂.

5) PMSF Stock Solution=100 (Sigma Catalog #P-7626)

6) 10 mM ATP (Bacterial source) Sigma A-7699, 5g.

7) UB40 anti-phosphotyrosine mAb (available from Terrance at Sugen.

8) HRP conjugated sheep anti-Mouse IgG. (Amersham NA 931)

9) ABTS (5Prime-3Prime 7-579844)

10) TRIS HCL: Fisher BP 152-5

11) NaCl: Fisher S271-10

12) Triton X-100: Fisher BP151-100

13) Na₃VO₄: Fisher S454-50

14) MgCl₂: Fisher M33-500

15) MnCl₂: Fisher M87-500

16) HEPES: Fisher BP310-500

17) Albumin, Bovine (BSA): Sigma A-8551

18) TBST Buffer: 50 mM Tris pH 7.2, 150 mM NaCl, 0.1% Triton X-100.

19) Goat affinity purified antibody Rabbit IgG (whole molecule): Cappel 55641.

20) Anti Kit (C-20) rabbit polyclonal IgG antibody: Santa Cruz sc-168

21) Kit/CHO cells: CHO cells stably expressing GyrB/Kit, which are grown in standard CHO medium, supplemented with 1 mg/ml G418

22) Indolinone Compounds: The indolinone compounds were synthesized as set forth in the following application: PCT application number US99/06468, filed Mar. 26, 1999 by Fong, et al. and entitled METHODS OF MODULATING TYROSINE PROTEIN KINASE (Lyon & Lyon docket number 231/250 PCT which is hereby incorporated by reference in its entirety including any drawings.

B. Procedure

All of the following steps are conducted at room temperature unless it is specifically indicated. All ELISA plate washing is by rinsing 4× with TBST.

Kit Cell Lysis

This procedure is performed 1 hour prior to the start of receptor capture.

1) Wash a >95% confluent 15 cm dish with PBS and aspirate as much as possible.

2) Lyse the cells with 3 ml of 1× HNTG containing 1 mM PMSF/15 cm dish. Scrape the cells from the plate and transfer to a 50 ml centrifuge tube.

3) Pool supernatants, and allow to sit, on ice, for one hour with occasional vortexing. Failure to do so with result in an increased background (approximately 3-fold higher).

4) Balance tubes and centrifuge at 10,000×g for 10 min at 4[]C. Remove an aliquot for protein determination

5) Perform protein determination as per the SOP for protein determination using the bicinchoninic acid (BCA) method.

ELISA Procedure

1) Coat Corning 96-well ELISA plates with 2 μg per well Goat anti-rabbit antibody in PBS for a total well volume of 100 μl. Store overnight at 4° C.

2) Remove unbound Goat anti-rabbit antibody by inverting plate to remove liquid.

3) Add 100 μl of Blocking Buffer to each well. Shake at room temperature for 60 mm.

4) Wash 4× with TBST. Pat plate on a paper towel to remove excess liquid and bubbles

5) Add 0.2 μg per well of Rabbit anti-Kit antibody diluted in TBST for a total well volume of 100 μl. Shake at room temperature for 60 min.

6) Dilute lysate in HNTG (180 μg lysate/100 μl)

7) Add 100 μl of diluted lysate to each well. Shake at room temperature for 60 min.

8) Wash 4× with TBST. Pat plate on a paper towel to remove excess liquid and bubbles.

9) Dilute compounds/extracts (or as stated otherwise) in 1× kinase buffer, with 5μM ATP in a polypropylene 96 well plate.

10) Transfer 100 μl of diluted drug to ELISA plate wells. Incubate at room temperature with shaking for 60 min.

11) Stop reaction with the addition of 10 μl of 0.5 M EDTA. Plate is now stable for a reasonable period of time.

12) Wash 4× with TBST. Pat plate on a paper towel to remove excess liquid and bubbles.

13) Add 100 μl per well of UB40 (1:2000 dilution in TBST). Incubate 60 min at room temperature, with shaking.

14) Wash 4× with TBST. Pat plate on a paper towel to remove excess liquid and bubbles.

15) Add 100 μl per well of sheep anti-mouse IgG-HRP (1:5000 dilution in TBST). Incubate 60 min at room temperature, with shaking.

16) Wash 4× with TBST. Pat plate on a paper towel to remove excess liquid and bubbles.

17) Add 100 μl per well of ABTS. Incubate with shaking for 15-30 min.

18) Read assay on Dynatech MR7000 ELISA reader
Test Filter=410 nm
Reference Filter=630 nm.

### Example 20

### Assay Measuring Phosphorylating Function of RAF

The following assay reports the amount of RAF-catalyzed phosphorylation of its target protein MEK as well as MEK's target MAPK. The RAF gene sequence is described in Bonner et al., 1985, Molec. Cell. Biol. 5:1400-1407, and is readily accessible in multiple gene sequence data banks. Construction of the nucleic acid vector and cell lines utilized for this portion of the invention are fully described in Morrison et al., 1988, Proc. Natl. Acad. Sci. USA 85:8855-8859.

Materials and Reagents

1. Sf9 (Spodoptera frugiperda) cells; GIBCO-BRL, Gaithersburg, Md.

2. RIPA buffer: 20 mM Tris/HCl pH 7.4, 137 mM NaCl, 10% glycerol, 1 mM PMSF, 5 mg/L Aprotenin, 0.5% Triton X-100.

3. Thioredoxin-MEK fusion protein (T-MEK): T-MEK expression and purification by affinity chromatography were performed according to the manufacturer's procedures. Catalog#K 350-01 and R 350-40, Invitrogen Corp., San Diego, Calif.

4. His-MAPK (ERK 2); His-tagged MAPK was expressed in XL1 Blue cells transformed with pUC18 vector encoding His-MAPK. His-MAPK was purified by Ni-affinity chromatography. Cat#27-4949-01, Pharmacia, Alameda, Calif., as described herein.

5. Sheep anti mouse IgG: Jackson laboratories, West Grove, Pa. Catalog, #515-006-008, Lot#28563.

6. RAF-1 protein kinase specific antibody: URP2653 from UBI.

7. Coating buffer: PBS; phosphate buffered saline, GIBCO-BRL, Gaithersburg, Md.

8. Wash buffer: TBST-50 mM Tris/HCl pH 7.2, 150 mM NaCl, 0.1% Triton X-100.

9. Block buffer: TBST, 0.1% ethanolamine pH 7.4.

10. DMSO, Sigma, St. Louis, Mo.

11. Kinase buffer (KB): 20 mM HEPES/HCl pH 7.2, 150 mM NaCl, 0.1% Triton X-100, 1 mM PMSF, 5 mg/L Aprotenin, 75 mM sodium ortho vanadate, 0.5 MM DTT and 10 mM MgCl₂.

12. ATP mix: 100 mM MgCl₂, 300 mM ATP, 10 mCi 33P ATP (Dupont-NEN)/ml.

13. Stop solution: 1% phosphoric acid; Fisher, Pittsburgh, Pa.

14. Wallac Cellulose Phosphate Filter mats; Wallac, Turku, Finnland.

15. Filter wash solution: 1% phosphoric acid, Fisher, Pittsburgh, Pa.

16. Tomtec plate harvester, Wallac, Turku, Finnland.

17. Wallac beta plate reader #1205, Wallac, Turku, Finnland.

18. NUNC 96-well V bottom polypropylene plates for compounds Applied Scientific Catalog #AS-72092.

Protocol

All of the following steps were conducted at room temperature unless specifically indicated.

1. ELISA plate coating: ELISA wells are coated with 100 ml of Sheep anti mouse affinity purified antiserum (1 mg/100 ml coating buffer) over night at 4° C. ELISA plates can be used for two weeks when stored at 4° C.

2. Invert the plate and remove liquid. Add 100 ml of blocking solution and incubate for 30 min.

3. Remove blocking solution and wash four times with wash buffer. Pat the plate on a paper towel to remove excess liquid.

4. Add 1 mg of antibody specific for RAF-1 to each well and incubate for 1 hour. Wash as described in step 3.

5. Thaw lysates from RAS/RAF infected Sf9 cells and dilute with TBST to 10 mg/100 ml. Add 10 mg of diluted lysate to the wells and incubate for 1 hour. Shake the plate during incubation. Negative controls receive no lysate. Lysates from RAS/RAF infected Sf9 insect cells are prepared after cells are infected with recombinant baculoviruses at a MOI of 5 for each virus, and harvested 48 hours later. The cells are washed once with PBS and lysed in RIPA buffer. Insoluble material is removed by centrifugation (5 min at 10,000×g). Aliquots of lysates are frozen in dry ice/ethanol and stored at −80° C. until use.

6. Remove non-bound material and wash as outlined above (step 3).

7. Add 2 mg of T-MEK and 2 mg of His-MAEPK per well and adjust the volume to 40 ml with kinase buffer. Methods for purifying T-MEK and MAPK from cell extracts are provided herein by example.

8. Pre-dilute compounds (stock solution 10 mg/ml DMSO) or extracts 20 fold in TBST plus 1% DMSO. Add 5 ml of the pre-diluted compounds/extracts to the wells described in step 6. Incubate for 20 min. Controls receive no drug.

9. Start the kinase reaction by addition of 5 ml ATPmix; Shake the plates on an ELISA plate shaker during incubation.

10. Stop the kinase reaction after 60 min by addition of 30 ml stop solution to each well.

11. Place the phosphocellulose mat and the ELISA plate in the Tomtec plate harvester. Harvest and wash the filter with the filter wash solution according to the manufacturers recommendation. Dry the filter mats. Seal the filter mats and place them in the holder. Insert the holder into radioactive detection apparatus and quantify the radioactive phosphorous on the filter mats.

Alternatively, 40 ml aliquots from individual wells of the assay plate can be transferred to the corresponding positions on the phosphocellulose filter mat. After air drying the filters, put the filters in a tray. Gently rock the tray, changing the wash solution at 15 min intervals for 1 hour. Air-dry the filter mats. Seal the filter mats and place them in a holder suitable for measuring the radioactive phosphorous in the samples. Insert the holder into a detection device and quantify the radioactive phosphorous on the filter mats.

### Example 21

### CDK2/Cyclin A—Inhibition Assay

This assay analyzes the protein kinase activity of CDK2 in exogenous substrate.

Materials and Reagents

1. Buffer A (80 mM Tris (pH 7.2), 40 mM MgCl₂): 4.84 g Tris (F.W.=121.1 g/mol), 4.07 g MgCl₂ (F.W.=203.31 g/mol) dissolved in 500 ml H₂O. Adjust pH to 7.2 with HCl.

2. Histone H1 solution (0.45 mg/ml Histone H1 and 20 mM HEPES pH 7.2: 5 mg Histone H1 (Boehinger Mannheim) in 11.111 ml 20 mM HEPES pH 7.2 (477 mg HEPES (F.W.=238.3 g/mol) dissolved in 100 ml ddH₂O), stored in 1 ml aliquots at −80° C.

3. ATP solution (60 μM ATP, 300 μg/ml BSA, 3 mM DTT): 120 μl 10 mM ATP, 600 μl 10 mg/ml BSA to 20 ml, stored in 1 ml aliquots at −80° C.

4. CDK2 solution: cdk2/cyclin A in 10 mM HEPES pH 7.2, 25 mM NaCl, 0.5 mM DTT, 10% glycerol, stored in 9 μl aliquots at −80° C.

Description of Assay:

1. Prepare solutions of inhibitors at three times the desired final assay concentration in ddH₂O/15% DMSO by volume.

2. Dispense 20 μl of inhibitors to wells of polypropylene 96-well plates (or 20 μl 15% DMSO for positive and negative controls).

3. Thaw Histone H1 solution (1 ml/plate), ATP solution (1 ml/plate plus 1 aliquot for negative control), and CDK2 solution (9 μ/plate). Keep CDK2 on ice until use. Aliquot CDK2 solution appropriately to avoid repeated freeze-thaw cycles.

4. Dilute 9 μl CDK2 solution into 2.1 ml Buffer A (per plate). Mix. Dispense 20 μl into each well.

5. Mix 1 ml Histone H1 solution with 1 ml ATP solution (per plate) into a 10 ml screw cap tube. Add γ³³P ATP to a concentration of 0.15 μCi/20 μl (0.15 μCi/well in assay). Mix carefully to avoid BSA frothing. Add 20 μl to appropriate wells. Mix plates on plate shaker. For negative control, mix ATP solution with an equal amount of 20 mM HEPES pH 7.2 and add γ³³P ATP to a concentration of 0.15 μCi/20 μl solution. Add 20 μl to appropriate wells.

6. Let reactions proceed for 60 minutes.

7. Add 35 μl 10% TCA to each well. Mix plates on plate shaker.

8. Spot 40 μl of each sample onto P30 filter mat squares. Allow mats to dry (approx. 10-20 minutes).

9. Wash filter mats 4×10 minutes with 250 ml 1% phosphoric acid (10 ml phosphoric acid per liter ddH₂O).

10. Count filter mats with beta plate reader.

### CELLULAR/BIOLOGIC ASSAYS

### Example 22

### PDGF-Induced BrdU Incorporation Assay

Materials and Reagents:

1. PDGF: human PDGF B/B; 1276-956, Boehringer Mannheim, Germany

2. BrdU Labeling Reagent: 10 mM, in PBS (pH7.4), Cat. No. 1647 229, Boehringer Mannheim, Germany.

3. FixDenat: fixation solution (ready to use), Cat. No. 1647 229, Boehringer Mannheim, Germany.

4. Anti-BrdU-POD: mouse monoclonal antibody conjugated with peroxidase, Cat. No. 1647 229, Boehringer Mannheim, Germany.

5. TMB Substrate Solution: tetramethylbenzidine (TMB), ready to use, Cat. No. 1 647 229, Boehringer Mannheim, Germany.

6. PBS Washing Solution: 1× PBS, pH 7.4, made in house (Sugen, Inc., Redwood City, Calif.).

7. Albumin, Bovine (BSA): Fraction V powder; A-8551, Sigma Chemical Co., USA.

8. 3T3 cell line genetically engineered to express human PDGF-R.

Protocol:

1. Cells are seeded at 8000 cells/well in DMEM, 10% CS, 2 mM Gln in a 96 well plate. Cells are incubated overnight at 37° C. in 5% CO₂.

2. After 24 hours, the cells are washed with PBS, and then are serum starved in serum free medium (0% CS DMEM with 0.1% BSA) for 24 hours.

3. On day 3, ligand (PDGF, 3.8 nM, prepared in DMEM with 0.1% BSA) and test compounds are added to the cells simultaneously. The negative control wells receive serum free DMEM with 0.1% BSA only; the positive control cells receive the ligand (PDGF) but no test compound. Test compounds are prepared in serum free DMEM with ligand in a 96 well plate, and serially diluted for 7 test concentrations.

4. After 20 hours of ligand activation, diluted BrdU labeling reagent (1:100 in DMEM, 0.1%,BSA) is added and the cells are incubated with BrdU (final concentration=10 μM) for 1.5 hours.

5. After incubation with labeling reagent, the medium is removed by decanting and tapping the inverted plate on a paper towel. FixDenat solution is added (50 μl/well) and the plates are incubated at room temperature for 45 minutes on a plate shaker.

6. The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

7. The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

8. The antibody conjugate is thoroughly removed by decanting and rinsing the wells 5 times with PBS, and the plate is dried by inverting and tapping on a paper towel.

9. TMB substrate solution is added (100 μl/well) and incubated for 20 minutes at room temperature on a plate shaker until color development is sufficient for photometric detection.

10. The absorbence of the samples are measured at 410 nm (in “dual wavelength” mode with a filter reading at 490 nm, as a reference wavelength) on a Dynatech ELISA plate reader.

### Example 23

### EGF-Induced BrdU Incorporation Assay

Materials and Reagents

1. EGF: mouse EGF, 201; Toyobo, Co., Ltd. Japan

2. BrdU Labeling Reagent: 10 mM, in PBS (pH7.4), Cat. No. 1 647 229, Boehringer Mannheim, Germany.

3. FixDenat: fixation solution (ready to use), Cat. No. 1 647 229, Boehringer Mannheim, Germany.

4. Anti-BrdU-POD: mouse monoclonal antibody conjugated with peroxidase, Cat. No. 1 647 229, Boehringer Mannheim, Germany.

5. TMB Substrate Solution: tetramethylbenzidine (TMB), ready to use, Cat. No. 1 647 229, Boehringer Mannheim, Germany.

6. PBS Washing Solution: 1× PBS, pH 7.4.

7. Albumin, Bovine (BSA): Fraction V powder; A-855 1, Sigma Chemical Co., USA.

8. 3T3 cell line genetically engineered to express human EGF-R.

Protocol

1. Cells are seeded at 8000 cells/well in 10% CS, 2mM Gln in DMEM, in a 96 well plate. Cells are incubated overnight at 37° C. in 5% CO₂.

2. After 24 hours, the cells are washed with PBS, and then are serum starved in serum free medium (0% CS DMEM with 0.1% BSA) for 24 hours.

3. On day 3, ligand (EGF, 2 nM, prepared in DMEM with 0.1% BSA) and test compounds are added to the cells simultaneously. The negative control wells receive serum free DMEM with 0.1% BSA only; the positive control cells receive the ligand (EGF) but no test compound. Test compounds are prepared in serum free DMEM with ligand in a 96 well plate, and serially diluted for 7 test concentrations.

4. After 20 hours of ligand activation, diluted BrdU labeling reagent (1:100 in DMEM, 0.1% BSA) is added and the cells are incubated with BrdU (final concentration=10 μM) for 1.5 hours.

5. After incubation with labeling reagent, the medium is removed by decanting and tapping the inverted plate on a paper towel. FixDenat solution is added (50 μl/well) and the plates are incubated at room temperature for 45 minutes on a plate shaker.

6. The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

7. The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

8. The antibody conjugate is thoroughly removed by decanting and rinsing the wells 5 times with PBS, and the plate is dried by inverting and tapping on a paper towel.

9. TMB substrate solution is added (100 μl/well) and incubated for 20 minutes at room temperature on a plate shaker until color development is sufficient for photometric detection.

10. The absorbence of the samples are measured at 410 nm (in “dual wavelength” mode with a filter reading at 490 nm, as a reference wavelength) on a Dynatech ELISA plate reader.

### Example 24

### EGF-Induced HER2-Driven BrdU Incorporation

Materials and Reagents:

1. EGF: mouse EGF, 201; Toyobo, Co., Ltd. Japan

2. BrdU Labeling Reagent: 10 mM, in PBS (pH 7.4), Cat. No. 1 647 229, Boehringer Mannheim, Germany.

3. FixDenat: fixation solution (ready to use), Cat. No. 1 647 229, Boehringer Mannheim, Germany.

4. Anti-BrdU-POD: mouse monoclonal antibody conjugated with peroxidase, Cat. No. 1 647 229, Boehringer Mannheim, Germany.

5. TMB Substrate Solution: tetramethylbenzidine (TMB), ready to use, Cat. No. 1 647 229, Boehringer Mannheim, Germany.

6. PBS Washing Solution: 1× PBS, pH 7.4, made in house.

7. Albumin, Bovine (BSA): Fraction V powder; A-8551, Sigma Chemical Co., USA.

8. 3T3 cell line engineered to express a chimeric receptor having the extra-cellular domain of EGF-R and the intra-cellular domain of HER2.

Protocol:

1. Cells are seeded at 8000 cells/well in DMEM, 10% CS, 2 mM Gln in a 96-well plate. Cells are incubated overnight at 37° C. in 5% CO₂.

2. After 24 hours, the cells are washed with PBS, and then are serum starved in serum free medium (0% CS DMEM with 0.1% BSA) for 24 hours.

3. On day 3, ligand (EGF=2 nM, prepared in DMEM with 0.1% BSA) and test compounds are added to the cells simultaneously. The negative control wells receive serum free DMEM with 0.1% BSA only; the positive control cells receive the ligand (EGF) but no test compound. Test compounds are prepared in serum free DMEM with ligand in a 96 well plate, and serially diluted for 7 test concentrations.

4. After 20 hours of ligand activation, diluted BrdU labeling reagent (1:100 in DMEM, 0.1% BSA) is added and the cells are incubated with BrdU (final concentration=10 μM) for 1.5 hours.

5. After incubation with labeling reagent, the medium is removed by decanting and tapping the inverted plate on a paper towel. FixDenat solution is added (50 μl/well) and the plates are incubated at room temperature for 45 minutes on a plate shaker.

6. The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

7. The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

8. The antibody conjugate is thoroughly removed by decanting and rinsing the wells 5 times with PBS, and the plate is dried by inverting and tapping on a paper towel.

9. TMB substrate solution is added (100 μl/well) and incubated for 20 minutes at room temperature on a plate shaker until color development is sufficient for photometric detection.

10. The absorbence of the samples are measured at 410 nm (in “dual wavelength” mode with a filter reading at 490 nm, as a reference wavelength) on a Dynatech ELISA plate reader.

### Example 25

### IGF1-Induced BrdU Incorporation Assay

Materials and Reagents:

1. IGF I Ligand: human, recombinant; G511, Promega Corp, USA.

2. BrdU Labeling Reagent: 10 mM, in PBS (pH 7.4), Cat. No. 1 647 229, Boehringer Mannheim, Germany.

3. FixDenat: fixation solution (ready to use), Cat. No. 1 647 229, Boehringer Mannheim, Germany.

4. Anti-BrdU-POD: mouse monoclonal antibody conjugated with peroxidase, Cat. No. 1 647 229, Boehringer Mannheim, Germany.

5. TMB Substrate Solution: tetramethylbenzidine (TMB), ready to use, Cat. No. 1 647 229, Boehringer Mannheim, Germany.

6. PBS Washing Solution: 1× PBS, pH 7.4.

7. Albumin, Bovine (BSA): Fraction V powder; A-8551, Sigma Chemical Co., USA.

8. 3T3 cell line genetically engineered to express human IGF-1 receptor.

Protocol:

1. Cells are seeded at 8000 cells/well in DMEM, 10% CS, 2 mM Gln in a 96-well plate. Cells are incubated overnight at 37° C. in 5% CO₂.

2. After 24 hours, the cells are washed with PBS, and then are serum starved in serum free medium (0% CS DMEM with 0.1% BSA) for 24 hours.

3. On day 3, ligand (IGF1=3.3 nM, prepared in DMEM with 0.1% BSA) and test compounds are added to the cells simultaneously. The negative control wells receive serum free DMEM with 0.1% BSA only; the positive control cells receive the ligand (IGF1) but no test compound. Test compounds are prepared in serum free DMEM with ligand in a 96 well plate, and serially diluted for 7 test concentrations.

4. After 16 hours of ligand activation, diluted BrdU labeling reagent (1:100 in DMEM, 0.1% BSA) is added and the cells are incubated with BrdU (final concentration=10 μM) for 1.5 hours.

5. After incubation with labeling reagent, the medium is removed by decanting and tapping the inverted plate on a paper towel. FixDenat solution is added (50 μl/well) and the plates are incubated at room temperature for 45 minutes on a plate shaker.

6. The FixDenat solution is thoroughly removed by decanting and tapping the inverted plate on a paper towel. Milk is added (5% dehydrated milk in PBS, 200 μl/well) as a blocking solution and the plate is incubated for 30 minutes at room temperature on a plate shaker.

7. The blocking solution is removed by decanting and the wells are washed once with PBS. Anti-BrdU-POD solution (1:100 dilution in PBS, 1% BSA) is added (100 μl/well) and the plate is incubated for 90 minutes at room temperature on a plate shaker.

8. The antibody conjugate is thoroughly removed by decanting and rinsing the wells 5 times with PBS, and the plate is dried by inverting and tapping on a paper towel.

9. TMB substrate solution is added (100 μl/well) and incubated for 20 minutes at room temperature on a plate shaker until color development is sufficient for photometric detection.

10. The absorbence of the samples are measured at 410 nm (in “dual wavelength” mode with a filter reading at 490 nm, as a reference wavelength) on a Dynatech ELISA plate reader.

### Example 26

### HUV-EC-C Assay

The following protocol may also be used to measure a compound's activity against PDGF-R, FGF-R, VEGF, aFGF or Flk-I/KDR, all of which are naturally expressed by HUV-EC cells.

DAY 0

1. Wash and trypsinize HUV-EC-C cells (human umbilical vein endothelial cells, (American Type Culture Collection; catalogue no. 1730 CRL). Wash with Dulbecco's phosphate-buffered saline (D-PBS; obtained from Gibco BRL; catalogue no. 14190-029) 2 times at about 1 ml/0 cm² of tissue culture flask. Trypsinize with 0.05% trypsin-EDTA in non-enzymatic cell dissociation solution (Sigma Chemical Company; catalogue no. C-1544). The 0.05% trypsin was made by diluting 0.25% trypsin/1 mM EDTA (Gibco; catalogue no. 25200-049) in the cell dissociation solution. Trypsinize with about 1 ml/25-30 cm² of tissue culture flask for about 5 minutes at 37° C. After cells have detached from the flask, add an equal volume of assay medium and transfer to a 50 ml sterile centrifuge tube (Fisher Scientific; catalogue no. 05-539-6).

2. Wash the cells with about 35 ml assay medium in the 50 ml sterile centrifuge tube by adding the assay medium, centrifuge for 10 minutes at approximately 200 g, aspirate the supernatant, and resuspend with 35 ml D-PBS. Repeat the wash two more times with D-PBS, resuspend the cells in about 1 ml assay medium/15 cm² of tissue culture flask. Assay medium consists of F12K medium (Gibco BRL; catalogue no. 21127-014)+0.5% heat-inactivated fetal bovine serum. Count the cells with a Coulter Counter™ Coulter Electronics, Inc.) and add assay medium to the cells to obtain a concentration of 0.8−10×105 cells/ml.

3. Add cells to 96-well flat-bottom plates at 100 μl/well or 0.8−1.0×10⁴ cells/well; incubate ˜24 h at 37° C., 5% CO₂.

DAY 1

1. Make up two-fold drug titrations in separate 96-well plates, generally 50 μM on down to 0 μM. Use the same assay medium as mentioned in day 0, step 2, above. Titrations are made by adding 90 μl/well of drug at 200 μM (4× the final well concentration) to the top well of a particular plate column. Since the stock drug concentration is usually 20 mM in DMSO, the 200 μM drug concentration contains 2% DMSO.

Therefore, diluent made up to 2% DMSO in assay medium (F12K+0.5% fetal bovine serum) is used as diluent for the drug titrations in order to dilute the drug but keep the DMSO concentration constant. Add this diluent to the remaining wells in the column at 60 μl/well. Take 60 μl from the 120 μl of 200 μM drug dilution in the top well of the column and mix with the 60 μl in the second well of the column. Take 60 μl from this well and mix with the 60 μl in the third well of the column, and so on until two-fold titrations are completed. When the next-to-the-last well is mixed, take 60 μl of the 120 μl in this well and discard it. Leave the last well with 60 μl of DMSO/media diluent as a non-drug-containing control. Make 9 columns of titrated drug, enough for triplicate wells each for 1) VEGF (obtained from Pepro Tech Inc., catalogue no. 100-200, 2) endothelial cell growth factor (ECGF) (also known as acidic fibroblast growth factor, or aFGF) (obtained from Boehringer Mannheim Biochemica, catalogue no. 1439 600); or, 3) human PDGF B/B (1276-956, Boehringer Mannheim, Germany) and assay media control. ECGF comes as a preparation with sodium heparin.

2. Transfer 50 μl/well of the drug dilutions to the 96-well assay plates containing the 0.8−1×10⁴ cells/100 μl/well of the HUV-EC-C cells from day 0 and incubate ˜2 h at 37° C., 5% CO₂.

3. In triplicate, add 50 μl/well of 80 μg/ml VEGF, 20 ng/ml ECGF, or media control to each drug condition. As with the drugs, the growth factor concentrations are 4× the desired final concentration. Use the assay media from day 0, step 2, to make the concentrations of growth factors. Incubate approximately 24 hours at 37° C., 5% CO₂. Each well will have 50 μl drug dilution, 50 μl growth factor or media, and 100 μl cells, =200 μl /well total. Thus the 4× concentrations of drugs and growth factors become 1× once everything has been added to the wells.

DAY 2

1. Add ³H-thymidine (Amersham; catalogue no. TRK-686) at 1 μCi/well (10 μl/well of 100 μCi/ml solution made up in RPMI media+10% heat-inactivated fetal bovine serum) and incubate ˜24 h at 37° C., 5% CO₂. Note: ³H-thymidine is made up in RPMI media because all of the other applications for which we use the ³H-thymidine involve experiments done in RPMI. The media difference at this step is probably not significant. RPMI was obtained from Gibco BRL, catalogue no.11875-051.

DAY 3

1. Freeze plates overnight at −20° C.

DAY 4

1. Thaw plates and harvest with a 96-well plate harvester (Tomtec Harvester 96®) onto filter mats (Wallac; catalogue no. 1205-401); read counts on a Wallac Betaplate™ liquid scintillation counter.

### CONCLUSION

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations that are not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for × being bromine and chlorine are fully described.

In view of the degeneracy of the genetic code, other combinations of nucleic acids also encode the claimed peptides and proteins of the invention. For example, all four nucleic acid sequences GCT, GCC, GCA, and GCG encode the amino acid alanine. Therefore, if for an amino acid there exists an average of three codons, a polypeptide of 100 amino acids in length will, on average, be encoded by 3100, or 5×1047, nucleic acid sequences. Thus, a nucleic acid sequence can be modified to form a second nucleic acid sequence, encoding the same polypeptide as encoded by the first nucleic acid sequences, using routine procedures and without undue experimentation. Thus, all possible nucleic acids that encode the claimed peptides and proteins are also fully described herein, as if all were written out in full taking into account the codon usage, especially that preferred in humans. Furthermore, changes in the amino acid sequences of polypeptides, or in the corresponding nucleic acid sequence encoding such polypeptide, may be designed or selected to take place in an area of the sequence where the significant activity of the polypeptide remains unchanged. For example, an amino acid change may take place within a β-turn, away from the active site of the polypeptide. Also changes such as deletions (e.g. removal of a segment of the polypeptide, or in the corresponding nucleic acid sequence encoding such polypeptide, which does not affect the active site) and additions (e.g. addition of more amino acids to the polypeptide sequence without affecting the function of the active site, such as the formation of GST-fusion proteins, or additions in the corresponding nucleic acid sequence encoding such polypeptide without affecting the function of the active site) are also within the scope of the present invention. Such changes to the polypeptides can be performed by those with ordinary skill in the art using routine procedures and without undue experimentation. Thus, all possible nucleic and/or amino acid sequences that can readily be determined not to affect a significant activity of the peptide or protein of the invention are also fully described herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims.

1.-9. (canceled)

10. An antibody or antibody fragment having specific binding affinity to a kinase polypeptide or to a domain of said polypeptide, wherein said polypeptide is a kinase polypeptide having an amino acid sequence selected from the group of those set forth in SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO:68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110,SEQ ID NO: l1, SEQ ID NO: 112, SEQ ID NO: 113, and SEQ ID NO: 114.

11. A hybridoma which produces an antibody having specific binding affinity to a kinase polypeptide having an amino acid sequence selected from the group consisting of those set forth in SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO:68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, and SEQ ID NO: 114.

12. A kit comprising an antibody which binds to an isolated, enriched or purified kinase polypeptide that comprises an amino acid sequence having
(a) an amino acid sequence selected from the group consisting of those set forth in SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO:68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, and SEQ ID NO: 114, respectively; or
(b) an amino acid sequence selected from the group consisting of those set forth in SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO:68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, and SEQ ID NO: 114, respectively, except that it lacks one or more, but not all of the domains selected from the group consisting of an N-terminal domain, a C-terminal catalytic domain, a catalytic domain, a C-terminal domain, a coiled-coil structure region, a proline-rich region, a spacer region, and a C-terminal tail,
and negative control antibody.

13.-28. (canceled)

29. The kit of claim 12, wherein the polypeptide is isolated, purified or enriched from a mammal.
